(19) United States
(12) Patent Application Publication
(10) Pub. No.: US 2026/0250302 A1
(43) Pub. Date: Aug. 27, 2026

(54) ANTI-VIRAL COMPOUNDS

(71) Applicants: Aligos Therapeutics, Inc., South San Francisco, CA (US); Katholieke Universiteit Leuven, Leuven (BE)

(72) Inventors: David McGowan, Brussels (BE); David Bernard Smith, San Mateo, CA (US); Dorothée Alice Marie-Eve Bardiot, Leuven (BE); Arnaud Didier Marie Marchand, Leuven (BE)

(21) Appl. No.: 19/538,827

(22) Filed: Feb. 12, 2026

Related U.S. Application Data
(60) Provisional application No. 63/758,579, filed on Feb. 14, 2025.

(51) Int. Cl. C07D 519/00; C07D 403/12; C07D 405/14

(52) U.S. Cl. CPC C07D 519/00; C07D 403/12; C07D 405/14

(57) ABSTRACT

Provided herein are compounds of Formula (I), or pharmaceutically acceptable salts thereof, pharmaceutical compositions that include a compound described herein (including pharmaceutically acceptable salts of a compound described herein) and methods of synthesizing the same. Also provided herein are methods of treating diseases and/or conditions with a compound of Formula (I), or a pharmaceutically acceptable salt thereof.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/758,579, filed Feb. 14, 2025, which is hereby expressly incorporated herein by reference in its entirety.

### BACKGROUND

### Field

The present application relates to the fields of chemistry, biochemistry and medicine. Disclosed herein are compounds of Formula (I), or pharmaceutically acceptable salt thereof, pharmaceutical compositions that include a compound described herein (including pharmaceutically acceptable salts of a compound described herein) and methods of synthesizing the same. Also disclosed herein are methods of treating diseases and/or conditions with a compound of Formula (I), or a pharmaceutically acceptable salt thereof.

### Description

A positive-sense single-stranded RNA virus ((+)ssRNA virus) is a virus that uses positive sense, single stranded, RNA as its genetic material. Positive-sense single-stranded RNA viruses can be enveloped or non-enveloped. Coronaviridae, Picornaviridae and Norviruses are each a (+)ssRNA virus. Each of the aforementioned viruses are known to infect mammals, including humans.

### SUMMARY

Some embodiments disclosed herein relate to a compound of Formula (I), or a pharmaceutically acceptable salt thereof.

Some embodiments disclosed herein relate to a pharmaceutical composition that can contain an effective amount of a compound of Formula (I), or a pharmaceutically acceptable salt thereof.

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a coronavirus.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a picornavirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a norovirus.

These are other embodiments are described in greater detail below.

### DETAILED DESCRIPTION

Coronaviridae viruses are a family of enveloped, positive-stranded, single-stranded, spherical RNA viruses. Coronaviruses are named for the crown-like spikes on their surface. The Coronaviridae family includes two sub-families, Coronavirus and Torovirus. The Coronavirus genus has a helical nucleocapsid, and Torovirus genus has a tubular nucleocapsid. The Coronaviridae family of viruses includes Middle East respiratory syndrome coronavirus (MERS-CoV), SARS and SARS-CoV-2.

Coronavirus disease 2019 (COVID-19) (also referred to as novel coronavirus pneumonia or 2019-nCoV acute respiratory disease) is an infectious disease caused by the virus severe acute respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. There is not yet a vaccine or specific antiviral treatment for COVID-19 and accordingly, there is a pressing need for treatments or cures.

SARS-CoV-2 is not the only coronavirus that causes disease. It is a 3-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. β-coronavirus can also manifest as zoonotic infections, spread to and from humans and animals. Additionally, non-human species such as camels, bats, tigers, non-human primates, and rabbits can be susceptible to β-coronavirus. Accordingly, there is a pressing need for treatments or cures to multiple coronaviruses.

The present disclosure provides molecules useful against coronaviruses, and especially SARS-CoV-2, the causative agent of COVID-19 in humans. Accordingly, the present disclosure fulfills the need in the art for compounds that can be safely and effectively treat or prevent coronavirus infections in humans.

Picornaviruses are a family of positive strand RNA, nonenveloped viruses. A picornavirus has 60 identical subunits (vertices) which contain five protomers. Each protomer is made up of one copy of four proteins, named VP1, VP2, VP3 and VP4. There are several genera of picornaviruses, including, Enterovirus, Aphthovirus, Cardiovirus and Hepatovirus. Enteroviruses known to infect human includes, but are not limited to, Rhinovirus A, Rhinovirus B, Rhinovirus C, Coxsackievirus A, Coxsackievirus B and Poliovirus. There is no specific treatment for a picornavirus infection.

Noroviruses are single-stranded positive-sense RNA, non-enveloped viruses belonging to the Caliciviridae family. Noroviruses are often spread by the fecal-oral route and are a common cause of gastroenteritis. Infected subjects can experience nausea, non-bloody diarrhea, vomiting and/or abdominal pain. Those suffering from a norovirus infection can become severely dehydrated and require medical attention. As with a picornavirus infection, there is no specific treatment for a norovirus infection. Accordingly, there is a need for compounds that effectively treat or prevent a picornavirus and/or a norovirus infection.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3) individually and independently selected from deuterium, alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, C-amido(alkyl), isocyanato, thiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amine and a di-substituted amine.

As used herein, “Cₐ to C_{b}”, “Cₐ-C_{b}” or “Cₐ-b” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused- or spiro-fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused- or spiro-fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused- or spiro-fashion. Additionally, any nitrogens in a heterocyclyl may be quaternized. Heterocyclyl groups may be unsubstituted or substituted. Examples of such “heterocyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidinone, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., tetrahydroquinoline and 3,4-methylenedioxyphenyl).

As used herein, “cycloalkyl(alkyl)” refers to a cycloalkyl group connected, as a substituent, via a lower alkylene group. The lower alkylene and cycloalkyl group of a cycloalkyl(alkyl) may be substituted or unsubstituted. A cycloalkyl(alkyl) group may be unsubstituted or substituted.

As used herein, “aryl(alkyl)” refers to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

A “heterocyclyl(alkyl)” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted.” Further, when a lower alkylene group is substituted, the lower alkylene can be substituted by replacing both hydrogens on the same carbon with a cycloalkyl group

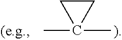

As used herein, “alkoxy” refers to the formula —OR wherein R is an alkyl, an alkenyl, an alkynyl, a cycloalkyl, a cycloalkenyl, an aryl, a heteroaryl, a heterocyclyl, a cycloalkyl(alkyl), an aryl(alkyl), a heteroaryl(alkyl) or a heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzyloxy. In some instances, an alkoxy can be —OR, wherein R is an unsubstituted C₁₋₄ alkyl. An alkoxy may be substituted or unsubstituted.

As used herein, “acyl” refers to a hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl) connected, as substituents, via a carbonyl group. Examples include formyl, acetyl, propanoyl, benzoyl and acryl. An acyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl, 2-fluoroisobutyl and —CF₂CF₃. A haloalkyl may be substituted or unsubstituted.

As used herein, “haloalkoxy” refers to a O-alkyl group and O-monocyclic cycloalkyl group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy, 2-fluoroisobutoxy, chloro-substituted cyclopropoxy, fluoro-substituted cyclopropoxy, chloro-substituted cyclobutoxy and fluoro-substituted cyclobutoxy. In some instances, a haloalkoxy can be —OR, wherein R is a C₁₋₄ alkyl substituted by 1, 2 or 3 halogens. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “—S(═O)₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CS(═O)₂—” group wherein each X is a halogen.

A “trihalomethanesulfonamido” group refers to an “X₃CS(═O)₂N(R_{A})—” group wherein each X is a halogen, and R_{A} is hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—SCN” group.

An “isothiocyanato” group refers to an “—NCS” group.

A “mercapto” group refers to an “—SH” group.

A “carbonyl” group refers to a —C(═O)— group.

An “S-sulfonamido” group refers to a “—S(═O)₂N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An S-sulfonamido may be substituted or unsubstituted.

An “N-sulfonamido” group refers to a “RS(═O)₂N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-carbamyl may be substituted or unsubstituted.

An “O-thiocarbamyl” group refers to a “—OC(═S)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-amido may be substituted or unsubstituted.

A “mono-substituted amine” refers to a “—NHR_{A}” in which R_{A} can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NHR_{A}, wherein R_{A} can be an unsubstituted C₁₋₆ alkyl or an unsubstituted or a substituted benzyl.

A “di-substituted amine” refers to a “—NR_{A}R_{B}” in which R_{A} and R_{B} can be independently can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NR_{A}R_{B}, wherein R_{A} and R_{B} can be independently an unsubstituted C₁₋₆ alkyl or an unsubstituted or a substituted benzyl.

A “ketoamide” group refers to a —C(═O)—C(═O)N(R_{A}R_{B}) group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A ketoamide may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, the term “fused” refers to two rings which have two atoms and one bond in common. As used herein, the term “spiro” refers to two rings which have one atom in common. As used herein, the term “bridged” refers to two rings which contains a linkage of one or more atoms connecting non-adjacent atoms.

Where the numbers of substituents are not specified (e.g., haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

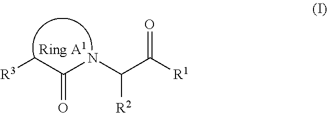

wherein: R¹ can be selected from

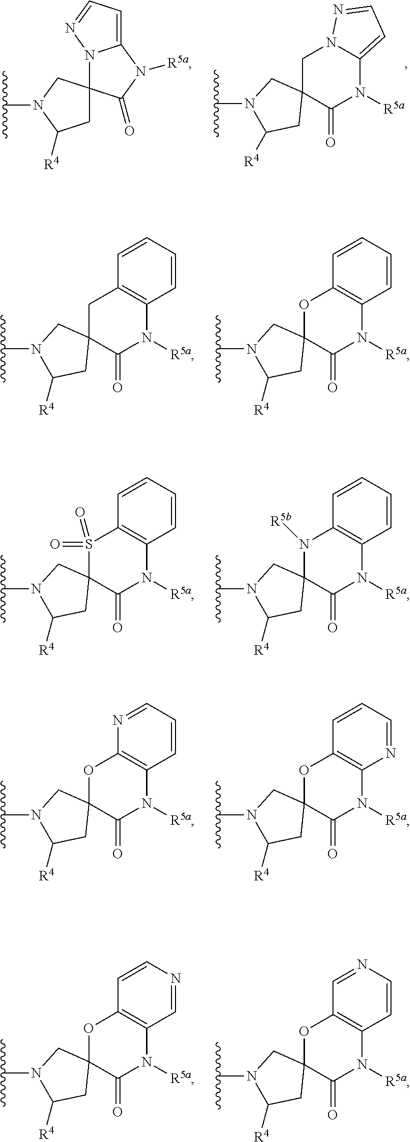

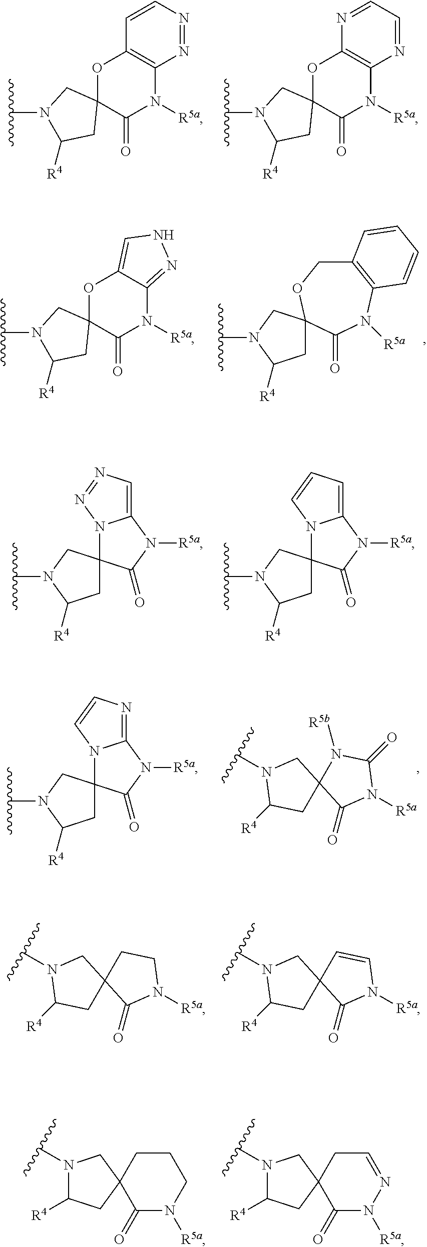

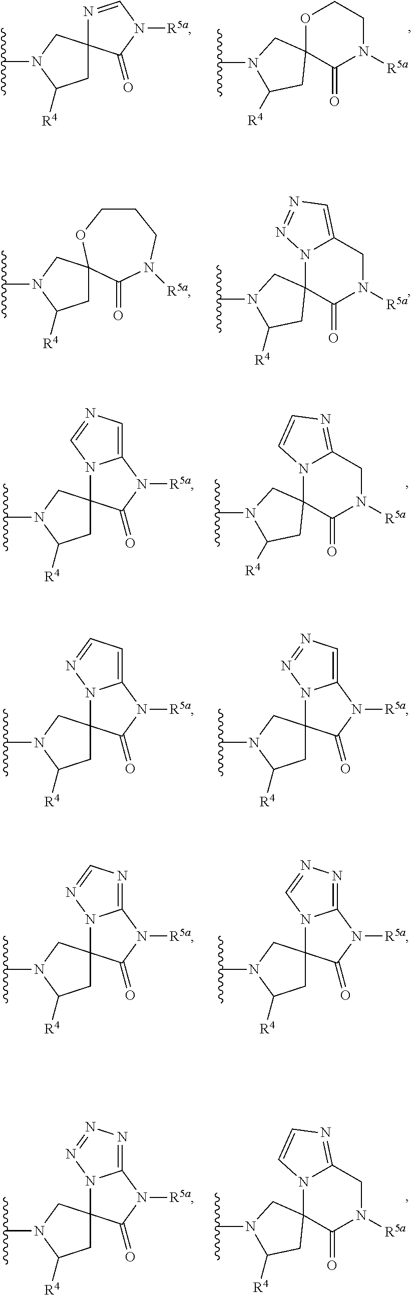

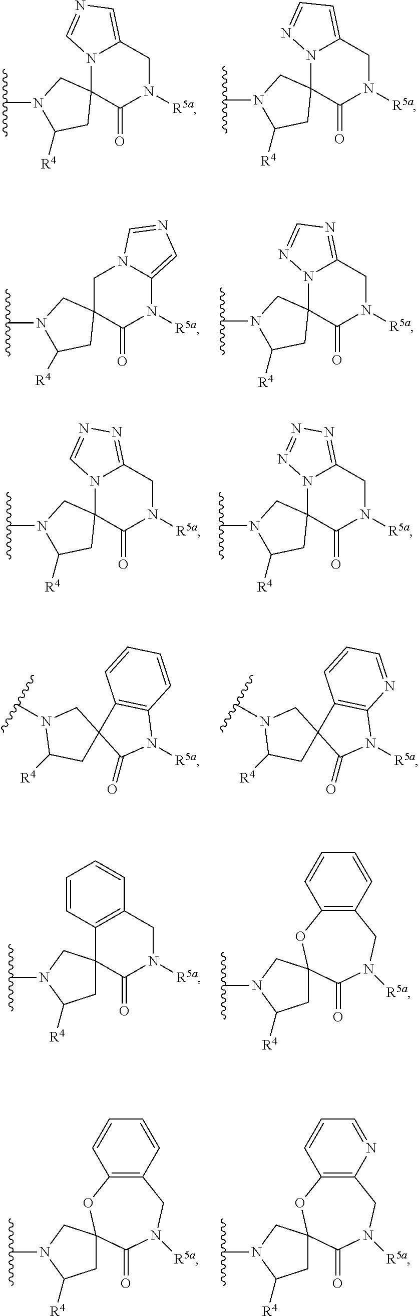

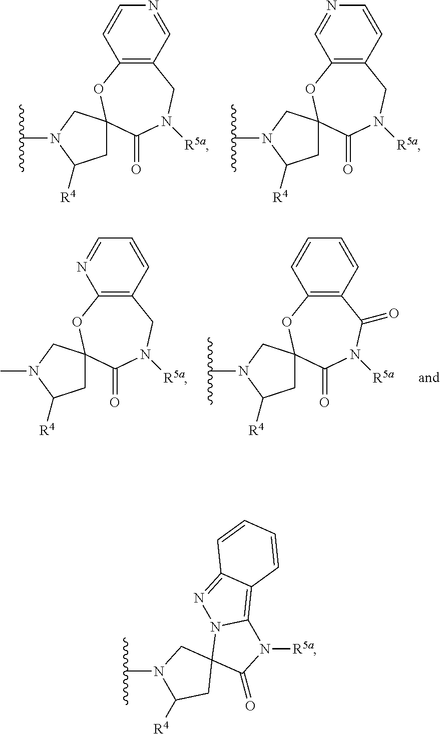

wherein each can be optionally substituted with one or more moieties independently selected from halogen, cyano, hydroxy, an unsubstituted C₁₋₆ alkyl, an unsubstituted —O(C₁₋₆ alkyl), an unsubstituted C₁₋₄ haloalkyl, an unsubstituted C₁₋₄ haloalkoxy, an unsubstituted or a substituted phenoxy, an unsubstituted or a substituted C₃₋₆ cycloalkyl, an unsubstituted or a substituted phenyl, an unsubstituted or a substituted benzyl, an unsubstituted or a substituted 5- or 6-membered heteroaryl, —S(═O)₂ (an unsubstituted C₁₋₄ alkyl), —NR^{N1}R^{N2} and —C(═O)—NR^{N1}R^{N2}, wherein R^{N1} and R^{N2} can be independently hydrogen or an unsubstituted C₁₋₄ alkyl, or R^{N1} and R^{N2} can be taken together to form a monocyclic heterocyclyl; R² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₁₀ cycloalkyl, an unsubstituted or a substituted C₃₋₁₀ cycloalkenyl, an unsubstituted or a substituted aryl, an unsubstituted or a substituted aryl(alkyl), an unsubstituted or a substituted heteroaryl, an unsubstituted or a substituted heteroaryl(alkyl), an unsubstituted or a substituted heterocyclyl or an unsubstituted or a substituted heterocyclyl(alkyl); R³ can be

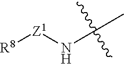

or R⁹; Ring A¹ can be a 5-, 6-, 7-, 8- or 9-membered heterocyclyl that optionally includes a double bond and optionally fused to an unsubstituted or a substituted monocyclic C₃₋₄ cycloalkyl; Z¹ can be —C(═O)— or —S(═O)₂—; R⁴ can be selected from cyano, an unsubstituted or a substituted C₂₋₅ alkynyl, an unsubstituted or a substituted acyl, an unsubstituted or a substituted ketoamide, —C(═O)NH₂, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂); R^{5a} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; R^{5b} can be selected from hydrogen, an unsubstituted or a substituted C₁₋₄ alkyl, an unsubstituted or a substituted C₂₋₄ alkenyl and an unsubstituted or a substituted C₃₋₆ cycloalkyl; each R⁶ and each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl); R⁸ can be selected from an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic heteroaryl, an unsubstituted or a substituted monocyclic heterocyclyl, an unsubstituted or a substituted alkoxy and —NR¹⁴R¹⁵, wherein the substituted C₁₋₆ alkyl can be substituted 1 or 2 times with a substituent selected from hydroxy and an unsubstituted C₁₋₄ alkoxy, wherein the substituted monocyclic C₃₋₆ cycloalkyl can be substituted 1, 2, 3 or 4 times with a substituent independently selected from halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl is substituted 1 or 2 times with a substituent independently selected from an unsubstituted C₁₋₄ alkoxy and an unsubstituted or a substituted C₃₋₆ cycloalkyl; R⁹ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 12-membered monocyclic heterocyclyl, an unsubstituted or a substituted 5- to 12-membered bicyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹⁰, —NR¹¹R¹² or —C(═O)—NR^{13A}R^{13B}; R¹⁰ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); R¹¹ are R¹² can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); R^{13A} can be hydrogen or an unsubstituted C₁₋₃ alkyl; R^{13B} can be an unsubstituted or a substituted aryl, an unsubstituted or a substituted heteroaryl or an unsubstituted or a substituted 3- to 8-membered monocyclic heterocyclyl; R¹⁴ and R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or R¹⁴ and R¹⁵ are taken together along with the nitrogen to which they are connected to form an unsubstituted or a substituted 3-8 membered heterocyclyl.

Various rings indicated with Ring A¹ can be present in a compound of Formula (I), or a pharmaceutically acceptable salt thereof. As provided herein, Ring A can include one or more double bonds (for example 1 or 2 double bonds) and/or be fused to an unsubstituted cyclopropyl, an unsubstituted cyclobutyl, a substituted cyclopropyl or a substituted cyclobutyl. Ring A and/or the monocyclic C₃₋₄ cycloalkyl that can be fused to Ring A can be further substituted. For example, Ring A and/or the monocyclic C₃₋₄ cycloalkyl that can be fused to Ring A can be substituted 1, 2, 3 or 4 times with a moiety selected from methyl, ethyl, hydroxy, fluoro, chloro, bromo, iodo, methoxy and ethoxy.

In some embodiments, Ring A¹ can be a 5-membered heterocyclyl that does not include a double bond and is not fused to an unsubstituted or a substituted monocyclic C₃₋₄ cycloalkyl. In other embodiments, Ring A¹ can be a 5-membered heterocyclyl that does include a double bond and is not fused to an unsubstituted or a substituted monocyclic C₃₋₄ cycloalkyl. In still other embodiments, Ring A¹ can be a 5-membered heterocyclyl that does not include a double bond and is fused to an unsubstituted or a substituted monocyclic C₃₋₄ cycloalkyl. In yet still other embodiments, Ring A¹ can be a 5-membered heterocyclyl that does include a double bond and is fused to an unsubstituted or a substituted monocyclic C₃₋₄ cycloalkyl. In some embodiments, Ring A¹ can be a 6-membered heterocyclyl that does not include a double bond and is not fused to an unsubstituted or a substituted monocyclic C₃₋₄ cycloalkyl. In other embodiments, Ring A¹ can be a 6-membered heterocyclyl that does include a double bond and is not fused to an unsubstituted or a substituted monocyclic C₃₋₄ cycloalkyl. In still other embodiments, Ring A¹ can be a 6-membered heterocyclyl that does not include a double bond and is fused to an unsubstituted or a substituted monocyclic C₃₋₄ cycloalkyl. In yet still other embodiments, Ring A¹ can be a 6-membered heterocyclyl that does include a double bond and is fused to an unsubstituted or a substituted monocyclic C₃₋₄ cycloalkyl. In some embodiments, Ring A¹ can be a 7-membered heterocyclyl that does not include a double bond and is not fused to an unsubstituted or a substituted monocyclic C₃₋₄ cycloalkyl. In other embodiments, Ring A¹ can be a 7-membered heterocyclyl that does include a double bond and is not fused to an unsubstituted or a substituted monocyclic C₃₋₄ cycloalkyl. In still other embodiments, Ring A¹ can be a 7-membered heterocyclyl that does not include a double bond and is fused to an unsubstituted or a substituted monocyclic C₃₋₄ cycloalkyl. In yet still other embodiments, Ring A¹ can be a 7-membered heterocyclyl that does include a double bond and is fused to an unsubstituted or a substituted monocyclic C₃₋₄ cycloalkyl. In some embodiments, Ring A¹ can be an 8-membered heterocyclyl that does not include a double bond and is not fused to an unsubstituted or a substituted monocyclic C₃₋₄ cycloalkyl. In other embodiments, Ring A¹ can be an 8-membered heterocyclyl that does include a double bond and is not fused to an unsubstituted or a substituted monocyclic C₃₋₄ cycloalkyl. In still other embodiments, Ring A¹ can be an 8-membered heterocyclyl that does not include a double bond and is fused to an unsubstituted or a substituted monocyclic C₃₋₄ cycloalkyl. In yet still other embodiments, Ring A¹ can be an 8-membered heterocyclyl that does include a double bond and is fused to an unsubstituted or a substituted monocyclic C₃₋₄ cycloalkyl. In some embodiments, Ring A¹ can be a 9-membered heterocyclyl that does not include a double bond and is not fused to an unsubstituted or a substituted monocyclic C₃₋₄ cycloalkyl. In other embodiments, Ring A¹ can be a 9-membered heterocyclyl that includes a double bond and is not fused to an unsubstituted or a substituted monocyclic C₃₋₄ cycloalkyl. In still other embodiments, Ring A¹ can be a 9-membered heterocyclyl that does not include a double bond and is fused to an unsubstituted or a substituted monocyclic C₃₋₄ cycloalkyl. In yet still other embodiments, Ring A¹ can be a 9-membered heterocyclyl that does include a double bond and is fused to an unsubstituted or a substituted monocyclic C₃₋₄ cycloalkyl. Examples of Ring A¹ include, but are not limited to, the following:

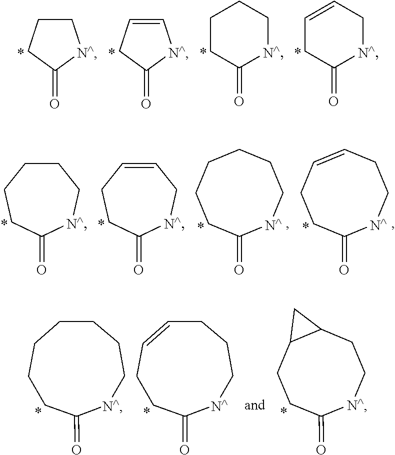

wherein the carbon indicated with an asterisk (“*”) is the carbon connected to R³ and the nitrogen indicated with “A” is the nitrogen shown in Formula (I).

Various moieties can be present on Ring A¹. A non-limiting list of moieties that can be present on Ring A¹ include halogen (e.g., F or Cl), methyl, hydroxy, methoxy and —CF₃. The number of moieties can vary. In some embodiments, the number of moieties that can be present on Ring A¹ can be 1, 2, 3, 4, 5 or 6. When more than one moieties is present, the moieties can be the same or different.

As provided herein, in some embodiments, R¹ can be selected from

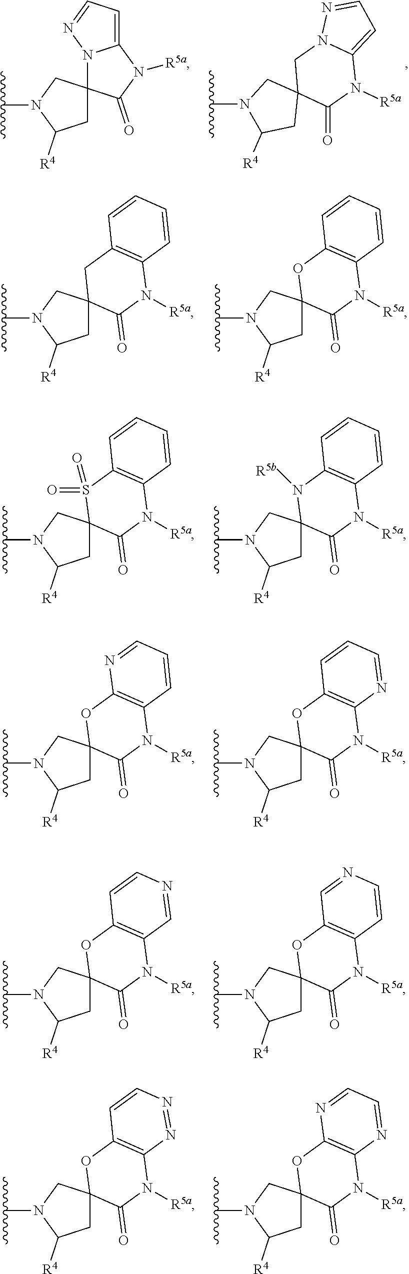

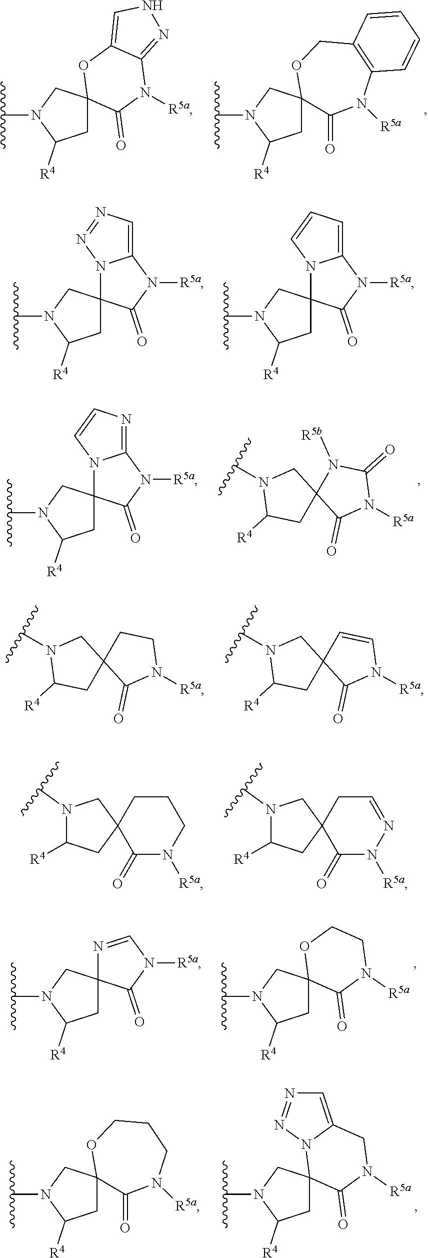

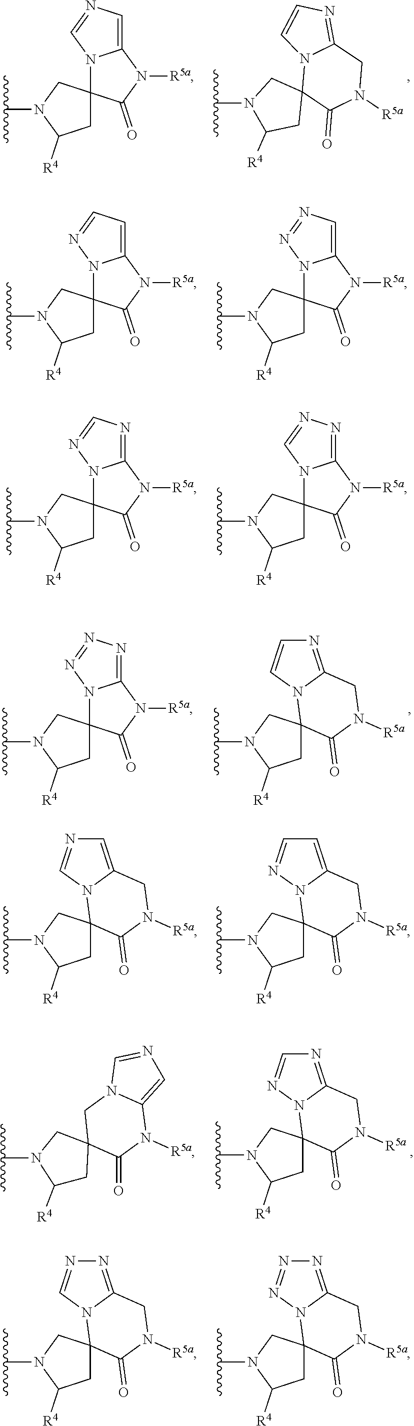

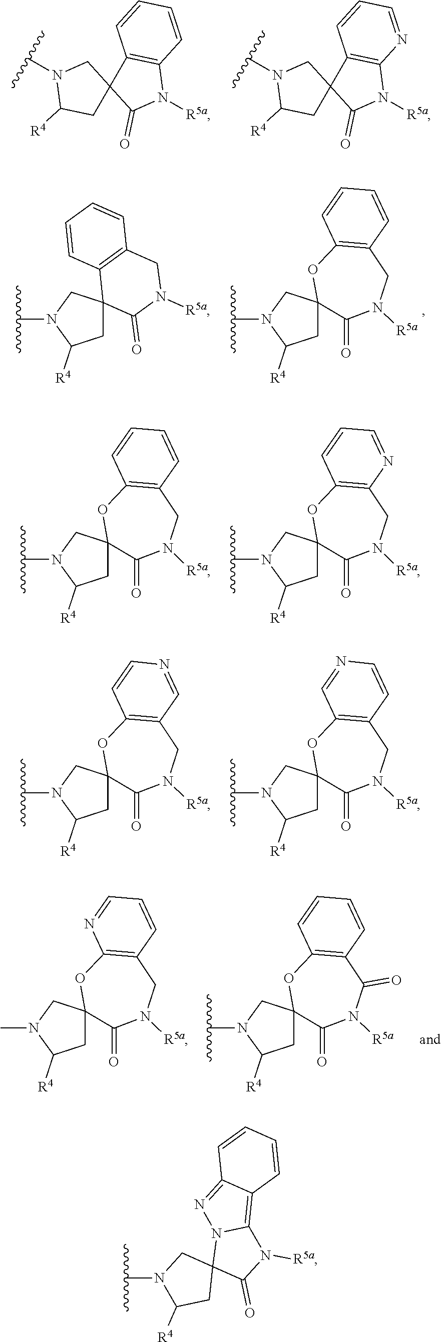

wherein each is optionally substituted with one or more moieties independently selected from the group consisting of halogen, cyano, hydroxy, an unsubstituted C₁₋₆ alkyl, an unsubstituted —O(C₁₋₆ alkyl), an unsubstituted C₁₋₄ haloalkyl, an unsubstituted C₁₋₄ haloalkoxy, an unsubstituted or a substituted phenoxy, an unsubstituted or a substituted C₃₋₆ cycloalkyl, an unsubstituted or a substituted phenyl, an unsubstituted or a substituted benzyl, an unsubstituted or a substituted 5- or 6-membered heteroaryl, —S(═O)₂ (an unsubstituted C₁₋₄ alkyl), —NR^{N1}R^{N2} and —C(═O)—NR^{N1}R^{N2}, wherein R^{N1} and R^{N2} are independently hydrogen or an unsubstituted C₁₋₄ alkyl or R^{N1} and R^{N2} are taken together to form a monocyclic heterocyclyl.

In some embodiments, R^{5a} can be hydrogen. As provided herein, R^{5a} can be a non-hydrogen moiety. For example, R^{5a} can be an unsubstituted or a substituted C₁₋₄ alkyl, an unsubstituted or a substituted C₂₋₄ alkenyl and an unsubstituted or a substituted C₃₋₆ cycloalkyl. In some embodiments, R^{5a} can be an unsubstituted C₁₋₄ alkyl. In other embodiments, R^{5a} can be a substituted C₁₋₄ alkyl. In still other embodiments, R^{5a} can be an unsubstituted C₂₋₄ alkenyl. In yet still other embodiments, R^{5a} can be a substituted C₂₋₄ alkenyl. In some embodiments, R^{5a} can be an unsubstituted C₃₋₆ cycloalkyl. In other embodiments, R^{5a} can be a substituted C₃₋₆ cycloalkyl. For example, R^{5a} can be an unsubstituted or a substituted monocyclic C₃₋₆ cycloalkyl. In some embodiments, R^{5a} can be methyl. In some embodiments, R^{5a} can be cyclopropyl.

As provided herein, R¹ can be optionally substituted with one or more moieties (such as 1, 2 or 3 moieties) selected from halogen, cyano, hydroxy, an unsubstituted C₁₋₆ alkyl, an unsubstituted —O(C₁₋₆ alkyl), an unsubstituted C₁₋₄ haloalkyl, an unsubstituted C₁₋₄ haloalkoxy, an unsubstituted or a substituted phenoxy, an unsubstituted or a substituted C₃₋₆ cycloalkyl, an unsubstituted or a substituted phenyl, an unsubstituted or a substituted benzyl, an unsubstituted or a substituted 5- or 6-membered heteroaryl, —S(═O)₂ (an unsubstituted C₁₋₄ alkyl), —NR^{N1}R^{N2} and —C(═O)—NR^{N1}R^{N2}, wherein R^{N1} and R^{N2} are independently hydrogen or an unsubstituted C₁₋₄ alkyl or R^{N1} and R^{N2} are taken together to form a monocyclic heterocyclyl. Exemplary groups that can be present on R¹ can be selected from bromo, chloro, fluoro, cyano, hydroxy, methyl, ethyl, n-propyl, iso-propyl, n-butyl, iso-butyl, sec-butyl, tert-butyl, pentyl (straight-chained and/or branched), hexyl (straight-chained and/or branched), methoxy, ethoxy, n-propoxy, iso-propoxy, n-butoxy, iso-butoxy, sec-butoxy, tert-butoxy, pentoxy (straight-chained and/or branched), hexoxy (straight-chained and/or branched), —CF₃, —CHF₂, —C(CH₃)F₂, —CH₂F, —CH(CH₃)F, —CH₂CF₃, —CH₂CH₂F, —CH₂CH₂CH₂F, —O(an unsubstituted C₁₋₄ haloalkyl) (such as —OCF₃, —OCHF₂, —OC(CH₃)F₂, —OCH₂F, —OCH(CH₃)F, —OCH₂CF₃, —OCH₂CH₂F and —OCH₂CH₂CH₂F), an unsubstituted phenoxy, a substituted phenoxy, an unsubstituted phenyl, a substituted phenyl, an unsubstituted benzyl, a substituted benzyl, an unsubstituted 5- or 6-membered monocyclic heteroaryl, a substituted 5- or 6-membered monocyclic heteroaryl, —S(═O)₂ (an unsubstituted C₁₋₁₄ alkyl), —NR^{N1}R^{N2} and —C(═O)—NR^{N1}R^{N2}, wherein R^{N1} and R^{N2} are independently hydrogen or an unsubstituted C₁₋₄ alkyl or R^{N1} and R^{N2} are taken together to form a monocyclic heterocyclyl (such as a 5- or 6-membered heterocyclyl that includes, but is not limited to, pyrrolidinyl, piperidinyl and morpholinyl).

In some embodiments, the unsubstituted or a substituted C₃₋₆ cycloalkyl that can be present on R¹ can be an unsubstituted or a substituted monocyclic C₃₋₆ cycloalkyl, such as cyclopropyl, cyclobutyl, cyclopentyl and cyclohexyl. In some embodiments, the unsubstituted or a substituted C₃₋₆ cycloalkyl that can be present on R¹ can be an unsubstituted or a substituted bicyclic C₃₋₆ cycloalkyl. For example, when R¹ is substituted with an unsubstituted or a substituted bicyclic C₃₋₆ cycloalkyl, the unsubstituted or a substituted bicyclic C₃₋₆ cycloalkyl can be an unsubstituted or a substituted spiro[2.2]pentane, an unsubstituted or a substituted spiro[2.3]hexane, an unsubstituted or a substituted bicyclo[1.1.1]pentane or an unsubstituted or a substituted bicyclo[2.1.1]hexane. The monocyclic C₃₋₆ cycloalkyl and bicyclic C₃₋₆ cycloalkyl that can be substituted on R¹ can be connected via 1 ring carbon of the C₃₋₆ cycloalkyl (for example,

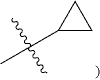 [SMILES: *C1CC1]

or a fused-fashion via 2 ring carbons of the C₃₋₆ cycloalkyl (for example,

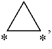

wherein the asterisks indicate the points of attachment) or spiro-fashion via 1 ring carbon of the C₃₋₆ cycloalkyl (for example,

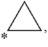

wherein the asterisk indicates the point of attachment). Examples of a C₃₋₆ cycloalkyl connected in a fused-fashion are

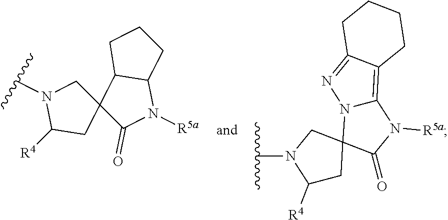

examples of a C₃₋₆ cycloalkyl spiro-connected to R¹ are

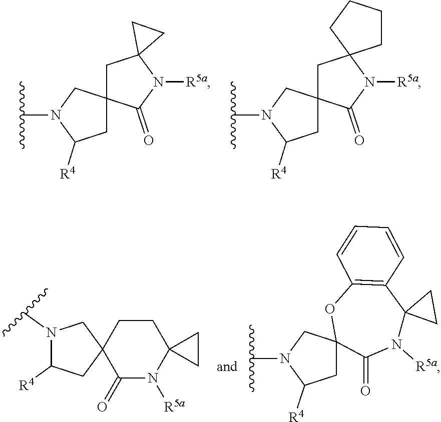

In some embodiments, R¹ can be substituted with an unsubstituted phenyl. In other embodiments, R¹ can be substituted with a substituted phenyl, such as a mono-substituted phenyl, a di-substituted phenyl or a phenyl substituted with 3 to 5 substituents. A non-limiting list of moieties that can be present on substituted phenyl that is substituted on R¹ include halogen (such as bromo, chloro and fluoro), cyano, an unsubstituted C₁₋₄ alkyl (for example, methyl, ethyl, n-propyl, iso-propyl, n-butyl, iso-butyl, sec-butyl and tert-butyl), hydroxy, an unsubstituted C₁₋₄ alkoxy (for example, —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, CH(CH₃)F, —CH₂CF₃, —CH₂CH₂F and —CH₂CH₂CH₂F), an unsubstituted C₁₋₄ haloalkoxy (for example, —O(C₁₋₄ haloalkyl) such as —OCF₃, —OCHF₂, —OC(CH₃)F₂, —OCH₂F, —OCH(CH₃)F, —OCH₂CF₃, —OCH₂CH₂F and —OCH₂CH₂CH₂F), an unsubstituted monocyclic C₃₋₆ cycloalkyl, a substituted monocyclic C₃₋₆ cycloalkyl, an unsubstituted phenyl, a substituted phenyl, an unsubstituted 5- or 6-membered heteroaryl and a substituted 5- or 6-membered heteroaryl (for example, a substituted monocyclic C₃₋₆ cycloalkyl, a substituted phenyl and/or a substituted 5- or 6-membered heteroaryl can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (for example —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂ (an unsubstituted C₁₋₄ alkyl.

Exemplary R¹ groups include the following:

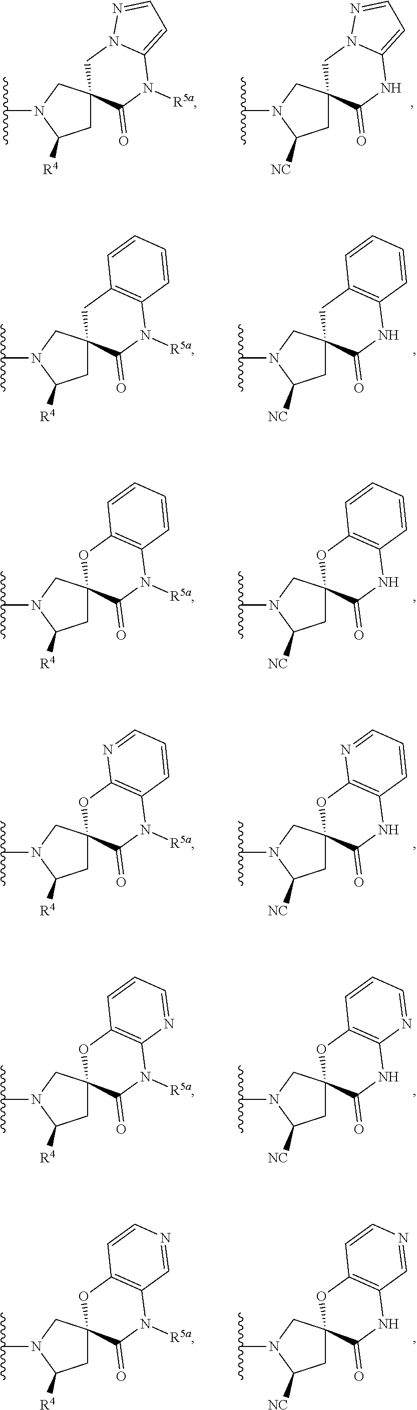

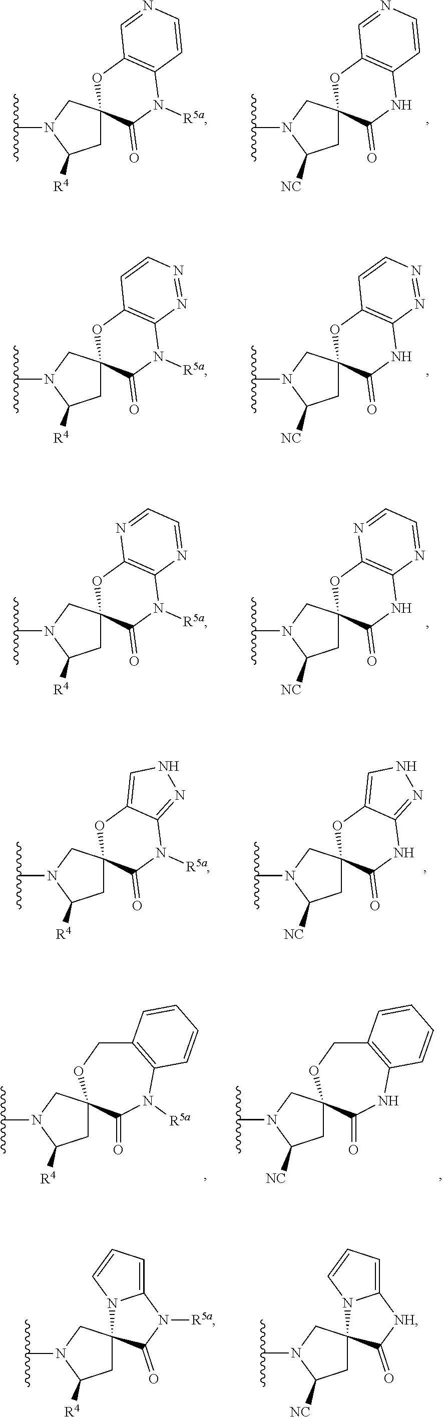

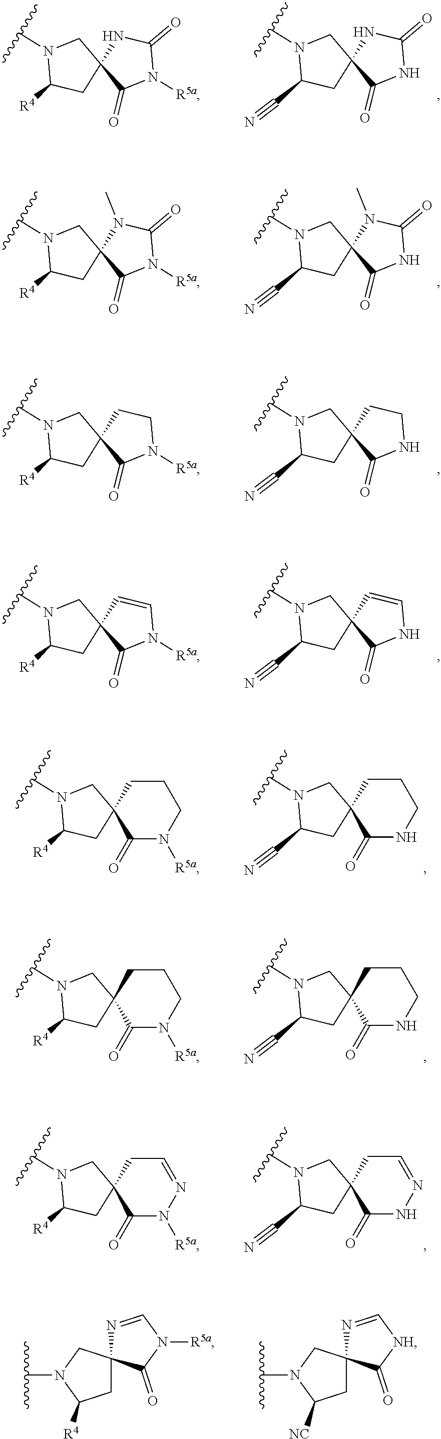

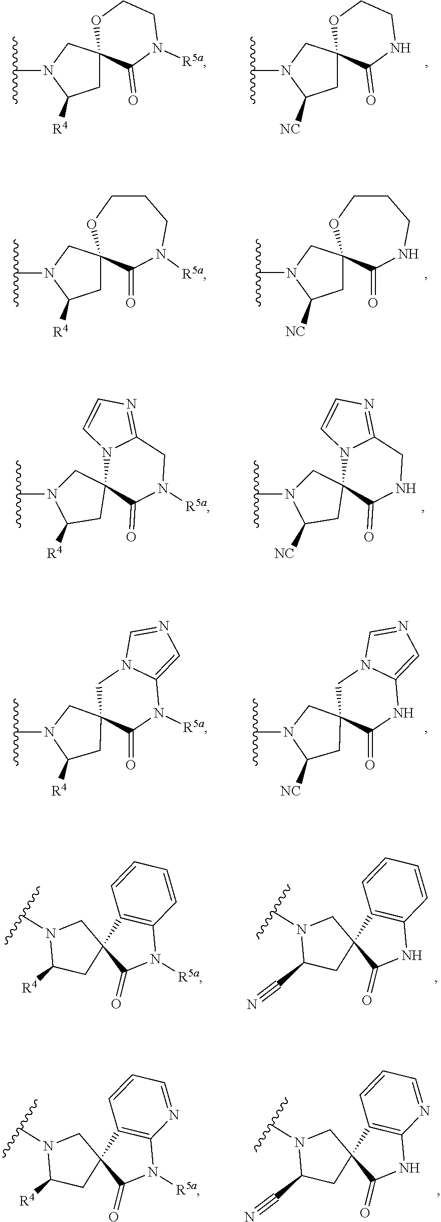

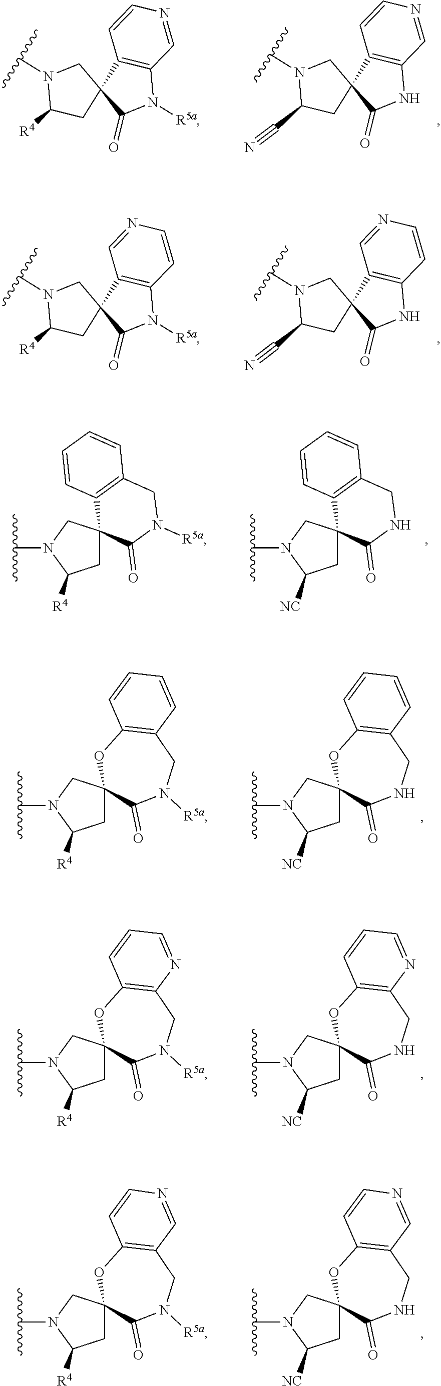

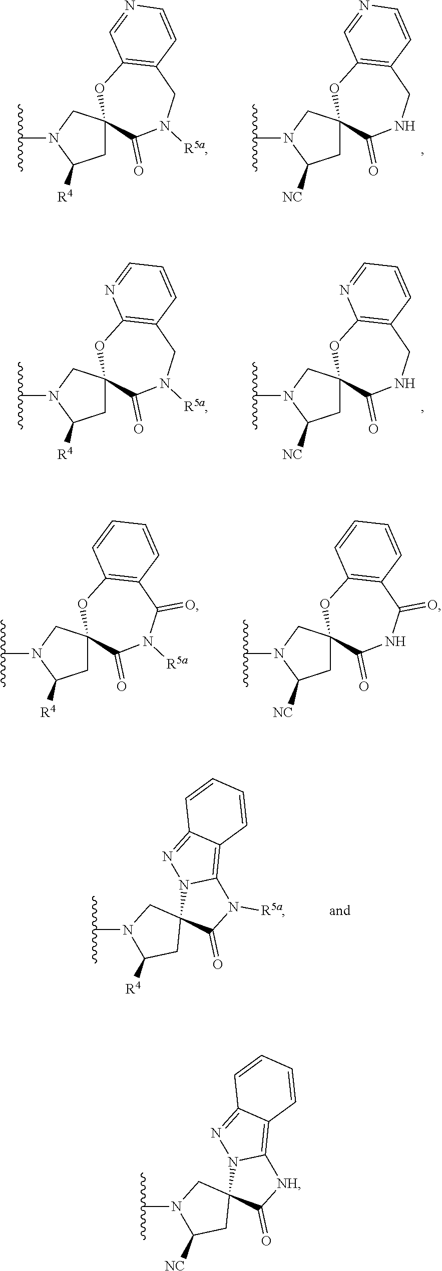

wherein each can be unsubstituted or substituted, including the replacement of the hydrogen on a nitrogen.

Examples of substituted version include the following:

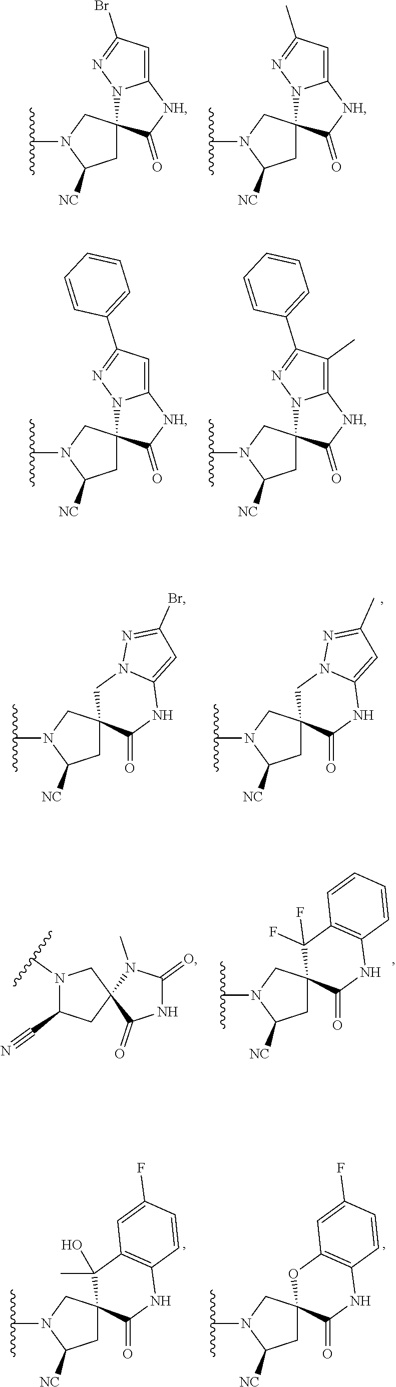

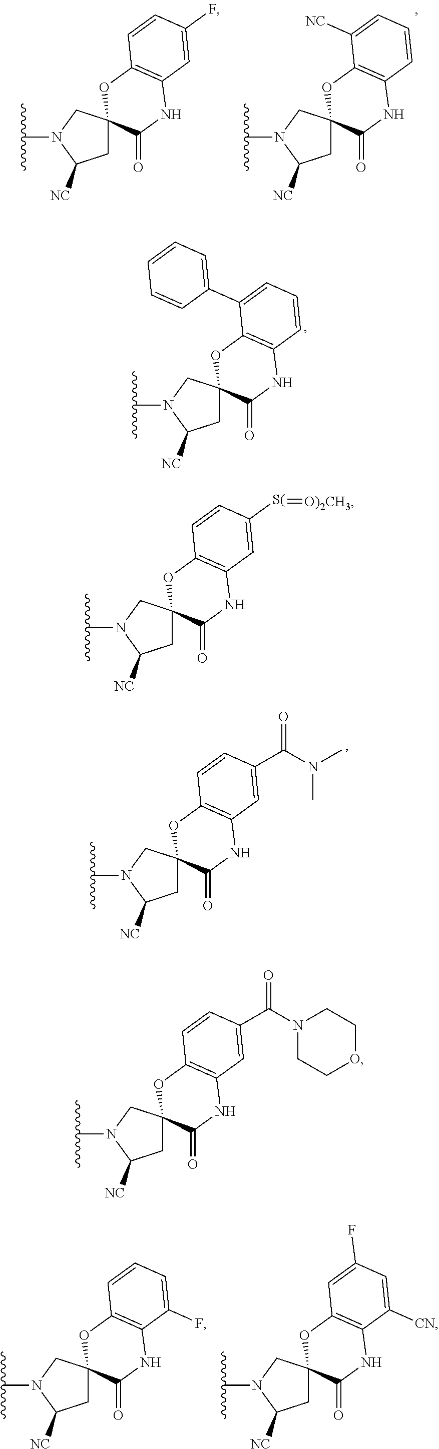

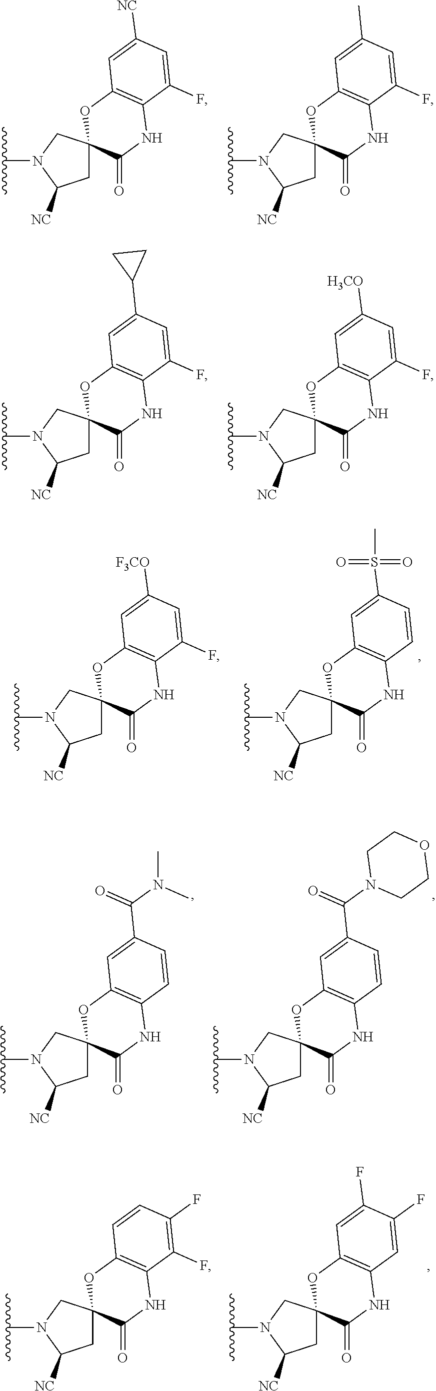

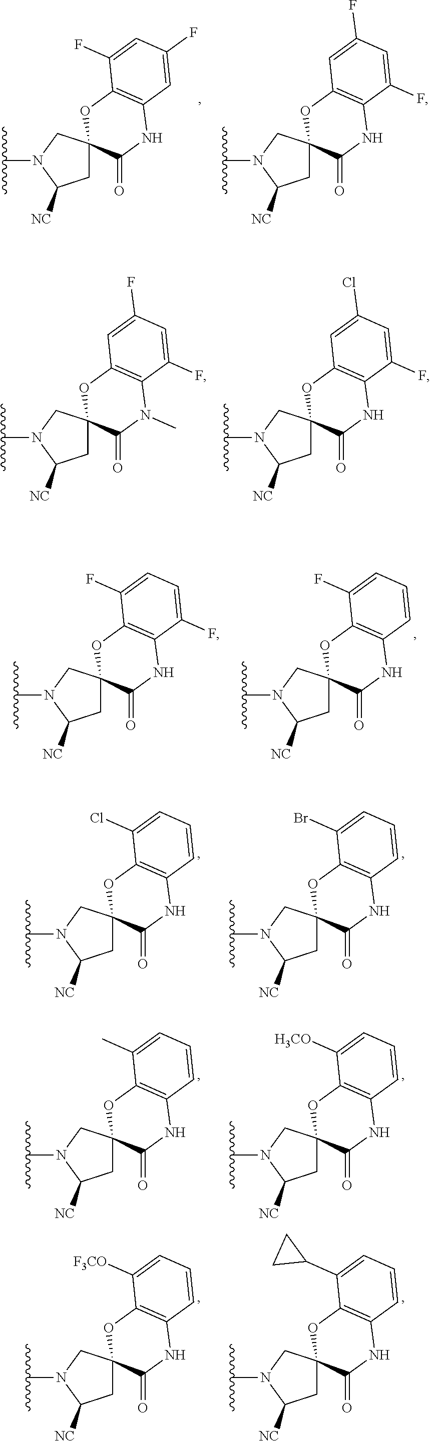

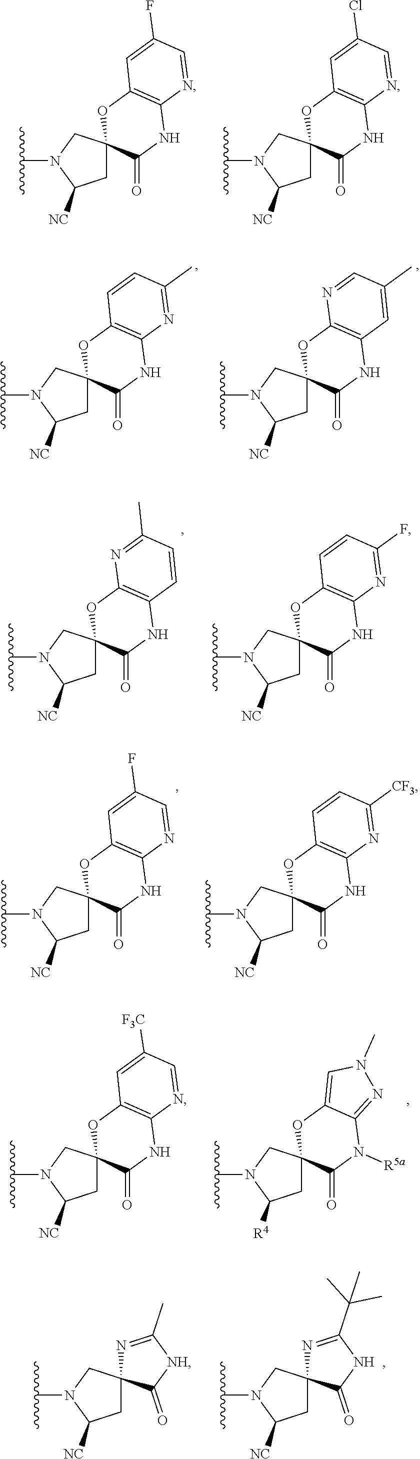

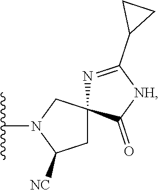 [SMILES: *N1C[C@@]2(C[C@H]1C#N)N=C(C1CC1)NC2=O]

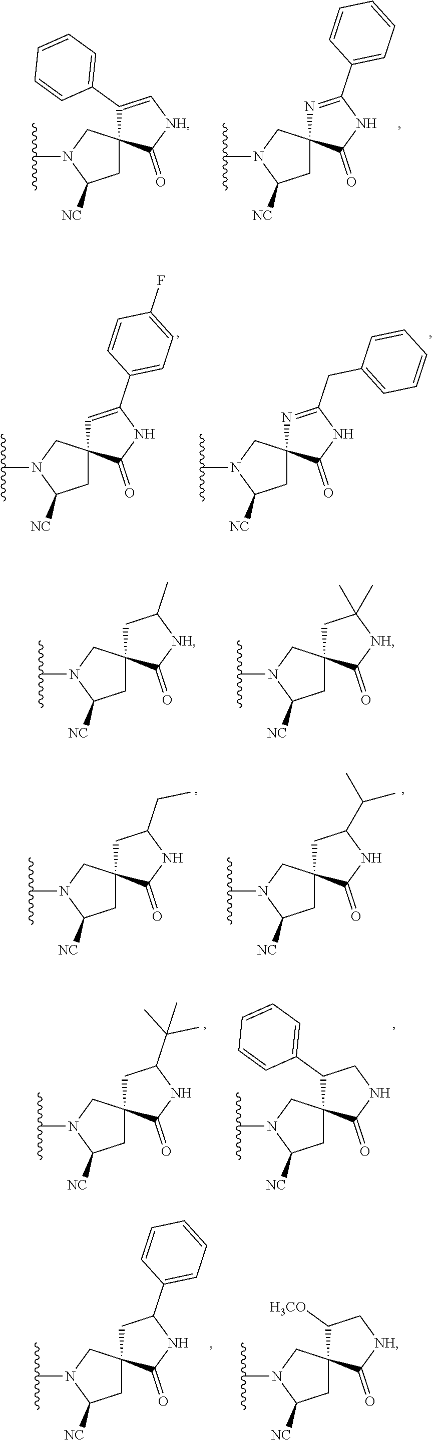

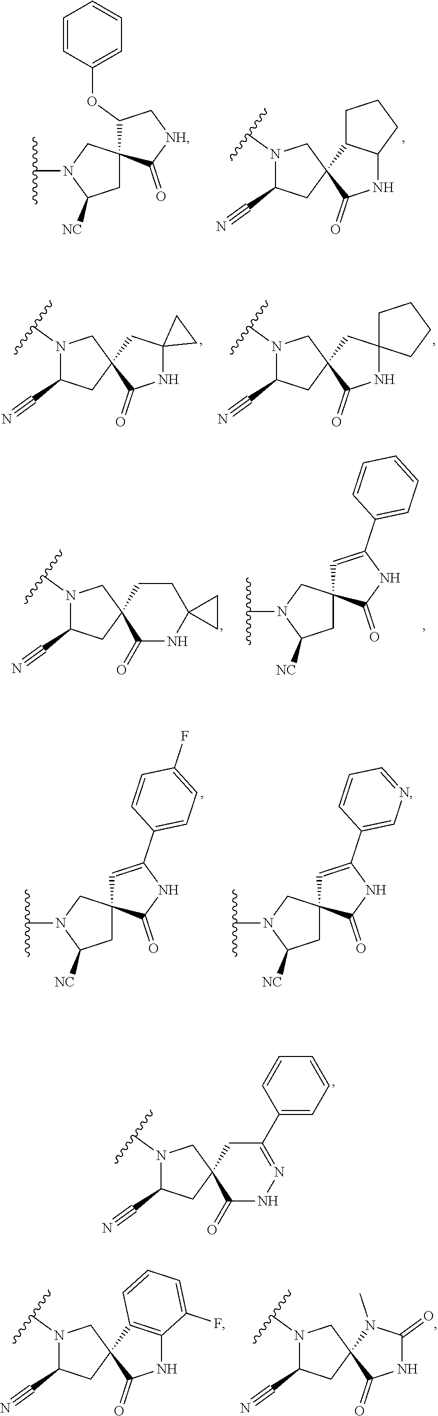

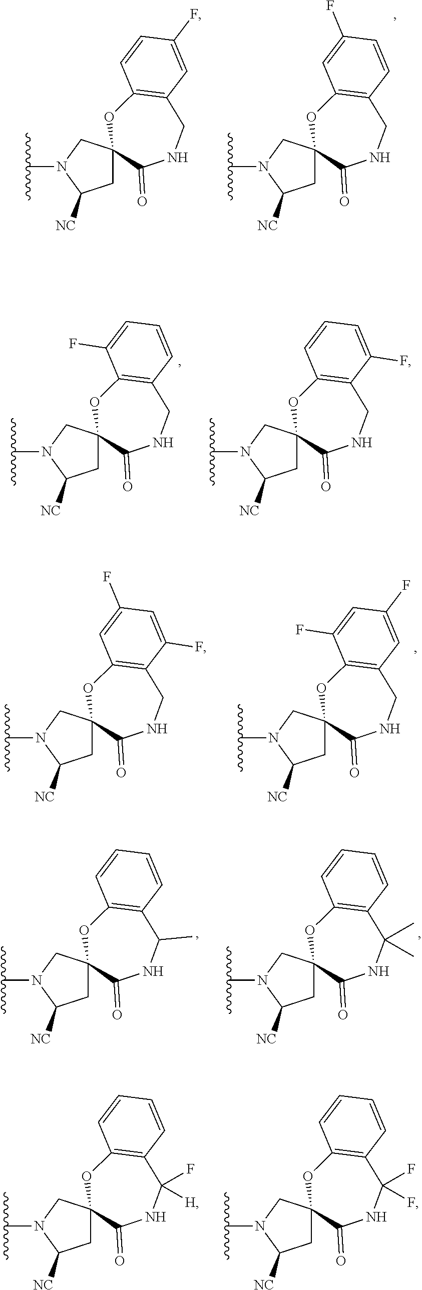

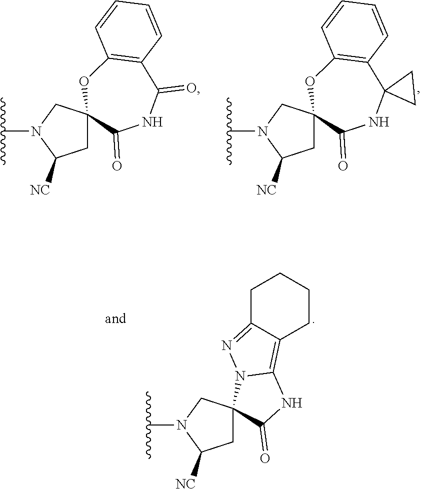 [SMILES: *N1C[C@@]2(C[C@H]1C#N)Oc1ccccc1C(=O)NC2=O ; *N1C[C@@]2(C[C@H]1C#N)Oc1ccccc1C1(CC1)NC2=O ; *N1C[C@]2(C[C@H]1C#N)C(=O)Nc1c3c(nn12)CCCC3]

In some embodiments, R¹ can be selected from

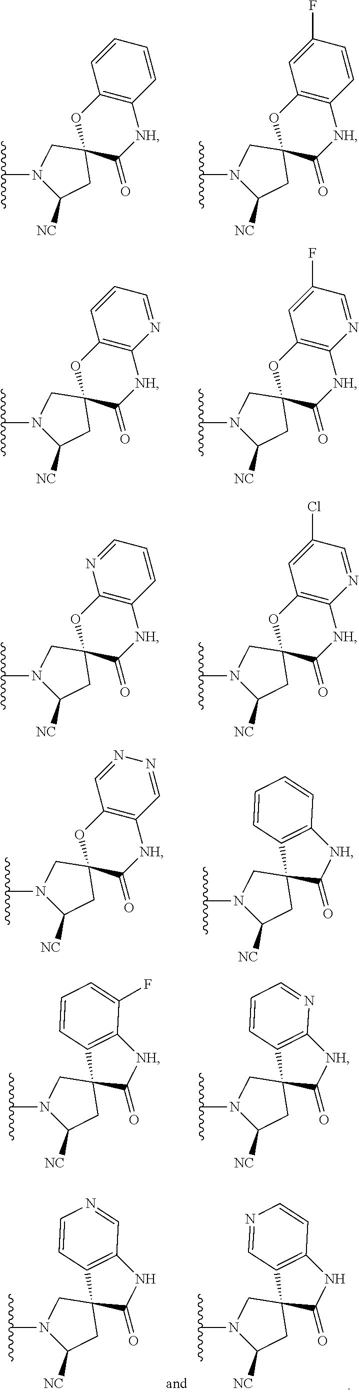

In some embodiments, R¹ can be

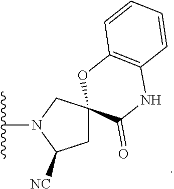 [SMILES: *N1C[C@@]2(C[C@H]1C#N)Oc1ccccc1NC2=O]

In other embodiments, R¹ can be

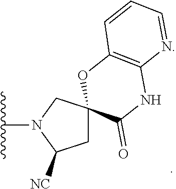 [SMILES: *N1C[C@@]2(C[C@H]1C#N)Oc1cccnc1NC2=O]

In still other embodiments, R¹ can be

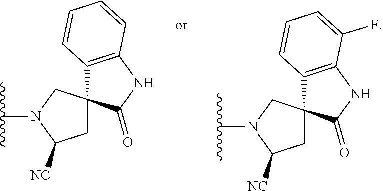 [SMILES: *N1C[C@]2(C[C@H]1C#N)C(=O)Nc1ccccc12 ; *N1C[C@]2(C[C@H]1C#N)C(=O)Nc1c(F)cccc12]

In yet still other embodiments, R¹ can be

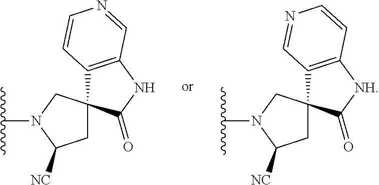 [SMILES: *N1C[C@]2(C[C@H]1C#N)C(=O)Nc1cnccc12 ; "or" ; *N1C[C@]2(C[C@H]1C#N)C(=O)Nc1ccncc12]

The substituent R⁴ can be various moieties. In some embodiments, R⁴ can be an unsubstituted ketoamide. In some embodiments, R⁴ can be a substituted ketoamide. The ketoamide can have the structure —C(═O)—C(═O)NR^{y1}R^{z1}. In some embodiments, R⁴ can be —C(═O)NH₂. In some embodiments, R⁴ can be an acyl, for example, R⁴ can be —C(═O)H, —C(═O)(an unsubstituted C₁₋₄ alkyl), —C(═O)(an unsubstituted or a substituted benzyl), —C(═O)(an unsubstituted or a substituted monocyclic heteroaryl) or —C(═O)(an unsubstituted or a substituted bicyclic heteroaryl). In some embodiments, R⁴ can be a substituted acyl. The acyl for R⁴ can have the structure —C(═O)R^{y2}. When the acyl is substituted, the possible groups that can be present on the acyl include hydroxy, a substituted or an unsubstituted alkoxy (such as —O-(an unsubstituted C₁₋₄ alkyl), —O-(an unsubstituted C₃₋₆ cycloalkyl), a substituted or an unsubstituted phenoxy or a substituted or an unsubstituted benzyloxy) or —O—(C=O)-(an unsubstituted C₁₋₆ alkyl).

R^{y1}, R^{y2} and R^{z1} can be a variety of groups. In some embodiments, R^{y1}, R^{y2} and R^{z1} can be independently selected from hydrogen, C₁₋₈ alkyl, C₂₋₈ alkenyl, C₂₋₈ alkynyl, C₃₋₈ cycloalkyl (for example, a monocyclic C₃₋₈ cycloalkyl), C₃₋₈ cycloalkenyl (such as a monocyclic C₃₋₈ cycloalkenyl), aryl (such as phenyl or naphthyl), heteroaryl (including a monocyclic or a bicyclic heteroaryl), heterocyclyl (for example, a monocyclic or a bicyclic heterocyclyl), aryl(alkyl) (such as benzyl), heteroaryl(alkyl) (including a monocyclic heteroaryl(CH₂)— and a monocyclic (heteroaryl(CH₂CH₂)—) or heterocyclyl(alkyl) (such as a monocyclic heterocyclyl(CH₂)— and a monocyclic heterocyclyl(CH₂CH₂)—), wherein each of the aforementioned R^{y1}, R^{y2} and R^{z1} groups can be unsubstituted or substituted. In some embodiments, R^{y1}, R^{y2} and R^{z1} can be independently selected from H, C₁₋₈ alkyl, an unsubstituted C₁₋₄ haloalkyl (including —CF₃, —CHF₂, —C(CH₃)F₂, —CH₂F, —CH(CH₃)F, —CH₂CF₃, —CH₂CH₂F and —CH₂CH₂CH₂F), —C₁₋₄ alkyl(OH) (including —CH₂OH, —CH₂CH₂OH and —CH(CH₃)OH), —C₁₋₄ alkyl(C₁₋₄ alkoxy) (such as —CH₂O(an unsubstituted C₁₋₄ alkyl) and —CH₂CH₂O(an unsubstituted C₁₋₄ alkyl)), —C₁₋₄ alkyl-O-(a monocyclic C₃₋₆ cycloalkyl) (such as —CH₂O(a monocyclic C₃₋₆ cycloalkyl), —CH₂CH₂O(a monocyclic C₃₋₆ cycloalkyl)), —C₁₋₄ alkyl-O-(phenyl) (for example, —CH₂O(phenyl) and —CH₂CH₂O(phenyl)), —C₁₋₄ alkyl-O-(5- to 6-membered monocyclic heteroaryl) (such as —CH₂O (5- to 6-membered monocyclic heteroaryl) and —CH₂CH₂O (5- to 6-membered monocyclic heteroaryl)), —C₁₋₄ alkyl-O-(5- to 6-membered monocyclic heterocyclyl) (for example, —CH₂O (5- to 6-membered monocyclic heterocyclyl) and —CH₂CH₂O (5- to 6-membered monocyclic heterocyclyl)), —C₁₋₄ alkyl-O-(a monocyclic C₃₋₆ cycloalkyl(C₁₋₄ alkyl) (such as —C₁₋₄ alkyl-O—CH₂-(monocyclic C₃₋₆ cycloalkyl) and —C₁₋₄ alkyl-O—CH₂CH₂-(monocyclic C₃₋₆ cycloalkyl)), —C₁₋₄ alkyl-O-(benzyl) (for example, —CH₂O(benzyl) and —CH₂CH₂O(benzyl)), —C₁₋₄ alkyl-O-(5- to 6-membered monocyclic heteroaryl(C₁₋₄ alkyl), —C₁₋₄ alkyl-O-(5- to 6-membered monocyclic heterocyclyl(C₁₋₄ alkyl), —C₁₋₄ alkyl-O(C═O)(an unsubstituted C₁₋₆ alkyl) (for example, —CH₂O(C═O)(an unsubstituted C₁₋₆ alkyl)), a monocyclic C₃₋₈ cycloalkyl (such as cyclopropyl, cyclobutyl, cyclopentyl, cyclohexyl, cycloheptyl and cyclooctyl), a monocyclic heteroaryl (such as imidazole, 1,3,4-oxadiazole and pyridinyl), a monocyclic heterocyclyl (for example, THF and tetrahydropyran), a bicyclic heteroaryl (for example, benzothiazole, benzoimidazole and benzooxazole), a bicyclic heterocyclyl, a monocyclic C₃₋₆ cycloalkyl(alkyl), aryl(alkyl) (such as benzyl), heteroaryl(alkyl) (for example, a monocyclic heteroaryl-(CH₂)—, such as pyridinyl-(CH₂)—) and heterocyclyl(alkyl) (for example, a monocyclic heterocyclyl-(CH₂)—), wherein each of the aforementioned R^{y1}, R^{y2} and R^{z1} groups can be unsubstituted or substituted.

In some embodiments, R⁴ can be —C(═O)R^{y2}, wherein R^{y2} can be —C₁₋₄ alkyl(OH) (such as —CH₂OH). In some embodiments, R⁴ can be —C(═O)—C(═O)NR^{y1}R^{z1}; wherein R^{yl} can be H; and R^{z1} can be any of the moieties listed for R^{z1} in the previous paragraph. In some embodiments, R⁴ can be —C(═O)—C(═O)NR^{y1}R^{z1}; wherein R^{yl} can be H; and R^{z1} can be a monocyclic C₃₋₈ cycloalkyl (for example, cyclopropyl, cyclobutyl, cyclopentyl, cyclohexyl, cycloheptyl and cyclooctyl).

Prodrug-type and phosphate-containing moieties can be present at R⁴. In some embodiments, R⁴ can be —CH(OH)—(S(═O)₂—OH). Those skilled in the art understand that when a salt is formed from —CH(OH)—(S(═O)₂—OH), R⁴ can be —CH(OH)—(S(═O)₂—O⁻) and the negative charge can be balanced with a positive ion to form a salt. For example, R⁴ can be —CH(OH)—(S(═O)₂—O⁻)(Na⁺). In other embodiments, R⁴ can be —CH(OH)((P=O)(OR⁶)₂), wherein each R⁶ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). In still other embodiments, R⁴ can be —C(═O)CH₂—O—((P=O)(OR⁷)₂), wherein each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). Other examples of R⁶ and R⁷ groups include methyl, ethyl, n-propyl, iso-propyl, n-butyl, iso-butyl, sec-butyl, tert-butyl, pentyl (straight-chained and branched), hexyl (straight-chained and branched), ethenyl, propenyl, butenyl, pentenyl, hexenyl, chloromethyl, fluoromethyl, difluoromethyl, dichloromethyl, trifluoromethyl, trichloromethyl, an unsubstituted or a substituted phenyl and an unsubstituted or a substituted benzyl.

In some embodiments, R⁴ can be cyano. In other embodiments, R⁴ can be an unsubstituted C₂₋₅ alkynyl. In still other embodiments, R⁴ can be a substituted C₂₋₅ alkynyl. The C₂₋₅ alkynyl can have various structures. For example, the C₂₋₅ alkynyl can have the structure —(CH₂)₁—C₂₋₄ alkynyl or —(CH₂)₂—C₂₋₃ alkynyl.

In some embodiments, R² can be an unsubstituted C₁₋₈ alkyl. For example, R² can be methyl, ethyl, n-propyl, iso-propyl, n-butyl, iso-butyl, sec-butyl, tert-butyl, pentyl (straight-chained and branched), hexyl (straight-chained and branched), heptyl (straight-chained and branched) and octyl (straight-chained and branched). In still other embodiments, R² can be a substituted C₁₋₈ alkyl. For example, R² can be a C₁₋₈ alkyl substituted with a monocyclic C₃₋₆ cycloalkyl (wherein the monocyclic C₃₋₆ cycloalkyl is unsubstituted or substituted), a C₁₋₈ alkyl substituted with one or more halogens (for example, 1, 2, 3, 4, 5 or 6 halogen) or a C₁₋₈ alkyl substituted with a —O-(an unsubstituted C₁₋₄ alkyl). The monocyclic C₃₋₆ cycloalkyl that can be substituted on a C₁₋₈ alkyl can be cyclopropyl, cyclobutyl, cyclopentyl or cyclohexyl. In some embodiments, R² can be —(CH₂)-(an unsubstituted or a substituted monocyclic C₃₋₆ cycloalkyl). When the C₃₋₆ cycloalkyl that is substituted on a C₁_8 alkyl is substituted, one or more other moieties can be present. For example, 1, 2, 3 or 4 moieties can be present on a C₃₋₆ cycloalkyl that is substituted on a C₁₋₈ alkyl for R². In some embodiments, R² can be a C₁₋₄ alkyl substituted with a cyclopropyl, cyclobutyl or a cyclohexyl (wherein the cyclopropyl, cyclobutyl and/or cyclohexyl can be unsubstituted or substituted with 1 or 2 halogens and/or 1 or 2 unsubstituted C₁₋₄ alkyls). In other embodiments, R² can be a C₁₋₄ alkyl substituted with 1, 2, 3, 4, 5 or 6 fluoros.

As described herein, R² can be an unsaturated hydrocarbon. In some embodiments, R² can be an unsubstituted C₂₋₈ alkenyl. In other embodiments, R² can be a substituted C₂₋₈ alkenyl. In still other embodiments, R² can be an unsubstituted C₂₋₈ alkynyl. In yet still other embodiments, R² can be a substituted C₂₋₈ alkynyl.

A variety of cyclic groups can be present for R² where the cyclic group can be a hydrocarbon cyclic group or a cyclic group that include 1, 2, 3 or 4 heteroatoms (such as N (nitrogen), O (oxygen) and S (sulfur)). In some embodiments, R² can be an unsubstituted C₃₋₁₀ cycloalkyl. In other embodiments, R² can be a substituted C₃₋₁₀ cycloalkyl. The C₃₋₁₀ cycloalkyl can be a monocyclic C₃₋₁₀ cycloalkyl or a multicyclic C₃₋₁₀ cycloalkyl (for example, a bicyclic or tricyclic C₃₋₁₀ cycloalkyl). In other embodiments, R² can be an unsubstituted C₃₋₁₀ cycloalkenyl. In still other embodiments, R² can be a substituted C₃₋₁₀ cycloalkenyl. As with the C₃₋₁₀ cycloalkyl, the C₃₋₁₀ cycloalkenyl can be monocyclic or multicyclic.

In some embodiments, R² can be an unsubstituted aryl. In other embodiments, R² can be a substituted aryl. In still other embodiments, R² can be an unsubstituted heteroaryl. In yet still other embodiments, R² can be a substituted heteroaryl. In some embodiments, R² can be an unsubstituted heterocyclyl. In other embodiments, R² can be a substituted heterocyclyl. The aryl, heteroaryl and heterocyclyl for R² can be monocyclic or multicyclic (for example, bicyclic or tricyclic).

The cyclic moiety that can be present for R² can be connected via a C₁₋₄ alkylene linker. For example, the cyclic moiety for R² can be connected via a methylene linker. In some embodiments, R² can be an unsubstituted aryl(alkyl). In other embodiments, R² can be a substituted aryl(alkyl). In still other embodiments, R² can be an unsubstituted heteroaryl(alkyl). In yet still other embodiments, R² can be a substituted heteroaryl(alkyl). In some embodiments, R² can be an unsubstituted heterocyclyl(alkyl). In other embodiments, R² can be a substituted heterocyclyl(alkyl).

As described herein, the cyclic moiety for R² can be monocyclic or multicyclic (for example, bicyclic or tricyclic). When R² includes a monocyclic aryl, R² can include an unsubstituted or a substituted phenyl. In some embodiments, R² can be an unsubstituted phenyl. In other embodiments, R² can be a substituted phenyl. In still other embodiments, R² can be an unsubstituted benzyl. In yet still other embodiments, R² can be a substituted benzyl. For example, the benzyl can be substituted with 1, 2, 3 or 4 halogens, such as fluoro and/or chloro, and/or cyano.

When R² includes a heteroaryl or a heterocyclyl (such as when R² is a heteroaryl, a heterocyclyl, a heteroaryl(alkyl) or a heterocyclyl(alkyl)), 1, 2 or 3 heteroatoms can be present in the ring(s). In some embodiments, the heterocyclyl that can be included in R² can be a 3- to 10-membered heterocyclyl. Examples of suitable heteroatoms include N (nitrogen), O (oxygen) and S (sulfur). In some embodiments, R² can include a monocyclic heteroaryl (such as a 5- or 6-membered heteroaryl). In other embodiments, R² can include a monocyclic heterocyclyl (such as a 5- or 6-membered heterocyclyl). In still other embodiments, R² can include a bicyclic heteroaryl (such as a 9- or 10-membered heteroaryl). In other embodiments, R² can include a bicyclic heterocyclyl (such as a 9- or 10-membered heterocyclyl). Examples or suitable heteroaryls and heterocyclyls include furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine, pyrazine, azetidine, oxetane, thietane, tetrahydrofuran, tetrahydrothiophene, pyrrolidine, oxazolidin-2-one, tetrahydropyran, tetrahydrothiopyran, piperidine, piperazine, morpholine and thiomorpholine.

A non-limiting list of R² groups include:

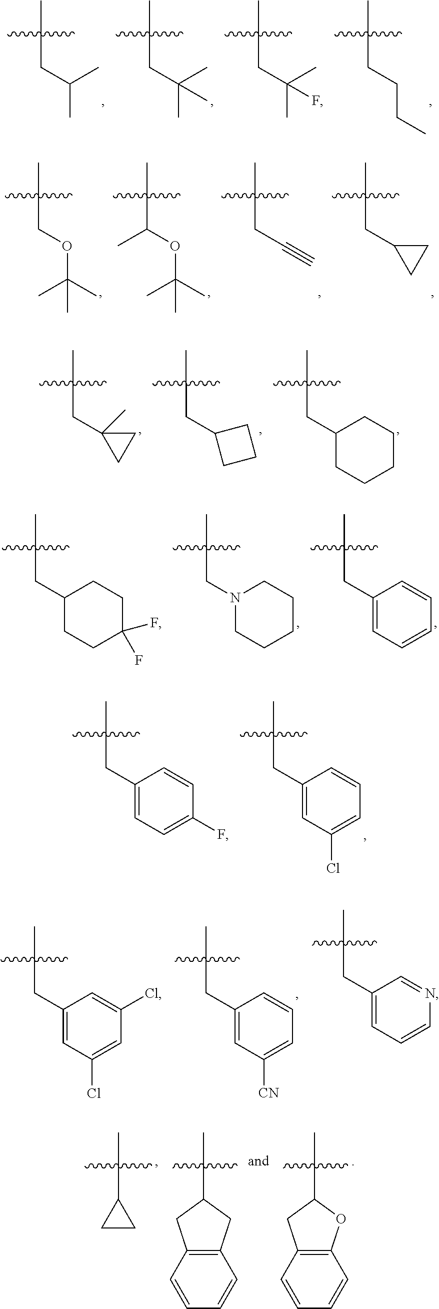

In some embodiments, R² can be

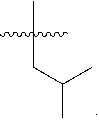 [SMILES: *CC(C)C]

In other embodiments, R² can be

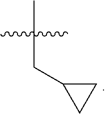 [SMILES: *CC1CC1]

The carbon to which R² is attached can be a stereocenter. In some embodiments, the carbon to which R² is attached can be in the (S)-configuration. In other embodiments, the carbon to which R² is attached can be in the (R)-configuration. The stereo-version of a compound of Formula (I), or a pharmaceutically acceptable salt thereof, is provided below:

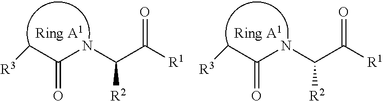

As provided herein R³ can be

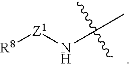

In some embodiments, R³ can be

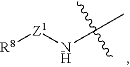

wherein Z¹ is —C(═O)— such that R³ can be

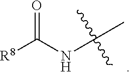

In some embodiments, R³ can be

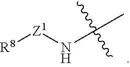

wherein Z¹ is —S(═O)₂— such that R³ can be

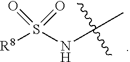

In some embodiments, R⁸ can be an unsubstituted C₁₋₆ haloalkyl. For example, R⁸ can be —CF₃, —CClF₂, —CFCl₂, —CHF₂, —C(CH₃)F₂, —CH₂F, —CH(CH₃)F, —C(CH₃)F₂, —CF(CH₃)₂, —CH₂CF₃, —CH(CH₃)CF₃, —CH₂CH₂CF₃, —CH₂CH(CH₃)CF₃, —CF₂CF₃, —CH₂CH₂F, —CF₂CF₂CF₃ and —CH₂CH₂CH₂F. In some embodiments, R⁸ can be —CF₃. In other embodiments, R⁸ can be a substituted C₁₋₆ haloalkyl where the C₁₋₆ haloalkyl can be substituted 1 or 2 times with an unsubstituted C₁₋₄ alkoxy (such as —O-(an unsubstituted C₁₋₄ alkyl)). When the C₁₋₆ haloalkyl is substituted with 1 or 2 unsubstituted C₁₋₄ alkoxys, one or more hydrogens of the C₁₋₆ haloalkyl (for example, 1, 2 or 3 hydrogens) can be replaced with an unsubstituted C₁₋₄ alkoxy (for example —O(C₁₋₄ alkyl) such as methoxy, ethoxy, n-propoxy, isopropoxy, n-butoxy, iso-butoxy, sec-butoxy and tert-butoxy). Exemplary C₁₋₆ haloalkyls substituted with an unsubstituted C₁₋₄ alkoxy include —C(OCH₃)F₂, —CH(OCH₃)F, —C(OCH₃)(CH₃)F, —CH(OCH₃)CF₃, —C(OCH₃)(CH₃)CF₃, —CH₂CH(OCH₃)CF₃, —CH₂C(OCH₃)(CH₃)CF₃, —CH₂CH(OCH₃)F and —CH₂CH₂CH(OCH₃)F. In still other embodiments, R⁸ can be a substituted C₁₋₆ haloalkyl where the C₁₋₆ haloalkyl can be substituted 1 or 2 times with an unsubstituted or a substituted C₃₋₆ cycloalkyl (such as C₁₋₆ haloalkyl substituted with an unsubstituted monocyclic C₃₋₆ cycloalkyl, C₁₋₆ haloalkyl substituted with a substituted monocyclic C₃₋₆ cycloalkyl, C₁₋₆ haloalkyl substituted with an unsubstituted bicyclic C₃₋₆ cycloalkyl and C₁₋₆ haloalkyl substituted with a substituted bicyclic C₃₋₆ cycloalkyl). An example of a substituted C₁₋₆ haloalkyl where the C₁₋₆ haloalkyl can be substituted 1 or 2 times with an unsubstituted or a substituted C₃₋₆ cycloalkyl is —CF₂(cyclopropyl). When the C₃₋₆ cycloalkyl is substituted, the C₃₋₆ cycloalkyl can be substituted with a variety of substituents. For example, the C₃₋₆ cycloalkyl can be substituted with a halogen, an unsubstituted C₁₋₄ alkyl and/or an unsubstituted C₁₋₄ haloalkyl (such as —CF₃, —CHF₂ and/or —CH₂F).

In some embodiments, R⁸ can be an unsubstituted C₁₋₆ alkyl, such as methyl, ethyl, n-propyl, iso-propyl, n-butyl, iso-butyl, sec-butyl, tert-butyl, pentyl (straight-chained or branched) and hexyl (straight-chained or branched). In other embodiments, R⁸ can be a C₁₋₆ alkyl substituted 1 or 2 times with an unsubstituted C₁₋₄ alkoxy (such as a —O-(an unsubstituted C₁₋₄ alkyl)) and/or hydroxy. When the C₁₋₆ alkyl is substituted with an unsubstituted C₁₋₄ alkoxy and/or hydroxy, a hydrogen of the C₁₋₆ alkyl can be replaced with an unsubstituted C₁₋₄ alkoxy and/or hydroxy such as those described herein. A non-limiting list of C₁₋₆ alkyls substituted 1 or 2 times with an unsubstituted C₁₋₄ alkoxy include —CH(OH)(CH₃)₃, —CH₂(OCH₃), —CH(OCH₃)₂, —CH(CH₃)(OCH₃) and —C(CH₃)₂(OCH₃). In still other embodiments, R⁸ can be a C₁₋₆ alkyl substituted with 1 to 13 times with deuterium. For example, R⁸ can be —CD₃ or -CD₂CD₃.

In some embodiments, R⁸ can be an unsubstituted phenyl. In some embodiments, R⁸ can be a substituted phenyl. When the phenyl is substituted, a variety of substituents can be present, and the number of substituents can vary. In some embodiments, R⁸ can be a phenyl substituted 1, 2, 3 or 4 times with a moiety independently selected from halogen, an unsubstituted C₁₋₆ alkyl, an unsubstituted C₁₋₆ haloalkyl, an unsubstituted C₁₋₆ alkoxy (for example, —O-(an unsubstituted C₁₋₄ alkyl)) and an unsubstituted or a substituted monocyclic heteroaryl. For example, R⁸ can be a phenyl substituted 1, 2, 3 or 4 times with a moiety independently selected from F, Cl, Br, as methyl, ethyl, n-propyl, iso-propyl, n-butyl, iso-butyl, sec-butyl, tert-butyl, pentyl (straight-chained or branched), hexyl (straight-chained or branched), —CF₃, —CHF₂, —C(CH₃)F₂, —CH₂F, —CH(CH₃)F, —CH₂CF₃, —CH₂CH₂F, —CH₂CH₂CH₂F, —O(an unsubstituted C₁₋₆ alkyl) (such as methoxy, ethoxy, n-propoxy, iso-propoxy, n-butoxy, iso-butoxy, sec-butoxy, tert-butoxy, pentoxy (straight-chained or branched), hexoxy (straight-chained or branched)), and an unsubstituted or a substituted 5- to 6-membered monocyclic heteroaryl, wherein the heteroaryl can include 1, 2 or 3 heteroatoms selected from oxygen, sulfur and nitrogen. Exemplary monocyclic heteroaryls include furan, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine.

In some embodiments, R⁸ can be an unsubstituted or a substituted monocyclic heteroaryl. A variety of an unsubstituted or a substituted monocyclic heteroaryls can be present for R⁸. For example, the heteroaryl can be a 5- or 6-membered heteroaryl that includes 1, 2 or 3 heteroatoms selected from nitrogen (N), oxygen (O) and sulfur (S). Exemplary heteroaryls for an unsubstituted or a substituted monocyclic heteroaryl include, but are not limited to, furan, isoxazole, isothiazole, pyrrole, pyrazole, oxazole, thiazole, imidazole, 1,2,3-triazole, 1,2,4-triazole, imidazole, 1,3,4-oxadiazole, 1,3,4-thiadiazole, pyridine, pyridazine, pyrimidine, pyridazine and pyrazine. In yet still other embodiments, R⁸ can be an unsubstituted or a substituted monocyclic heterocyclyl. A non-limiting list of monocyclic heterocyclyls for R⁸ include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. Various substituents can be present on a substituted heteroaryl and/or a substituted heterocyclyl of R⁸. For example, the heteroaryl and/or heterocyclyl of R⁸ can be substituted 1, 2 or 3 times with a moiety selected from halogen, an unsubstituted C₁₋₆ alkyl, an unsubstituted C₁₋₆ haloalkyl, an unsubstituted C₁₋₆ alkoxy (e.g., —O-(an unsubstituted C₁₋₄ alkyl)) and an unsubstituted or a substituted phenyl. Suitable halogens, unsubstituted C₁₋₆ alkyls, unsubstituted C₁₋₆ haloalkyls and unsubstituted C₁₋₆ alkoxys are described herein, including those that can be present on a phenyl of R⁸. An unsubstituted or a substituted phenyl that can be substituted on a heteroaryl or a heterocyclyl of R⁸ can substituted 1, 2, 3, 4 or 5 times. A non-limiting list of examples of substituents that can be substituted on a phenyl that is substituted on a heteroaryl or heterocyclyl of R⁸ include F, Cl, Br, as methyl, ethyl, n-propyl, iso-propyl, n-butyl, iso-butyl, sec-butyl, tert-butyl, pentyl (straight-chained or branched), hexyl (straight-chained or branched), —CF₃, —CHF₂, —C(CH₃)F₂, —CH₂F, —CH(CH₃)F, —CH₂CF₃, —CH₂CH₂F, —CH₂CH₂CH₂F, —O(an unsubstituted C₁₋₆ alkyl) (such as methoxy, ethoxy, n-propoxy, iso-propoxy, n-butoxy, iso-butoxy, sec-butoxy, tert-butoxy, pentoxy (straight-chained or branched), hexoxy (straight-chained or branched)), and an unsubstituted or a substituted 5- to 6-membered monocyclic heteroaryl, wherein the heteroaryl can include 1, 2 or 3 heteroatoms selected from oxygen, sulfur and nitrogen. Examples of monocyclic heteroaryls include furan, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine.

In some embodiments, R⁸ can be an unsubstituted monocyclic C₃₋₆ cycloalkyl, such as cyclopropyl, cyclobutyl, cyclopentyl and cyclohexyl. In other embodiments, R⁸ can be a substituted monocyclic C₃₋₆ cycloalkyl, wherein the monocyclic C₃₋₆ cycloalkyl is substituted 1, 2, 3, 4, 5 or 6 times). In some embodiments, R⁸ can be a halogen-substituted monocyclic C₃₋₆ cycloalkyl (for example, a monocyclic C₃₋₆ cycloalkyl substituted 1, 2, 3 or 4 times with a halogen such as fluoro). In still other embodiments, R⁸ can be a monocyclic C₃₋₆ cycloalkyl substituted with an unsubstituted C₁₋₄ alkyl. In yet still other embodiments, R⁸ can be a monocyclic C₃₋₆ cycloalkyl substituted with an unsubstituted C₁₋₄ alkoxy (e.g., —O-(an unsubstituted C₁₋₄ alkyl)). In some embodiments, R⁸ can be a monocyclic C₃₋₆ cycloalkyl substituted with an unsubstituted C₂₋₄ alkenyl. In other embodiments, R⁸ can be a monocyclic C₃₋₆ cycloalkyl substituted with an unsubstituted C₁₋₄ haloalkyl (such as —CF₃, —CHF₂ and/or —CH₂F). In still other embodiments, R⁸ can be a monocyclic C₃₋₆ cycloalkyl substituted with an unsubstituted monocyclic C₃₋₆ cycloalkyl, such as cyclopropyl, cyclobutyl, cyclopentyl or cyclohexyl. In some embodiments, R⁸ can be an unsubstituted bicyclic C₅₋₆ cycloalkyl. In other embodiments, R⁸ can be a substituted bicyclic C₅₋₆ cycloalkyl. The two rings of a bicyclic C₅₋₆ cycloalkyl can be connected in a spiro-fashion, a fused-fashion or a bridged-fashion. In some embodiments, R⁸ can be a halogen-substituted bicyclic C₅₋₆ cycloalkyl. In still other embodiments, R⁸ can be a bicyclic C₅₋₆ cycloalkyl substituted with an unsubstituted C₁₋₄ alkyl. In yet still other embodiments, R⁸ can be a bicyclic C₅₋₆ cycloalkyl substituted with an unsubstituted C₁₋₄ alkoxy (such as —O-(an unsubstituted C₁₋₄ alkyl)). In some embodiments, R⁸ can be a bicyclic C₅₋₆ cycloalkyl substituted with an unsubstituted C₂₋₄ alkenyl. In other embodiments, R⁸ can be a bicyclic C₅₋₆ cycloalkyl substituted with an unsubstituted C₁₋₄ haloalkyl. In still other embodiments, R⁸ can be a bicyclic C₅₋₆ cycloalkyl substituted with an unsubstituted monocyclic C₃₋₆ cycloalkyl (including cyclopropyl, cyclobutyl, cyclopentyl and cyclohexyl). A non-liming list of bicyclic C₅₋₆ cycloalkyls include spiro[2.2]pentane, spiro[2.3]hexane, bicyclo[1.1.1]pentane and bicyclo[2.1.1]hexane.

Suitable halogen-substituted monocyclic C₃₋₆ cycloalkyls include halogen-substituted cyclopropyl, halogen-substituted cyclobutyl, halogen-substituted cyclopentyl and halogen-substituted cyclohexyl. Additional monocyclic C₃₋₆ cycloalkyls include cyclopropyl substituted with an unsubstituted C₁₋₄ alkyl, an unsubstituted C₂₋₄ alkenyl, an unsubstituted C₁₋₄ alkoxy (for example, —O-(an unsubstituted C₁₋₄ alkyl)), an unsubstituted C₁₋₄ haloalkyl and/or an unsubstituted monocyclic C₃₋₆ cycloalkyl, cyclobutyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl, cyclopentyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl and cyclohexyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl. The number halogens on a halogen-substituted monocyclic C₃₋₆ cycloalkyl and/or a bicyclic C₅₋₆ cycloalkyl, the number of unsubstituted C₁₋₄ alkyls on a monocyclic C₃₋₆ cycloalkyl and/or a bicyclic C₅₋₆ cycloalkyl, the number of unsubstituted C₁₋₄ alkoxys on a monocyclic C₃₋₆ cycloalkyl and/or a bicyclic C₅₋₆ cycloalkyl, the number of unsubstituted C₂₋₄ alkenyls on a monocyclic C₃₋₆ cycloalkyl and/or a bicyclic C₅₋₆ cycloalkyl, the number of unsubstituted C₁₋₄ haloalkyls on a monocyclic C₃₋₆ cycloalkyl and/or a bicyclic C₅₋₆ cycloalkyl and the number of unsubstituted monocyclic C₃₋₆ cycloalkyls on a monocyclic C₃₋₆ cycloalkyl and/or a bicyclic C₅₋₆ cycloalkyl can vary. For example, 1, 2, 3 or 4 halogens can be present on a halogen-substituted monocyclic C₃₋₆ cycloalkyl, 1, 2, 3 or 4 unsubstituted C₁₋₄ alkyls can be present on a monocyclic C₃₋₆ cycloalkyl substituted with an unsubstituted C₁₋₄ alkyl, 1, 2, 3 or 4 unsubstituted C₁₋₄ alkoxys can be present on a monocyclic C₃₋₆ cycloalkyl substituted with an unsubstituted C₁₋₄ alkoxy, 1, 2, 3 or 4 unsubstituted C₂₋₄ alkenyls can be present on a monocyclic C₃₋₆ cycloalkyl substituted with an unsubstituted C₂₋₄ alkenyl, 1, 2, 3 or 4 unsubstituted C₁₋₄ haloalkyls can be present on a monocyclic C₃₋₆ cycloalkyl substituted with an unsubstituted C₁₋₄ haloalkyl, 1 or 2 unsubstituted monocyclic C₃₋₆ cycloalkyls can be present on a monocyclic C₃₋₆ cycloalkyl, 1, 2, 3 or 4 halogens can be present on a halogen-substituted bicyclic C₅₋₆ cycloalkyl, 1, 2, 3 or 4 unsubstituted C₁₋₄ alkyls can be present on a bicyclic C₅₋₆ cycloalkyl substituted with an unsubstituted C₁₋₄ alkyl, 1, 2, 3 or 4 unsubstituted C₁₋₄ alkoxys can be present on a bicyclic C₅₋₆ cycloalkyl substituted with an unsubstituted C₁₋₄ alkoxy, 1, 2, 3 or 4 unsubstituted C₂₋₄ alkenyls can be present on a bicyclic C₅₋₆ cycloalkyl substituted with an unsubstituted C₂₋₄ alkenyl, 1, 2, 3 or 4 unsubstituted C₁₋₄ haloalkyls can be present on a bicyclic C₅₋₆ cycloalkyl substituted with an unsubstituted C₁₋₄ haloalkyl and 1 or 2 unsubstituted monocyclic C₃₋₆ cycloalkyls can be present on a bicyclic C₅₋₆ cycloalkyl. In some embodiments, a monocyclic C₃₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy, an unsubstituted C₂₋₄ alkenyl, and an unsubstituted C₁₋₄ haloalkyl. In other embodiments, a bicyclic C₅₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy an unsubstituted C₂₋₄ alkenyl, and an unsubstituted C₁₋₄ haloalkyl. Suitable halogens that can be present on a substituted monocyclic C₃₋₆ cycloalkyl include, but are not limited to, fluoro (F) and chloro (Cl). Examples of unsubstituted C₁₋₄ haloalkyls include, but are not limited to, —CF₃, —CHF₂, —C(CH₃)F₂, —CH₂F, —CH(CH₃)F, —CH₂CF₃, —CH₂CH₂F and —CH₂CH₂CH₂F.

In some embodiments, R⁸ can be an unsubstituted alkoxy. In other embodiments, R⁸ can be a substituted alkoxy. Various alkoxys can be present for R⁸. For example, —O-(hydrocarbon) (such as —O—(C₁₋₈ alkyl)), —O-(monocyclic C₃₋₈ cycloalkyl), —O-(bicyclic C₅₋₈ cycloalkyl), —O-(phenyl) and —O-(bicyclic aryl)),—O-(monocyclic heteroaryl), —O-(bicyclic heteroaryl), —O-(monocyclic heterocyclyl) and —O-(bicyclic heterocyclyl). A non-limiting list of examples of C₁₋₆ alkoxys are methoxy, ethoxy, n-propoxy, iso-propoxy, n-butoxy, iso-butoxy, sec-butoxy, tert-butoxy, pentoxy (straight-chained or branched), hexoxy (straight-chained or branched), —O-cyclopropyl, —O-cyclobutyl, —O-cyclopentyl, —O— cyclohexyl and —O-(bicyclo[1.1.1]pentyl). In some embodiments, R⁸ can be —O-(an unsubstituted or a substituted C₁₋₈ alkyl). In some embodiments, R⁸ can be —O-(an unsubstituted C₁₋₄ alkyl). For example, R⁸ can be —O—CH₃, —O—CH₂CH₃, —O—CH₂CH₂CH₃, —O—CH(CH₃)₂, —O—CH₂CH₂CH₂CH₃, —O—CH(CH₃)CH₂CH₃, —O—CH₂CH(CH₃)₂ and —O—C(CH₃)₃. A variety of substituents can be present on a substituted alkoxy for R⁸. Examples of suitable substituents are those provided for “optionally substituted.” In some embodiments, 1, 2, 3 or 4 substituents can be present on a substituted alkoxy. For example, a substituted alkoxy can be substituted 1, 2, 3 or 4 times with substituents independently selected from halogen (for example, F or Cl), hydroxy, an unsubstituted C₁₋₄ alkyl and an unsubstituted C₁₋₄ haloalkyl. In some embodiments, R⁸ can be an unsubstituted or substituted alkoxy selected from methoxy, ethoxy, n-propoxy, iso-propoxy, n-butoxy, iso-butoxy, sec-butoxy, tert-butoxy, —O— cyclopropyl, —O-cyclobutyl, —O-cyclopentyl, —O-cyclohexyl and —O-(bicyclo[1.1.1]pentyl), —O-(phenyl) and —O-(halo-substituted phenyl).

In some embodiments, R⁸ can be an amino or an amine, such as —NR¹⁴R¹⁵, wherein R¹⁴ and R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl). In other embodiments, R⁸ can be —NR¹⁴R¹⁵, wherein R¹⁴ and R¹⁵ are taken together along with the nitrogen to which they are connected to form an unsubstituted or a substituted 3-8 membered heterocyclyl.

In some embodiments, R¹⁴ and/or R¹⁵ can be an unsubstituted C₁₋₈ alkyl. In other embodiments, R¹⁴ and/or R¹⁵ can be a substituted C₁₋₈ alkyl. In still other embodiments, R¹⁴ and/or R¹⁵ can be an unsubstituted C₂₋₈ alkenyl. In yet still other embodiments, R¹⁴ and/or R¹⁵ can be a substituted C₂₋₈ alkenyl. In some embodiments, R¹⁴ and/or R¹⁵ can be an unsubstituted C₂₋₈ alkynyl. In other embodiments, R¹⁴ and/or R¹⁵ can be a substituted C₂₋₈ alkynyl. In still other embodiments, R¹⁴ and/or R¹⁵ can be an unsubstituted C₃₋₈ cycloalkyl, for example an unsubstituted monocyclic C₃₋₈ cycloalkyl. In yet still other embodiments, R¹⁴ and/or R¹⁵ can be a substituted C₃₋₈ cycloalkyl, for example a substituted monocyclic C₃₋₈ cycloalkyl. Various cyclic moieties can be present for R¹⁴ and/or R¹⁵. In some embodiments, R¹⁴ and/or R¹⁵ can be an unsubstituted aryl. In other embodiments, R¹⁴ and/or R¹⁵ can be a substituted aryl. In still other embodiments, R¹⁴ and/or R¹⁵ can be an unsubstituted heteroaryl. In yet still other embodiments, R¹⁴ and/or R¹⁵ can be a substituted heteroaryl. In some embodiments, R¹⁴ and/or R¹⁵ can be an unsubstituted 3- to 8-membered monocyclic heterocyclyl. In other embodiments, R¹⁴ and/or R¹⁵ can be a substituted 3- to 8-membered monocyclic heterocyclyl. In still other embodiments, R¹⁴ and/or R¹⁵ can be an unsubstituted aryl(alkyl). In yet still other embodiments, R¹⁴ and/or R¹⁵ can be a substituted aryl(alkyl). In some embodiments, R¹⁴ and/or R¹⁵ can be an unsubstituted heteroaryl(alkyl). In other embodiments, R¹⁴ and/or R¹⁵ can be a substituted heteroaryl(alkyl). The aryl, heteroaryl and heterocyclyl can be monocyclic or bicyclic, and include 1, 2, 3, 4 or 5 heteroatoms independently selected from O (oxygen), S (sulfur) and N (nitrogen). When R¹⁷ and/or R¹⁵ is aryl(alkyl) or heteroaryl(alkyl), the alkyl linker can be 1, 2 or 3 alkylene groups, such as —CH₂—CH₂CH₂— and —CH₂CH₂CH₂—. In some embodiments, R⁸ can be —NHR¹⁵, wherein R¹⁸ can be as provided herein. For example, in some embodiments, R⁸ can be —NHR¹⁵, wherein R¹⁸ can be an unsubstituted C₁₋₈ alkyl.

In some embodiments, R³ can be R⁹. As described herein, R⁹ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted 5- to 8-membered bicyclic heterocyclyl, an unsubstituted or a substituted aryl(alkyl), an unsubstituted or a substituted heteroaryl(alkyl), an unsubstituted or a substituted C-carboxy, —OR¹⁰, —NR¹¹R¹² or —C(═O)—NR^{13A}R^{13B}. In some embodiments, R⁹ can be an unsubstituted C₁₋₈ alkyl. In other embodiments, R⁹ can be a substituted C₁₋₈ alkyl. For example R⁹ can be a C₁₋₈ alkyl substituted with a mono-substituted amine (such as C₁₋₈ alkyl substituted with —NHR^{9A}, wherein R^{9A} can be an unsubstituted or substituted cyclic moiety, including an unsubstituted or substituted aryls, an unsubstituted or substituted heteroaryls and an unsubstituted or substituted heterocyclyls). In still other embodiments, R⁹ can be an unsubstituted C₂₋₈ alkenyl. In yet still other embodiments, R⁹ can be a substituted C₂₋₈ alkenyl. In some embodiments, R⁹ can be an unsubstituted C₂₋₈ alkynyl. In other embodiments, R⁹ can be a substituted C₂₋₈ alkynyl.

A variety of cyclic moieties can be present for R⁹. In some embodiments, R⁹ can be an unsubstituted C₃₋₈ cycloalkyl. In other embodiments, R⁹ can be a substituted C₃₋₈ cycloalkyl. For example, R⁹ can be an unsubstituted or a substituted monocyclic C₃₋₈ cycloalkyl. In still other embodiments, R⁹ can be an unsubstituted aryl. In yet still other embodiments, R⁹ can be a substituted aryl. As an example, R⁹ can be an unsubstituted or a substituted phenyl. In some embodiments, R⁹ can be an unsubstituted heteroaryl. In other embodiments, R⁹ can be a substituted heteroaryl. In some embodiments, R⁹ can be an unsubstituted 3- to 12-membered monocyclic heterocyclyl. In other embodiments, R⁹ can be a substituted 3- to 12-membered monocyclic heterocyclyl. In still other embodiments, R⁹ can be an unsubstituted or a substituted 5- to 12-membered bicyclic heterocyclyl. In yet still other embodiments, R⁹ can be an unsubstituted or a substituted 5- to 12-membered bicyclic heterocyclyl. In some embodiments, R⁹ can be an unsubstituted or a substituted 3- to 12-membered monocyclic heterocyclyl. In other embodiments, R⁹ can be an unsubstituted or a substituted 5- to 8-membered bicyclic heterocyclyl. In some embodiments, R⁹ can be an unsubstituted aryl(alkyl). In other embodiments, R⁹ can be a substituted aryl(alkyl). For example, R⁹ can be an unsubstituted or a substituted benzyl. In some embodiments, R⁹ can be an unsubstituted heteroaryl(alkyl). In other embodiments, R⁹ can be a substituted heteroaryl(alkyl). In some embodiments, R⁹ can be an unsubstituted heterocyclyl(alkyl). In other embodiments, R⁹ can be a substituted heterocyclyl(alkyl). The aryl, heteroaryl and heterocyclyl, including that those of an aryl(alkyl), heteroaryl(alkyl) and heterocyclyl(alkyl)) can be monocyclic or bicyclic (unless stated otherwise), and include 1, 2, 3, 4 or 5 heteroatoms independently selected from O (oxygen), S (sulfur) and N (nitrogen). Exemplary heteroaryls for R⁹ include, but are not limited to, furan, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine, pyrazine, indole, benzo[d]imidazole, pyrrolo[2,3-b]pyridine, pyrrolo[3,2-c]pyridine, pyrrolo[3,2-b]pyridine, thieno[2,3-b]pyrrole, benzofuran, benzo[b]thiophene, benzo[d]oxazole and benzo[d]thiazole. Examples of heterocyclyls for R⁹ include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, pyridin-2(1H)-one, pyridazin-3(2H)-one, morpholine, thiomorpholine, isoquinolin-1(2H)-one and 5-azaspiro[2.4]heptane. When R⁹ is aryl(alkyl), heteroaryl(alkyl) or heterocyclyl(alkyl), the alkyl linker can be 1, 2 or 3 alkylene groups, such as —CH₂—, —CH₂CH₂—, —CH₂CH₂CH₂—, —CH(CH₃)—, —C(CH₃)₃— and —CH₂—(CH₃)₃—. The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) of the alkyl linker include, but are not limited to, halogen, hydroxy and cyclopropyl (for example, —CH-(cyclopropyl)-), or two hydrogen on the same carbon can be replaced with a spiro-connected monocyclic C₃₋₄ cycloalkyl (for example,

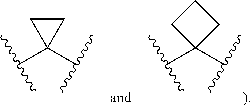 [SMILES: *C1(*)CC1 ; *C1(*)CCC1]

As provided herein, R⁹ can be substituted. For example, R⁹ can be substituted 1, 2, 3 or 4 times with a substituent independently selected from halogen, hydroxy, an unsubstituted C₁₋₄ alkyl, an unsubstituted C₁₋₄ alkoxy (for example —O(C₁₋₄ alkyl), such as methoxy, ethoxy, n-propoxy, iso-propoxy, n-butoxy, iso-butoxy, sec-butoxy and tert-butoxy), an unsubstituted C₁₋₄ haloalkyl, —C(═O)(an unsubstituted C₁₋₄ alkyl), —C(═O)(an unsubstituted C₁₋₄ haloalkyl), an unsubstituted monocyclic C₃₋₆ cycloalkyl, an unsubstituted phenyl, a substituted phenyl, an unsubstituted 5- or 6-membered heteroaryl, a substituted 5- or 6-membered heteroaryl, an unsubstituted 5- or 6-membered heterocyclyl and a substituted 5- or 6-membered heterocyclyl (for example a substituted phenyl, as substituted 5- or 6-membered heteroaryl and/or a substituted 5- or 6-membered heterocyclyl, which can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (for example —O(C₁₋₄ alkyl) such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CHF₂, —C(CH₃)F₂, —CH₂F, —CH(CH₃)F, —CH₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂ (an unsubstituted C₁₋₄ alkyl). The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) include, but are not limited to, halogen and hydroxy. The 5- or 6-membered heteroaryl and 5- or 6-membered heterocyclyl can include 1, 2 or 3 heteroatoms selected from O (oxygen), S (sulfur) and N (nitrogen).

In some embodiments, R⁹ can be an unsubstituted C-carboxy. In other embodiments, R⁹ can be a substituted C-carboxy. In still other embodiments, R⁹ can be an alkoxy. For example, in some embodiments, R⁹ can be —OR¹⁰, wherein R¹⁰ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl). In still other embodiments, R⁹ can be amino, mono-substituted amine or a di-substituted amine. In some embodiments, the amine can be —NR¹¹R¹², wherein R¹¹ and R¹² can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl). In yet still other embodiments, R⁹ can be C-amido. In some embodiments, R⁹ can be —C(═O)—NR^{13A}R^{13B} wherein R^{13A} can be hydrogen or an unsubstituted C₁₋₃ alkyl; and R^{13B} can be an unsubstituted or a substituted aryl, an unsubstituted or a substituted heteroaryl or an unsubstituted or a substituted 3- to 8-membered monocyclic heterocyclyl. In some embodiments, R^{13A} can be hydrogen; and R^{13B} can be an unsubstituted or a substituted phenyl, an unsubstituted or a substituted monocyclic 5- or 6-membered heteroaryl or an unsubstituted or a substituted 3- to 8-membered monocyclic heterocyclyl.

Various examples of R⁹ groups include the following:

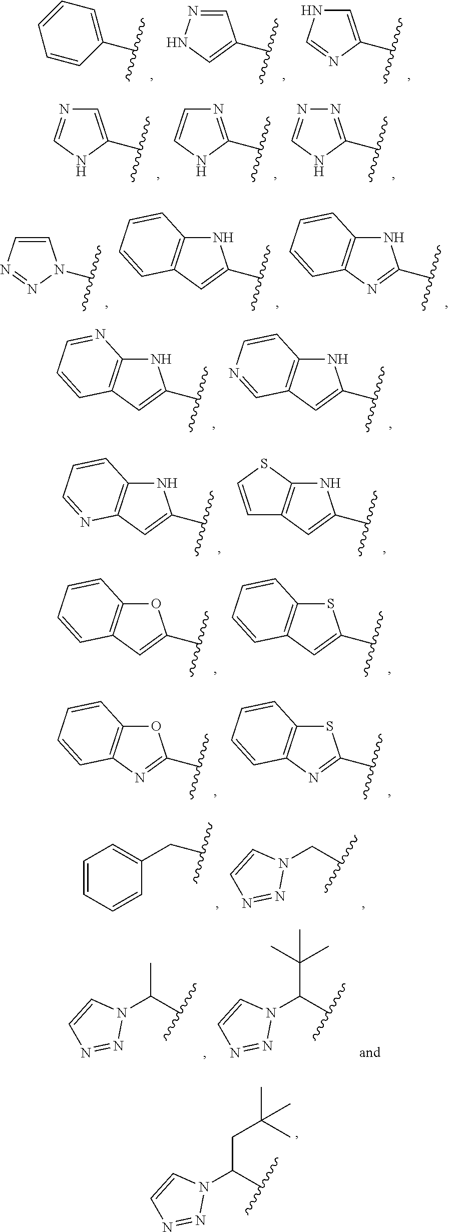

wherein each is unsubstituted or substituted (including the nitrogen). Other examples of R⁹ groups include the following:

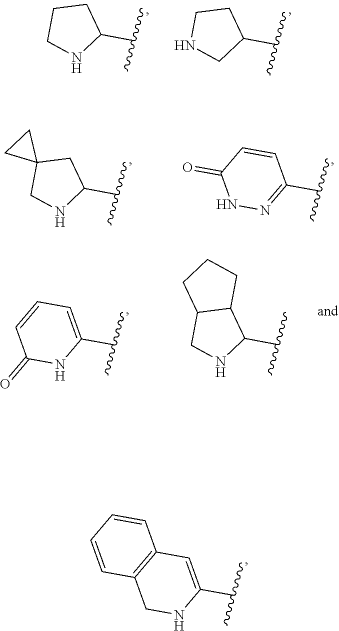

wherein each is unsubstituted or substituted (including the nitrogen). When R⁹ is substituted, R⁹ can be substituted with a variety of substituents. For example, R⁹ can be substituted 1, 2, 3, or more than 3 times with a substituent independently selected from halogen, (such as F and Cl), an unsubstituted C₁₋₄ alkyl, an unsubstituted C₁₋₄ haloalkyl, hydroxy, an unsubstituted C₁₋₄ alkoxy, —C(═O)(an unsubstituted C₁₋₄ alkyl), —C(═O)(an unsubstituted C₁₋₄ haloalkyl) and an unsubstituted or a substituted phenyl (for example, an unsubstituted phenyl or a phenyl substituted with 1, 2 or 3 substituents independently selected from halogen, an unsubstituted C₁₋₄ alkyl, an unsubstituted C₁₋₄ haloalkyl and an unsubstituted C₁₋₄ alkoxy).

Examples of substituted R⁹ groups include the following:

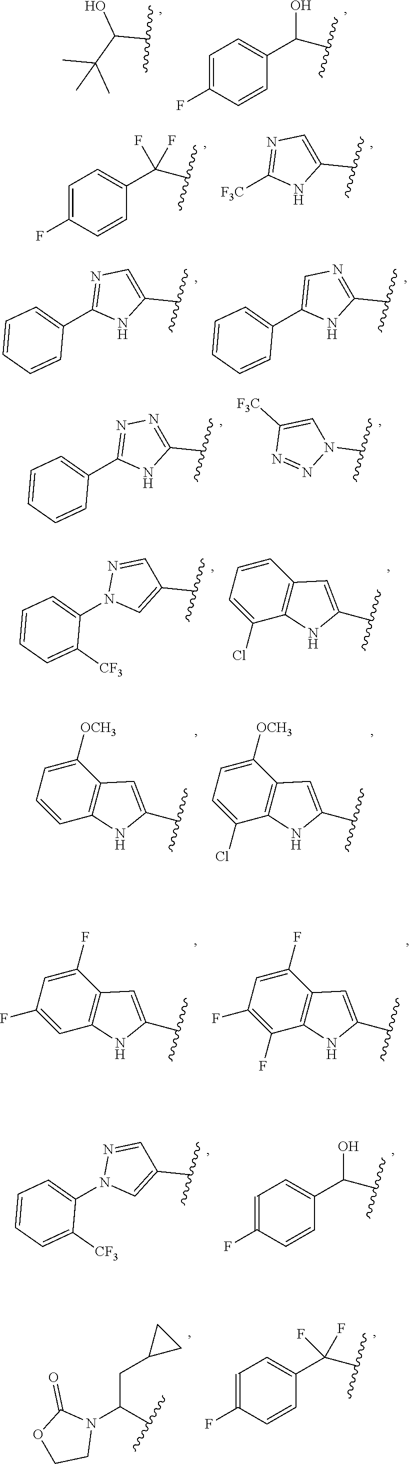

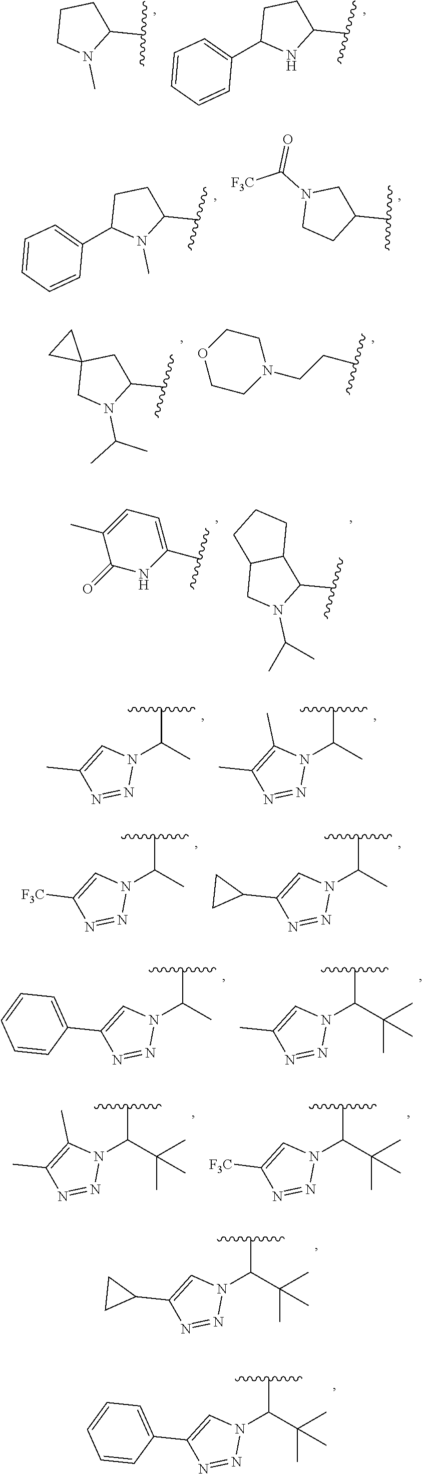

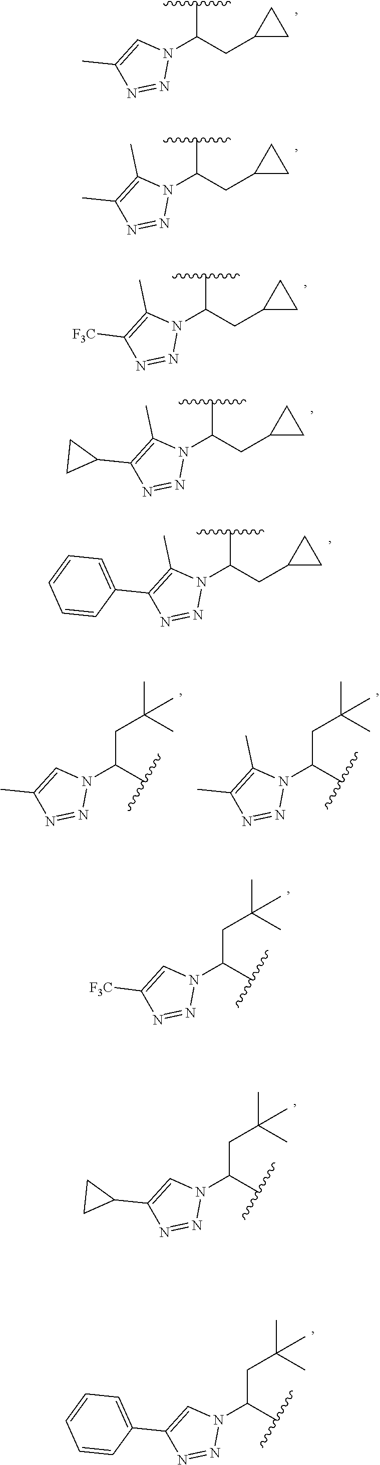

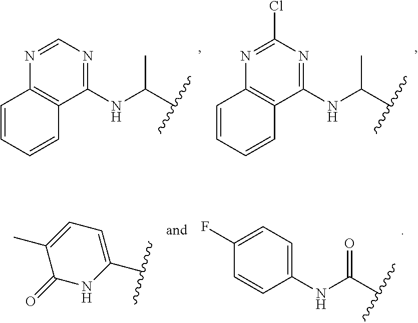

Further examples of R⁹ include the following:

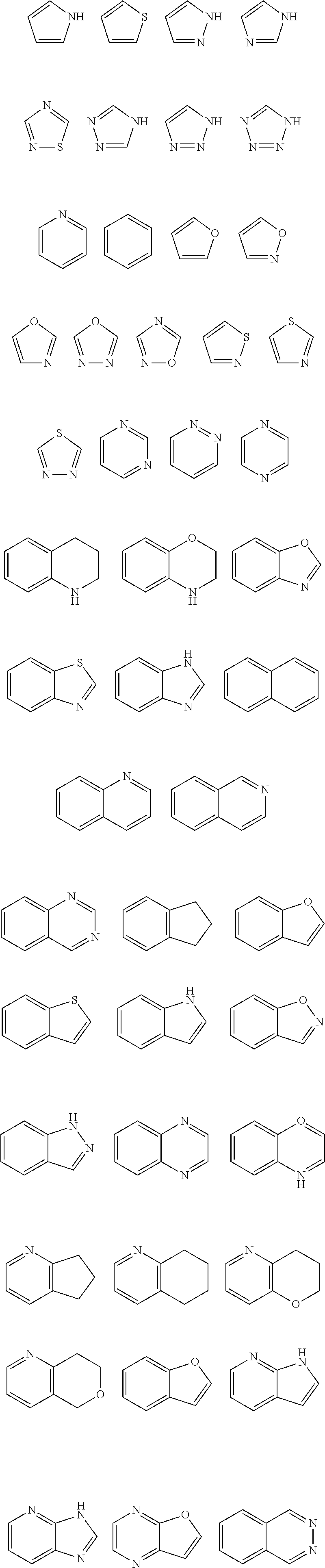

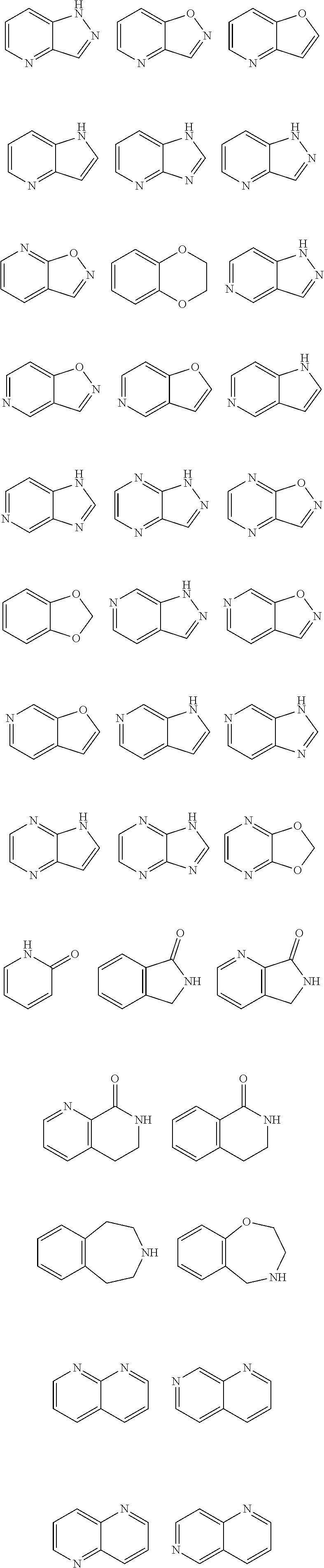

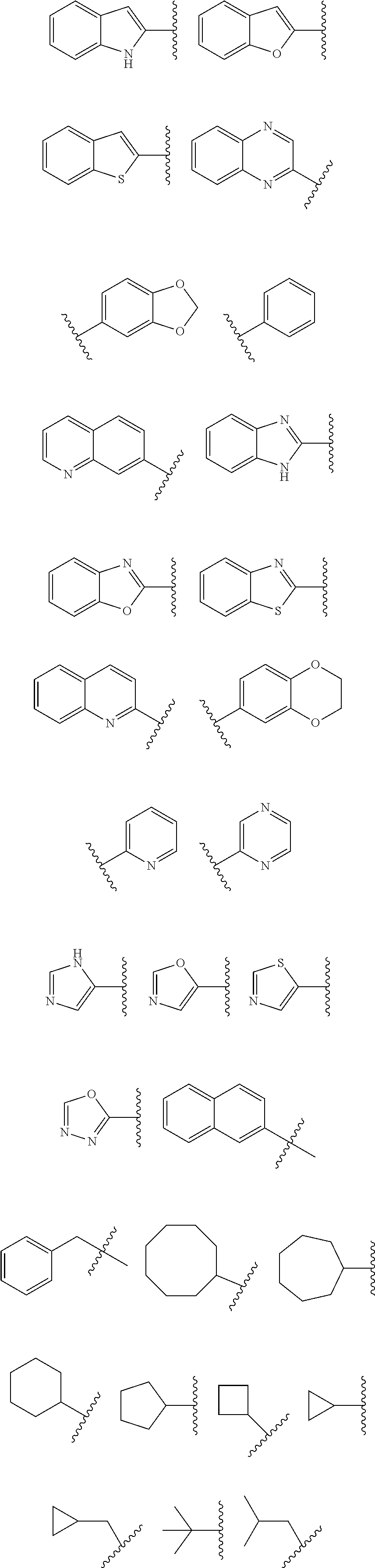

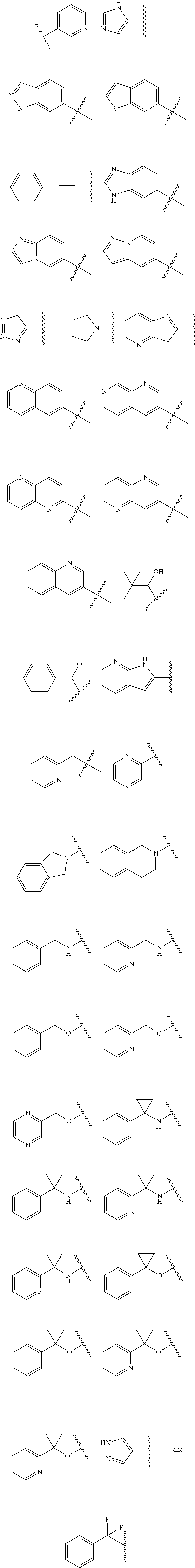

wherein each is unsubstituted or substituted.

Exemplary R³ groups include the following:

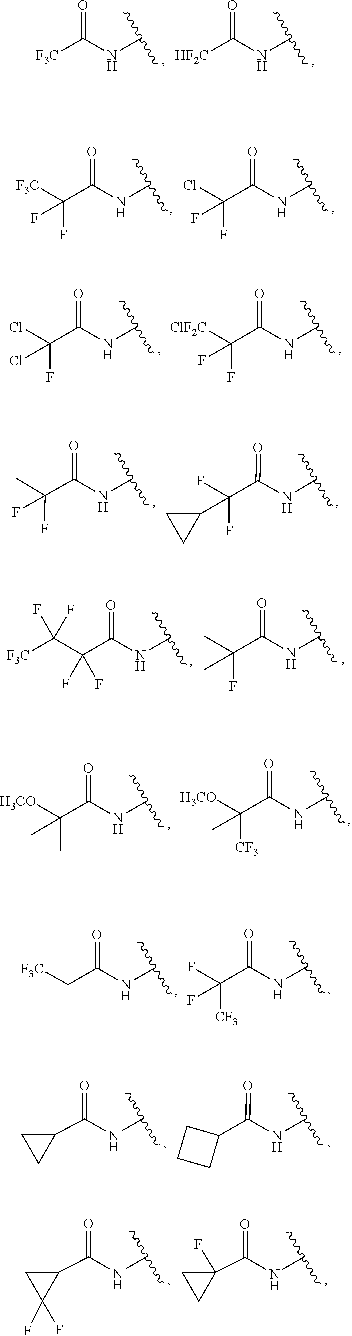

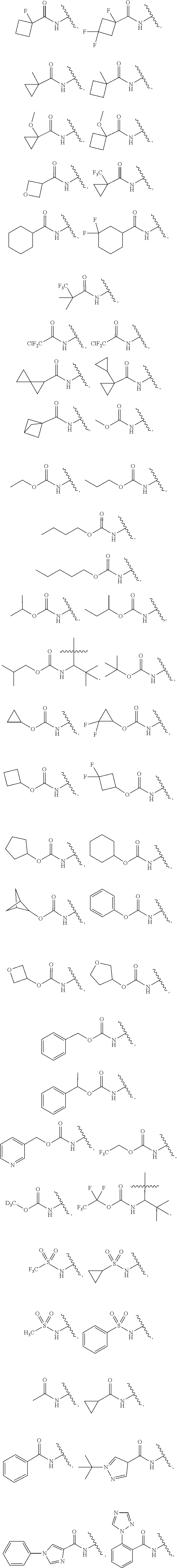

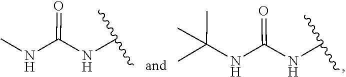

wherein each phenyl and can be substituted or unsubstituted as described herein. As examples, the phenyl can be substituted with 1, 2 or 3 substituents independently selected from halogen for example, fluoro and/or chloro, an unsubstituted C₁₋₄ alkyl, an unsubstituted C₁₋₄ haloalkyl and an unsubstituted C₁₋₄ alkoxy. Examples of substituted phenyls within R⁸ include

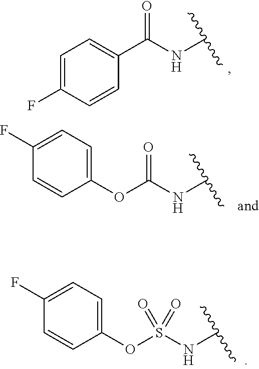

Further examples of R³ groups include:

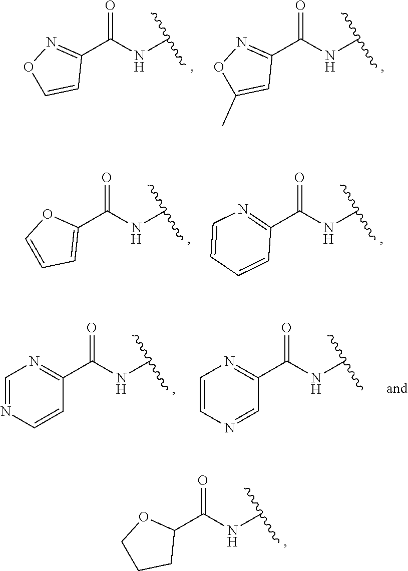

wherein each moiety is unsubstituted or substituted.

Examples of compounds of Formula (I), include the following:

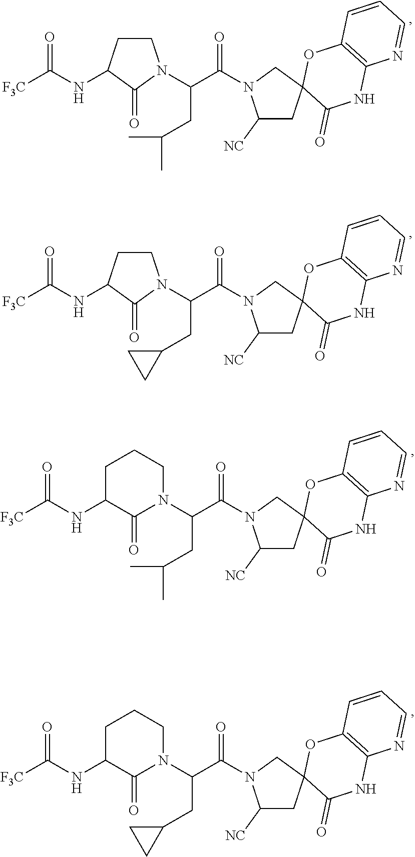 [SMILES: CC(C)CC(C(=O)N1CC2(CC1C#N)Oc1cccnc1NC2=O)N1CCC(NC(=O)C(F)(F)F)C1=O ; N#CC1CC2(CN1C(=O)C(CC1CC1)N1CCC(NC(=O)C(F)(F)F)C1=O)Oc1cccnc1NC2=O ; CC(C)CC(C(=O)N1CC2(CC1C#N)Oc1cccnc1NC2=O)N1CCCC(NC(=O)C(F)(F)F)C1=O ; N#CC1CC2(CN1C(=O)C(CC1CC1)N1CCCC(NC(=O)C(F)(F)F)C1=O)Oc1cccnc1NC2=O]

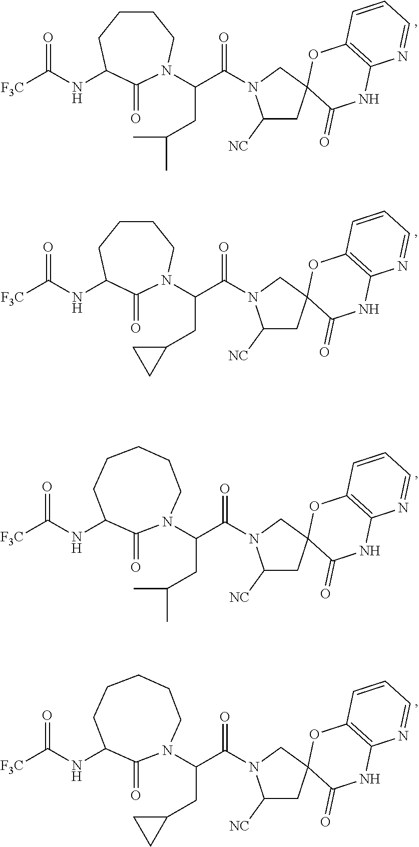 [SMILES: CC(C)CC(C(=O)N1CC2(CC1C#N)Oc1cccnc1NC2=O)N1CCCCC(NC(=O)C(F)(F)F)C1=O ; N#CC1CC2(CN1C(=O)C(CC1CC1)N1CCCCC(NC(=O)C(F)(F)F)C1=O)Oc1cccnc1NC2=O ; CC(C)CC(C(=O)N1CC2(CC1C#N)Oc1cccnc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1cccnc1NC2=O]

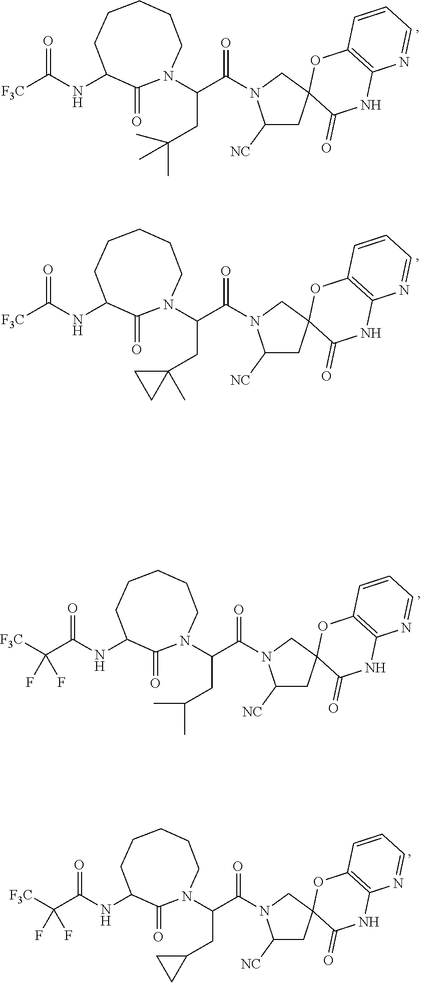 [SMILES: CC(C)(C)CC(C(=O)N1CC2(CC1C#N)Oc1cccnc1NC2=O)N1CCCCCC(NC(=O)C(F)(F)F)C1=O ; CC1(CC(C(=O)N2CC3(CC2C#N)Oc2cccnc2NC3=O)N2CCCCCC(NC(=O)C(F)(F)F)C2=O)CC1 ; CC(C)CC(C(=O)N1CC2(CC1C#N)Oc1cccnc1NC2=O)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)Oc1cccnc1NC2=O]

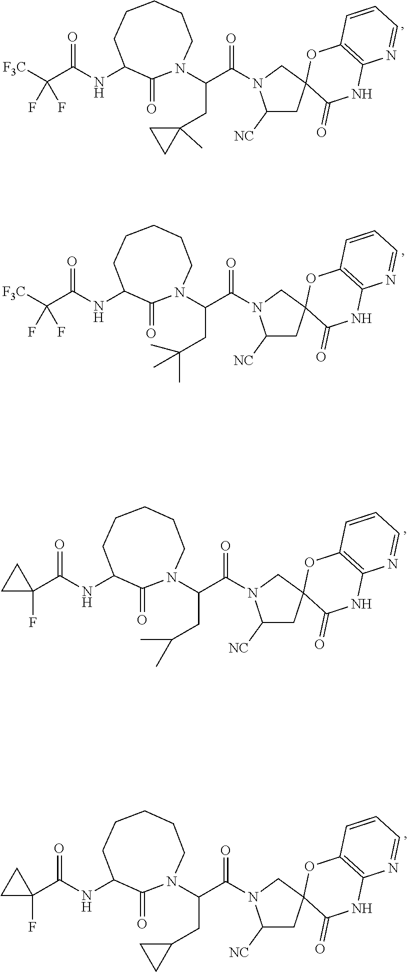 [SMILES: CC1(CC(C(=O)N2CC3(CC2C#N)Oc2cccnc2NC3=O)N2CCCCCC(NC(=O)C(F)(F)C(F)(F)F)C2=O)CC1 ; CC(C)(C)CC(C(=O)N1CC2(CC1C#N)Oc1cccnc1NC2=O)N1CCCCCC(NC(=O)C(F)(F)C(F)(F)F)C1=O ; CC(C)CC(C(=O)N1CC2(CC1C#N)Oc1cccnc1NC2=O)N1CCCCCC(NC(=O)C2(F)CC2)C1=O ; N#CC1CC2(CN1C(=O)C(CC1CC1)N1CCCCCC(NC(=O)C3(F)CC3)C1=O)Oc1cccnc1NC2=O]

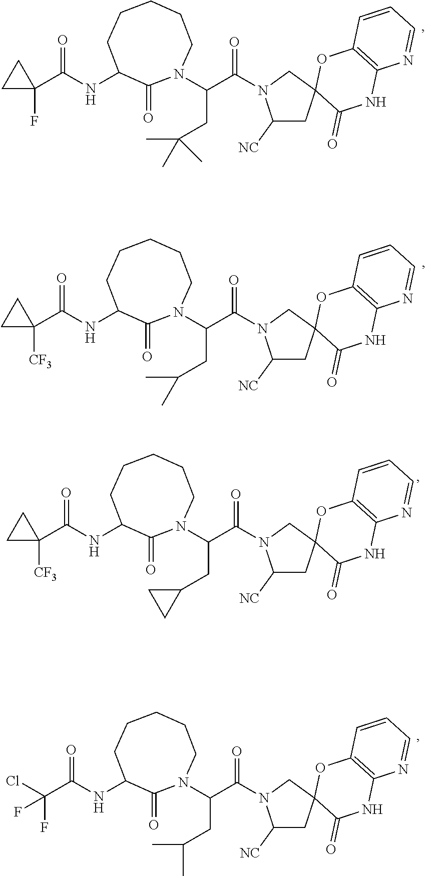 [SMILES: CC(C)(C)CC(C(=O)N1CC2(CC1C#N)Oc1cccnc1NC2=O)N1CCCCCC(NC(=O)C2(F)CC2)C1=O ; CC(C)CC(C(=O)N1CC2(CC1C#N)Oc1cccnc1NC2=O)N1CCCCCC(NC(=O)C2(C(F)(F)F)CC2)C1=O ; N#CC1CC2(CN1C(=O)C(CC1CC1)N1CCCCCC(NC(=O)C3(C(F)(F)F)CC3)C1=O)Oc1cccnc1NC2=O ; CC(C)CC(C(=O)N1CC2(CC1C#N)Oc1cccnc1NC2=O)N1CCCCCC(NC(=O)C(F)(F)Cl)C1=O]

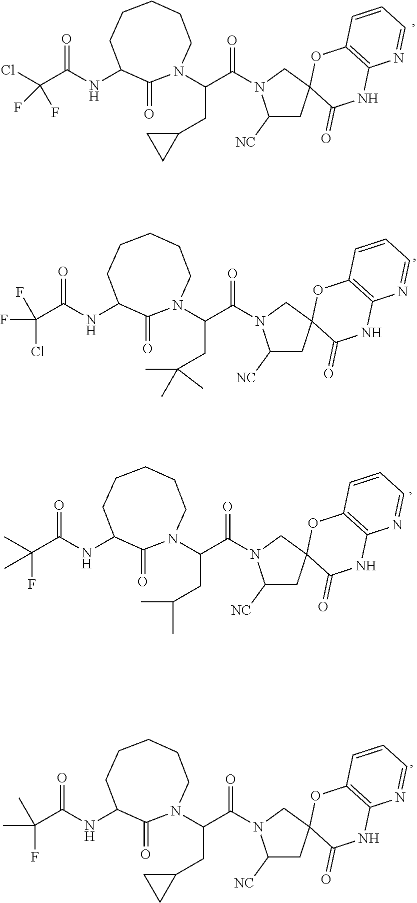 [SMILES: N#CC1CC2(CN1C(=O)C(CC1CC1)N1CCCCCC(NC(=O)C(F)(F)Cl)C1=O)Oc1cccnc1NC2=O ; CC(C)(C)CC(C(=O)N1CC2(CC1C#N)Oc1cccnc1NC2=O)N1CCCCCC(NC(=O)C(F)(F)Cl)C1=O ; CC(C)CC(C(=O)N1CC2(CC1C#N)Oc1cccnc1NC2=O)N1CCCCCC(NC(=O)C(C)(C)F)C1=O ; CC(C)(F)C(=O)NC1CCCCCN(C(CC2CC2)C(=O)N2CC3(CC2C#N)Oc2cccnc2NC3=O)C1=O]

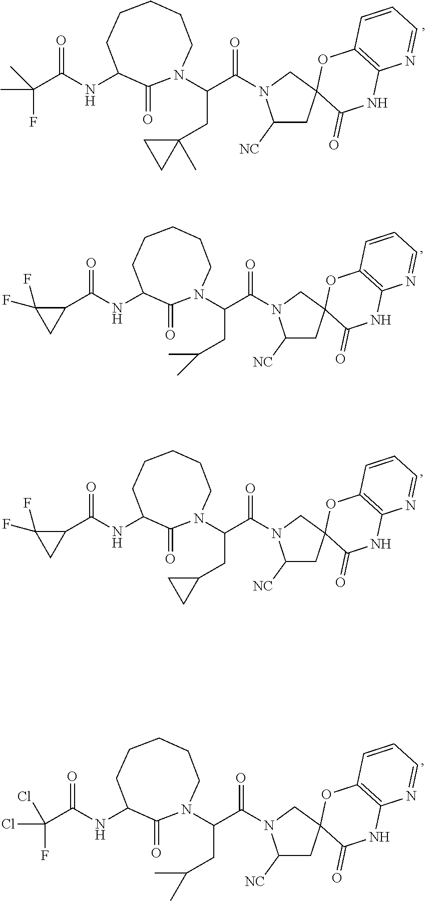 [SMILES: CC1(CC(C(=O)N2CC3(CC2C#N)Oc2cccnc2NC3=O)N2CCCCCC(NC(=O)C(C)(C)F)C2=O)CC1 ; CC(C)CC(C(=O)N1CC2(CC1C#N)Oc1cccnc1NC2=O)N1CCCCCC(NC(=O)C2CC2(F)F)C1=O ; N#CC1CC2(CN1C(=O)C(CC1CC1)N1CCCCCC(NC(=O)C3CC3(F)F)C1=O)Oc1cccnc1NC2=O ; CC(C)CC(C(=O)N1CC2(CC1C#N)Oc1cccnc1NC2=O)N1CCCCCC(NC(=O)C(F)(Cl)Cl)C1=O]

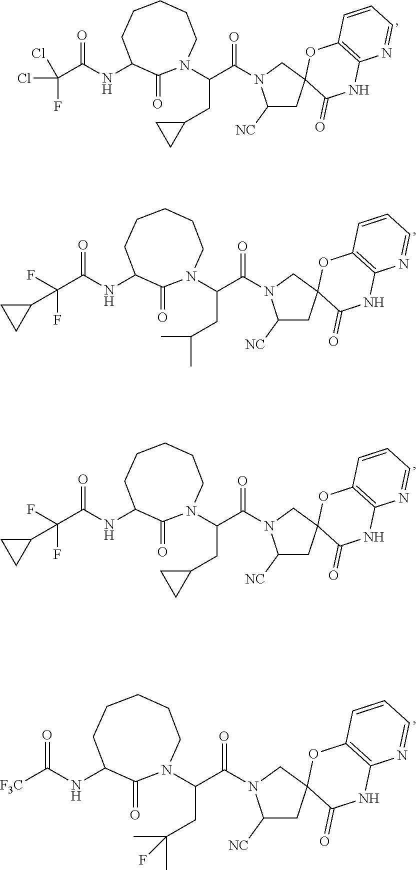 [SMILES: N#CC1CC2(CN1C(=O)C(CC1CC1)N1CCCCCC(NC(=O)C(F)(Cl)Cl)C1=O)Oc1cccnc1NC2=O ; CC(C)CC(C(=O)N1CC2(CC1C#N)Oc1cccnc1NC2=O)N1CCCCCC(NC(=O)C(F)(F)C2CC2)C1=O ; N#CC1CC2(CN1C(=O)C(CC1CC1)N1CCCCCC(NC(=O)C(F)(F)C3CC3)C1=O)Oc1cccnc1NC2=O ; CC(C)(F)CC(C(=O)N1CC2(CC1C#N)Oc1cccnc1NC2=O)N1CCCCCC(NC(=O)C(F)(F)F)C1=O]

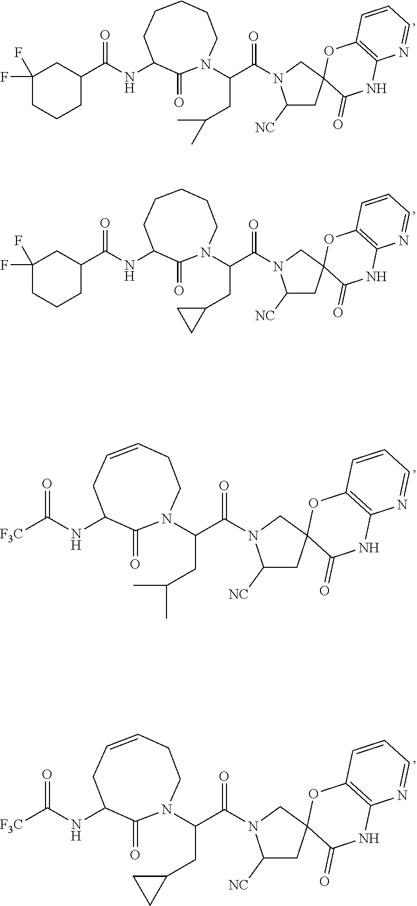 [SMILES: CC(C)CC(C(=O)N1CC2(CC1C#N)Oc1cccnc1NC2=O)N1CCCCCC(NC(=O)C2CCCC(F)(F)C2)C1=O ; N#CC1CC2(CN1C(=O)C(CC1CC1)N1CCCCCC(NC(=O)C3CCCC(F)(F)C3)C1=O)Oc1cccnc1NC2=O ; CC(C)CC(C(=O)N1CC2(CC1C#N)Oc1cccnc1NC2=O)N1CC/C=C\CC(NC(=O)C(F)(F)F)C1=O ; N#CC1CC2(CN1C(=O)C(CC1CC1)N1CC/C=C\CC(NC(=O)C(F)(F)F)C1=O)Oc1cccnc1NC2=O]

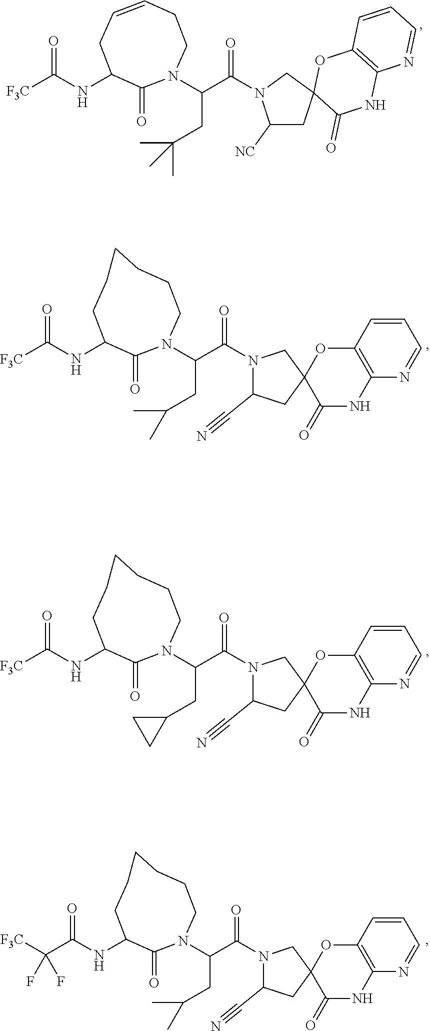 [SMILES: CC(C)(C)CC(C(=O)N1CC2(CC1C#N)Oc1cccnc1NC2=O)N1CC/C=C\CC(NC(=O)C(F)(F)F)C1=O ; CC(C)CC(C(=O)N1CC2(CC1C#N)Oc1cccnc1NC2=O)N1CCCCCCC(NC(=O)C(F)(F)F)C1=O ; N#CC1CC2(CN1C(=O)C(CC1CC1)N1CCCCCCC(NC(=O)C(F)(F)F)C1=O)Oc1cccnc1NC2=O ; CC(C)CC(C(=O)N1CC2(CC1C#N)Oc1cccnc1NC2=O)N1CCCCCCC(NC(=O)C(F)(F)C(F)(F)F)C1=O]

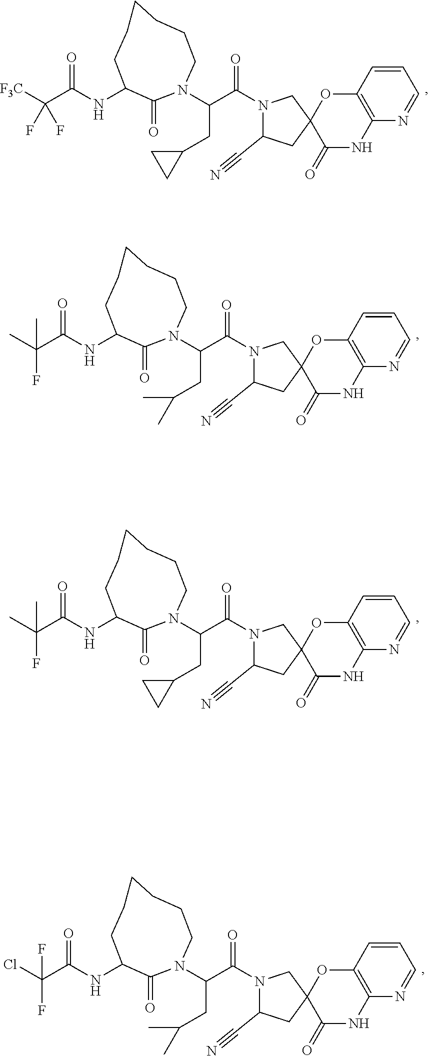 [SMILES: N#CC1CC2(CN1C(=O)C(CC1CC1)N1CCCCCCC(NC(=O)C(F)(F)C(F)(F)F)C1=O)Oc1cccnc1NC2=O ; CC(C)CC(C(=O)N1CC2(CC1C#N)Oc1cccnc1NC2=O)N1CCCCCCC(NC(=O)C(C)(C)F)C1=O ; CC(C)(F)C(=O)NC1CCCCCCN(C(CC2CC2)C(=O)N2CC3(CC2C#N)Oc2cccnc2NC3=O)C1=O ; CC(C)CC(C(=O)N1CC2(CC1C#N)Oc1cccnc1NC2=O)N1CCCCCCC(NC(=O)C(F)(F)Cl)C1=O]

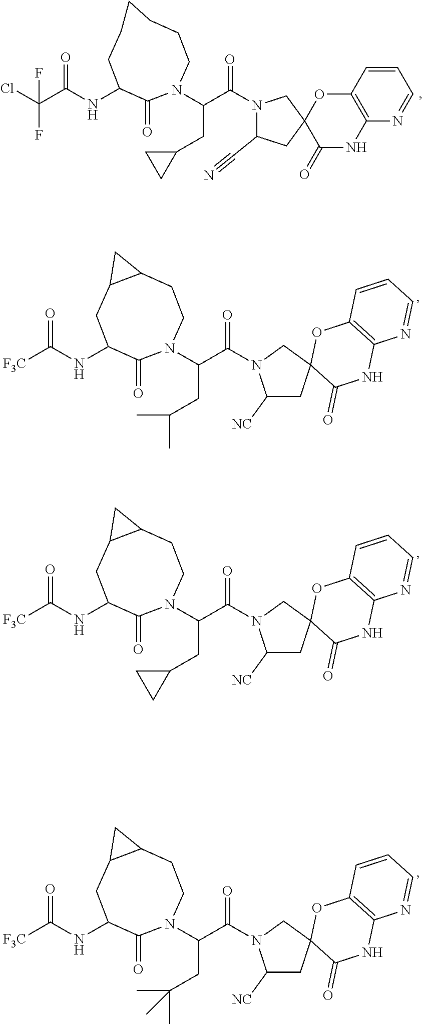 [SMILES: N#CC1CC2(CN1C(=O)C(CC1CC1)N1CCCCCCC(NC(=O)C(F)(F)Cl)C1=O)Oc1cccnc1NC2=O ; CC(C)CC(C(=O)N1CC2(CC1C#N)Oc1cccnc1NC2=O)N1CCC2CC2CC(NC(=O)C(F)(F)F)C1=O ; N#CC1CC2(CN1C(=O)C(CC1CC1)N1CCC3CC3CC(NC(=O)C(F)(F)F)C1=O)Oc1cccnc1NC2=O ; CC(C)(C)CC(C(=O)N1CC2(CC1C#N)Oc1cccnc1NC2=O)N1CCC2CC2CC(NC(=O)C(F)(F)F)C1=O]

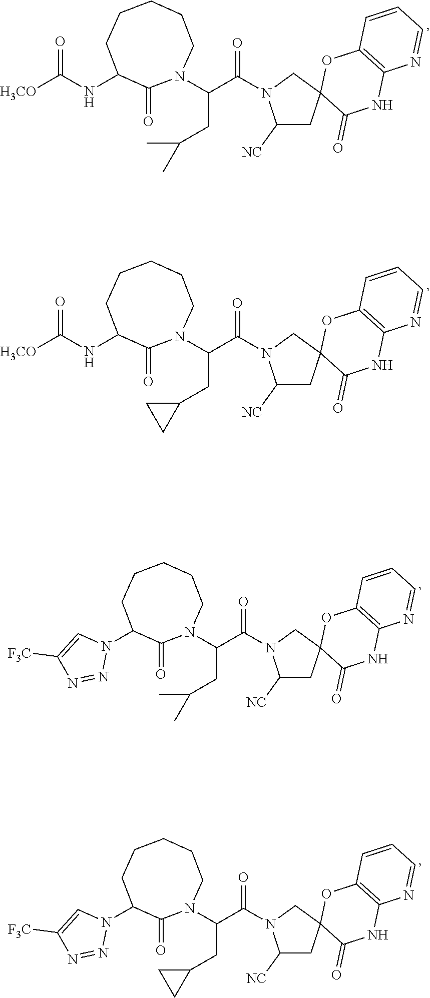 [SMILES: COC(=O)NC1CCCCCN(C(CC(C)C)C(=O)N2CC3(CC2C#N)Oc2cccnc2NC3=O)C1=O ; COC(=O)NC1CCCCCN(C(CC2CC2)C(=O)N2CC3(CC2C#N)Oc2cccnc2NC3=O)C1=O ; CC(C)CC(C(=O)N1CC2(CC1C#N)Oc1cccnc1NC2=O)N1CCCCCC(n2cc(C(F)(F)F)nn2)C1=O ; N#CC1CC2(CN1C(=O)C(CC1CC1)N1CCCCCC(n3cc(C(F)(F)F)nn3)C1=O)Oc1cccnc1NC2=O]

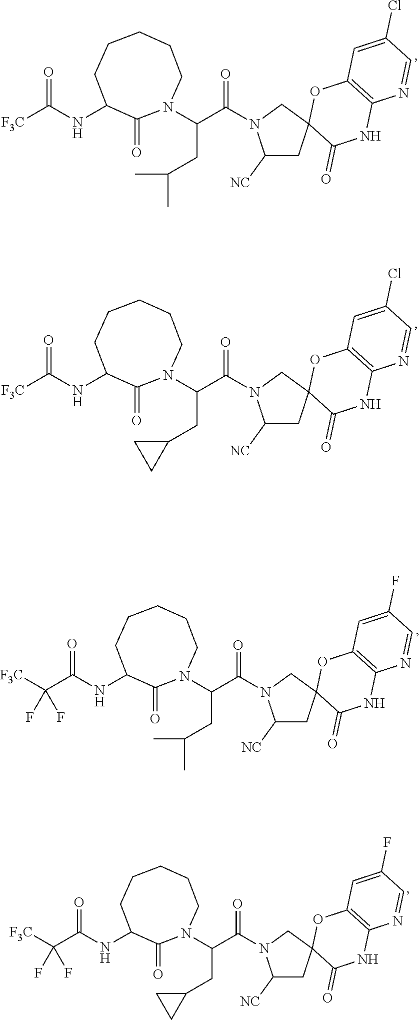 [SMILES: CC(C)CC(C(=O)N1CC2(CC1C#N)Oc1cc(Cl)cnc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1cc(Cl)cnc1NC2=O ; CC(C)CC(C(=O)N1CC2(CC1C#N)Oc1cc(F)cnc1NC2=O)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)Oc1cc(F)cnc1NC2=O]

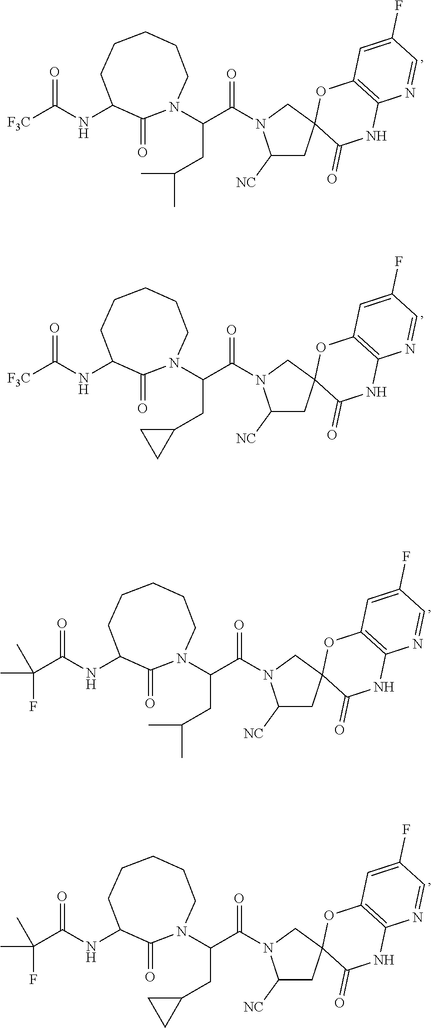 [SMILES: CC(C)CC(C(=O)N1CC2(CC1C#N)Oc1cc(F)cnc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1cc(F)cnc1NC2=O ; CC(C)CC(C(=O)N1CC2(CC1C#N)Oc1cc(F)cnc1NC2=O)N1CCCCCC(NC(=O)C(C)(C)F)C1=O ; CC(C)(F)C(=O)NC1CCCCCN(C(CC2CC2)C(=O)N2CC3(CC2C#N)Oc2cc(F)cnc2NC3=O)C1=O]

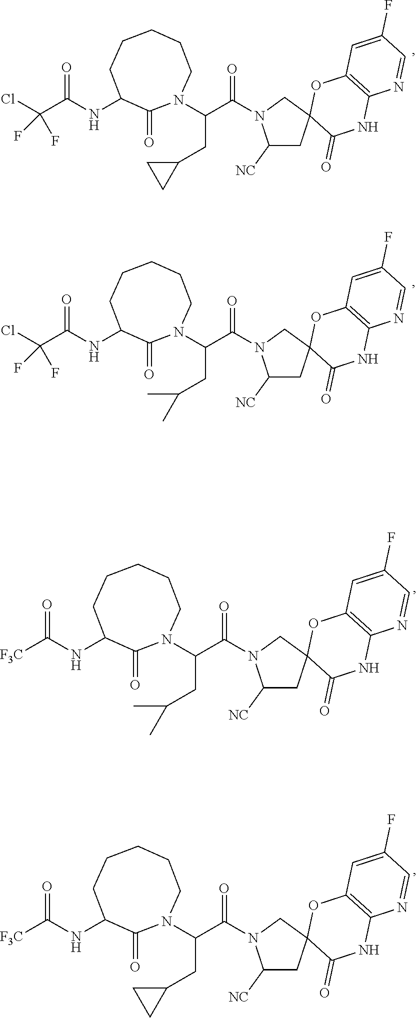 [SMILES: N#CC1CC2(CN1C(=O)C(CC1CC1)N1CCCCCC(NC(=O)C(F)(F)Cl)C1=O)Oc1cc(F)cnc1NC2=O ; CC(C)CC(C(=O)N1CC2(CC1C#N)Oc1cc(F)cnc1NC2=O)N1CCCCCC(NC(=O)C(F)(F)Cl)C1=O ; CC(C)CC(C(=O)N1CC2(CC1C#N)Oc1cc(F)cnc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1cc(F)cnc1NC2=O]

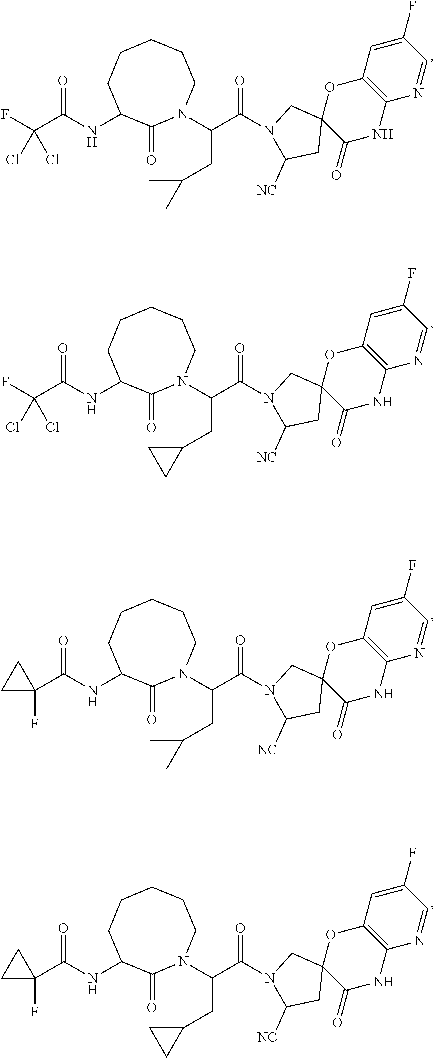 [SMILES: CC(C)CC(C(=O)N1CC2(CC1C#N)Oc1cc(F)cnc1NC2=O)N1CCCCCC(NC(=O)C(F)(Cl)Cl)C1=O ; N#CC1CC2(CN1C(=O)C(CC1CC1)N1CCCCCC(NC(=O)C(F)(Cl)Cl)C1=O)Oc1cc(F)cnc1NC2=O ; CC(C)CC(C(=O)N1CC2(CC1C#N)Oc1cc(F)cnc1NC2=O)N1CCCCCC(NC(=O)C2(F)CC2)C1=O ; N#CC1CC2(CN1C(=O)C(CC1CC1)N1CCCCCC(NC(=O)C3(F)CC3)C1=O)Oc1cc(F)cnc1NC2=O]

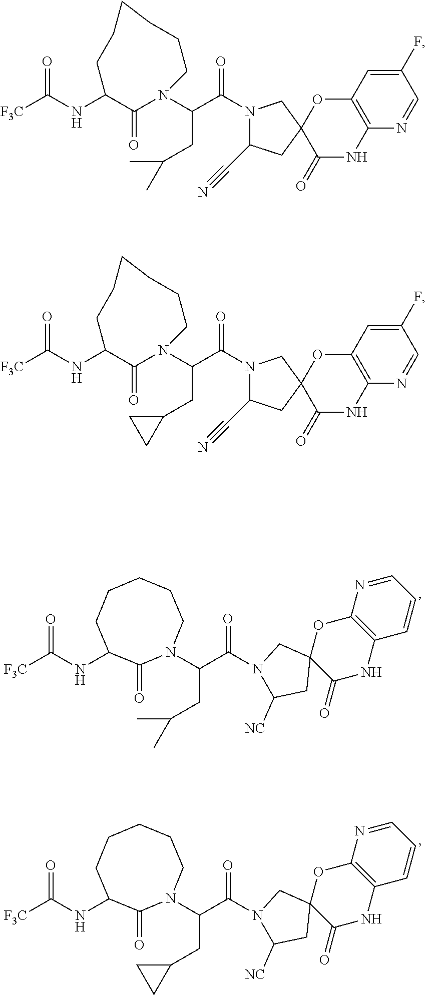 [SMILES: CC(C)CC(C(=O)N1CC2(CC1C#N)Oc1cc(F)cnc1NC2=O)N1CCCCCCC(NC(=O)C(F)(F)F)C1=O ; N#CC1CC2(CN1C(=O)C(CC1CC1)N1CCCCCCC(NC(=O)C(F)(F)F)C1=O)Oc1cc(F)cnc1NC2=O ; CC(C)CC(C(=O)N1CC2(CC1C#N)Oc1ncccc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1ncccc1NC2=O]

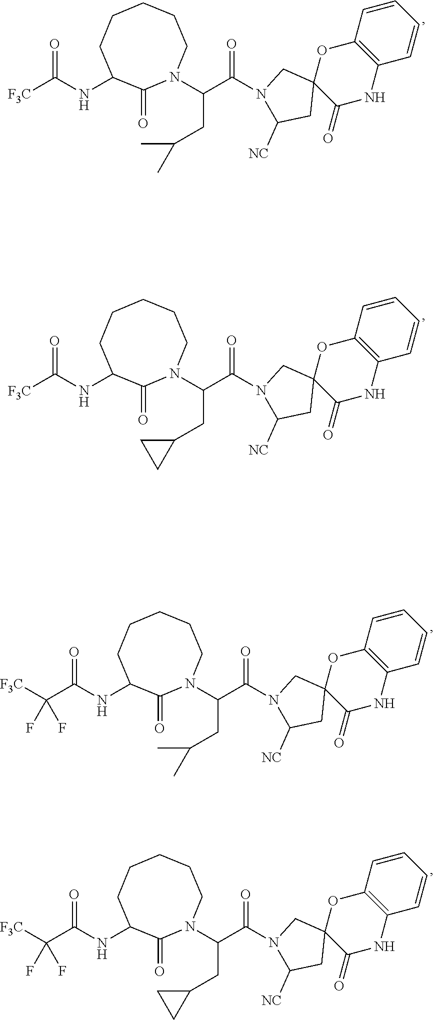 [SMILES: CC(C)CC(C(=O)N1CC2(CC1C#N)Oc1ccccc1NC2=O)N1CCCCCC(NC(=O)C(F)(F)F)C1=O ; N#CC1CC2(CN1C(=O)C(CC1CC1)N1CCCCCC(NC(=O)C(F)(F)F)C1=O)Oc1ccccc1NC2=O ; CC(C)CC(C(=O)N1CC2(CC1C#N)Oc1ccccc1NC2=O)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)Oc1ccccc1NC2=O]

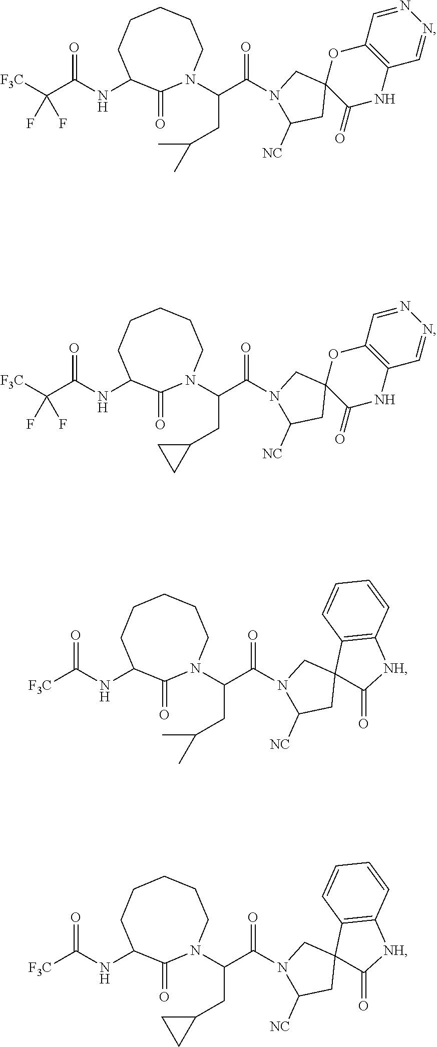 [SMILES: CC(C)CC(C(=O)N1CC2(CC1C#N)Oc1cnncc1NC2=O)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)Oc1cnncc1NC2=O ; CC(C)CC(C(=O)N1CC2(CC1C#N)C(=O)Nc1ccccc12)N1CCCCCC(NC(=O)C(F)(F)F)C1=O ; N#CC1CC2(CN1C(=O)C(CC1CC1)N1CCCCCC(NC(=O)C(F)(F)F)C1=O)C(=O)Nc1ccccc12]

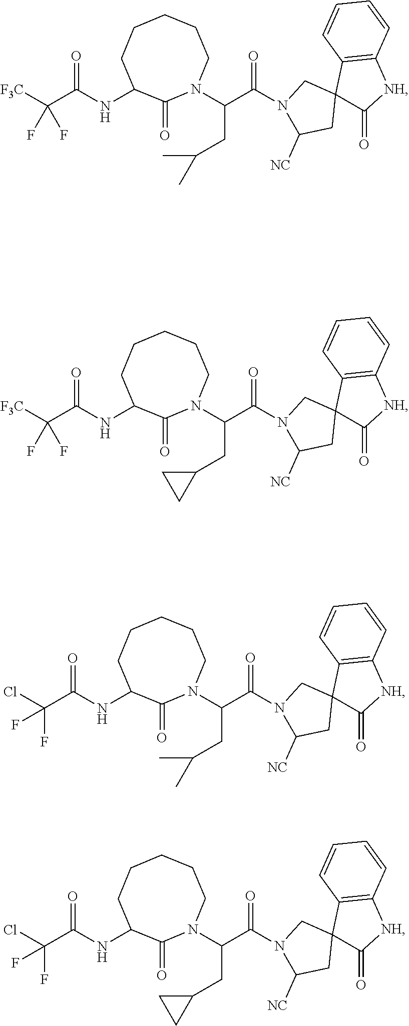 [SMILES: CC(C)CC(C(=O)N1CC2(CC1C#N)C(=O)Nc1ccccc12)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)C(=O)Nc1ccccc12 ; CC(C)CC(C(=O)N1CC2(CC1C#N)C(=O)Nc1ccccc12)N1CCCCCC(NC(=O)C(F)(F)Cl)C1=O ; N#CC1CC2(CN1C(=O)C(CC1CC1)N1CCCCCC(NC(=O)C(F)(F)Cl)C1=O)C(=O)Nc1ccccc12]

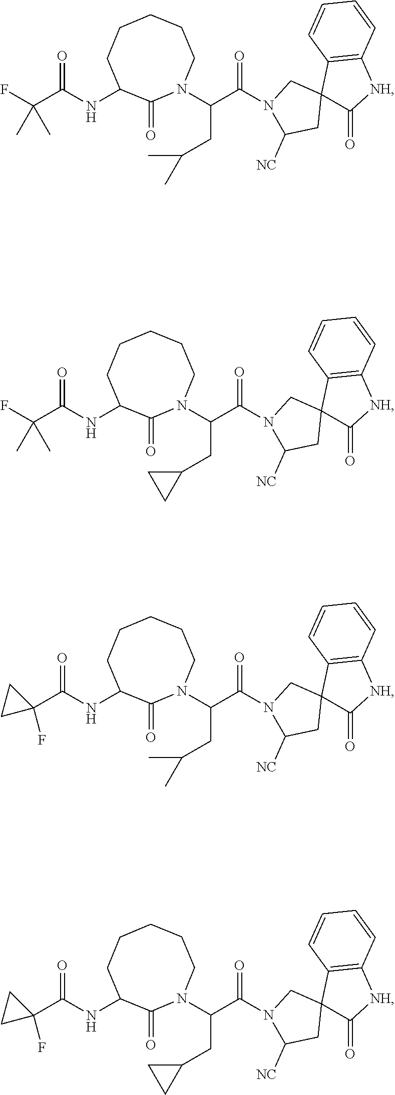 [SMILES: CC(C)CC(C(=O)N1CC2(CC1C#N)C(=O)Nc1ccccc12)N1CCCCCC(NC(=O)C(C)(C)F)C1=O ; CC(C)(F)C(=O)NC1CCCCCN(C(CC2CC2)C(=O)N2CC3(CC2C#N)C(=O)Nc2ccccc23)C1=O ; CC(C)CC(C(=O)N1CC2(CC1C#N)C(=O)Nc1ccccc12)N1CCCCCC(NC(=O)C2(F)CC2)C1=O ; N#CC1CC2(CN1C(=O)C(CC1CC1)N1CCCCCC(NC(=O)C3(F)CC3)C1=O)C(=O)Nc1ccccc12]

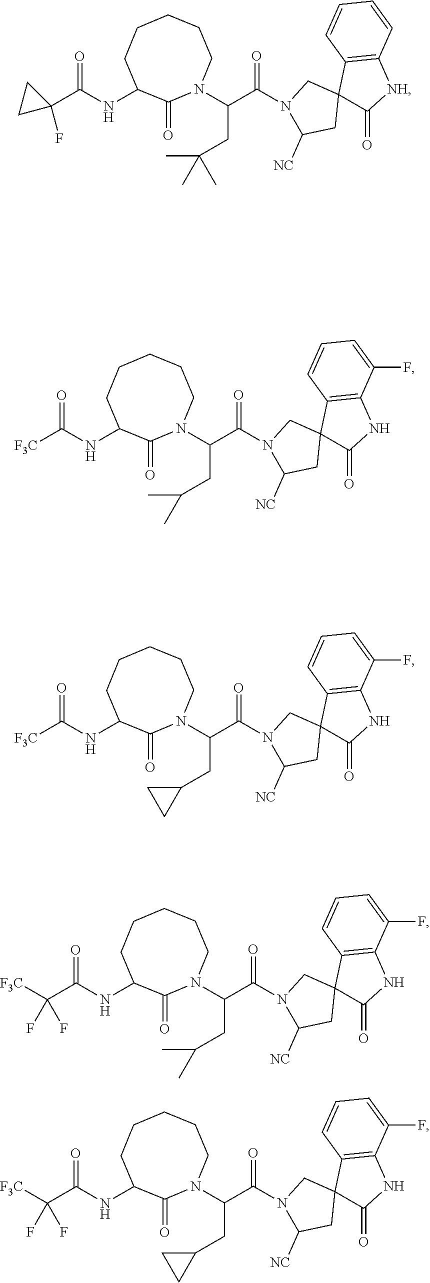

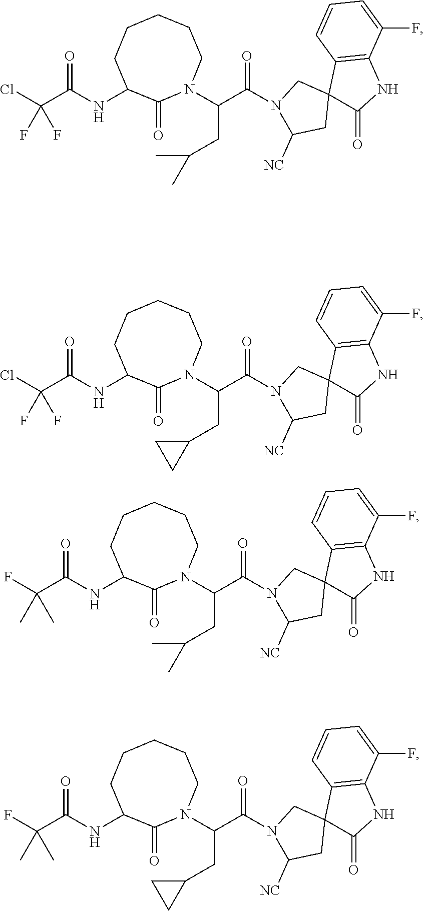

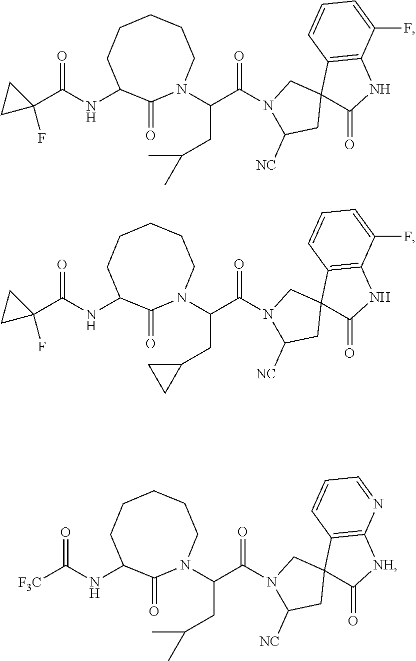

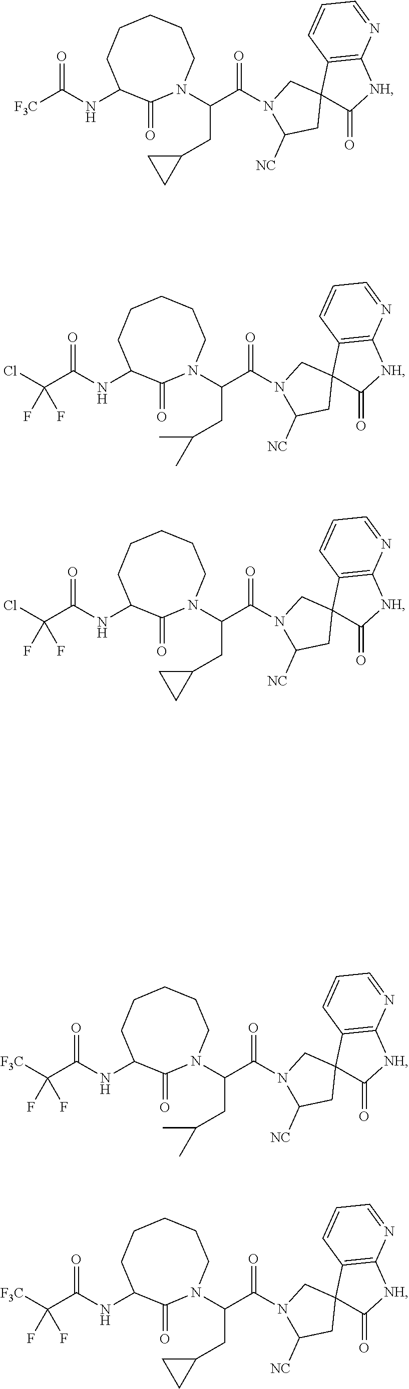 [SMILES: N#CC1CC2(CN1C(=O)C(CC1CC1)N1CCCCCC(NC(=O)C(F)(F)F)C1=O)C(=O)Nc1ncccc12 ; CC(C)CC(C(=O)N1CC2(CC1C#N)C(=O)Nc1ncccc12)N1CCCCCC(NC(=O)C(F)(F)Cl)C1=O ; N#CC1CC2(CN1C(=O)C(CC1CC1)N1CCCCCC(NC(=O)C(F)(F)Cl)C1=O)C(=O)Nc1ncccc12 ; CC(C)CC(C(=O)N1CC2(CC1C#N)C(=O)Nc1ncccc12)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)C(=O)Nc1ncccc12]

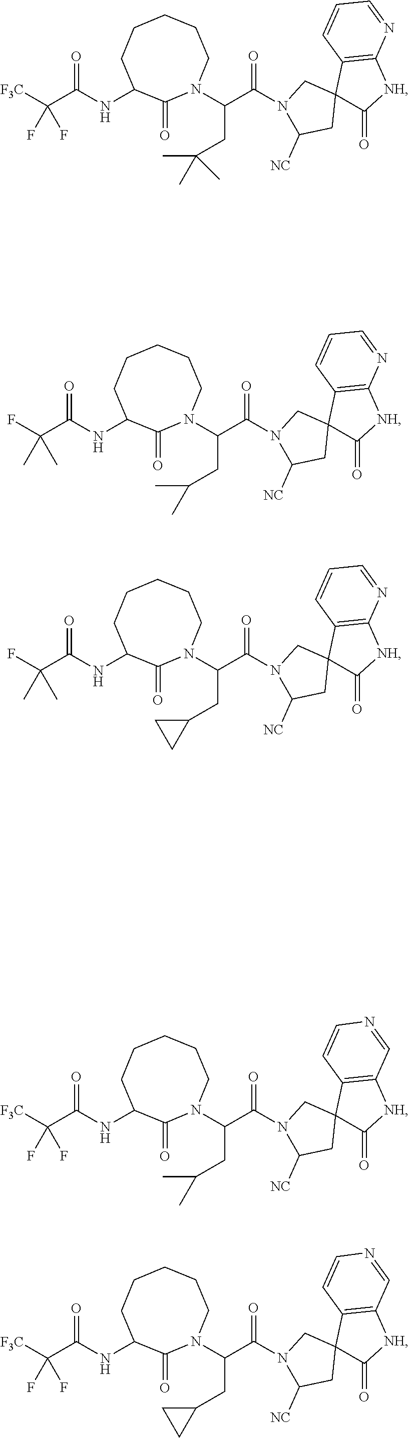 [SMILES: CC(C)(C)CC(C(=O)N1CC2(CC1C#N)C(=O)Nc1ncccc12)N1CCCCCC(NC(=O)C(F)(F)C(F)(F)F)C1=O ; CC(C)CC(C(=O)N1CC2(CC1C#N)C(=O)Nc1ncccc12)N1CCCCCC(NC(=O)C(C)(C)F)C1=O ; CC(C)(F)C(=O)NC1CCCCCN(C(CC2CC2)C(=O)N2CC3(CC2C#N)C(=O)Nc2ncccc23)C1=O ; CC(C)CC(C(=O)N1CC2(CC1C#N)C(=O)Nc1cnccc12)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)C(=O)Nc1cnccc12]

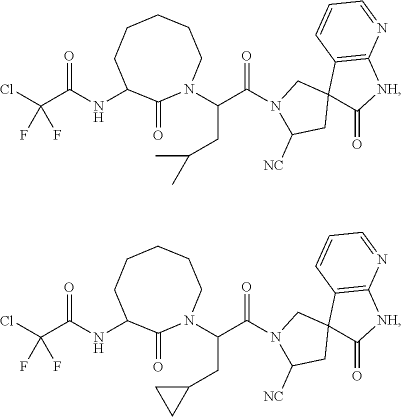 [SMILES: CC(C)CC(C(=O)N1CC2(CC1C#N)C(=O)Nc1ncccc12)N1CCCCCC(NC(=O)C(F)(F)Cl)C1=O ; N#CC1CC2(CN1C(=O)C(CC1CC1)N1CCCCCC(NC(=O)C(F)(F)Cl)C1=O)C(=O)Nc1ncccc12]

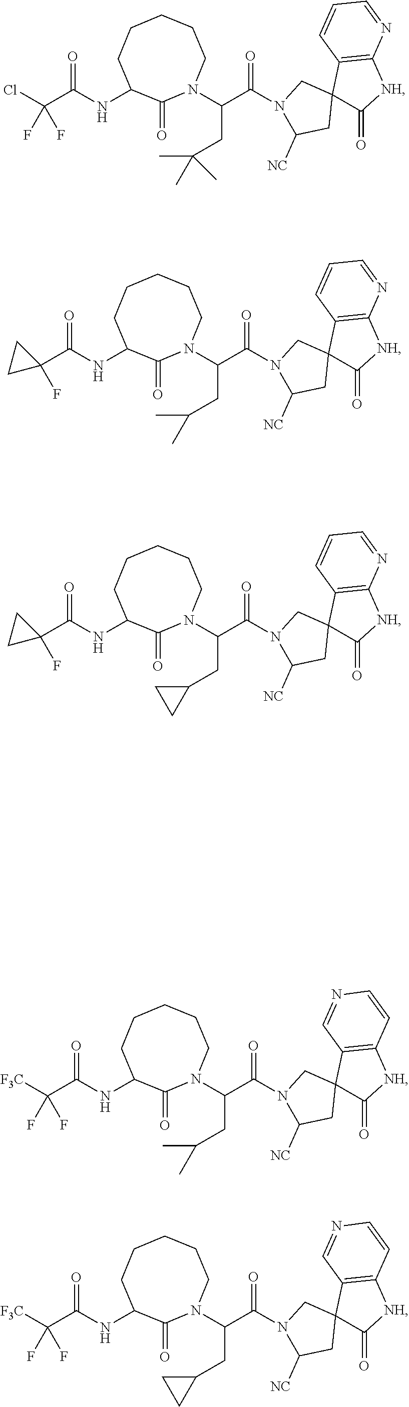 [SMILES: CC(C)(C)CC(C(=O)N1CC2(CC1C#N)C(=O)Nc1ncccc12)N1CCCCCC(NC(=O)C(F)(F)Cl)C1=O ; CC(C)CC(C(=O)N1CC2(CC1C#N)C(=O)Nc1ncccc12)N1CCCCCC(NC(=O)C2(F)CC2)C1=O ; N#CC1CC2(CN1C(=O)C(CC1CC1)N1CCCCCC(NC(=O)C3(F)CC3)C1=O)C(=O)Nc1ncccc12 ; CC(C)CC(C(=O)N1CC2(CC1C#N)C(=O)Nc1ccncc12)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)C(=O)Nc1ccncc12]

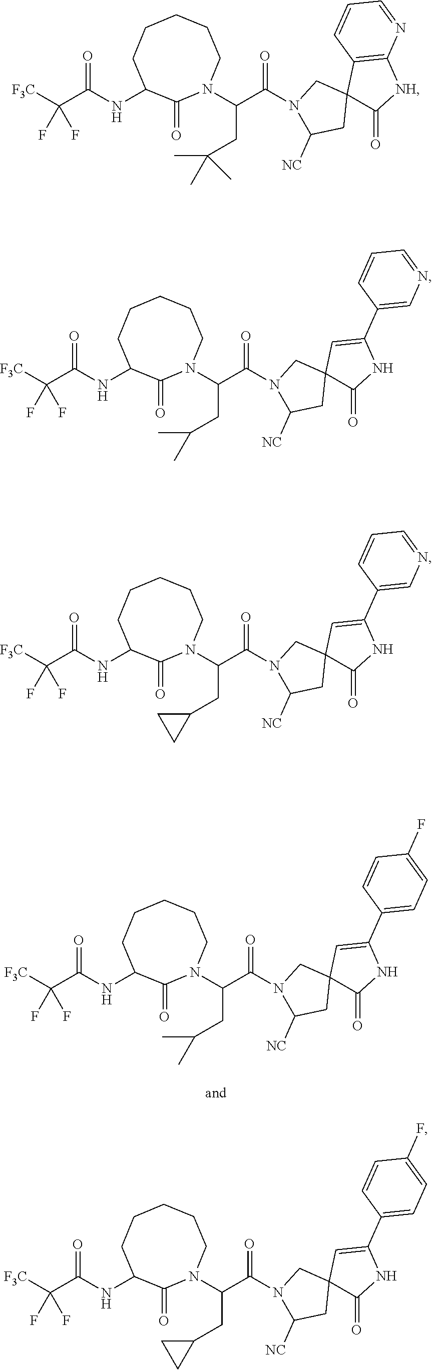 [SMILES: CC(C)(C)CC(C(=O)N1CC2(CC1C#N)C(=O)Nc1ncccc12)N1CCCCCC(NC(=O)C(F)(F)C(F)(F)F)C1=O ; CC(C)CC(C(=O)N1CC2(C=C(c3cccnc3)NC2=O)CC1C#N)N1CCCCCC(NC(=O)C(F)(F)C(F)(F)F)C1=O ; N#CC1CC2(C=C(c3cccnc3)NC2=O)CN1C(=O)C(CC1CC1)N1CCCCCC(NC(=O)C(F)(F)C(F)(F)F)C1=O ; CC(C)CC(C(=O)N1CC2(C=C(c3ccc(F)cc3)NC2=O)CC1C#N)N1CCCCCC(NC(=O)C(F)(F)C(F)(F)F)C1=O ; N#CC1CC2(C=C(c3ccc(F)cc3)NC2=O)CN1C(=O)C(CC1CC1)N1CCCCCC(NC(=O)C(F)(F)C(F)(F)F)C1=O]

or a pharmaceutically acceptable salt of any of the foregoing.

Additional examples of compounds of Formula (I), include the following:

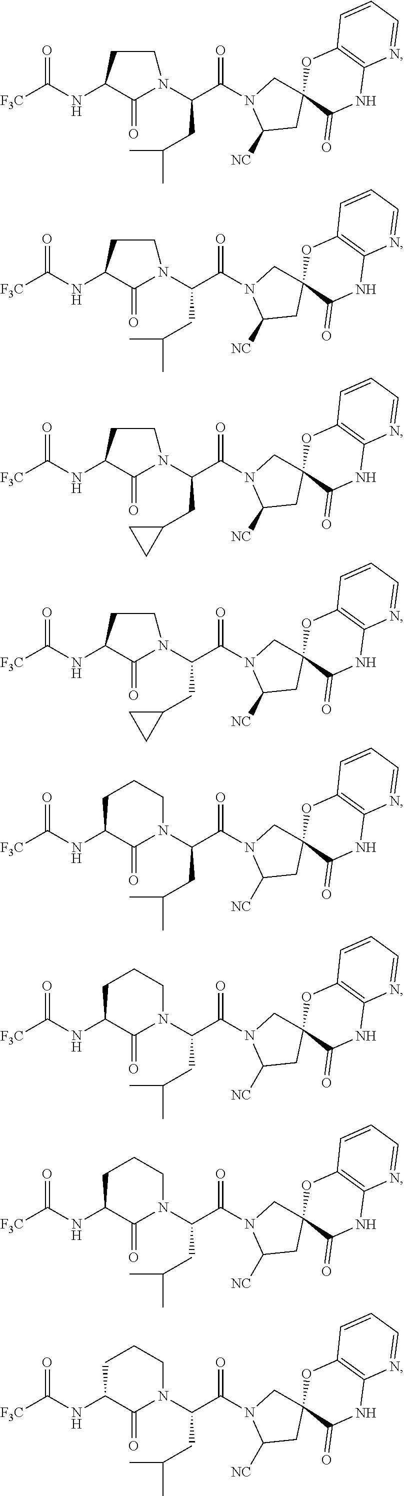

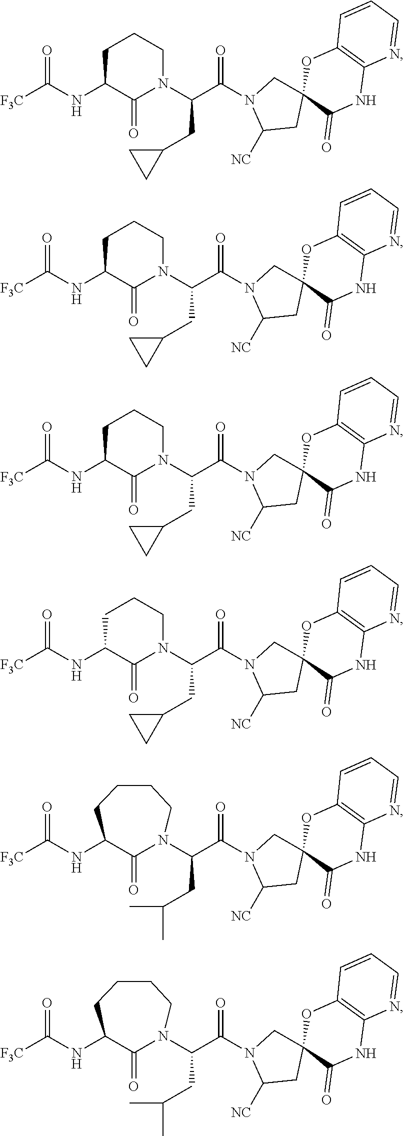

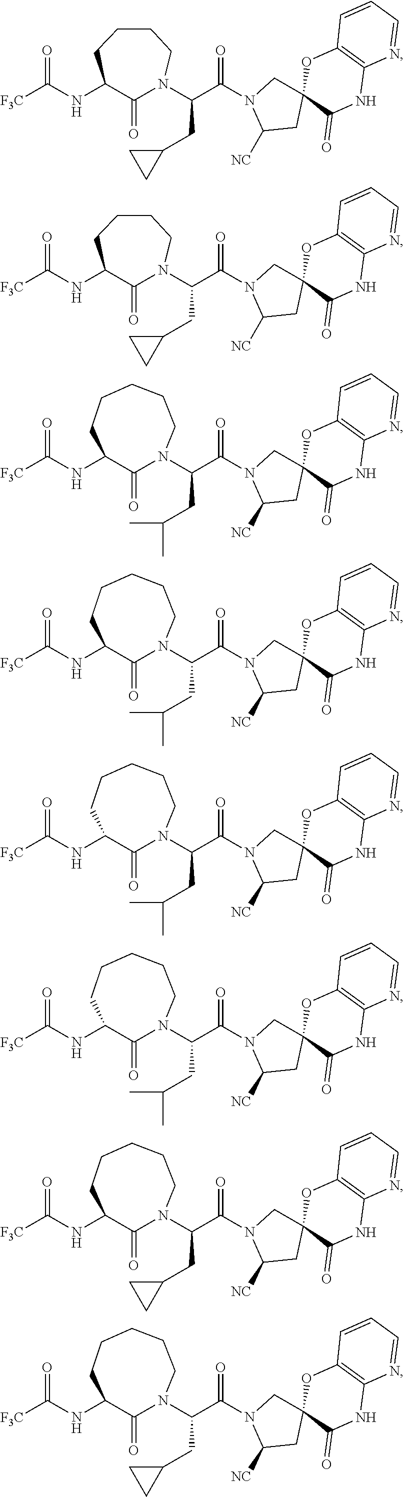

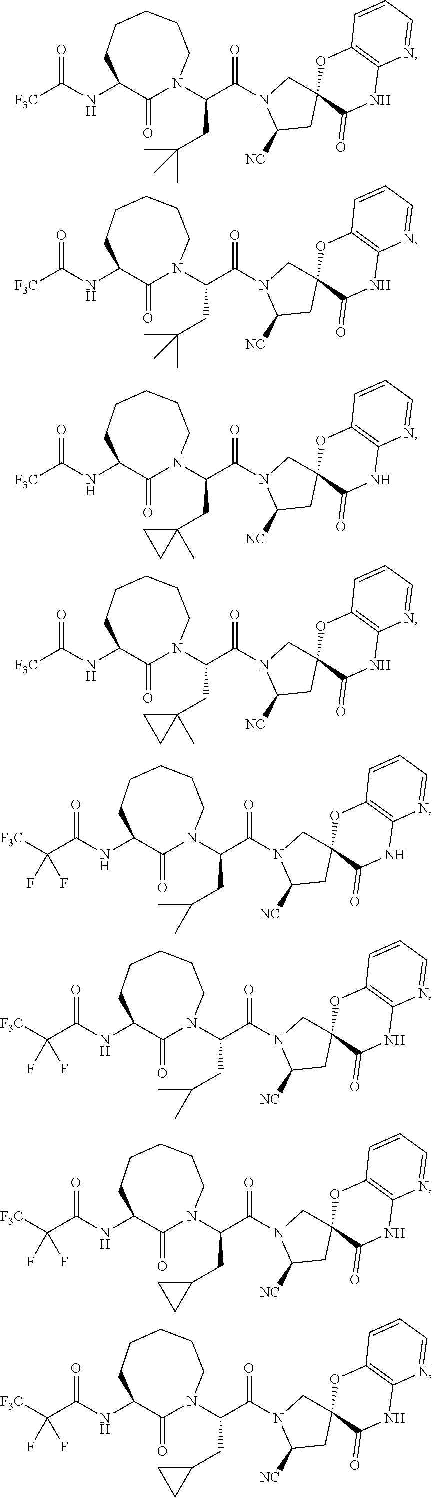

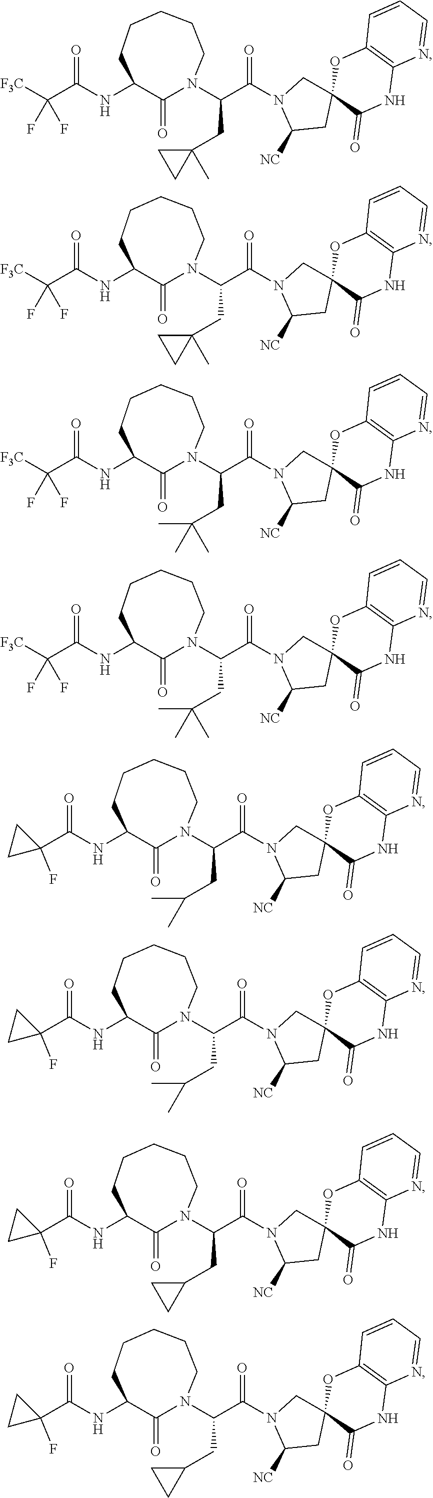

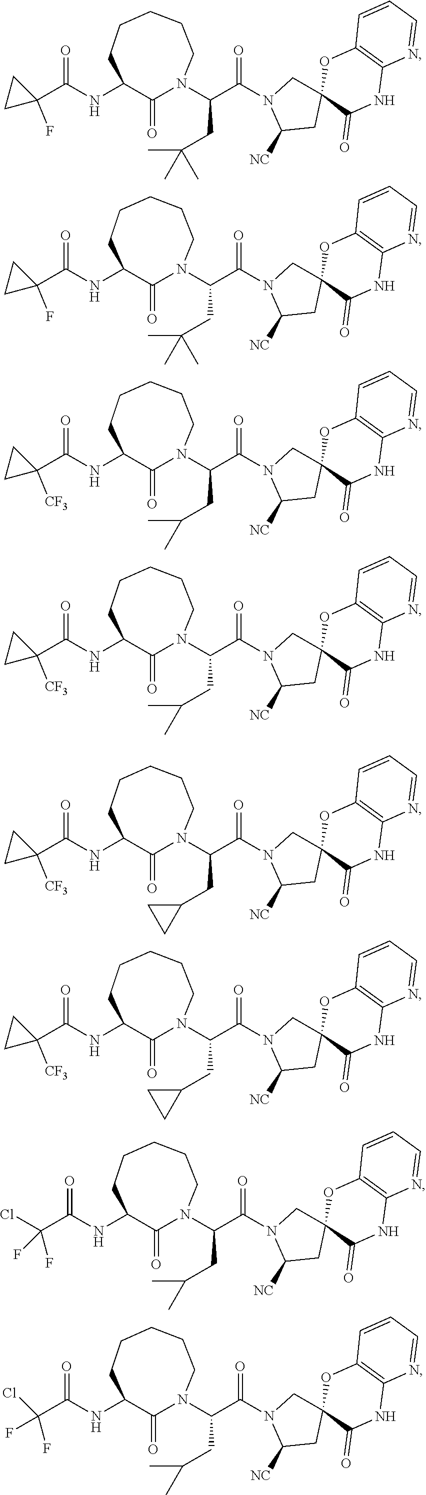

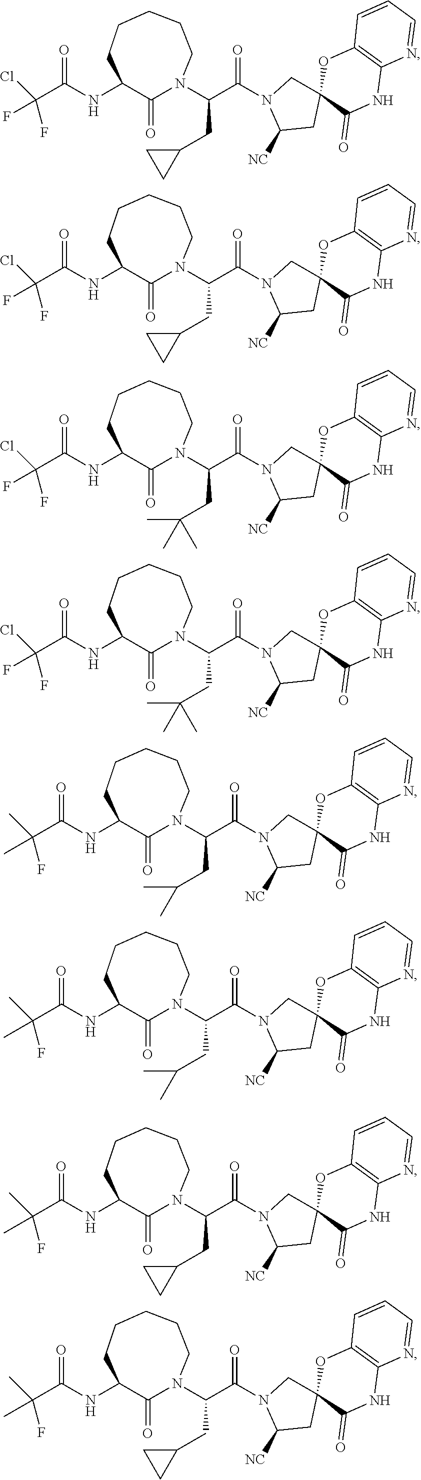

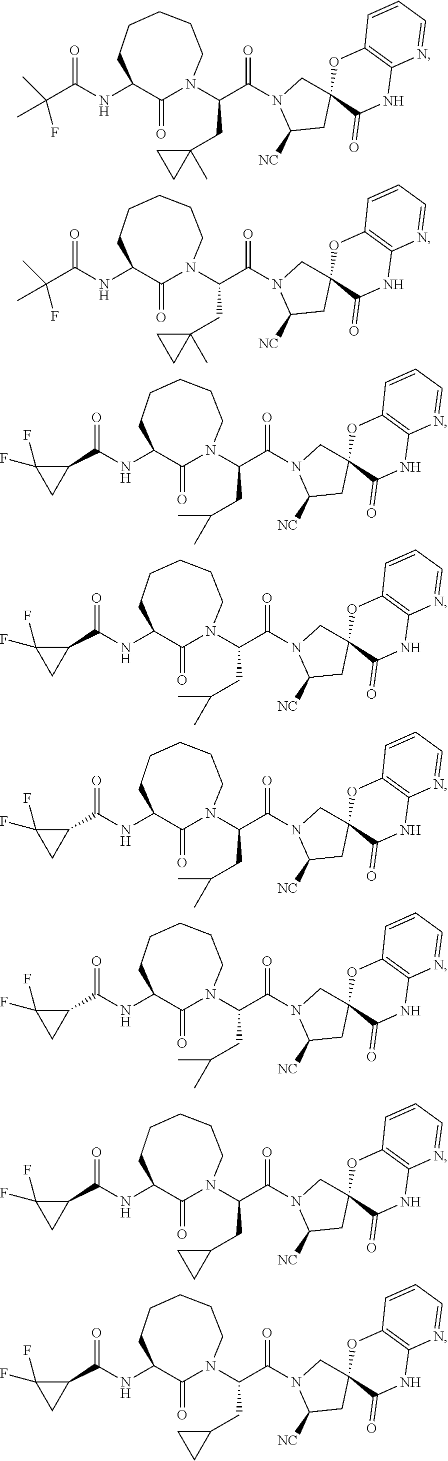

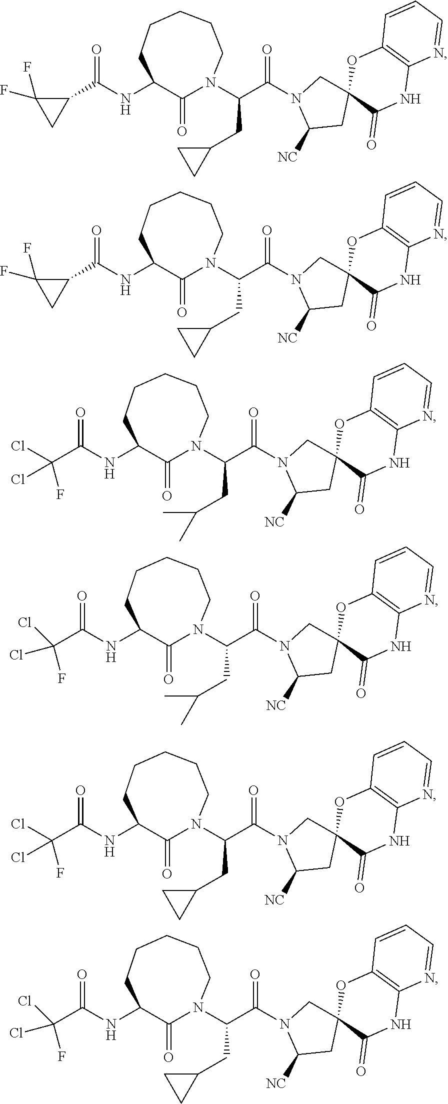

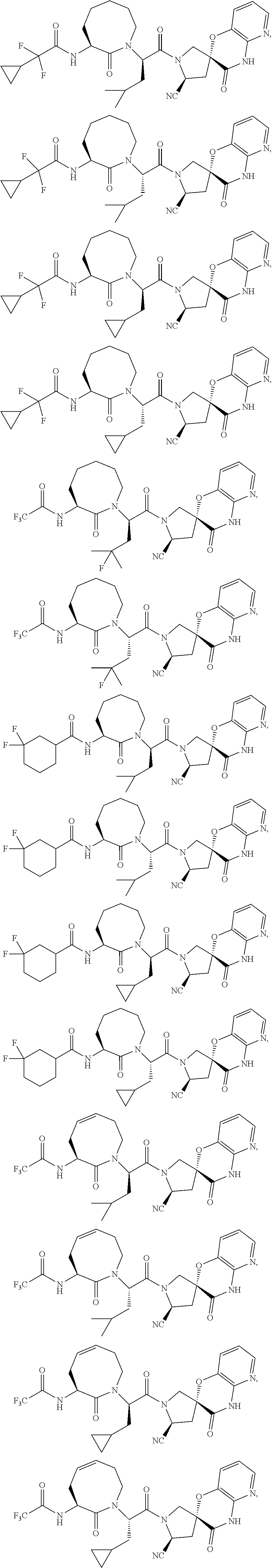

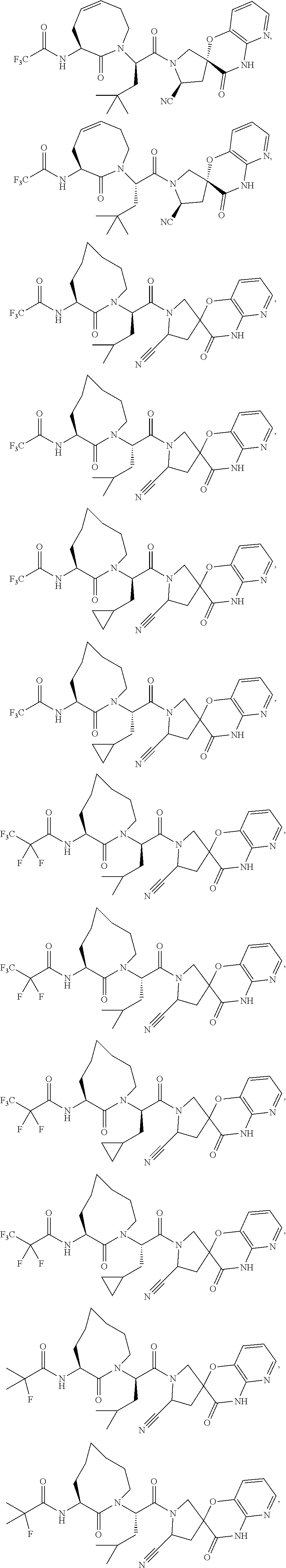

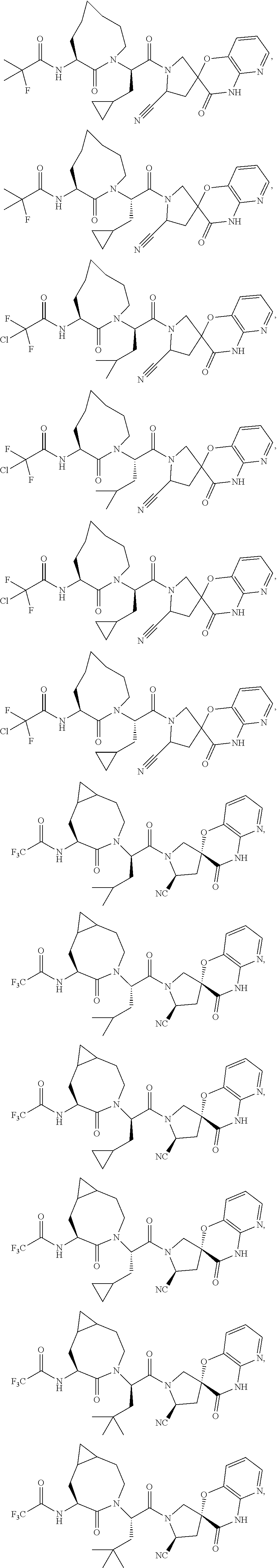

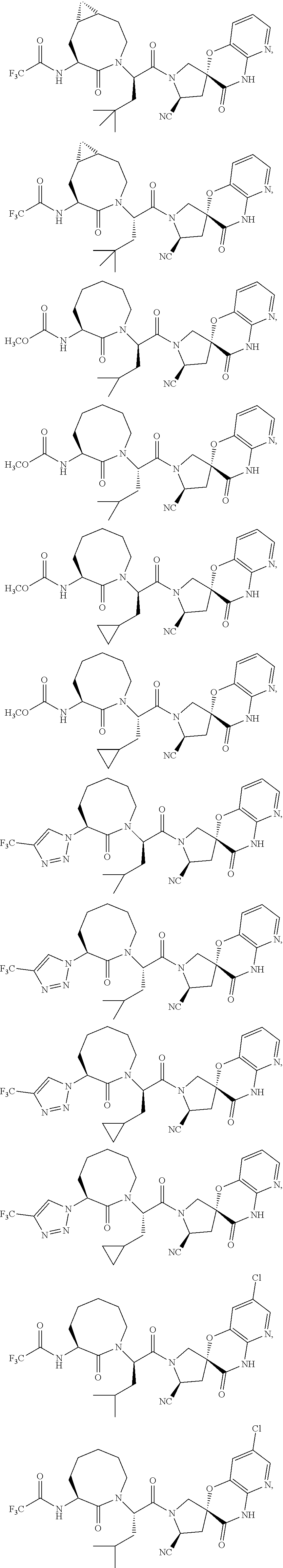

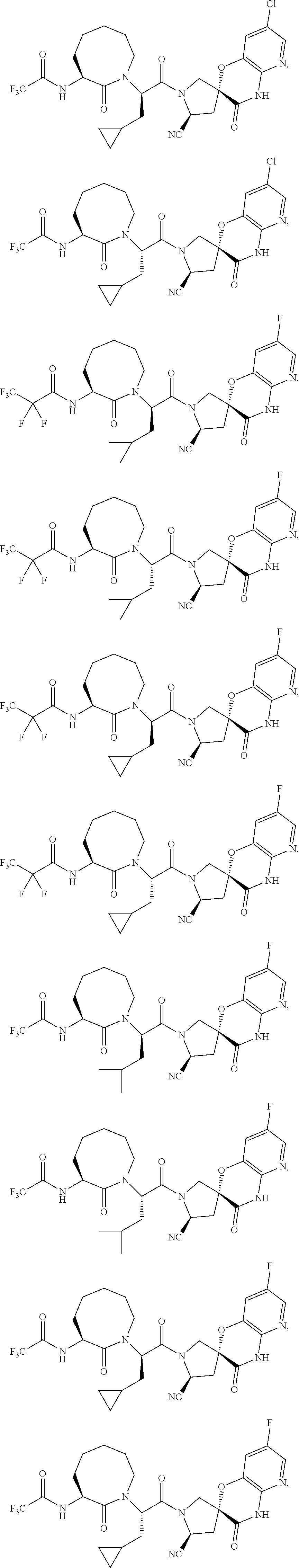

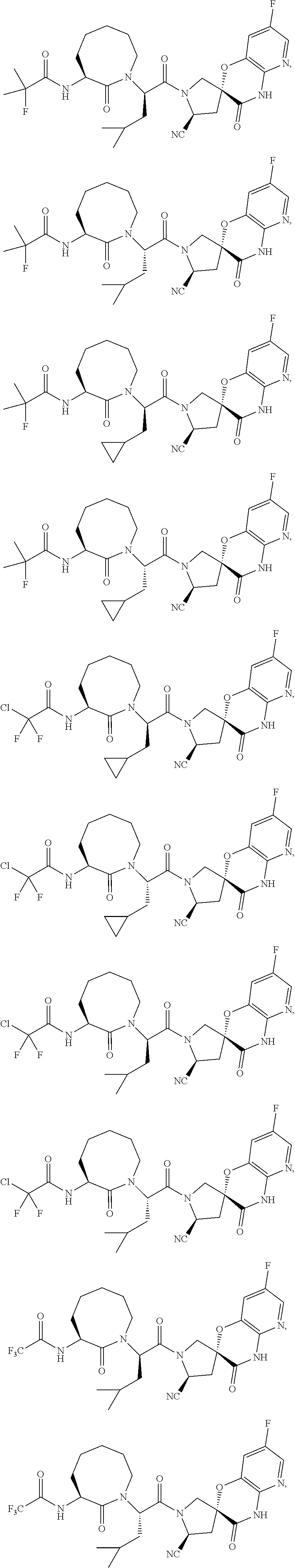

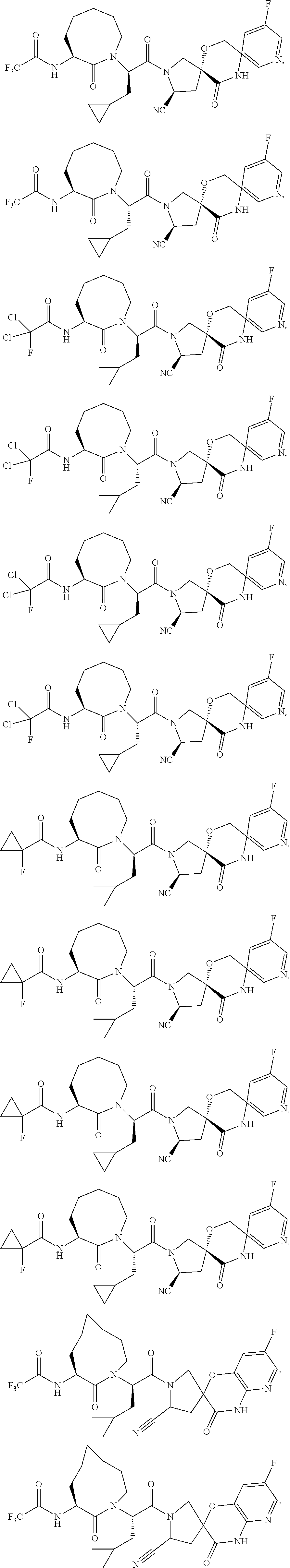

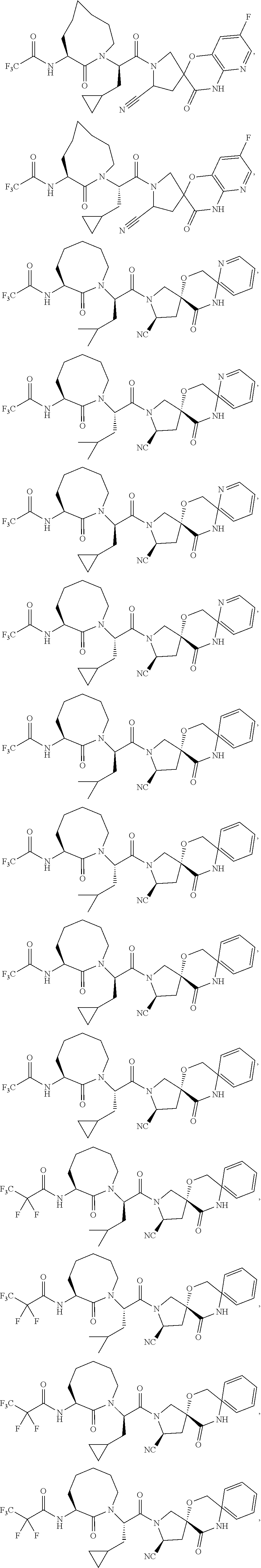

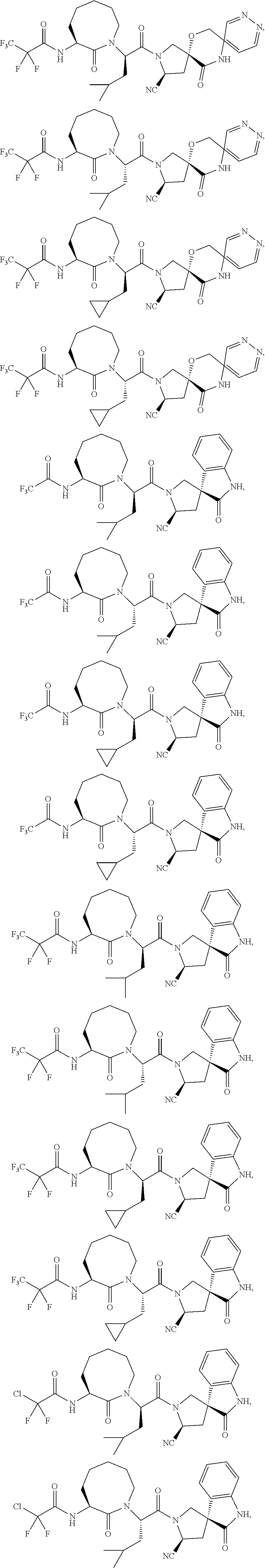

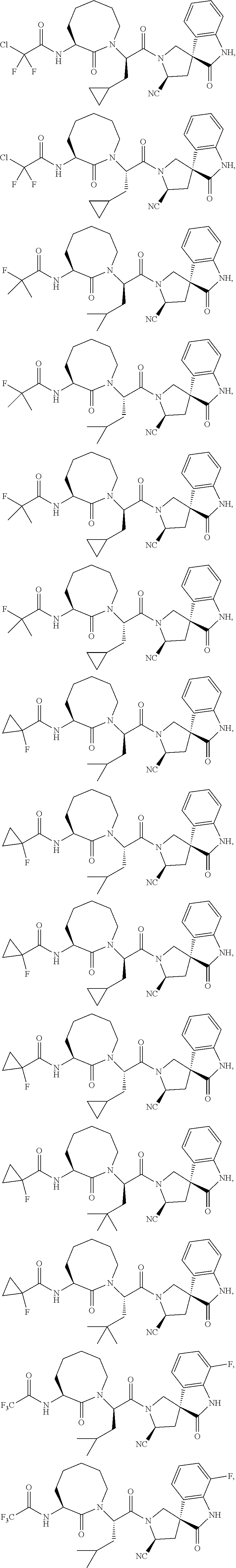

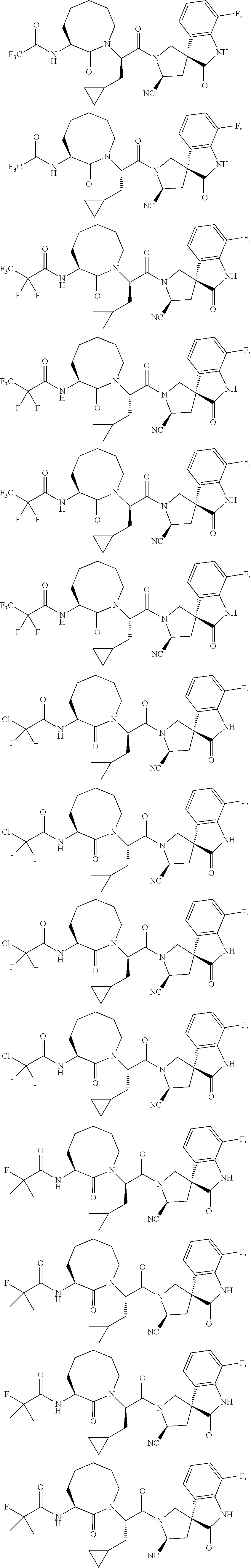

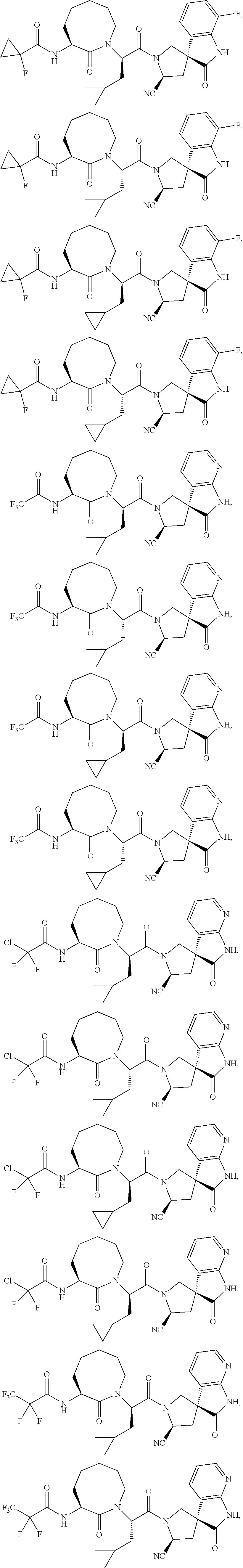

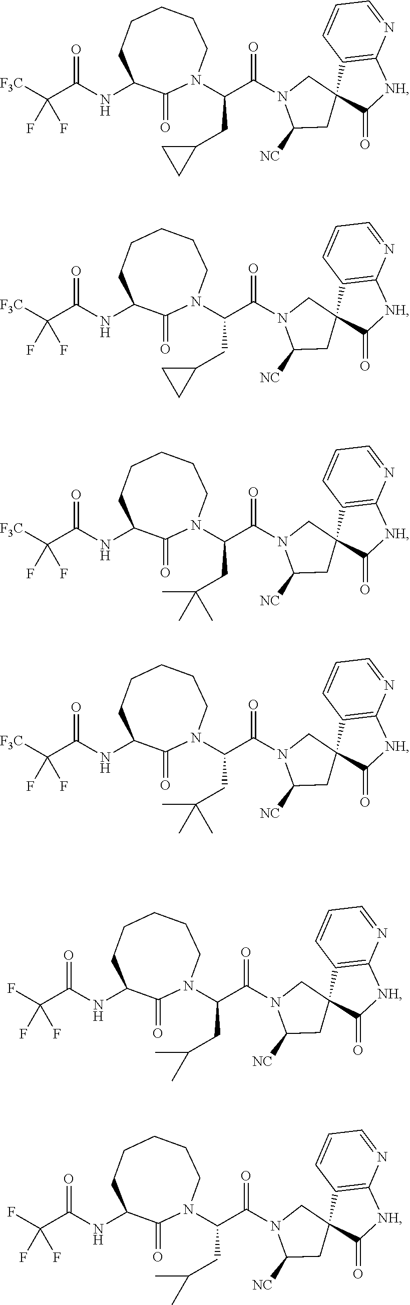

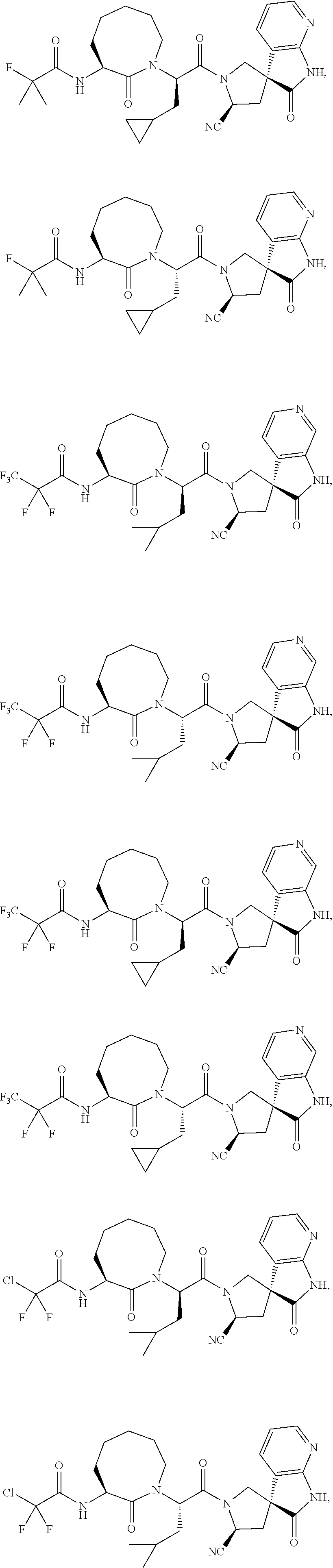

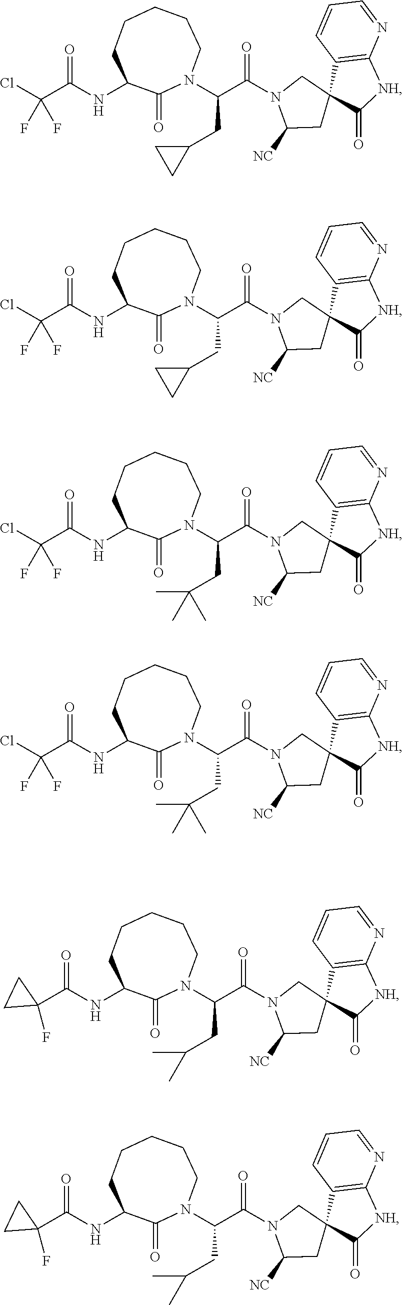

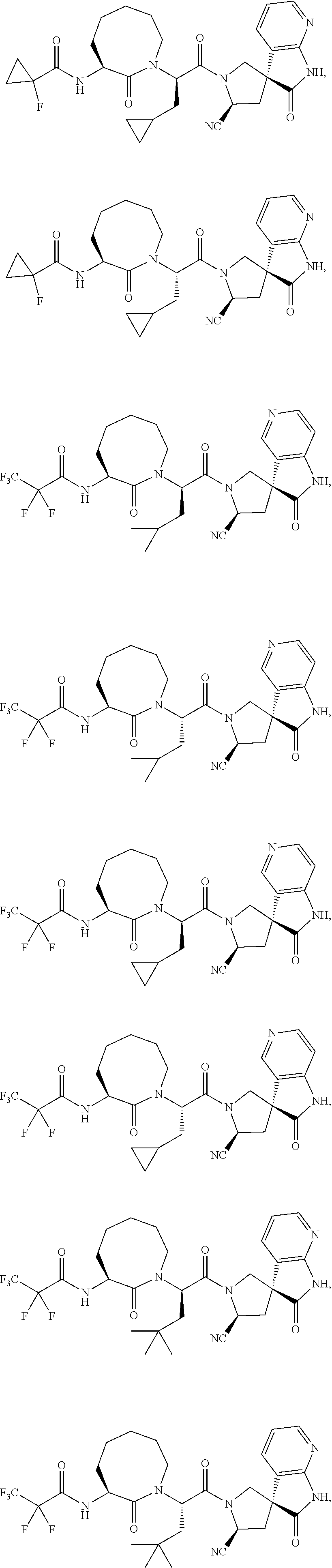

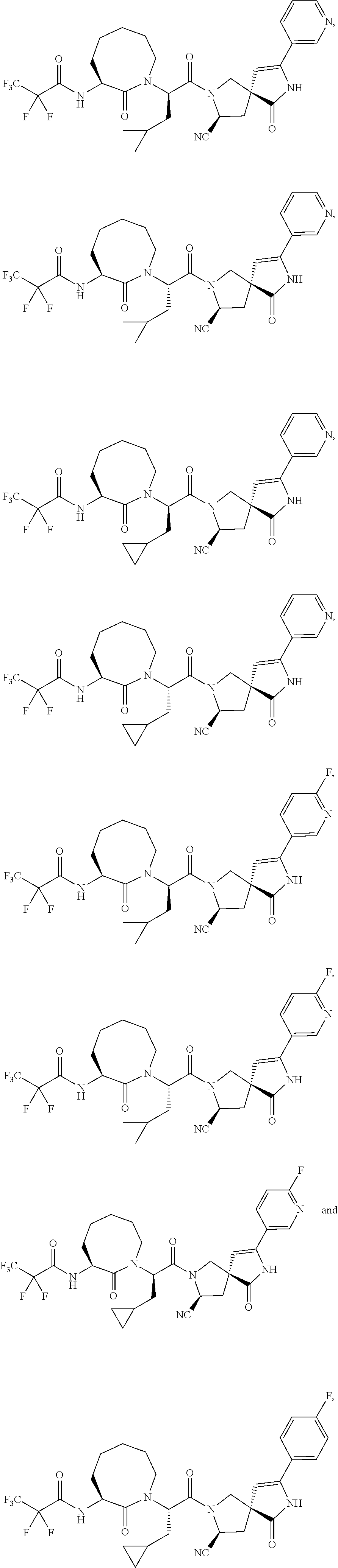

or a pharmaceutically acceptable salt of any of the foregoing.

### Synthesis

Compounds of Formula (I) along with those described herein may be prepared in various ways. General synthetic routes for preparing compounds of Formula (I) are shown and described herein along with some examples of starting materials used to synthesize compounds described herein. Additionally, for the purpose of the general synthetic routes, the structures depicted are appropriately protected, as known by one skilled in the art and the generic structures are meant to include these protecting groups. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

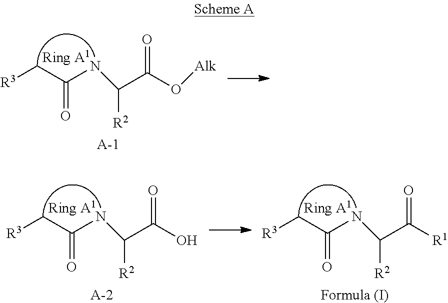

Scheme A describes the synthesis of compounds of general Formula (I). An ester of general Formula (A-1) (wherein Alk represents alkyl) can be hydrolyzed under basic conditions, for example when —OAlk is —OMe, using LiOH in MeOH, providing a compound of general Formula (A-2). Subsequent reaction of acids of general Formula (A-2), either by activating the carboxylic acid by converting it to an acid chloride, followed by reaction with the amino acid in the presence of a base, or by activation of the acid with a coupling reagent (such as HATU or T3P) followed by addition of the amine in the presence of a base (such as DIPEA), can result in a compound of general Formula (I).

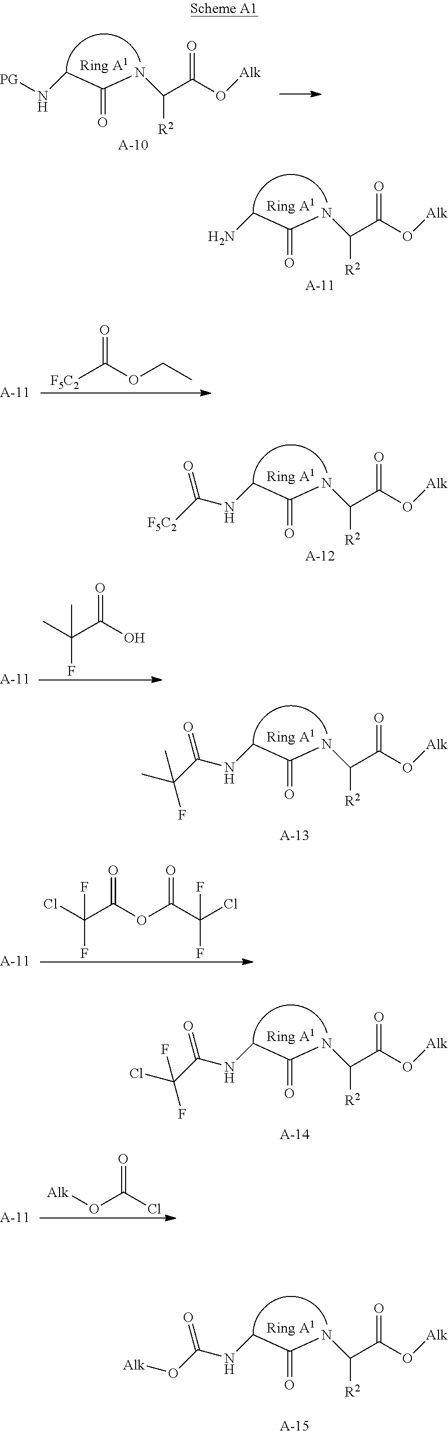

Scheme A1 describes how esters of Formula (A-10) can be used to obtain amido-esters of Formulae (A-12), (A-13) and (A-14) as examples for obtaining compounds of Formula (I), along with pharmaceutically acceptable salts thereof. A protected (PG) amine of Formula (A10) can be deprotected (e.g., if PG is boc, an acid, such as TFA, can be used to remove the PG). The free amine of Formula (A-10) can undergo transformation to an amide utilizing known amide formation conditions, for example, reaction of the amine in the presence of DIPEA at room temperature in methanol and an alkyl trihaloacetate, (such as ethyl 2,2-dichloro-2-fluoroacetate, methyl 2-chloro-2,2-difluoroacetate or ethyl 2-chloro-2,2-difluoroacetate) can afford Formula (A-12). Another example is HATU and triethylamine can be used to couple an acid to provide Formula (A-13). Formula (A-11) can also react with an acid chloride or an anhydride (as shown), with base in organic solvent, to provide the Formula (A-14). Alternatively, Formula (A-11) can react with a chloroformate to afford Formula (A-15). The synthesis of six to nine membered rings (Ring A¹) of Formula (A-10) has been described in the literature (e.g., Hoffman et al., Journal of Organic Chemistry (2003), 68(1):62-69 and Bioorganic & Medicinal Chemistry (2007) 15(3):1311-1322).

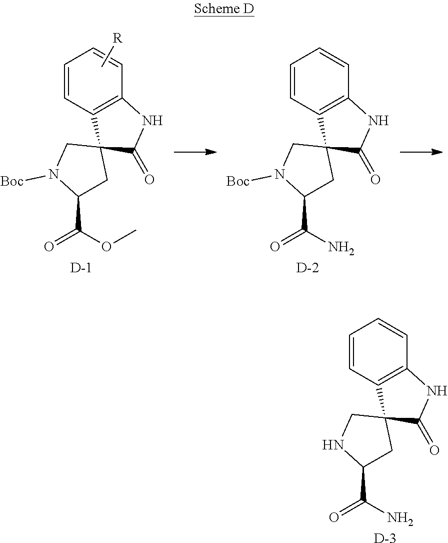

The synthesis of spirooxindole esters of Formula (D-1) can be prepared according to the literature (e.g., Efremov et al., Journal of Medicinal Chemistry (2012) 55(21):9069-9088).

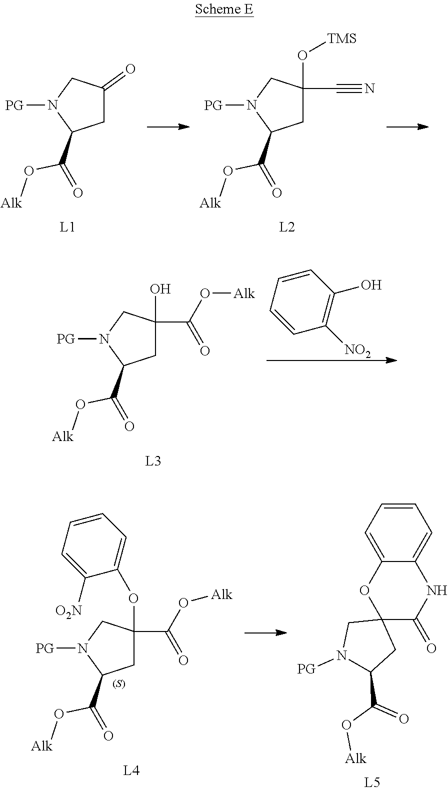

The synthesis of sprio-benzoxazinones of Formula (L5), depicted in Scheme E, can prepared with the conversion of a ketone of Formula (L1) to the protected cyanohydrin of Formula (L2) with the use of a silyl cyanide, such as TMSCN. Deprotection of the alcohol and alcoholysis of the CN group of compounds of Formula (L2) (for example, in HCl, CH₃OH) can lead to the formation of hydroxy-ester compounds of Formula (L3). Reaction with ortho-nitrophenols using typical Mitsunobu conditions can afford ether compounds of Formula (L4). Reduction of the nitro group of compounds of Formula (L4) can be accomplished under conditions known to those skilled in the art (e.g., Fe, NH₄Cl) in an alcoholic/aqueous solvent to afford the amine and concomitant ring closure to generate spirobenzoxazinones of Formula (L5), wherein the spirobenzoxazinone can be unsubstituted or substituted.

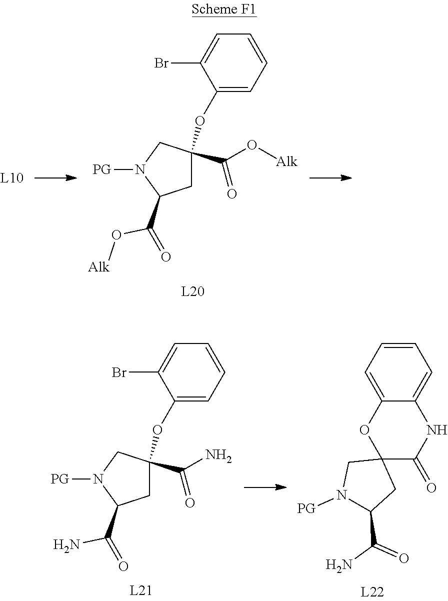

An alternative general synthetic method is depicted in Scheme F1. Compounds of Formula (L10) can be converted to compounds of Formula (L20) by reaction with an optionally substituted phenol in the presence of a base (such as NaOH) in an appropriate solvent (e.g., acetone). The two alkyl ester groups of Formula (L20) can be converted to the primary amides of Formula (L21) by heating with ammonia in a sealed vessel (facilitated when Alk is methyl or ethyl). Compounds of Formula (L21) can be reacted under copper catalysis conditions (e.g., CuI, DMEDA, Cs₂CO₃) to afford cyclization and provide compounds of Formula (L22), wherein the ring(s) can be unsubstituted or substituted.

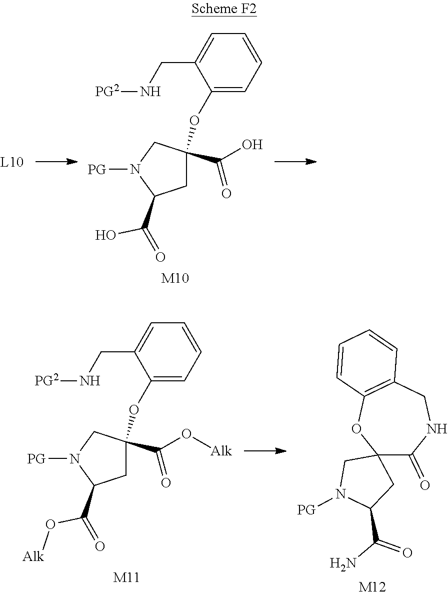

Similar to Scheme F1, Scheme F2 depicts a synthetic pathway towards seven-membered spiro rings. Compounds of Formula (L10) can be converted to compounds of Formula (M10) by reaction with an optionally substituted, optionally protected, 2-(aminomethyl)phenol in the presence of a base (such as NaOH) in an appropriate solvent (e.g., acetone) with excess base resulting in the diacid of Formula (M10). Alkylation of the acid groups utilizing an alkyl halide and base (e.g., CH₃I, K₂CO₃, DMF) can result in a diester of Formula (M11). The removal of the protecting group (PG²) either selectivity or unselectively over PG (protecting group) can afford a primary amine that, upon heating with base (e.g., triethylamine), will cyclize to form spirocyclyls of Formula (M12), wherein the spirocyclyls can be unsubstituted or substituted.

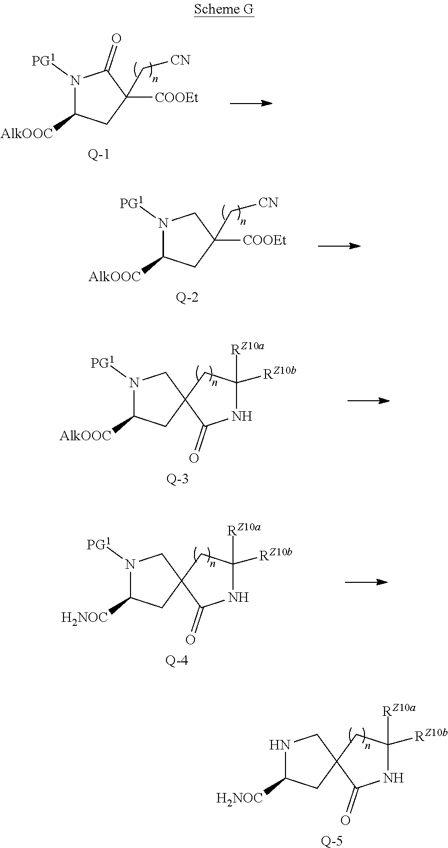

A spirolactam derivative of Formula (Q-5) can be prepared as provided in Scheme G. Pyrrolidinone of Formula (Q-1) when n is 1, Alk is Et and PG¹ is Boc can be prepared as described in Cowley et al., Org. Biomol. Chem. (2011) 9:7042-7056. Pyrrolidinone of Formula (Q-1) when n is 2, Alk is Et and PG¹ is Boc can be prepared by Michael reaction with acrylonitrile and 1-(t-butyl) 2,4-diethyl (2S)-5-oxopyrrolidine-1,2,4-tricarboxylate synthesized as described in Cowley et al., Org. Biomol. Chem. (2011) 9:7042-7056. Selective reduction of a pyrrolidinone of Formula (Q-1) (with PG¹ representing a suitable nitrogen protecting group, for example, -Boc) using lithium triethylborohydride followed by further reduction of the hemiaminal intermediate with triethysilane and boron trifluoride etherate (Dorta et al., Tetrahedron Lett. (1994) 35(13):2053-2056) can provide a pyrrolidine of Formula (Q-2). Nitrile reduction (for example, with CoCl₂ and NaBH₄), and subsequent cyclisation in-situ can provide a lactam of Formula (Q-3). Alternatively, a nitrile of Formula (Q-2) can be converted via a Kulinkovich-Szymoniak reaction into a cyclopropyl amine, which can react with the ethyl ester in-situ to afford a lactam of Formula (Q-3). A primary amide of Formula (Q-4) can be prepared by aminolysis of an ester of Formula (Q-3), when Alk is methyl or ethyl, or by ester hydrolysis in basic conditions followed by the reaction with ammonia under typical amide coupling conditions. Protecting group removal (for example, when PG¹ is Boc, by treatment with HCl) can provide an amine of Formula (Q-5). The spirolactam intermediates can be resolved using various techniques (such as purification by chromatography).

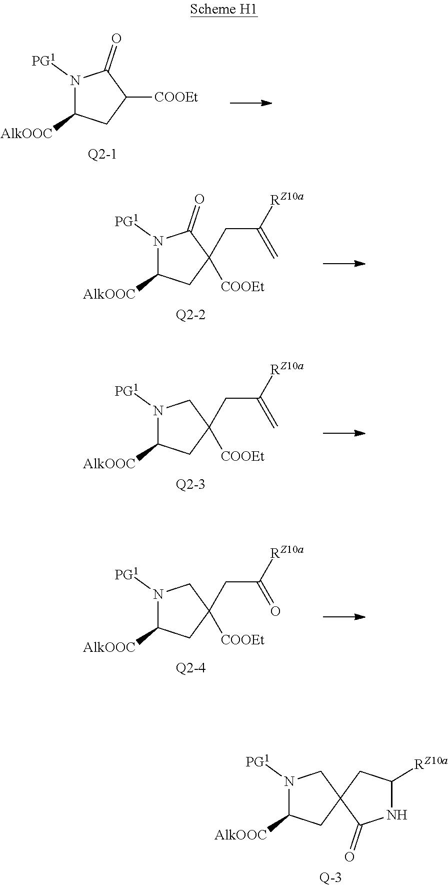

Alternatively, lactams of Formula (Q-3) when n is 1, R^{z10b} is H and PG¹ is Boc can be prepared as provided in Scheme H1. Alkylation of a pyrrolidinone of Formula (Q2-1) (synthesized as described in Cowley et al., Org. Biomol. Chem. (2011) 9:7042-7056) with an allyl halide in presence of a base (such as sodium hydride) can provide allyl of Formula (Q2-2). A pyrrolidine of Formula (Q2-3) can be prepared by reduction using lithium triethylborohydride followed by treatment with triethysilane and boron trifluoride etherate as described for the pyrrolidine of Formula (Q-2). A ketone of Formula (Q2-4) can be prepared by oxidative cleavage using, for example, osmium tetroxide and sodium periodate. Reductive amination with ammonium acetate and a reducing agent (such as sodium cyanoborohydride followed by cyclisation in-situ) can provide a lactam of Formula (Q-3).

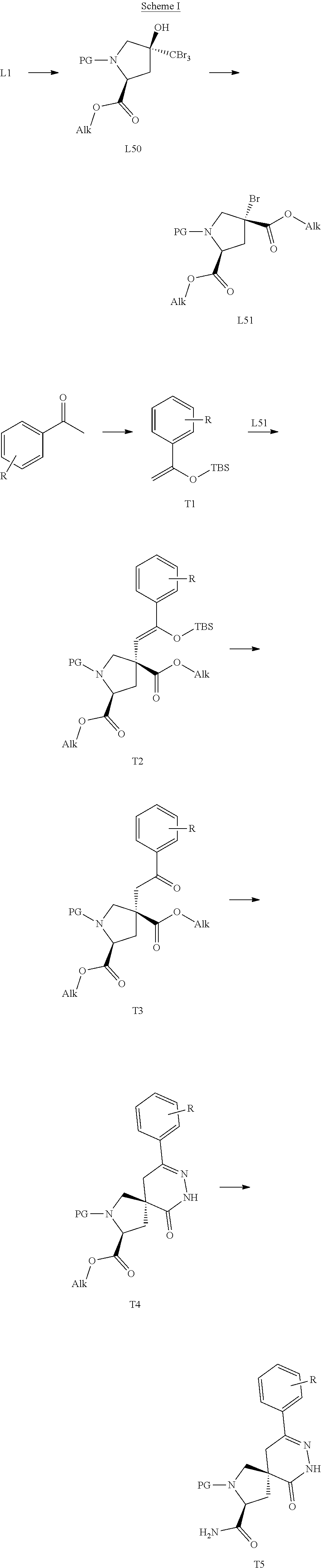

A general synthesis towards spiropyridazinones is provided in Scheme I. Acetophenones can be converted to an enol ether of Formula (T1) using methods known to those skilled in the art (for example, TBSCl, NaI, triethylamine in CH₂Cl₂). In parallel, a compound of Formula (L50) can be prepared using a similar procedure for the formation of a compound of Formula (10) with the exception that bromoform can be used instead of chloroform. Compounds of Formula (L50) can be transformed into compounds of Formula (L51) using a base in an alcoholic solvent (such as DBU in methanol). Compounds of Formula (L51) can react with an enol ether of Formula (T1) using a copper catalyst and base to form compounds of Formula (T2). Deprotection of the enol ether, for example, with TBAF in THF, can afford a ketone of Formula (T3), which can be reacted with hydrazine (in an organic solvent, with optional heating) to afford a compound of Formula (T4). The ester of Formula (T4) can be converted to a primary amide of Formula (T5) via addition of concentrated ammonia in an organic solvent, optionally heated under pressure.

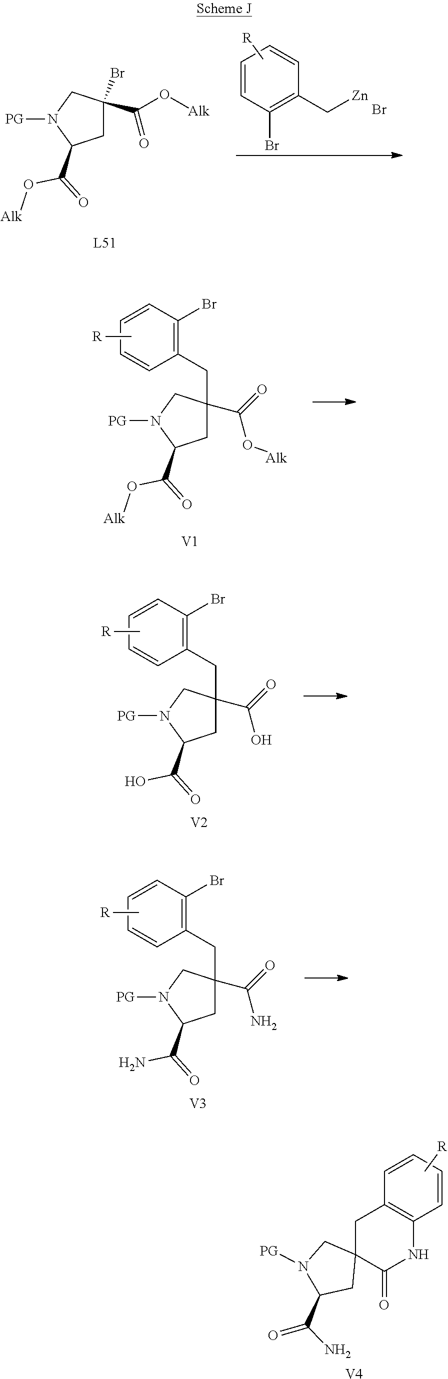

A general synthesis to prepare spirolactams is shown in Scheme J. Starting bromides of Formula (L51) can be reacted with a (2-bromobenzyl) zinc bromide via a cobalt catalyst in an organic solvent to afford a compound of Formula (V1). Subsequent conversion of a compound of Formula (V1) to the bis-acid of Formula (V2) can be accomplished via basic hydrolysis. A bis-amide of Formula (V3) can be formed by reacting the bis acid of Formula (V2) with ammonia and a coupling catalyst. Ring closure via copper catalysis can afford a compound of Formula (V4).

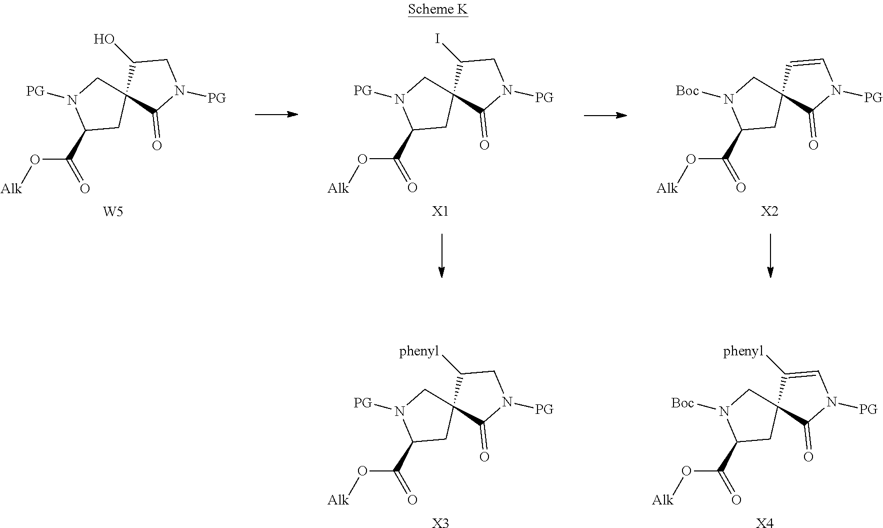

Scheme K provides a general synthesis of phenyl substituted spirolactams. The alcohol of a compound of Formula (W5) can be exchanged for a halogen (for example, iodine) via the Appel reaction using iodine and triphenylphosphine. An alkyl iodide of Formula (X1) can undergo a Suzuki reaction with a phenyl borane or phenyl boronic acid, using procedures described known to those skilled in the art to afford a compound of Formula (X3). Alternatively, the halogen of a compound of Formula (X1) can be eliminated to form a double bond compound of Formula (X2). Compounds of Formula (X2) can undergo a Heck-type coupling reaction using methods known to those skilled in the art to afford a compound of Formula (X4).

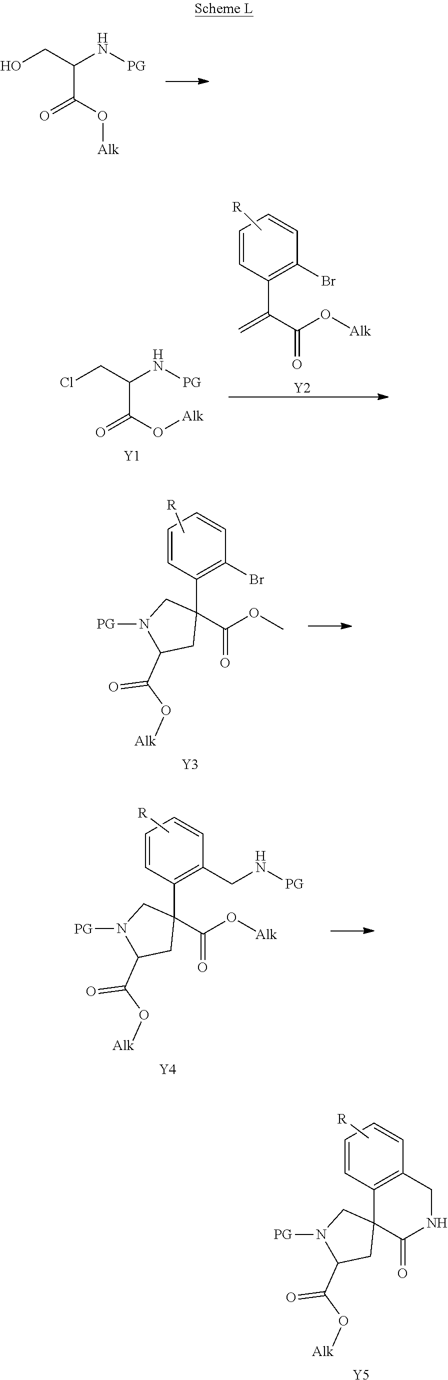

Depicted in Scheme L is a general synthesis towards spirolactams of Formula (Y5). Commercially available protected serine esters can be converted to an alkyl chloride of Formula (Y1) using the Appel reaction (PPh₃, CCl₄). A compound of Formula (Y1) can undergo a cycloaddition with an aryl acrylate of Formula (Y2) to afford a cyclic compound of Formula (Y3). Subsequent formation of a compound of Formula (Y4) can be accomplished via a Suzuki coupling of a vinyl potassium trifluoroborate with the aryl bromide of Formula (Y3). Alternatively, formation of a compound of Formula (Y4) can be accomplished by reaction of the aryl bromide of Formula (Y3) with Zn(CN)2 with the aid of a Pd catalyst, followed by reduction of the CN group to afford a compound of Formula (Y4) where PG is hydrogen. Compounds of Formula (Y4) can be cyclized by deprotection of the amine, followed by heating, or alternatively, deprotection of the amine and the ester using a coupling agent to afford a spirocyclic lactam of Formula (Y5).

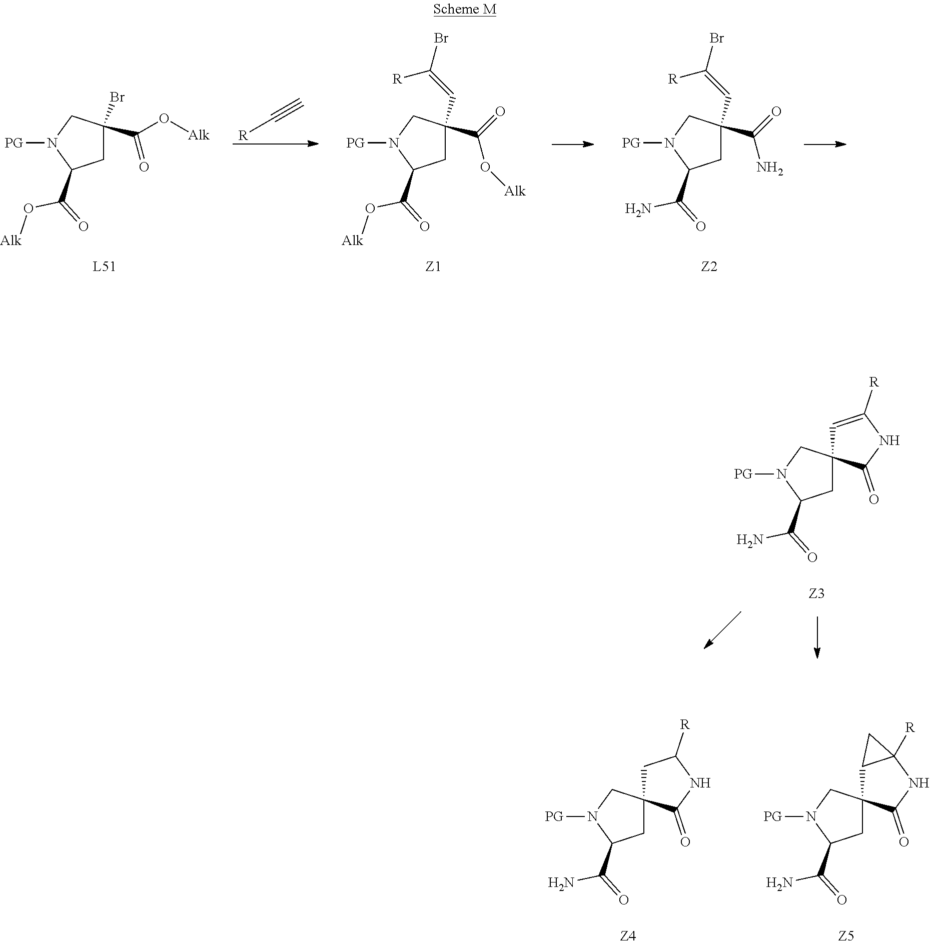

Depicted in Scheme M is a general synthetic pathway towards spirolactams of Formula (Z4) and Formula (Z5) where R can be C₁-C₆ alkyl, C₁-C₆ alkoxy, aryl or heteroaryl. Compounds of the Formula (L51) can react with an acetylene, mediated by copper (e.g. CuBr) in the presence of a base (such as triethylamine) to afford compounds of Formula (Z1). Subsequent conversion of the ester groups to primary amides can be affected by the addition of ammonia in methanol (MeOH) in a sealed reactor to give compounds of Formula (Z2). Ring closure, to form compounds of Formula (Z3), can be mediated by copper or palladium catalysis (for example, Xphos Pd G3). Compounds of Formula (Z3) can be subjected to hydrogen and a palladium catalyst (such as Pd/C) to reduce the double bond to afford compounds of Formula (Z4). Alternatively, compounds of Formula (Z3) can be further derivatized in a Simmons-Smith reaction or carbene insertion to afford compounds of the Formula (Z5).

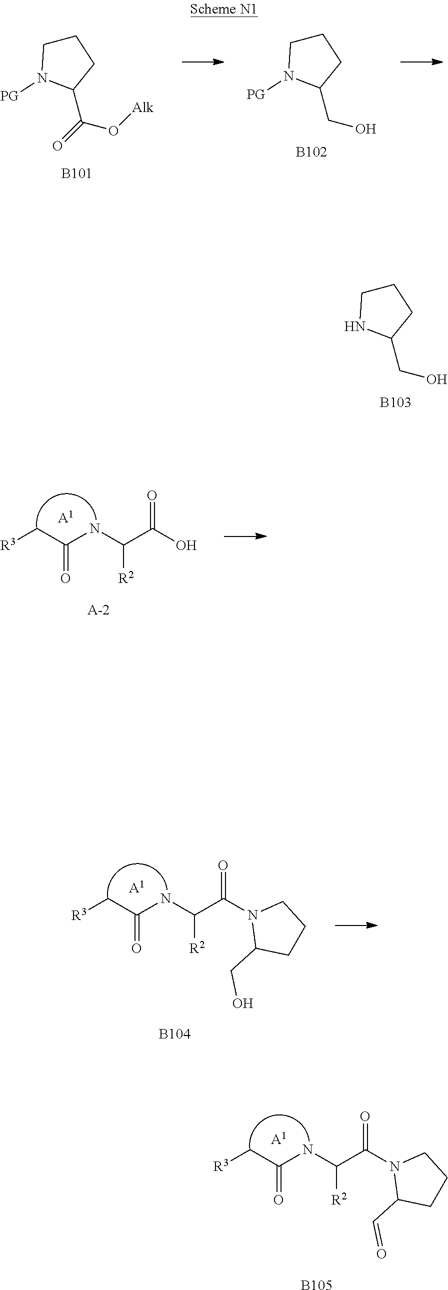

In Schemes N1-N8, the pyrrolidinyl ring shown in each of the structures corresponds to the pyrrolidinyl ring that has R⁴ attached in each of the R¹ groups. Additionally, Schemes N1, N2 and N4-N8, “A¹” denotes Ring A¹ as provided in Formula (I). Scheme N depicts a general synthetic method to form an aldehyde of Formula (B105) where a sub-structure of R¹ is depicted and the chemical modifications described here can be applied to other R¹ groups described herein. An ester of Formula (B101) can be reduced to the alcohol of Formula (B102) using methods provided in the literature (e.g., LiBH₄). Removal of the protecting group (PG) can afford a compound of the Formula (B103) (e.g., HCl when PG is Boc). The amine of Formula (B103) can be coupled to a carboxylic acid of Formula (A-2) using known coupling agents (e.g., HATU) to afford a compound of Formula (B104). Oxidation of the alcohol group of Formula (B104) can be accomplished using an oxidizing reagent described in the literature (such as IBX or Dess-Martin periodinane) to provide an aldehyde of Formula (B105).

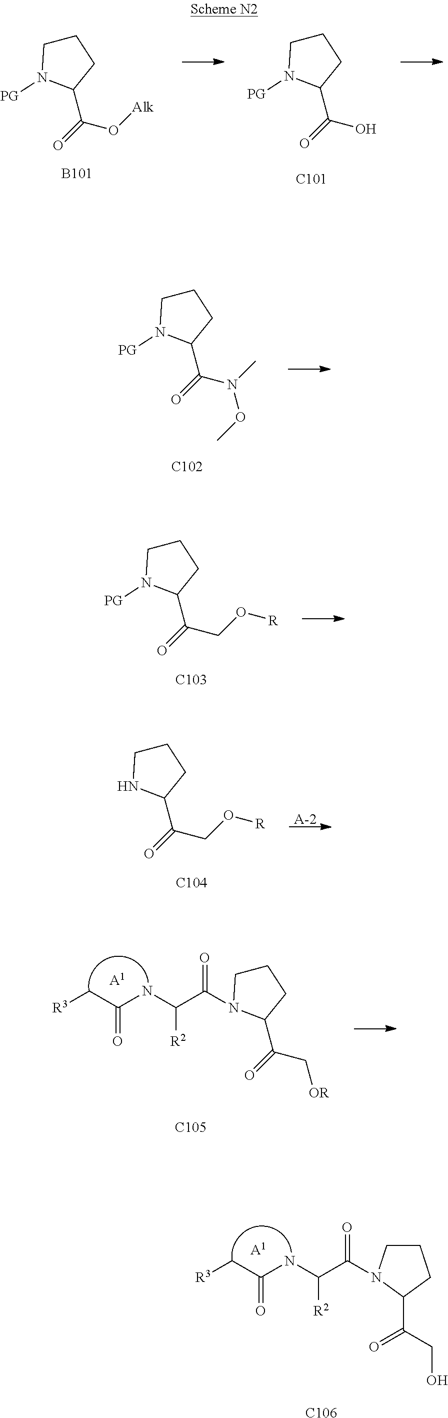

Scheme N2 depicts a general method to synthesize the hydroxyketones of Formula (C106) where a sub-structure of R¹ is depicted and the chemical modifications described here can be applied to other R¹ groups described herein. An ester of Formula (B101) can be hydrolyzed, for example, with LiOH when Alk is methyl, to afford a carboxylic acid of Formula (C101). Conversion of compounds of Formula (C101) to an amide of Formula (C102) can be accomplished by using a coupling agent (e.g., BOP, HATU, etc.) in the presence of N,O-dimethylhydroxyamine and a base (such as triethylamine) in an appropriate solvent (e.g., DMF). Addition of an organometallic reagent to the Weinreb amide of Formula (C102), followed by work-up, can result in a ketone of Formula (C103). An example, wherein R is benzyl, is the formation of an organometallic reagent by mixing Mg, HgCl₂ and benzylchloromethyl ether, followed by addition to a Weinreb amide of Formula (C102), followed by work-up with saturated ammonium chloride, (See Evans et al., Journal of the American Chemical Society (1988) 110(11):3560-3578 and Mendonca et al., Bioorganic & Medicinal Chemistry Letters (2002) 12(20):2887-2891) to afford an ether of the Formula (C103). Removal of the nitrogen protecting group (PG), (for example, using HCl or pTSA when PG is Boc) can afford a compound of Formula (C104). Subsequent coupling of a compound of Formula (C104) with a carboxylic acid of a compound of Formula (A-2), using a coupling agent (e.g., TCFH, HBTU, etc.) can afford a compound of Formula (C105). The R group can be selectively removed (for example, by catalytic hydrogenation conditions (when R is benzyl (Bn), in Pd/C in a hydrogen atmosphere)) and provide a hydroxyketone of Formula (C106).

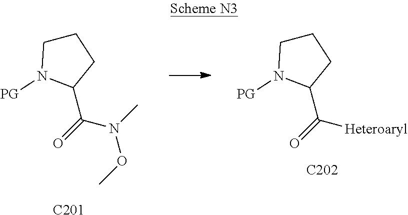

Another method for preparing an intermediate that can be used to prepare R¹ is shown in Scheme N3 where a sub-structure of R¹ is depicted and the chemical modifications described here can be applied to other R¹ groups described herein. In Scheme N3, the Weinreb amide of Formula (C201) can be converted to a heterocyclic ketone of Formula (C202) using methods known to those skilled in the art (for example, Nahm et al., Tetrahedron Lett. (1981) 22(39), 3815-3818 and Balasubramaniam et al., Synthesis (2008) 23:3707-3738).

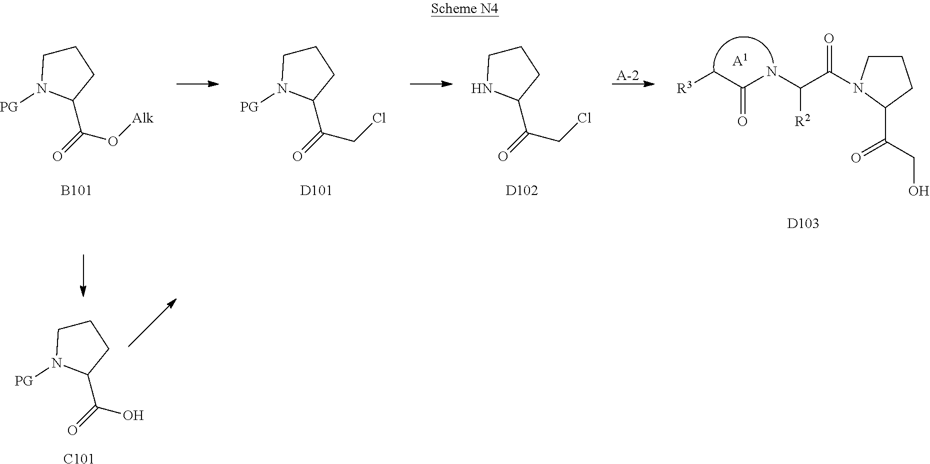

A general synthetic method to afford chloromethylketones of Formula (D103) is depicted in Scheme N4 where a sub-structure of R¹ is depicted and the chemical modifications described here can be applied to other R¹ groups described herein. Compounds of Formula (B101) can be converted to a chloromethylketones of Formula (D101), for example, when Alk is methyl or ethyl, via methods known in the art (e.g., Pace et al., Advanced Synthesis & Catalysis (2013) 355(5):919-926 and Concellon et al., Journal of Organic Chemistry (2001) 66(25):8661-8665) using chloroiodomethane, or bromoiodomethane, and a strong base (such as LDA). An ester of Formula (B101) can be hydrolyzed to a compound of Formula (C101) and then converted to a chloromethylketone employing a variety of methods known in the art, including, but not limited to, the following: isopropyl chloroformate, 4-methylmorpholine, then diazomethane (See Sun et al., J. Med. Chem. 2006, 49(11):3153-3158). The protecting group of compounds of Formula (D101) can be cleaved to afford an amine of Formula (D102). Subsequent coupling of an amine of Formula (D102) with a carboxylic acid of Formula (A-2) can be utilized using known coupling agents to provide chloromethylketones of Formula (D103).

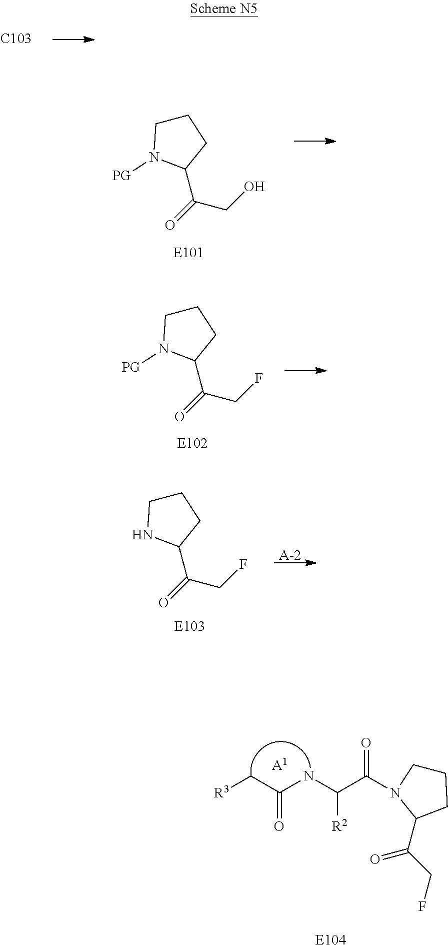

A general synthesis towards fluoromethylketone compounds of Formula (E104) is provided in Scheme N5 where a sub-structure of R¹ is depicted and the chemical modifications described here can be applied to other R¹ groups described herein. The benzyl ether of Formula (C103) can be cleaved by Pd catalyzed hydrogenolysis (for example Pd/C in methanol) to afford a compound of Formula (E101). Subsequent conversion of an alcohol of Formula (E101) can be converted to a fluoromethylketone using fluorinating reagents that are known to those skilled in the art (e.g., a sulfonyl fluoride/HF-Et₃N) to afford a fluoromethylketone of Formula (E102). Deprotection of the protecting group (PG) (for example, with HCl if PG is Boc) can afford compounds of Formula (E103). Compound of Formula (E103) can be then coupled with a carboxylic acid of Formula (A-2) using a wide variety of commercially available coupling agents (such as TCFH in DMF with N-methyl imidazole) to afford a fluoromethylketone of Formula (E104). Alternatively, fluoromethylketone compounds of Formula (E102) can be obtained from a carboxylic acid of Formula (C101) via a modified Daikin-West reaction using fluoroacetic anhydride, triethylamine and DMAP in benzene (See Rasnick, D., Anal. Biochem. (1985) 149:461-465).

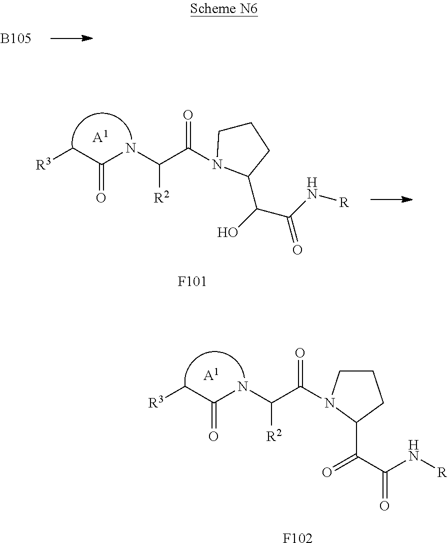

A general synthesis method towards a-ketoamides of Formula (F102) is described in Scheme N6 where a sub-structure of R¹ is depicted and the chemical modifications described here can be applied to other R¹ groups described herein. The starting aldehyde of Formula (B105) can be treated with an isocyanide under conditions described in the literature to afford an alpha-hydroxyamide of Formula (F101). Subsequent oxidation of the alcohol group of a compound of Formula (F101) utilizing procedures known in the literature (like the Swern or Dess-Martin periodinane oxidation) can afford the alphaketoamide of Formula (F102).

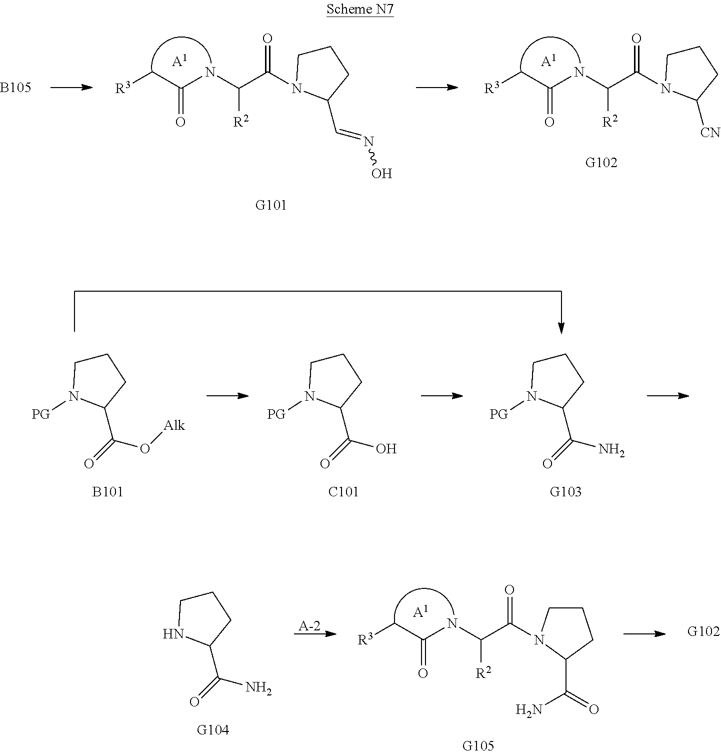

Scheme N7 illustrates a general method to synthesize nitrile compounds of Formula (G102) where a sub-structure of R¹ is depicted and the chemical modifications described here can be applied to other R¹ groups described herein. An aldehyde of Formula (B105) can be condensed with hydroxyamine HCl in an appropriate solvent (e.g., HMPA, DMSO) to afford oxime compound of Formula (G101). Subsequent reaction with Cu(OAc)₂ in HCl/CH₃CN, for example, provides a nitrile of Formula (G102). Alternatively, an aldehyde of Formula (B105) can react with O-(4-(trifluoromethyl)benzoyl)hydroxylamine and L-(−)-camphorsulfonic acid (10%) in methanol to afford a nitrile of Formula (G102) (See An et al., Org. Lett. (2015) 17(20):5064-5067).

An additional method to prepare compounds of Formula (G102) is to start with a protected ester of Formula (B101) and then convert it directly to an amide of Formula (G103) by reaction with ammonia in THF or methanol in a sealed reactor. An ester of Formula (B101) can also be transformed into an amide of Formula (G103) via a two-step process where the ester of Formula (B101) is first hydrolyzed to a carboxylic acid (for example, using LiOH, water and THF when Alk is methyl) to a compound of Formula (C101). Subsequent transformation of the carboxylic acid of Formula (C101) to Formula (G103) can be carried out using a coupling agent (e.g., COMU, EDC) in the presence of ammonia, in an appropriate solvent (such as DMF or CH₃CN). Deprotection of the nitrogen protecting group of Formula (G103), for example, with HCl when PG is Boc, can generate the free amine which can be coupled with a compound of Formula (A-2) (using a readily available amino acid coupling agent) to afford a compound of Formula (G105). Dehydration of the amide group of Formula (G105) using methods described in the literature, for example the Burgess reagent, or trifluoroacetic anhydride (TFAA) can provide the cyano compound of Formula (G102).

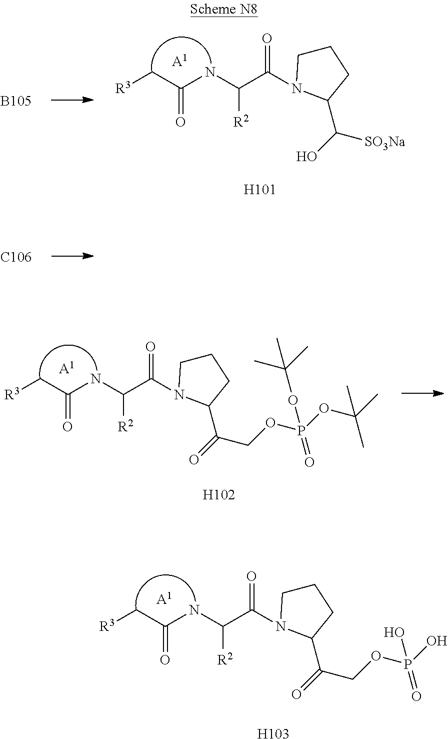

Compounds of Formula (I), or a pharmaceutically acceptable salt thereof, can include a prodrug-type and phosphate-containing moieties at R⁴. An example of a method is depicted in Scheme N8 where a sub-structure of R¹ is depicted and the chemical modifications described here can be applied to other R¹ groups described herein. For example, an aldehyde of Formula (B105) can be transformed into the bisulfite adduct of Formula (H101), by treatment with NaHSO₃. A hydroxyketone of Formula (C106) can be transformed to the phosphate of Formula (H102), for example, by treatment with di-t-butyl N,N-dipropan-2-ylphosphoramidite and tetrazole followed by oxidation with H₂O₂. A compound of Formula (H102) can be deprotected (for example, by treatment with TFA) to provide a compound of Formula (H103).

### Pharmaceutical Compositions

Some embodiments described herein relate to a pharmaceutical composition, which can include an effective amount of a compound described herein (e.g., a compound, or a pharmaceutically acceptable salt thereof, as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the chosen route of administration. Techniques for formulation and administration of the compounds described herein are known to those skilled in the art. Multiple techniques of administering a compound exist in the art including, but not limited to, oral, rectal, topical, aerosol, injection, inhalation and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a coronavirus infection.

Some embodiments disclosed herein relate to a method of treating a coronavirus infection that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a coronavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a coronavirus.

In some embodiments, the coronavirus can be an α-coronavirus or a 3-coronavirus. A compound described herein may be effective against one or more variants of a coronavirus. Examples of variants include, but are not limited, to alpha-variant (B.1.1.7), beta-variant (B.1.351), gamma variant (P.1) and delta-variant (B.1.617.2). In some embodiments, the coronavirus can be selected from CoV 229E, CoV NL63, CoV OC43, CoV HKU1, Middle East Respiratory Syndrome (MERS)—CoV, Severe Acute Respiratory Syndrome (SARS)—CoV, and SARS-CoV-2.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a picornavirus infection.

Some embodiments disclosed herein relate to a method of treating a picornavirus infection that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a picornavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a picornavirus.

In some embodiments, the picornavirus can be a rhinovirus, including rhinovirus A, B and/or C. In some embodiments, a compound described herein, including a compound of Formula (I), or a pharmaceutically acceptable salt thereof, can be used to treat one or serotypes of a rhinovirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a norovirus infection.

Some embodiments disclosed herein relate to a method of treating a norovirus infection that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a norovirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a norovirus.

Some embodiments disclosed herein relate to a method of treating a respiratory condition that is developed because of a coronavirus and/or a picornavirus infection that can include administering to a subject suffering from the respiratory condition and/or contacting a cell infected with the coronavirus and/or the picornavirus in a subject suffering from the respiratory condition with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a respiratory condition due to a coronavirus infection and/or a picornavirus infection with an effective amount of the compound, or a pharmaceutically acceptable salt thereof. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating a respiratory condition due to a coronavirus infection and/or a picornavirus infection.

A subject infected with a coronavirus can be asymptotic. A coronavirus infection can manifest itself via one or more symptoms. Examples of symptoms include, but are not limited to, coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness and/or palpitations. A coronavirus infection can cause complications. A non-limiting list of complications include, but are not limited to, sinusitis, otitis media, pneumonia, acute respiratory distress syndrome, disseminated intravascular coagulation, pericarditis and/or kidney failure.

As with a coronavirus, a subject infected with a picornavirus can be asymptotic. Alternatively, a subject can exhibit one or more of symptoms. Examples of symptoms of a picornavirus infection include, but are not limited to, aseptic meningitis, rash, conjunctivitis, runny nose a headache a cough a fever, a sore throat, chest and/or abdominal pain and paralysis. As provided herein, subjects infected with a norovirus can exhibit one or more the symptoms including, but not limited to, nausea, non-bloody diarrhea, vomiting and abdominal pain. An example of a complication that can be attributed to a norovirus infection is dehydration, including severe dehydration.

Various indicators for determining the effectiveness of a method for treating a coronavirus, picornavirus and/or norovirus infection are also known to those skilled in the art. Examples of suitable indicators include, but are not limited to, a reduction in viral load indicated by reduction in coronavirus (or load) (e.g., reduction<10⁵ copies/mL in serum), a reduction in plasma viral load, a reduction in viral replication, a reduction in time to seroconversion (virus undetectable in patient serum), an increase in the rate of sustained viral response to therapy a reduction of morbidity or mortality in clinical outcomes, reduction in the need for a ventilator and/or total time on a ventilator, reduction in hospitalization rates and/or reduction in time in an ICU (intensive care unit) and/or hospital.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, camels, non-human primates, such as monkeys, chimpanzees, and apes, and, in particular, humans. In some embodiments, the subject can be human, for example, a human subject that is 60 years old or older.

The term “effective amount” is used to indicate an amount of an active compound, or pharmaceutical agent, which elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In some embodiments, the subject can be asymptomatic, for example, the subject can be infected with coronavirus but does not exhibit any symptoms of the viral infection. In some embodiments, the subject can be have a pre-existing condition, such as asthma, hypertension, immunocompromised subjects (such as subjects with cancer, HIV and/or genetic immune deficiencies, bone marrow transplant subjects, solid organ transplant subjects, subjects who have had stem cells for cancer treatment and/or subjects who use oral or intravenous corticosteroids or other medicines called immunosuppressants), liver disease, subjects at risk for severe illness, chronic kidney disease being treated with dialysis, chronic lung disease, diabetes, hemoglobin disorders, serious heart conditions (for example, heart failure, coronary artery disease, congenital heart disease, cardiomyopathies, and pulmonary hypertension), severe obesity (such as subjects with a body mass index (BMI) of 40 or above) and people who live in a nursing home or long-term care facility. Additional examples and/or further information is provided by the CDC (https://www.cdc.gov/coronavirus/2019-ncov/need-extra-precautions/groups-at-higher-risk.html).

A compound described herein, including a compound of Formula (I), or a pharmaceutically acceptable salt thereof, can be administered after a subject is infected with a coronavirus. In addition and/or alternative, a compound described herein, including a compound of Formula (I), or a pharmaceutically acceptable salt thereof, can be administered prophylactically.

Examples of agents that have been used to treat a coronavirus infection include Remdesivir. However, there can be drawbacks associated with compounds being used to treat a coronavirus including, but not limited to, one or more adverse side effects, the need for subcutaneous administration and/or high cost. Potential advantages of a compound of Formula (I), or a pharmaceutically acceptable salt thereof, can be less adverse side effects, delay in the onset of an adverse side effect and/or reduction in the severity of an adverse side effect.

A coronavirus infection can be treated by inhibiting certain mechanisms. In some embodiments, a compound described herein (such as a compound of Formula (I), or a pharmaceutically acceptable salt thereof) can be selective for a coronavirus protease. For example, a compound of Formula (I), or a pharmaceutically acceptable salt thereof, can be selective for a coronavirus protease compared to a host protease, for example, one or more host proteases selected from Cathepsin L, Cathepsin B, Cathepsin D, Cathepsin K, Leukocyte Elastase, Chymotrypsin, Trypsin, Thrombin, Pepsin, Caspase 2, Elastase and Calpain. In some embodiments, the selectivity for a coronavirus protease over a host protease (such as those described herein) can be >2-fold. In some embodiments, the selectivity for a coronavirus protease over a host protease (such as those described herein) can be >10-fold. In some embodiments, the selectivity for a coronavirus protease over a host protease (such as those described herein) can be >100-fold.

Studies have shown that the entry of SARS-CoV-2 into the target cells is a process that can be mediated by multiple proteases including cysteine cathepsins L and/or transmembrane protease serine 2 (TMPRSS2) (Shang et al., PNAS (2020) 117:11727, and Hoffmann et al., Cell (2020) 181:271-280). The cathepsin L inhibitor K117777, which lacks an inhibitory effect on the 3Clpro, can result in potent inhibition of SARS-CoV-2 in VeroE6, A549-ACE2 and/or HeLa-ACE2 (Mellott et al., bioRxiv (2020) 2020.2010.2023.347534). It has also been shown that the potent antiviral effect of K117777 is abolished when TMPRSS2 was expressed In A549-ACE2 (Steuten et al., bioRxiv (2020) 2020.2011.2021.392753). Off target activity of 3cLpro inhibitors, for example, on cathepsin L, may lead to an inaccurate assessment of the 3cLpro component of a compound's cellular potency. As an example, a compound described herein (such as a compound of Formula (I), or a pharmaceutically acceptable salt thereof) can have greater selectivity for a coronavirus protease over a host protease, such as cathepsin L. The selectivity can be determined by those skilled in the art, for example, using IC₅₀ and/or Ki values. In some embodiments, a compound described herein does not significantly inhibit cathepsin L (for example, IC₅₀≥10000 nM or >3.3 μM), but inhibits a coronavirus protease (for example, SARS-Cov-2 3Clpro).

A drawback with anti-viral treatment can be the development of resistance, including cross-resistance. Resistance can be a cause for treatment failure. The term “resistance” as used herein refers to a viral strain displaying a delayed, lessened and/or null response to an anti-viral agent. In some embodiments, a compound, or a pharmaceutically acceptable salt thereof, as described herein can be provided to a subject infected with a coronavirus strain that is resistant to one or more other anti-viral agents. In some embodiments, development of coronavirus resistant strains is delayed when a subject is treated with a compound, or a pharmaceutically acceptable salt thereof, as described herein compared to the development of a coronavirus resistant strain when treated with one or more other anti-viral agents.

### Combination Therapies

In some embodiments, a compound, or a pharmaceutically acceptable salt thereof, as described herein can be used in combination with one or more additional agent(s) for treating and/or inhibiting replication a coronavirus. Additional agents include, but are not limited to, an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a monoclonal antibody, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine. Examples of additional agents include Ascorbic acid, Anakin, Azithromycin, Baloxavir, Baricitinib, Chloroquine Phosphate, Colchicine, a corticosteroid, Epoprostenol, Famotidine, Favipiravir, an IGIV, an interferon (for example, recombinant interferon alpha 2b, IFN-α and/or PEG-IFN-α-2a), an IVIG, Ivermectin, γ-globulin, lopinavir, Methylprednisolone, Molnupiravir (MK-4482 or EIDD-2801), Niclosamide, Nitazoxanide, Nitric oxide, Oseltamivir, Peramivir, RANTES, ribavirin, Remdesivir, Ruxolitinib, Sarilumab, Siltuximab, Sirolimus, a statin, Tacrolimus, Tocilizumab, Umifenovir, Zanamivir, Casirivimab, imdevimab, bamlanivimab, etesevimab, GS-5245 (Obeldesivir) and AT-527 (Good et al., Antimicrobial Agents and Chemotherapy (2021) 65(4):e02479-20)

In some embodiments, a compound, or a pharmaceutically acceptable salt thereof, as described herein can be administered with one or more additional agent(s) together in a single pharmaceutical composition. In some embodiments, a compound, or a pharmaceutically acceptable salt thereof, can be administered with one or more additional agent(s) as two or more separate pharmaceutical compositions. Further, the order of administration of a compound, or a pharmaceutically acceptable salt thereof, as described herein with one or more additional agent(s) can vary.

### EXAMPLES

Additional embodiments are disclosed in further detail in the following examples, which are not in any way intended to limit the scope of the claims.

### Compounds

Compounds of Formula (I), along with pharmaceutically acceptable salts thereof, can be prepared in various ways, including those synthetic schemes shown and described herein, are provided below. Those skilled in the art will be able to recognize modifications of the disclosed syntheses and to devise routes based on the disclosures herein; all such modifications and alternate routes are within the scope of the claims.

### Synthesis of Intermediates

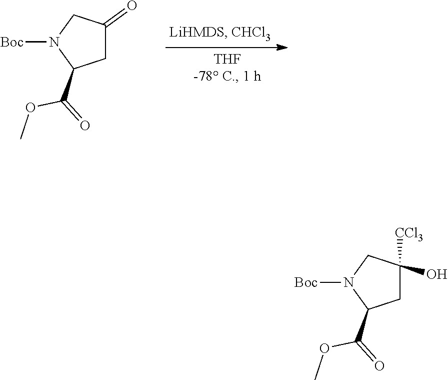

To a solution of 1-(t-butyl) 2-methyl (S)-4-oxopyrrolidine-1,2-dicarboxylate (10.0 g×3, 123 mmol) and chloroform (9.8 g×3, 247 mmol) in THF (300 mL) was added lithium bis(trimethylsilyl)amide (82 mL×3, 246 mmol, 1M in THF) stirred at −78° C. under nitrogen. The mixture was stirred for 1 h at −78° C. and the reaction was quenched with sat. NH₄Cl aqueous solution (3×150 mL). The mixture was extracted with EtOAc (3×700 mL). The organic layers were combined, washed with brine (2×300 mL) and dried over anhydrous sodium sulfate. The solids were removed by filtration and the filtrate was concentrated under reduced pressure. The crude product was chromatographed on a silica gel column with ethyl acetate (EA):petroleum ether (PE) (30-50%) to provide 1-(t-butyl) 2-methyl (2S,4S)-4-hydroxy-4-(trichloromethyl)pyrrolidine-1,2-dicarboxylate (13 g, crude) as a light yellow solid. ¹H NMR (400 MHz, DMSO-d₆) δ 6.84-6.86 (m, 1H), 4.50-4.71 (m, 1H), 3.74-3.91 (m, 1H), 3.58-3.73 (m, 4H), 2.81-3.16 (m, 1H), 2.51-2.60 (m, 0.3H), 2.13-2.25 (m, 0.63H), 1.30-1.46 (m, 9H). LC-MS (ESI, m/z): 306 [M-56+H]⁺.

### t-butyl (2R,5′S)-5′-carbamoyl-5,7-difluoro-3-oxo-3,4-dihydrospiro[benzo[b][1,4]oxazine-2,3′-pyrrolidine]-1′-carboxylate

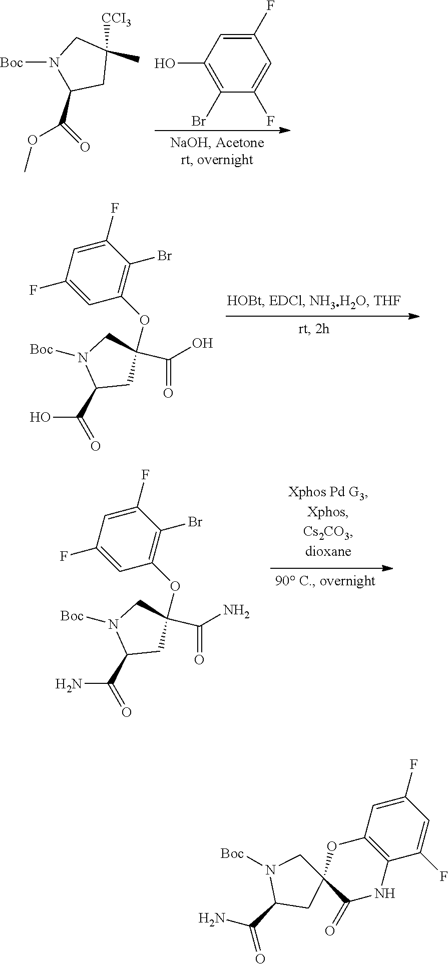

To a mixture of 1-t-butyl 2-methyl (2S,4S)-4-hydroxy-4-(trichloromethyl)pyrrolidine-1,2-dicarboxylate (12.0 g, 33.1 mmol) and 2-bromo-3,5-difluorophenol (13.8 g, 66.2 mmol) in acetone (150 mL) was added NaOH (8.00 g, 199 mmol) at 0° C. The mixture was stirred overnight at rt and the reaction was quenched with water (150 mL). The mixture was adjusted to pH=5 with HCl (1 M) and then extracted with EA (3×150 mL). The organic layers were combined, washed with brine (2×100 mL) and dried over anhydrous sodium sulfate. The solids were removed by filtration and the filtrate was concentrated under reduced pressure to remove the solvent. The crude product was purified by C18 column with CH₃CN/Water (0.05% TFA), and the fraction was concentrated under reduced pressure to afford (2S,4R)-4-(2-bromo-3,5-difluorophenoxy)-1-(t-butoxycarbonyl)pyrrolidine-2,4-dicarboxylic acid (7.0 g, crude) as a brown oil. LC-MS (ESI, m/z): 410 [M-56+H]⁺.

To a mixture of (2S,4R)-4-(2-bromo-3,5-difluorophenoxy)-1-(t-butoxycarbonyl)pyrrolidine-2,4-dicarboxylic acid (7.00 g, 15.0 mmol) in THF (80 mL) were added 1-hydroxybenzotriazole (10.0 g, 75.1 mmol) and 1-(3-dimethylaminopropyl)-3-ethylcarbodiimide hydrochloride (11.5 g, 60.0 mmol) at 0° C. After stirring for 30 mins at room temperature (rt), ammonium hydroxide (80 mL, 30% in water) was added at 0° C. The mixture was stirred for 2 h at rt and then diluted with water (100 mL). The mixture was extracted with EA (3×100 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with MeOH:DCM (8:92) to provide t-butyl (2S,4R)-4-(2-bromo-3,5-difluorophenoxy)-2,4-dicarbamoylpyrrolidine-1-carboxylate (1.2 g, 16%) as a light yellow solid. ¹H NMR (400 MHz, DMSO-d₆) δ 8.00-8.10 (m, 1H), 7.76 (s, 1H), 7.40-7.52 (m, 1H), 7.19-7.31 (m, 1H), 6.98-7.09 (m, 1H), 6.40-6.60 (m, 1H), 4.08-4.25 (m, 1H), 3.95-4.06 (m, 1H), 3.64-3.72 (m, 1H), 2.60-2.72 (m, 1H), 2.24-2.36 (m, 1H), 1.31-1.38 (m, 9H). LC-MS (ESI, m/z): 408 [M−56+H]⁺.

To a mixture of t-butyl (2S,4R)-4-(2-bromo-3,5-difluorophenoxy)-2,4-dicarbamoylpyrrolidine-1-carboxylate (1.2 g, 2.58 mmol), methanesulfonato(2-dicyclohexylphosphino-2′,4′,6′-tri-1-propyl-1,1′-biphenyl)(2′-amino-1,1′-biphenyl-2-yl)palladium(II) (219 mg, 0.259 mmol) and 2-(dicyclohexylphosphino)-2′,4′,6′-triisopropylbiphenyl (123 mg, 0.259 mmol) in dioxane (15 mL) was added cesium carbonate (1.80 g, 5.62 mmol). The mixture was stirred overnight at 90° C. under nitrogen and the reaction was quenched with water (50 mL). The mixture was extracted with EA (3×50 mL). The organic layers were combined, washed with brine (2×50 mL) and dried over anhydrous sodium sulfate. The solids were removed by filtration and the filtrate was concentrated under reduced pressure. The crude product was chromatographed on a silica gel column with MeOH:DCM (1:25) to provide t-butyl (2R,5′S)-5′-carbamoyl-5,7-difluoro-3-oxo-4H-spiro[1,4-benzoxazine-2,3′-pyrrolidine]-1′-carboxylate (670 mg, 64%) as a light yellow solid.

¹H NMR (400 MHz, DMSO-d₆) δ 11.20 (s, 1H), 7.49-7.60 (m, 1H), 6.99-7.18 (m, 2H), 6.80-6.94 (m, 1H), 4.17-4.32 (m, 1H), 3.64-3.81 (m, 2H), 2.50-2.55 (m, 1H), 2.22-2.36 (m, 1H), 1.37 (s, 9H). LC-MS (ESI, m/z): 328 [M−56+H]⁺.

### (2R,5′S)-5,7-difluoro-3-oxo-3,4-dihydrospiro[benzo[b][1,4]oxazine-2,3′-pyrrolidine]-5′-carboxamide

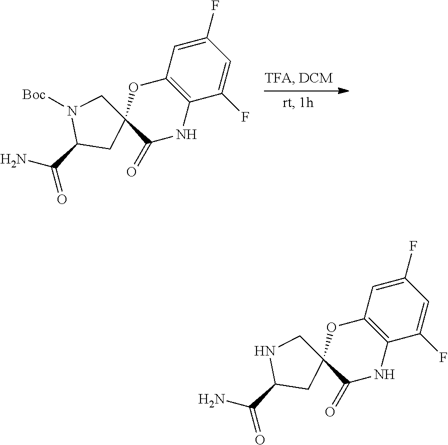 [SMILES: CC(C)(C)OC(=O)N1C[C@@]2(C[C@H]1C(N)=O)Oc1cc(F)cc(F)c1NC2=O ; NC(=O)[C@@H]1C[C@]2(CN1)Oc1cc(F)cc(F)c1NC2=O]

To a solution of t-butyl (2R,5′S)-5′-carbamoyl-5,7-difluoro-3-oxo-3,4-dihydrospiro[benzo[b][1,4]oxazine-2,3′-pyrrolidine]-1′-carboxylate (100 mg, 0.261 mmol) in DCM (3 mL) was added TFA (1 mL). The mixture was stirred for 1 h at rt and concentrated under reduced pressure to afford (2R,5′S)-5,7-difluoro-3-oxo-3,4-dihydrospiro[benzo[b][1,4]oxazine-2,3′-pyrrolidine]-5′-carboxamide (75 mg, crude) as a yellow solid. LC-MS (ESI, m/z): 306 [M+Na]⁺.

### t-butyl (2R,5′S)-5′-carbamoyl-3-oxo-3,4-dihydrospiro[pyrido[3,2-b][1,4]oxazine-2,3′-pyrrolidine]-1′-carboxylate

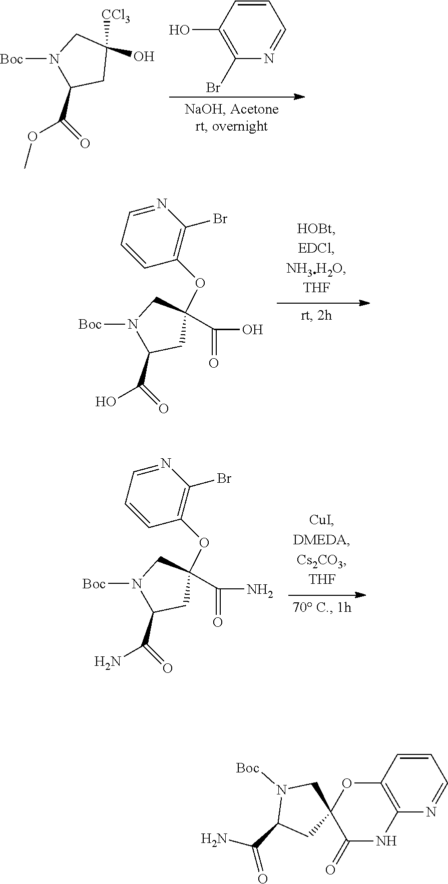

To a mixture of 1-(t-butyl) 2-methyl (2S,4S)-4-hydroxy-4-(trichloromethyl)pyrrolidine-1,2-dicarboxylate (5.00 g, 13.7 mmol), 2-bromopyridin-3-ol (4.80 g, 27.5 mmol) in acetone (60 mL) was added NaOH (3.31 g, 82.7 mmol) at 0° C. The mixture was stirred overnight at rt and adjusted to pH=6 with HCl (4 M in dioxane) at 0° C. The mixture was concentrated under reduced pressure to remove the solvent. The crude product was purified by C18 column with CH₃CN/Water (0.05% TFA), and the fraction was concentrated under reduced pressure to afford (2S,4R)-4-((2-bromopyridin-3-yl)oxy)-1-(t-butoxycarbonyl)pyrrolidine-2,4-dicarboxylic acid (4.40 g, crude) as a red oil. LC-MS (ESI, m/z): 375 [M−56+H]⁺.

To a mixture of (2S,4R)-4-((2-bromopyridin-3-yl)oxy)-1-(t-butoxycarbonyl)pyrrolidine-2,4-dicarboxylic acid (4.40 g, 10.2 mmol) in THF (50 mL) were added hydroxybenzotriazole (8.27 g, 61.2 mmol) and 1-(3-dimethylaminopropyl)-3-ethylcarbodiimide hydrochloride (9.78 g, 51.0 mmol) at 0° C. After stirring for 30 min at rt, ammonium hydroxide (60 mL, 30% in water) was added at 0° C. The mixture was stirred for 2 h at rt and diluted with water (100 mL). The mixture was extracted with EA (3×150 mL). The organic layers were combined, washed with brine (150 mL) and dried over anhydrous sodium sulfate. The solids were removed by filtration and the filtrate was concentrated under reduced pressure. The crude product was purified by C18 column with CH₃CN/Water (0.05% NH₄HCO₃), and the fraction was concentrated under reduced pressure to provide t-butyl (2S,4R)-4-((2-bromopyridin-3-yl)oxy)-2,4-dicarbamoylpyrrolidine-1-carboxylate (1.00 g, 22%) as a red solid. ¹H NMR (400 MHz, DMSO-d₆) δ 8.00-8.10 (m, 2H), 7.70-7.78 (m, 1H), 7.39-7.55 (m, 2H), 7.15-7.23 (m, 1H), 6.99-7.07 (m, 1H), 4.08-4.29 (m, 1H), 3.92-4.04 (m, 1H), 3.65-3.78 (m, 1H), 2.59-2.67 (m, 1H), 2.25-2.43 (m, 1H), 1.29-1.50 (m, 6H), 1.24-1.27 (m, 3H). LC-MS (ESI, m/z): 373 [M−56+H]⁺.

To a mixture of t-butyl (2S,4R)-4-((2-bromopyridin-3-yl)oxy)-2,4-dicarbamoylpyrrolidine-1-carboxylate (1.13 g, 2.63 mmol), copper(I) iodide (300 mg, 1.57 mmol) and cesium carbonate (1.72 g, 5.26 mmol) in THF (15 mL) was added N,N′-Dimethyl-1,2-ethanediamine (394 mg, 4.47 mmol). The mixture was stirred for 1 h at 70° C. under nitrogen and the reaction was quenched with water (50 mL). The mixture was extracted with EA (3×50 mL). The organic layers were combined, washed with brine (2×50 mL) and dried over anhydrous sodium sulfate. The solids were removed by filtration and the filtrate was concentrated under reduced pressure. The crude product was purified by C18 column with CH₃CN/Water (0.05% TFA), and the fraction was concentrated under reduced pressure to provide t-butyl (2R,5′S)-5′-carbamoyl-3-oxo-3,4-dihydrospiro[pyrido[3,2-b][1,4]oxazine-2,3′-pyrrolidine]-1′-carboxylate (590 mg, 64%) as a light yellow solid. ¹H NMR (400 MHz, DMSO-d₆) δ 11.53 (s, 1H), 7.93-8.06 (m, 1H), 7.34-7.58 (m, 2H), 6.94-7.20 (m, 2H), 4.17-4.41 (m, 1H), 3.62-3.80 (m, 2H), 2.43-2.48 (m, 1H), 2.25-2.37 (m, 1H), 1.31-1.49 (m, 9H). LC-MS (ESI, m/z):293[M−56+H]⁺.

### (2R,5′S)-3-oxo-3,4-dihydrospiro[pyrido[3,2-b][1,4]oxazine-2,3′-pyrrolidine]-5′-carboxamide

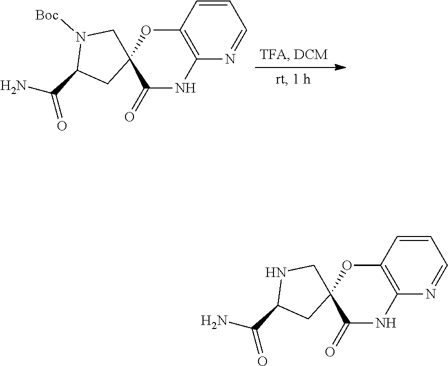 [SMILES: CC(C)(C)OC(=O)N1C[C@@]2(C[C@H]1C(N)=O)Oc1cccnc1NC2=O ; NC(=O)[C@@H]1C[C@]2(CN1)Oc1cccnc1NC2=O]

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (300 mg, 0.861 mmol) in DCM (12 mL) was added TFA (4 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (210 mg, crude) as a yellow solid. LC-MS (ESI, m/z): 249 [M+H]⁺.

### t-butyl (5′S)-5′-carbamoyl-3-oxo-3,4-dihydrospiro[benzo[b][1,4]oxazine-2,3′-pyrrolidine]-1′-carboxylate

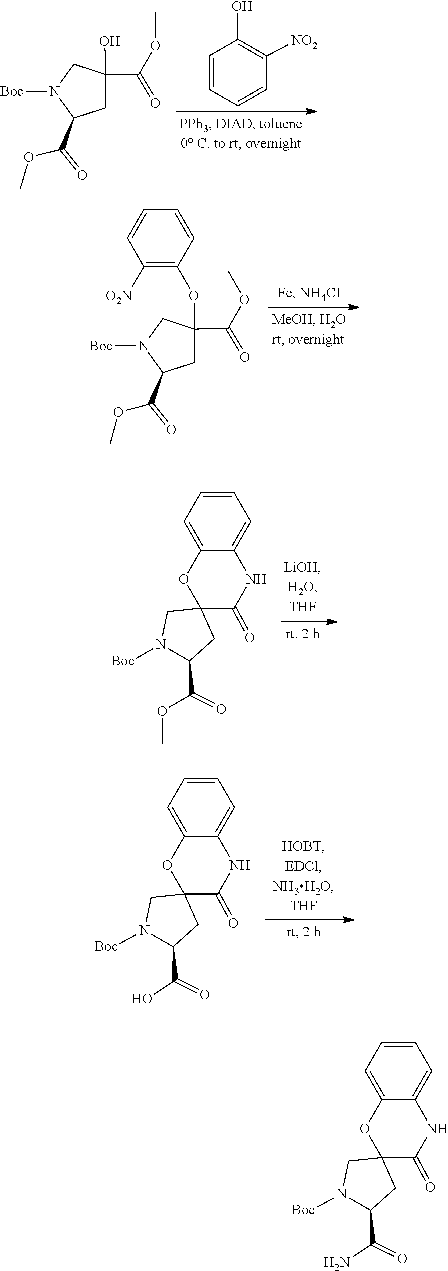

To a mixture of 1-(t-butyl) 2,4-dimethyl (2S)-4-hydroxypyrrolidine-1,2,4-tricarboxylate (800 mg, 2.64 mmol), o-nitrophenol (367 mg, 2.64 mmol) in toluene (8 mL) was added triphenylphosphane (997 mg, 3.17 mmol) at 0° C. under nitrogen. The mixture was stirred for 20 min at 0° C. Diisopropyl azodicarboxylate (768 mg, 3.17 mmol) was then added at 0° C. for 20 min. The mixture was stirred for overnight at rt. The reaction was quenched with water (30 mL). The mixture was extracted with EA (3×80 mL). The organic layers were combined, washed with brine (2×40 mL) and dried over anhydrous Na₂SO₄. The solids were removed by filtration and the filtrate was concentrated under reduced pressure to afford the crude product that was chromatographed on a silica gel column with EA:PE (1:1) to provide 1-(t-butyl) 2,4-dimethyl (2S)-4-(2-nitrophenoxy)pyrrolidine-1,2,4-tricarboxylate (2.00 g, crude) as a red oil. LC-MS (ESI, m/z): 425 [M+H]⁺.

To a mixture of 1-(t-butyl) 2,4-dimethyl (2S)-4-(2-nitrophenoxy)pyrrolidine-1,2,4-tricarboxylate (1.12 g, 2.64 mmol) and NH₄Cl (340 mg, 6.34 mmol) in MeOH (12 mL) and water (3 mL) was added iron (738 mg, 5.47 mmol) at rt. The mixture was stirred overnight, and then filtered through a celite pad and washed with DCM (3×50 mL). The organic layers were concentrated under reduced pressure to afford 1′-(t-butyl) 5′-methyl (5′S)-3-oxo-3,4-dihydrospiro[benzo[b][1,4]oxazine-2,3′-pyrrolidine]-1′,5′-dicarboxylate (2.81 g, crude) as a red oil. LC-MS (ESI, m/z): 363 [M+H]⁺.

To a stirred of 1′-(t-butyl) 5′-methyl (5′S)-3-oxo-3,4-dihydrospiro[benzo[b][1,4]oxazine-2,3′-pyrrolidine]-1′,5′-dicarboxylate (956 mg, 2.64 mmol) in THF (9 mL) and water (9 mL) was added LiOH (317 mg, 13.2 mmol) at rt. The mixture was stirred for 2 h and acidified to pH=3 with HCl (2M). The aqueous layer was extracted with EA (3×100 mL), dried over anhydrous Na₂SO₄ and concentrated under reduced pressure to afford (5′S)-1′-(t-butoxycarbonyl)-3-oxo-3,4-dihydrospiro[benzo[b][1,4]oxazine-2,3′-pyrrolidine]-5′-carboxylic acid (730 mg, 76%) as a yellow oil. LC-MS (ESI, m/z): 349 [M+H]⁺.

To a mixture of (5′S)-1′-(t-butoxycarbonyl)-3-oxo-3,4-dihydrospiro[benzo[b][1,4]oxazine-2,3′-pyrrolidine]-5′-carboxylic acid (730 mg, 2.09 mmol) in THF (7 mL) were added 1-hydroxybenzotriazole (847 mg, 6.27 mmol) and 1-(3-dimethylaminopropyl)-3-ethylcarbodiimide hydrochloride (878 mg, 4.60 mmol). The mixture was stirred for 30 min at 0° C. Ammonia (14.6 mL) was added at 0° C. and the mixture was stirred for 2 h at rt. The mixture was purified by C18 column with CH₃CN/water (0.05% NH₄HCO₃). The fraction was concentrated under reduced pressure to provide t-butyl (5′S)-5′-carbamoyl-3-oxo-3,4-dihydrospiro[benzo[b][1,4]oxazine-2,3′-pyrrolidine]-1′-carboxylate (730 mg, crude) as a yellow solid. LC-MS (ESI, m/z): 348 [M+H]⁺.

### (2R,5′S)-3-oxo-3,4-dihydrospiro[benzo[b][1,4]oxazine-2,3′-pyrrolidine]-5′-carboxamide

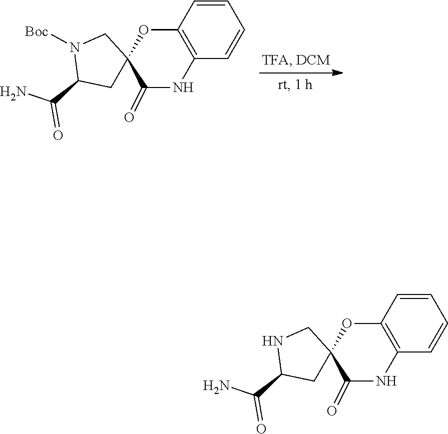 [SMILES: CC(C)(C)OC(=O)N1C[C@@]2(C[C@H]1C(N)=O)Oc1ccccc1NC2=O ; NC(=O)[C@@H]1C[C@]2(CN1)Oc1ccccc1NC2=O]

To a mixture of t-butyl (2R,5′S)-5′-carbamoyl-3-oxo-3,4-dihydrospiro[benzo[b][1,4]oxazine-2,3′-pyrrolidine]-1′-carboxylate (100 mg, 0.288 mmol) in DCM (3 mL) was added TFA (1 mL). The mixture was stirred for 1 h at rt and concentrated under reduced pressure to afford (2R,5′S)-3-oxo-3,4-dihydrospiro[benzo[b][1,4]oxazine-2,3′-pyrrolidine]-5′-carboxamide (68.0 mg, crude) as a yellow oil. LC-MS (ESI, m/z): 270 [M+Na]⁺.

### t-butyl (3R,5′S)-5′-carbamoyl-2-oxo-1,2-dihydrospiro[pyrido[2,3-b][1,4]oxazine-3,3′-pyrrolidine]-1′-carboxylate

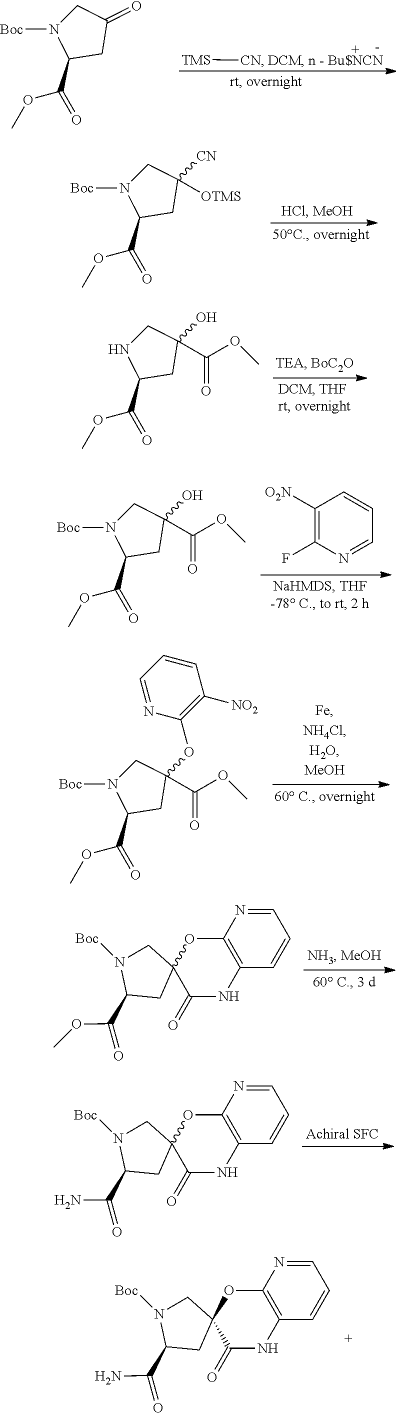

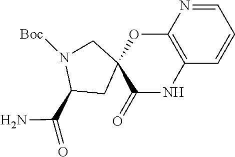 [SMILES: CC(C)(C)OC(=O)N1C[C@@]2(C[C@H]1C(N)=O)Oc1ncccc1NC2=O]

To a mixture of 1-(t-butyl) 2-methyl (S)-4-oxopyrrolidine-1,2-dicarboxylate (10.0 g, 41.1 mmol) in DCM (100 mL) were added trimethylsilyl cyanide (8.20 g, 82.2 mmol) and tetrabutylammonium cyanide (1.20 g, 4.11 mmol). The mixture was stirred overnight on the magnetic stirrer at rt under nitrogen and the reaction was quenched with water (150 mL). The mixture was extracted with DCM (3×100 mL). The organic layers were combined, washed with brine (2×100 mL) and dried over anhydrous sodium sulfate. The solids were removed by filtration and the filtrate was concentrated under reduced pressure to afford 1-(t-butyl) 2-methyl (2S)-4-cyano-4-((trimethylsilyl)oxy)pyrrolidine-1,2-dicarboxylate (14.0 g, crude) as a brown oil. LC-MS (ESI, m/z): 287 [M−56+H]⁺.

A mixture of 1-(t-butyl) 2-methyl (2S)-4-cyano-4-((trimethylsilyl)oxy)pyrrolidine-1,2-dicarboxylate (14.0 g, 41.1 mmol) in hydrogen chloride (140 mL, 4 M in MeOH) was stirred overnight at 50° C. The mixture was concentrated under reduced pressure to afford dimethyl (2S)-4-hydroxypyrrolidine-2,4-dicarboxylate (8.40 g, crude) as a black oil. LC-MS (ESI, m/z): 204 [M+H]⁺.

To a mixture of dimethyl (2S)-4-hydroxypyrrolidine-2,4-dicarboxylate (8.4.0 g, 41.3 mmol) in DCM (85 mL)/THF (35 mL) were added trimethylamine (17.0 g, 165 mmol) and di-t-butyl dicarbonate (18.0 g, 82.6 mmol). The mixture was stirred overnight at rt and the reaction was quenched with water (100 mL). The mixture was extracted with EA (3×100 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with EA:PE (1:4) to provide 1-(t-butyl) 2,4-dimethyl (2S)-4-hydroxypyrrolidine-1,2,4-tricarboxylate (6.90 g, 49% over 3 steps, R:S=4:6) as a yellow oil. ¹H NMR (400 MHz, DMSO-d₆) δ 5.73-6.13 (m, 1H), 4.20-4.46 (m, 1H), 3.35-3.75 (m, 8H), 2.55-2.68 (m, 0.6H), 2.07-2.38 (m, 1.4H), 1.28-1.46 (m, 9H). LC-MS (ESI, m/z): 204 [M-boc+H]⁺.

To a mixture of 1-(t-butyl) 2,4-dimethyl (2S)-4-hydroxypyrrolidine-1,2,4-tricarboxylate (5.00 g, 16.5 mmol) in THF (150 mL) was added dropwise sodium bis(trimethylsilyl)amide (12.4 mL, 24.8 mmol, 2 M in THF) at −78° C. under nitrogen. After stirring for 5 min at the same temperature, a solution of 2-fluoro-3-nitropyridine (3.51 g, 24.8 mmol) in THF (10 mL) was added dropwise at −78° C. The mixture was stirred for 2 h from −78° C. to rt under nitrogen and the reaction was quenched with sat. aqueous ammonium chloride (300 mL). The mixture was extracted with EA (3×300 mL). The organic layers were combined, washed with brine (2×300 mL) and dried over anhydrous sodium sulfate. The solids were removed by filtration and the filtrate was concentrated under reduced pressure. The crude product was chromatographed on a silica gel column with EA:PE (21:79) to provide 1-(t-butyl) 2,4-dimethyl (2S)-4-((3-nitropyridin-2-yl)oxy)pyrrolidine-1,2,4-tricarboxylate (5.50 g, 58%) as a light yellow solid. ¹H NMR (400 MHz, DMSO-d₆) δ 8.40-8.56 (m, 2H), 7.30-7.38 (m, 1H), 4.16-4.65 (m, 2H), 3.86-4.09 (m, 1H), 3.62-3.72 (m, 6H), 2.81-3.02 (m, 1H), 2.53-2.72 (m, 1H), 1.32-1.41 (m, 9H). LC-MS (ESI, m/z): 448 [M+Na]⁺.

A mixture of 1-(t-butyl) 2,4-dimethyl (2S)-4-((3-nitropyridin-2-yl)oxy)pyrrolidine-1,2,4-tricarboxylate (5.50 g, 12.9 mmol), iron (3.61 g, 64.6 mmol) and ammonium chloride (1.66 g, 31.0 mmol) in MeOH (60 mL)/H₂O (15 mL) was stirred overnight at 60° C. The mixture was filtered through a celite pad and washed with EA (3×50 mL). The filtrate was diluted with water (150 mL). The mixture was extracted with EA (3×150 mL). The organic layers were combined, washed with brine (2×150 mL) and dried over anhydrous sodium sulfate. The solids were removed by filtration and the filtrate was concentrated under reduced pressure. The crude product was chromatographed on a silica gel column with EA:PE (3:7) to provide 1′-(t-butyl) 5′-methyl (5′S)-2-oxo-1,2-dihydrospiro[pyrido[2,3-b][1,4]oxazine-3,3′-pyrrolidine]-1′,5′-dicarboxylate (4.40 g, 84%) as a light yellow solid. ¹H NMR (400 MHz, DMSO-d₆) δ 11.01-11.25 (m, 1H), 7.80-7.95 (m, 1H), 7.23-7.35 (m, 1H), 7.03-7.17 (m, 1H), 4.33-4.62 (m, 1H), 3.91-4.10 (m, 1H), 3.67-3.72 (m, 3H), 3.58-3.65 (m, 1H), 2.63-2.84 (m, 1H), 2.20-2.37 (m, 1H), 1.33-1.42 (m, 9H). LC-MS (ESI, m/z): 386 [M+Na]⁺.

A mixture of 1′-(t-butyl) 5′-methyl (5′S)-2-oxo-1,2-dihydrospiro[pyrido[2,3-b][1,4]oxazine-3,3′-pyrrolidine]-1′,5′-dicarboxylate (4.40 g, 12.1 mmol) in ammonia (100 mL, 7.0 M in MeOH) was stirred for 3 days at 60° C. The mixture was concentrated under reduced pressure. The crude product was chromatographed on a silica gel column with MeOH:DCM (5:95) to provide t-butyl (5′S)-5′-carbamoyl-2-oxo-1,2-dihydrospiro[pyrido[2,3-b][1,4]oxazine-3,3′-pyrrolidine]-1′-carboxylate (3.50 g, 66%) as a yellow solid. ¹H NMR (400 MHz, DMSO-d₆) δ 11.03 (br, 1H), 7.81-7.94 (m, 1H), 7.23-7.36 (m, 2H), 7.01-7.15 (m, 2H), 4.19-4.37 (m, 1H), 3.67-3.79 (m, 1H), 3.50-3.65 (m, 1H), 2.65-2.83 (m, 1H), 2.10-2.28 (m, 1H), 1.33-1.40 (m, 9H). LC-MS (ESI, m/z): 349 [M+H]⁺.

The crude product t-butyl (5′S)-5′-carbamoyl-2-oxo-1,2-dihydrospiro[pyrido[2,3-b][1,4]oxazine-3,3′-pyrrolidine]-1′-carboxylate (3.50 g, 10.0 mmol) was separated by prep-ACIRAL-SFC-HPLC column (Column: GreenSep Basic 3×15 cm, 5 m; Mobile Phase A: CO₂, Mobile Phase B: MeOH(1% 2M NH₃ in MeOH); Flow rate: 75 mL/min; Gradient: isocratic 20% B; Column Temperature (° C.): 35; Back Pressure(bar): 100; Wave Length: 220 nm; RT1(min): 5.13; RT2(min): 6.82; Sample Solvent: MeOH; Injection Volume: 4 mL). Purification resulted in isomer 1: t-butyl (3S,5′S)-5′-carbamoyl-2-oxo-1,2-dihydrospiro[pyrido[2,3-b][1,4]oxazine-3,3′-pyrrolidine]-1′-carboxylate (950 mg, 25%) as a light yellow solid. ¹H NMR (400 MHz, DMSO-d₆) δ 11.06 (s, 1H), 7.79-7.88 (m, 1H), 7.24-7.45 (m, 2H), 7.00-7.19 (m, 2H), 4.20-4.32 (m, 1H), 3.95-4.02 (m, 1H), 3.49-3.65 (m, 1H), 2.63-2.81 (m, 1H), 2.10-2.26 (m, 1H), 1.23-1.51 (m, 9H). LC-MS (ESI, m/z): 349 [M+H]⁺; and Isomer 2: t-butyl (3R,5′S)-5′-carbamoyl-2-oxo-1,2-dihydrospiro[pyrido[2,3-b][1,4]oxazine-3,3′-pyrrolidine]-1′-carboxylate (980 mg, 26%) as a light yellow solid. ¹H NMR (400 MHz, DMSO-d₆) δ 11.12 (s, 1H), 7.79-7.95 (m, 1H), 7.45-7.61 (m, 1H), 7.23-7.39 (m, 1H), 6.93-7.20 (m, 2H), 4.18-4.39 (m, 1H), 3.61-3.80 (m, 2H), 2.43-2.49 (m, 1H), 2.24-2.41 (m, 1H), 1.17-1.51 (m, 9H). LC-MS (ESI, m/z): 349 [M+H]⁺.

### (3R,5′S)-2-oxo-1,2-dihydrospiro[pyrido[2,3-b][1,4]oxazine-3,3′-pyrrolidine]-5′-carboxamide

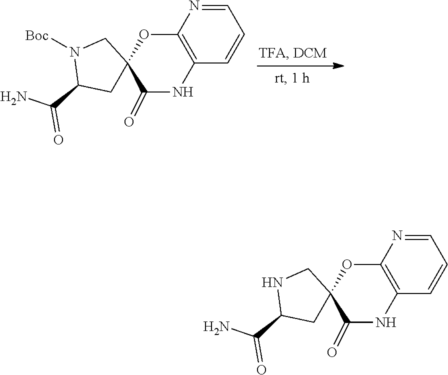 [SMILES: CC(C)(C)OC(=O)N1C[C@@]2(C[C@H]1C(N)=O)Oc1ncccc1NC2=O ; NC(=O)[C@@H]1C[C@]2(CN1)Oc1ncccc1NC2=O]

To a mixture of t-butyl (3R,5′S)-5′-carbamoyl-2-oxo-1,2-dihydrospiro[pyrido[2,3-b][1,4]oxazine-3,3′-pyrrolidine]-1′-carboxylate (100 mg, 0.287 mmol) in DCM (3 mL) was added TFA (1 mL). The mixture was stirred for 1 h at rt and concentrated under reduced pressure to afford (3R,5′S)-2-oxo-1,2-dihydrospiro[pyrido[2,3-b][1,4]oxazine-3,3′-pyrrolidine]-5′-carboxamide (70.0 mg crude) as a yellow semi-solid. LC-MS (ESI, m/z): 249 [M+H]⁺.

### t-butyl (3S,5S)-5-carbamoyl-2′-oxo-1′,4′-dihydro-2′H-spiro[pyrrolidine-3,3′-quinoline]-1-carboxylate

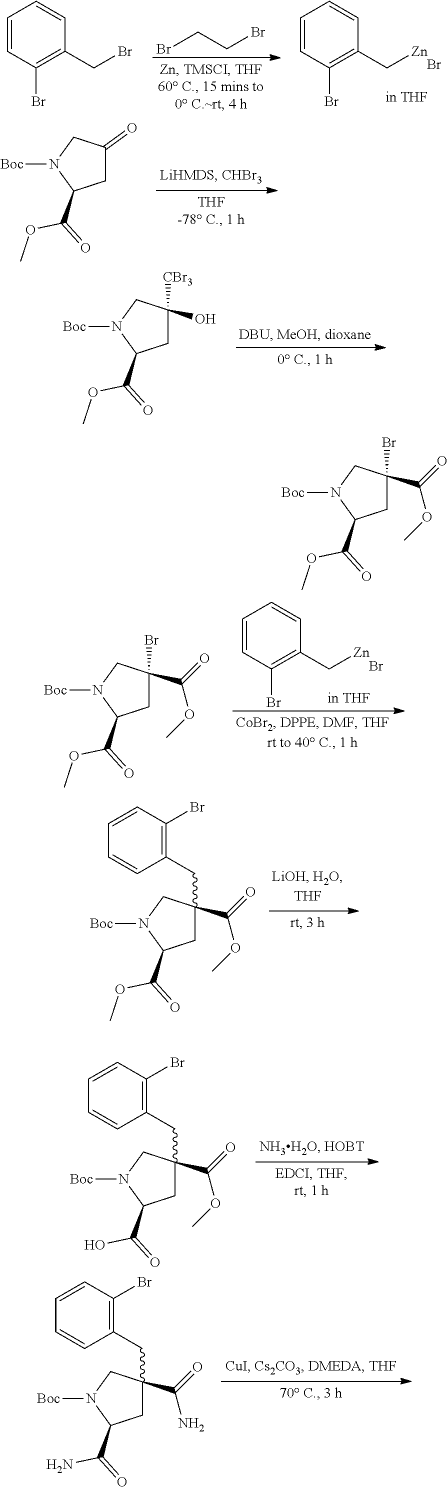

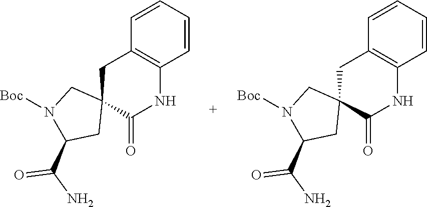 [SMILES: CC(C)(C)OC(=O)N1C[C@@]2(Cc3ccccc3NC2=O)C[C@H]1C(N)=O ; CC(C)(C)OC(=O)N1C[C@]2(Cc3ccccc3NC2=O)C[C@H]1C(N)=O]

To a stirred suspension of zinc dust (523 mg, 8.00 mmol) in THF (8 mL) was added 1,2-dibromoethane (37.6 mg, 0.20 mmol) dropwise at rt. The mixture was placed in an oil bath, heated to reflux and stirred for 10 min. Chlorotrimethylsilane (8.69 mg, 0.08 mmol) was added dropwise. The mixture was stirred at 60° C. for 15 mins. 1-bromo-2-(bromomethyl)benzene (1.00 g, 4.00 mmol) in THF (2.0 mL) was added slowly at 0° C. The mixture was stirred at rt for 4 h (monitored by TLC). The stirring was discontinued, and the unreacted zinc was allowed to settle. The mixture was cooled to rt to afford a solution of (2-bromobenzyl)zinc(II) bromide (0.4 mmol/mL in THF).

To a solution of 1-(t-butyl) 2-methyl (S)-4-oxopyrrolidine-1,2-dicarboxylate (1.00 g, 4.11 mmol) and tribromomethane (2.08 g, 8.22 mmol) in THF (10 mL) was added dropwise lithium bis(trimethylsilyl)amide (8.2 mL, 8.22 mmol, 1 M in THF) at −78° C. under nitrogen. The mixture was stirred for 1 h at −78° C. and the reaction was quenched with NH₄Cl (sat., aq., 100 mL). The mixture was extracted with EA (3×150 mL). The organic layers were combined, washed with brine (2×80 mL) and dried over anhydrous sodium sulfate. The solids were removed by filtration and the filtrate was concentrated under reduced pressure. The crude product was chromatographed on a silica gel column with EA:PE (35-45%) to provide 1-t-butyl 2-methyl (2S,4S)-4-hydroxy-4-(tribromomethyl)pyrrolidine-1,2-dicarboxylate (1.14 g, crude) as a light yellow solid. ¹H NMR (400 MHz, DMSO-d₆) δ 6.77-6.83 (m, 1H), 4.54-4.70 (m, 1H), 3.78-3.95 (m, 1H), 3.61-3.74 (m, 4H), 2.89-3.15 (m, 1H), 2.20-2.33 (m, 1H), 1.29-1.47 (m, 9H). LC-MS (ESI, m/z): 394 [M−100+H]⁺.

To a mixture of 1-(t-butyl) 2-methyl (2S,4S)-4-hydroxy-4-(tribromomethyl)pyrrolidine-1,2-dicarboxylate (500 mg, 1.01 mmol) and CH₃OH (113 mg, 3.55 mmol) in dioxane (5 mL) was added 1,8-diazabicyclo[5.4.0]undec-7-ene (339 mg, 2.23 mmol) at 0° C. The mixture was stirred for 1 h at 0° C. and the reaction was quenched with saturated ammonium chloride aqueous solution (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure. The crude product was chromatographed on a silica gel column with EA:PE (1:4) to provide 1-(t-butyl) 2,4-dimethyl (2S,4R)-4-bromopyrrolidine-1,2,4-tricarboxylate (170 mg, >90% pure, ~45% yield) as a light yellow oil. ¹H NMR (400 MHz, DMSO-d₆) δ 4.38-4.68 (m, 1H), 3.98-4.30 (m, 1H), 3.78-3.92 (m, 1H), 3.63-3.77 (m, 6H), 3.04-3.16 (m, 0.4H), 2.56-2.83 (m, 1.6H), 1.35-1.43 (m, 9H). LC-MS (ESI, m/z): 266 [M-Boc+H]⁺.

A solution of CoBr₂ (180 mg, 0.819 mmol) and 1,2-bis(diphenylphosphino)ethane (653 mg, 1.64 mmol) in DMF (30 mL) and THF (30 mL) was stirred for 15 mins at rt. 1-(t-butyl) 2,4-dimethyl (2S,4R)-4-bromopyrrolidine-1,2,4-tricarboxylate (3.00 g, 8.19 mmol) and the solution of (2-bromobenzyl)zinc(II) bromide (60.0 mL, 32.8 mmol) were added. The mixture was stirred for 1 h at 40° C. and the reaction was quenched with ammonium chloride solution (50 mL). The mixture was extracted with EA (3×200 mL). The organic layers were combined and dried over magnesium sulfate anhydrous. The solids were removed by filtration and the filtrate was concentrated under reduced pressure. The crude product was purified by C18 column with CH₃CN/Water (0.05% TFA), (68%). The fraction was concentrated under reduced pressure to provide 1-(t-butyl) 2,4-dimethyl (S)-4-(2-bromobenzyl)pyrrolidine-1,2,4-tricarboxylate (1.32 g, 35%) as a yellow oil. ¹H NMR (400 MHz, DMSO-d₆) δ 7.52-7.90 (m, 1H), 7.25-7.50 (m, 1H), 7.05-7.23 (m, 2H), 4.20-4.35 (m, 1H), 3.85-4.15 (m, 1H), 3.70-3.80 (m, 1H), 3.10-3.65 (m, 6H), 2.65-3.20 (m, 2H), 1.85-2.45 (m, 2H), 1.20-1.45 (m, 9H). LC-MS (ESI, m/z): 456 [M+H]⁺.

To a stirred mixture of 1-(t-butyl) 2,4-dimethyl (S)-4-(2-bromobenzyl)pyrrolidine-1,2,4-tricarboxylate (1.00 g, 1.97 mmol) in THF (10 mL) and water (10 mL) was added LiOH (262 mg, 9.86 mmol) at rt. The mixture was stirred for 3 h and acidified to pH=3 with HCl (2M). The mixture was extracted with EA (3×150 mL). The organic layers were combined and dried over anhydrous sodium sulfate. The organic layers was concentrated under reduced pressure to afford (S)-4-(2-bromobenzyl)-1-(t-butoxycarbonyl)pyrrolidine-2,4-dicarboxylic acid (1.00 g, crude) as a yellow oil. LC-MS (ESI, m/z): 428 [M+H]⁺.

To a mixture of (S)-4-(2-bromobenzyl)-1-(t-butoxycarbonyl)pyrrolidine-2,4-dicarboxylic acid (900 mg, 2.10 mmol) in THF (9 mL) were added 1-hydroxybenzotriazole (1.71 g, 12.6 mmol) and 1-(3-dimethylaminopropyl)-3-ethylcarbodiimide HCl (2.00 g, 10.5 mmol). The mixture was stirred for 30 mins at 0° C. and then ammonia (15 mL) was added at 0° C. The mixture was stirred for 2 h at rt and the reaction was quenched with water (30 mL). The mixture was extracted with EA (3×150 mL). The organic layers were combined, washed with brine (2×30 mL) and dried over magnesium sulfate anhydrous. The solids were removed by filtration and the filtrate was concentrated under reduced pressure to afford t-butyl (S)-4-(2-bromobenzyl)-2,4-dicarbamoylpyrrolidine-1-carboxylate (870 mg, crude) as a yellow solid. LC-MS (ESI, m/z): 426 [M+H]⁺.

To a mixture of t-butyl (S)-4-(2-bromobenzyl)-2,4-dicarbamoylpyrrolidine-1-carboxylate (770 mg, 1.81 mmol) in THF (8 mL) were added cuprous iodide (206 mg, 1.08 mmol), cesium carbonate (1.18 g, 3.61 mmol) and dimethylethylenediamine (303 mg, 3.43 mmol). The mixture was stirred for 3 h at 70° C. under nitrogen and the reaction was quenched with water (25 mL). The mixture was extracted with EA (3×150 mL). The organic layers were combined, washed with brine (2×30 mL) and dried over magnesium sulfate anhydrous. The solids were removed by filtration and the filtrate was concentrated under reduced pressure. The crude product was chromatographed on a silica gel column with CH₃OH:DCM (7:93) to provide t-butyl (3S,5S)-5-carbamoyl-2′-oxo-1′,4′-dihydro-2′H-spiro[pyrrolidine-3,3′-quinoline]-1-carboxylate (260 mg, 41%, isomer ratio: 1:2.3) as a yellow solid. The two isomers were separated by prep-HPLC (Column: XSelect CSH Prep C18 OBD, 30×150 mm, m; Mobile Phase A: Water (0.1% FA), Mobile Phase B: CH₃CN; Flow rate: 60 mL/min; Gradient: 26 to 65% B; Wave Length: 220 nm) to provide t-butyl (3R,5S)-5-carbamoyl-2′-oxo-1′,4′-dihydro-2′H-spiro[pyrrolidine-3,3′-quinoline]-1-carboxylate (48.0 mg) and t-butyl (3S,5S)-5-carbamoyl-2′-oxo-1′,4′-dihydro-2′H-spiro[pyrrolidine-3,3′-quinoline]-1-carboxylate (116.0 mg) as white solids.

### t-butyl (2R,5′S)-5′-carbamoyl-3-oxo-4,5-dihydro-3H-spiro[benzo[f][1,4]oxazepine-2,3′-pyrrolidine]-1′-carboxylate

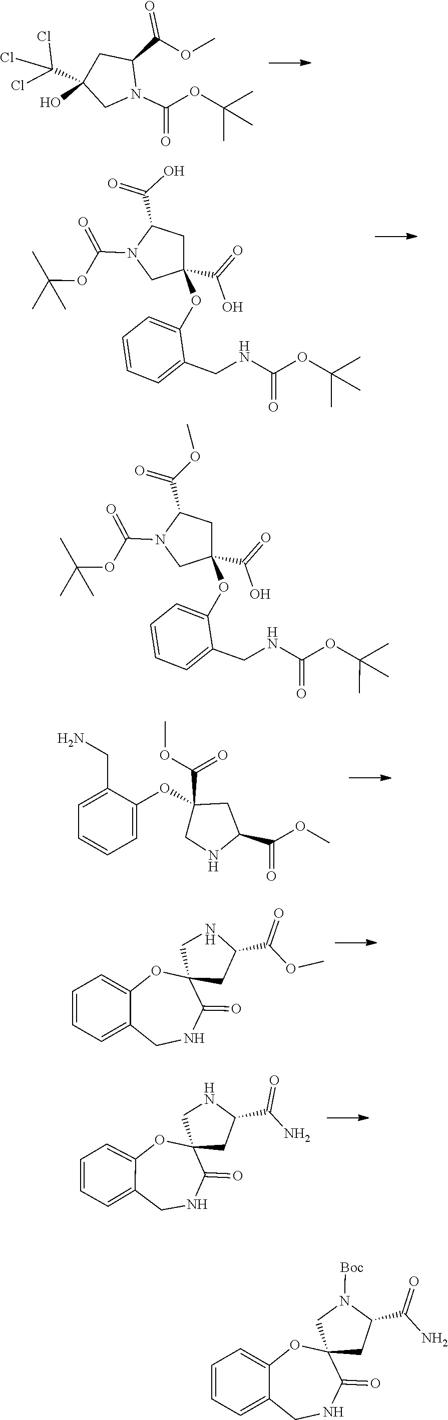

To a mixture of 1-(t-butyl) 2-methyl (2S,4S)-4-hydroxy-4-(trichloromethyl)pyrrolidine-1,2-dicarboxylate (2.00 g, 5.51 mmol) and t-butyl (2-hydroxybenzyl)carbamate (1.85 g, 8.27 mmol) in acetone (50 mL) was added NaOH (1.10 g, 27.6 mmol) at 0° C. The mixture was stirred overnight at rt and the reaction was quenched with water (100 mL). The mixture was adjusted to pH=5 with HCl (1 M) and extracted with EA (3×100 mL). The organic layers were combined, washed with brine (2×100 mL) and dried over anhydrous sodium sulfate. The solvents were removed by filtration and the solvate was concentrated under reduced pressure to afford (2S,4R)-1-(t-butoxycarbonyl)-4-(2-(((t-butoxycarbonyl)amino)methyl)phenoxy)pyrrolidine-2,4-dicarboxylic acid (2.66 g, crude) as a brown oil. LC-MS (ESI, m/z): 503 [M+Na]⁺.

To a mixture of (2S,4R)-1-(t-butoxycarbonyl)-4-(2-(((t-butoxycarbonyl)amino)methyl)phenoxy)pyrrolidine-2,4-dicarboxylic acid (2.66 g, 5.54 mmol) and potassium carbonate (3.08 g, 22.1 mmol) in DMF (30 mL) was added iodomethane (1.57 g, 11.1 mmol) at 0° C. The mixture was stirred for 1 h at rt and the reaction was quenched with water (80 mL). The mixture was extracted with EA (3×80 mL). The organic layers were combined, washed with brine (2×80 mL) and dried over anhydrous sodium sulfate. The solids were removed by filtration and the filtrate was concentrated under reduced pressure. The crude product was purified by C18 column with CH₃CN:Water (0.05% TFA). The fraction was concentrated under reduced pressure to provide 1-(t-butyl) 2,4-dimethyl (2S,4R)-4-(2-(((t-butoxycarbonyl)amino)methyl)phenoxy)pyrrolidine-1,2,4-tricarboxylate (1.00 g, 33%). ¹H NMR (400 MHz, DMSO-d₆) δ 7.10-7.28 (m, 3H), 6.96-7.06 (m, 1H), 6.50-6.58 (m, 1H), 4.34-4.46 (m, 1H), 4.05-4.16 (m, 2H), 3.95-4.01 (m, 1H), 3.78-3.88 (m, 1H), 3.61-3.77 (m, 6H), 2.78-2.89 (m, 1H), 2.51-2.56 (m, 1H), 1.26-1.45 (m, 18H). LC-MS (ESI, m/z): 531 [M+Na]⁺.

To a solution of 1-(t-butyl) 2,4-dimethyl (2S,4R)-4-(2-(((t-butoxycarbonyl)amino)methyl)phenoxy)pyrrolidine-1,2,4-tricarboxylate (500 mg, 0.983 mmol) in DCM (10 mL) was added TFA (3 mL). The mixture was stirred for 1 h at rt and concentrated under reduced pressure to afford dimethyl (2S,4R)-4-(2-(aminomethyl)phenoxy)pyrrolidine-2,4-dicarboxylate (303 mg, crude). LC-MS (ESI, m/z): 309 [M+H]⁺.

To a mixture of dimethyl (2S,4R)-4-(2-(aminomethyl)phenoxy)pyrrolidine-2,4-dicarboxylate (303 mg, 0.983 mmol) in MeOH (5 mL) was added N-ethyl-N-isopropylpropan-2-amine (381 mg, 2.95 mmol). The mixture was stirred for 2 h at rt and concentrated under reduced pressure to afford methyl (2R,5′S)-3-oxo-4,5-dihydro-3H-spiro[benzo[f][1,4]oxazepine-2,3′-pyrrolidine]-5′-carboxylate (272 mg, crude). LC-MS (ESI, m/z): 277 [M+H]⁺.

A mixture of methyl (2R,5′S)-3-oxo-4,5-dihydro-3H-spiro[benzo[f][1,4]oxazepine-2,3′-pyrrolidine]-5′-carboxylate (272 mg, 0.984 mmol) in ammonia (5 mL, 7 M in MeOH) was stirred overnight at 50° C. in a sealed vial. The mixture was concentrated under reduced pressure to afford (2R,5′S)-3-oxo-4,5-dihydro-3H-spiro[benzo[f][1,4]oxazepine-2,3′-pyrrolidine]-5′-carboxamide (257 mg, crude). LC-MS (ESI, m/z): 262 [M+H]⁺.

To a mixture of (2R,5′S)-3-oxo-4,5-dihydro-3H-spiro[benzo[f][1,4]oxazepine-2,3′-pyrrolidine]-5′-carboxamide (257 mg, 0.984 mmol) and di-t-butyl dicarbonate (258 mg, 1.18 mmol) in DCM (5 mL) was added NEt₃ (199 mg, 1.97 mmol). The mixture was stirred for 2 h at rt and concentrated under reduced pressure. The crude product was chromatographed on a silica gel column with MeOH:DCM (6:94) to provide t-butyl (2R,5′S)-5′-carbamoyl-3-oxo-4,5-dihydro-3H-spiro[benzo[f][1,4]oxazepine-2,3′-pyrrolidine]-1′-carboxylate (105 mg, 28%) as an off-white solid. ¹H NMR (400 MHz, DMSO-d₆) δ 8.48-8.56 (m, 1H), 7.48-7.57 (m, 1H), 7.33-7.41 (m, 2H), 7.14-7.23 (m, 1H), 6.99-7.09 (m, 1H), 6.90-6.97 (m, 1H), 4.11-4.38 (m, 3H), 3.74-3.87 (m, 1H), 3.54-3.62 (m, 1H), 2.51-2.57 (m, 1H), 2.29-2.40 (m, 1H), 1.41 (s, 9H). LC-MS (ESI, m/z): 362 [M+H]⁺.

### tert-butyl (3S,5R)-3-carbamoyl-6-oxo-8-phenyl-2,7-diazaspiro[4.4]non-8-ene-2-carboxylate

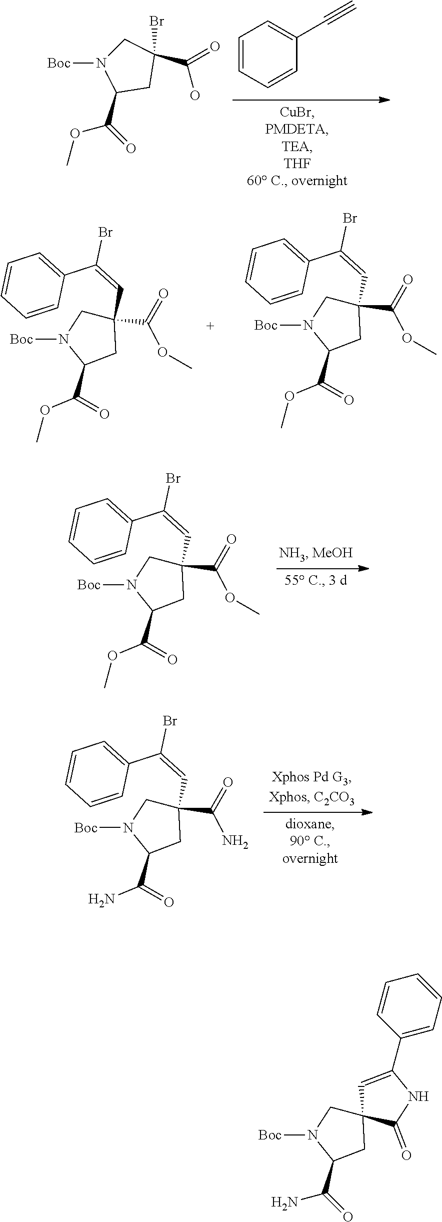

To a mixture of 1-(t-butyl) 2,4-dimethyl (2S,4R)-4-bromopyrrolidine-1,2,4-tricarboxylate (3.00 g, 8.19 mmol) in THF (60 mL) were added ethynylbenzene (2.51 g, 24.6 mmol), copper(I) bromide (118 mg, 0.819 mmol), pentamethyldiethylenetriamine (142 mg, 0.819 mmol) and NEt₃ (1.25 g, 12.3 mmol). The mixture was stirred overnight at 60° C. under nitrogen and the reaction was quenched with water (100 mL). The mixture was extracted with EA (3×100 mL). The organic layers were combined, washed with brine (2×100 mL) and dried over anhydrous sodium sulfate. The solids were removed by filtration and the filtrate was concentrated under reduced pressure. The crude product was chromatographed on a silica gel column with EA:PE (1:3) to provide Isomer 1: 1-(t-butyl) 2,4-dimethyl (2S,4S)-4-((E)-2-bromo-2-phenylvinyl)pyrrolidine-1,2,4-tricarboxylate (740 mg, 13%) as a light yellow oil. ¹H NMR (400 MHz, DMSO-d₆) δ 7.38-7.42 (m, 3H), 7.21-7.28 (m, 2H), 6.40-6.53 (m, 1H), 4.17-4.33 (m, 1H), 3.97-4.11 (m, 1H), 3.77-3.93 (m, 1H), 3.30-3.46 (m, 6H), 2.56-2.67 (m, 1H), 2.22-2.45 (m, 1H), 1.27-1.33 (m, 9H). LC-MS (ESI, m/z): 368 [M−100+H]⁺; and Isomer 2: 1-(t-butyl) 2,4-dimethyl (2S,4R)-4-((E)-2-bromo-2-phenylvinyl)pyrrolidine-1,2,4-tricarboxylate (1.00 g, 20%) as a light yellow oil. ¹H NMR (400 MHz, DMSO-d₆) δ 7.39-7.43 (m, 3H), 7.22-7.27 (m, 2H), 6.39-6.52 (m, 1H), 4.54-4.76 (m, 1H), 4.20-4.27 (m, 1H), 3.79-3.89 (m, 1H), 3.30-3.41 (m, 6H), 2.57-2.66 (m, 1H), 2.27-2.38 (m, 1H), 1.28-1.35 (m, 9H). LC-MS (ESI, m/z): 368 [M−100+H]⁺.

A mixture of 1-(t-butyl) 2,4-dimethyl (2S,4R)-4-((E)-2-bromo-2-phenylvinyl)pyrrolidine-1,2,4-tricarboxylate (1.00 g, 2.14 mmol) in ammonia (60 mL, 7 M in MeOH) was stirred for 3 days at 55° C. The mixture was concentrated under reduced pressure. The crude product was chromatographed on a silica gel column with MeOH:DCM (1:24) to provide t-butyl (2S,4R)-4-((E)-2-bromo-2-phenylvinyl)-2,4-dicarbamoylpyrrolidine-1-carboxylate (500 mg, 41%) as a light yellow solid. ¹H NMR (400 MHz, DMSO-d₆) δ 7.34-7.67 (m, 4H), 7.12-7.33 (m, 4H), 6.84-7.04 (m, 1H), 6.32-6.47 (m, 1H), 3.87-3.99 (m, 1H), 3.40-3.65 (m, 1H), 3.13-3.29 (m, 1H), 2.04-2.24 (m, 1H), 1.89-2.03 (m, 1H), 1.28-1.44 (m, 9H). LC-MS (ESI, m/z): 438 [M+H]⁺.

To a mixture of t-butyl (2S,4R)-4-((E)-2-bromo-2-phenylvinyl)-2,4-dicarbamoylpyrrolidine-1-carboxylate (500 mg, 1.14 mmol), 2-(dicyclohexylphosphino)-2′,4′,6′-triisopropylbiphenyl (164 mg, 0.343 mmol) and methanesulfonato(2-dicyclohexylphosphino-2′,4′,6′-tri-1-propyl-1,1′-biphenyl)(2′-amino-1,1′-biphenyl-2-yl)palladium(II) (386 mg, 0.456 mmol) in dioxane (15 mL) was added cesium carbonate (1.49 g, 4.56 mmol). The mixture was stirred overnight at 90° C. under nitrogen and the reaction was quenched with water (30 mL). The mixture was extracted with EA (3×30 mL). The organic layers were combined, washed with brine (2×30 mL) and dried over anhydrous sodium sulfate. The solids were removed by filtration and the filtrate was concentrated under reduced pressure. The crude product was purified by TLC (Mobile phase: MeOH:DCM=1:10; Rf=0.4; detection: UV) to provide t-butyl (3S,5R)-3-carbamoyl-6-oxo-8-phenyl-2,7-diazaspiro[4.4]non-8-ene-2-carboxylate (120 mg, 23%) as a light yellow solid. ¹H NMR (400 MHz, DMSO-d₆) δ 7.61-7.70 (m, 1H), 7.37-7.54 (m, 4H), 7.12-7.29 (m, 1H), 6.85-7.09 (m, 2H), 6.36-6.71 (m, 1H), 4.18-4.35 (m, 1H), 3.84-3.95 (m, 1H), 3.48-3.61 (m, 1H), 2.18-2.31 (m, 1H), 1.88-2.07 (m, 1H), 1.15-1.48 (m, 9H). LC-MS (ESI, m/z): 358 [M+H]⁺.

### (3S,5R)-6-oxo-8-(pyridin-3-yl)-2,7-diazaspiro[4.4]non-8-ene-3-carboxamide

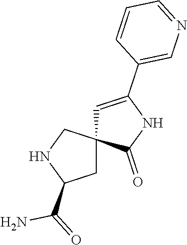 [SMILES: NC(=O)[C@@H]1C[C@@]2(C=C(c3cccnc3)NC2=O)CN1]

To a mixture of 1-(t-butyl) 2,4-dimethyl (2S,4R)-4-bromopyrrolidine-1,2,4-tricarboxylate (3.00 g, 8.22 mmol) in THF (60 ml) were added 3-ethynylpyridine (2.53 g, 24.5 mmol), copper(I) bromide (117 mg, 0.822 mmol), (2-[[2-(dimethylamino)ethyl](methyl)amino]ethyl)dimethylamine (142 mg, 0.822 mmol) and trimethylamine (1.24 g, 12.2 mmol). The mixture was stirred overnight at 60° C. under nitrogen and the reaction was quenched with water (100 mL). The mixture was extracted with EtOAc (3×100 mL). The organic layers were combined, washed with brine (2×100 mL), dried over anhydrous sodium sulfate, filtered and concentrated under reduced pressure to remove the solvent. The residue was chromatographed on a silica gel column with EtOAc:PE (3:10) to afford the crude product (1.1 g) and then purified by TLC (Mobile phase: EtOAc:PE=1:3; Rf1=0.4, Rf2=0.3; detection: UV). Purification resulted in isomer 1: 1-(t-butyl) 2,4-dimethyl (2S,4S)-4-((E)-2-bromo-2-(pyridin-3-yl)vinyl)pyrrolidine-1,2,4-tricarboxylate (180 mg, 4%) as a yellow oil. ¹H NMR (400 MHz, DMSO-d₆) δ 8.54-8.65 (m, 1H), 8.38-8.49 (m, 1H), 7.65-7.77 (m, 1H), 7.42-7.55 (m, 1H), 6.58-6.72 (m, 1H), 3.55-3.75 (m, 6H), 3.35-3.43 (m, 3H), 2.60-2.70 (m, 1H), 2.01-2.11 (m, 1H), 1.25-1.35 (m, 9H). LC-MS (ESI, m/z): 469 [M+H]⁺, and isomer 2: 1-(t-butyl) 2,4-dimethyl (2S,4R)-4-((E)-2-bromo-2-(pyridin-3-yl)vinyl)pyrrolidine-1,2,4-tricarboxylate (630 mg, 15%) as a yellow oil. ¹H NMR (400 MHz, DMSO-d₆) δ 8.54-8.60 (m, 1H), 8.39-8.47 (m, 1H), 7.68-7.74 (m, 1H), 7.43-7.52 (m, 1H), 6.54-6.64 (m, 1H), 4.21-4.30 (m, 1H), 3.54-3.74 (m, 6H), 3.35-3.44 (m, 2H), 2.28-2.47 (m, 2H), 1.28-1.38 (m, 9H). LC-MS (ESI, m/z): 413 [M−56+H]⁺.

A mixture of 1-(t-butyl) 2,4-dimethyl (2S,4R)-4-((E)-2-bromo-2-(pyridin-3-yl)vinyl)pyrrolidine-1,2,4-tricarboxylate (630 mg, 1.35 mmol) in ammonia (20 mL, 7 M in MeOH) was stirred overnight at 60° C. The mixture was concentrated under reduced pressure to afford t-butyl (2S,4R)-4-((E)-2-bromo-2-(pyridin-3-yl)vinyl)-2,4-dicarbamoylpyrrolidine-1-carboxylate (590 mg, crude) as a yellow solid. LC-MS (ESI, m/z): 439 [M+H]⁺.

To a mixture of t-butyl (2S,4R)-4-((E)-2-bromo-2-(pyridin-3-yl)vinyl)-2,4-dicarbamoylpyrrolidine-1-carboxylate (590 mg, 1.35 mmol), dicyclohexyl(3-isopropoxy-2′,4′,6′-triisopropyl-[1,1′-biphenyl]-2-yl)phosphane (144 mg, 0.269 mmol) and methanesulfonato{Dicyclohexyl[3-(1-methylethoxy)-2′,4′,6′-tris(1-methylethyl)-1,1′-biphenyl-2-yl]phosphine}(2′-methylamino-1,1′-biphenyl-2-yl)palladium(II) (185 mg, 0.202 mmol) in dioxane (10 mL) was added cesium carbonate (878 mg, 2.69 mmol). The mixture was stirred overnight at 80° C. under nitrogen and the reaction was quenched with water (30 mL). The mixture was extracted with EtOAc (3×30 mL). The organic layers were combined, washed with brine (2×30 mL), dried over anhydrous sodium sulfate, filtered and concentrated under reduced pressure to afford the crude product. The crude product was chromatographed on a silica gel column with MeOH:DCM (1:12) to provide t-butyl (3S,5R)-3-carbamoyl-6-oxo-8-(pyridin-3-yl)-2,7-diazaspiro[4.4]non-8-ene-2-carboxylate (170 mg, 35%) as a yellow solid. ¹H NMR (400 MHz, DMSO-d₆) δ 8.53-8.67 (m, 2H), 7.82-7.90 (m, 1H), 7.63-7.74 (m, 1H), 7.42-7.57 (m, 3H), 6.97-7.12 (m, 1H), 4.23-4.36 (m, 1H), 3.89-3.98 (m, 1H), 3.52-3.62 (m, 1H), 2.54-2.60 (m, 1H), 2.24-2.34 (m, 1H), 1.34-1.42 (m, 9H). LC-MS (ESI, m/z): 359 [M+H]⁺.

A mixture of t-butyl (3S,5R)-3-carbamoyl-6-oxo-8-(pyridin-3-yl)-2,7-diazaspiro[4.4]non-8-ene-2-carboxylate (170 mg, 0.475 mmol) in HCl (3 mL, 4.0 M in 1,4-dioxane) was stirred for 1 h at rt and concentrated under reduced pressure to afford (3S,5R)-6-oxo-8-(pyridin-3-yl)-2,7-diazaspiro[4.4]non-8-ene-3-carboxamide (122 mg, crude) as a yellow solid. LC-MS (ESI, m/z): 259 [M+H]⁺.

### t-butyl (5′S)-5′-carbamoyl-2-oxohexahydro-2H-spiro[cyclopenta[b]pyrrole-3,3′-pyrrolidine]-1′-carboxylate

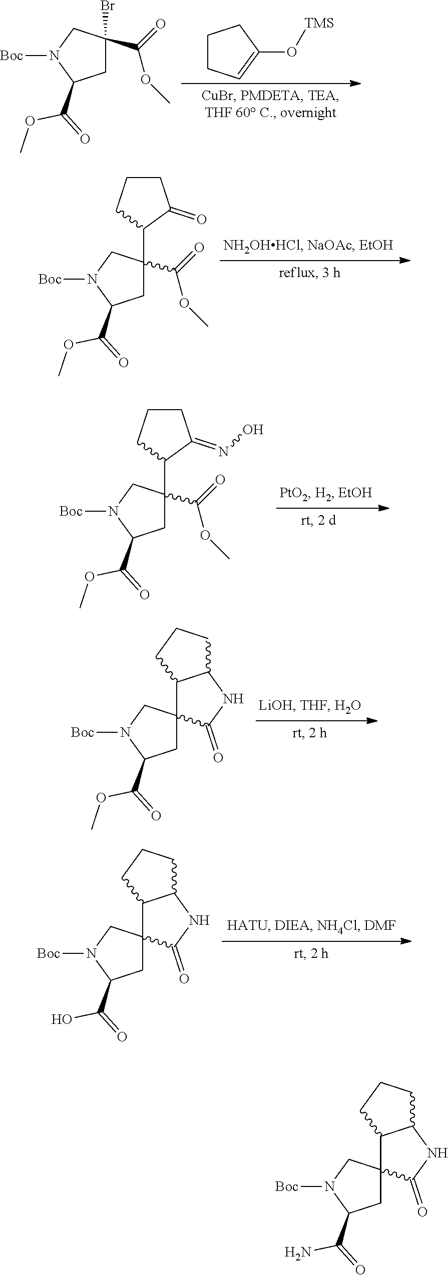

To a mixture of 1-(t-butyl) 2,4-dimethyl (2S,4R)-4-bromopyrrolidine-1,2,4-tricarboxylate (3.00 g, 8.19 mmol) in THF (45 mL) WERE added (cyclopent-1-en-1-yloxy)trimethylsilane (3.84 g, 24.5 mmol), N¹-(2-(dimethylamino)ethyl)-N¹,N²,N²-trimethylethane-1,2-diamine (141 mg, 0.819 mmol), NEt₃ (1.24 g, 12.2 mmol) and cuprous bromide (117 mg, 0.819 mmol). The mixture was stirred overnight at 60° C. under nitrogen and the reaction was quenched with water (100 mL). The mixture was extracted with EA (3×100 mL). The organic layers were combined, washed with brine (1×100 mL) and dried over anhydrous sodium sulfate. The solids were removed by filtration and the filtrate was concentrated under reduced pressure to remove the solvent. The residue was chromatographed on a silica gel column with EA:PE (35:65) to afford the crude product 1-(t-butyl) 2,4-dimethyl (2S)-4-(2-oxocyclopentyl)pyrrolidine-1,2,4-tricarboxylate (1.1 g, crude) as a light yellow oil. LC-MS (ESI, m/z): 392 [M+Na]⁺.

To a mixture of 1-(t-butyl) 2,4-dimethyl (2S)-4-(2-oxocyclopentyl)pyrrolidine-1,2,4-tricarboxylate (1.10 g, 2.97 mmol) in EtOH (30 mL) were added hydroxylamine hydrochloride (1.03 g, 14.8 mmol) and sodium acetate (1.22 g, 14.8 mmol). The mixture was reflux for 3 h and the reaction was quenched with water (60 mL). The mixture was extracted with EA (3×50 mL). The organic layers were combined, washed with brine (2×30 mL) and dried over anhydrous sodium sulfate. The solids were removed by filtration and the filtrate was concentrated under reduced pressure to afford 1-(t-butyl) 2,4-dimethyl (2S)-4-(-2-(hydroxyimino)cyclopentyl)pyrrolidine-1,2,4-tricarboxylate (800 mg, crude) as a yellow oil. LC-MS (ESI, m/z): 407 [M+Na]⁺.

To a mixture of 1-(t-butyl) 2,4-dimethyl (2S)-4-((E)-2-(hydroxyimino)cyclopentyl)pyrrolidine-1,2,4-tricarboxylate (800 mg, 2.08 mmol) in EtOH (20 mL) was added platinum dioxide (470 mg, 2.08 mmol). The mixture was stirred for 2 days at rt under hydrogen atmosphere. The mixture was filtered through a celite pad and the filtrate was concentrated under reduced pressure. The crude product was chromatographed on a silica gel column with EA:PE (85:15) to provide 1′-(t-butyl) 5′-methyl (5′S)-2-oxohexahydro-2H-spiro[cyclopenta[b]pyrrole-3,3′-pyrrolidine]-1′,5′-dicarboxylate (150 mg, 5% for 3 steps) as a yellow oil. ¹H NMR (400 MHz, DMSO-d₆) δ 7.86 (s, 1H), 4.15-4.26 (m, 1H), 3.91-4.01 (m, 1H), 3.62-3.72 (m, 3H), 3.45-3.54 (m, 1H), 3.33-3.40 (m, 1H), 2.25-2.48 (m, 2H), 1.42-1.82 (m, 7H), 1.29-1.38 (m, 9H). LC-MS (ESI, m/z): 339 [M+H]⁺.

To a mixture of 1′-(t-butyl) 5′-methyl (5′S)-2-oxohexahydro-2H-spiro[cyclopenta[b]pyrrole-3,3′-pyrrolidine]-1′,5′-dicarboxylate (150 mg, 0.443 mmol) in THF (2 mL) was added a solution of LiOH (31.0 mg, 1.32 mmol) in H₂O (2 mL). The mixture was stirred for 2 h at rt and the reaction was quenched with water (5 mL). The mixture was acidified to pH=3 with HCl (1 M). The mixture was extracted with EA (3×10 mL). The organic layers were combined and dried over anhydrous sodium sulfate. The solids were removed by filtration and the filtrate was concentrated under reduced pressure to afford (5′S)-1′-(t-butoxycarbonyl)-2-oxohexahydro-2H-spiro[cyclopenta[b]pyrrole-3,3′-pyrrolidine]-5′-carboxylic acid (80.0 mg, crude) as a light yellow solid. ¹H NMR (400 MHz, DMSO-d₆) δ 12.58 (br, 1H), 7.81-7.89 (m, 1H), 3.95-4.12 (m, 2H), 3.43-3.52 (m, 1H), 3.33-3.38 (m, 1H), 2.28-2.48 (m, 2H), 1.44-1.85 (m, 7H), 1.30-1.38 (m, 9H). LC-MS (ESI, m/z): 325 [M+H]⁺.

To a mixture of (5′S)-1′-(t-butoxycarbonyl)-2-oxohexahydro-2H-spiro[cyclopenta[b]pyrrole-3,3′-pyrrolidine]-5′-carboxylic acid (80.0 mg, 0.247 mmol), ammonium chloride (92.0 mg, 1.72 mmol) and HATU (112 mg, 0.296 mmol) in DMF (3 mL) was added DIPEA (191 mg, 1.48 mmol) at 0° C. The mixture was stirred for 2 h at rt. The mixture was purified by C18 column with CH₃CN/Water (0.05% TFA), and the fraction was concentrated under reduced pressure to provide t-butyl (5′S)-5′-carbamoyl-2-oxohexahydro-2H-spiro[cyclopenta[b]pyrrole-3,3′-pyrrolidine]-1′-carboxylate (50.0 mg, 56%) as a light yellow solid. ¹H NMR (400 MHz, DMSO-d₆) δ 7.82 (s, 1H), 7.34-7.43 (m, 1H), 6.91-6.99 (m, 1H), 3.96-4.05 (m, 2H), 3.58-3.67 (m, 1H), 3.12-3.20 (m, 1H), 2.30-2.44 (m, 2H), 1.50-1.72 (m, 7H), 1.31-1.38 (m, 9H). LC-MS (ESI, m/z): 324 [M+H]⁺.

### (3R,5S)-2′-oxo-1′,2′-dihydrospiro[pyrrolidine-3,3′-pyrrolo[2,3-b]pyridine]-5-carboxamide

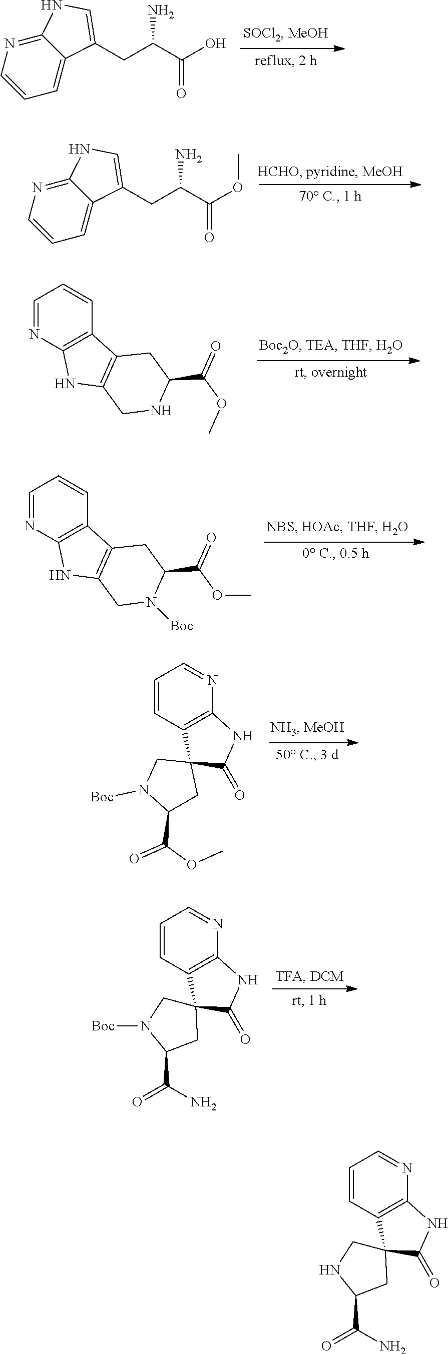

To a mixture of (S)-2-amino-3-(1H-pyrrolo[2,3-b]pyridin-3-yl)propanoic acid (2.00 g, 9.74 mmol) in MeOH (34 mL) was added thionyl chloride (3.48 g, 29.2 mmol) at 0° C. After stirring for 10 mins at rt, the mixture was stirred for 2 h at 70° C. The mixture was concentrated under reduced pressure to afford methyl (S)-2-amino-3-(1H-pyrrolo[2,3-b]pyridin-3-yl)propanoate (2.13 g, crude) as an off-white solid. LC-MS (ESI, m/z): 220 [M+H]⁺.

To a mixture of methyl (S)-2-amino-3-(1H-pyrrolo[2,3-b]pyridin-3-yl)propanoate (2.13 g, 9.71 mmol) in pyridine (24 mL)/MeOH (8 mL) was added formaldehyde (0.8 mL, 37% in H₂O). The mixture was stirred for 1 h at 70° C. and concentrated under reduced pressure to remove the solvent. Pyridine (30 mL) was added to the residue, heated to 70° C. to dissolve it, and then cooled in an ice bath to precipitate the solid. The solid was filtered and dried to obtain the methyl (S)-6,7,8,9-tetrahydro-5H-pyrrolo[2,3-b:5,4-c′]dipyridine-6-carboxylate (2.25 g, crude) as an off-white solid. LC-MS (ESI, m/z): 232 [M+H]⁺.

To a mixture of (S)-6,7,8,9-tetrahydro-5H-pyrrolo[2,3-b:5,4-c′]dipyridine-6-carboxylate (2.25 g, 9.73 mmol) in THF (20 mL)/H₂O (5 mL) was added triethylamine (2.95 g, 29.0 mmol) and di-t-butyl dicarbonate (5.31 g, 24.3 mmol) at 0° C. The mixture was stirred overnight at rt and the reaction was quenched with water (50 mL). The mixture was extracted with EtOAc (3×50 mL). The organic layers were combined, washed with brine (2×50 mL), dried over anhydrous sodium sulfate, filtered and concentrated under reduced pressure to afford the crude product. The crude product was chromatographed on a silica gel column with EtOAc:PE (51:49) to provide 7-(t-butyl) 6-methyl (S)-5,6,8,9-tetrahydro-7H-pyrrolo[2,3-b:5,4-c′]dipyridine-6,7-dicarboxylate (1.12 g, 33%) as a light yellow solid. ¹H NMR (400 MHz, DMSO-d₆) δ 11.27-11.54 (m, 1H), 8.07-8.22 (m, 1H), 7.79-7.96 (m, 1H), 6.94-7.09 (m, 1H), 5.11-5.34 (m, 1H), 4.69-4.81 (m, 1H), 4.27-4.50 (m, 1H), 3.51-3.63 (m, 3H), 3.24-3.31 (m, 1H), 2.94-3.09 (m, 1H), 1.32-1.68 (m, 9H). LC-MS (ESI, m/z): 332 [M+H]⁺.

To a mixture of 7-(t-butyl) 6-methyl (S)-5,6,8,9-tetrahydro-7H-pyrrolo[2,3-b:5,4-c′]dipyridine-6,7-dicarboxylate (1.12 g, 3.38 mmol) in THF (16 mL)/H₂O (2 mL) were added acetic acid (1.42 g, 23.7 mmol) and N-bromosuccinimide (601 mg, 3.38 mmol) at 0° C. The mixture was stirred for 0.5 h at 0° C. and the reaction was quenched with a solution of potassium carbonate (1.63 g, 11.8 mmol) in cold water (30 mL). The mixture was extracted with EtOAc (3×30 mL). The organic layers were combined, washed with saturated aqueous sodium bicarbonate solution (30 mL), brine (2×30 mL), dried over anhydrous sodium sulfate, filtered and concentrated under reduced pressure to afford the crude product. The crude product was chromatographed on a silica gel column with EtOAc:PE (44:56) to provide 1-(t-butyl) 5-methyl (3R,5S)-2′-oxo-1′,2′-dihydrospiro[pyrrolidine-3,3′-pyrrolo[2,3-b]pyridine]-1,5-dicarboxylate (1.05 g, 89%) as a light yellow solid. ¹H NMR (400 MHz, DMSO-d₆) δ 10.96-11.41 (m, 1H), 7.98-8.27 (m, 1H), 7.33-7.59 (m, 1H), 6.89-7.09 (m, 1H), 4.48-4.74 (m, 1H), 3.69-3.81 (m, 3H), 3.58-3.67 (m, 1H), 3.48-3.56 (m, 1H), 2.58-2.67 (m, 1H), 2.18-2.37 (m, 1H), 1.28-1.57 (m, 9H). LC-MS (ESI, m/z): 348 [M+H]⁺.

A mixture of 1-(t-butyl) 5-methyl (3R,5S)-2′-oxo-1′,2′-dihydrospiro[pyrrolidine-3,3′-pyrrolo[2,3-b]pyridine]-1,5-dicarboxylate (1.00 g, 2.87 mmol) in ammonia (30 mL, 7 M in MeOH) was stirred for 3 days at 50° C. The mixture was concentrated under reduced pressure to afford the crude product. The crude product was purified by prep-HPLC (Column: XSelect CSH Prep C18 OBD, 5 μm, 19×150 mm; Mobile Phase A: Water (10 mmol/L NH₄HCO₃), Mobile Phase B: ACN; Flow rate: 25 mL/min; Gradient: 25% to 70% B in 7 min; 254 nm; Rt: 5.30 min) to afford t-butyl (3R,5S)-5-carbamoyl-2′-oxo-1′,2′-dihydrospiro[pyrrolidine-3,3′-pyrrolo[2,3-b]pyridine]-1-carboxylate (290 mg, 28%) as an off-white solid. ¹H NMR (400 MHz, DMSO-d₆) δ 10.60 (br, 1H), 8.04-8.27 (m, 1H), 7.46-7.56 (m, 1H), 7.22-7.33 (m, 1H), 7.07-7.18 (m, 1H), 6.98-7.06 (m, 1H), 4.36-4.49 (m, 1H), 3.59-3.72 (m, 1H), 3.48-3.58 (m, 1H), 2.15-2.37 (m, 2H), 1.18-1.59 (m, 9H). LC-MS (ESI, m/z): 333 [M+H]⁺.

To a mixture of t-butyl (3R,5S)-5-carbamoyl-2′-oxo-1′,2′-dihydrospiro[pyrrolidine-3,3′-pyrrolo[2,3-b]pyridine]-1-carboxylate (150 mg, 0.452 mmol) in DCM (3 mL) was added TFA (1 mL). The mixture was stirred for 1 h at rt and concentrated under reduced pressure to afford (3R,5S)-2′-oxo-1′,2′-dihydrospiro[pyrrolidine-3,3′-pyrrolo[2,3-b]pyridine]-5-carboxamide (105 mg, crude) as a light yellow oil. LC-MS (ESI, m/z): 233 [M+H]⁺.

### (3R,5′S)-7-fluoro-2-oxospiro[indoline-3,3′-pyrrolidine]-5′-carboxamide

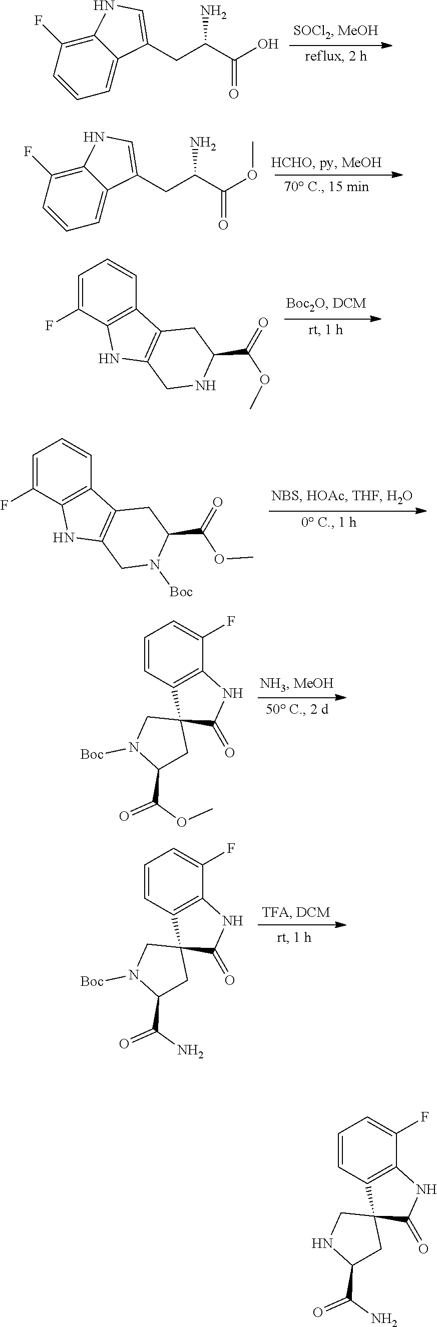

To a mixture of (S)-2-amino-3-(7-fluoro-1H-indol-3-yl)propanoic acid (1.60 g, 7.20 mmol) in MeOH (30 mL) was added thionyl chloride (2.57 g, 21.6 mmol) at 0° C. The mixture was reflux for 2 h and concentrated under reduced pressure to afford methyl (S)-2-amino-3-(7-fluoro-1H-indol-3-yl)propanoate (1.69 g, crude) as an off-white solid. LC-MS (ESI, m/z): 237 [M+H]⁺.

To a mixture of methyl (S)-2-amino-3-(7-fluoro-1H-indol-3-yl)propanoate (1.69 g, 7.15 mmol) in pyridine (19.2 mL) and MeOH (6.4 mL) was added formaldehyde (639 mg, 7.86 mmol, 37% in H₂O). The mixture was stirred for 15 min at 70° C. and the reaction was quenched with water (50 mL). The mixture was extracted with EtOAc (3×50 mL). The organic layers were combined, washed with brine (2×50 mL), dried over anhydrous sodium sulfate, filtered and concentrated under reduced pressure to afford methyl (S)-8-fluoro-2,3,4,9-tetrahydro-1H-pyrido[3,4-b]indole-3-carboxylate (1.77 g, crude) as a yellow solid. LC-MS (ESI, m/z): 249 [M+H]⁺.

To a mixture methyl (S)-8-fluoro-2,3,4,9-tetrahydro-1H-pyrido[3,4-b]indole-3-carboxylate (1.77 g, 7.15 mmol) in DCM (25 mL) was added di-t-butyl dicarbonate (3.90 g, 17.9 mmol). The mixture was stirred for 1 h at rt and the reaction was quenched with water (50 mL). The mixture was extracted with DCM (3×50 mL). The organic layers were combined, washed with brine (2×50 mL), dried over anhydrous sodium sulfate, filtered and concentrated under reduced pressure to afford the crude product. The crude product was chromatographed on a silica gel column with EtOAc:PE (3:7) to provide 2-(t-butyl) 3-methyl (S)-8-fluoro-1,3,4,9-tetrahydro-2H-pyrido[3,4-b]indole-2,3-dicarboxylate (1.75 g, 70% yield for 3 steps) as a yellow solid. ¹H NMR (400 MHz, DMSO-d₆) δ 11.24-11.48 (m, 1H), 7.20-7.29 (m, 1H), 6.82-7.06 (m, 2H), 5.17-5.29 (m, 1H), 4.67-4.83 (m, 1H), 4.27-4.52 (m, 1H), 3.50-3.64 (m, 3H), 3.24-3.31 (m, 1H), 2.92-3.08 (m, 1H), 1.38-1.52 (m, 9H). LC-MS (ESI, m/z): 349 [M+H]⁺.

To a mixture of 2-(t-butyl) 3-methyl (S)-8-fluoro-1,3,4,9-tetrahydro-2H-pyrido[3,4-b]indole-2,3-dicarboxylate (1.70 g, 4.88 mmol) in H₂O (3.5 mL)/THF (28 mL) were added acetic acid (2.05 g, 34.2 mmol) and N-Bromosuccinimide (870 mg, 4.88 mmol) at 0° C. The mixture was stirred for 1 h at 0° C. and the reaction was quenched with a solution of potassium carbonate (2.36 g, 17.1 mmol) in cold water (50 mL). The mixture was extracted with EtOAc (3×50 mL). The organic layers were combined, washed with saturated aqueous sodium bicarbonate solution (50 mL), brine (2×50 mL), dried over anhydrous sodium sulfate, filtered and concentrated under reduced pressure to afford the crude product. The crude product was chromatographed on a silica gel column with EtOAc:PE (1:3) to provide 1′-(t-butyl) 5′-methyl (3R,5′S)-7-fluoro-2-oxospiro[indoline-3,3′-pyrrolidine]-1′,5′-dicarboxylate (1.33 g, 74%) as a yellow solid. ¹H NMR (400 MHz, DMSO-d₆) δ 11.00-11.20 (m, 1H), 7.13-7.26 (m, 1H), 6.99-7.11 (m, 1H), 6.89-6.98 (m, 1H), 4.57-4.69 (m, 1H), 3.69-3.79 (m, 3H), 3.57-3.67 (m, 1H), 3.49-3.56 (m, 1H), 2.36-2.49 (m, 1H), 2.17-2.33 (m, 1H), 1.31-1.44 (m, 9H). LC-MS (ESI, m/z): 365 [M+H]⁺.

A mixture of 1′-(t-butyl) 5′-methyl (3R,5′S)-7-fluoro-2-oxospiro[indoline-3,3′-pyrrolidine]-1′,5′-dicarboxylate (1.33 g, 3.65 mmol) in ammonia (50 mL, 7 M in MeOH) was stirred for 2 days at 50° C. The mixture was concentrated under reduced pressure to afford the crude product. The crude product was purified by C18 column with CH₃CN/Water (0.05% TFA). The fraction was concentrated under reduced pressure to provide t-butyl (3R,5′S)-5′-carbamoyl-7-fluoro-2-oxospiro[indoline-3,3′-pyrrolidine]-1′-carboxylate (1.00 g, 78%) as a light yellow solid. ¹H NMR (400 MHz, DMSO-d₆) δ 11.16 (s, 1H), 7.49-7.57 (m, 1H), 7.07-7.29 (m, 2H), 7.00-7.06 (m, 1H), 6.73-6.84 (m, 1H), 4.37-4.49 (m, 1H), 3.62-3.69 (m, 1H), 3.49-3.60 (m, 1H), 2.18-2.34 (m, 2H), 1.36-1.47 (m, 9H). LC-MS (ESI, m/z): 350 [M+H]⁺.

To a mixture of t-butyl (3R,5′S)-5′-carbamoyl-7-fluoro-2-oxospiro[indoline-3,3′-pyrrolidine]-1′-carboxylate (100 mg, 0.286 mmol) in DCM (3 mL) was added TFA (1 mL). The mixture was stirred for 1 h at rt and concentrated under reduced pressure to afford (3R,5′S)-7-fluoro-2-oxospiro[indoline-3,3′-pyrrolidine]-5′-carboxamide (71.0 mg, crude) as a yellow solid. LC-MS (ESI, m/z): 250 [M+H]⁺.

### (3R,5′S)-2-oxospiro[indoline-3,3′-pyrrolidine]-5′-carboxamide

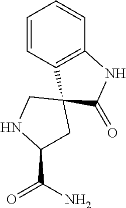 [SMILES: NC(=O)[C@@H]1C[C@@]2(CN1)C(=O)Nc1ccccc12]

1′-(t-butyl) 5′-methyl (3R,5′S)-2-oxospiro[indoline-3,3′-pyrrolidine]-1′,5′-dicarboxylate was prepared as described in Efremov et al., J. Med. Chem. (2012) 55(21): 9069-9088.

A mixture of 1′-t-butyl 5′-methyl (3R,5′S)-2-oxo-1H-spiro[indole-3,3′-pyrrolidine]-1′,5′-dicarboxylate (13.0 g, 37.5 mmol) and NH₃(g) (300 mL, 7M in MeOH) was stirred for 2 d at 50° C. The mixture was concentrated under reduced pressure to afford the crude product. The crude product was purified by C18 column with CH₃CN/Water (0.05% TFA). The fraction was concentrated under reduced pressure to provide t-butyl (3R,5′S)-5′-carbamoyl-2-oxo-1H-spiro[indole-3,3′-pyrrolidine]-1′-carboxylate (1.9 g, 15%) as an off-white solid. ¹H NMR (400 MHz, DMSO-d₆) δ 10.56-10.74 (m, 1H), 7.44-7.60 (m, 1H), 7.21-7.27 (m, 1H), 6.88-7.17 (m, 4H), 4.33-4.51 (m, 1H), 3.44-3.62 (m, 2H), 2.09-2.29 (m, 2H), 1.29-1.50 (m, 9H). LC-MS (ESI, m/z): 332 [M+H]⁺.

To a stirred mixture of t-butyl (3R,5′S)-5′-carbamoyl-2-oxo-1H-spiro[indole-3,3′-pyrrolidine]-1′-carboxylate (900 mg, 2.71 mmol) in 1,4-dioxane (1 mL) was added HCl (10 mL, 4 mL in 1,4-dioxane). The mixture was stirred for 2 h at rt and concentrated under reduced pressure to afford (3R,5′S)-2-oxo-1H-spiro[indole-3,3′-pyrrolidine]-5′-carboxamide (628 mg, crude) as an off-white solid. LC-MS (ESI, m/z): 232 [M+H]⁺.

### Example 1

### Compounds 1A and 1B

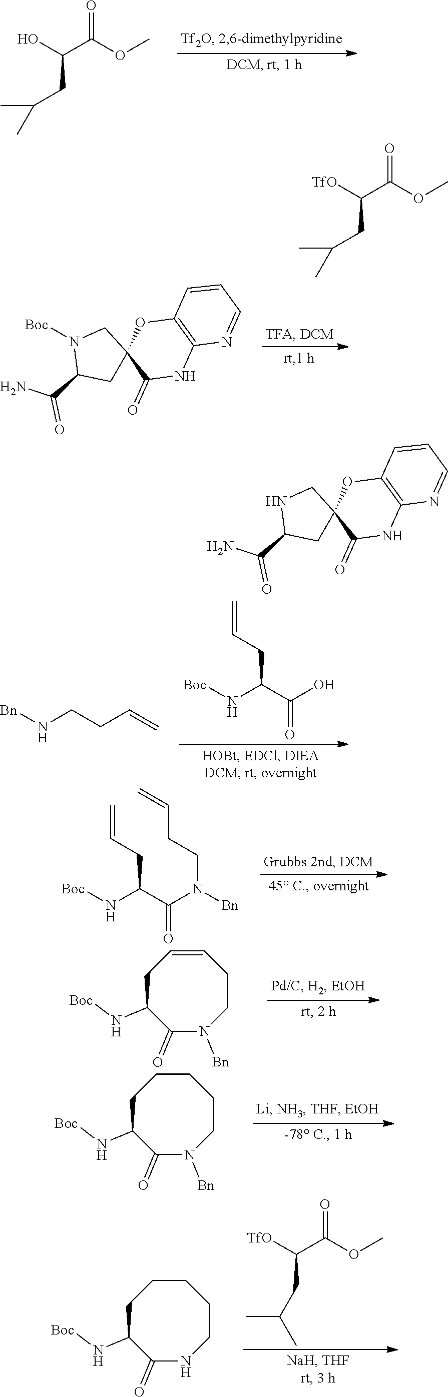

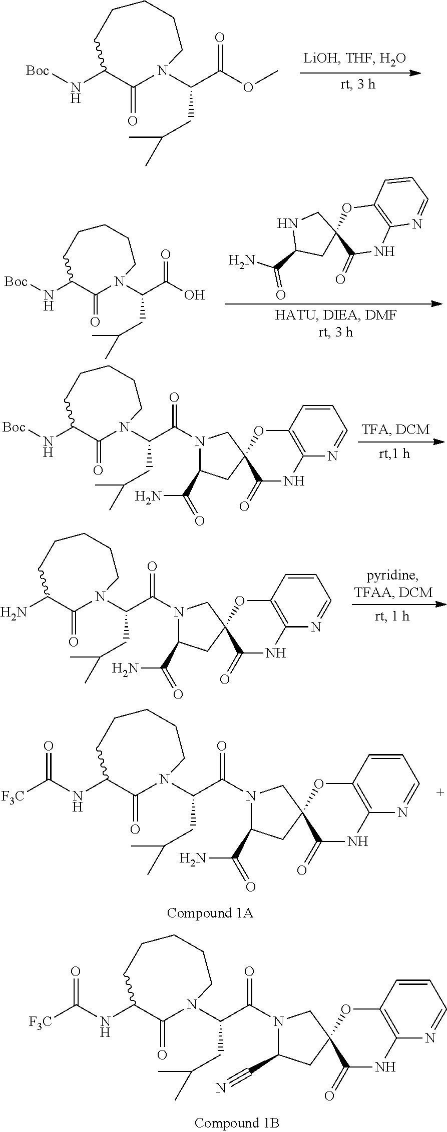

To a solution of methyl (R)-2-hydroxy-4-methylpentanoate (400 mg, 2.73 mmol) in DCM (20 mL) was added 2,6-dimethylpyridine (586 mg, 5.47 mmol) and trifluoromethanesulfonic anhydride (1.39 g, 4.92 mmol) at 0° C. The mixture was stirred for 1 h at room temperature (rt). The reaction was diluted with MTBE (50 mL), washed with brine:1 N HCl (3:1) and dried over anhydrous sodium sulfate. The solids were removed by filtration and the filtrate was concentrated under reduced pressure to afford methyl (R)-4-methyl-2-(((trifluoromethyl)sulfonyl)oxy)pentanoate (758 mg, crude) as a light yellow oil.

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (120 mg, 0.344 mmol) in DCM (3 mL) was added TFA (1 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (85.0 mg, crude) as a yellow solid. LC-MS (ESI, m/z): 249 [M+H]⁺.

To a mixture of N-benzylbut-3-en-1-amine (2.00 g, 12.4 mmol), (S)-2-((t-butoxycarbonyl)amino)pent-4-enoic acid (2.94 g, 13.6 mmol), EDC hydrochloride (2.61 g, 13.6 mmol) and 1-hydroxybenzotriazole (1.68 g, 12.4 mmol) in DCM (50 mL) was added N-ethyl-N-isopropylpropan-2-amine (2.40 g, 18.6 mmol) at 0° C. The mixture was stirred overnight at rt and the reaction was quenched with water (50 mL). The resulting mixture was extracted with DCM (3×50 mL). The organic layers were combined, washed with brine (2×50 mL) and dried over anhydrous sodium sulfate. The solids were removed by filtration and the filtrate was concentrated under reduced pressure to afford the crude product. The crude product was purified by C18 column with CH₃CN/Water (0.05% TFA). The fraction was concentrated under reduced pressure to provide t-butyl (S)-(1-(benzyl(but-3-en-1-yl)amino)-1-oxopent-4-en-2-yl)carbamate (3.70 g, 80%) as a light yellow oil. ¹H NMR (400 MHz, DMSO-d₆) δ 7.16-7.42 (m, 5H), 6.97-7.13 (m, 1H), 5.59-5.85 (m, 2H), 4.92-5.19 (m, 4H), 4.58-4.69 (m, 1H), 4.52-4.57 (m, 1H), 4.37-4.49 (m, 1H), 3.36-3.57 (m, 1H), 3.12-3.29 (m, 1H), 2.17-2.44 (m, 4H), 1.25-1.51 (m, 9H). LC-MS (ESI, m/z): 381 [M+Na]⁺.

To a mixture of t-butyl (S)-(1-(benzyl(but-3-en-1-yl)amino)-1-oxopent-4-en-2-yl)carbamate (3.50 g, 9.76 mmol) in DCM (1 L) was added Grubbs 2nd generation catalyst (1.65 g, 1.95 mmol). The mixture was stirred overnight at 45° C. under nitrogen and the reaction was quenched with water (500 mL). The resulting mixture was extracted with DCM (2×500 mL). The organic layers were combined, washed with brine (2×300 mL) and dried over anhydrous sodium sulfate. The solids were removed by filtration and the filtrate was concentrated under reduced pressure to remove the solvent. The residue was purified by C18 column with CH₃CN/Water (0.05% TFA). The fraction was concentrated under reduced pressure to afford t-butyl (S,Z)-(1-benzyl-2-oxo-1,2,3,4,7,8-hexahydroazocin-3-yl)carbamate (2.0 g crude) as a yellow oil. ¹H NMR (400 MHz, DMSO-d₆) δ 7.20-7.32 (m, 5H), 6.69-6.96 (m, 1H), 5.52-5.69 (m, 1H), 5.35-5.46 (m, 1H), 4.65-4.88 (m, 2H), 4.48-4.61 (m, 1H), 4.04-4.40 (m, 1H), 3.73-3.90 (m, 1H), 3.15-3.52 (m, 1H), 2.59-2.71 (m, 1H), 2.31-2.36 (m, 1H), 2.18-2.27 (m, 1H), 1.35-1.46 (m, 9H). LC-MS (ESI, m/z): 331 [M+H]⁺.

To a mixture of t-butyl (S,Z)-(1-benzyl-2-oxo-1,2,3,4,7,8-hexahydroazocin-3-yl)carbamate (2.00 g, 6.05 mmol) in EtOH (30 mL) was added 10% Palladium on activated carbon (1.20 g). The mixture was stirred for 2 h at rt under hydrogen. The mixture is filtered through packed celite then washed with EtOH (6×30 mL). The filtrate was concentrated under reduced pressure to afford t-butyl (S)-(1-benzyl-2-oxoazocan-3-yl)carbamate (1.90 g, crude) as a brown oil. LC-MS (ESI, m/z): 333 [M+H]⁺.

To a mixture of t-butyl (S)-(1-benzyl-2-oxoazocan-3-yl)carbamate (1.90 g, 5.72 mmol) in THF (20 mL)/EtOH (2 mL)/liquid ammonia (20 mL) was added lithium (400 mg, 57.2 mmol) at −78° C. The mixture was stirred for 1 h at −78° C. and the reaction was quenched with saturated ammonium chloride solution (50 mL). The mixture was extracted with EtOAc (3×50 mL). The organic layers were combined, washed with brine (2×50 mL) and dried over anhydrous sodium sulfate. The solids were removed by filtration and the filtrate was concentrated under reduced pressure to afford the crude product. The crude product was purified by prep-HPLC (Column: XSelect CSH Prep Phenyl OBD C18, 19×250 mm, 5 m; Mobile Phase A: Water (10 mmol/L NH₄HCO₃), Mobile Phase B: ACN; Flow rate: 25 mL/min; Gradient: 35 to 61% B in 15 min; Wave Length: 220 nm; RT1(min): 13.68) to provide t-butyl (S)-(2-oxoazocan-3-yl)carbamate (140 mg, 9%) as a white solid. ¹H NMR (400 MHz, DMSO-d₆) δ 7.41-7.49 (m, 1H), 6.62-6.71 (m, 1H), 4.28-4.40 (m, 1H), 3.34-3.46 (m, 1H), 3.00-3.11 (m, 1H), 1.39-1.73 (m, 8H), 1.31-1.36 (m, 9H). LC-MS (ESI, m/z): 243 [M+H]⁺.

To a solution of t-butyl (S)-(2-oxoazocan-3-yl)carbamate (140 mg, 0.578 mmol) in THF (4 mL) was added sodium hydride (30.0 mg, 0.751 mmol, 60% in oil) at 0° C. After stirring for 1 h at rt, methyl (R)-4-methyl-2-(((trifluoromethyl)sulfonyl)oxy)pentanoate (402 mg, 1.44 mmol) was added. The mixture was stirred for 3 h at rt and the reaction quenched with water (30 mL). The resulting mixture was extracted with EtOAc (3×30 mL). The organic layers were combined, washed with brine (2×30 mL) and dried over anhydrous sodium sulfate. The solids were removed by filtration and the filtrate was concentrated under reduced pressure to remove the solvent. The residue was chromatographed on a silica gel column with EtOAc:PE (1:4) to afford methyl (2S)-2-(3-((t-butoxycarbonyl)amino)-2-oxoazocan-1-yl)-4-methylpentanoate (100 mg, crude) as a yellow oil. LC-MS (ESI, m/z): 371 [M+H]⁺.

To a mixture of methyl (2S)-2-(3-((t-butoxycarbonyl)amino)-2-oxoazocan-1-yl)-4-methylpentanoate (100 mg, 0.270 mmol) in THF (2 mL) was added a solution of LiOH (19.0 mg, 0.810 mmol) in H₂O (2 mL). The mixture was stirred for 3 h at rt and diluted with water (5 mL). The mixture was acidified to pH=6 with HCl (1 M). The resulting mixture was extracted with EtOAc (3×5 mL). The organic layers were combined, washed with brine (2×5 mL) and dried over anhydrous sodium sulfate. The solids were removed by filtration and the filtrate was concentrated under reduced pressure to afford (2S)-2-(3-((t-butoxycarbonyl)amino)-2-oxoazocan-1-yl)-4-methylpentanoic acid (95.0 mg crude) as a yellow semi-solid. LC-MS (ESI, m/z): 379 [M+Na]⁺.

To a mixture of (2R,5′S)-3-oxo-3,4-dihydrospiro[pyrido[3,2-b][1,4]oxazine-2,3′-pyrrolidine]-5′-carboxamide (70.0 mg, 0.282 mmol), (2S)-2-(3-((t-butoxycarbonyl)amino)-2-oxoazocan-1-yl)-4-methylpentanoic acid (91.0 mg, 0.256 mmol) and o-(7-azabenzotriazol-1-yl)-N,N,N′,N′-te-tramethyluroniumhexafluorophosphate (117 mg, 0.308 mmol) in DMF (3 mL) was added N-ethyl-N-isopropylpropan-2-amine (HATU) (166 mg, 1.28 mmol) at −15° C. The mixture was stirred for 3 h at rt and the reaction was quenched with water (10 mL). The resulting mixture was extracted with EtOAc (3×10 mL). The organic layers were combined, washed with brine (2×10 mL) and dried over anhydrous sodium sulfate. The solids were removed by filtration and the filtrate was concentrated under reduced pressure to afford the crude product. The crude product was chromatographed on a silica gel column with MeOH:DCM (5:95) to provide t-butyl (1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (65.0 mg, 39%) as a yellow solid. ¹H NMR (400 MHz, DMSO-d₆) δ 11.49-11.65 (m, 1H), 7.94-8.01 (m, 1H), 7.48 (s, 1H), 7.30-7.40 (m, 1H), 7.16-7.28 (m, 1H), 6.92-7.06 (m, 2H), 5.04-5.45 (m, 1H), 4.43-4.61 (m, 1H), 4.10-4.37 (m, 2H), 3.62-3.98 (m, 1H), 3.34-3.60 (m, 2H), 2.27-2.47 (m, 1H), 2.03-2.22 (m, 1H), 1.45-1.78 (m, 9H), 1.18-1.37 (m, 11H), 0.78-0.91 (m, 6H). LC-MS (ESI, m/z): 587 [M+H]⁺.

A mixture of t-butyl (1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (65.0 mg, 0.111 mmol) in DCM (3 mL) was added TFA (1 mL). The mixture was stirred for 1 h at rt and concentrated under reduced pressure to afford (2R,5′S)-1′-((2S)-2-(3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (53.0 mg, crude) as a yellow semi-solid. LC-MS (ESI, m/z): 487 [M+H]⁺.

To a mixture of (2R,5′S)-1′-((2S)-2-(3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (53.0 mg, 0.109 mmol) in DCM (3 mL) were added pyridine (43.0 mg, 0.545 mmol) and trifluoroacetic anhydride (46.0 mg, 0.218 mmol). The mixture was stirred for 1 h at rt and the reaction was quenched with water (5 mL). The resulting mixture was extracted with DCM (3×5 mL). The organic layers were combined, washed with brine (2×5 mL) and dried over anhydrous sodium sulfate. The solids were removed by filtration and the filtrate was concentrated under reduced pressure to afford the crude product. The crude product was purified by TLC (Mobile phase: EtOAc:PE=1:1; Rf1=0.3; Rf2=0.2; detection: UV) to afford two isomers.

Compound 1A was purified by prep-HPLC (Column: Xbridge Prep OBD C18, 19×250 mm, 5 m; Mobile Phase A: Water (0.1% FA), Mobile Phase B: ACN; Flow rate: 25 mL/min; Gradient: 37 to 67% B in 10 min; Wave Length: 254 nm/220 nm; RT1(min): 8.68) to provide N—((R)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2,2-trifluoroacetamide (Compound 1A, 1.00 mg, 1%) as a white solid. ¹H NMR (400 MHz, 80° C., DMSO-d₆) δ 9.00-9.35 (m, 1H), 7.90-8.02 (m, 1H), 7.22-7.35 (m, 1H), 6.90-7.02 (m, 1H), 5.11-5.25 (m, 1H), 5.00-5.10 (m, 1H), 4.76-4.90 (m, 1H), 3.70-3.90 (m, 1H), 3.60-3.69 (m, 1H), 3.10-3.28 (m, 2H), 2.75-2.85 (m, 2H), 1.88-2.00 (m, 1H), 1.76-1.87 (m, 2H), 1.48-1.75 (m, 6H), 1.28-1.47 (m, 2H), 0.77-1.00 (m, 6H). LC-MS (ESI, m/z): 587 [M+Na]⁺.

Compound 1B was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 28 to 58% B in 10 min; Wave Length: 254 nm/220 nm; Rₜ1(min): 9.27) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2,2-trifluoroacetamide (Compound 1B, 7.50 mg, 12%) as a white solid. ¹H NMR (400 MHz, DMSO-d₆) δ 11.70 (br, 1H), 9.58 (s, 1H), 7.90-8.10 (m, 1H), 7.20-7.33 (m, 1H), 6.95-7.10 (m, 1H), 5.20-5.35 (m, 1H), 4.88-4.97 (m, 1H), 4.70-4.87 (m, 1H), 4.25-4.35 (m, 1H), 4.10-4.24 (m, 1H), 3.48-3.75 (m, 2H), 2.70-2.85 (m, 1H), 2.60-2.69 (m, 1H), 1.30-1.80 (m, 11H), 0.70-1.00 (m, 6H). ¹H NMR (400 MHz, 80° C., DMSO-d₆) δ 11.50 (br, 0.26H), 9.20 (br, 1H), 7.90-8.10 (m, 1H), 7.20-7.35 (m, 1H), 6.95-7.18 (m, 1H), 5.20-5.40 (m, 1H), 4.80-5.10 (m, 2H), 4.15-4.40 (m, 2H), 3.60-3.80 (m, 1H), 3.45-3.59 (m, 1H), 2.72-2.88 (m, 1H), 2.60-2.71 (m, 1H), 1.35-1.90 (m, 11H), 0.70-1.00 (m, 6H). LC-MS (ESI, m/z): 587 [M+Na]⁺.

### Example 2

### Compounds 2A and 2B

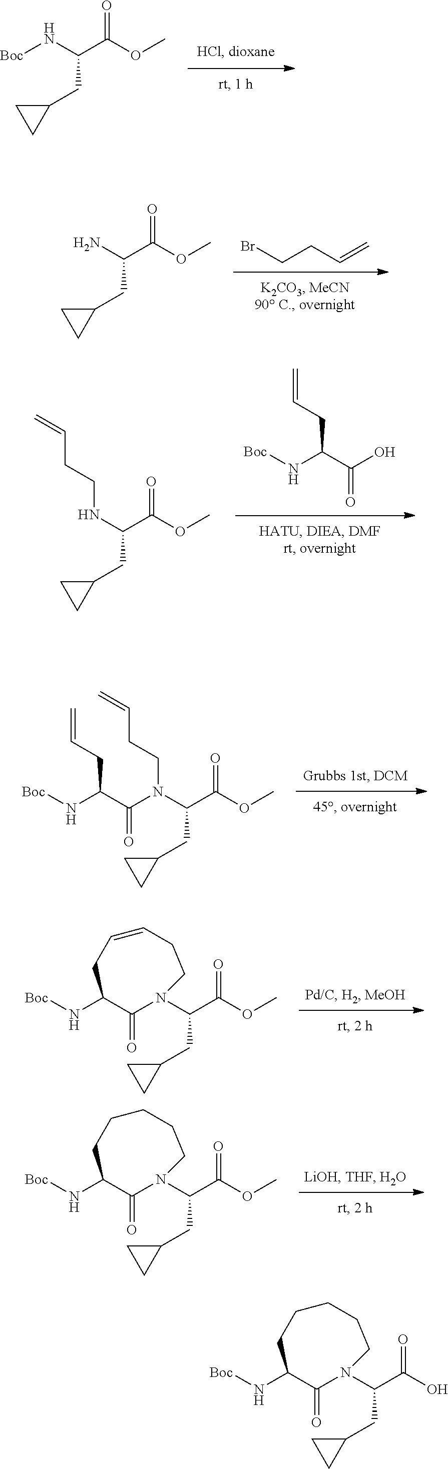

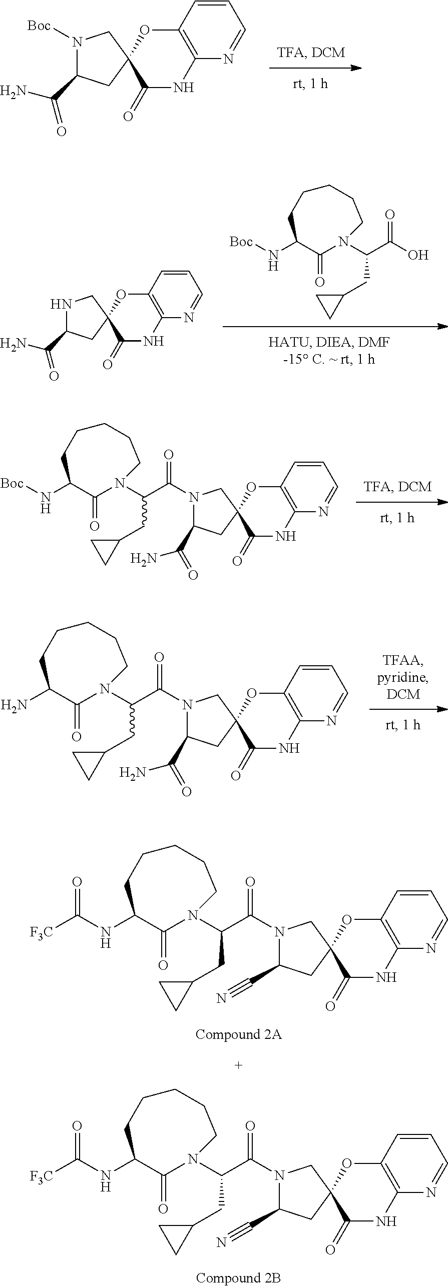

A mixture of methyl (S)-2-((t-butoxycarbonyl)amino)-3-cyclopropylpropanoate (2.00 g, 8.23 mmol) in HCl (40 mL, 4.0 M in 1,4-dioxane) was stirred for 1 h at rt. The mixture was concentrated under reduced pressure to afford methyl (S)-2-amino-3-cyclopropylpropanoate (1.18 g, crude) as an off-white solid. LC-MS (ESI, m/z): 144 [M+H]⁺.

A mixture of methyl (S)-2-amino-3-cyclopropylpropanoate (1.18 g, 8.24 mmol) and potassium carbonate (1.14 g, 8.24 mmol) in MeCN (30 mL) was stirred for 30 mins at rt. 4-bromobut-1-ene (1.11 g, 8.24 mmol) was then added. The mixture was stirred overnight at 90° C. and poured into water (50 mL). The resulting mixture was extracted with EtOAc (3×50 mL). The organic layers were combined, washed with brine (2×50 mL) and dried over anhydrous sodium sulfate. The solids were removed by filtration and the filtrate was concentrated under reduced pressure to afford methyl (S)-2-(but-3-en-1-ylamino)-3-cyclopropylpropanoate (1.00 g, crude) as a yellow oil. LC-MS (ESI, m/z): 198 [M+H]⁺.

To a mixture of methyl (S)-2-(but-3-en-1-ylamino)-3-cyclopropylpropanoate (1.00 g, 5.06 mmol), (S)-2-((t-butoxycarbonyl)amino)pent-4-enoic acid (1.96 g, 9.12 mmol) and HATU (4.43 g, 11.6 mmol) in DMF (25 mL) was added N-ethyl-N-isopropylpropan-2-amine (3.28 g, 25.3 mmol) at 0° C. The mixture was stirred overnight at rt and the reaction was quenched with water (50 mL). The resulting mixture was extracted with EtOAc (3×50 mL). The organic layers were combined, washed with brine (2×50 mL) and dried over anhydrous sodium sulfate. The solids were removed by filtration and the filtrate was concentrated under reduced pressure to afford the crude product. The crude product was chromatographed on a silica gel column with EtOAc:PE (1:4) to provide methyl (S)-2-((S)—N-(but-3-en-1-yl)-2-((t-butoxycarbonyl)amino)pent-4-enamido)-3-cyclopropylpropanoate (1.12 g, 48%) as a light yellow oil. ¹H NMR (400 MHz, DMSO-d₆) δ 6.82-7.20 (m, 1H), 5.61-5.93 (m, 2H), 4.88-5.21 (m, 4H), 4.31-4.55 (m, 1H), 4.14-4.29 (m, 1H), 3.52-3.70 (m, 3H), 3.36-3.50 (m, 2H), 2.22-2.37 (m, 2H), 1.77-1.91 (m, 1H), 1.57-1.71 (m, 1H), 1.31-1.42 (m, 9H), 0.97-1.19 (m, 2H), 0.63-0.73 (m, 1H), 0.31-0.53 (m, 2H), 0.01-0.22 (m, 2H). LC-MS (ESI, m/z): 395 [M+H]⁺.

To a mixture of methyl (S)-2-((S)—N-(but-3-en-1-yl)-2-((t-butoxycarbonyl)amino)pent-4-enamido)-3-cyclopropylpropanoate (1.12 g, 2.83 mmol) in DCM (280 mL) was added benzylidene-bis(tricyclohexylphosphine)dichlororuthenium (281 mg, 0.341 mmol). The mixture was stirred overnight at 45° C. under nitrogen and the reaction was quenched with water (500 mL). The resulting mixture was extracted with DCM (3×200 mL). The organic layers were combined, washed with brine (2×200 mL) and dried over anhydrous sodium sulfate. The solids were removed by filtration and the filtrate was concentrated under reduced pressure to afford the crude product. The crude product was chromatographed on a silica gel column with EtOAc:PE (3:7) to provide methyl (S)-2-((S,Z)-3-((t-butoxycarbonyl)amino)-2-oxo-3,4,7,8-tetrahydroazocin-1(2H)-yl)-3-cyclopropylpropanoate (680 mg, 62%) as a black oil. ¹H NMR (400 MHz, DMSO-d₆) δ 6.57-6.97 (m, 1H), 5.41-5.63 (m, 2H), 4.56-4.83 (m, 2H), 3.65-3.78 (m, 1H), 3.54-3.64 (m, 3H), 3.42-3.53 (m, 1H), 2.53-2.70 (m, 2H), 2.11-2.40 (m, 2H), 1.68-1.82 (m, 1H), 1.45-1.56 (m, 1H), 1.25 (s, 9H), 0.62-0.76 (m, 1H), 0.25-0.45 (m, 2H), 0.01-0.13 (m, 2H). LC-MS (ESI, m/z): 367 [M+H]⁺.

To a mixture of methyl (S)-2-((S,Z)-3-((t-butoxycarbonyl)amino)-2-oxo-3,4,7,8-tetrahydroazocin-1(2H)-yl)-3-cyclopropylpropanoate (170 mg, 0.464 mmol) in MeOH (3 mL) was added 10% palladium on activated carbon (90.0 mg). The mixture was stirred for 2 h at rt under hydrogen. The resulting mixture was filtered through a celite pad and washed with MeOH (10 mL). The filtrate was concentrated under reduced pressure to afford methyl (S)-2-((S)-3-((tert-butoxycarbonyl)amino)-2-oxoazocan-1-yl)-3-cyclopropylpropanoate (160 mg, crude) as a yellow oil. LC-MS (ESI, m/z): 369 [M+H]⁺.

To a mixture of methyl (S)-2-((S)-3-((tert-butoxycarbonyl)amino)-2-oxoazocan-1-yl)-3-cyclopropylpropanoate (160 mg, 0.435 mmol) in THF (2 mL) was added a solution of LiOH (31.0 mg, 1.30 mmol) in H₂O (2 mL). The mixture was stirred for 2 h at rt and diluted with water (5 mL). The mixture was acidified to pH=6 with HCl (1 M). The resulting mixture was extracted with EtOAc (3×5 mL). The organic layers were combined, washed with brine (5 mL) and dried over anhydrous sodium sulfate. The solids were removed by filtration and the filtrate was concentrated under reduced pressure to afford (S)-2-((S)-3-((t-butoxycarbonyl)amino)-2-oxoazocan-1-yl)-3-cyclopropylpropanoic acid (150 mg, crude) as a yellow oil. ¹H NMR (400 MHz, DMSO-d₆) δ 12.49 (br, 1H), 6.51-6.88 (m, 1H), 4.90-5.06 (m, 1H), 4.49-4.81 (m, 1H), 3.61-3.77 (m, 1H), 3.35-3.45 (m, 1H), 1.55-1.73 (m, 7H), 1.41-1.53 (m, 3H), 1.31-1.37 (m, 9H), 0.59-0.73 (m, 1H), 0.24-0.47 (m, 2H), −0.01-0.15 (m, 2H). LC-MS (ESI, m/z): 355 [M+H]⁺.

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (160 mg, 0.459 mmol) in DCM (3 mL) was added TFA (1 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (114 mg, crude) as a yellow semi-solid. LC-MS (ESI, m/z): 249 [M+H]⁺.

To a mixture of (S)-2-((S)-3-((t-butoxycarbonyl)amino)-2-oxoazocan-1-yl)-3-cyclopropylpropanoic acid (163 mg, 0.459 mmol), (2R,5′S)-3-oxo-3,4-dihydrospiro[pyrido[3,2-b][1,4]oxazine-2,3′-pyrrolidine]-5′-carboxamide (114 mg, 0.459 mmol) and HATU (210 mg, 0.552 mmol) in DMF (3 mL) was added N-ethyl-N-isopropylpropan-2-amine (267 mg, 2.06 mmol) at −15° C. The mixture was stirred for 1 h from −15° C. to rt and the reaction was quenched with water (10 mL). The resulting mixture was extracted with EtOAc (3×10 mL). The organic layers were combined, washed with brine (2×10 mL) and dried over anhydrous sodium sulfate. The solids were removed by filtration and the filtrate was concentrated under reduced pressure to afford the crude product. The crude product was purified by TLC (Mobile phase: MeOH:DCM=1:10; Rf=0.4; detection: UV) to provide t-butyl ((3S)-1-(1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (170 mg, 58%) as a yellow solid. ¹H NMR (400 MHz, DMSO-d₆) δ 11.30-11.70 (m, 1H), 7.94-8.02 (m, 1H), 7.40-7.50 (m, 1H), 7.20-7.37 (m, 1H), 6.94-7.12 (m, 2H), 6.76-6.90 (m, 1H), 5.14-5.36 (m, 1H), 4.44-5.08 (m, 2H), 4.08-4.34 (m, 2H), 3.46-3.62 (m, 2H), 2.43-2.49 (m, 1H), 2.10-2.26 (m, 1H), 1.41-1.79 (m, 10H), 1.30-1.36 (m, 9H), 0.58-0.72 (m, 1H), 0.21-0.42 (m, 2H), −0.03-0.08 (m, 2H). LC-MS (ESI, m/z): 607 [M+Na]⁺.

To a mixture of t-butyl ((3S)-1-(1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (85.0 mg, 0.145 mmol) in DCM (3 mL) was added TFA (1 mL). The mixture was stirred for 1 h at rt and concentrated under reduced pressure to afford (2R,5′S)-1′-(2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (70.0 mg, crude) as a yellow solid. LC-MS (ESI, m/z): 485 [M+H]⁺.

To a mixture of (2R,5′S)-1′-(2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (70.0 mg, 0.144 mmol) in DCM (2 mL) were added pyridine (91.0 mg, 1.15 mmol) and trifluoroacetic anhydride (106 mg, 0.504 mmol). The mixture was stirred for 1 h at rt and the reaction was quenched with water (10 mL). The resulting mixture was extracted with DCM (3×10 mL). The organic layers were combined, washed with brine (2×10 mL) and dried over anhydrous sodium sulfate. The solids were removed by filtration and the filtrate was concentrated under reduced pressure to remove the solvent. The residue was purified by TLC (EtOAc:PE=1:1; Rf1=0.5, Rf2=0.4; detection: UV) to afford two isomers.

Compound 2A was purified by prep-Achiral-SFC-HPLC (Column: DAICEL DCpak P4VP 3×25 cm, 5 m; Mobile Phase A: CO₂, Mobile Phase B: MeOH(1%-2M-NH3-MeOH); Flow rate: 65 mL/min; Gradient: isocratic 20% B; Column Temperature (35° C.) Wave Length: 220 nm; Rₜ1(min): 9.78; Sample Solvent: MEOH; Injection Volume: 2.5 mL). N—((S)-1-((R)-1-((2R,5′S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)-2,2,2-trifluoroacetamide (Compound 2A, 2.9 mg, 3%) as a white solid. ¹H NMR (400 MHz, 80° C., DMSO-d₆) δ 11.28 (br, 1H), 9.18 (br, 1H), 7.90-8.00 (m, 1H), 7.15-7.30 (m, 1H), 6.90-7.05 (m, 1H), 5.00-5.30 (m, 2H), 4.75-4.90 (m, 1H), 3.68-3.90 (m, 3H), 3.19-3.40 (m, 1H), 2.70-2.85 (m, 2H), 2.01-2.15 (m, 1H), 1.43-1.90 (m, 8H), 1.12-1.42 (m, 1H), 0.60-0.75 (m, 1H), 0.30-0.55 (m, 2H), 0.10-0.20 (m, 1H), −0.02-0.09 (m, 1H). LC-MS (ESI, m/z): 585 [M+Na]⁺.

Compound 2B was purified by prep-Achiral-SFC-HPLC (Column: DAICEL DCpak P4VP 3×25 cm, 5 m; Mobile Phase A: CO₂, Mobile Phase B: MeOH (1%-2M-NH₃-MeOH); Flow rate: 65 mL/min; Gradient: isocratic 24% B; Column Temperature (35° C.) Wave Length: 220 nm; RT1(min): 8.71; Sample Solvent: MEOH; Injection Volume: 2 mL). N—((S)-1-((S)-1-((2R,5′S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)-2,2,2-trifluoroacetamide (Compound 2B, 11.9 mg, 14%) as a white solid. ¹H NMR (400 MHz, 80° C., DMSO-d₆) δ 11.40 (br, 1H), 9.15 (br, 1H), 7.95-8.05 (m, 1H), 7.20-7.40 (m, 1H), 6.95-7.05 (m, 1H), 5.10-5.30 (m, 1H), 4.78-5.02 (m, 2H), 4.10-4.35 (m, 2H), 3.45-3.80 (m, 2H), 2.65-2.88 (m, 2H), 1.40-1.90 (m, 10H), 0.65-0.72 (m, 1H), 0.28-0.48 (m, 2H), 0.01-0.16 (m, 2H). LC-MS (ESI, m/z): 585 [M+Na]⁺.

### Example 3

### Compounds 3A and 3B

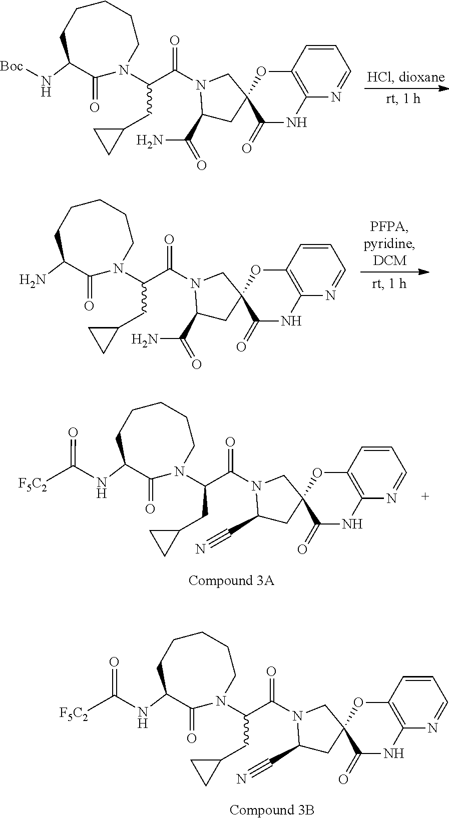

To a mixture of t-butyl ((3S)-1-(1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (270 mg, 0.462 mmol) in DCM (3 mL) was added HCl (6 mL, 4.0 M in 1,4-dioxane). The reaction mixture was stirred for 1 h at rt and concentrated under reduced pressure to afford the crude product (2R,5′S)-1′-(2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (224 mg, crude) as an off-white solid. LC-MS (ESI, m/z): 485[M+H]⁺.

To a mixture of (2R,5′S)-1′-(2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (224 mg, 0.462 mmol) in DCM (5 mL) was added pyridine (292 mg, 3.70 mmol) and 2,2,3,3,3-pentafluoropropanoic anhydride (502 mg, 1.62 mmol). The reaction mixture was stirred for 1 h at rt and quenched with water (10 mL). The resulting mixture was extracted with DCM (3×10 mL) and the organic layers were combined, washed with brine (2×10 mL) and dried over anhydrous sodium sulfate. The solids were removed by filtration and the filtrate was concentrated under reduced pressure to remove the solvent. The residue was purified by TLC (Mobile phase: EtOAc/PE=1:1; Rf=0.4, 0.5; detection: UV) to provide 2 isomers.

Compound 3A was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 31 to 61% B in 10 min; Wave Length: 254 nm/220 nm; RT (min): 9.30) to provide N—((S)-1-((R)-1-((2R,5′S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)-2,2,3,3,3-pentafluoropropanamide (5.20 mg, 1%) as a white solid. ¹H NMR (400 MHz, 80° C., DMSO-d₆) δ 11.10-11.60 (m, 1H), 9.05-9.40 (m, 1H), 7.90-8.10 (m, 1H), 7.25-7.40 (m, 1H), 6.95-7.10 (m, 1H), 5.01-5.70 (m, 2H), 4.77-5.00 (m, 1H), 3.65-4.25 (m, 3H), 3.22-3.38 (m, 1H), 2.73-2.89 (m, 2H), 2.07-2.22 (m, 1H), 1.38-1.90 (m, 8H), 1.20-1.36 (m, 1H), 0.61-0.81 (m, 1H), 0.30-0.50 (m, 2H), 0.01-0.20 (m, 2H). LC-MS (ESI, m/z): 635 [M+Na]⁺.

Compound 3B was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 31 to 61% B in 10 min; Wave Length: 254 nm/220 nm; RT (min): 9.38) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)-2,2,3,3,3-pentafluoropropanamide (36.9 mg, 13%) as a white solid. ¹H NMR (400 MHz, 80° C., DMSO-d₆) δ 11.41 (br, 1H), 9.15 (br, 1H), 7.95-8.03 (m, 1H), 7.25-7.34 (m, 1H), 6.95-7.03 (m, 1H), 5.12-5.28 (m, 1H), 4.81-5.00 (m, 2H), 4.24 (s, 2H), 3.58-3.75 (m, 1H), 3.50-3.57 (m, 1H), 2.76-2.85 (m, 1H), 2.65-2.75 (m, 1H), 1.66-1.89 (m, 3H), 1.41-1.65 (m, 7H), 0.52-0.68 (m, 1H), 0.36-0.42 (m, 1H), 0.25-0.35 (m, 1H), 0.03-0.13 (m, 2H). LC-MS (ESI, m/z): 635 [M+Na]⁺.

### Example 4

### Compounds 4A and 4B

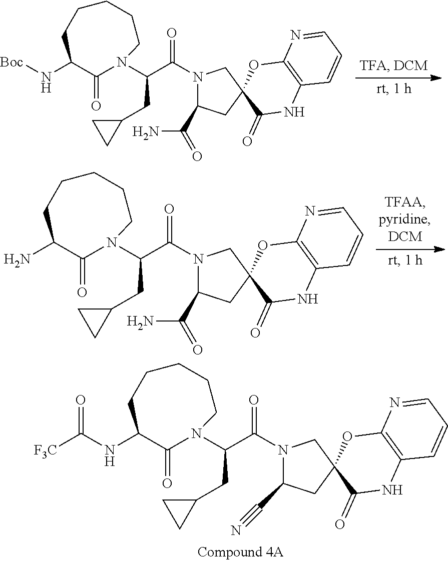

To a mixture of t-butyl ((S)-1-((R)-1-((3R,5′S)-5′-carbamoyl-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)carbamate (50.0 mg, 0.086 mmol) in DCM (1.5 mL) was added TFA (0.5 mL). The mixture was stirred for 1 h at rt and concentrated under reduced pressure to afford (3R,5′S)-1′-((R)-2-((S)-3-amino-2-oxoazocan-1-yl)-3-cyclopropylpropanoyl)-2-oxo-1,2-dihydrospiro[pyrido[2,3-b][1,4]oxazine-3,3′-pyrrolidine]-5′-carboxamide (41.0 mg, crude) as a yellow oil. LC-MS (ESI, m/z): 485 [M+H]⁺.

To a mixture of (3R,5′S)-1′-((R)-2-((S)-3-amino-2-oxoazocan-1-yl)-3-cyclopropylpropanoyl)-2-oxo-1,2-dihydrospiro[pyrido[2,3-b][1,4]oxazine-3,3′-pyrrolidine]-5′-carboxamide (41.0 mg, 0.085 mmol) in DCM (1 mL) were added pyridine (33.0 mg, 0.425 mmol) and trifluoroacetic anhydride (35.0 mg, 0.170 mmol). The mixture was stirred for 1 h at rt and the reaction was quenched with water (5 mL). The resulting mixture was extracted with DCM (3×5 mL). The organic layers were combined, washed with brine (2×5 mL) and dried over anhydrous sodium sulfate. The solids were removed by filtration, and the filtrate was concentrated under reduced pressure to remove the solvent. The residue was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 21 to 51% B in 10 min; Wave Length: 254 nm/220 nm; RT1(min): 9.3). The product was further purified by prep-SFC-HPLC (Column: CHIRAL ART Cellulose-SC, 2×25 cm, 5 m; Mobile Phase A: Hex(0.1% FA), Mobile Phase B: EtOH:DCM=1:1; Flow rate: 20 mL/min; Gradient: isocratic 20% B; Wave Length: 254/220 nm; RT1(min): 11.585). Purification resulted in N—((S)-1-((R)-1-((3R,5′S)-5′-cyano-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)-2,2,2-trifluoroacetamide (5.80 mg, 12%) as a white solid. ¹H NMR (400 MHz, 80° C., DMSO-d₆) δ 10.99 (br, 1H), 9.19 (br, 1H), 7.75-7.90 (m, 1H), 7.20-7.40 (m, 1H), 7.00-7.19 (m, 1H), 4.99-5.40 (m, 2H), 4.70-4.98 (m, 1H), 3.60-3.92 (m, 3H), 3.20-3.40 (m, 1H), 2.81-2.95 (m, 2H), 2.01-2.21 (m, 1H), 1.71-1.98 (m, 2H), 1.55-1.70 (m, 4H), 1.35-1.54 (m, 2H), 1.16-1.34 (m, 1H), 0.60-0.78 (m, 1H), 0.43-0.53 (m, 1H), 0.31-0.42 (m, 1H), 0.11-0.20 (m, 1H), 0.02-0.10 (m, 1H). LC-MS (ESI, m/z): 563 [M+H]⁺.

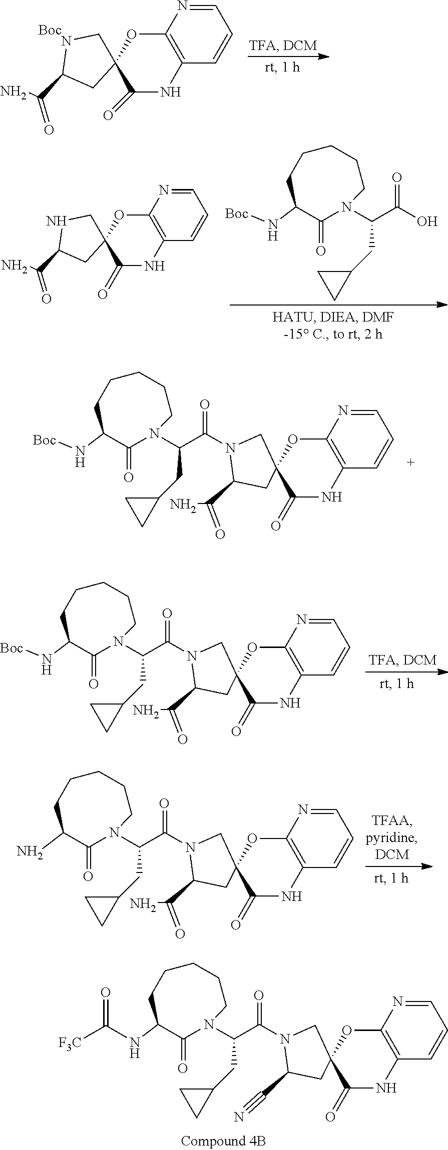

To a mixture of t-butyl (3R,5′S)-5′-carbamoyl-2-oxo-1,2-dihydrospiro[pyrido[2,3-b][1,4]oxazine-3,3′-pyrrolidine]-1′-carboxylate (90.0 mg, 0.258 mmol) in DCM (3 mL) was added TFA (1 mL). The mixture was stirred for 1 h at rt and concentrated under reduced pressure to afford (3R,5′S)-2-oxo-1,2-dihydrospiro[pyrido[2,3-b][1,4]oxazine-3,3′-pyrrolidine]-5′-carboxamide (64.0 mg, crude) as a yellow solid. LC-MS (ESI, m/z): 249 [M+H]⁺.

To a mixture of (3R,5′S)-2-oxo-1,2-dihydrospiro[pyrido[2,3-b][1,4]oxazine-3,3′-pyrrolidine]-5′-carboxamide (64.0 mg, 0.258 mmol), (S)-2-((S)-3-((t-butoxycarbonyl)amino)-2-oxoazocan-1-yl)-3-cyclopropylpropanoic acid (91.0 mg, 0.258 mmol) and HATU (118 mg, 0.310 mmol) in DMF (3 mL) was added N-ethyl-N-isopropylpropan-2-amine (150 mg, 1.16 mmol) at −15° C. The mixture was stirred for 1 h from −15° C. to rt and the reaction was quenched with water (10 mL). The resulting mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:10; Rf1=0.5, Rf1=0.4; detection: UV) to provide two isomers.

Isomer 1: t-butyl ((S)-1-((R)-1-((3R,5′S)-5′-carbamoyl-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)carbamate (40.0 mg, 26%) as a light yellow solid. ¹H NMR (400 MHz, DMSO-d₆) δ 11.00 (s, 1H), 7.81-7.88 (m, 1H), 7.47-7.55 (m, 1H), 7.21-7.28 (m, 1H), 7.05-7.13 (m, 2H), 6.81-6.88 (m, 1H), 4.99-5.11 (m, 1H), 4.51-4.59 (m, 1H), 4.39-4.49 (m, 1H), 3.69-3.76 (m, 1H), 3.45-3.55 (m, 2H), 3.15-3.21 (m, 1H), 2.53-2.57 (m, 1H), 2.44-2.49 (m, 1H), 2.17-2.24 (m, 1H), 1.62-1.69 (m, 2H), 1.45-1.55 (m, 6H), 1.30-1.37 (m, 9H), 0.80-0.87 (m, 1H), 0.62-0.69 (m, 1H), 0.34-0.43 (m, 2H), 0.06-0.12 (m, 1H), −0.06-0.00 (m, 1H). LC-MS (ESI, m/z): 585 [M+H]⁺.

Isomer 2: t-butyl ((S)-1-((S)-1-((3R,5′S)-5′-carbamoyl-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)carbamate (70.0 mg, 43%) as a light yellow solid. ¹H NMR (400 MHz, DMSO-d₆) δ 11.19 (s, 1H), 7.82-7.89 (m, 1H), 7.51 (s, 1H), 7.27-7.33 (m, 1H), 7.06-7.12 (m, 1H), 6.98 (s, 1H), 6.79-6.86 (m, 1H), 5.26-5.35 (m, 1H), 4.49-4.60 (m, 1H), 4.29-4.39 (m, 1H), 4.20-4.28 (m, 1H), 4.05-4.16 (m, 1H), 3.46-3.64 (m, 2H), 2.42-2.49 (m, 1H), 2.13-2.24 (m, 1H), 1.38-1.76 (m, 10H), 1.28-1.34 (m, 9H), 0.60-0.70 (m, 1H), 0.19-0.38 (m, 2H), 0.01-0.10 (m, 2H). LC-MS (ESI, m/z): 585 [M+H]⁺.

To a mixture of t-butyl ((S)-1-((S)-1-((3R,5′S)-5′-carbamoyl-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)carbamate (70.0 mg, 0.120 mmol) in DCM (3 mL) was added TFA (1 mL). The mixture was stirred for 1 h at rt and concentrated under reduced pressure to afford (3R,5′S)-1′-((S)-2-((S)-3-amino-2-oxoazocan-1-yl)-3-cyclopropylpropanoyl)-2-oxo-1,2-dihydrospiro[pyrido[2,3-b][1,4]oxazine-3,3′-pyrrolidine]-5′-carboxamide (58.0 mg, crude) as a yellow oil. LC-MS (ESI, m/z): 485 [M+H]⁺.

To a mixture of (3R,5′S)-1′-((S)-2-((S)-3-amino-2-oxoazocan-1-yl)-3-cyclopropylpropanoyl)-2-oxo-1,2-dihydrospiro[pyrido[2,3-b][1,4]oxazine-3,3′-pyrrolidine]-5′-carboxamide (58.0 mg, 0.120 mmol) in DCM (2 mL) were added pyridine (47.0 mg, 0.600 mmol) and trifluoroacetic anhydride (50.0 mg, 0.240 mmol). The mixture was stirred for 1 h at rt and the reaction was quenched with water (5 mL). The resulting mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: Xbridge Prep OBD C18, 19×250 mm, 5 m; Mobile Phase A: Water (10 mmol/L NH₄HCO₃), Mobile Phase B: MEOH; Flow rate: 25 mL/min; Gradient: 35 to 60% B in 15 min; Wave Length: 254 nm/220 nm; RT1(min): 12.33) to provide N—((S)-1-((S)-1-((3R,5′S)-5′-cyano-2-oxo-1,2-dihydrospiro[pyrido[2,3-b][1,4]oxazine-3,3′-pyrrolidin]-1′-yl)-3-cyclopropyl-1-oxopropan-2-yl)-2-oxoazocan-3-yl)-2,2,2-trifluoroacetamide (26.3 mg, 39%) as a white solid. ¹H NMR (400 MHz, 80° C., DMSO-d₆) δ 9.18 (br, 1H), 7.80-7.89 (m, 1H), 7.28-7.38 (m, 1H), 7.02-7.11 (m, 1H), 5.15-5.30 (m, 1H), 4.80-5.00 (m, 2H), 4.21-4.38 (m, 2H), 3.50-3.75 (m, 2H), 2.68-2.88 (m, 2H), 1.40-1.85 (m, 10H), 0.50-0.69 (m, 1H), 0.28-0.48 (m, 2H), 0.02-0.12 (m, 2H). LC-MS (ESI, m/z): 563 [M+H]⁺.

### Example 5

### Compound 5

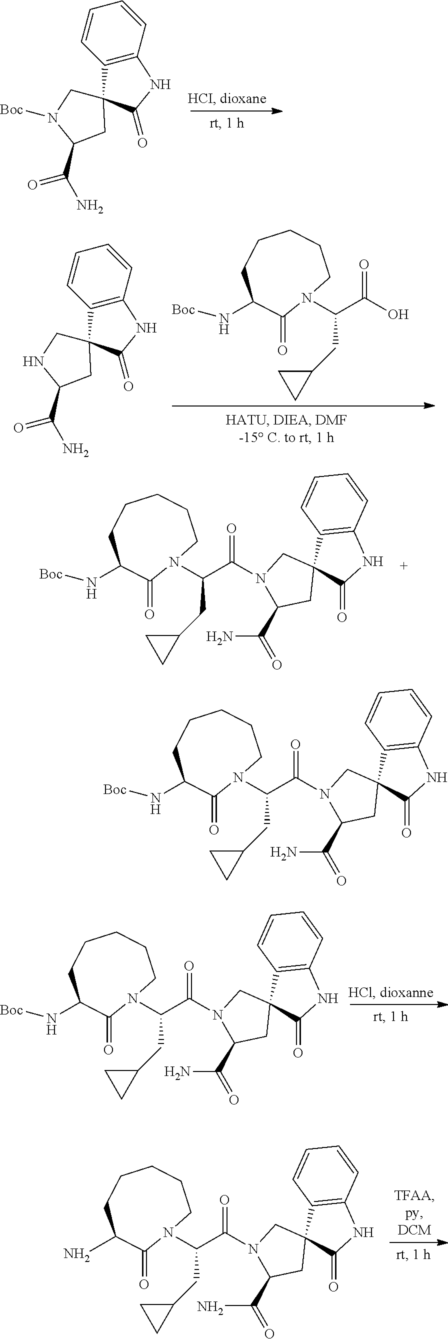

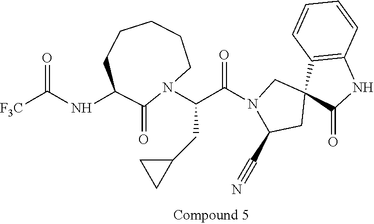 [SMILES: N#C[C@@H]1C[C@@]2(CN1C(=O)[C@H](CC1CC1)N1CCCCC[C@H](NC(=O)C(F)(F)F)C1=O)C(=O)Nc1ccccc12]

A mixture of t-butyl (3R,5′S)-5′-carbamoyl-2-oxospiro[indoline-3,3′-pyrrolidine]-1′-carboxylate (450 mg, 1.36 mmol) in HCl (6 mL, 4 M in 1,4-dioxane) was stirred for 1 h at rt and concentrated under reduced pressure to afford (3R,5′S)-2-oxospiro[indoline-3,3′-pyrrolidine]-5′-carboxamide (313 mg, crude) as an off-white solid. LC-MS (ESI, m/z): 232 [M+H]⁺.

To a mixture of (3R,5′S)-2-oxospiro[indoline-3,3′-pyrrolidine]-5′-carboxamide (313 mg, 1.36 mmol), (S)-2-((S)-3-((t-butoxycarbonyl)amino)-2-oxoazocan-1-yl)-3-cyclopropylpropanoic acid (479 mg, 1.35 mmol) and HATU (616 mg, 1.62 mmol) in DMF (3 mL) was added N-ethyl-N-isopropylpropan-2-amine (523 mg, 4.05 mmol) at −15° C. The mixture was stirred for 1 h at rt and the reaction was quenched with water (10 mL). The resulting mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:12; Rf=0.5, 0.4; detection: UV) to provide the isomer 1 and isomer 2.

Isomer 1: t-butyl ((S)-1-((R)-1-((3R,5′S)-5′-carbamoyl-2-oxospiro[indoline-3,3′-pyrrolidin]-1′-yl)-3-cyclopropyl-1-oxopropan-2-yl)-2-oxoazocan-3-yl)carbamate (160 mg, 20%). ¹H NMR (400 MHz, DMSO-d₆) δ 10.54 (s, 1H), 7.48 (s, 1H), 7.19-7.28 (m, 1H), 7.10 (s, 1H), 6.92-6.99 (m, 1H), 6.87-6.91 (m, 1H), 6.80-6.86 (m, 2H), 5.00-5.10 (m, 1H), 4.65-4.74 (m, 1H), 4.42-4.52 (m, 1H), 3.64-3.78 (m, 2H), 3.39-3.48 (m, 1H), 3.19-3.26 (m, 1H), 2.15-2.29 (m, 2H), 1.47-1.70 (m, 6H), 1.21-1.46 (m, 12H), 0.67-0.81 (m, 2H), 0.34-0.44 (m, 1H), 0.22-0.32 (m, 1H), 0.07-0.18 (m, 1H), −0.12-−0.12 (m, 1H). LC-MS (ESI, m/z): 568 [M+H]⁺.

Isomer 2: t-butyl ((S)-1-((S)-1-((3R,5′S)-5′-carbamoyl-2-oxospiro[indoline-3,3′-pyrrolidin]-1′-yl)-3-cyclopropyl-1-oxopropan-2-yl)-2-oxoazocan-3-yl)carbamate (410 mg, 53%) as an off-white solid. ¹H NMR (400 MHz, DMSO-d₆) δ 10.74 (s, 1H), 7.53 (s, 1H), 7.19-7.28 (m, 1H), 7.03 (s, 1H), 6.88-7.01 (m, 3H), 6.75 (d, J=7.6 Hz, 1H), 5.24-5.34 (m, 1H), 4.55-4.63 (m, 1H), 4.44-4.54 (m, 1H), 3.86-3.95 (m, 1H), 3.75-3.84 (m, 1H), 3.50-3.61 (m, 2H), 2.17-2.27 (m, 2H), 1.57-1.69 (m, 2H), 1.37-1.56 (m, 4H), 1.24-1.36 (m, 11H), 1.10-1.21 (m, 1H), 0.53-0.76 (m, 2H), 0.19-0.42 (m, 2H), −0.02-0.08 (m, 2H). LC-MS (ESI, m/z): 568 [M+H]⁺.

A mixture of t-butyl ((S)-1-((S)-1-((3R,5′S)-5′-carbamoyl-2-oxospiro[indoline-3,3′-pyrrolidin]-1′-yl)-3-cyclopropyl-1-oxopropan-2-yl)-2-oxoazocan-3-yl)carbamate (90.0 mg, 0.159 mmol) in HCl (2 mL, 4 M in 1,4-dioxane) was stirred for 1 h at rt and concentrated under reduced pressure to afford (3R,5′S)-1′-((S)-2-((S)-3-amino-2-oxoazocan-1-yl)-3-cyclopropylpropanoyl)-2-oxospiro[indoline-3,3′-pyrrolidine]-5′-carboxamide (74.0 mg, crude) as an off-white solid. LC-MS (ESI, m/z): 468 [M+H]⁺.

To a solution of (3R,5′S)-1′-((S)-2-((S)-3-amino-2-oxoazocan-1-yl)-3-cyclopropylpropanoyl)-2-oxospiro[indoline-3,3′-pyrrolidine]-5′-carboxamide (74.0 mg, 0.159 mmol) in DCM (2 mL) were added pyridine (63.0 mg, 0.795 mmol) and TFAA (670 mg, 0.318 mmol). The mixture was stirred for 1 h at rt and the reaction was quenched with water (5 mL). The resulting mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: XSelect CSH Prep Fluoro-Phenyl, 30×150 mm, 5 m; Mobile Phase A: Water (0.1% FA), Mobile Phase B: ACN; Flow rate: 60 mL/min; Gradient: 22 to 52% B in 9 min; Wave Length: 254 nm/220 nm; RT1(min): 8.8) to provide N—((S)-1-((S)-1-((3R,5′S)-5′-cyano-2-oxospiro[indoline-3,3′-pyrrolidin]-1′-yl)-3-cyclopropyl-1-oxopropan-2-yl)-2-oxoazocan-3-yl)-2,2,2-trifluoroacetamide (33.1 mg, 38%) as a white solid. ¹H NMR (400 MHz, 80° C., DMSO-d₆) δ 10.50 (br, 1H), 9.12 (br, 1H), 7.20-7.30 (m, 1H), 7.08-7.19 (m, 1H), 6.88-7.01 (m, 2H), 5.01-5.30 (m, 2H), 4.75-4.92 (m, 1H), 3.90-4.10 (m, 2H), 3.50-3.78 (m, 2H), 2.58-2.70 (m, 1H), 2.51-2.57 (m, 1H), 1.48-1.80 (m, 9H), 1.12-1.36 (m, 1H), 0.52-0.70 (m, 1H), 0.36-0.48 (m, 2H), 0.05-0.19 (m, 2H). LC-MS (ESI, m/z): 544 [M−H]—.

### Example 6

### Compound 6

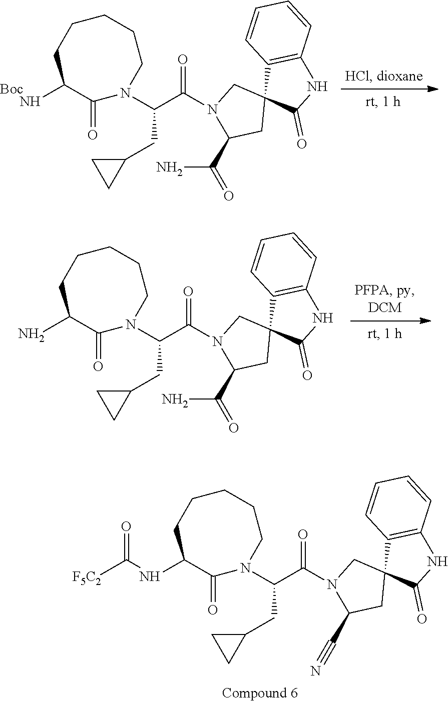

A mixture of t-butyl ((S)-1-((S)-1-((3R,5′S)-5′-carbamoyl-2-oxospiro[indoline-3,3′-pyrrolidin]-1′-yl)-3-cyclopropyl-1-oxopropan-2-yl)-2-oxoazocan-3-yl)carbamate (100 mg, 0.176 mmol) in HCl (2 mL, 4 M in 1,4-dioxane) was stirred for 1 h at rt and concentrated under reduced pressure to afford (3R,5′S)-1′-((S)-2-((S)-3-amino-2-oxoazocan-1-yl)-3-cyclopropylpropanoyl)-2-oxospiro[indoline-3,3′-pyrrolidine]-5′-carboxamide (82.0 mg, crude) as an off-white solid. LC-MS (ESI, m/z): 468 [M+H]⁺.

To a solution of (3R,5′S)-1′-((S)-2-((S)-3-amino-2-oxoazocan-1-yl)-3-cyclopropylpropanoyl)-2-oxospiro[indoline-3,3′-pyrrolidine]-5′-carboxamide (82.0 mg, 0.176 mmol) in DCM (2 mL) were added pyridine (70.0 mg, 0.880 mmol) and perfluoropropionic anhydride (109 mg, 0.352 mmol). The mixture was stirred 1 h at rt and the reaction was quenched with water (5 mL). The resulting mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 32 to 62% B in 10 min; Wave Length: 254 nm/220 nm; RT1(min): 9.4) to provide N—((S)-1-((S)-1-((3R,5′S)-5′-cyano-2-oxospiro[indoline-3,3′-pyrrolidin]-1′-yl)-3-cyclopropyl-1-oxopropan-2-yl)-2-oxoazocan-3-yl)-2,2,3,3,3-pentafluoropropanamide (37.0 mg, 35%) as a white solid. ¹H NMR (400 MHz, 60° C., DMSO-d₆) δ 10.60 (br, 1H), 9.34 (br, 1H), 7.20-7.40 (m, 1H), 7.05-7.19 (m, 1H), 6.80-7.04 (m, 2H), 5.09-5.40 (m, 2H), 4.70-5.05 (m, 1H), 3.90-4.20 (m, 2H), 3.51-3.86 (m, 2H), 2.55-2.72 (m, 2H), 1.39-1.90 (m, 9H), 1.11-1.33 (m, 1H), 0.55-0.75 (m, 1H), 0.21-0.48 (m, 2H), 0.02-0.19 (m, 2H). LC-MS (ESI, m/z): 618 [M+Na]⁺.

### Example 7

### Compound 7

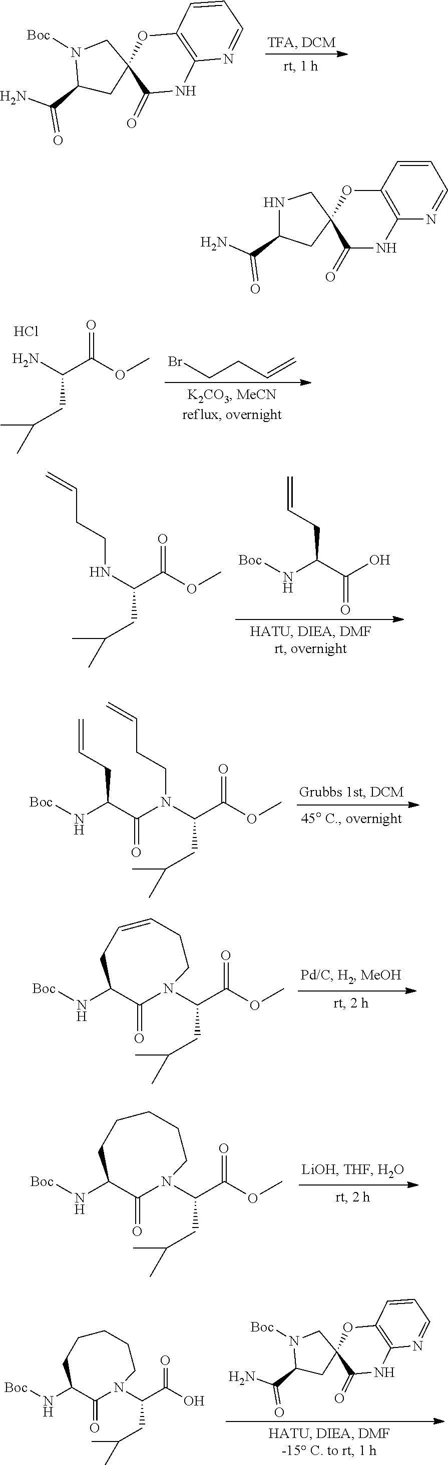

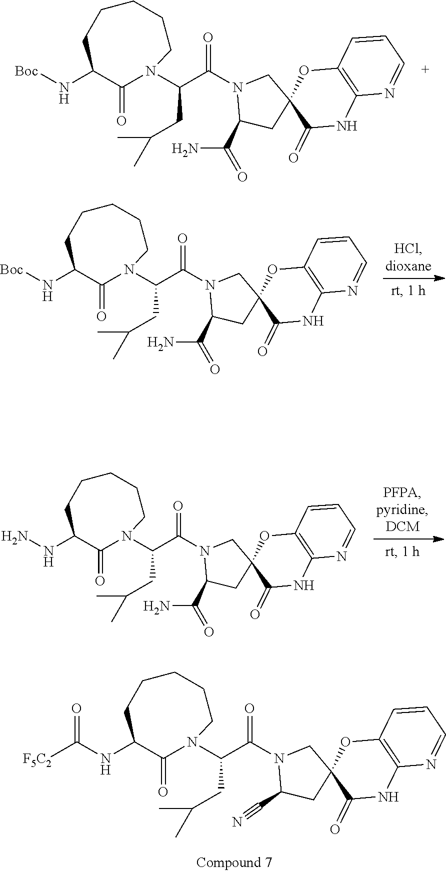

To a mixture of methyl L-leucinate hydrochloride (5.00 g, 27.5 mmol) in MeCN (150 mL) was added potassium carbonate (4.18 g, 30.2 mmol). After stirring for 30 mins at rt, 4-bromobut-1-ene (4.09 g, 30.2 mmol) was added and refluxed overnight. The mixture was filtered through a celite pad and washed with EtOAc. The filtrate was quenched with water (200 mL). The resulting mixture was extracted with EtOAc (3×200 mL). The organic layers were combined, washed with brine (2×300 mL), dried over anhydrous sodium sulfate, filtered and concentrated under reduced pressure to afford methyl but-3-en-1-yl-L-leucinate (2.85 g, crude) as a yellow oil. LC-MS (ESI, m/z): 200 [M+H]⁺.

To a mixture of methyl but-3-en-1-yl-L-leucinate (2.85 g, 14.3 mmol), (S)-2-((t-butoxycarbonyl)amino)pent-4-enoic acid (5.54 g, 25.7 mmol) and HATU (12.5 g, 32.8 mmol) in DMF (40 mL) was added N-ethyl-N-isopropylpropan-2-amine (9.24 g, 71.5 mmol) at 0° C. The mixture was stirred overnight at rt and the reaction was quenched with water (100 mL). The resulting mixture was extracted with EtOAc (3×100 mL). The organic layers were combined, washed with brine (2×100 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl N-(but-3-en-1-yl)-N—((S)-2-((tert-butoxycarbonyl)amino)pent-4-enoyl)-L-leucinate (2.26 g, 37%) as a brown oil. ¹H NMR (400 MHz, DMSO-d₆) δ 6.93-7.26 (m, 1H), 5.70-5.89 (m, 2H), 4.97-5.18 (m, 4H), 4.50-4.66 (m, 1H), 4.26-4.41 (m, 1H), 3.79-3.87 (m, 2H), 3.55-3.70 (m, 3H), 3.34-3.44 (m, 1H), 2.21-2.38 (m, 3H), 1.63-1.76 (m, 2H), 1.46-1.58 (m, 1H), 1.31-1.43 (m, 9H), 0.80-1.01 (m, 6H). LC-MS (ESI, m/z): 397 [M+H]⁺.

To a mixture of methyl N-(but-3-en-1-yl)-N—((S)-2-((tert-butoxycarbonyl)amino)pent-4-enoyl)-L-leucinate (2.10 g, 5.30 mmol) in DCM (530 mL, 0.01 M) was added Grubbs 1st (539 mg, 0.636 mmol). The mixture was stirred overnight at 45° C. and the reaction was quenched with water (300 mL). The resulting mixture was extracted with DCM (2×300 mL). The organic layers were combined, washed with brine (2×500 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl (S)-2-((S,Z)-3-((tert-butoxycarbonyl)amino)-2-oxo-3,4,7,8-tetrahydroazocin-1(2H)-yl)-4-methylpentanoate (1.45 g, 70%) as a black oil. ¹H NMR (400 MHz, DMSO-d₆) δ 6.60-7.06 (m, 1H), 5.31-5.68 (m, 2H), 4.91-5.12 (m, 1H), 4.56-4.76 (m, 1H), 3.98-4.11 (m, 1H), 3.49-3.73 (m, 4H), 3.30-3.48 (m, 1H), 2.08-2.40 (m, 2H), 1.72-1.87 (m, 1H), 1.60-1.71 (m, 1H), 1.47-1.60 (m, 2H), 1.31-1.44 (m, 9H), 0.73-1.03 (m, 6H). LC-MS (ESI, m/z): 369 [M+H]⁺.

To a mixture of methyl (S)-2-((S,Z)-3-((tert-butoxycarbonyl)amino)-2-oxo-3,4,7,8-tetrahydroazocin-1(2H)-yl)-4-methylpentanoate (1.45 g, 3.93 mmol) in MeOH (20 mL) was added 10% palladium on activated carbon (750 mg). The mixture was stirred for 2 h at rt under hydrogen. The mixture was filtered through a celite pad and washed with MeOH. The filtrate was concentrated under reduced pressure to afford methyl (S)-2-((S)-3-((tert-butoxycarbonyl)amino)-2-oxoazocan-1-yl)-4-methylpentanoate (1.35 g, 87%, crude) as a yellow oil. ¹H NMR (400 MHz, DMSO-d₆) δ 6.59-6.97 (m, 1H), 5.04-5.19 (m, 1H), 4.47-4.61 (m, 1H), 3.59-3.63 (m, 3H), 3.13-3.42 (m, 2H), 1.42-1.94 (m, 11H), 1.29-1.41 (m, 9H), 0.67-1.02 (m, 6H). LC-MS (ESI, m/z): 371 [M+H]⁺.

To a mixture of methyl (S)-2-((S)-3-((tert-butoxycarbonyl)amino)-2-oxoazocan-1-yl)-4-methylpentanoate (1.35 g, 3.51 mmol) in THF (15 mL) was added a solution of LiOH (252 mg, 10.5 mmol) in H₂O (15 mL). The mixture was stirred for 2 h at rt and diluted with water (20 mL). The mixture was acidified to pH=5 with HCl (1 M). The resulting mixture was extracted with EtOAc (3×20 mL). The organic layers were combined, washed with brine (20 mL) and dried over anhydrous sodium sulfate. The solids were removed by filtration and the filtrate was concentrated under reduced pressure to afford (S)-2-((S)-3-((tert-butoxycarbonyl)amino)-2-oxoazocan-1-yl)-4-methylpentanoic acid (720 mg, 52%, crude) as a brown oil. ¹H NMR (400 MHz, DMSO-d₆) δ 6.55-6.93 (m, 1H), 4.93-5.13 (m, 1H), 4.45-4.63 (m, 1H), 3.92-4.12 (m, 1H), 3.22-3.44 (m, 2H), 1.44-1.86 (m, 11H), 1.29-1.42 (m, 9H), 0.70-0.97 (m, 6H). LC-MS (ESI, m/z): 357 [M+H]⁺.

To a mixture of tert-butyl (2R,5′S)-5′-carbamoyl-3-oxo-3,4-dihydrospiro[pyrido[3,2-b][1,4]oxazine-2,3′-pyrrolidine]-1′-carboxylate (150 mg, 0.431 mmol) in DCM (3 mL) was added TFA (1 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (107 mg, crude) as a yellow solid. LC-MS (ESI, m/z): 249 [M+H]⁺.

To a mixture of (S)-2-((S)-3-((tert-butoxycarbonyl)amino)-2-oxoazocan-1-yl)-4-methylpentanoic acid (150 mg, 0.421 mmol), (2R,5′S)-3-oxo-3,4-dihydrospiro[pyrido[3,2-b][1,4]oxazine-2,3′-pyrrolidine]-5′-carboxamide (104 mg, 0.421 mmol) and HATU (240 mg, 0.631 mmol) in DMF (5 mL) was added N-ethyl-N-isopropylpropan-2-amine (408 mg, 3.15 mmol) at −15° C. The mixture was stirred for 1 h from −15° C. to rt and the reaction was quenched with water (10 mL). The resulting mixture was extracted with EtOAc (3×10 mL). The organic layers were combined, washed with brine (2×20 mL) and dried over anhydrous sodium sulfate. The solids were removed by filtration and the filtrate was concentrated under reduced pressure to afford the crude product. The crude product was purified by TLC (Mobile phase: MeOH:DCM=1:10) to afford t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (100 mg, 38%) as a light yellow solid. ¹H NMR (400 MHz, DMSO-d₆) δ 11.62 (s, 1H), 7.93-8.05 (m, 1H), 7.49 (s, 1H), 7.19-7.27 (m, 1H), 6.96-7.09 (m, 2H), 6.81-6.95 (m, 1H), 5.28-5.41 (m, 1H), 4.45-4.58 (m, 1H), 4.10-4.34 (m, 3H), 3.46-3.67 (m, 2H), 2.33-2.49 (m, 1H), 2.08-2.20 (m, 1H), 1.64-1.76 (m, 1H), 1.39-1.63 (m, 8H), 1.28-1.38 (m, 10H), 1.19-1.27 (m, 1H), 0.76-0.94 (m, 6H). LC-MS (ESI, m/z): 609 [M+Na]⁺.

A mixture of tert-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (100 mg, 0.170 mmol) in HCl (3 mL, 4.0 M in 1,4-dioxane) was stirred for 1 h at rt and concentrated under reduced pressure to afford (2R,5′S)-1′-((S)-2-((S)-3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (83.0 mg, crude) as an off-white solid. LC-MS (ESI, m/z): 487 [M+H]⁺.

To a mixture of (2R,5′S)-1′-((S)-2-((S)-3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (83.0 mg, 0.171 mmol) in DCM (3 mL) were added pyridine (67.0 mg, 0.855 mmol) and perfluoropropionic anhydride (105 mg, 0.342 mmol). The mixture was stirred for 1 h at rt and the reaction was quenched with water (5 mL). The resulting mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient (B %): 33 to 63% B in 10 min; Wave Length: 254 nm/220 nm; RT1(min): 9.7) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2,3,3,3-pentafluoropropanamide (33.9 mg, 32%) as a white solid. ¹H NMR (400 MHz, 80° C., DMSO-d₆) δ 11.42 (br, 1H), 9.26 (s, 1H), 7.88-8.09 (m, 1H), 7.10-7.32 (m, 1H), 6.81-7.09 (m, 1H), 5.20-5.38 (m, 1H), 4.78-5.01 (m, 2H), 4.16-4.37 (m, 2H), 3.61-3.72 (m, 1H), 3.47-3.60 (m, 1H), 2.71-2.85 (m, 1H), 2.58-2.70 (m, 1H), 1.71-1.78 (m, 2H), 1.51-1.70 (m, 7H), 1.33-1.50 (m, 2H), 0.83-0.98 (m, 3H), 0.71-0.82 (m, 3H). LC-MS (ESI, m/z): 671 [M+H]⁺.

### Example 8

### Compound 8

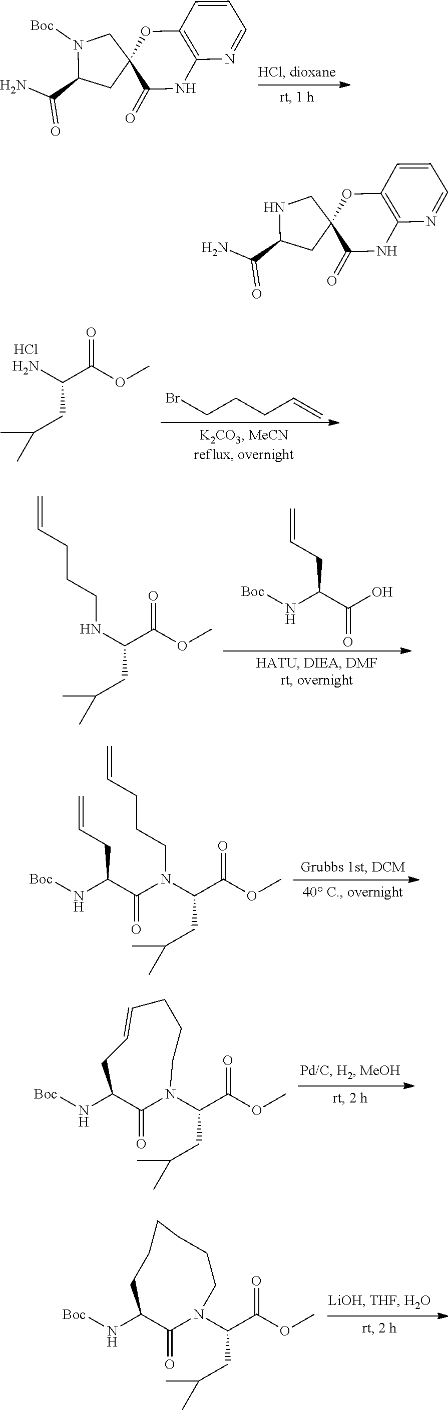

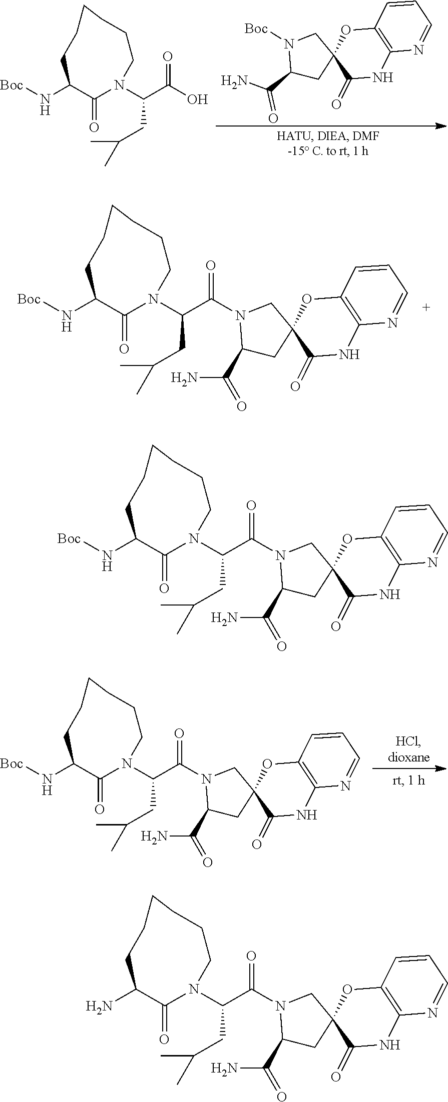

A mixture of t-butyl (2R,5′S)-5′-carbamoyl-3-oxo-3,4-dihydrospiro[pyrido[3,2-b][1,4]oxazine-2,3′-pyrrolidine]-1′-carboxylate (150 mg, 0.431 mmol) in HCl (5 mL, 4 M in 1,4-dioxane) was stirred for 1 h at rt. The mixture was concentrated under reduced pressure to afford (2R,5′S)-3-oxo-3,4-dihydrospiro[pyrido[3,2-b][1,4]oxazine-2,3′-pyrrolidine]-5′-carboxamide (106 mg, crude) as a light brown solid. LC-MS (ESI, m/z): 249 [M+H]⁺.

A mixture of methyl L-leucinate hydrochloride (5 g, 27.5 mmol) and potassium carbonate (4.18 g, 30.2 mmol) in MeCN (150 mL) was stirred for 30 mins at rt. 5-bromopent-1-ene (4.51 g, 30.2 mmol) then added. The mixture was refluxed overnight. The mixture was filtered and the filtrate was quenched with water (200 mL). The resulting mixture was extracted with EtOAc (3×150 mL). The organic layers were combined, washed with brine (2×100 mL), dried over anhydrous sodium sulfate, filtered and concentrated under reduced pressure to afford methyl pent-4-en-1-yl-L-leucinate (3.0 g, crude) as a yellow liquid. LC-MS (ESI, m/z): 214 [M+H]⁺.

To a mixture of methyl pent-4-en-1-yl-L-leucinate (3.00 g, 14.0 mmol), (S)-2-((t-butoxycarbonyl)amino)pent-4-enoic acid (5.45 g, 25.3 mmol) and HATU (12.3 g, 32.3 mmol) in DMF (40 mL) was added N-ethyl-N-isopropylpropan-2-amine (9.09 g, 70.3 mmol) at 0° C. The mixture was stirred overnight at rt and the reaction was quenched with water (100 mL). The mixture was extracted with EtOAc (3×100 mL). The organic layers were combined, washed with brine (2×100 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl N—((S)-2-((t-butoxycarbonyl)amino)pent-4-enoyl)-N-(pent-4-en-1-yl)-L-leucinate (2.4 g, 37%) as a brown oil. ¹H NMR (400 MHz, DMSO-d₆) δ 6.79-7.03 (m, 1H), 5.61-5.93 (m, 2H), 4.88-5.21 (m, 4H), 4.49-4.67 (m, 1H), 4.24-4.40 (m, 1H), 3.52-3.63 (m, 3H), 3.13-3.43 (m, 2H), 2.17-2.39 (m, 2H), 1.91-2.13 (m, 2H), 1.46-1.86 (m, 5H), 1.26-1.44 (m, 9H), 0.73-1.02 (m, 6H). LC-MS (ESI, m/z): 411 [M+H]⁺.

To a mixture of methyl N—((S)-2-((t-butoxycarbonyl)amino)pent-4-enoyl)-N-(pent-4-en-1-yl)-L-leucinate (2.40 g, 5.84 mmol) in DCM (580 mL, 0.01 M) was added Grubbs 1st (747 mg, 0.877 mmol) at rt. The mixture was stirred overnight at 40° C. and the reaction was quenched with water (500 mL). The mixture was extracted with DCM (2×500 mL). The organic layers were combined, washed with brine (2×500 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl (S)-2-((S,E)-3-((t-butoxycarbonyl)amino)-2-oxo-2,3,4,7,8,9-hexahydro-1H-azonin-1-yl)-4-methylpentanoate (1.50 g, 60%) as a black oil. ¹H NMR (400 MHz, DMSO-d₆) δ 6.70-7.01 (m, 1H), 5.65-5.76 (m, 1H), 5.45-5.62 (m, 1H), 4.31-4.72 (m, 2H), 3.51-3.65 (m, 4H), 3.15-3.36 (m, 1H), 2.16-2.40 (m, 2H), 1.55-1.80 (m, 4H), 1.23-1.48 (m, 12H), 0.79-0.94 (m, 6H). LC-MS (ESI, m/z): 383 [M+H]⁺.

To a mixture of methyl (S)-2-((S,E)-3-((t-butoxycarbonyl)amino)-2-oxo-2,3,4,7,8,9-hexahydro-1H-azonin-1-yl)-4-methylpentanoate (1.50 g, 3.89 mmol) in MeOH (30 mL) was added 10% palladium on activated carbon (800 mg). The mixture was stirred for 2 h at rt under hydrogen. The mixture was filtered through a celite pad and washed with MeOH. The filtrate was concentrated under reduced pressure to afford methyl (S)-2-((S)-3-((t-butoxycarbonyl)amino)-2-oxoazonan-1-yl)-4-methylpentanoate (1.35 g, 72%, crude) as a brown oil. ¹H NMR (400 MHz, DMSO-d₆) δ 6.57-7.12 (m, 1H), 5.20-5.30 (m, 1H), 4.56-4.71 (m, 1H), 3.61-3.72 (m, 4H), 3.30-3.42 (m, 1H), 1.33-1.80 (m, 20H), 1.21-1.27 (m, 2H), 0.75-0.92 (m, 6H). LC-MS (ESI, m/z): 385 [M+H]⁺.

To a mixture of methyl (S)-2-((S)-3-((t-butoxycarbonyl)amino)-2-oxoazonan-1-yl)-4-methylpentanoate (1.35 g, 3.51 mmol) in THF (15 mL) was added a solution of LiOH (0.25 g, 10.5 mmol) in water (15 mL). The mixture was stirred for 2 h at rt and diluted with water (20 mL). The mixture was adjusted to pH=6 with HCl (1 M). The mixture was extracted with EtOAc (3×50 mL). The organic layers were combined, washed with brine (30 mL), dried over anhydrous sodium sulfate, filtered and concentrated under reduced pressure to provide (S)-2-((S)-3-((t-butoxycarbonyl)amino)-2-oxoazonan-1-yl)-4-methylpentanoic acid (1.20 g, 78%) as a light brown semi-solid. ¹H NMR (400 MHz, DMSO-d₆) δ 12.65 (br, 1H), 6.51-7.11 (m, 1H), 5.13-5.28 (m, 1H), 4.53-4.76 (m, 1H), 3.34-3.42 (m, 2H), 1.72-1.95 (m, 5H), 1.34-1.50 (m, 15H), 1.19-1.28 (m, 2H), 0.76-0.89 (m, 6H). LC-MS (ESI, m/z): 371 [M+H]⁺.

To a mixture of (2R,5′S)-3-oxo-3,4-dihydrospiro[pyrido[3,2-b][1,4]oxazine-2,3′-pyrrolidine]-5′-carboxamide (106 mg, 0.427 mmol), (S)-2-((S)-3-((t-butoxycarbonyl)amino)-2-oxoazonan-1-yl)-4-methylpentanoic acid (159 mg, 0.427 mmol) and HATU (195 mg, 0.512 mmol) in DMF (5 mL) was added N-ethyl-N-isopropylpropan-2-amine (249 mg, 1.92 mmol) at −15° C. The mixture was stirred for 1 h from −15° C. to rt and the reaction was quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM) to afford t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazonan-3-yl)carbamate (150 mg, 55%) as a light yellow solid. ¹H NMR (400 MHz, DMSO-d₆) δ 11.63 (s, 1H), 7.94-8.03 (m, 1H), 7.45-7.52 (m, 1H), 6.94-7.28 (m, 4H), 5.34-5.44 (m, 1H), 4.54-4.73 (m, 1H), 4.26-4.37 (m, 2H), 4.01-4.15 (m, 1H), 3.54-3.66 (m, 2H), 2.41-2.49 (m, 1H), 1.98-2.08 (m, 1H), 1.26-1.75 (m, 22H), 0.76-0.90 (m, 6H). LC-MS (ESI, m/z): 623 [M+Na]⁺.

A mixture of t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-methyl-1-oxopentan-2-yl)-2-oxoazonan-3-yl)carbamate (150 mg, 0.250 mmol) in HCl (10 mL, 4 M in 1,4-dioxane) was stirred for 1 h at rt. The mixture was concentrated under reduced pressure to afford (2R,5′S)-1′-((S)-2-((S)-3-amino-2-oxoazonan-1-yl)-4-methylpentanoyl)-3-oxo-3,4-dihydrospiro[pyrido[3,2-b][1,4]oxazine-2,3′-pyrrolidine]-5′-carboxamide (125 mg, crude) as a light yellow solid. LC-MS (ESI, m/z): 501 [M+H]⁺.

To a mixture of (2R,5′S)-1′-((S)-2-((S)-3-amino-2-oxoazonan-1-yl)-4-methylpentanoyl)-3-oxo-3,4-dihydrospiro[pyrido[3,2-b][1,4]oxazine-2,3′-pyrrolidine]-5′-carboxamide (125 mg, 0.250 mmol) in DCM (4 mL) were added pyridine (99.0 mg, 1.25 mmol) and perfluoropropionic anhydride (155 mg, 0.500 mmol). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient (B %): 35 to 65% B in 10 min; Wave Length: 254 nm/220 nm; RT1(min): 9.5) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazonan-3-yl)-2,2,3,3,3-pentafluoropropanamide (9.0 mg, 5%) as a white solid. ¹H NMR (400 MHz, 100° C., DMSO-d₆) δ 11.40 (br, 0.4H), 9.28 (br, 1H), 7.98-8.05 (m, 1H), 7.20-7.30 (m, 1H), 6.95-7.10 (m, 1H), 5.30-5.50 (m, 1H), 4.90-5.10 (m, 2H), 4.26-4.40 (m, 1H), 4.15-4.25 (m, 1H), 3.60-3.80 (m, 2H), 2.75-2.85 (m, 1H), 2.58-2.68 (m, 1H), 1.80-2.02 (m, 3H), 1.45-1.75 (m, 9H), 1.20-1.39 (m, 1H), 0.75-1.00 (m, 6H). LC-MS (ESI, m/z): 651 [M+Na]⁺.

### Example 9

### Compound 9

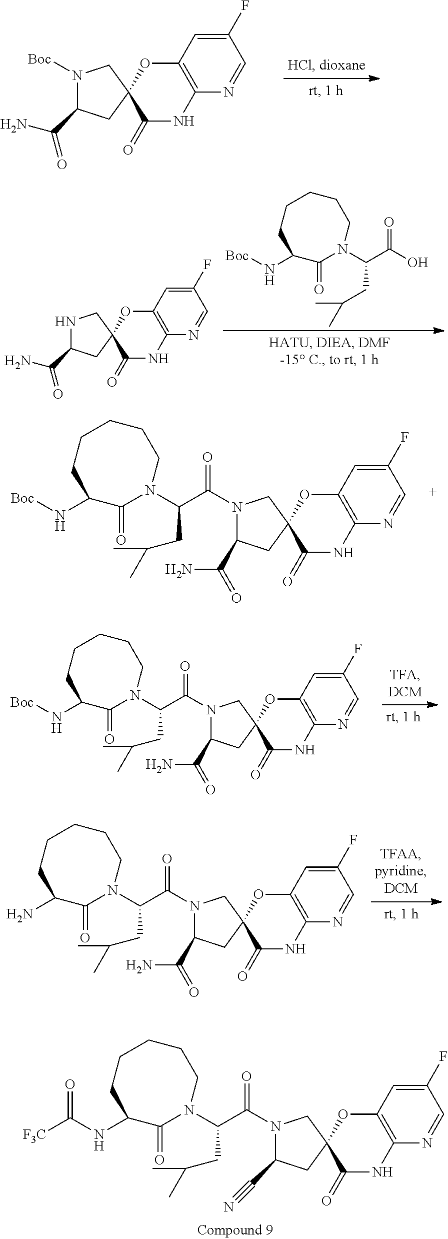

A mixture of tert-butyl (2R,5′S)-5′-carbamoyl-7-fluoro-3-oxo-3,4-dihydrospiro[pyrido[3,2-b][1,4]oxazine-2,3′-pyrrolidine]-1′-carboxylate (150 mg, 0.409 mmol) in HCl (5 mL, 4.0 M in dioxane) was stirred for 1 h at rt. The mixture was concentrated under reduced pressure to afford (2R,5′S)-7-fluoro-3-oxo-3,4-dihydrospiro[pyrido[3,2-b][1,4]oxazine-2,3′-pyrrolidine]-5′-carboxamide (110 mg, crude) as an off-white solid. LC-MS (ESI, m/z): 267 [M+H]⁺.

To a mixture of (S)-2-((S)-3-((tert-butoxycarbonyl)amino)-2-oxoazocan-1-yl)-4-methylpentanoic acid (147 mg, 0.412 mmol), (2R,5′S)-7-fluoro-3-oxo-3,4-dihydrospiro[pyrido[3,2-b][1,4]oxazine-2,3′-pyrrolidine]-5′-carboxamide (110 mg, 0.412 mmol) and HATU (188 mg, 0.494 mmol) in DMF (5 mL) was added N-ethyl-N-isopropylpropan-2-amine (160 mg, 1.24 mmol) at −15° C. The mixture was stirred for 1 h from −15° C. to rt and the reaction was quenched with water (10 mL). The mixture was extracted with EA (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:12; Rf1=0.4; Rf2=0.3, detection: UV) to provide two isomers.

Isomer 1: t-butyl ((S)-1-((R)-1-((2R,5′S)-5′-carbamoyl-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (60 mg, 24%) as a light brown solid. ¹H NMR (400 MHz, DMSO-d₆) δ 11.42-11.70 (m, 1H), 7.98-8.07 (m, 1H), 7.46-7.56 (m, 1H), 7.41 (s, 1H), 7.06-7.18 (m, 1H), 6.87-7.03 (m, 1H), 5.08-5.30 (m, 1H), 4.40-4.68 (m, 2H), 3.82-4.28 (m, 1H), 3.58-3.73 (m, 1H), 3.51-3.57 (m, 1H), 3.28-3.39 (m, 1H), 2.52-2.65 (m, 1H), 2.31-2.46 (m, 1H), 1.82-1.98 (m, 1H), 1.40-1.81 (m, 8H), 1.22-1.39 (m, 10H), 1.10-1.21 (m, 1H), 0.74-0.98 (m, 6H). LC-MS (ESI, m/z): 605 [M+H]⁺.

Isomer 2: t-butyl ((S)-1-((S)-1-((2R,5′S)-5′-carbamoyl-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (100 mg, 40%) as a light brown solid. ¹H NMR (400 MHz, DMSO-d₆) δ 11.74 (s, 1H), 8.00-8.10 (m, 1H), 7.49 (s, 1H), 7.24-7.35 (m, 1H), 7.00 (s, 1H), 6.58-6.93 (m, 1H), 5.26-5.35 (m, 1H), 4.44-4.56 (m, 1H), 4.26-4.36 (m, 1H), 4.02-4.24 (m, 2H), 3.40-3.61 (m, 2H), 2.52-2.59 (m, 1H), 2.11-2.29 (m, 1H), 1.39-1.76 (m, 9H), 1.28-1.38 (m, 10H), 1.20-1.27 (m, 1H), 0.74-0.92 (m, 6H). LC-MS (ESI, m/z): 605 [M+H]⁺.

To a mixture of tert-butyl ((S)-1-((S)-1-((2R,5′S)-5′-carbamoyl-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (100 mg, 0.165 mmol) in DCM (3 mL) was added TFA (1 mL). The mixture was stirred for 1 h at rt and concentrated under reduced pressure to afford (2R,5′S)-1′-((S)-2-((S)-3-amino-2-oxoazocan-1-yl)-4-methylpentanoyl)-7-fluoro-3-oxo-3,4-dihydrospiro[pyrido[3,2-b][1,4]oxazine-2,3′-pyrrolidine]-5′-carboxamide (83.0 mg, crude) as a brown semi-solid. LC-MS (ESI, m/z): 505 [M+H]⁺.

To a mixture of (2R,5′S)-1′-((S)-2-((S)-3-amino-2-oxoazocan-1-yl)-4-methylpentanoyl)-7-fluoro-3-oxo-3,4-dihydrospiro[pyrido[3,2-b][1,4]oxazine-2,3′-pyrrolidine]-5′-carboxamide (83.0 mg, 0.164 mmol) in DCM (3 mL) were added pyridine (91.0 mg, 1.15 mmol) and trifluoroacetic anhydride (121 mg, 0.574 mmol). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to remove the solvent. The residue was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 30 to 60% B in 10 min; Wave Length: 254 nm/220 nm; RT1(min): 9.6) to afford the crude product (mixture of ~3% isomer). The product was further separated by prep-chiral-HPLC-column (Column: CHIRALPAK IG, 2×25 cm, 5 m; Mobile Phase A: Hex(0.1% FA)-HPLC, Mobile Phase B: EtOH:DCM=1:1; Flow rate: 20 mL/min; Gradient (B %): isocratic 40; Wave Length: 220 nm. Purification resulted in N—((S)-1-((S)-1-((2R,5′S)-5′-cyano-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2,2-trifluoroacetamide (33.7 mg, 35%) as a white solid. ¹H NMR (400 MHz, 80° C., DMSO-d₆) δ 11.58 (s, 1H), 9.10-9.30 (m, 1H), 8.00-8.10 (m, 1H), 7.22-7.35 (m, 1H), 5.20-5.32 (m, 1H), 4.90-5.00 (m, 1H), 4.75-4.89 (m, 1H), 4.20-4.35 (m, 2H), 3.61-3.76 (m, 1H), 3.50-3.60 (m, 1H), 2.78-2.90 (m, 1H), 2.60-2.77 (m, 1H), 1.40-1.85 m, 11H), 0.88-0.99 (m, 3H), 0.75-0.87 (m, 3H). LC-MS (ESI, m/z): 605 [M+Na]⁺.

### Example 10

### Compound 10

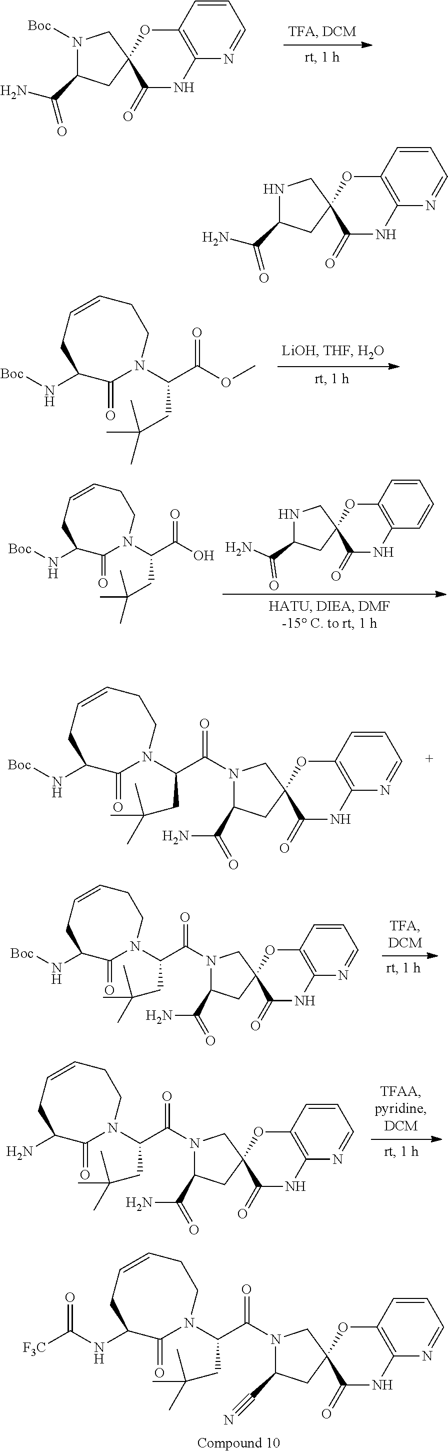

To a mixture of methyl (S)-2-((S,Z)-3-((tert-butoxycarbonyl)amino)-2-oxo-3,4,7,8-tetrahydroazocin-1(2H)-yl)-4,4-dimethylpentanoate (200 mg, 0.523 mmol) in THF (2 mL) was added a solution of LiOH (37.0 mg, 1.56 mmol) in H₂O (2 mL). The mixture was stirred for 1 h at rt and the reaction was quenched with water (5 mL). The mixture was acidified to pH=6 with HCl (aq., 1 M). The mixture was extracted with EtOAc (3×5 mL). The organic layers were combined, washed with brine (2×5 mL), dried over anhydrous sodium sulfate, filtered and concentrated under reduced pressure to afford (S)-2-((S,Z)-3-((tert-butoxycarbonyl)amino)-2-oxo-3,4,7,8-tetrahydr oazocin-1(2H)-yl)-4,4-dimethylpentanoic acid (180 mg, 88%, crude) as a brown semi-solid. ¹H NMR (400 MHz, DMSO-d₆) δ 12.45 (br, 1H), 6.67-6.89 (m, 1H), 5.39-5.60 (m, 2H), 4.90-5.03 (m, 1H), 4.60-4.77 (m, 1H), 3.44-3.73 (m, 2H), 2.55-2.70 (m, 1H), 2.26-2.41 (m, 1H), 2.09-2.23 (m, 1H), 1.70-1.83 (m, 1H), 1.46-1.55 (m, 1H), 1.31-1.44 (m, 9H), 1.15-1.28 (m, 1H), 0.71-0.96 (m, 9H). LC-MS (ESI, m/z): 369 [M+H]⁺.

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (160 mg, 0.459 mmol) in DCM (3 mL) was added TFA (1 mL). The mixture was stirred for 1 h at rt and concentrated under reduced pressure to afford (S)-2-((S,Z)-3-((tert-butoxycarbonyl)amino)-2-oxo-3,4,7,8-tetrahydroazocin-1(2H)-yl)-4,4-dimethylpentanoic acid (114 mg, crude) as a yellow semi-solid. LC-MS (ESI, m/z): 271 [M+Na]⁺.

To a mixture of (S)-2-((S,Z)-3-((tert-butoxycarbonyl)amino)-2-oxo-3,4,7,8-tetrahydroazocin-1(2H)-yl)-4,4-dimethylpentanoic acid (170 mg, 0.461 mmol), (S)-2-((S,Z)-3-((tert-butoxycarbonyl)amino)-2-oxo-3,4,7,8-tetrahydroazocin-1(2H)-yl)-4,4-dimethylpentanoic acid (114 mg, 0.461 mmol) and HATU (210 mg, 0.553 mmol) in DMF (5 mL) was added N-ethyl-N-isopropylpropan-2-amine (268 mg, 2.07 mmol) at −15° C. The mixture was stirred for 1 h from −15° C. to rt and the reaction was quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:10; Rf 1=0.4, Rf 2=0.3; detection: UV) to provide two isomers.

Isomer 1: (2R,5′S)-1′-((S)-2-((S,Z)-3-amino-2-oxo-3,4,7,8-tetrahydroazocin-1(2H)-yl)-4,4-dimethylpentanoyl)-3-oxo-3,4-dihydrospiro[pyrido[3,2-b][1,4]oxazine-2,3′-pyrrolidine]-5′-carboxamide (30.0 mg, 8%) as a light yellow solid. ¹H NMR (400 MHz, DMSO-d₆) δ 11.32-11.72 (m, 1H), 7.85-8.05 (m, 1H), 7.34-7.60 (m, 1H), 7.16-7.28 (m, 1H), 6.98-7.13 (m, 2H), 6.79-6.97 (m, 1H), 5.32-5.72 (m, 2H), 4.96-5.13 (m, 1H), 4.54-4.77 (m, 1H), 4.33-4.52 (m, 1H), 3.64-3.80 (m, 1H), 3.47-3.63 (m, 1H), 3.35-3.44 (m, 1H), 3.10-3.25 (m, 1H), 2.38-2.48 (m, 1H), 2.00-2.36 (m, 4H), 1.19-1.38 (m, 10H), 1.00-1.14 (m, 1H), 0.90-0.98 (m, 1H), 0.71-0.89 (m, 9H). LC-MS (ESI, m/z): 621 [M+Na]⁺.

Isomer 2: tert-butyl ((S,Z)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxo-1,2,3,4,7,8-hexahydroazocin-3-yl)carbamate (160 mg, 55%) as a light yellow solid. ¹H NMR (400 MHz, DMSO-d₆) δ 11.62 (s, 1H), 7.92-8.07 (m, 1H), 7.44 (s, 1H), 7.19-7.29 (m, 1H), 7.02-7.13 (m, 1H), 6.96 (s, 1H), 6.74-6.89 (m, 1H), 5.59-5.79 (m, 1H), 5.47-5.58 (m, 1H), 5.29-5.40 (m, 1H), 4.49-4.69 (m, 1H), 4.07-4.33 (m, 3H), 3.61-3.78 (m, 1H), 3.49-3.60 (m, 1H), 2.38-2.49 (m, 2H), 2.18-2.30 (m, 1H), 2.01-2.16 (m, 2H), 1.65-1.77 (m, 1H), 1.31-1.52 (m, 9H), 1.21-1.30 (m, 2H), 0.68-1.03 (m, 9H). LC-MS (ESI, m/z): 621 [M+Na]⁺.

To a mixture of tert-butyl ((S,Z)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxo-1,2,3,4,7,8-hexahydroazocin-3-yl)carbamate (160 mg, 0.267 mmol) in DCM (3 mL) was added TFA (1 mL). The mixture was stirred for 1 h at rt and concentrated under reduced pressure to afford (2R,5′S)-1′-((S)-2-((S,Z)-3-amino-2-oxo-3,4,7,8-tetrahydroazocin-1(2H)-yl)-4,4-dimethylpentanoyl)-3-oxo-3,4-dihydrospiro[pyrido[3,2-b][1,4]oxazine-2,3′-pyrrolidine]-5′-carboxamide (133 mg, crude) as a yellow solid. LC-MS (ESI, m/z): 499 [M+H]⁺.

To a mixture of (2R,5′S)-1′-((S)-2-((S,Z)-3-amino-2-oxo-3,4,7,8-tetrahydroazocin-1(2H)-yl)-4,4-dimethylpentanoyl)-3-oxo-3,4-dihydrospiro[pyrido[3,2-b][1,4]oxazine-2,3′-pyrrolidine]-5′-carboxamide (133 mg, 0.267 mmol) in DCM (3 mL) was added pyridine (105 mg, 1.33 mmol) and trifluoroacetic anhydride (112 mg, 0.534 mmol). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient (B %): 29 to 59% B in 10 min; Wave Length: 254 nm/220 nm; RT1(min): 9.8) to provide the crude product (mixture of ~5% isomer); the crude product separated by prep-Chiral-HPLC (Column: CHIRALPAK ID, 2×25 cm, 5 m; Mobile Phase A: Hex(0.1% FA), Mobile Phase B: MeOH:DCM=1:1; Flow rate: 20 mL/min; Gradient (B %): isocratic 45; Wave Length: 254/220 nm. Purification resulted in N—((S,Z)-1-((S)-1-((2R,5′S)-5′-cyano-3-oxo-3,4-dihydrospiro[pyrido[3,2-b][1,4]oxazine-2,3′-pyrrolidin]-1′-yl)-4,4-dimethyl-1-oxopentan-2-yl)-2-oxo-1,2,3,4,7,8-hexahydroazocin-3-yl)-2,2,2-trifluoroacetamide (46.7 mg, 30%) as a white solid. ¹H NMR (400 MHz, 80° C., DMSO-d₆) δ 11.45 (br, 1H), 9.22 (br, 1H), 7.90-8.05 (m, 1H), 7.20-7.35 (m, 1H), 6.95-7.10 (m, 1H), 5.66-5.81 (m, 1H), 5.48-5.65 (m, 1H), 5.21-5.45 (m, 1H), 4.80-5.09 (m, 2H), 4.23-4.42 (m, 1H), 4.00-4.22 (m, 1H), 3.51-3.80 (m, 2H), 2.73-2.81 (m, 1H), 2.57-2.72 (m, 2H), 2.41-2.50 (m, 1H), 2.21-2.39 (m, 2H), 1.95-2.09 (m, 1H), 1.39-1.50 (m, 1H), 0.70-1.05 (m, 9H). LC-MS (ESI, m/z): 651 [M+Na]⁺.

### Example 11

### Compounds 11

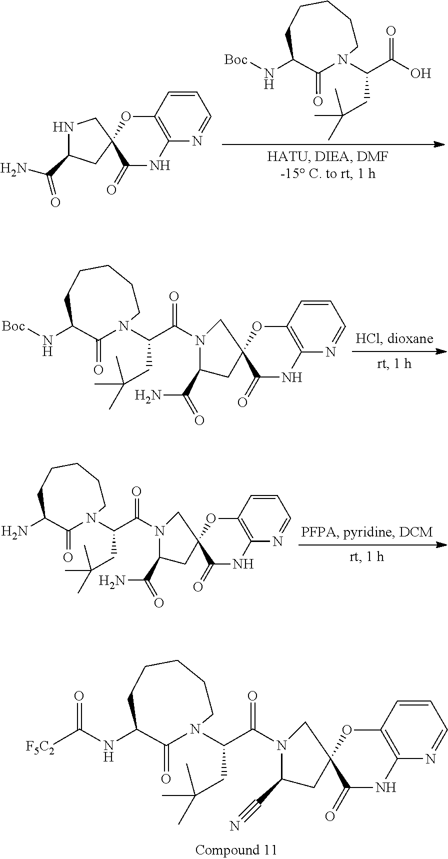

To a mixture of (S)-2-((S)-3-((tert-butoxycarbonyl)amino)-2-oxoazocan-1-yl)-4,4-dimethylpentanoic acid (160 mg, 0.432 mmol), (2R,5′S)-3-oxo-3,4-dihydrospiro[pyrido[3,2-b][1,4]oxazine-2,3′-pyrrolidine]-5′-carboxamide (107 mg, 0.432 mmol) and HATU (197 mg, 0.518 mmol) in DMF (4 mL) was added N-ethyl-N-isopropylpropan-2-amine (167 mg, 1.30 mmol) at −15° C. The mixture was stirred for 1 h from −15° C. to rt and the reaction was quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM) to provide t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)carbamate (120 mg, 46%) as an off-white solid, ¹H NMR (400 MHz, DMSO-d₆) δ 11.63 (s, 1H), 7.94-8.03 (m, 1H), 7.46 (s, 1H), 7.14-7.22 (m, 1H), 6.94-7.08 (m, 2H), 6.57-6.87 (m, 1H), 5.30-5.40 (m, 1H), 4.40-4.57 (m, 1H), 4.27-4.38 (m, 1H), 4.13-4.26 (m, 2H), 3.47-3.67 (m, 2H), 2.41-2.50 (m, 2H), 2.05-2.16 (m, 1H), 2.05-2.16 (m, 1H), 1.93-2.04 (m, 1H), 1.38-1.86 (m, 8H), 1.29-1.37 (m, 9H), 0.76-0.91 (m, 9H). LC-MS (ESI, m/z): 601 [M+H]⁺.

A mixture of tert-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)carbamate (100 mg, 0.165 mmol) in HCl (3 mL, 4.0 M in dioxane) was stirred for 1 h at rt. The mixture was concentrated under reduced pressure to afford (2R,5′S)-1′-((S)-2-((S)-3-amino-2-oxoazocan-1-yl)-4,4-dimethylpentanoyl)-3-oxo-3,4-dihydrospiro[pyrido[3,2-b][1,4]oxazine-2,3′-pyrrolidine]-5′-carboxamide (83.0 mg, crude) as an off-white solid. LC-MS (ESI, m/z): 501 [M+H]⁺.

To a mixture of (2R,5′S)-1′-((S)-2-((S)-3-amino-2-oxoazocan-1-yl)-4,4-dimethylpentanoyl)-3-oxo-3,4-dihydrospiro[pyrido[3,2-b][1,4]oxazine-2,3′-pyrrolidine]-5′-carboxamide (83.0 mg, 0.167 mmol) in DCM (3 mL) was added pyridine (79.0 mg, 1.00 mmol) and perfluoropropionic anhydride (155 mg, 0.500 mmol). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: Kinetex EVO C18, 30×150 mm, 5 m; Mobile Phase A: Water (0.1% FA), Mobile Phase B: ACN; Flow rate: 25 mL/min; Gradient (B %): 42 to 63% B in 15 min; Wave Length: 254 nm/220 nm; RT1(min): 13.28) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)-2,2,3,3,3-pentafluoropropanamide (30.5 mg, 24%) as a white solid. ¹H NMR (400 MHz, 80° C., DMSO-d₆) δ 11.45 (s, 1H), 9.25 (s, 1H), 7.95-8.05 (m, 1H), 7.20-7.27 (m, 1H), 7.00-7.10 (m, 1H), 5.30-5.40 (m, 1H), 4.80-5.05 (m, 2H), 4.20-4.35 (m, 2H), 3.50-3.75 (m, 2H), 2.73-2.82 (m, 1H), 2.56-2.72 (m, 1H), 2.05-2.25 (m, 1H), 1.70-1.85 (m, 2H), 1.58-1.69 (m, 3H), 1.48-1.57 (m, 2H), 1.38-1.47 (m, 1H), 1.28-1.37 (m, 1H), 0.75-0.95 (m, 9H). LC-MS (ESI, m/z): 651 [M+Na]⁺.

### Example 12

### Compound 12

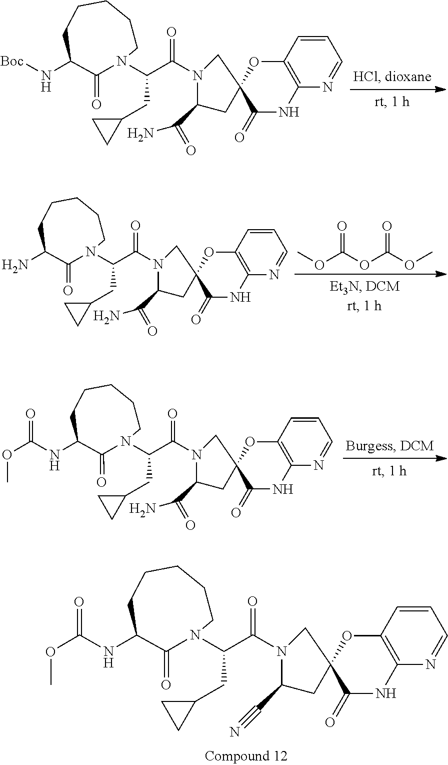

To a mixture of t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (110 mg, 0.188 mmol) in DCM (1 mL) was added HCl (5 mL, 4 M in 1,4-dioxane) was stirred for 1 h at rt and concentrated under reduced pressure to afford (2R,5′S)-1′-((S)-2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (90.0 mg, crude). LC-MS (ESI, m/z): 485 [M+H]⁺.

To a mixture of (2R,5′S)-1′-((S)-2-((S)-3-amino-2-oxoazocan-1-yl)-3-cyclopropylpropanoyl)-3-oxo-3,4-dihydrospiro[pyrido[3,2-b][1,4]oxazine-2,3′-pyrrolidine]-5′-carboxamide (90.0 mg, 0.186 mmol) in DCM (3 mL) were added triethylamine (56.0 mg, 0.558 mmol) and dimethyl dicarbonate (30.0 mg, 0.223 mmol) at 0° C. The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure to afford methyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (90.0 mg, crude) as a white solid. LC-MS (ESI, m/z): 543 [M+H]⁺.

To a mixture of methyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (90.0 mg, 0.166 mmol) in DCM (3 mL) was added Burgess reagent (237 mg, 0.996 mmol). The mixture was stirred for 1 h at rt and the reaction was quenched with water (5 mL). The mixture was extracted with DCM (3×5 mL). The organic layers were combined, washed with brine (3×5 mL), dried over anhydrous sodium sulfate, filtered and concentrated under reduced pressure to afford the crude product. The crude product was purified by prep-HPLC (Column: XSelect CSH Prep Fluoro-Phenyl, 30×150 mm, 5 m; Mobile Phase A: Water (0.1% FA), Mobile Phase B: ACN; Flow rate: 60 mL/min; Gradient: 5 to 34% B in 10 min; Wave Length: 254 nm/220 nm; Rₜ1(min): 10.2) to provide methyl ((S)-1-((S)-1-((2R,5′S)-5′-cyano-3-oxo-3,4-dihydrospiro[pyrido[3,2-b][1,4]oxazine-2,3′-pyrrolidin]-1′-yl)-3-cyclopropyl-1-oxopropan-2-yl)-2-oxoazocan-3-yl)carbamate (29.5 mg, 33%) as a white solid. ¹H NMR (400 MHz, 80° C., DMSO-d₆) δ 11.48 (br, 1H), 7.90-8.00 (m, 1H), 7.20-7.35 (m, 1H), 6.95-7.05 (m, 1H), 6.60-6.85 (m, 1H), 5.10-5.25 (m, 1H), 4.85-5.00 (m, 1H), 4.50-4.60 (m, 1H), 4.12-4.32 (m, 2H), 3.56-3.70 (m, 1H), 3.50-3.55 (m, 1H), 3.49 (s, 3H), 2.75-2.85 (m, 1H), 2.60-2.74 (m, 1H), 1.30-1.80 (m, 10H), 0.50-0.70 (m, 1H), 0.30-0.48 (m, 2H), 0.01-0.15 (m, 2H). LC-MS (ESI, m/z): 547 [M+Na]⁺.

### Example 13

### Compound 13

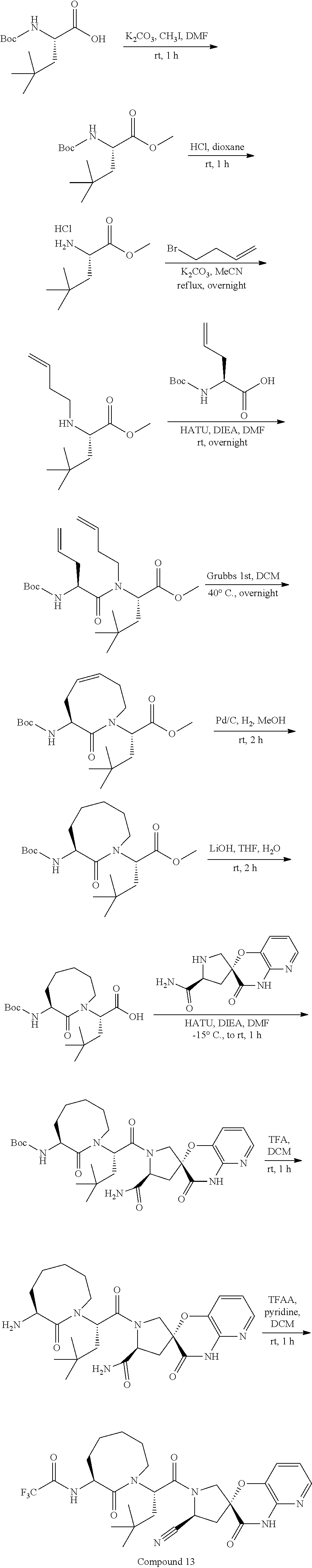

To mixture of (S)-2-((tert-butoxycarbonyl)amino)-4,4-dimethylpentanoic acid (4.70 g, 19.1 mmol) in DMF (50 mL) was added potassium carbonate (5.30 g, 38.3 mmol) and dropwise iodomethane (4.08 g, 28.7 mmol) at 0° C. The mixture was stirred for 1 h at rt. The mixture was filtered through a celite pad and washed with EtOAc. The filtrate was quenched with water (150 mL), then extracted with EtOAc (2×100 mL). The organic layers were combined, washed with brine (2×100 mL), dried over anhydrous sodium sulfate, filtered and concentrated under reduced pressure to afford methyl (S)-2-((tert-butoxycarbonyl) amino)-4,4-dimethylpentanoate (4.97 g, crude) as a yellow oil. LC-MS (ESI, z/z): 260 [M+H]⁺.

A mixture of methyl (S)-2-((t-butoxycarbonyl) amino)-4,4-dimethylpentanoate (4.97 g, 19.1 mmol) in HCl (80 mL, 4.0 M in 1,4-dioxane). The mixture was stirred for 1 h at rt and concentrated under reduced pressure to afford methyl (S)-2-amino-4,4-dimethylpentanoate hydrochloride (3.74 g, crude) as a yellow oil. LC-MS (ESI, m/z): 160 [M+H]⁺.

To a mixture of methyl (S)-2-amino-4,4-dimethylpentanoate (3.74 g, 19.1 mmol) in MeCN (35 mL) was added potassium carbonate (2.64 g, 19.1 mmol). After stirring for 30 minutes at rt, 4-bromobut-1-ene (2.57 g, 19.1 mmol) was added. The mixture was reflux overnight, then filtered through a celite pad and washed with EtOAc. The reaction was quenched with water (50 mL). The mixture was extracted with EtOAc (3×80 mL). The organic layers were combined, washed with brine (2×50 mL) and dried over anhydrous sodium sulfate, filtered and concentrated under reduced pressure to afford methyl (S)-2-(but-3-en-1-ylamino)-4,4-dimethylpentanoate (2.50 g, crude) as a light-yellow oil. LC-MS (ESI, m/z): 214 [M+H]⁺.

To a mixture of methyl (S)-2-(but-3-en-1-ylamino)-4,4-dimethylpentanoate (2.50 g, 11.7 mmol), (S)-2-((t-butoxycarbonyl)amino)pent-4-enoic acid (4.54 g, 21.0 mmol) and HATU (10.2 g, 26.9 mmol) in DMF (30 mL) was added N-ethyl-N-isopropylpropan-2-amine (7.57 g, 58.5 mmol) at 0° C. The mixture was stirred overnight at rt and quenched with water (100 mL). The mixture was extracted with EtOAc (3×100 mL). The organic layers were combined, washed with brine (2×80 mL), dried over anhydrous sodium sulfate, filtered and concentrated under reduced pressure to afford the crude product. The crude product was purified by C18 column with CH₃CN/Water (0.05% TFA). The fraction with the product was concentrated under reduced pressure to provide methyl (S)-2-((S)—N-(but-3-en-1-yl)-2-((tert-butoxycarbonyl)amino)pent-4-enamido)-4,4-dimethylpentanoate (2.80 g, 52%) as a brown oil. LC-MS (ESI, m/z): 411 [M+H]⁺.

To a mixture of methyl (S)-2-((S)—N-(but-3-en-1-yl)-2-((tert-butoxycarbonyl)amino)pent-4-enamido)-4,4-dimethylpentanoate (2.80 g, 6.82 mmol) in DCM (680 mL, 0.01 M) was added Grubbs 1st (844 mg, 1.02 mmol). The mixture was stirred overnight at 40° C. and quenched with water (500 mL). The mixture was extracted with DCM (2×500 mL). The organic layers were combined, washed with brine (2×500 mL), dried over anhydrous sodium sulfate, filtered and concentrated under reduced pressure to afford the crude product. The crude product was chromatographed on a silica gel column with EtOAc:PE (24:76) to provide methyl (S)-2-((S,Z)-3-((tert-butoxycarbonyl)amino)-2-oxo-3,4,7,8-tetrahydroazocin-1(2H)-yl)-4,4-dimethylpentanoate (1.60 g, 49%). ¹H NMR (400 MHz, DMSO-d₆) δ 6.57-7.03 (m, 1H), 5.49-5.68 (m, 1H), 5.30-5.48 (m, 1H), 4.97-5.16 (m, 1H), 4.56-4.85 (m, 1H), 3.45-3.75 (m, 5H), 2.52-2.65 (m, 2H), 2.09-2.39 (m, 2H), 1.70-1.85 (m, 1H), 1.46-1.61 (m, 1H), 1.36 (s, 9H), 0.85 (s, 9H). LC-MS (ESI, m/z): 405 [M+Na]⁺.

To a mixture of methyl (S)-2-((S,Z)-3-((tert-butoxycarbonyl)amino)-2-oxo-3,4,7,8-tetrahydroazocin-1(2H)-yl)-4,4-dimethylpentanoate (1.00 g, 2.61 mmol) in MeOH (15 mL) was added 10% palladium on activated carbon (500 mg). The mixture was stirred for 2 h at rt under hydrogen. The mixture was filtered through a celite pad and washed with MeOH. The filtrate concentrated under reduced pressure to afford methyl (S)-2-((S)-3-((tert-butoxycarbonyl)amino)-2-oxoazocan-1-yl)-4,4-dimethylpentanoate (900 mg, 85%, crude) as a yellow oil. ¹H NMR (400 MHz, DMSO-d₆) δ 6.57-6.95 (m, 1H), 5.09-5.23 (m, 1H), 4.42-4.68 (m, 1H), 3.46-3.77 (m, 4H), 3.38-3.45 (m, 1H), 1.42-2.00 (m, 8H), 1.19-1.41 (m, 11H), 0.76-0.96 (m, 9H). LC-MS (ESI, m/z): 385 [M+H]⁺.

To a mixture of methyl (S)-2-((S)-3-((t-butoxycarbonyl)amino)-2-oxoazocan-1-yl)-4,4-dimethylpentanoate (900 mg, 2.34 mmol) in THF (3 mL) was added a solution of LiOH (168 mg, 7.02 mmol) in H₂O (3 mL). The mixture was stirred for 2 h at rt and diluted with water (10 mL). The mixture was acidified to pH=6 with HCl (1 M). The mixture was extracted with EtOAc (3×10 mL). The organic layers were combined, washed with brine (10 mL), dried over anhydrous sodium sulfate, filtered and concentrated under reduced pressure to afford (S)-2-((S)-3-((tert-butoxycarbonyl)amino)-2-oxoazocan-1-yl)-4,4-dimethylpentanoic acid (800 mg, 87%, crude) as a light yellow solid. ¹H NMR (400 MHz, DMSO-d₆) δ 12.60 (br, 1H), 6.54-6.86 (m, 1H), 4.97-5.14 (m, 1H), 4.42-4.63 (m, 1H), 3.58-3.74 (m, 1H), 3.36-3.47 (m, 1H), 1.88-2.01 (m, 1H), 1.50-1.76 (m, 6H), 1.41-1.50 (m, 2H), 1.29-1.40 (m, 9H), 1.15-1.27 (m, 1H), 0.69-0.98 (m, 9H). LC-MS (ESI, m/z): 371 [M+H]⁺.

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (150 mg, 0.431 mmol) in DCM (3 mL) was added TFA (1 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (107 mg, crude) as a yellow solid. LC-MS (ESI, m/z): 271 [M+Na]⁺.

To a mixture of (S)-2-((S)-3-((tert-butoxycarbonyl)amino)-2-oxoazocan-1-yl)-4,4-dimethylpentanoic acid (160 mg, 0.432 mmol), (2R,5′S)-3-oxo-3,4-dihydrospiro[pyrido[3,2-b][1,4]oxazine-2,3′-pyrrolidine]-5′-carboxamide (107 mg, 0.432 mmol) and HATU (197 mg, 0.518 mmol) in DMF (3 mL) was added N-ethyl-N-isopropylpropan-2-amine (251 mg, 1.94 mmol) at −15° C. The mixture was stirred for 1 h from −15° C. to rt and then quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM) to afford t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)carbamate (160 mg, 58%) as a light yellow solid. ¹H NMR (400 MHz, DMSO-d₆) δ 11.64 (s, 1H), 7.94-8.02 (m, 1H), 7.46 (s, 1H), 7.14-7.24 (m, 1H), 6.96-7.10 (m, 2H), 6.57-6.88 (m, 1H), 5.32-5.41 (m, 1H), 4.42-4.55 (m, 1H), 4.26-4.37 (m, 1H), 4.13-4.26 (m, 2H), 3.47-3.64 (m, 2H), 2.41-2.49 (m, 1H), 2.04-2.16 (m, 1H), 1.93-2.03 (m, 1H), 1.40-1.76 (m, 9H), 1.31-1.39 (m, 9H), 0.81-0.92 (m, 9H). LC-MS (ESI, m/z): 623 [M+Na]⁺.

To a mixture of t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)carbamate (160 mg, 0.266 mmol) in DCM (3 mL) was added TFA (1 mL). The mixture was stirred for 1 h at rt and concentrated under reduced pressure to afford (2R,5′S)-1′-((S)-2-((S)-3-amino-2-oxoazocan-1-yl)-4,4-dimethylpentanoyl)-3-oxo-3,4-dihydrospiro[pyrido[3,2-b][1,4]oxazine-2,3′-pyrrolidine]-5′-carboxamide (133 mg, crude) as a light yellow solid. LC-MS (ESI, m/z): 523 [M+Na]⁺.

To a mixture of (2R,5′S)-1′-((S)-2-((S)-3-amino-2-oxoazocan-1-yl)-4,4-dimethylpentanoyl)-3-oxo-3,4-dihydrospiro[pyrido[3,2-b][1,4]oxazine-2,3′-pyrrolidine]-5′-carboxamide (133 mg, 0.266 mmol) in DCM (3 mL) were added pyridine (105 mg, 1.33 mmol) and trifluoroacetic anhydride (111 mg, 0.532 mmol). The mixture was stirred for 1 h at rt and quenched with water (5 mL). The mixture was extracted with DCM (3×5 mL). The organic layers were combined, washed with brine (2×5 mL), dried over anhydrous sodium sulfate, filtered and concentrated under reduced pressure to remove the solvent. The residue was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient (B %): 29 to 59% B in 10 min; Wave Length: 254 nm/220 nm; RT1(min): 9.7) to afford the crude product (~4% isomer). The crude product was separated by prep-SFC-HPLC (Column: CHIRALPAK ID, 2×25 cm, 5 m; Mobile Phase A: Hex(0.1% FA)-HPLC, Mobile Phase B: MeOH:DCM=1:1; Flow rate: 20 mL/min; Gradient (B %): isocratic 40; Wave Length: 254 nm/220 nm) to afford N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4,4-dimethyl-1-oxopentan-2-yl)-2-oxoazocan-3-yl)-2,2,2-trifluoroacetamide (46.9 mg, 30%) as a white solid. ¹H NMR (400 MHz, 80° C., DMSO-d₆) δ 11.50 (br, 1H), 9.21 (s, 1H), 7.90-8.05 (m, 1H), 7.18-7.30 (m, 1H), 6.95-7.10 (m, 1H), 5.28-5.45 (m, 1H), 4.89-5.05 (m, 1H), 4.75-4.88 (m, 1H), 4.18-4.32 (m, 2H), 3.52-3.72 (m, 2H), 2.73-2.87 (m, 1H), 2.56-2.70 (m, 1H), 2.10-2.21 (m, 1H), 1.65-1.82 (m, 2H), 1.55-1.64 (m, 3H), 1.48-1.54 (m, 2H), 1.34-1.47 (m, 1H), 1.21-1.33 (m, 1H), 0.78-0.99 (m, 9H). LC-MS (ESI, m/z): 601 [M+Na]⁺.

### Example 14

### Compounds 14

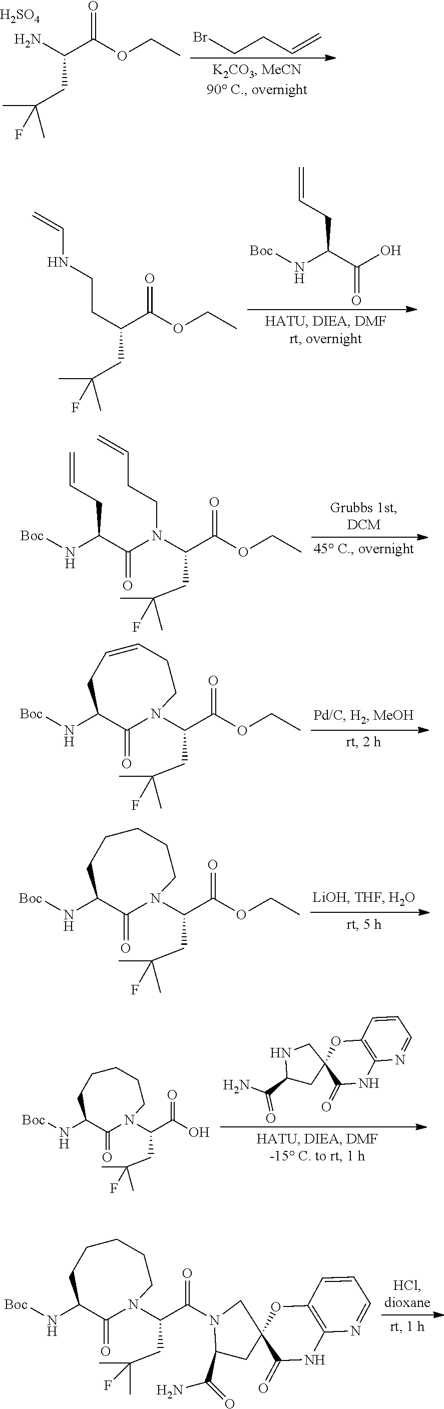

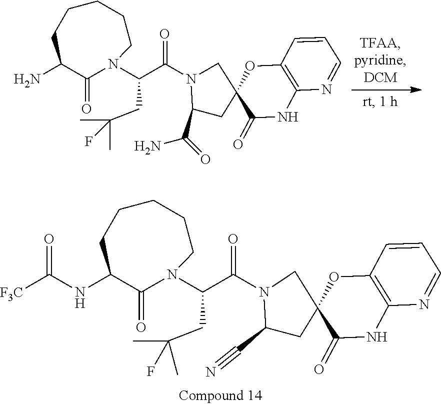 [SMILES: CC(C)(F)C[C@@H](C(=O)N1C[C@@]2(C[C@H]1C(N)=O)Oc1cccnc1NC2=O)N1CCCCC[C@H](N)C1=O ; CC(C)(F)C[C@@H](C(=O)N1C[C@@]2(C[C@H]1C#N)Oc1cccnc1NC2=O)N1CCCCC[C@H](NC(=O)C(F)(F)F)C1=O]

A mixture of ethyl (S)-2-amino-4-fluoro-4-methylpentanoate sulfate (1.8 g, 6.53 mmol) and potassium carbonate (1.99 g, 14.3 mmol) in MeCN (60 mL) was stirred for 30 min at rt. 4-bromobut-1-ene (0.970 g, 7.19 mmol) was added. The mixture was stirred overnight at 90° C. The mixture was filtered through a celite pad and quenched with water (100 mL). The mixture was extracted with EtOAc (3×100 mL). The organic layers were combined, washed with brine (100 mL), dried over anhydrous sodium sulfate, filtered and concentrated under reduced pressure to afford ethyl (S)-2-(but-3-en-1-ylamino)-4-fluoro-4-methylpentanoate (1.1 g, crude) as a yellow oil. LC-MS (ESI, m/z): 232 [M+H]⁺.

To a mixture of ethyl (S)-2-(but-3-en-1-ylamino)-4-fluoro-4-methylpentanoate (1.10 g, 4.75 mmol), HATU (4.52 g, 11.8 mmol) and (S)-2-((tert-butoxycarbonyl)amino)pent-4-enoic acid (2.05 g, 9.51 mmol) in DMF (20 mL) was added N-ethyl-N-isopropylpropan-2-amine (3.69 g, 28.5 mmol) at 0° C. The mixture was stirred overnight at rt and quenched with water (50 mL). The mixture was extracted with EtOAc (3×50 mL). The organic layers were combined, washed with brine (2×50 mL), dried over anhydrous sodium sulfate, filtered and concentrated under reduced pressure to afford the crude product. The crude product was purified by C18 column with CH₃CN/Water (0.05% TFA). The fraction with the product was concentrated under reduced pressure to provide ethyl (S)-2-((S)—N-(but-3-en-1-yl)-2-((tert-butoxycarbonyl)amino)pent-4-enamido)-4-fluoro-4-methylpentanoate (530 mg, 22%) as a yellow oil. ¹H NMR (400 MHz, DMSO-d₆) δ 6.78-7.25 (m, 1H), 5.65-5.93 (m, 2H), 5.00-5.19 (m, 4H), 4.27-4.38 (m, 2H), 3.93-4.05 (m, 2H), 3.25-3.58 (m, 2H), 2.17-2.50 (m, 6H), 1.12-1.38 (m, 18H). LC-MS (ESI, m/z): 429 [M+H]⁺.

To a mixture of ethyl (S)-2-((S)—N-(but-3-en-1-yl)-2-((tert-butoxycarbonyl)amino)pent-4-enamido)-4-fluoro-4-methylpentanoate (530 mg, 1.27 mmol) in DCM (130 mL) was added Grubbs 1st (164 mg, 0.192 mmol). The mixture was stirred overnight at 45° C. under nitrogen and quenched with water (100 mL). The mixture was extracted with DCM (2×100 mL). The organic layers were combined, washed with brine (2×200 mL), dried over anhydrous sodium sulfate, filtered and concentrated under reduced pressure. The crude product was purified by C18 column with CH₃CN/Water (0.05% TFA) to provide ethyl (S)-2-((S,Z)-3-((t-butoxycarbonyl)amino)-2-oxo-3,4,7,8-tetrahydroazocin-1(2H)-yl)-4-fluoro-4-methylpentanoate (320 mg, 56%) as a brown oil. ¹H NMR (400 MHz, DMSO-d₆) δ 6.81-6.97 (m, 1H), 5.36-5.64 (m, 2H), 4.55-4.89 (m, 2H), 3.97-4.16 (m, 2H), 3.73-3.78 (m, 1H), 3.49-3.56 (m, 1H), 2.52-2.61 (m, 2H), 2.13-2.40 (m, 3H), 1.84-1.98 (m, 1H), 1.13-1.40 (m, 18H). LC-MS (ESI, m/z): 401 [M+H]⁺.

To a mixture of ethyl (S)-2-((S,Z)-3-((tert-butoxycarbonyl)amino)-2-oxo-3,4,7,8-tetrahydroazocin-1(2H)-yl)-4-fluoro-4-methylpentanoate (320 mg, 0.799 mmol) in MeOH (6 mL) was added 10% Pd/C (320 mg). The mixture was stirred for 2 h at rt under hydrogen. The mixture was filtered through a celite pad and washed with MeOH (10 mL). The filtrate was concentrated under reduced pressure to afford the ethyl (S)-2-((S)-3-((t-butoxycarbonyl)amino)-2-oxoazocan-1-yl)-4-fluoro-4-methylpentanoate (300 mg, crude) as a light yellow oil. LC-MS (ESI, m/z): 403 [M+H]⁺.

To a mixture of ethyl (S)-2-((S)-3-((tert-butoxycarbonyl)amino)-2-oxoazocan-1-yl)-4-fluoro-4-methylpentanoate (300 mg, 0.745 mmol) in THF (6 mL) was added a solution of LiOH (54.0 mg, 2.23 mmol) in water (6 mL). The mixture was stirred for 5 h at rt and diluted with water (10 mL). The mixture was adjusted to pH=6 with HCl (1 M). The mixture was extracted with EtOAc (3×30 mL). The organic layers were combined, washed with brine (30 mL), dried over anhydrous sodium sulfate, filtered and concentrated under reduced pressure to afford (S)-2-((S)-3-((tert-butoxycarbonyl)amino)-2-oxoazocan-1-yl)-4-fluoro-4-methylpentanoic acid (230 mg, 74%, crude) as a light yellow semi-solid. LC-MS (ESI, m/z): 375 [M+H]⁺.

A mixture of t t-butyl (2R,5′S)-5′-carbamoyl-3-oxo-3,4-dihydrospiro[pyrido[3,2-b][1,4]oxazine-2,3′-pyrrolidine]-1′-carboxylate (200 mg, 0.575 mmol) in hydrogen chloride (10 mL, 4 M in 1,4-dioxane) was stirred for 1 h at rt. The mixture was concentrated under reduced pressure to afford (2R,5′S)-3-oxo-3,4-dihydrospiro[pyrido[3,2-b][1,4]oxazine-2,3′-pyrrolidine]-5′-carboxamide (142 mg, crude) as a light brown solid. LC-MS (ESI, m/z): 249 [M+H]⁺.

To a mixture of (2R,5′S)-3-oxo-3,4-dihydrospiro[pyrido[3,2-b][1,4]oxazine-2,3′-pyrrolidine]-5′-carboxamide (142 mg, 0.572 mmol), (S)-2-((S)-3-((tert-butoxycarbonyl)amino)-2-oxoazocan-1-yl)-4-fluoro-4-methylpentanoic acid (215 mg, 0.572 mmol) and HATU (261 mg, 0.686 mmol) in DMF (4 mL) was added N-ethyl-N-isopropylpropan-2-amine (296 mg, 2.28 mmol) at −15° C. The mixture was stirred for 1 h from −15° C. to rt and quenched with water (10 mL). The mixture was extracted with EtOAc (3×10 mL). The organic layers were combined, washed with brine (2×10 mL), dried over anhydrous sodium sulfate, filtered and concentrated under reduced pressure. The crude product was purified by TLC (Mobile phase: MeOH:DCM) to afford t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-fluoro-4-methyl-1-oxopentan-2-yl)-2-oxoazocan-3-yl)carbamate (150 mg, 39%) as a light yellow solid. ¹H NMR (400 MHz, DMSO-d₆) δ 11.64 (s, 1H), 7.94-8.03 (m, 1H), 7.45-7.52 (m, 1H), 7.17-7.25 (m, 1H), 7.00-7.10 (m, 2H), 6.84-6.91 (m, 1H), 5.41-5.53 (m, 1H), 4.14-4.60 (m, 4H), 3.47-3.66 (m, 2H), 2.43-2.49 (m, 1H), 2.06-2.37 (m, 2H), 1.24-1.71 (m, 24H). LC-MS (ESI, m/z): 627 [M+Na]⁺.

A mixture of t-butyl ((S)-1-((S)-1-((2R,5′S)-5′-carbamoyl-3-oxo-3,4-dihydrospiro[pyrido[3,2-b][1,4]oxazine-2,3′-pyrrolidin]-1′-yl)-4-fluoro-4-methyl-1-oxopentan-2-yl)-2-oxoazocan-3-yl)carbamate (150 mg, 0.248 mmol) in HCl (10 mL, 4 M in 1,4-dioxane) was stirred for 1 h at rt. The mixture was concentrated under reduced pressure to afford N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-fluoro-4-methyl-1-oxopentan-2-yl)-2-oxoazocan-3-yl)-2,2,2-trifluoroacetamide (125 mg, crude) as a yellow solid. LC-MS (ESI, m/z): 505 [M+H]⁺.

To a solution of N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-fluoro-4-methyl-1-oxopentan-2-yl)-2-oxoazocan-3-yl)-2,2,2-trifluoroacetamide (125 mg, 0.248 mmol) in DCM (3 mL) were added pyridine (98.0 mg, 1.24 mmol) and trifluoroacetic anhydride (105 mg, 0.496 mmol). The mixture was stirred for 1 h at rt and quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product. The crude product was purified by prep-HPLC (Column: Kinetex EVO C18, 30×150 mm, 5 m; Mobile Phase A: Water (0.1% FA), Mobile Phase B: ACN; Flow rate: 25 mL/min; Gradient (B %): 31 to 52% B in 10 min; Wave Length: 254 nm/220 nm; RT1(min): 9.72) to provide the product (30 mg, 95% pure, mixture of ~5% isomer). The product was separated by Chiral-HPLC (Column: CHIRALPAK ID, 2×25 cm, m; Mobile Phase A: Hex-HPLC, Mobile Phase B: MeOH:DCM=1:1; Flow rate: 20 mL/min; Gradient (B %): isocratic 40; Wave Length: 254 nm/220 nm) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-fluoro-4-methyl-1-oxopentan-2-yl)-2-oxoazocan-3-yl)-2,2,2-trifluoroacetamide (23.4 mg, 16%) as a white solid. ¹H NMR (400 MHz, 100° C., DMSO-d₆) δ 11.35 (br, 1H), 9.00-9.10 (m, 1H), 7.90-8.10 (m, 1H), 7.15-7.30 (m, 1H), 6.95-7.10 (m, 1H), 5.40-5.50 (m, 1H), 4.90-5.10 (m, 1H), 4.80-4.89 (m, 1H), 4.20-4.30 (m, 2H), 3.65-3.75 (m, 1H), 3.55-3.64 (m, 1H), 2.75-2.85 (m, 1H), 2.60-2.70 (m, 1H), 2.40-2.50 (m, 1H), 1.70-1.90 (m, 3H), 1.45-1.69 (m, 6H), 1.30-1.35 (m, 3H), 1.20-1.29 (m, 3H). LC-MS (ESI, m/z): 605 [M+Na]⁺.

### Example 15

### Compound 15

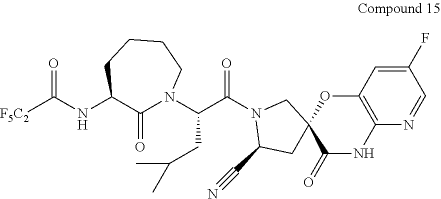

A mixture of tert-butyl ((S)-1-((S)-1-((2R,5′S)-5′-carbamoyl-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)carbamate (110 mg, 0.182 mmol) in HCl (3 mL, 4 M in 1,4-dioxane) was stirred for 1 h at rt and concentrated under reduced pressure to afford (2R,5′S)-1′-((S)-2-((S)-3-amino-2-oxoazocan-1-yl)-4-methylpentanoyl)-7-fluoro-3-oxo-3,4-dihydrospiro[pyrido[3,2-b][1,4]oxazine-2,3′-pyrrolidine]-5′-carboxamide (crude, 92.0 mg) as an off-white solid. LC-MS (ESI, m/z): 505 [M+H]⁺.

To a mixture of (2R,5′S)-1′-((S)-2-((S)-3-amino-2-oxoazocan-1-yl)-4-methylpentanoyl)-7-fluoro-3-oxo-3,4-dihydrospiro[pyrido[3,2-b][1,4]oxazine-2,3′-pyrrolidine]-5′-carboxamide (92.0 mg, 0.182 mmol, 1.0 eq.) in DCM (2 mL, 0.182 mmol, 1.0 eq.) was added pyridine (72.0 mg, 0.910 mmol, 5.0 eq.) and perfluoropropionic anhydride (113 mg, 0.364 mmol, 2.0 eq.). The mixture was stirred for 1 h at rt and the reaction was quenched with water (5 mL). The mixture was extracted with DCM (3×5 mL). The organic layers were combined, washed with brine (2×5 mL), dried over anhydrous sodium sulfate, filtered and concentrated under reduced pressure. The crude product was purified by prep-HPLC (Column: CHIRAL ART Cellulose-SZ 3.0×50 mm, 3 m; Mobile Phase B (isocratic): CH₃OH (1% 2M NH₃-MeOH); Flow rate: 2 mL/min; to provide N—((S)-1-((S)-1-((2R,5′S)-5′-cyano-7-fluoro-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2,3,3,3-pentafluoropropanamide (10.5 mg, 9%) as a white solid. ¹H NMR (400 MHz, 80° C., DMSO-d₆) δ 9.41 (s, 1H), 8.03 (s, 1H), 7.20-7.40 (m, 1H), 5.20-5.40 (m, 1H), 4.70-5.00 (m, 2H), 4.01-4.40 (m, 2H), 3.60-3.80 (m, 1H), 3.48-3.59 (m, 1H), 2.80-2.90 (m, 1H), 2.65-2.71 (m, 1H), 1.31-1.85 (m, 11H), 0.82-1.00 (m, 3H), 0.68-0.81 (m, 3H). LC-MS (ESI, m/z): 655 [M+Na]⁺.

### Example 16

### Compounds 16

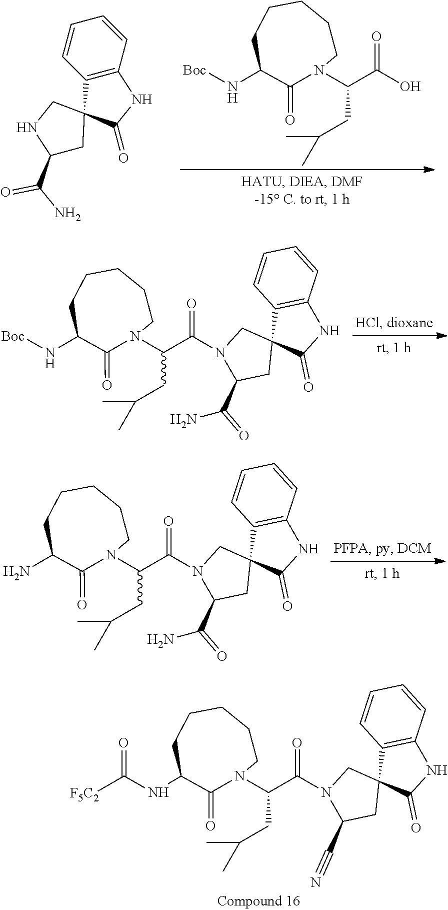

To a mixture of (3R,5′S)-2-oxospiro[indoline-3,3′-pyrrolidine]-5′-carboxamide (105 mg, 0.454 mmol), (S)-2-((S)-3-((tert-butoxycarbonyl)amino)-2-oxoazocan-1-yl)-4-methylpentanoic acid (162 mg, 0.454 mmol) and HATU (207 mg, 0.545 mmol) in DMF (3 mL) was added N-ethyl-N-isopropylpropan-2-amine (176 mg, 1.36 mmol) at −15° C. The mixture was stirred for 1 h from −15° C. to rt and quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:11; Rf=0.4; detection: UV) to provide t-butyl ((3S)-1-(1-((3R,5′S)-5′-carbamoyl-2-oxospiro[indoline-3,3′-pyrrolidin]-1′-yl)-4-methyl-1-oxopentan-2-yl)-2-oxoazocan-3-yl)carbamate (190 mg, 73%) as an off-white solid. ¹H NMR (400 MHz, DMSO-d₆) δ 10.49-10.79 (m, 1H), 7.42-7.58 (m, 1H), 7.18-7.28 (m, 1H), 7.02-7.12 (m, 1H), 6.70-7.02 (m, 4H), 5.07-5.37 (m, 1H), 4.55-4.72 (m, 1H), 4.37-4.54 (m, 1H), 3.63-3.91 (m, 2H), 3.35-3.61 (m, 2H), 2.03-2.27 (m, 2H), 1.40-1.76 (m, 9H), 1.26-1.37 (m, 10H), 0.62-0.95 (m, 7H). LC-MS (ESI, m/z): 570 [M+H]⁺.

A mixture of tert-butyl ((3S)-1-(1-((3R,5′S)-5′-carbamoyl-2-oxospiro[indoline-3,3′-pyrrolidin]-1′-yl)-4-methyl-1-oxopentan-2-yl)-2-oxoazocan-3-yl)carbamate (190 mg, 0.334 mmol) in HCl (5 mL, 4 M in dioxane) was stirred for 1 h at rt. The mixture was concentrated under reduced pressure to afford (3R,5′S)-1′-(2-((S)-3-amino-2-oxoazocan-1-yl)-4-methylpentanoyl)-2-oxospiro[indoline-3,3′-pyrrolidine]-5′-carboxamide (157 mg, crude) as an off-white solid. LC-MS (ESI, m/z): 470 [M+H]⁺.

To a mixture of (3R,5′S)-1′-(2-((S)-3-amino-2-oxoazocan-1-yl)-4-methylpentanoyl)-2-oxospiro[indoline-3,3′-pyrrolidine]-5′-carboxamide (156 mg, 0.334 mmol) in DCM (5 mL) were added pyridine (132 mg, 1.67 mmol) and pentafluoropropionic anhydride (259 mg, 0.835 mmol). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was first separated by prep-CHIRAL-SFC (Column: GreenSep Basic 3×15 cm, 5 m; Mobile Phase A: CO₂, Mobile Phase B: ACN:MeOH=4:1 (1% 2M NH₃-MeOH); Flow rate: 70 mL/min; Gradient (B %): isocratic 24% B; Column Temperature (° C.): 35; Wave Length: 220 nm; RT1(min): 4.3; RT2(min): 5.57). The product was separated by CHIRAL-HPLC (Column: CHIRAL ART Cellulose-SB, 2×25 cm, 5 m; Mobile Phase A: Hex, Mobile Phase B: EtOH:DCM=1:1; Flow rate: 20 mL/min; Gradient (B %): isocratic 15; Wave Length: 254 nm/220 nm) to provide N—((S)-1-((S)-1-((3R,5′S)-5′-cyano-2-oxospiro[indoline-3,3′-pyrrolidin]-1′-yl)-4-methyl-1-oxopentan-2-yl)-2-oxoazocan-3-yl)-2,2,3,3,3-pentafluoropropanamide (40.5 mg, 20%) as a white solid. ¹H NMR (400 MHz, 80° C., DMSO-d₆) δ 10.49 (s, 1H), 9.25 (s, 1H), 7.20-7.30 (m, 1H), 7.00-7.10 (m, 1H), 6.80-6.95 (m, 2H), 5.20-5.35 (m, 1H), 5.05-5.18 (m, 1H), 4.75-4.90 (m, 1H), 3.85-4.00 (m, 2H), 3.50-3.70 (m, 2H), 2.58-2.70 (m, 1H), 2.48-2.57 (m, 1H), 1.40-1.70 (m, 10H), 1.05-1.20 (m, 1H), 0.82-0.95 (m, 3H), 0.70-0.81 (m, 3H). LC-MS (ESI, m/z): 620 [M+Na]⁺.

### Example 17

### Compound 17

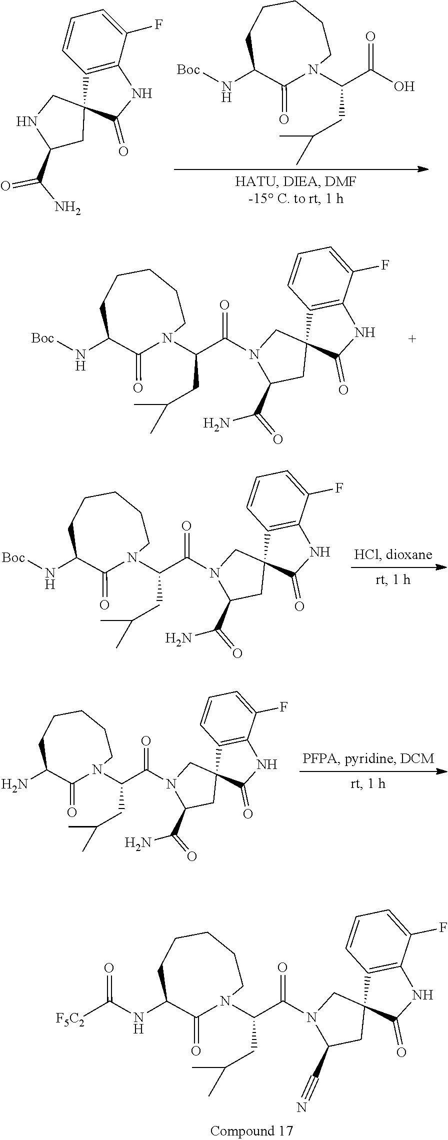

To a mixture of (3R,5′S)-7-fluoro-2-oxospiro[indoline-3,3′-pyrrolidine]-5′-carboxamide (107 mg, 0.429 mmol), (S)-2-((S)-3-((tert-butoxycarbonyl)amino)-2-oxoazocan-1-yl)-4-methylpentanoic acid (153 mg, 0.429 mmol), HATU (196 mg, 0.515 mmol) in DMF (3 mL) was added N-ethyl-N-isopropylpropan-2-amine (167 mg, 1.29 mmol) at −15° C. The mixture was stirred for 1 h at rt and quenched with water (10 mL). The mixture was extracted with EtOAc (3×10 mL). The organic layers were combined, washed with brine (2×10 mL), dried over anhydrous sodium sulfate, filtered and concentrated under reduced. The crude product was purified by TLC (Mobile phase: MeOH:DCM=1:10; Rf1=0.45; Rf2=0.40; detection: UV) to provide tert-butyl ((S)-1-((S)-1-((3R,5′S)-5′-carbamoyl-7-fluoro-2-oxospiro[indoline-3,3′-pyrrolidin]-1′-yl)-4-methyl-1-oxopentan-2-yl)-2-oxoazocan-3-yl)carbamate (70.0 mg, 27%) as an off-white solid. LC-MS (ESI, m/z): 588 [M+H]⁺.

A mixture of tert-butyl ((S)-1-((S)-1-((3R,5′S)-5′-carbamoyl-7-fluoro-2-oxospiro[indoline-3,3′-pyrrolidin]-1′-yl)-4-methyl-1-oxopentan-2-yl)-2-oxoazocan-3-yl)carbamate (70.0 mg, 0.119 mmol) in hydrogen chloride (5 mL, 4 M in dioxane) was stirred for 1 h at rt and concentrated under reduced pressure to afford (3R,5′S)-1′-((S)-2-((S)-3-amino-2-oxoazocan-1-yl)-4-methylpentanoyl)-7-fluoro-2-oxospiro[indoline-3,3′-pyrrolidine]-5′-carboxamide (58.0 mg, crude) as an off-white solid. LC-MS (ESI, m/z): 488 [M+H]⁺.

To a mixture of (3R,5′S)-1′-((S)-2-((S)-3-amino-2-oxoazocan-1-yl)-4-methylpentanoyl)-7-fluoro-2-oxospiro[indoline-3,3′-pyrrolidine]-5′-carboxamide (58.0 mg, 0.119 mmol) in DCM (3 mL) was added pyridine (47.0 mg, 0.595 mmol) and perfluoropropionic anhydride (74.0 mg, 0.238 mmol). The mixture was stirred for 1 h at rt and quenched with water (5 mL). The mixture was extracted with DCM (3×5 mL). The organic layers were combined, washed with brine (2×5 mL), dried over anhydrous sodium sulfate, filtered and concentrated under reduced pressure to afford the crude product. The crude product was purified by TLC (Mobile phase: MeOH/DCM=1:10; Rf=0.4; detection: UV) to provide the product (95% pure). The product was further purified by prep-Achiral-SFC-HPLC (Column: DAICEL DCpak P4VP 3×25 cm, 5 m; Mobile Phase A: CO₂, Mobile Phase B: MeOH(0.3%-7M-NH3-MeOH); Flow rate: 65 mL/min; Gradient (B %): isocratic 26% B; Column Temperature(° C.): 35; Wave Length: 220 nm) to provide N—((S)-1-((S)-1-((3R,5′S)-5′-cyano-7-fluoro-2-oxospiro[indoline-3,3′-pyrrolidin]-1′-yl)-4-methyl-1-oxopentan-2-yl)-2-oxoazocan-3-yl)-2,2,3,3,3-pentafluoropropanamide (9.2 mg, 12%) as a white solid. ¹H NMR (400 MHz, 80° C., DMSO-d₆) δ 11.02 (br, 1H), 9.24 (br, 1H), 7.12-7.22 (m, 1H), 6.88-7.02 (m, 2H), 5.22-5.35 (m, 1H), 5.05-5.21 (m, 1H), 4.75-4.91 (m, 1H), 3.90-4.10 (m, 2H), 3.60-3.80 (m, 1H), 3.48-3.59 (m, 1H), 2.60-2.71 (m, 1H), 2.50-2.59 (m, 1H), 1.40-1.85 (m, 10H), 1.10-1.25 (m, 1H), 0.85-0.95 (m, 3H), 0.70-0.84 (m, 3H). LC-MS (ESI, m/z): 638 [M+Na]⁺.

### Example 18

### Compound 18

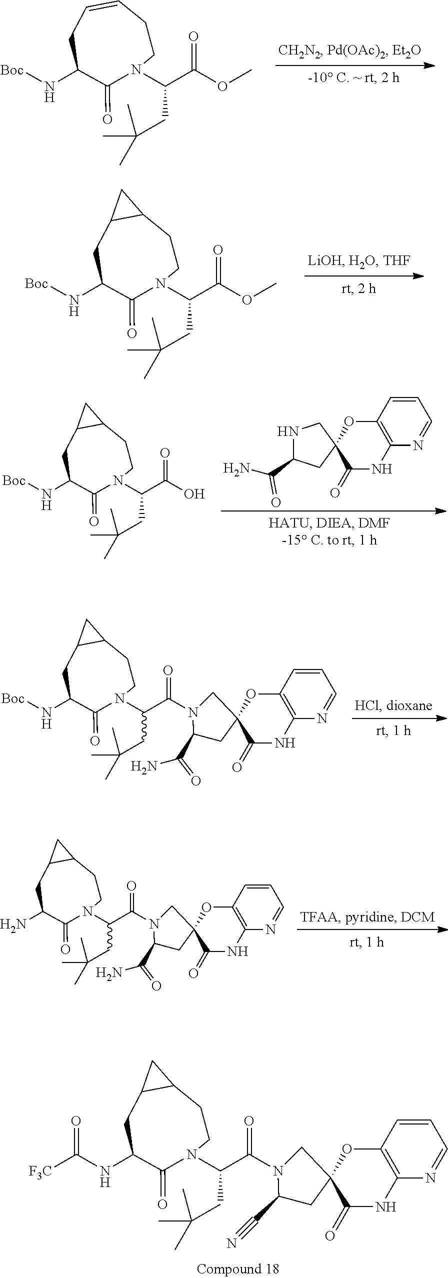

To a mixture of methyl (S)-2-((S,Z)-3-((tert-butoxycarbonyl)amino)-2-oxo-3,4,7,8-tetrahydroazocin-1(2H)-yl)-4,4-dimethylpentanoate (280 mg, 0.732 mmol) and palladium (II) acetate (82.0 mg, 0.366 mmol) in Et₂O (4 mL) was added diazomethane (4 mL, ~1M in Et₂O, excess) at −10° C. under nitrogen. The mixture was stirred for 2 h at rt. The mixture was filtered through a celite pad and washed with Et₂O. The combined organic layers were washed with H₂O (10 mL), brine (10 mL), dried over anhydrous sodium sulfate, filtered and concentrated under reduced pressure to afford methyl (S)-2-((1RS,6S,8SR)-6-((tert-butoxycarbonyl)amino)-5-oxo-4-azabicyclo[6.1.0]nonan-4-yl)-4,4-dimethylpentanoate (290 mg, crude) as a light yellow semi-solid. ¹H NMR (400 MHz, DMSO-d₆) δ 6.68-6.86 (m, 1H), 4.99-5.29 (m, 1H), 4.37-4.48 (m, 1H), 3.57-3.62 (m, 3H), 3.36-3.56 (m, 2H), 2.20-2.35 (m, 1H), 2.02-2.14 (m, 1H), 1.78-1.93 (m, 2H), 1.46-1.73 (m, 2H), 1.34-1.39 (m, 9H), 1.22-1.26 (m, 1H), 0.99-1.07 (m, 1H), 0.84-0.89 (m, 9H), 0.58-0.73 (m, 2H). LC-MS (ESI, m/z): 397 [M+H]⁺.

To a mixture of methyl (S)-2-((1RS,6S,8SR)-6-((tert-butoxycarbonyl)amino)-5-oxo-4-azabicyclo[6.1.0]nonan-4-yl)-4,4-dimethylpentanoate (290 mg, 0.731 mmol) in THF (5 mL) was added a solution of LiOH (87.0 mg, 3.65 mmol) in H₂O (5 mL). The mixture was stirred for 2 h at rt and diluted with water (10 mL). The mixture was acidified to pH=3 with HCl (1 M). The mixture was extracted with EtOAc (3×20 mL). The organic layers were combined, dried over anhydrous sodium sulfate, filtered and concentrated under reduced pressure to afford (S)-2-((1RS,6S,8SR)-6-((tert-butoxycarbonyl)amino)-5-oxo-4-azabicyclo[6.1.0]nonan-4-yl)-4,4-dimethylpentanoic acid (220 mg, crude) as a light yellow oil. LC-MS (ESI, m/z): 405 [M+Na]⁺.

A mixture of tert-butyl (2R,5′S)-5′-carbamoyl-3-oxo-3,4-dihydrospiro[pyrido[3,2-b][1,4]oxazine-2,3′-pyrrolidine]-1′-carboxylate (250 mg, 0.718 mmol) in hydrogen chloride (8 mL, 4 M in dioxane) was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (178 mg, crude) as a light yellow solid. LC-MS (ESI, m/z): 271 [M+Na]⁺.

To a mixture of (2R,5′S)-3-oxo-3,4-dihydrospiro[pyrido[3,2-b][1,4]oxazine-2,3′-pyrrolidine]-5′-carboxamide (181 mg, 0.732 mmol), (S)-2-((1RS,6S,8SR)-6-((tert-butoxycarbonyl)amino)-5-oxo-4-azabicyclo[6.1.0]nonan-4-yl)-4,4-dimethylpentanoic acid (280 mg, 0.732 mmol) and HATU (334 mg, 0.878 mmol) in DMF (4 mL) was added N-ethyl-N-isopropylpropan-2-amine (567 mg, 4.39 mmol) at −15° C. The mixture was stirred for 1 h at rt and quenched with water (20 mL). The mixture was extracted with EtOAc (3×20 mL). The organic layers were combined, washed with brine (2×20 mL), dried over anhydrous sodium sulfate, filtered and concentrated under reduced pressure. The crude product was chromatographed on a silica gel column with MeOH:DCM (10:90) to provide t-butyl ((1RS,6S,8SR)-4-(1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-5-oxo-4-azabicyclo[6.1.0]nonan-6-yl)carbamate (220 mg, 49%) as a light yellow solid. LC-MS (ESI, m/z): 635 [M+Na]⁺.

A mixture of tert-butyl ((1RS,6S,8SR)-4-(1-((2R,5′S)-5′-carbamoyl-3-oxo-3,4-dihydrospiro[pyrido[3,2-b][1,4]oxazine-2,3′-pyrrolidin]-1′-yl)-4,4-dimethyl-1-oxopentan-2-yl)-5-oxo-4-azabicyclo[6.1.0]nonan-6-yl)carbamate (220 mg, 0.359 mmol) in HCl (6 mL, 4 M in dioxane) was stirred for 1 h at rt and concentrated under reduced pressure to afford (2R,5′S)-1′-(2-((1RS,6S,8SR)-6-amino-5-oxo-4-azabicyclo[6.1.0]nonan-4-yl)-4,4-dimethylpentanoyl)-3-oxo-3,4-dihydrospiro[pyrido[3,2-b][1,4]oxazine-2,3′-pyrrolidine]-5′-carboxamide (180 mg, crude) as an off-white solid. LC-MS (ESI, m/z): 535 [M+Na]⁺.

To a mixture of (2R,5′S)-1′-(2-((1RS,6S,8SR)-6-amino-5-oxo-4-azabicyclo[6.1.0]nonan-4-yl)-4,4-dimethylpentanoyl)-3-oxo-3,4-dihydrospiro[pyrido[3,2-b][1,4]oxazine-2,3′-pyrrolidine]-5′-carboxamide (180 mg, 0.351 mmol) in DCM (5 mL) were added pyridine (139 mg, 1.75 mmol) and trifluoroacetic anhydride (147 mg, 0.702 mmol). The mixture was stirred for 1 h at rt and quenched with water (10 mL). The mixture was extracted with DCM (3×10 mL). The organic layers were combined, washed with brine (10 mL), dried over anhydrous sodium sulfate, filtered and concentrated under reduced pressure. The crude product was purified by TLC (Mobile phase: EtOAc:PE=1:1). Then was further purified by prep-HPLC (Column: Kinetex EVO C18,30×150 mm, 5 m; Mobile Phase A: Water (0.1% FA), Mobile Phase B: ACN; Flow rate: 25 mL/min; Gradient (B %): 45 to 55% B in 12 min; Wave Length: 254 nm/220 nm; to afford N-((1RS,6S,8SR)-4-(1-((2R,5′S)-5′-cyano-3-oxo-3,4-dihydrospiro[pyrido[3,2-b][1,4]oxazine-2,3′-pyrrolidin]-1′-yl)-4,4-dimethyl-1-oxopentan-2-yl)-5-oxo-4-azabicyclo[6.1.0]nonan-6-yl)-2,2,2-trifluoroacetamide (5.2 mg, 2%) as a white solid. ¹H NMR (400 MHz, 80° C., DMSO-d₆) δ 11.49 (br, 1H), 9.00-9.24 (m, 1H), 7.92-8.03 (m, 1H), 7.17-7.32 (m, 1H), 6.90-7.10 (m, 1H), 5.15-5.38 (m, 1H), 4.70-5.10 (m, 2H), 4.20-4.40 (m, 1H), 3.90-4.05 (m, 1H), 3.50-3.78 (m, 2H), 2.64-2.88 (m, 2H), 2.24-2.42 (m, 2H), 2.18-2.22 (m, 1H), 2.01-2.12 (m, 1H), 1.25-1.45 (m, 2H), 1.08-1.24 (m, 2H), 0.80-0.94 (m, 10H), 0.70-0.79 (m, 1H). LC-MS (ESI, m/z): 613 [M+Na]⁺.

### Example 19

### Compound 19

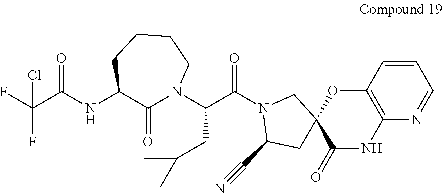 [SMILES: CC(C)C[C@@H](C(=O)N1C[C@@]2(C[C@H]1C#N)Oc1cccnc1NC2=O)N1CCCC[C@H](NC(=O)C(F)(F)Cl)C1=O]

To a mixture of (2R,5′S)-1′-((S)-2-((S)-3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (107 mg, 0.220 mmol) in DCM (3 mL) was added pyridine (87.0 mg, 1.10 mmol) and 2-chloro-2,2-difluoroacetic anhydride (107 mg, 0.440 mmol). The mixture was stirred for 1 h at rt and quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by Prep-Achiral SFC (Column: Viridis BEH Prep 2-EP OBD, 3×15 cm, 5 μm; Mobile Phase A: CO₂, Mobile Phase B: IPA(0.3%-7M-NH3-MeOH); Flow rate: 65 mL/min; Gradient (B %): isocratic 18% B; Column Temperature(° C.): 35; Wave Length: 220 nm) to provide 2-chloro-N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2,2-difluoroacetamide (15.1 mg, 11%) as a white solid. ¹H NMR (400 MHz, 100° C., DMSO-d₆) δ 11.32 (br, 1H), 8.82 (m, 1H), 7.90-8.00 (m, 1H), 7.20-7.30 (m, 1H), 6.95-7.05 (m, 1H), 5.25-5.35 (m, 1H), 4.89-5.00 (m, 1H), 4.80-4.88 (m, 1H), 4.20-4.30 (m, 2H), 3.60-3.70 (m, 1H), 3.50-3.59 (m, 1H), 2.75-2.85 (m, 1H), 2.60-2.74 (m, 1H), 1.40-1.85 (m, 11H), 0.90-1.00 (m, 3H), 0.80-0.89 (m, 3H). LC-MS (ESI, m/z): 603 [M+Na]⁺.

### Example 20

### Compound 20

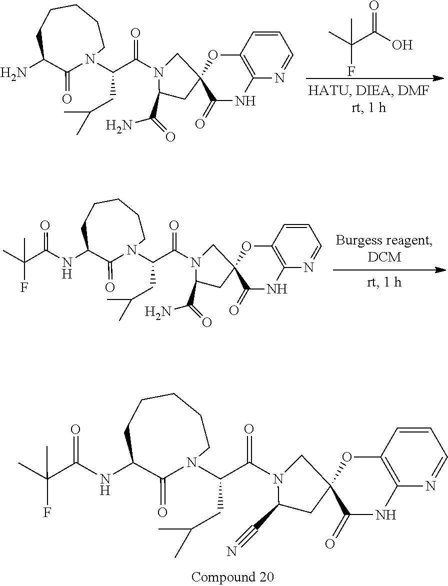

To a mixture of (2R,5′S)-1′-((S)-2-((S)-3-amino-2-oxoazocan-1-yl)-4-methylpentanoyl)-3-oxo-3,4-dihydrospiro[pyrido[3,2-b][1,4]oxazine-2,3′-pyrrolidine]-5′-carboxamide (107 mg, 0.220 mmol), 2-fluoro-2-methylpropanoic acid (24.0 mg, 0.220 mmol) and HATU (101 mg, 0.264 mmol) in DMF (3 mL) was added N-ethyl-N-isopropylpropan-2-amine (127 mg, 0.990 mmol) at 0° C. The mixture was stirred for 1 h at rt and quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:12; Rf=0.3; detection: UV) to provide (2R,5′S)-1′-((S)-2-((S)-3-(2-fluoro-2-methylpropanamido)-2-oxoazocan-1-yl)-4-methylpentanoyl)-3-oxo-3,4-dihydrospiro[pyrido[3,2-b][1,4]oxazine-2,3′-pyrrolidine]-5′-carboxamide (40.0 mg, 26%) as a light yellow solid. ¹H NMR (400 MHz, DMSO-d₆) δ 11.62 (s, 1H), 7.92-8.05 (m, 1H), 7.74-7.88 (m, 1H), 7.45-7.52 (m, 1H), 7.16-7.28 (m, 1H), 6.95-7.08 (m, 2H), 5.23-5.34 (m, 1H), 4.74-4.81 (m, 1H), 4.28-4.37 (m, 1H), 4.14-4.25 (m, 2H), 3.46-3.65 (m, 2H), 2.44-2.48 (m, 1H), 2.10-2.20 (m, 1H), 1.36-1.58 (m, 17H), 0.78-0.88 (m, 6H). LC-MS (ESI, m/z): 597 [M+Na]⁺.

To a mixture of (2R,5′S)-1′-((S)-2-((S)-3-(2-fluoro-2-methylpropanamido)-2-oxoazocan-1-yl)-4-methylpentanoyl)-3-oxo-3,4-dihydrospiro[pyrido[3,2-b][1,4]oxazine-2,3′-pyrrolidine]-5′-carboxamide (40.0 mg, 0.070 mmol) in DCM (2 mL) was added Burgess reagent (50.0 mg, 0.210 mmol). The mixture was stirred for 1 h at rt and quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by prep-HPLC (Column: XBridge Prep Shield RP18 OBD C18 Column, 30×150 mm, 5 m; Mobile Phase A: Water (0.1% FA), Mobile Phase B: ACN; Flow rate: 60 mL/min mL/min; Gradient (B %): 24 to 54% B in 10 min; Wave Length: 254 nm/220 nm; RT1(min): 9.25) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazocan-3-yl)-2-fluoro-2-methylpropanamide (11.3 mg, 28%) as a white solid. ¹H NMR (400 MHz, 100° C., DMSO-d₆) δ 11.35 (br, 1H), 7.90-8.00 (m, 1H), 7.30-7.40 (m, 1H), 7.15-7.25 (m, 1H), 6.95-7.05 (m, 1H), 5.20-5.30 (m, 1H), 4.91-5.00 (m, 1H), 4.80-4.90 (m, 1H), 4.15-4.30 (m, 2H), 3.59-3.70 (m, 1H), 3.45-3.58 (m, 1H), 2.75-2.85 (m, 1H), 2.65-2.74 (m, 1H), 1.70-1.88 (m, 1H), 1.35-1.69 (m, 16H), 0.80-0.95 (m, 6H). LC-MS (ESI, m/z): 579 [M+Na]⁺.

### Example 21

### Compound 21

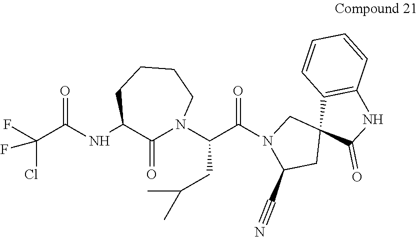 [SMILES: CC(C)C[C@@H](C(=O)N1C[C@]2(C[C@H]1C#N)C(=O)Nc1ccccc12)N1CCCC[C@H](NC(=O)C(F)(F)Cl)C1=O]

To a mixture of (3R,5′S)-1′-(2-((S)-3-amino-2-oxoazocan-1-yl)-4-methylpentanoyl)-2-oxospiro[indoline-3,3′-pyrrolidine]-5′-carboxamide (98.0 mg, 0.209 mmol) in DCM (3 mL) were added pyridine (82.0 mg, 1.04 mmol) and 2-chloro-2,2-difluoroacetic anhydride (101 mg, 0.418 mmol). The mixture was stirred for 1 h at rt and quenched with water (10 mL). The mixture was extracted with DCM (3×10 mL). The organic layers were combined, washed with brine (2×10 mL), dried over anhydrous sodium sulfate, filtered and concentrated under reduced pressure. The crude product was purified by prep-AChiral HPLC (Column: DAICEL DCpak P4VP 3×25 cm, 5 m; Mobile Phase A: CO₂, Mobile Phase B: IPA(0.3%-7M-NH₃-MeOH); Flow rate: 65 mL/min; Gradient (B %): isocratic 32% B; Column Temperature(° C.): 35; Wave Length: 220 nm; RT1(min): 5.82) and then separated by prep-Chiral HPLC (Column: Chiral ART Cellulose-SA, 2×25 cm, 5 m; Mobile Phase A: Hex-HPLC, Mobile Phase B: MeOH:DCM=1:1; Flow rate: 20 mL/min; Gradient (B %): isocratic 20; Wave Length: 254/220 nm). to afford 2-chloro-N—((S)-1-((S)-1-((3R,5′S)-5′-cyano-2-oxospiro[indoline-3,3′-pyrrolidin]-1′-yl)-4-methyl-1-oxopentan-2-yl)-2-oxoazocan-3-yl)-2,2-difluoroacetamide (12.0 mg, 10%) as a white solid. ¹H NMR (400 MHz, 80° C., DMSO-d₆) δ 10.53 (br, 1H), 8.90-9.10 (m, 1H), 7.20-7.30 (m, 1H), 7.01-7.10 (m, 1H), 6.90-7.00 (m, 1H), 6.80-6.89 (m, 1H), 5.20-5.37 (m, 1H), 5.00-5.19 (m, 1H), 4.70-4.84 (m, 1H), 3.88-4.00 (m, 2H), 3.50-3.70 (m, 2H), 2.60-2.70 (m, 1H), 2.50-2.58 (m, 1H), 1.40-1.80 (m, 10H), 1.09-1.27 (m, 1H), 0.88-0.95 (m, 3H), 0.77-0.87 (m, 3H). LC-MS (ESI, m/z): 586 [M+Na]⁺.

### Example 22

### Compound 22

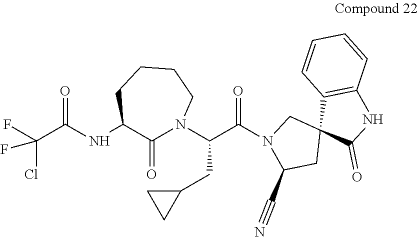 [SMILES: N#C[C@@H]1C[C@@]2(CN1C(=O)[C@H](CC1CC1)N1CCCC[C@H](NC(=O)C(F)(F)Cl)C1=O)C(=O)Nc1ccccc12]

To a mixture of (3R,5′S)-1′-((S)-2-((S)-3-amino-2-oxoazocan-1-yl)-3-cyclopropylpropanoyl)-2-oxospiro[indoline-3,3′-pyrrolidine]-5′-carboxamide (132 mg, 0.282 mmol) in DCM (4 mL) were added pyridine (112 mg, 1.41 mmol) and 2-chloro-2,2-difluoroacetic anhydride (137 mg, 0.564 mmol). The mixture was stirred for 1 h at rt and quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by C18 (Column: XSelect CSH Prep C18 OBD, 30×150 mm, 5 m; Mobile Phase A: Water (0.1% FA), Mobile Phase B: ACN; Flow rate: 60 mL/min mL/min; Gradient (B %): 30 to 60% B in 10 min; Wave Length: 254 nm/220 nm; RT1(min): 9.2) to provide 2-chloro-N—((S)-1-((S)-1-((3R,5′S)-5′-cyano-2-oxospiro[indoline-3,3′-pyrrolidin]-1′-yl)-3-cyclopropyl-1-oxopropan-2-yl)-2-oxoazocan-3-yl)-2,2-difluoroacetamide (69.4 mg, 43%) as a white solid. ¹H NMR (400 MHz, 80° C., DMSO-d₆) δ 10.40 (br, 1H), 8.34-8.98 (m, 1H), 7.06-7.20 (m, 1H), 6.94-7.05 (m, 1H), 6.92-6.93 (m, 1H), 6.72-6.91 (m, 1H), 4.94-5.20 (m, 2H), 4.62-4.80 (m, 1H), 3.80-4.01 (m, 2H), 3.36-3.70 (m, 2H), 2.50-2.58 (m, 1H), 2.40-2.47 (m, 1H), 1.35-1.70 (m, 9H), 1.03-1.29 (m, 1H), 0.41-0.63 (m, 1H), 0.18-0.35 (m, 2H), −0.10-0.05 (m, 2H). LC-MS (ESI, m/z): 584 [M+Na]⁺.

### Example 23

### Compounds 23A and 23B

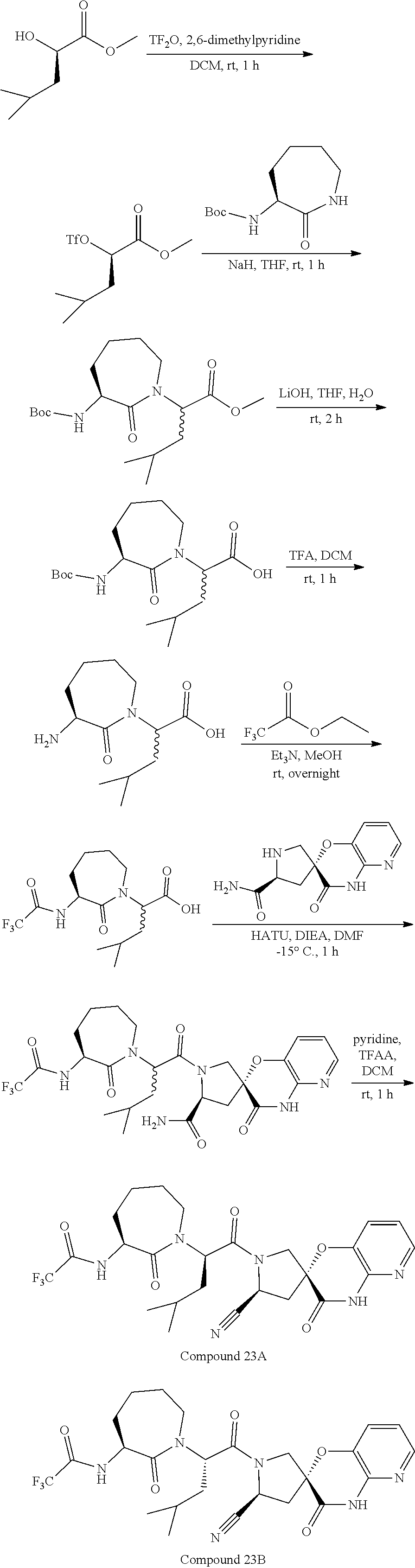

To a mixture of methyl (R)-2-hydroxy-4-methylpentanoate (480 mg, 3.28 mmol) in DCM (15 mL) was added 2,6-dimethylpyridine (703 mg, 6.56 mmol) and trifluoromethanesulfonic anhydride (1.67 g, 5.90 mmol) at 0° C. The mixture was stirred for 1 h at rt and diluted with MTBE (60 mN. The mixture was washed with brine:HCl (3:1, 3×20 mL), dried over anhydrous sodium sulfate, filtered and concentrated under reduced pressure to afford methyl (R)-4-methyl-2-(((trifluoromethyl)sulfonyl)oxy)pentanoate (914 mg, crude) as a light brown oil.

To a mixture of t-butyl (S)-(2-oxoazepan-3-yl)carbamate (500 mg, 2.19 mmol) in THF (10 mL) was added NaH (113 mg, 2.84 mmol, 60% in oil) at 0° C. After stirring for 0.5 h at 0° C., methyl (R)-4-methyl-2-(((trifluoromethyl)sulfonyl)oxy)pentanoate (914 mg, 3.28 mmol) was added. The mixture was stirred for 1 h at rt and the reaction was quenched with water (30 mL). The mixture was extracted with EtOAc (3×30 mL). The organic layers were combined, washed with brine (2×30 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl 2-((S)-3-((tert-butoxycarbonyl)amino)-2-oxoazepan-1-yl)-4-methylpentanoate (430 mg, 55%) as a yellow oil. ¹H NMR (400 MHz, DMSO-d₆) δ 6.53-6.63 (m, 1H), 5.09-5.20 (m, 1H), 4.24-4.34 (m, 1H), 3.63 (s, 3H), 3.26-3.44 (m, 2H), 1.54-1.84 (m, 7H), 1.41-1.46 (m, 2H), 1.36-1.40 (m, 9H), 0.79-0.90 (m, 6H). LC-MS (ESI, m/z): 357 [M+H]⁺.

To a mixture of methyl 2-((S)-3-((tert-butoxycarbonyl)amino)-2-oxoazepan-1-yl)-4-methylpentanoate (430 mg, 1.20 mmol) in THF (5 mL) was added a solution of LiOH (87.0 mg, 3.61 mmol) in H₂O (5 mL). The mixture was stirred for 2 h at rt and the reaction was quenched with water (10 mL). The mixture was acidified to pH=3 with HCl (1 M). The mixture was extracted with EtOAc (3×10 mL). The organic layers were combined, washed with brine (2×10 mL), dried over anhydrous sodium sulfate, filtered and concentrated under reduced pressure to afford 2-((S)-3-((tert-butoxycarbonyl)amino)-2-oxoazepan-1-yl)-4-methylpentanoic acid (380 mg, crude) as a yellow oil. ¹H NMR (400 MHz, DMSO-d₆) δ 12.65 (br, 1H), 6.50-6.58 (m, 1H), 5.03-5.12 (m, 1H), 4.23-4.30 (m, 1H), 3.25-3.36 (m, 2H), 1.53-1.82 (m, 9H), 1.36-1.40 (m, 9H), 0.87-0.90 (m, 3H), 0.78-0.89 (m, 3H). LC-MS (ESI, m/z): 365 [M+Na]⁺.

To a mixture of 2-((S)-3-((tert-butoxycarbonyl)amino)-2-oxoazepan-1-yl)-4-methylpentanoic acid (350 mg, 1.02 mmol) in DCM (10 mL) was added TFA (3 mL). The mixture was stirred for 1 h at rt and concentrated under reduced pressure to afford 2-((S)-3-amino-2-oxoazepan-1-yl)-4-methylpentanoic acid (247 mg, crude) as a yellow semi-solid. LC-MS (ESI, m/z): 243 [M+H]⁺.

To a mixture of 2-((S)-3-amino-2-oxoazepan-1-yl)-4-methylpentanoic acid (247 mg, 1.01 mmol) in MeOH (5 mL) were added triethylamine (1.23 g, 12.2 mmol) and ethyl 2,2,2-trifluoroacetate (1.44 g, 10.1 mmol). The mixture was stirred overnight, and the reaction was quenched with water (10 mL), then concentrated under reduced pressure to remove MeOH and acidified to pH=3 with HCl (1 M). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide 4-methyl-2-((S)-2-oxo-3-(2,2,2-trifluoroacetamido)azepan-1-yl)pentanoic acid (200 mg, 52%) as a light yellow solid. ¹H NMR (400 MHz, DMSO-d₆) δ 12.62 (br, 1H), 9.37-9.43 (m, 1H), 4.99-5.05 (m, 1H), 4.60-4.67 (m, 1H), 3.35-3.46 (m, 2H), 1.77-1.91 (m, 3H), 1.53-1.76 (m, 6H), 0.86-0.93 (m, 3H), 0.77-0.85 (m, 3H). LC-MS (ESI, m/z): 339 [M+H]⁺.

To a mixture of t-butyl (2R,5′S)-5′-carbamoyl-3-oxo-3,4-dihydrospiro[pyrido[3,2-b][1,4]oxazine-2,3′-pyrrolidine]-1′-carboxylate (100 mg, 0.287 mmol) in DCM (3 mL) was added TFA (1 mL). The mixture was stirred for 1 h at rt and concentrated under reduced pressure to afford (2R,5′S)-3-oxo-3,4-dihydrospiro[pyrido[3,2-b][1,4]oxazine-2,3′-pyrrolidine]-5′-carboxamide (71.0 mg, crude) as a yellow semi-solid. LC-MS (ESI, m/z): 249 [M+H]⁺.

To a mixture of 4-methyl-2-((S)-2-oxo-3-(2,2,2-trifluoroacetamido)azepan-1-yl)pentanoic acid (100 mg, 0.296 mmol), (2R,5′S)-3-oxo-3,4-dihydrospiro[pyrido[3,2-b][1,4]oxazine-2,3′-pyrrolidine]-5′-carboxamide (73.0 mg, 0.296 mmol) and HATU (134 mg, 0.355 mmol) in DMF (3 mL) was added N-ethyl-N-isopropylpropan-2-amine (152 mg, 1.18 mmol) at −15° C. The mixture was stirred for 1 h at −15° C. and the reaction was quenched with water (10 mL). The mixture was extracted with EtOAc (3×10 mL). The organic layers were combined, washed with brine (2×10 mL), dried over anhydrous sodium sulfate, filtered and concentrated under reduced pressure to afford the crude product. The crude product was purified by TLC (Mobile phase: MeOH:DCM=1:10; Rf=0.5; detection: UV) to provide (2R,5′S)-1′-(4-methyl-2-((S)-2-oxo-3-(2,2,2-trifluoroacetamido)azepan-1-yl)pentanoyl)-3-oxo-3,4-dihydrospiro[pyrido[3,2-b][1,4]oxazine-2,3′-pyrrolidine]-5′-carboxamide (90.0 mg, 53%) as a light yellow solid. ¹H NMR (400 MHz, DMSO-d₆) δ 11.64 (s, 1H), 9.38-9.47 (m, 1H), 7.98-8.02 (m, 1H), 7.49 (s, 1H), 7.19-7.24 (m, 1H), 7.00-7.08 (m, 2H), 5.16-5.27 (m, 1H), 4.61-4.70 (m, 1H), 4.26-4.35 (m, 1H), 4.17-4.23 (m, 1H), 3.97-4.05 (m, 1H), 3.34-3.52 (m, 2H), 2.46-2.49 (m, 1H), 1.98-2.07 (m, 1H), 1.65-1.89 (m, 4H), 1.35-1.57 (m, 5H), 0.85-0.90 (m, 6H). LC-MS (ESI, m/z): 591 [M+Na]⁺.

To a mixture of (2R,5′S)-1′-(4-methyl-2-((S)-2-oxo-3-(2,2,2-trifluoroacetamido)azepan-1-yl)pentanoyl)-3-oxo-3,4-dihydrospiro[pyrido[3,2-b][1,4]oxazine-2,3′-pyrrolidine]-5′-carboxamide (90.0 mg, 0.158 mmol) in DCM (3 mL) was added pyridine (62.0 mg, 0.790 mmol) and trifluoroacetic anhydride (66.0 mg, 0.316 mmol). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was separated by prep-Chiral-HPLC (Column: CHIRALPAK IG, 2×25 cm, 5 m; Mobile Phase A: Hex (0.1% FA), Mobile Phase B: EtOH:DCM=1:1; Flow rate: 20 mL/min; Gradient: isocratic 40; Wave Length: 254/220 nm). Purification resulted compound 23A: N—((S)-1-((R)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazepan-3-yl)-2,2,2-trifluoroacetamide (7.2 mg, 8%) as a white solid. ¹H NMR (400 MHz, 80° C., DMSO-d₆) δ 11.34 (s, 1H), 9.01 (br, 1H), 7.90-8.05 (m, 1H), 7.25-7.40 (m, 1H), 6.90-7.10 (m, 1H), 5.11-5.25 (m, 1H), 4.90-5.10 (m, 1H), 4.60-4.75 (m, 1H), 3.70-3.88 (m, 2H), 3.40-3.60 (m, 1H), 3.15-3.35 (m, 1H), 2.70-2.90 (m, 2H), 1.84-2.00 (m, 3H), 1.68-1.83 (m, 2H), 1.40-1.67 (m, 3H), 1.20-1.35 (m, 1H), 0.90-0.98 (m, 3H), 0.81-0.89 (m, 3H). LC-MS (ESI, m/z): 573 [M+Na]⁺, and compound 23B: N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxoazepan-3-yl)-2,2,2-trifluoroacetamide (42.5 mg, 48%) as a white solid.

¹H NMR (400 MHz, 80° C., DMSO-d₆) δ 11.48 (s, 1H), 9.00 (s, 1H), 7.95-8.05 (m, 1H), 7.20-7.30 (m, 1H), 6.95-7.10 (m, 1H), 5.15-5.35 (m, 1H), 4.80-5.03 (m, 1H), 4.60-4.71 (m, 1H), 4.20-4.36 (m, 1H), 4.00-4.18 (m, 1H), 3.50-3.64 (m, 1H), 3.30-3.49 (m, 1H), 2.78-2.90 (m, 1H), 2.50-2.65 (m, 1H), 1.70-1.94 (m, 4H), 1.54-1.69 (m, 2H), 1.40-1.53 (m, 2H), 1.20-1.39 (m, 1H), 0.90-0.97 (m, 3H), 0.80-0.89 (m, 3H). LC-MS (ESI, m/z): 573 [M+Na]⁺.

### Example 24

### Compound 24

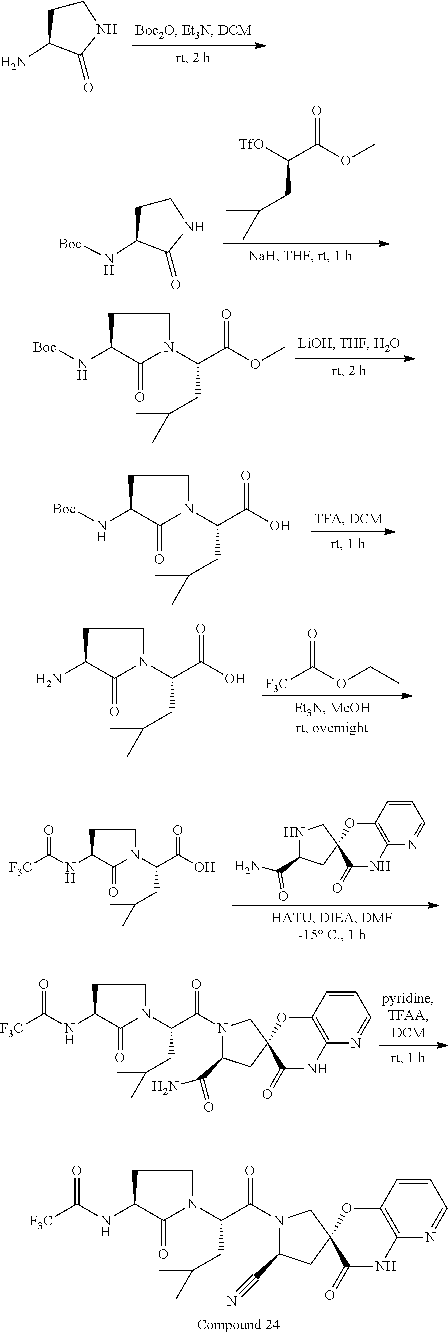

To a mixture of (S)-3-aminopyrrolidin-2-one (500 mg, 4.99 mmol) and di-tert-butyl dicarbonate (1.64 g, 7.49 mmol) in DCM (5 mL) was added triethylamine (1.01 g, 9.98 mmol) at rt. The mixture was stirred for 2 h at rt and concentrated under reduced pressure to afford the crude product. The crude product was purified by prep-HPLC (Column: XSelect CSH Prep C18 OBD, 30×150 mm, 5 m; Mobile Phase A: Water (0.1% FA), Mobile Phase B: ACN; Flow rate: 60 mL/min; Gradient: 11 to 41% B in 7 min; Wave Length: 254 nm/220 nm; RT1(min): 6.58) to provide tert-butyl (S)-(2-oxopyrrolidin-3-yl)carbamate (560 mg, 50%) as a white solid. ¹H NMR (400 MHz, DMSO-d₆) δ 7.70 (s, 1H), 6.79-7.12 (m, 1H), 3.87-4.16 (m, 1H), 3.04-3.18 (m, 2H), 2.14-2.27 (m, 1H), 1.72-1.91 (m, 1H), 1.39 (s, 9H). LC-MS (ESI, m/z): 145 [M−56+H]⁺.

To a solution of tert-butyl (S)-(2-oxopyrrolidin-3-yl)carbamate (550 mg, 2.74 mmol) in THF (15 mL) was added NaH (143 mg, 3.57 mmol, 60% in oil) at 0° C. After stirring for 1 h at rt, a solution of methyl (R)-4-methyl-2-(((trifluoromethyl)sulfonyl)oxy)pentanoate (1.15 g, 4.12 mmol) in THF (2 mL) was added dropwise at 0° C. The mixture was stirred for 1 h at rt and the reaction was quenched with water (50 mL). The mixture was extracted with EtOAc (3×50 mL). The organic layers were combined, washed with brine (2×50 mL), dried over anhydrous sodium sulfate, filtered and concentrated under reduced pressure to afford the crude product. The crude product was purified by C18 column with CH₃CN/Water (0.05% TFA). The fraction was concentrated under reduced pressure to provide methyl (S)-2-((S)-3-((tert-butoxycarbonyl)amino)-2-oxopyrrolidin-1-yl)-4-methylpentanoate (320 mg, 28%) as a light brown oil. ¹H NMR (400 MHz, DMSO-d₆) δ 7.02-7.27 (m, 1H), 4.51-4.69 (m, 1H), 4.12-4.28 (m, 1H), 3.64 (s, 3H), 3.14-3.36 (m, 2H), 2.16-2.30 (m, 1H), 1.66-1.86 (m, 2H), 1.46-1.62 (m, 2H), 1.33-1.40 (m, 9H), 0.81-0.91 (m, 6H). LC-MS (ESI, m/z): 273 [M−56+H]⁺.

To a solution of methyl (S)-2-((S)-3-((tert-butoxycarbonyl)amino)-2-oxopyrrolidin-1-yl)-4-methylpentanoate (320 mg, 0.974 mmol) in THF (2 mL) was added a solution of LiOH (71.0 mg, 2.92 mmol) and water (2 mL). The mixture was stirred for 2 h at rt and water (20 mL) was added. The mixture was adjusted to pH=6 with HCl (1 M). The mixture was extracted with EtOAc (3×20 mL). The organic layers were combined, washed with brine (2×20 mL), dried over anhydrous sodium sulfate, filtered and concentrated under reduced pressure to afford (S)-2-((S)-3-((tert-butoxycarbonyl)amino)-2-oxopyrrolidin-1-yl)-4-methylpentanoic acid (200 mg, crude) as a light yellow semi-solid. LC-MS (ESI, m/z): 259 [M−56+H]⁺.

To a mixture of (S)-2-((S)-3-((tert-butoxycarbonyl)amino)-2-oxopyrrolidin-1-yl)-4-methylpentanoic acid (200 mg, 0.636 mmol) in DCM (6 mL) was added TFA (2 mL) at 0° C. The mixture was stirred for 1 h at rt and concentrated under reduced pressure to afford (S)-2-((S)-3-amino-2-oxopyrrolidin-1-yl)-4-methylpentanoic acid (140 mg, crude) as a light-yellow oil. LC-MS (ESI, m/z): 215 [M+H]⁺.

To a mixture of (S)-2-((S)-3-amino-2-oxopyrrolidin-1-yl)-4-methylpentanoic acid (140 mg, 0.636 mmol) in MeOH (2 mL) was added triethylamine (770 mg, 7.63 mmol) and ethyl 2,2,2-trifluoroacetate (929 mg, 6.36 mmol). The mixture was stirred overnight at rt and the reaction was quenched with water (10 mL). The mixture was acidified to pH=6 with HCl (1 M), then extracted with EtOAc (3×10 mL). The organic layers were combined, washed with brine (2×10 mL), dried over anhydrous sodium sulfate, filtered and concentrated under reduced pressure to afford (S)-4-methyl-2-((S)-2-oxo-3-(2,2,2-trifluoroacetamido)pyrrolidin-1-yl)pentanoic acid (200 mg, crude) as a light yellow semi-solid. ¹HNMR (400 MHz, DMSO-d₆) δ 12.85 (br, 1H), 9.63-9.85 (m, 1H), 4.57-4.72 (m, 1H), 4.45-4.54 (m, 1H), 3.43-3.50 (m, 1H), 3.24-3.28 (m, 1H), 2.26-2.34 (m, 1H), 1.65-1.74 (m, 1H), 1.55-1.61 (m, 1H), 1.41-1.52 (m, 2H), 0.82-0.92 (m, 6H). LC-MS (ESI, m/z): 311 [M+H]⁺.

To a mixture of (S)-4-methyl-2-((S)-2-oxo-3-(2,2,2-trifluoroacetamido)pyrrolidin-1-yl)pentanoic acid (120 mg, 0.387 mmol), (2R,5′S)-3-oxo-3,4-dihydrospiro[pyrido[3,2-b][1,4]oxazine-2,3′-pyrrolidine]-5′-carboxamide (97.0 mg, 0.387 mmol) and HATU (177 mg, 0.464 mmol) in DMF (3 mL) was added N-ethyl-N-isopropylpropan-2-amine (150 mg, 1.16 mmol) at −15° C. The mixture was stirred for 1 h at −15° C. and the reaction was quenched with water (10 mL). The mixture was extracted with EtOAc (3×10 mL). The organic layers were combined, washed with brine (2×10 mL), dried over anhydrous sodium sulfate, filtered and concentrated under reduced pressure to remove the solvent. The crude product was purified by TLC (Mobile phase: MeOH:DCM=1:11; Rf=0.4; detection: UV) to provide (2R,5′S)-1′-((S)-4-methyl-2-((S)-2-oxo-3-(2,2,2-trifluoroacetamido)pyrrolidin-1-yl)pentanoyl)-3-oxo-3,4-dihydrospiro[pyrido[3,2-b][1,4]oxazine-2,3′-pyrrolidine]-5′-carboxamide (110 mg, 46%) as a light yellow solid. ¹H NMR (400 MHz, DMSO-d₆) δ 11.58 (s, 1H), 9.62-9.78 (m, 1H), 7.91-8.01 (m, 1H), 7.43-7.54 (m, 1H), 7.28-7.39 (m, 1H), 6.93-7.09 (m, 2H), 4.64-4.72 (m, 1H), 4.50-4.61 (m, 1H), 4.32-4.43 (m, 1H), 4.03-4.18 (m, 2H), 3.53-3.62 (m, 1H), 3.23-3.31 (m, 1H), 2.42-2.49 (m, 1H), 2.18-2.34 (m, 2H), 1.87-1.95 (m, 1H), 1.43-1.65 (m, 3H), 0.79-0.94 (m, 6H). LC-MS (ESI, m/z): 541 [M+H]⁺.

To a mixture of (2R,5′S)-1′-((S)-4-methyl-2-((S)-2-oxo-3-(2,2,2-trifluoroacetamido)pyrrolidin-1-yl)pentanoyl)-3-oxo-3,4-dihydrospiro[pyrido[3,2-b][1,4]oxazine-2,3′-pyrrolidine]-5′-carboxamide (110 mg, 0.204 mmol) in DCM (2 mL) was added pyridine (81.0 mg, 1.02 mmol) and trifluoroacetic anhydride (86.0 mg, 0.408 mmol). The mixture was stirred for 1 h at rt and the reaction was quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was purified by prep-HPLC (Column: XSelect CSH Prep C18 OBD, 30×150 mm, 5 m; Mobile Phase A: Water (0.05% TFA), Mobile Phase B: ACN; Flow rate: 60 mL/min; Gradient: 25 to 55% B in 7 min; Wave Length: 254 nm/220 nm; RT1(min): 6.1) to provide N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxopyrrolidin-3-yl)-2,2,2-trifluoroacetamide (60.1 mg, 56%) as a white solid. ¹H NMR (400 MHz, 80° C., DMSO-d₆) δ 11.40 (s, 1H), 9.47 (s, 1H), 7.90-8.00 (m, 1H), 7.25-7.38 (m, 1H), 6.90-7.03 (m, 1H), 4.90-5.10 (m, 1H), 4.62-4.80 (m, 1H), 4.40-4.61 (m, 1H), 4.05-4.25 (m, 2H), 3.50-3.65 (m, 1H), 3.25-3.45 (m, 1H), 2.77-2.87 (m, 1H), 2.65-2.76 (m, 1H), 2.27-2.40 (m, 1H), 1.90-2.10 (m, 1H), 1.58-1.75 (m, 2H), 1.40-1.57 (m, 1H), 0.92-1.00 (m, 3H), 0.78-0.91 (m, 3H). LC-MS (ESI, m/z): 523 [M+H]⁺.

### Example 25

### Compounds 25 and 25-1

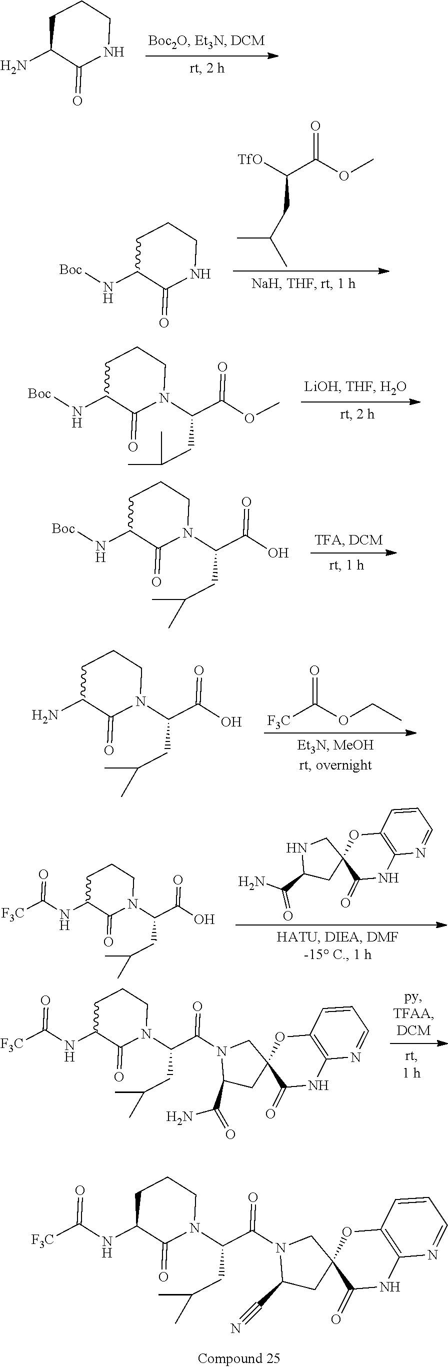

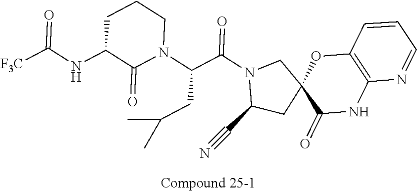 [SMILES: CC(C)C[C@@H](C(=O)N1C[C@@]2(C[C@H]1C#N)Oc1cccnc1NC2=O)N1CCC[C@@H](NC(=O)C(F)(F)F)C1=O]

To a mixture of (S)-3-aminopiperidin-2-one (500 mg, 4.38 mmol) and di-tert-butyl dicarbonate (1.44 g, 6.57 mmol) in DCM (5 mL) was added triethylamine (887 mg, 8.76 mmol) at rt. The mixture was stirred for 2 h at rt and concentrated under reduced pressure to afford the crude product. The crude product was purified by prep-HPLC (Column: WelFlash C18, Regular C18 20-40 m, 330 g; Mobile Phase A: Water (0.1% FA), Mobile Phase B: ACN; Flow rate: 100 mL/min; Gradient: 2 to 32% B in 30 min; Wave Length: 254 nm/220 nm; RT1(min): 28.68) to provide tert-butyl (S)-(2-oxopiperidin-3-yl)carbamate (600 mg, 57%) as a light yellow semi-solid. ¹H NMR (400 MHz, DMSO-d₆) δ 7.48 (m, 1H), 6.75-6.85 (m, 1H), 3.73-3.83 (m, 1H), 3.07-3.13 (m, 2H), 1.83-1.94 (m, 1H), 1.63-1.80 (m, 3H), 1.33-1.37 (m, 9H). LC-MS (ESI, m/z): 159 [M−56+H]⁺.

To a solution of t-butyl (S)-(2-oxopiperidin-3-yl)carbamate (600 mg, 2.80 mmol) in THF (10 mL) was added NaH (146 mg, 3.64 mmol, 60% in oil) at 0° C. After stirring for 1 h at rt, a solution of methyl (R)-4-methyl-2-(((trifluoromethyl)sulfonyl)oxy)pentanoate (1.17 g, 4.20 mmol) in THF (2 mL) was added dropwise at 0° C. The mixture was stirred for 1 h at rt and the reaction was quenched with water (40 mL). The mixture was extracted with EtOAc (3×50 mL). The organic layers were combined, washed with brine (2×50 mL), dried over anhydrous sodium sulfate, filtered and concentrated under reduced pressure to afford the crude product. The crude product was purified by C18 column with CH₃CN/Water(0.05% TFA). The fraction was concentrated under reduced pressure to provide methyl (2S)-2-(3-((tert-butoxycarbonyl)amino)-2-oxopiperidin-1-yl)-4-methylpentanoate (420 mg, 39%) as a brown oil. ¹H NMR (400 MHz, DMSO-d₆) δ 6.75-7.07 (m, 1H), 4.86-5.08 (m, 1H), 3.97-4.07 (m, 1H), 3.57-3.65 (m, 3H), 3.12-3.29 (m, 2H), 1.50-2.03 (m, 7H), 1.24-1.44 (m, 9H), 0.84-0.93 (m, 6H). LC-MS (ESI, m/z): 343 [M+H]⁺.

To a solution of methyl (2S)-2-(3-((tert-butoxycarbonyl)amino)-2-oxopiperidin-1-yl)-4-methylpentanoate (420 mg, 1.22 mmol) in THF (3 mL) was added a solution of LiOH (89.0 mg, 3.68 mmol) in water (3 mL). The mixture was stirred for 2 h at rt and the reaction was quenched with water (20 mL). The mixture was adjusted to pH=6 with HCl (1 M). The mixture was extracted with EtOAc (3×20 mL). The organic layers were combined, washed with brine (2×20 mL), dried over anhydrous sodium sulfate, filtered and concentrated under reduced pressure to afford (2S)-2-(3-((tert-butoxycarbonyl)amino)-2-oxopiperidin-1-yl)-4-methylpentanoic acid (340 mg, 84%) as a light yellow semi-solid. ¹H NMR (400 MHz, DMSO-d₆) δ 12.63 (br, 1H), 6.69-7.12 (m, 1H), 4.72-5.14 (m, 1H), 3.87-4.03 (m, 1H), 3.09-3.28 (m, 2H), 1.45-1.98 (m, 7H), 1.32-1.41 (m, 9H), 0.83-0.91 (m, 6H). LC-MS (ESI, m/z): 329 [M+H]⁺.

To a mixture of (2S)-2-(3-((tert-butoxycarbonyl)amino)-2-oxopiperidin-1-yl)-4-methylpentanoic acid (340 mg, 1.03 mmol) in DCM (9 mL) was added TFA (3 mL) at 0° C. The mixture was stirred for 1 h at rt and concentrated under reduced pressure to afford (2S)-2-(3-amino-2-oxopiperidin-1-yl)-4-methylpentanoic acid (240 mg, crude) as a brown oil. LC-MS (ESI, m/z): 229 [M+H]⁺.

To a mixture of (2S)-2-(3-amino-2-oxopiperidin-1-yl)-4-methylpentanoic acid (240 mg, 1.03 mmol) in MeOH (5 mL) was added triethylamine (1.28 g, 12.4 mmol) and ethyl 2,2,2-trifluoroacetate (1.50 g, 10.3 mmol). The mixture was stirred overnight at rt and the reaction was quenched with water (10 mL). The mixture was adjusted to pH=6 with HCl (1 M) and extracted with EtOAc (3×30 mL). The organic layers were combined, washed with brine (2×30 mL), dried over anhydrous sodium sulfate, filtered and concentrated under reduced pressure to afford (2S)-4-methyl-2-(2-oxo-3-(2,2,2-trifluoroacetamido)piperidin-1-yl)pentanoic acid (300 mg, crude) as a yellow semi-solid. ¹H NMR (400 MHz, DMSO-d₆) δ 12.65 (br, 1H), 9.49-9.72 (m, 1H), 4.88-5.03 (m, 1H), 4.24-4.48 (m, 1H), 3.06-3.12 (m, 2H), 1.40-1.95 (m, 7H), 0.83-0.92 (m, 6H). LC-MS (ESI, m/z): 325 [M+H]⁺.

To a mixture of (2S)-4-methyl-2-(2-oxo-3-(2,2,2-trifluoroacetamido)piperidin-1-yl)pentanoic acid (260 mg, 0.802 mmol), (2R,5′S)-3-oxo-3,4-dihydrospiro[pyrido[3,2-b][1,4]oxazine-2,3′-pyrrolidine]-5′-carboxamide (200 mg, 0.802 mmol) and HATU (366 mg, 0.962 mmol) in DMF (5 mL) was added N-ethyl-N-isopropylpropan-2-amine (311 mg, 2.40 mmol) at −15° C. The mixture was stirred for 1 h at −15° C. and the reaction was quenched with water (20 mL). The mixture was extracted with EtOAc (3×20 mL). The organic layers were combined, washed with brine (2×20 mL), dried over anhydrous sodium sulfate, filtered and concentrated under reduced pressure to afford the crude product. The crude product was purified by TLC (Mobile phase: MeOH:DCM=1:11; Rf=0.3; detection: UV) to provide (2R,5′S)-1′-((2S)-4-methyl-2-(2-oxo-3-(2,2,2-trifluoroacetamido)piperidin-1-yl)pentanoyl)-3-oxo-3,4-dihydrospiro[pyrido[3,2-b][1,4]oxazine-2,3′-pyrrolidine]-5′-carboxamide (75.0 mg, 14%) as a light yellow solid. ¹H NMR (400 MHz, DMSO-d₆) δ 11.43-11.65 (m, 1H), 9.37-9.70 (m, 1H), 7.89-8.02 (m, 1H), 7.22-7.51 (m, 2H), 6.88-7.09 (m, 2H), 4.96-5.21 (m, 1H), 4.24-4.48 (m, 2H), 3.89-4.13 (m, 2H), 3.35-3.42 (m, 1H), 3.23-3.30 (m, 1H), 2.41-2.48 (m, 1H), 2.13-2.29 (m, 1H), 1.46-1.94 (m, 7H), 0.84-0.92 (m, 6H). LC-MS (ESI, m/z): 577 [M+Na]⁺.

To a mixture of (2R,5′S)-1′-((2S)-4-methyl-2-(2-oxo-3-(2,2,2-trifluoroacetamido)piperidin-1-yl)pentanoyl)-3-oxo-3,4-dihydrospiro[pyrido[3,2-b][1,4]oxazine-2,3′-pyrrolidine]-5′-carboxamide (75.0 mg, 0.135 mmol) in DCM (2 mL) was added pyridine (54.0 mg, 0.675 mmol) and trifluoroacetic anhydride (57.0 mg, 0.270 mmol). The mixture was stirred for 1 h at rt and quenched with water (10 mL). The mixture was extracted with DCM (3×10 mL). The organic layers were combined, washed with brine (2×10 mL), dried over anhydrous sodium sulfate, filtered and concentrated under reduced pressure to afford the crude product. The crude product was separated by Chiral-HPLC column (CHIRALPAK AD-H, 2×25 cm, 5 m; Mobile Phase A: Hex-HPLC, Mobile Phase B: IPA; Flow rate: 20 mL/min; Gradient: isocratic 30; Wavelength: 254/220 nm). Purification resulted in compound 25: N—((S)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxopiperidin-3-yl)-2,2,2-trifluoroacetamide (24.6 mg, 33%) as a white solid. ¹H NMR (400 MHz, 80° C., DMSO-d₆) δ 11.40 (s, 1H), 9.25 (br, 1H), 7.90-8.00 (m, 1H), 7.20-7.35 (m, 1H), 6.90-7.02 (m, 1H), 5.00-5.10 (m, 1H), 4.85-4.99 (m, 1H), 4.25-4.35 (m, 1H), 4.05-4.20 (m, 2H), 3.38-3.48 (m, 1H), 3.25-3.37 (m, 1H), 2.75-2.85 (m, 1H), 2.62-2.74 (m, 1H), 1.95-2.05 (m, 1H), 1.88-1.94 (m, 1H), 1.70-1.87 (m, 2H), 1.58-1.69 (m, 2H), 1.42-1.57 (m, 1H), 0.80-0.98 (m, 6H). LC-MS (ESI, m/z): 559 [M+Na]⁺, and compound 25-1: N—((R)-1-((S)-1-((2R,5′S)-5′-cyano-3-oxo-3,4-dihydrospiro[pyrido[3,2-b][1,4]oxazine-2,3′-pyrrolidin]-1′-yl)-4-methyl-1-oxopentan-2-yl)-2-oxopiperidin-3-yl)-2,2,2-trifluoroacetamide (7.9 mg, 10%) as a white solid. ¹H NMR (400 MHz, 80° C., DMSO-d₆) δ 11.40 (s, 1H), 9.30 (s, 1H), 7.90-8.00 (m, 1H), 7.25-7.40 (m, 1H), 6.90-7.05 (m, 1H), 5.10-5.28 (m, 1H), 4.90-5.09 (m, 1H), 3.95-4.20 (m, 3H), 3.31-3.50 (m, 1H), 3.20-3.30 (m, 1H), 2.65-2.90 (m, 2H), 1.95-2.07 (m, 1H), 1.85-1.94 (m, 1H), 1.75-1.84 (m, 2H), 1.62-1.72 (m, 1H), 1.45-1.61 (m, 2H), 0.80-1.00 (m, 6H). LC-MS (ESI, m/z): 559 [M+Na]⁺.

### Example 26

### Additional Compounds

Table 1 includes compounds that were prepared using procedures analogous to those described above.

TABLE 1

| Cmpd | | |
|---|---|---|
| No. | Structure | LC-MS |
| 26 | 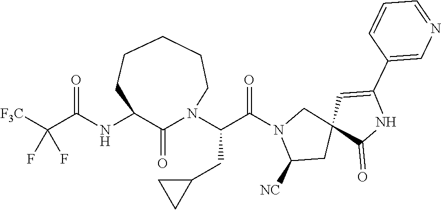 [SMILES: N#C[C@@H]1C[C@@]2(C=C(c3cccnc3)NC2=O)CN1C(=O)[C@H](CC1CC1)N1CCCCC[C@H](NC(=O)C(F)(F)C(F)(F)F)C1=O] | 645 [M + Na]⁺ |
| 27 | 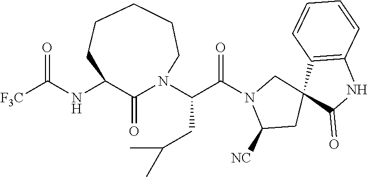 [SMILES: CC(C)C[C@@H](C(=O)N1C[C@]2(C[C@H]1C#N)C(=O)Nc1ccccc12)N1CCCCC[C@H](NC(=O)C(F)(F)F)C1=O] | 570 [M + Na]⁺ |
| 28 | 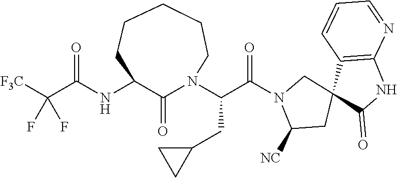 [SMILES: N#C[C@@H]1C[C@@]2(CN1C(=O)[C@H](CC1CC1)N1CCCCC[C@H](NC(=O)C(F)(F)C(F)(F)F)C1=O)C(=O)Nc1ncccc12] | 619 [M + Na]⁺ |
| 29 | 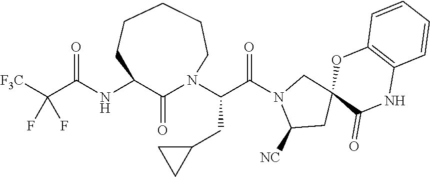 [SMILES: N#C[C@@H]1C[C@]2(CN1C(=O)[C@H](CC1CC1)N1CCCCC[C@H](NC(=O)C(F)(F)C(F)(F)F)C1=O)Oc1ccccc1NC2=O] | 634 [M + Na]⁺ |
| 30 | 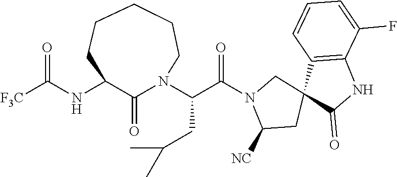 [SMILES: CC(C)C[C@@H](C(=O)N1C[C@]2(C[C@H]1C#N)C(=O)Nc1c(F)cccc12)N1CCCCC[C@H](NC(=O)C(F)(F)F)C1=O] | 588 [M + Na]⁺ |
| 31 | 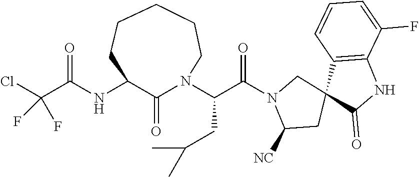 [SMILES: CC(C)C[C@@H](C(=O)N1C[C@]2(C[C@H]1C#N)C(=O)Nc1c(F)cccc12)N1CCCCC[C@H](NC(=O)C(F)(F)Cl)C1=O] | 604 [M + Na]⁺ |
| 32 | 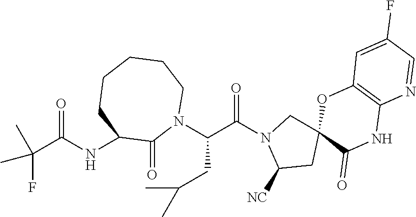 [SMILES: CC(C)C[C@@H](C(=O)N1C[C@@]2(C[C@H]1C#N)Oc1cc(F)cnc1NC2=O)N1CCCCC[C@H](NC(=O)C(C)(C)F)C1=O] | 597 [M + Na]⁺ |
| 33 | 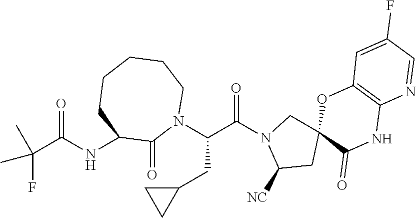 [SMILES: CC(C)(F)C(=O)N[C@H]1CCCCCN([C@@H](CC2CC2)C(=O)N2C[C@@]3(C[C@H]2C#N)Oc2cc(F)cnc2NC3=O)C1=O] | 595 [M + Na]⁺ |
| 34 | 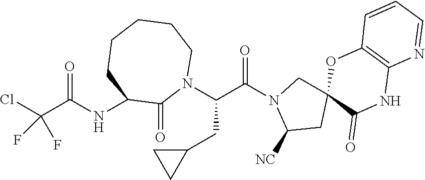 [SMILES: N#C[C@@H]1C[C@]2(CN1C(=O)[C@H](CC1CC1)N1CCCCC[C@H](NC(=O)C(F)(F)Cl)C1=O)Oc1cccnc1NC2=O] | 601 [M + Na]⁺ |
| 35 | 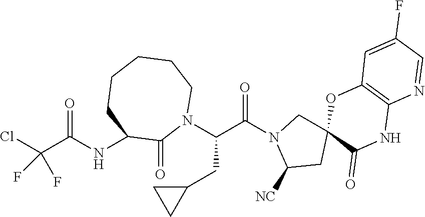 [SMILES: N#C[C@@H]1C[C@]2(CN1C(=O)[C@H](CC1CC1)N1CCCCC[C@H](NC(=O)C(F)(F)Cl)C1=O)Oc1cc(F)cnc1NC2=O] | 321 (fragment peak) |
| 36 | 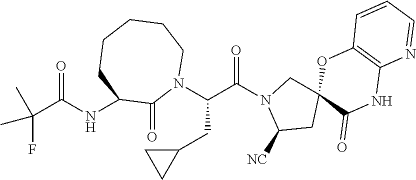 [SMILES: CC(C)(F)C(=O)N[C@H]1CCCCCN([C@@H](CC2CC2)C(=O)N2C[C@@]3(C[C@H]2C#N)Oc2cccnc2NC3=O)C1=O] | 577 [M + Na]⁺ |
| 37 | 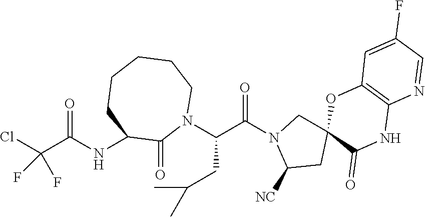 [SMILES: CC(C)C[C@@H](C(=O)N1C[C@@]2(C[C@H]1C#N)Oc1cc(F)cnc1NC2=O)N1CCCCC[C@H](NC(=O)C(F)(F)Cl)C1=O] | 621 [M + Na]⁺ |
| 38 | 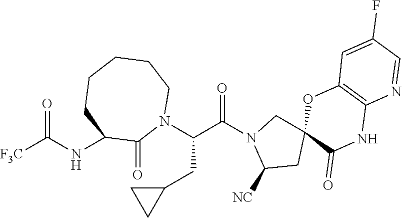 [SMILES: N#C[C@@H]1C[C@]2(CN1C(=O)[C@H](CC1CC1)N1CCCCC[C@H](NC(=O)C(F)(F)F)C1=O)Oc1cc(F)cnc1NC2=O] | 305 (fragment peak) |
| 39 | 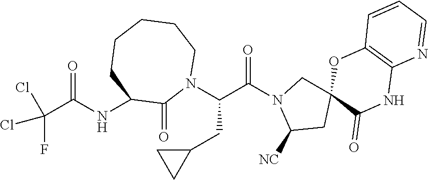 [SMILES: N#C[C@@H]1C[C@]2(CN1C(=O)[C@H](CC1CC1)N1CCCCC[C@H](NC(=O)C(F)(Cl)Cl)C1=O)Oc1cccnc1NC2=O] | 617 [M + Na]⁺ |
| 40 | 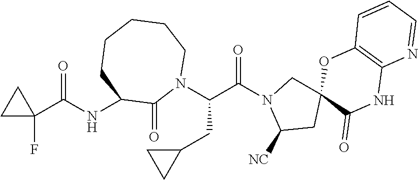 [SMILES: N#C[C@@H]1C[C@]2(CN1C(=O)[C@H](CC1CC1)N1CCCCC[C@H](NC(=O)C3(F)CC3)C1=O)Oc1cccnc1NC2=O] | 575 [M + Na]⁺ |
| 41 | 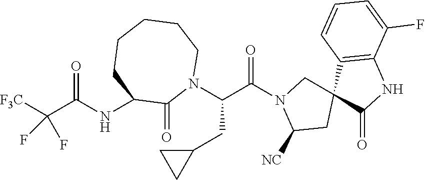 [SMILES: N#C[C@@H]1C[C@@]2(CN1C(=O)[C@H](CC1CC1)N1CCCCC[C@H](NC(=O)C(F)(F)C(F)(F)F)C1=O)C(=O)Nc1c(F)cccc12] | 636 [M + Na]⁺ |
| 42 | 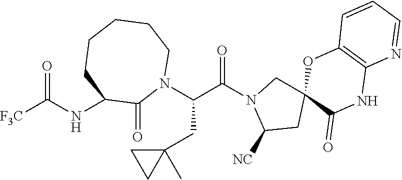 [SMILES: CC1(C[C@@H](C(=O)N2C[C@@]3(C[C@H]2C#N)Oc2cccnc2NC3=O)N2CCCCC[C@H](NC(=O)C(F)(F)F)C2=O)CC1] | 577 [M + Na]⁺ |
| 43 | 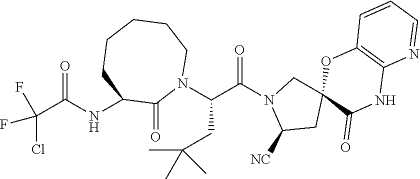 [SMILES: CC(C)(C)C[C@@H](C(=O)N1C[C@@]2(C[C@H]1C#N)Oc1cccnc1NC2=O)N1CCCCC[C@H](NC(=O)C(F)(F)Cl)C1=O] | 617 [M + Na]⁺ |
| 44 | 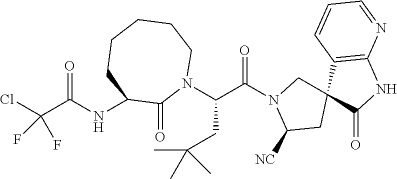 [SMILES: CC(C)(C)C[C@@H](C(=O)N1C[C@]2(C[C@H]1C#N)C(=O)Nc1ncccc12)N1CCCCC[C@H](NC(=O)C(F)(F)Cl)C1=O] | 579 [M + Na]⁺ |
| 45 | 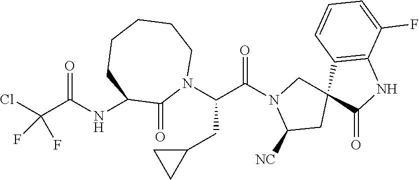 [SMILES: N#C[C@@H]1C[C@@]2(CN1C(=O)[C@H](CC1CC1)N1CCCCC[C@H](NC(=O)C(F)(F)Cl)C1=O)C(=O)Nc1c(F)cccc12] | 602 [M + Na]⁺ |
| 46 | 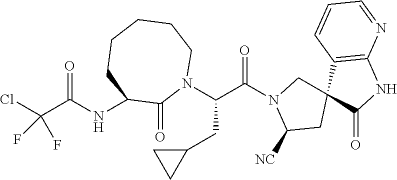 [SMILES: N#C[C@@H]1C[C@@]2(CN1C(=O)[C@H](CC1CC1)N1CCCCC[C@H](NC(=O)C(F)(F)Cl)C1=O)C(=O)Nc1ncccc12] | 585 [M + Na]⁺ |
| 47 | 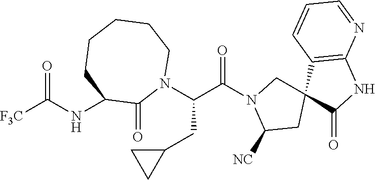 [SMILES: N#C[C@@H]1C[C@@]2(CN1C(=O)[C@H](CC1CC1)N1CCCCC[C@H](NC(=O)C(F)(F)F)C1=O)C(=O)Nc1ncccc12] | 547 [M + Na]⁺ |
| 48 | 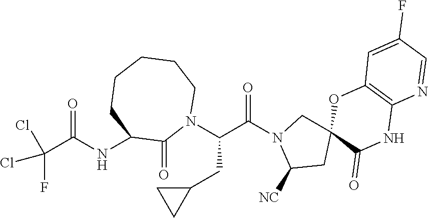 [SMILES: N#C[C@@H]1C[C@]2(CN1C(=O)[C@H](CC1CC1)N1CCCCC[C@H](NC(=O)C(F)(Cl)Cl)C1=O)Oc1cc(F)cnc1NC2=O] | 635 [M + Na]⁺ |
| 49 | 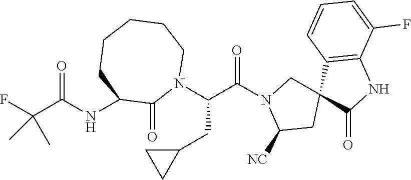 [SMILES: CC(C)(F)C(=O)N[C@H]1CCCCCN([C@@H](CC2CC2)C(=O)N2C[C@]3(C[C@H]2C#N)C(=O)Nc2c(F)cccc23)C1=O] | 556 [M + Na]⁺ |
| 50 | 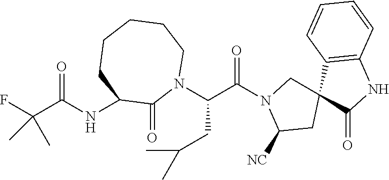 [SMILES: CC(C)C[C@@H](C(=O)N1C[C@]2(C[C@H]1C#N)C(=O)Nc1ccccc12)N1CCCCC[C@H](NC(=O)C(C)(C)F)C1=O] | 562 [M + Na]⁺ |
| 51 | 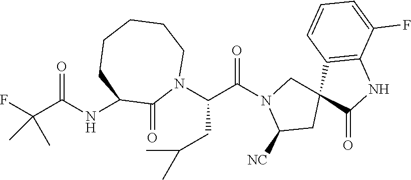 [SMILES: CC(C)C[C@@H](C(=O)N1C[C@]2(C[C@H]1C#N)C(=O)Nc1c(F)cccc12)N1CCCCC[C@H](NC(=O)C(C)(C)F)C1=O] | 580 [M + Na]⁺ |
| 52 | 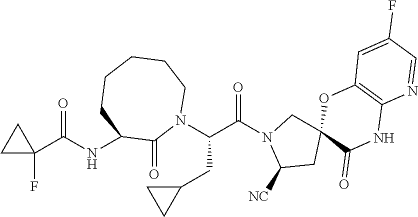 [SMILES: N#C[C@@H]1C[C@]2(CN1C(=O)[C@H](CC1CC1)N1CCCCC[C@H](NC(=O)C3(F)CC3)C1=O)Oc1cc(F)cnc1NC2=O] | 593 [M + Na]⁺ |
| 53 | 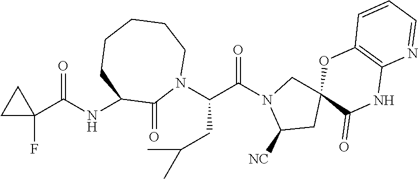 [SMILES: CC(C)C[C@@H](C(=O)N1C[C@@]2(C[C@H]1C#N)Oc1cccnc1NC2=O)N1CCCCC[C@H](NC(=O)C2(F)CC2)C1=O] | 577 [M + Na]⁺ |
| 54 | 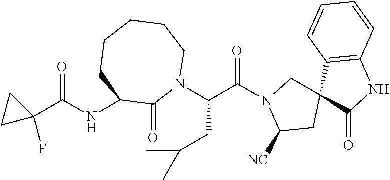 [SMILES: CC(C)C[C@@H](C(=O)N1C[C@]2(C[C@H]1C#N)C(=O)Nc1ccccc12)N1CCCCC[C@H](NC(=O)C2(F)CC2)C1=O] | 560 [M + Na]⁺ |
| 55 | 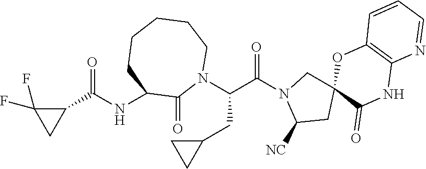 [SMILES: N#C[C@@H]1C[C@]2(CN1C(=O)[C@H](CC1CC1)N1CCCCC[C@H](NC(=O)[C@@H]3CC3(F)F)C1=O)Oc1cccnc1NC2=O] | 593 [M + Na]⁺ |
| 56 | 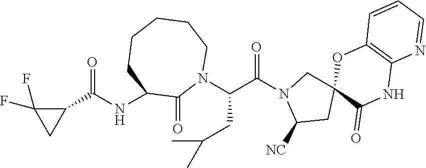 [SMILES: CC(C)C[C@@H](C(=O)N1C[C@@]2(C[C@H]1C#N)Oc1cccnc1NC2=O)N1CCCCC[C@H](NC(=O)[C@@H]2CC2(F)F)C1=O] | 595 [M + Na]⁺ |
| 57 | 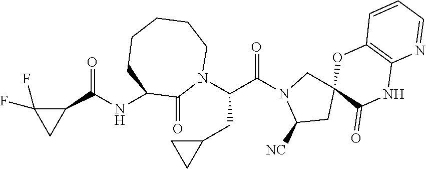 [SMILES: N#C[C@@H]1C[C@]2(CN1C(=O)[C@H](CC1CC1)N1CCCCC[C@H](NC(=O)[C@H]3CC3(F)F)C1=O)Oc1cccnc1NC2=O] | 593 [M + Na]⁺ |
| 58 | 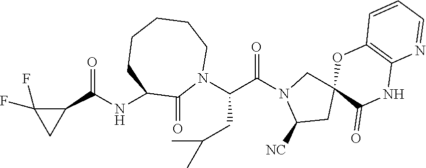 [SMILES: CC(C)C[C@@H](C(=O)N1C[C@@]2(C[C@H]1C#N)Oc1cccnc1NC2=O)N1CCCCC[C@H](NC(=O)[C@H]2CC2(F)F)C1=O] | 573 [M + Na]⁺ |
| 59 | 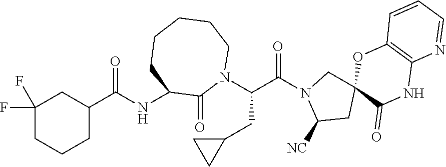 [SMILES: N#C[C@@H]1C[C@]2(CN1C(=O)[C@H](CC1CC1)N1CCCCC[C@H](NC(=O)C3CCCC(F)(F)C3)C1=O)Oc1cccnc1NC2=O] | 635 [M + Na]⁺ |
| 60 | 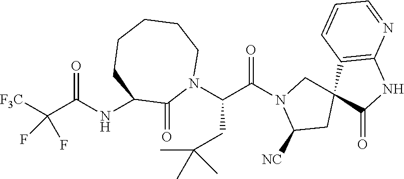 [SMILES: CC(C)(C)C[C@@H](C(=O)N1C[C@]2(C[C@H]1C#N)C(=O)Nc1ncccc12)N1CCCCC[C@H](NC(=O)C(F)(F)C(F)(F)F)C1=O] | 635 [M + Na]⁺ |
| 61 | 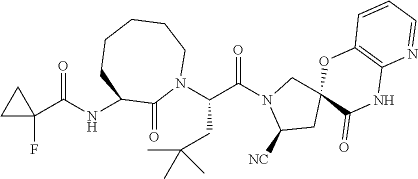 [SMILES: CC(C)(C)C[C@@H](C(=O)N1C[C@@]2(C[C@H]1C#N)Oc1cccnc1NC2=O)N1CCCCC[C@H](NC(=O)C2(F)CC2)C1=O] | 591 [M + Na]⁺ |
| 62 | 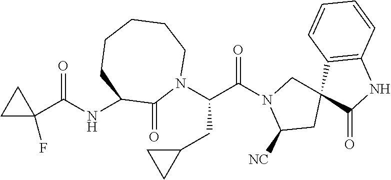 [SMILES: N#C[C@@H]1C[C@@]2(CN1C(=O)[C@H](CC1CC1)N1CCCCC[C@H](NC(=O)C3(F)CC3)C1=O)C(=O)Nc1ccccc12] | 558 [M + Na]⁺ |
| 63 | 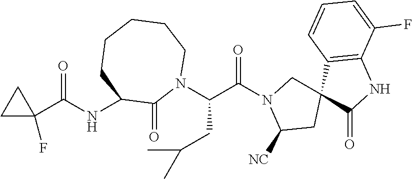 [SMILES: CC(C)C[C@@H](C(=O)N1C[C@]2(C[C@H]1C#N)C(=O)Nc1c(F)cccc12)N1CCCCC[C@H](NC(=O)C2(F)CC2)C1=O] | 556 [M + H]+ |
| 64 | 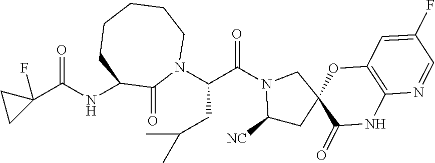 [SMILES: CC(C)C[C@@H](C(=O)N1C[C@@]2(C[C@H]1C#N)Oc1cc(F)cnc1NC2=O)N1CCCCC[C@H](NC(=O)C2(F)CC2)C1=O] | 595 [M + Na]⁺ |
| 65 | 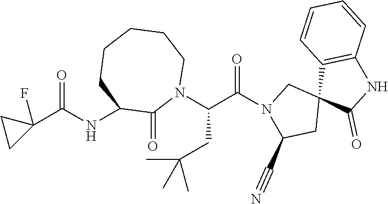 [SMILES: CC(C)(C)C[C@@H](C(=O)N1C[C@]2(C[C@H]1C#N)C(=O)Nc1ccccc12)N1CCCCC[C@H](NC(=O)C2(F)CC2)C1=O] | 574 [M + Na]⁺ |
| 66 | 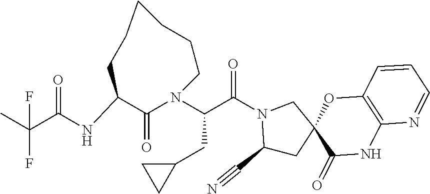 [SMILES: CC(F)(F)C(=O)N[C@H]1CCCCCCN([C@@H](CC2CC2)C(=O)N2C[C@@]3(C[C@H]2C#N)Oc2cccnc2NC3=O)C1=O] | 591 [M + Na]⁺ |
| 67 | 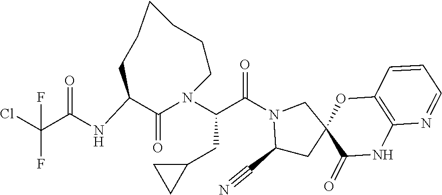 [SMILES: N#C[C@@H]1C[C@]2(CN1C(=O)[C@H](CC1CC1)N1CCCCCC[C@H](NC(=O)C(F)(F)Cl)C1=O)Oc1cccnc1NC2=O] | 615 [M + Na]⁺ |
| 68 | 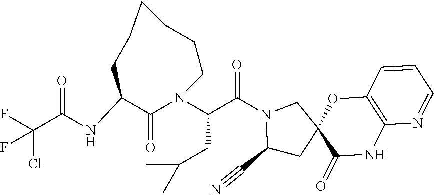 [SMILES: CC(C)C[C@@H](C(=O)N1C[C@@]2(C[C@H]1C#N)Oc1cccnc1NC2=O)N1CCCCCC[C@H](NC(=O)C(F)(F)Cl)C1=O] | 617 [M + Na]⁺ |
| 69 | 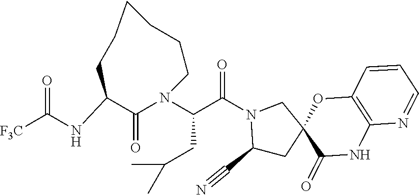 [SMILES: CC(C)C[C@@H](C(=O)N1C[C@@]2(C[C@H]1C#N)Oc1cccnc1NC2=O)N1CCCCCC[C@H](NC(=O)C(F)(F)F)C1=O] | 601 [M + Na]⁺ |
| 70 | 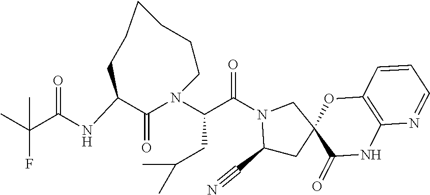 [SMILES: CC(C)C[C@@H](C(=O)N1C[C@@]2(C[C@H]1C#N)Oc1cccnc1NC2=O)N1CCCCCC[C@H](NC(=O)C(C)(C)F)C1=O] | 341 (fragment peak) |
| 71 | 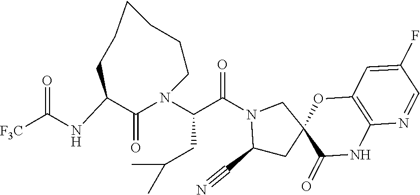 [SMILES: CC(C)C[C@@H](C(=O)N1C[C@@]2(C[C@H]1C#N)Oc1cc(F)cnc1NC2=O)N1CCCCCC[C@H](NC(=O)C(F)(F)F)C1=O] | 619 [M + Na]⁺ |
| 72 | 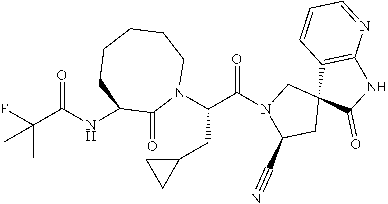 [SMILES: CC(C)(F)C(=O)N[C@H]1CCCCCN([C@@H](CC2CC2)C(=O)N2C[C@]3(C[C@H]2C#N)C(=O)Nc2ncccc23)C1=O] | 561 [M + Na]⁺ |
| 73 | 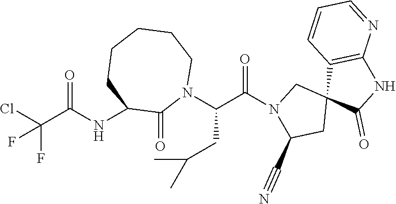 [SMILES: CC(C)C[C@@H](C(=O)N1C[C@]2(C[C@H]1C#N)C(=O)Nc1ncccc12)N1CCCCC[C@H](NC(=O)C(F)(F)Cl)C1=O] | 565 [M + Na]⁺ |
| 74 | 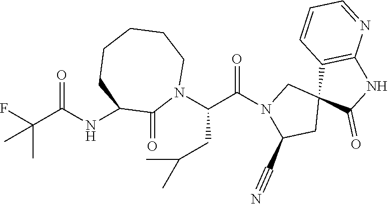 [SMILES: CC(C)C[C@@H](C(=O)N1C[C@]2(C[C@H]1C#N)C(=O)Nc1ncccc12)N1CCCCC[C@H](NC(=O)C(C)(C)F)C1=O] | 327 (fragment peak) |
| 75 | 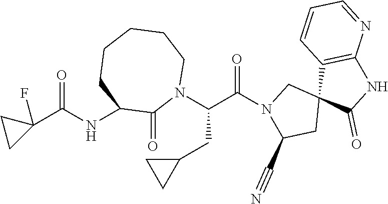 [SMILES: N#C[C@@H]1C[C@@]2(CN1C(=O)[C@H](CC1CC1)N1CCCCC[C@H](NC(=O)C3(F)CC3)C1=O)C(=O)Nc1ncccc12] | 323 (fragment peak) |
| 76 | 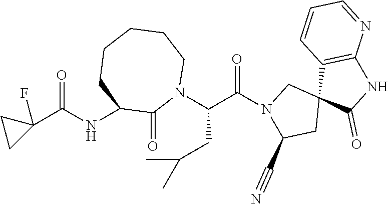 [SMILES: CC(C)C[C@@H](C(=O)N1C[C@]2(C[C@H]1C#N)C(=O)Nc1ncccc12)N1CCCCC[C@H](NC(=O)C2(F)CC2)C1=O] | 325 (fragment peak) |
| 77 | 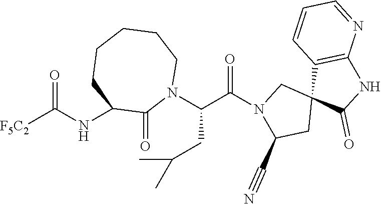 | 599 [M + Na]⁺ |
| 78 | 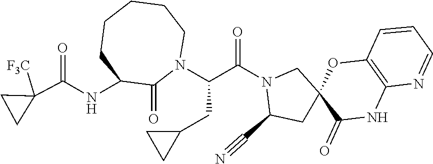 [SMILES: N#C[C@@H]1C[C@]2(CN1C(=O)[C@H](CC1CC1)N1CCCCC[C@H](NC(=O)C3(C(F)(F)F)CC3)C1=O)Oc1cccnc1NC2=O] | 625 [M + Na]⁺ |
| 79 | 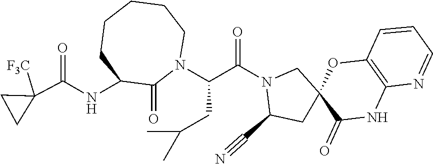 [SMILES: CC(C)C[C@@H](C(=O)N1C[C@@]2(C[C@H]1C#N)Oc1cccnc1NC2=O)N1CCCCC[C@H](NC(=O)C2(C(F)(F)F)CC2)C1=O] | 375 (fragment peak) |
| 80 | 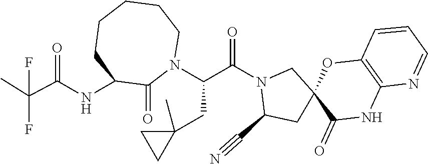 [SMILES: CC1(C[C@@H](C(=O)N2C[C@@]3(C[C@H]2C#N)Oc2cccnc2NC3=O)N2CCCCC[C@H](NC(=O)C(C)(F)F)C2=O)CC1] | 311 (fragment peak) |
| 81 | 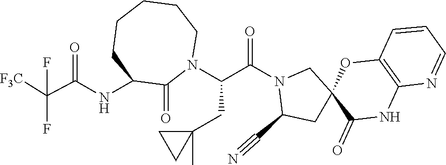 [SMILES: CC1(C[C@@H](C(=O)N2C[C@@]3(C[C@H]2C#N)Oc2cccnc2NC3=O)N2CCCCC[C@H](NC(=O)C(F)(F)C(F)(F)F)C2=O)CC1] | 341 (fragment peak) |

| Cmpd | | |
|---|---|---|
| No. | LC-MS | ¹H NMR |
| 26 | 645 [M + Na]⁺ | (400 MHz, DMSO-d₆) δ 9.62-9.79 (m, 1H), 8.63-8.81 (m, 1H), 8.41-8.62 (m, 1H), 7.80-8.01 (m, 2H), 7.58-7.79 (m, 1H), 7.35- 7.53 (m, 1H), 5.19-5.32 (m, 1H), 4.97-5.10 (m, 1H), 4.70-4.96 (m, 1H), 4.19-4.29 (m, 1H), 3.93-4.04 (m, 1H), 3.61-3.68 (m, 1H), 3.51-3.55 (m, 1H), 2.71-2.89 (m, 2H), 1.05-1.91 (m, 10H), 0.48-0.61 (m, 1H), 0.20-0.41 (m, 2H), 0.01-0.12 (m, 2H). |
| 27 | 570 [M + Na]⁺ | (400 MHz, 80º C, DMSO-d₆) δ 10.49 (s, 1H), 9.10-9.30 (m, 1H), 7.19-7.30 (m, 1H), 7.00-7.11 (m, 1H), 6.84-6.99 (m, 2H), 5.20-5.33 (m, 1H), 5.03-5.19 (m, 1H), 4.71-4.85 (m, 1H), 3.85- 4.00 (m, 2H), 3.48-3.71 (m, 2H), 2.60-2.69 (m, 1H), 2.51-2.58 (m, 1H), 1.42-1.80 (m, 10H), 1.11-1.25 (m, 1H), 0.88-0.95 (m, 3H), 0.73-0.87 (m, 3H). |
| 28 | 619 [M + Na]⁺ | (400 MHz, 80° C., DMSO-d₆) δ 11.11 (s, 1H), 9.15-9.25 (m, 1H), 8.05-8.15 (m, 1H), 7.40-7.50 (m, 1H), 6.90-7.02 (m, 1H), 5.04-5.28 (m, 2H), 4.78-4.99 (m, 1H), 3.95-4.10 (m, 2H), 3.61- 3.74 (m, 1H), 3.50-3.60 (m, 1H), 2.62-2.76 (m, 1H), 2.51-2.61 (m, 1H), 1.48-1.82 (m, 9H), 1.20-1.32 (m, 1H), 0.58-0.70 (m, 1H), 0.30-0.43 (m, 2H), 0.05-0.16 (m, 2H). |
| 29 | 634 [M + Na]⁺. | (400 MHz, 60° C., DMSO-d₆) δ 10.97 (s, 1H), 9.36 (s, 1H), 6.92-7.10 (m, 3H), 6.80-6.91 (m, 1H), 5.18-5.38 (m, 1H), 4.75- 5.05 (m, 2H), 4.25-4.35 (m, 1H), 4.12-4.24 (m, 1H), 3.60-3.80 (m, 1H), 3.50-3.59 (m, 1H), 2.70-2.85 (m, 1H), 2.52-2.68 (m, 1H), 1.66-1.85 (m, 3H), 1.40-1.65 (m, 7H), 0.51-0.68 (m, 1H), 0.32-0.46 (m, 1H), 0.20-0.32 (m, 1H), 0.03-0.16 (m, 2H). |
| 30 | 588 [M + Na]⁺. | (400 MHz, 80° C, DMSO-d₆) δ 11.04 (br, 1H), 9.18 (s, 1H), 7.09-7.25 (m, 1H), 6.86-7.08 (m, 2H), 5.21-5.37 (m, 1H), 5.10- 5.20 (m, 1H), 4.75-4.91 (m, 1H), 3.90-4.09 (m, 2H), 3.53-3.79 (m, 2H), 2.66-2.78 (m, 1H), 2.55-2.61 (m, 1H), 1.45-1.85 (m, 10H), 1.10-1.31 (m, 1H), 0.86-1.00 (m, 3H), 0.79-0.85 (m, 3H). |
| 31 | 604 [M + Na]⁺ | (400 MHz, 80° C, DMSO-d₆) δ 11.05 (br, 1H), 8.96 (s, 1H), 7.08-7.25 (m, 1H), 6.80-7.07 (m, 2H), 5.10-5.32 (m, 2H), 4.70- 4.90 (m, 1H), 3.90-4.09 (m, 2H), 3.50-3.78 (m, 2H), 2.67-2.73 (m, 1H), 2.55-2.60 (m, 1H), 1.45-1.80 (m, 10H), 1.10-1.30 (m, 1H), 0.87-1.00 (m, 3H), 0.77-0.86 (m, 3H). |
| 32 | 597 [M + Na]⁺ | (400 MHz, 60° C, DMSO-d₆) δ 7.90-8.05 (m, 1H), 7.41-7.65 (m, 1H), 7.11-7.30 (m, 1H), 5.12-5.33 (m, 1H), 4.88-5.02 (m, 1H), 4.68-4.87 (m, 1H), 4.12-4.35 (m, 2H), 3.60-3.78 (m, 1H), 3.49-3.58 (m, 1H), 2.80-2.86 (m, 1H), 2.63-2.71 (m, 1H), 1.71- 1.85 (m, 1H), 1.31-1.70 (m, 16H), 0.85-0.92 (m, 3H), 0.75-0.84 (m, 3H). |
| 33 | 595 [M + Na]⁺ | (400 MHz, DMSO-d₆) δ 11.80 (br, 1H), 7.90-8.15 (m, 1H), 7.50-7.78 (m, 1H), 7.18-7.45 (m, 1H), 5.05-5.20 (m, 1H), 4.85- 5.00 (m, 1H), 4.70-4.84 (m, 1H), 4.26-4.32 (m, 1H), 4.11-4.25 (m, 1H), 3.61-3.70 (m, 1H), 3.41-3.50 (m, 1H), 2.78-2.91 (m, 1H), 2.68-2.75 (m, 1H), 1.30-1.87 (m, 16H), 0.51-0.70 (m, 1H), 0.20-0.45 (m, 2H), 0.01-0.16 (m, 2H). |
| 34 | 601 [M + Na]⁺ | (400 MHz, 80° C, DMSO-d₆) δ 8.83 (br, 1H), 7.81-8.48 (m, 1H), 7.08-7.28 (m, 1H), 6.86-6.98 (m, 1H), 5.00-5.18 (m, 1H), 4.78-4.90 (m, 1H), 4.65-4.77 (m, 1H), 4.09-4.24 (m, 2H), 3.51- 3.61 (m, 1H), 3.38-3.50 (m, 1H), 2.68-2.72 (m, 1H), 2.54-2.67 (m, 1H), 1.31-1.81 (m, 10H), 0.41-0.57 (m, 1H), 0.18-0.37 (m, 2H), -0.03-0.06 (m, 2H). |
| 35 | 321 (fragment peak) | (400 MHz, 80° C, DMSO-d₆) δ 11.60 (br, 0.18H), 8.70-9.01 (m, 1H), 7.90-8.09 (m, 1H), 7.17-7.33 (m, 1H), 5.10-5.21 (m, 1H), 4.88-4.99 (m, 1H), 4.75-4.87 (m, 1H), 4.13-4.31 (m, 2H), 3.60-3.70 (m, 1H), 3.49-3.59 (m, 1H), 2.80-2.86 (m, 1H), 2.68- 2.72 (m, 1H), 1.40-1.85 (m, 10H), 0.52-0.78 (m, 1H), 0.30-0.46 (m, 2H), 0.01-0.11 (m, 2H). |
| 36 | 577 [M + Na]⁺ | (400 MHz, DMSO-d₆) δ 11.70 (br, 1H), 7.90-8.11 (m, 1H), 7.59-7.80 (m, 1H), 7.21-7.40 (m, 1H), 6.95-7.10 (m, 1H), 5.01- 5.22 (m, 1H), 4.87-5.00 (m, 1H), 4.65-4.86 (m, 1H), 4.22-4.31 (m, 1H), 4.11-4.21 (m, 1H), 3.58-3.72 (m, 1H), 3.42-3.48 (m, 1H), 2.77-2.90 (m, 1H), 2.60-2.76 (m, 1H), 1.31-1.85 (m, 16H), 0.49-0.70 (m, 1H), 0.15-0.41 (m, 2H), 0.01-0.12 (m, 2H). |
| 37 | 621 [M + Na]⁺ | (400 MHz, 80° C, DMSO-d₆) δ 11.35 (br, 1H), 8.96 (br, 1H), 7.90-8.10 (m, 1H), 7.20-7.31 (m, 1H), 5.20-5.30 (m, 1H), 4.86- 5.00 (m, 1H), 4.71-4.85 (m, 1H), 4.15-4.32 (m, 2H), 3.59-3.72 (m, 1H), 3.48-3.58 (m, 1H), 2.75-2.88 (m, 1H), 2.60-2.73 (m, 1H), 1.65-1.85 (m, 2H), 1.52-1.64 (m, 7H), 1.33-1.51 (m, 2H), 0.85-0.92 (m, 3H), 0.75-0.84 (m, 3H). |
| 38 | 305 (fragment peak) | (400 MHz, 80° C, DMSO-d₆) δ 11.59 (br, 1H), 9.12 (s, 1H), 7.95-8.05 (m, 1H), 7.20-7.35 (m, 1H), 5.10-5.31 (m, 1H), 4.80- 5.08 (m, 2H), 4.10-4.35 (m, 2H), 3.60-3.78 (m, 1H), 3.50-3.59 (m, 1H), 2.80-2.90 (m, 1H), 2.70-2.79 (m, 1H), 1.42-1.90 (m, 10H), 0.50-0.71 (m, 1H), 0.27-0.50 (m, 2H), 0.03-0.20 (m, 2H). |
| 39 | 617 [M + Na]⁺ | (400 MHz, 80° C., DMSO-d₆) δ 11.43 (s, 1H), 8.55-8.70 (m, 1H), 7.90-8.05 (m, 1H), 7.24-7.35 (m, 1H), 6.90-7.10 (m, 1H), 5.10-5.30 (m, 1H), 4.90-5.07 (m, 1H), 4.73-4.89 (m, 1H), 4.11- 4.31 (m, 2H), 3.60-3.75 (m, 1H), 3.42-3.59 (m, 1H), 2.80-2.90 (m, 1H), 2.65-2.79 (m, 1H), 1.48-1.90 (m, 10H), 0.52-0.75 (m, 1H), 0.23-0.50 (m, 2H), 0.06-0.20 (m, 2H) |
| 40 | 575 [M + Na]+ | (400 MHz, 80° C, DMSO-d₆) δ 11.51 (br, 1H), 7.90-8.05 (m, 1H), 7.60-7.75 (m, 1H), 7.22-7.37 (m, 1H), 6.98-7.10 (m, 1H), 5.10-5.25 (m, 1H), 4.75-5.01 (m, 2H), 4.25-4.43 (m, 1H), 4.09- 4.24 (m, 1H), 3.61-3.70 (m, 1H), 3.45-3.60 (m, 1H), 2.68-2.89 (m, 2H), 1.77-1.92 (m, 1H), 1.46-1.76 (m, 9H), 1.26-1.35 (m, 2H), 1.05-1.25 (m, 2H), 0.55-0.70 (m, 1H), 0.30-0.45 (m, 2H), 0.05-0.14 (m, 2H). |
| 41 | 636 [M + Na]⁺ | (400 MHz, 80° C, DMSO-d₆) δ 11.00 (s, 1H), 9.19 (s, 1H), 7.08-7.22 (m, 1H), 6.89-7.04 (m, 2H), 5.00-5.30 (m, 2H), 4.70- 4.93 (m, 1H), 3.92-4.12 (m, 2H), 3.60-2.73 (m, 1H), 3.48-3.59 (m, 1H), 2.60-2.73 (m, 1H), 2.52-2.59 (m, 1H), 1.45-1.85 (m, 9H), 1.15-1.35 (m, 1H), 0.55-0.70 (m, 1H), 0.28-0.47 (m, 2H), 0.04-0.13 (m, 2H). |
| 42 | 577 [M + H]⁺ | (400 MHz, DMSO-d₆) δ 11.72 (br, 1H), 9.50-9.70 (m, 1H), 7.90-8.10 (m, 1H), 7.20-7.40 (m, 1H), 6.95-7.10 (m, 1H), 5.30- 5.50 (m, 1H), 4.75-5.01 (m, 2H), 4.16-4.35 (m, 2H), 3.60-3.70 (m, 1H), 3.45-3.55 (m, 1H), 2.76-2.84 (m, 1H), 2.62-2.69 (m, 1H), 1.81-1.92 (m, 1H), 1.64-1.77 (m, 2H), 1.53-1.63 (m, 3H), 1.42-1.52 (m, 3H), 1.31-1.41 (m, 1H), 0.92-1.01 (m, 3H), 0.19- 0.22 (m, 2H), 0.06-0.18 (m, 2H). |
| 43 | 617 [M + Na]⁺ | (400 MHz, 80° C, DMSO-d₆) δ 11.50 (br, 1H), 8.90-9.10 (m, 1H), 7.90-8.05 (m, 1H), 7.10-7.24 (m, 1H), 6.85-7.08 (m, 1H), 5.27-5.45 (m, 1H), 4.88-5.05 (m, 1H), 4.69-4.87 (m, 1H), 4.15- 4.35 (m, 2H), 3.50-3.75 (m, 2H), 2.72-2.85 (m, 1H), 2.60-2.71 (m, 1H), 2.08-2.18 (m, 1H), 1.62-1.80 (m, 2H), 1.55-1.61 (m, 3H), 1.47-1.54 (m, 2H), 1.38-1.46 (m, 1H), 1.29-1.37 (m, 1H), 0.75-0.90 (m, 9H). |
| 44 | 579 [M + H]⁺ | (400 MHz, 60° C., DMSO-d₆) δ 11.24 (br, 1H), 9.12 (s, 1H), 8.10-8.25 (m, 1H), 7.40-7.55 (m, 1H), 6.90-7.05 (m, 1H), 5.28- 5.40 (m, 1H), 5.08-5.27 (m, 1H), 4.70-4.90 (m, 1H), 3.85-4.10 (m, 2H), 3.50-3.72 (m, 2H), 2.68-2.80 (m, 1H), 2.55-2.65 (m, 1H), 2.10-2.20 (m, 1H), 1.40-1.80 (m, 7H), 1.25-1.39 (m, 1H), 0.96-1.11 (m, 1H), 0.76-0.95 (m, 9H). |
| 45 | 602 [M + Na]⁺ | (400 MHz, 80° C, DMSO-d₆) δ 11.00 (s, 1H), 8.85-9.00 (m, 1H), 7.10-7.20 (m, 1H), 6.88-7.01 (m, 2H), 5.01-5.30 (m, 2H), 4.70-4.88 (m, 1H), 3.90-4.10 (m, 2H), 3.60-3.80 (m, 1H), 3.47- 3.59 (m, 1H), 2.61-2.77 (m, 1H), 2.50-2.60 (m, 1H), 1.43-1.85 (m, 9H), 1.14-1.32 (m, 1H), 0.56-0.70 (m, 1H), 0.30-0.47 (m, 2H), 0.04-0.16 (m, 2H). |
| 46 | 585 [M + Na]⁺ | (400 MHz, 80° C., DMSO-d₆) δ 11.12 (br, 1H), 8.80-9.00 (m, 1H), 8.00-8.20 (m, 1H), 7.40-7.50 (m, 1H), 6.90-7.00 (m, 1H), 5.05-5.25 (m, 2H), 4.70-4.90 (m, 1H), 4.06-4.11 (m, 1H), 3.92- 4.05 (m, 1H), 3.60-3.72 (m, 1H), 3.50-3.58 (m, 1H), 2.65-2.75 (m, 1H), 2.51-2.60 (m, 1H), 1.73-1.85 (m, 2H), 1.63-1.72 (m, 2H), 1.45-1.62 (m, 5H), 1.20-1.40 (m, 1H), 0.56-0.73 (m, 1H), 0.28-0.45 (m, 2H), 0.05-0.15 (m, 2H). |
| 47 | 547 [M + H]⁺ | (400 MHz, DMSO-d₆) δ 11.38 (s, 1H), 9.45-9.70 (m, 1H), 8.00-8.21 (m, 1H), 7.40-7.60 (m, 1H), 6.85-7.10 (m, 1H), 5.14- 5.30 (m, 1H), 4.95-5.13 (m, 1H), 4.75-4.94 (m, 1H), 3.95-4.15 (m, 2H), 3.60-3.80 (m, 1H), 3.45-3.59 (m, 1H), 2.65-2.75 (m, 1H), 2.51-2.60 (m, 1H), 1.42-1.78 (m, 9H), 1.10-1.28 (m, 1H), 0.53-0.70 (m, 1H), 0.28-0.50 (m, 2H), 0.02-0.19 (m, 2H). |
| 48 | 635 [M + Na]⁺ | (400 MHz, 80° C, DMSO-d₆) δ 11.58 (br, 1H), 8.50-8.70 (m, 1H), 7.90-8.05 (m, 1H), 7.20-7.35 (m, 1H), 5.10-5.25 (m, 1H), 4.87-5.00 (m, 1H), 4.76-4.86 (m, 1H), 4.18-4.30 (m, 2H), 3.60- 3.72 (m, 1H), 3.48-3.59 (m, 1H), 2.78-2.90 (m, 1H), 2.66-2.77 (m, 1H), 1.78-1.90 (m, 1H), 1.40-1.77 (m, 9H), 0.52-0.67 (m, 1H), 0.29-0.34 (m, 2H), 0.01-0.11 (m, 2H). |
| 49 | 556 [M + H]⁺ | (400 MHz, 80° C, DMSO-d₆) δ 11.00 (s, 1H), 7.30-7.50 (m, 1H), 7.08-7.28 (m, 1H), 6.80-7.08 (m, 2H), 4.92-5.42 (m, 2H), 4.56-4.91 (m, 1H), 3.90-4.15 (m, 2H), 3.49-3.75 (m, 2H), 2.64- 2.79 (m, 1H), 2.50-2.63 (m, 1H), 1.15-1.90 (m, 16H), 0.60-0.78 (m, 1H), 0.27-0.52 (m, 2H), 0.05-0.25 (m, 2H). |
| 50 | 562 [M + Na]⁺ | (400 MHz, 80° C, DMSO- d6) δ 10.50 (br, 1H), 7.38-7.55 (m, 1H), 7.18-7.30 (m, 1H), 7.01-7.15 (m, 1H), 6.79-7.00 (m, 2H), 5.20-5.30 (m, 1H), 5.01-5.19 (m, 1H), 4.63-4.88 (m, 1H), 3.80- 4.05 (m, 2H), 3.50-3.78 (m, 2H), 2.60-2.72 (m, 1H), 2.53-2.59 (m, 1H), 1.35-1.85 (m, 16H), 1.15-1.29 (m, 1H), 0.88-0.95 (m, 3H), 0.78-0.87 (m, 3H). |
| 51 | 580 [M + Na]⁺ | (400 MHz, 80° C., DMSO-d₆) δ 10.99 (s, 1H), 7.38-7.58 (m, 1H), 7.05-7.27 (m, 1H), 6.90-7.03 (m, 2H), 5.20-5.30 (m, 1H), 5.09-5.19 (m, 1H), 4.70-4.87 (m, 1H), 3.91-4.03 (m, 2H), 3.49- 3.88 (m, 2H), 2.62-2.77 (m, 1H), 2.55-2.61 (m, 1H), 1.71-1.85 (m, 1H), 1.60-1.70 (m, 4H), 1.46-1.59 (m, 6H) 1.39-1.45 (m, 3H), 1.32-1.38 (m, 2H), 1.12-1.30 (m, 1H), 0.88-0.97 (m, 3H), 0.75-0.87 (m, 3H). |
| 52 | 593 [M + Na]⁺ | (400 MHz, 80° C, DMSO-d₆) δ 11.60 (br, 1H), 7.95-8.05 (m, 1H), 7.60-7.78 (m, 1H), 7.20-7.35 (m, 1H), 5.05-5.21 (m, 1H), 4.80-5.00 (m, 2H), 4.26-4.40 (m, 1H), 4.10-4.25 (m, 1H), 3.60- 3.75 (m, 1H), 3.41-3.59 (m, 1H), 2.80-2.90 (m, 1H), 2.70-2.79 (m, 1H), 1.40-1.90 (m, 10H), 1.20-1.30 (m, 2H), 1.03-1.19 (m, 2H), 0.50-0.70 (m, 1H), 0.30-0.40 (m, 2H), 0.00-0.10 (m, 2H). |
| 53 | 577 [M + Na]⁺ | (400 MHz, 80° C, DMSO-d₆) δ 11.41 (s, 1H), 7.92-8.04 (m, 1H), 7.62-7.80 (m, 1H), 7.20-7.30 (m, 1H), 6.92-7.10 (m, 1H), 5.20-5.31 (m, 1H), 4.85-5.01 (m, 2H), 4.18-4.37 (m, 2H), 3.60- 3.71 (m, 1H), 3.48-3.59 (m, 1H), 2.74-2.89 (m, 1H), 2.64-2.72 (m, 1H), 1.40-1.90 (m, 11H), 1.21-1.38 (m, 2H), 1.08-1.20 (m, 2H), 0.80-0.98 (m, 6H). |
| 54 | 560 [M + Na]⁺ | (400 MHz, 80° C., DMSO- d6) δ 10.53 (s, 1H), 7.65-7.83 (m, 1H), 7.20-7.35 (m, 1H), 7.05-7.15 (m, 1H), 6.86-7.02 (m, 2H), 5.22-5.32 (m, 1H), 5.10-5.21 (m, 1H), 4.75-4.95 (m, 1H), 3.88- 4.05 (m, 2H), 3.50-3.80 (m, 2H), 2.61-2.74 (m, 1H), 2.53-2.60 (m, 1H), 1.42-1.90 (m, 10H), 1.04-1.32 (m, 5H), 0.88-1.00 (m, 3H), 0.78-0.87 (m, 3H). |
| 55 | 593 [M + Na]⁺ | (400 MHz, 80° C, DMSO- d6) δ 11.41 (br, 1H), 8.20-8.32 (m, 1H), 7.90-8.04 (m, 1H), 7.19-7.35 (m, 1H), 6.90-7.05 (m, 1H), 5.10-5.25 (m, 1H), 4.70-5.00 (m, 2H), 4.10-4.35 (m, 2H), 3.56- 3.74 (m, 1H), 3.40-3.55 (m, 1H), 2.75-2.85 (m, 1H), 2.60-2.74 (m, 2H), 1.69-1.89 (m, 3H), 1.36-1.68 (m, 9H), 0.50-0.70 (m, 1H), 0.28-0.46 (m, 2H), 0.02-0.12 (m, 2H). |
| 56 | 595 [M + Na]⁺ | (400 MHz, 80° C., DMSO-d₆) δ 11.42 (br, 1H), 8.20-8.35 (m, 1H), 7.90-8.05 (m, 1H), 7.12-7.30 (m, 1H), 6.95-7.08 (m, 1H), 5.19-5.36 (m, 1H), 4.88-4.99 (m, 1H), 4.76-4.87 (m, 1H), 4.12- 4.30 (m, 2H), 3.60-3.70 (m, 1H), 3.45-3.59 (m, 1H), 2.73-2.86 (m, 1H), 2.60-2.72 (m, 2H), 1.35-1.90 (m, 13H), 0.86-0.95 (m, 3H), 0.75-0.85 (m, 3H). |
| 57 | 593 [M + Na]⁺ | (400 MHz, DMSO-d₆) δ 11.39 (br, 1H), 8.12-8.42 (m, 1H), 7.81-8.11 (m, 1H), 7.06-7.31 (m, 1H), 6.75-7.05 (m, 1H), 5.05- 5.26 (m, 1H), 4.78-4.97 (m, 2H), 4.10-4.31 (m, 2H), 3.52-3.67 (m, 1H), 3.32-3.51 (m, 1H), 2.71-2.80 (m, 1H), 2.57-2.70 (m, 2H), 1.71-1.87 (m, 1H), 1.31-1.70 (m, 11H), 0.42-0.60 (m, 1H), 0.21-0.37 (m, 2H), -0.02-0.07 (m, 2H). |
| 58 | 573 [M + H]⁺ | (400 MHz, 80° C., DMSO-d₆) δ 11.45 (br, 1H), 8.20-8.40 (m, 1H), 7.90-8.05 (m, 1H), 7.15-7.28 (m, 1H), 6.95-7.05 (m, 1H), 5.20-5.35 (m, 1H), 4.80-5.00 (m, 2H), 4.15-4.35 (m, 2H), 3.56- 3.72 (m, 1H), 3.45-3.55 (m, 1H), 2.75-2.85 (m, 1H), 2.58-2.74 (m, 2H), 1.80-1.90 (m, 1H), 1.65-1.79 (m, 2H), 1.40-1.64 (m, 9H), 1.30-1.39 (m, 1H), 0.84-0.90 (m, 3H), 0.72-0.83 (m, 3H). |
| 59 | 635 [M + Na]⁺ | (400 MHz, 80° C, DMSO- d6) δ 11.45 (br, 1H), 7.90-8.05 (m, 1H), 7.70-7.89 (m, 1H), 7.14-7.34 (m, 1H), 6.90-7.10 (m, 1H), 5.12-5.22 (m, 1H), 4.85-4.98 (m, 1H), 4.70-4.84 (m, 1H), 4.10- 4.30 (m, 2H), 3.58-3.70 (m, 1H), 3.43-3.57 (m, 1H), 2.75-2.82 (m, 1H), 2.65-2.74 (m, 1H), 2.45-2.49 (m, 1H), 1.88-2.05 (m, 2H), 1.17-1.85 (m, 16H), 0.50-68 (m, 1H), 0.28-0.42 (m, 2H), 0.02-0.12 (m, 2H). |
| 60 | 635 [M + Na]⁺ | (400 MHz, 60° C, DMSO-d₆) δ 11.22 (br, 1H), 9.41 (br, 1H), 8.10-8.20 (m, 1H), 7.40-7.55 (m, 1H), 6.90-7.10 (m, 1H), 5.15- 5.40 (m, 2H), 4.75-4.90 (m, 1H), 3.90-4.10 (m, 2H), 3.50-3.80 (m, 2H), 2.65-2.85 (m, 1H), 2.55-2.64 (m, 1H), 2.08-2.20 (m, 1H), 1.60-1.76 (m, 3H), 1.42-1.59 (m, 4H), 1.26-1.41 (m, 1H), 0.95-1.10 (m, 1H), 0.76-0.91 (m, 9H). |
| 61 | 591 [M + Na]⁺ | (400 MHz, 80° C, DMSO-d₆) δ 11.37 (br, 1H), 7.90-8.10 (m, 1H), 7.65-7.90 (m, 1H), 7.12-7.25 (m, 1H), 6.91-7.10 (m, 1H), 5.21-5.48 (m, 1H), 4.79-5.00 (m, 2H), 4.20-4.39 (m, 2H), 3.50- 3.70 (m, 2H), 2.72-2.85 (m, 1H), 2.60-2.70 (m, 1H), 2.09-2.20 (m, 1H), 1.71-1.85 (m, 1H), 1.32-1.61 (m, 7H), 1.20-1.31 (m, 3H), 1.05-1.19 (m, 2H), 0.75-0.90 (m, 9H). |
| 62 | 558 [M + Na]⁺ | (400 MHz, 80° C, DMSO-d₆) δ 11.49 (s, 1H), 7.50-7.82 (m, 1H), 7.20-7.35 (m, 1H), 7.02-7.19 (m, 1H), 6.81-7.01 (m, 2H), 4.80-5.30 (m, 3H), 3.90-4.20 (m, 2H), 3.55-3.80 (m, 2H), 2.61- 2.70 (m, 1H), 2.30-2.60 (m, 1H), 1.70-1.90 (m, 3H), 1.40-1.69 (m, 6H), 1.21-1.39 (m, 3H), 1.00-1.20 (m, 2H), 0.56-0.78 (m, 1H), 0.26-0.50 (m, 2H), 0.02-0.20 (m, 2H). |
| 63 | 556 [M + H]⁺ | (400 MHz, 80° C, DMSO-d₆) δ 11.02 (s, 1H), 7.65-7.80 (m, 1H), 7.10-7.22 (m, 1H), 6.85-7.03 (m, 2H), 5.10-5.35 (m, 2H), 4.70-5.00 (m, 1H), 3.85-4.10 (m, 2H), 3.60-3.80 (m, 1H), 3.50- 3.59 (m, 1H), 2.63-2.78 (m, 1H), 2.52-2.62 (m, 1H), 1.40-1.90 (m, 10H), 1.02-1.39 (m, 5H), 0.88-1.00 (m, 3H), 0.76-0.87 (m, 3H). |
| 64 | 595 [M + Na]⁺. | (400 MHz, DMSO-d₆) δ 11.90 (br, 1H), 8.10-8.25 (m, 1H), 7.90-8.09 (m, 1H), 7.20-7.39 (m, 1H), 5.12-5.36 (m, 1H), 4.90- 5.00 (m, 1H), 4.78-4.89 (m, 1H), 4.22-4.45 (m, 1H), 4.10-4.21 (m, 1H), 3.55-3.72 (m, 1H), 3.42-3.50 (m, 1H), 2.78-2.89 (m, 1H), 2.61-2.70 (m, 1H), 0.98-1.89 (m, 15H), 0.60-0.97 (m, 6H). |
| 65 | 574 [M + Na]⁺ | (400 MHz, 80° C, DMSO-d₆) δ 10.53 (br, 1H), 7.52-7.88 (m, 1H), 7.19-7.38 (m, 1H), 7.04-7.18 (m, 1H), 6.80-7.03 (m, 2H), 5.31-5.52 (m, 1H), 5.04-5.30 (m, 1H), 4.70-5.03 (m, 1H), 3.84- 4.09 (m, 2H), 3.51-3.62 (m, 2H), 2.60-2.71 (m, 1H), 2.52-2.59 (m, 1H), 2.08-2.11 (m, 1H), 1.71-1.81 (m, 1H), 1.42-1.70 (m, 6H), 1.22-1.38 (m, 3H), 1.04-1.21 (m, 3H), 0.75-0.97 (m, 9H). |
| 66 | 591 [M + Na]⁺ | (400 MHz, 80° C, DMSO-d₆) δ 11.43 (br, 1H), 7.92-8.10 (m, 1H), 7.35-7.60 (m, 1H), 7.20-7.34 (m, 1H), 6.89-7.12 (m, 1H), 5.20-5.40 (m, 1H), 4.80-5.11 (m, 2H), 4.10-4.40 (m, 2H), 3.50- 3.75 (m, 2H), 2.78-2.89 (m, 1H), 2.63-2.76 (m, 1H), 1.20-1.98 (m, 18H), 0.56-0.70 (m, 1H), 0.30-0.40 (m, 2H), 0.05-0.12 (m, 2H) |
| 67 | 615 [M + Na]⁺ | (400 MHz, 80° C., DMSO-d₆) δ 11.45 (br, 1H), 9.00-9.25 (m, 1H), 7.95-8.10 (m, 1H), 7.20-7.35 (m, 1H), 6.95-7.11 (m, 1H), 5.20-5.40 (m, 1H), 4.80-5.05 (m, 2H), 4.15-4.35 (m, 2H), 3.55- 3.79 (m, 2H), 2.75-2.90 (m, 1H), 2.60-2.70 (m, 1H), 1.80-2.00 (m, 3H), 1.58-1.79 (m, 4H), 1.41-1.57 (m, 4H), 1.20-1.40 (m, 1H), 0.55-0.70 (m, 1H), 0.25-0.45 (m, 2H), 0.05-0.19 (m, 2H) |
| 68 | 617 [M + Na]⁺ | (400 MHz, 80° C., DMSO-d₆) δ 11.48 (br, 1H), 9.19 (s, 1H), 7.90-8.06 (m, 1H), 7.18-7.28 (m, 1H), 6.92-7.10 (m, 1H), 5.28- 5.40 (m, 1H), 4.82-5.00 (m, 2H), 4.25-4.40 (m, 1H), 4.08-4.20 (m, 1H), 3.52-3.75 (m, 2H), 2.70-2.85 (m, 1H), 2.54-2.69 (m, 1H), 1.76-2.00 (m, 3H), 1.51-1.71 (m, 4H), 1.32-1.50 (m, 5H), 1.15-1.31 (m, 1H), 0.85-0.92 (m, 3H), 0.73-0.84 (m, 3H). |
| 69 | 601 [M + Na]⁺ | (400 MHz, 80° C, DMSO-d₆) δ 11.45 (br, 1H), 9.34 (br, 1H), 7.90-8.10 (m, 1H), 7.18-7.35 (m, 1H), 6.90-7.17 (m, 1H), 5.28- 5.45 (m, 1H), 4.80-5.10 (m, 2H), 4.25-4.50 (m, 1H), 4.00-4.24 (m, 1H), 3.55-3.80 (m, 2H), 2.72-2.88 (m, 1H), 2.58-2.71 (m, 1H), 1.75-2.00 (m, 3H), 1.54-1.74 (m, 4H), 1.35-1.53 (m, 5H), 1.19-1.34 (m, 1H), 0.75-0.99 (m, 6H) |
| 70 | 341 (fragment peak) | (400 MHz, 80° C, DMSO-d₆) δ 11.48 (br, 1H), 7.90-8.00 (m, 1H), 7.40-7.60 (m, 1H), 7.18-7.38 (m, 1H), 6.95-7.10 (m, 1H), 5.25-5.45 (m, 1H), 4.80-5.00 (m, 2H), 4.22-4.41 (m, 1H), 4.10- 4.21 (m, 1H), 3.50-3.70 (m, 2H), 2.72-2.86 (m, 1H), 2.53-2.70 (m, 1H), 1.70-2.00 (m, 3H), 1.50-1.68 (m, 4H), 1.32-1.49 (m, 11H), 1.11-1.31 (m, 1H), 0.76-0.95 (m, 6H) |
| 71 | 619 [M + Na]⁺ | (400 MHz, 80° C, DMSO-d₆) δ 11.60 (br, 1H), 9.37 (s, 1H), 7.98-8.10 (m, 1H), 7.18-7.32 (m, 1H), 5.25-5.40 (m, 1H), 4.85- 5.05 (m, 2H), 4.21-4.40 (m, 1H), 4.10-4.20 (m, 1H), 3.50-3.75 (m, 2H), 2.72-2.90 (m, 1H), 2.58-2.69 (m, 1H), 1.70-1.99 (m, 3H), 1.51-1.69 (m, 4H), 1.31-1.50 (m, 5H), 1.10-1.30 (m, 1H), 0.82-0.95 (m, 3H), 0.75-0.81 (m, 3H) |
| 72 | 561 [M + Na]⁺ | (400 MHz, 80° C., DMSO-d₆) δ 10.95 (br, 1H), 8.08-8.20 (m, 1H), 7.35-7.55 (m, 2H), 6.90-7.00 (m, 1H), 5.00-5.30 (m, 2H), 4.70-4.88 (m, 1H), 4.09-4.20 (m, 1H), 3.92-4.08 (m, 1H), 3.61- 3.80 (m, 1H), 3.45-3.60 (m, 1H), 2.65-2.79 (m, 1H), 2.53-2.63 (m, 1H), 1.20-1.90 (m, 16H), 0.60-0.74 (m, 1H), 0.30-0.50 (m, 2H), 0.05-0.20 (m, 2H) |
| 73 | 565 [M + Na]⁺ | (400 MHz, DMSO-d₆) δ 11.12 (br, 1H), 8.98 (br, 1H), 8.05- 8.15 (m, 1H), 7.40-7.50 (m, 1H), 6.90-7.00 (m, 1H), 5.10-5.35 (m, 2H), 4.75-4.85 (m, 1H), 3.90-4.10 (m, 2H), 3.60-3.75 (m, 1H), 3.48-3.59 (m, 1H), 2.61-2.75 (m, 1H), 2.50-2.59 (m, 1H), 1.40-1.80 (m, 10H), 1.10-1.30 (m, 1H), 0.88-0.93 (m, 3H), 0.75-0.85 (m, 3H) |
| 74 | 327 (fragment peak) | (400 MHz, 80° C., DMSO-d₆) δ 11.12 (br, 1H), 8.05-8.20 (m, 1H), 7.40-7.50 (m, 2H), 6.85-7.00 (m, 1H), 5.10-5.27 (m, 2H), 4.70-4.84 (m, 1H), 3.90-4.08 (m, 2H), 3.60-3.72 (m, 1H), 3.45- 3.59 (m, 1H), 2.62-2.75 (m, 1H), 2.52-2.61 (m, 1H), 1.65-1.85 (m, 2H),1.58-1.64 (m, 3H), 1.42-1.57 (m, 6H), 1.31-1.41 (m, 5H), 1.10-1.29 (m, 1H), 0.86-0.95 (m, 3H), 0.76-0.85 (m, 3H) |
| 75 | 323 (fragment peak) | (400 MHz, 80º C, DMSO-d₆) δ 11.11 (br, 1H), 8.05-8.20 (m, 1H), 7.60-7.80 (m, 1H), 7.40-7.59 (m, 1H), 6.90-7.10 (m, 1H), 4.99-5.32 (m, 2H), 4.80-4.98 (m, 1H), 4.10-4.27 (m, 1H), 3.90- 4.09 (m, 1H), 3.61-3.75 (m, 1H), 3.47-3.60 (m, 1H), 2.65-2.80 (m, 1H), 2.55-2.64 (m, 1H), 1.79-1.90 (m, 2H), 1.70-1.78 (m, 1H), 1.46-1.69 (m, 6H), 1.32-1.45 (m, 1H), 1.22-1.31 (m, 2H), 1.03-1.21 (m, 2H), 0.60-0.78 (m, 1H), 0.30-0.50 (m, 2H), 0.05- 0.20 (m, 2H) |
| 76 | 325 (fragment peak) | (400 MHz, 80° C., DMSO-d₆) δ 11.11 (br, 1H), 8.05-8.15 (m, 1H), 7.68-7.80 (m, 1H), 7.40-7.50 (m, 1H), 6.90-7.00 (m, 1H), 5.10-5.25 (m, 2H), 4.76-4.92 (m, 1H), 4.00-4.09 (m, 1H), 3.88- 3.99 (m, 1H), 3.60-3.75 (m, 1H), 3.45-3.59 (m, 1H), 2.60-2.78 (m, 1H), 2.51-2.59 (m, 1H), 1.63-1.84 (m, 3H), 1.40-1.62 (m, 7H), 1.18-1.30 (m, 3H), 1.00-1.17 (m, 2H), 0.85-0.95 (m, 3H), 0.75-0.84 (m, 3H) |
| 77 | 599 [M + H]⁺ | (400 MHz, 80° C, DMSO-d₆) δ 11.11 (br, 1H), 9.25 (br, 1H), 8.08-8.18 (m, 1H), 7.35-7.45 (m, 1H), 6.88-7.00 (m, 1H), 5.10- 5.30 (m, 2H), 4.80-4.90 (m, 1H), 3.90-4.05 (m, 2H), 3.62-3.75 (m, 1H), 3.49-3.61 (m, 1H), 2.62-2.72 (m, 1H), 2.52-2.61 (m, 1H), 1.40-1.80 (m, 10H), 1.10-1.25 (m, 1H), 0.85-0.93 (m, 3H), 0.76-0.84 (m, 3H) |
| 78 | 625 [M + Na]⁺ | (400 MHz, 80° C, DMSO-d₆) δ 11.35 (br, 1H), 7.90-8.05 (m, 1H), 7.30-7.44 (m, 1H), 7.15-7.28 (m, 1H), 6.95-7.05 (m, 1H), 5.16-5.29 (m, 1H), 4.88-5.00 (m, 1H), 4.79-4.87 (m, 1H), 4.15- 4.28 (m, 2H), 3.56-3.72 (m, 1H), 3.44-3.55 (m, 1H), 2.75-2.85 (m, 1H), 2.63-2.74 (m, 1H), 1.70-1.85 (m, 1H), 1.40-1.69 (m, 9H), 1.21-1.30 (m, 2H), 1.10-1.20 (m, 2H), 0.50-0.70 (m, 1H), 0.27-0.41 (m, 2H), 0.00-0.12 (m, 2H) |
| 79 | 375 (fragment peak) | (400 MHz, 80° C, DMSO-d₆) δ 7.95-8.05 (m, 1H), 7.32-7.50 (m, 1H), 7.15-7.30 (m, 1H), 6.97-7.10 (m, 1H), 5.19-5.35 (m, 1H), 4.86-5.00 (m, 1H), 4.70-4.85 (m, 1H), 4.12-4.35 (m, 2H), 3.55-3.71 (m, 1H), 3.40-3.54 (m, 1H), 2.71-2.86 (m, 1H), 2.62- 2.70 (m, 1H), 1.69-1.85 (m, 1H), 1.35-1.67 (m, 10H), 1.22-1.34 (m, 2H), 1.10-1.21 (m, 2H), 0.83-0.95 (m, 3H), 0.70-0.82 (m, 3H) |
| 80 | 311 (fragment peak) | (400 MHz, 80º C, DMSO-d₆) δ 11.45 (s, 1H), 7.90-8.10 (m, 1H), 7.30-7.50 (m, 1H), 7.15-7.29 (m, 1H), 6.90-7.08 (m, 1H), 5.30-5.47 (m, 1H), 4.86-5.00 (m, 1H), 4.70-4.85 (m, 1H), 4.15- 4.35 (m, 2H), 3.54-3.72 (m, 1H), 3.40-3.53 (m, 1H), 2.75-2.90 (m, 1H), 2.60-2.74 (m, 1H), 1.82-1.95 (m, 1H), 1.70-1.81 (m, 1H), 1.50-1.60 (m, 6H), 1.46-1.50 (m, 3H), 1.39-1.45 (m, 4H), 1.34-1.38 (m, 1H), 0.90-1.05 (m, 3H), 0.22-0.35 (m, 2H), 0.10- 0.21 (m, 2H) |
| 81 | 341 (fragment peak) | (400 MHz, 80º C, DMSO-d₆) δ 11.50 (br, 1H), 9.22 (br, 1H), 7.90-8.05 (m, 1H), 7.15-7.30 (m, 1H), 6.95-7.10 (m, 1H), 5.35- 5.50 (m, 1H), 4.70-5.02 (m, 2H), 4.15-4.40 (m, 2H), 3.58-3.73 (m, 1H), 3.43-3.57 (m, 1H), 2.71-2.90 (m, 1H), 2.60-2.70 (m, 1H), 1.82-1.98 (m, 1H), 1.38-1.80 (m, 9H), 0.88-1.02 (m, 3H), 0.21-0.35 (m, 2H), 0.10-0.20 (m, 2H) |

### Example A

### SARS-Cov-2 3CLpro

Protease assays were performed in 384-well low volume polypropylene microtiter plates at ambient temperature. For the duplex assay, 3CLpro was added using a Multidrop Combi (Thermo Scientific; Waltham, MA) and preincubated for 30 mins with small molecules. The reactions were initiated by the addition of the two peptide substrates. The reactions were incubated for 30 mins and quenched by the addition of 0.5% formic acid (final) with subsequent neutralization using 1% sodium bicarbonate (final). Internal standard peptides were added in 20 mM Hepes pH 8.0 for quantitation of the protease products. For SAMDI-MS analysis, 2 μL of each reaction mixture was transferred using a 384-channel automated liquid handler to SAMDI biochip arrays functionalized with a neutravidin-presenting self-assembled monolayer. The SAMDI arrays were incubated for 1 h in a humidified chamber to allow the specific immobilization of the biotinylated peptide substrates, cleaved products and internal standards. The samples were purified by washing the SAMDI arrays with deionized ultrafiltered water and dried with compressed air. A matrix comprising alpha-cyano cinnamic acid in 80% acetonitrile:20% aqueous ammonium citrate was applied in an automated format by dispensing 50 nL to each spot in the array. SAMDI-MS was performed using reflector-positive mode on an AB Sciex TOF-TOF 5800 System (AB Sciex, Framingham, MA) with 400 shots/spot analyzed in a random raster sampling. For data analysis, area under the curves (peaks) (AUCs) for the product and internal standard were calculated using the TOF/TOF Series Explorer (AB Sciex), and the amount of product formed was calculated using the equation (AUC product/AUC internal standard). The amount of product generated was calculated using the ratio of product area under the curve (AUC) divided by the AUC of the internal standard. Negative controls were pre-quenched with 0.5% formic acid final. Assay robustness was determined by Z-Factor. The IC₅₀s were determined by fitting the curves using a four-parameter equation in Graphpad Prism 8.

Table A indicates related IC₅₀ values for the tested compounds where ‘A’ indicates an IC₅₀<20 nM, ‘B’ indicates an IC₅₀ of ≥20 nM and <200 nM, ‘C’ indicates an IC₅₀≥200 nM and <2000 nM, ‘D’ indicates an IC₅₀>2000 nM. As shown by the data in Table A, compounds described herein (including pharmaceutically acceptable salts thereof) can effectively inhibit and be used to treat a coronavirus and rhinovirus.

TABLE A

| Compound | SARS-Cov-2 |
|---|---|
| 1B | A |
| 2B | A |
| 3A | B |
| 3B | A |
| 4A | D |
| 4B | A |
| 5 | A |
| 6 | A |
| 7 | A |
| 8 | A |
| 9 | A |
| 10 | A |
| 11 | A |
| 13 | A |
| 14 | A |
| 15 | A |
| 16 | A |
| 17 | A |
| 18 | A |
| 19 | A |
| 20 | A |
| 21 | A |
| 22 | A |
| 23A | D |
| 23B | A |
| 24 | C |
| 25 | C |
| 25-1 | B |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |

### Example B

### Coronavirus Assay

OC43 Coronavirus Assay in HeLa cells

The human beta-coronavirus OC43 was purchased from ATCC (Manassas, VA) and propagated using HCT-8 human colorectal epithelial cells (ATCC). HeLa human cervical epithelial cells (ATCC) were used as susceptible host cell lines and were cultured using EMEM media, supplemented with 10% fetal bovine serum (FBS), 1% (v/v) penicillin/streptomycin (P/S), 1% (v/v) HEPES and 1% (v/v) Cellgro Glutagro™ supplement (all Corning, Manassas, VA) at 37° C. For the OC43 antiviral assay, 1.5×10⁴ HeLa cells per well were plated in 100 μL complete media in white 96-well plates with clear bottoms at 37° C. for up to 24 h to facilitate attachment and allow cells to recover from seeding stresses. Next day, the cell culture medium was removed. Serially diluted compounds in 100 μL assay media (EMEM, 2% FBS, 1% P/S, 1% Cellgro Glutagro™ supplement, 1% HEPES) were added to the cells and incubated for 4 h at 37° C. in a humidified 5% CO₂ incubator. 100 μL of OC43 virus stock was diluted to a concentration known to produce optimal cytopathic effect, inducing 80-90% reduction in cell viability. 96-well plates were incubated for 6 days at 33° C.; each plate contains uninfected control wells as well as virus-infected wells that were not treated with compound. Cytotoxicity plates without the addition of OC43 virus were carried out in parallel. At the end of the incubation period, 100 μL cell culture supernatant was replaced with 100 μL cell-titer-glo reagent (Promega, Madison, WI) and incubated for at least 10 min at rt prior to measuring luminescence. Luminescence was measured on a Perkin Elmer (Waltham, MA) Envision plate reader. Antiviral % inhibition was calculated as follows: [(Compound treated cells infected sample)−(no compound infected control)]/[(Uninfected control)−(no compound infected control)]*100; Using GraphPad (San Diego, CA) prism software version 8.3.1, the antiviral dose-response plot was generated as a sigmoidal fit, log(inhibitor) vs response-variable slope (four parameters) model and the EC₅₀ was calculated which is the predicted compound concentration corresponding to a 50% inhibition of the viral cytopathic effect.

Table B indicates related EC₅₀ and CC₅₀ values for the tested compounds ‘A’ indicates an EC₅₀<100 nM, ‘B’ indicates an EC₅₀ of ≥100 nM and <1000 nM, ‘C’ indicates an EC₅₀≥1000 nM and <10000 nM and ‘D’ indicates an EC₅₀≥10000 nM and <100000 nM. For CC₅₀, the values are reported in micromolar (μm), ‘A’ indicates a CC₅₀≥10000 nM and ‘B’ indicates a CC₅₀≥1 μM and <10 μM.

TABLE B

| Compound | EC₅₀ | CC₅₀ |
|---|---|---|
| 1A | D | A |
| 1B | A | A |
| 2A | D | A |
| 2B | A | A |
| 3A | C | A |
| 3B | A | A |
| 4B | A | A |
| 5 | A | A |
| 6 | A | A |
| 7 | A | A |
| 8 | B | A |
| 9 | A | A |
| 10 | B | A |
| 11 | A | A |
| 12 | B | A |
| 13 | B | A |
| 14 | B | A |
| 15 | A | A |
| 16 | A | A |
| 17 | A | A |
| 18 | B | A |
| 19 | A | A |
| 20 | B | A |
| 21 | A | A |
| 22 | A | A |
| 23 | C | A |
| 24 | C | A |
| 25-1 | A | A |
| 25 | C | A |
| 26 | B | A |
| 27 | A | A |
| 28 | A | A |
| 29 | A | A |
| 30 | A | A |
| 31 | A | A |
| 32 | A | A |
| 33 | B | A |
| 34 | A | A |
| 35 | A | A |
| 36 | A | A |
| 37 | A | A |
| 38 | A | A |
| 39 | A | A |
| 40 | A | A |
| 41 | A | A |
| 42 | A | A |
| 43 | B | A |
| 44 | B | A |
| 45 | A | A |
| 46 | A | A |
| 47 | A | A |
| 48 | A | A |
| 49 | A | A |
| 50 | A | A |
| 51 | A | A |
| 52 | A | A |
| 53 | B | A |
| 54 | A | A |
| 55 | B | A |
| 56 | B | A |
| 57 | A | A |
| 58 | A | A |
| 59 | B | A |
| 60 | A | A |
| 61 | B | A |
| 62 | A | A |
| 63 | A | A |
| 64 | B | A |
| 65 | A | A |
| 66 | B | A |
| 67 | A | A |
| 68 | B | A |
| 69 | B | A |
| 70 | B | A |
| 71 | B | A |
| 72 | A | A |
| 73 | A | A |
| 74 | B | A |
| 75 | B | A |
| 76 | B | A |
| 77 | A | A |
| 78 | A | A |

Tables A and B demonstrate that compounds described herein (including pharmaceutically acceptable salts thereof) can effectively inhibit and treat a coronavirus.

### Example C

### Picornavirus & Norovirus Assays

Compounds of Formula (I), including pharmaceutically acceptable salts thereof, are tested following a protocol similar to the protocol described in one of the following articles: Kim et al., Journal of Virology (2012) 86(21):11754-11762, Zhang et al, JACS (2020) (https://dx.doi.org/10.1021/acs.jmedchem.9b01828), and U.S. Pat. No. 9,603,864.

The protocols of Kim et al., and Zhang et al., can be used to test for activity against a picornavirus and norovirus.

### Example D

For the cathepsin L assay, 10 pM of human cathepsin L (R&D Systems; Minneapolis, MN) was preincubated for 30 mins with test compounds. Reactions were initiated by the addition of a peptide substrate Z—FR-AMC (final concentration 2 μM, Anaspec; Fremont, CA). Fluorescence was measured at 2-minute intervals for 30 mins using a 355/460 excitation/emission filter module on an Envision plate reader (Perkin Elmer; Waltham, MA). The IC₅₀ values were calculated for each assay by fitting the curves using a four-parameter equation in GraphPad Prism. Compound 23B has an IC₅₀ value of ≥10,000. Thus, compounds of Formula (I) are selective for a coronavirus protease compared to a host protease, such as cathepsin L.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

1. A compound of Formula (I), or a pharmaceutically acceptable salt thereof, having the structure: 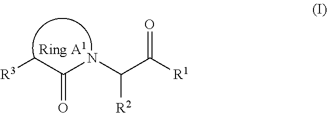
wherein:
R¹ is selected from the group consisting of 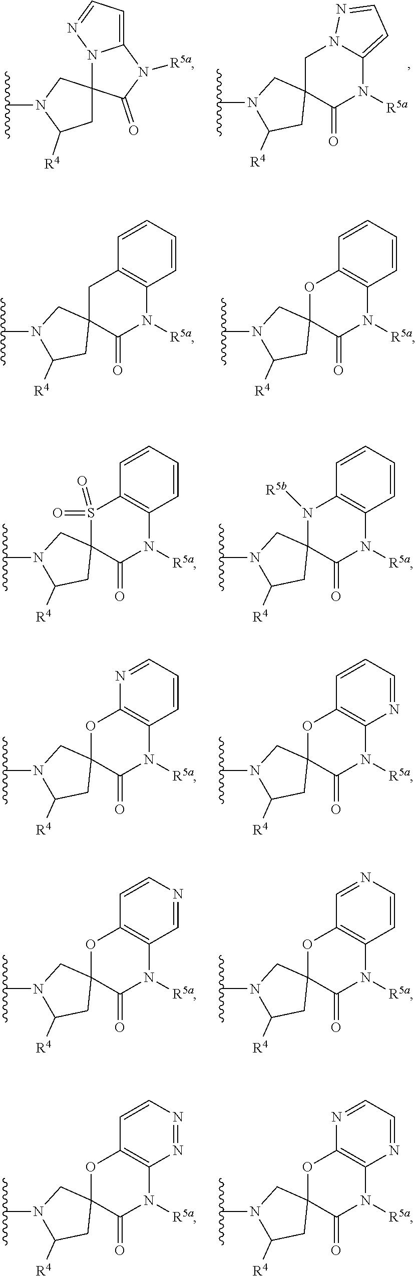 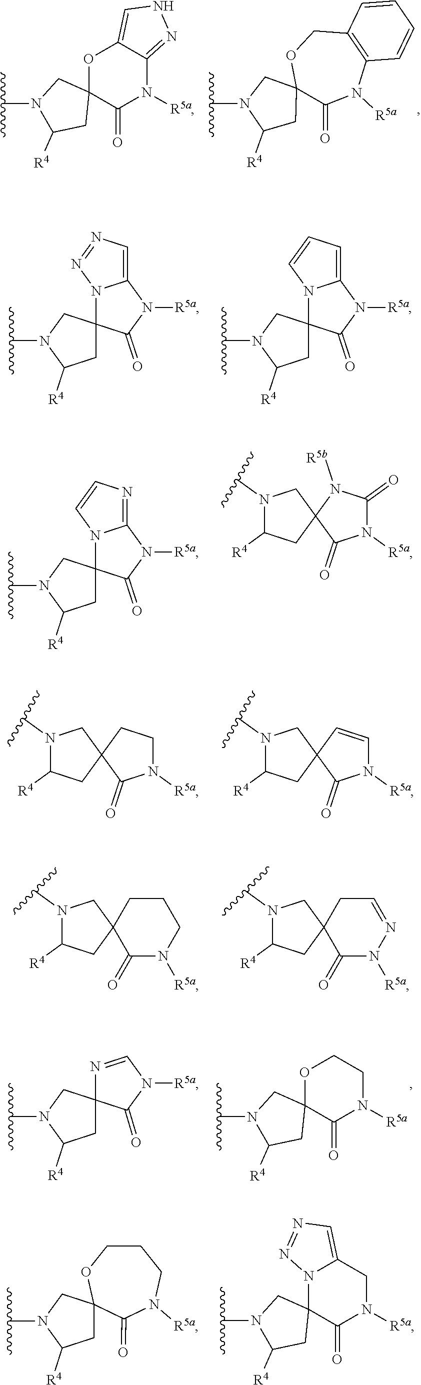 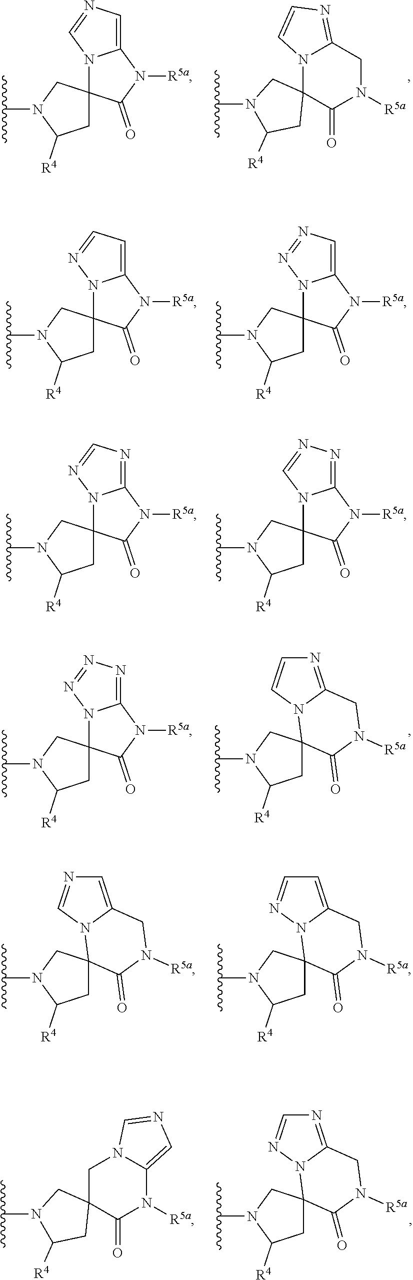 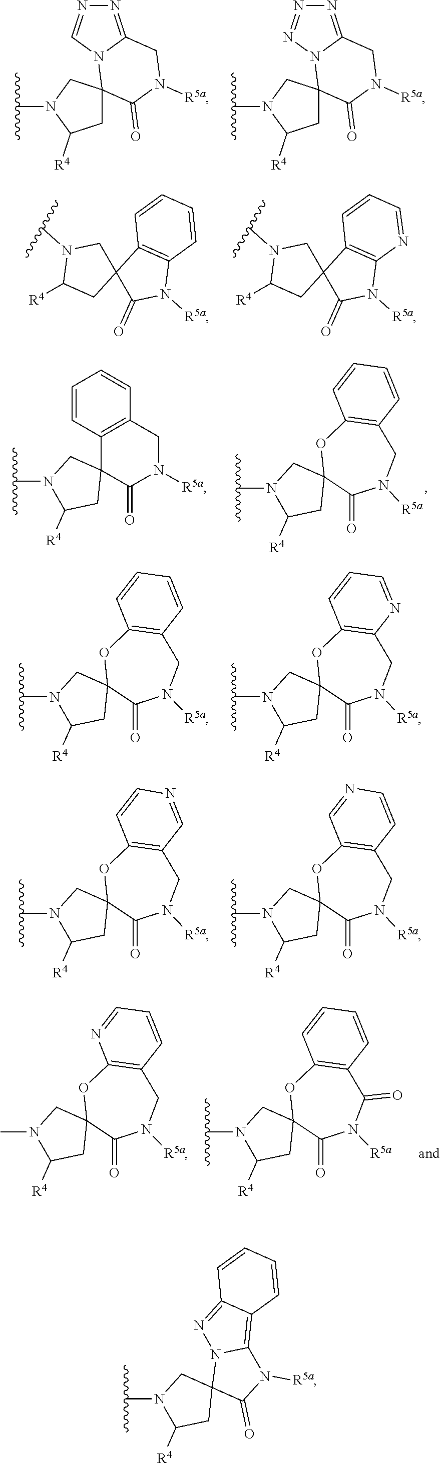
wherein each is optionally substituted with one or more moieties independently selected from the group consisting of halogen, cyano, hydroxy, an unsubstituted C₁₋₆ alkyl, an unsubstituted —O(C₁₋₆ alkyl), an unsubstituted C₁₋₄ haloalkyl, an unsubstituted C₁₋₄ haloalkoxy, an unsubstituted or a substituted phenoxy, an unsubstituted or a substituted C₃₋₆ cycloalkyl, an unsubstituted or a substituted phenyl, an unsubstituted or a substituted benzyl, an unsubstituted or a substituted 5- or 6-membered heteroaryl, —S(═O)₂ (an unsubstituted C₁₋₄ alkyl), —NR^{N1}R^{N2} and —C(═O)—NR^{N1}R^{N2}, wherein R^{N1} and R^{N2} are independently hydrogen or an unsubstituted C₁₋₄ alkyl, or R^{N1} and R^{N2} are taken together to form a monocyclic heterocyclyl;
R² is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₁₀ cycloalkyl, an unsubstituted or a substituted C₃₋₁₀ cycloalkenyl, an unsubstituted or a substituted aryl, an unsubstituted or a substituted aryl(alkyl), an unsubstituted or a substituted heteroaryl, an unsubstituted or a substituted heteroaryl(alkyl), an unsubstituted or a substituted heterocyclyl or an unsubstituted or a substituted heterocyclyl(alkyl);
R³ is 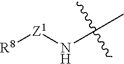
or R⁹;
Ring A¹ is a 5-, 6-, 7-, 8- or 9-membered heterocyclyl that optionally includes a double bond and optionally is fused to an unsubstituted or a substituted monocyclic C₃₋₄ cycloalkyl;
Z¹ is —C(═O)— or —S(═O)₂—;
R⁴ is selected from the group consisting of cyano, an unsubstituted or a substituted C₂₋₅ alkynyl, an unsubstituted or a substituted acyl, an unsubstituted or a substituted ketoamide, —C(═O)NH₂, —CH(OH)—(S(═O)₂—OH), —CH(OH)—(S(═O)₂—O—), —CH(OH)((P=O)(OR⁶)₂) and —C(═O)CH₂—O—((P=O)(OR⁷)₂);
R^{5a} is selected from the group consisting of hydrogen, an unsubstituted or a substituted C₁₋₄ alkyl, an unsubstituted or a substituted C₂₋₄ alkenyl and an unsubstituted or a substituted C₃₋₆ cycloalkyl;
R^{5b} is selected from the group consisting of hydrogen, an unsubstituted or a substituted C₁₋₄ alkyl, an unsubstituted or a substituted C₂₋₄ alkenyl and an unsubstituted or a substituted C₃₋₆ cycloalkyl;
each R⁶ and each R⁷ are independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl);
R⁸ is selected from the group consisting of an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted phenyl, an unsubstituted or a substituted monocyclic heteroaryl, an unsubstituted or a substituted monocyclic heterocyclyl, an unsubstituted or a substituted alkoxy and —NR¹⁴R¹⁵, wherein the substituted C₁₋₆ alkyl is substituted 1 or 2 times with a substituent selected from the group consisting of hydroxy and an unsubstituted C₁₋₄ alkoxy, wherein the substituted monocyclic C₃₋₆ cycloalkyl is substituted 1, 2, 3 or 4 times with a substituent independently selected from the group consisting of halogen, an unsubstituted C₁₋₄ alkyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl is substituted 1 or 2 times with a substituent independently selected from the group consisting of an unsubstituted C₁₋₄ alkoxy and an unsubstituted or a substituted C₃₋₆ cycloalkyl;
R⁹ is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 12-membered monocyclic heterocyclyl, an unsubstituted or a substituted 5- to 12-membered bicyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹⁰, —NR¹¹R¹² or —C(═O)—NR^{13A}R^{13B};
R¹⁰ is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl);
R¹¹ and R¹² are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl);
R^{13A} is hydrogen or an unsubstituted C₁₋₃ alkyl;
R^{13B} is an unsubstituted or a substituted aryl, an unsubstituted or a substituted heteroaryl or an unsubstituted or a substituted 3- to 8-membered monocyclic heterocyclyl;
R¹⁴ and R¹⁵ are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or
R¹⁴ and R¹⁵ are taken together along with the nitrogen to which they are connected to form an unsubstituted or a substituted 3-8 membered heterocyclyl.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. The compound of claim 1, wherein Ring A¹ is an 8-membered heterocyclyl that does not include a double bond and is not fused to an unsubstituted or a substituted monocyclic C₃₋₄ cycloalkyl or Ring A¹ is an 8-membered heterocyclyl that does include a double bond and is not fused to an unsubstituted or a substituted monocyclic C₃₋₄ cycloalkyl.

15. (canceled)

16. The compound of claim 1, wherein Ring A¹ is an 8-membered heterocyclyl that does not include a double bond and is fused to an unsubstituted or a substituted monocyclic C₃₋₄ cycloalkyl or Ring A¹ is an 8-membered heterocyclyl that does include a double bond and is fused to an unsubstituted or a substituted monocyclic C₃₋₄ cycloalkyl.

17. (canceled)

18. (canceled)

19. (canceled)

20. (canceled)

21. (canceled)

22. The compound of claim 1, wherein Ring A¹ is selected from the group consisting of

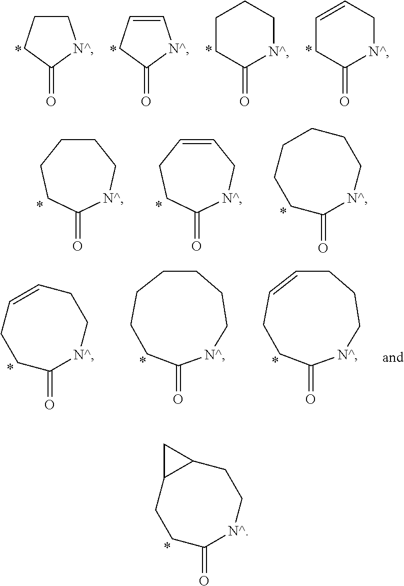

23. The compound of claim 1, wherein R¹ is selected from the group consisting of: 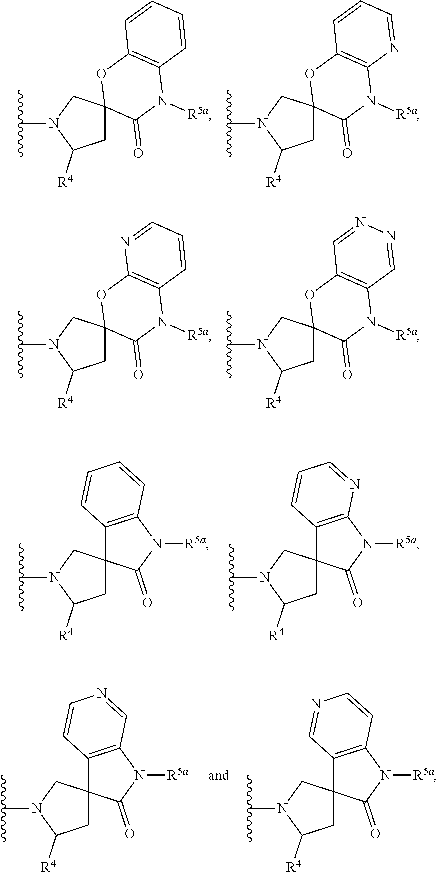
wherein each is unsubstituted or substituted.

24. The compound of claim 1, wherein R¹ is selected from the group consisting of: 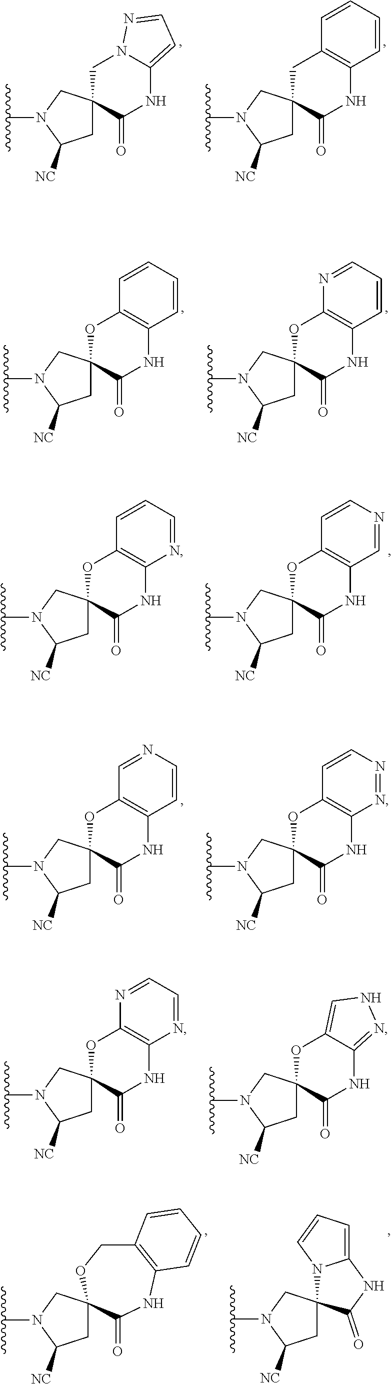 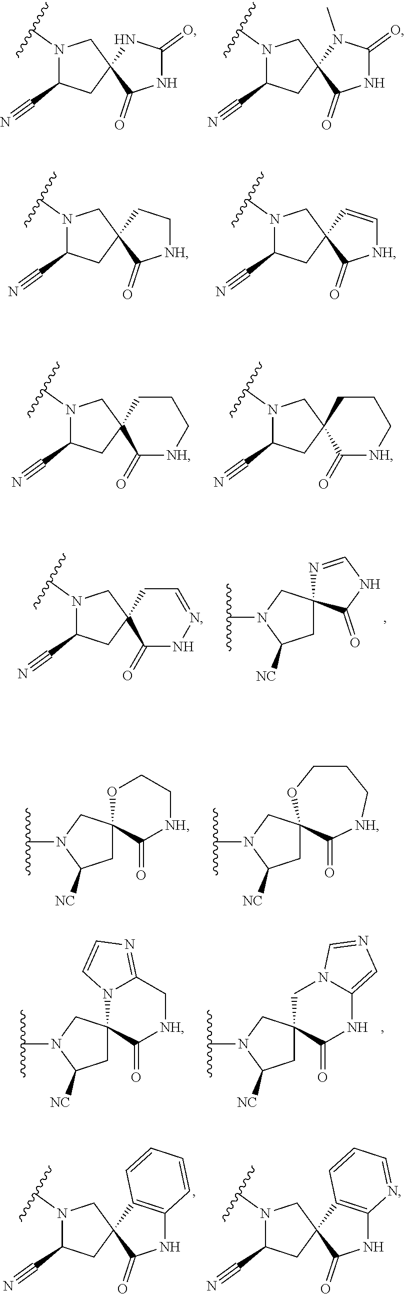 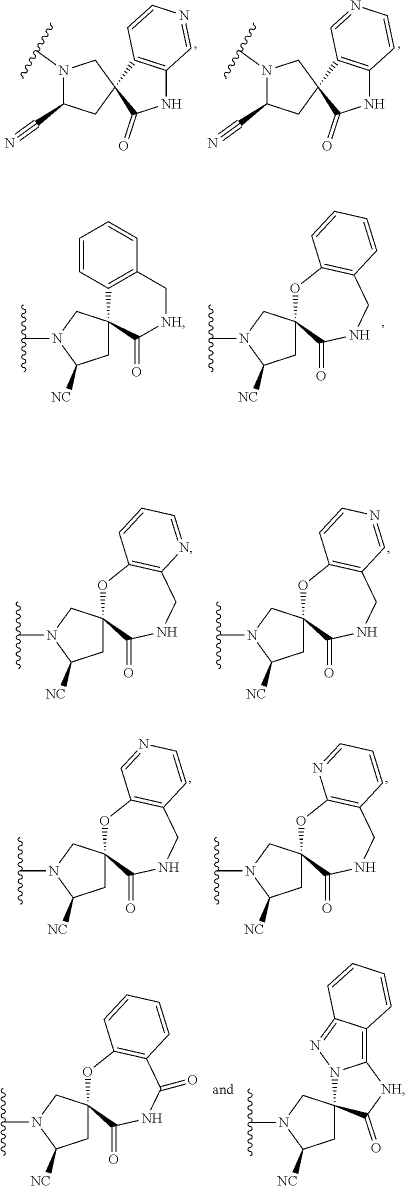
wherein each is unsubstituted or substituted.

25. (canceled)

26. The compound of claim 1, wherein R¹ is selected from the group consisting of:

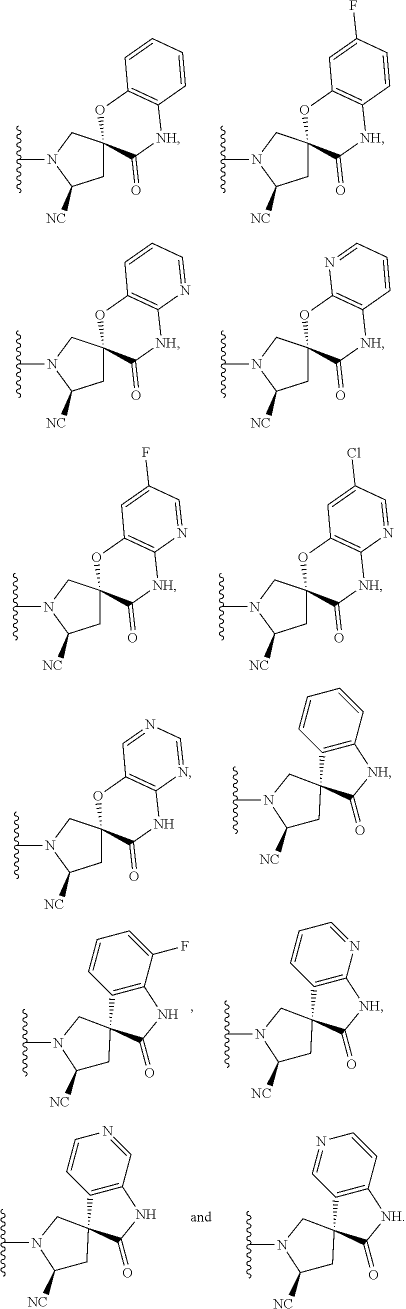

27. The compound of claim 1, wherein R² is an unsubstituted or a substituted C₁₋₈ alkyl.

28. (canceled)

29. The compound of claim 27, wherein R² is an unsubstituted C₁₋₄ alkyl.

30. (canceled)

31. The compound of claim 27, wherein R² is C₁₋₈ alkyl substituted with an unsubstituted or a substituted monocyclic C₃₋₆ cycloalkyl.

32. (canceled)

33. (canceled)

34. (canceled)

35. (canceled)

36. The compound of claim 1, wherein R² is selected from the group consisting of:

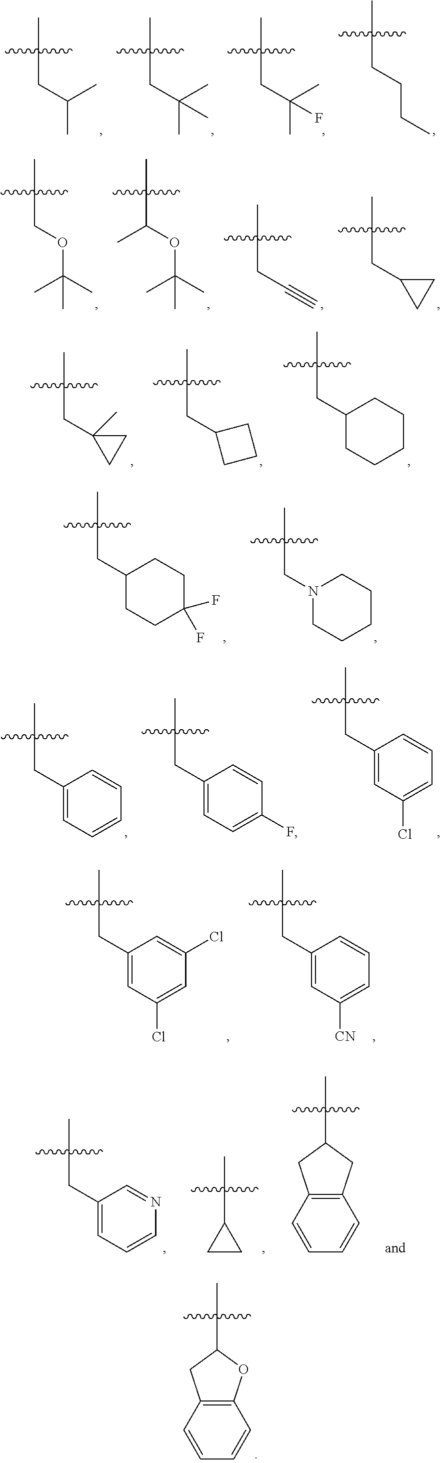

37. The compound of claim 36, wherein R² is

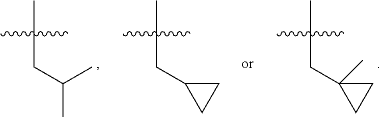

38. (canceled)

39. (canceled)

40. (canceled)

41. (canceled)

42. (canceled)

43. (canceled)

44. (canceled)

45. The compound of claim 1, wherein R⁴ is cyano.

46. (canceled)

47. The compound of claim 1, wherein R³ is 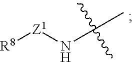
and Z¹ is —C(═O)—.

48. (canceled)

49. (canceled)

50. The compound of claim 47, wherein R⁸ is an unsubstituted C₁₋₆ haloalkyl.

51. (canceled)

52. The compound of claim 47, wherein R⁸ is an unsubstituted or substituted monocyclic C₃₋₆ cycloalkyl or an unsubstituted or a substituted alkoxy.

53. (canceled)

54. (canceled)

55. (canceled)

56. (canceled)

57. (canceled)

58. (canceled)

59. (canceled)

60. The compound of claim 1, wherein R³ is R⁹.

61. (canceled)

62. (canceled)

63. (canceled)

64. (canceled)

65. (canceled)

66. (canceled)

67. (canceled)

68. (canceled)

69. The compound of claim 60, wherein R⁹ is selected from the group consisting of: 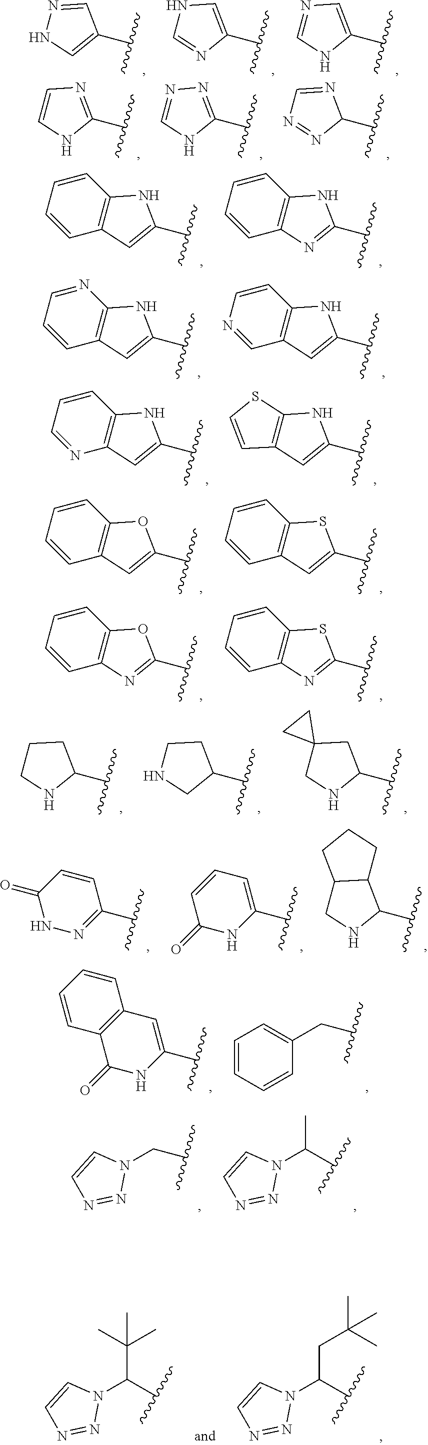
wherein each is unsubstituted or substituted.

70. (canceled)

71. (canceled)

72. (canceled)

73. (canceled)

74. (canceled)

75. (canceled)

76. (canceled)

77. (canceled)

78. (canceled)

79. (canceled)

80. The compound of claim 1, wherein R³ is selected from the group consisting of: 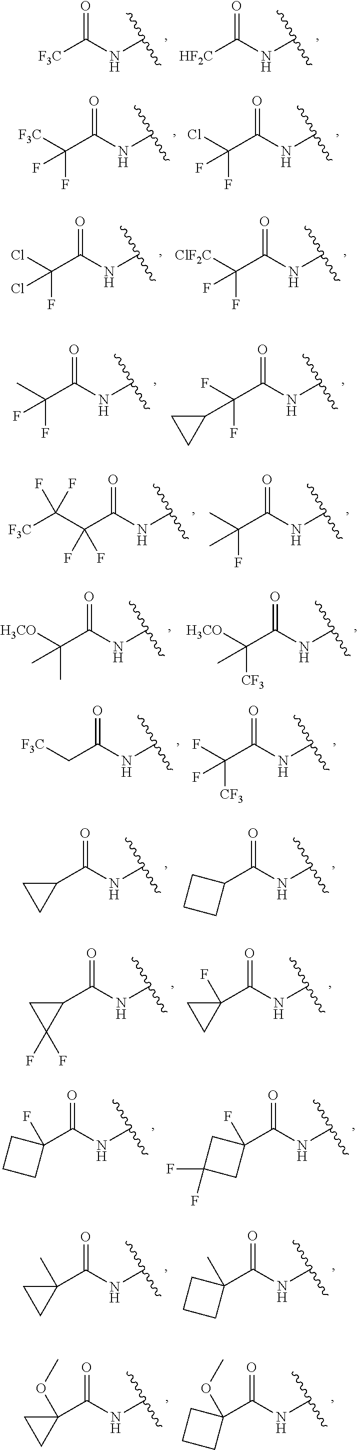 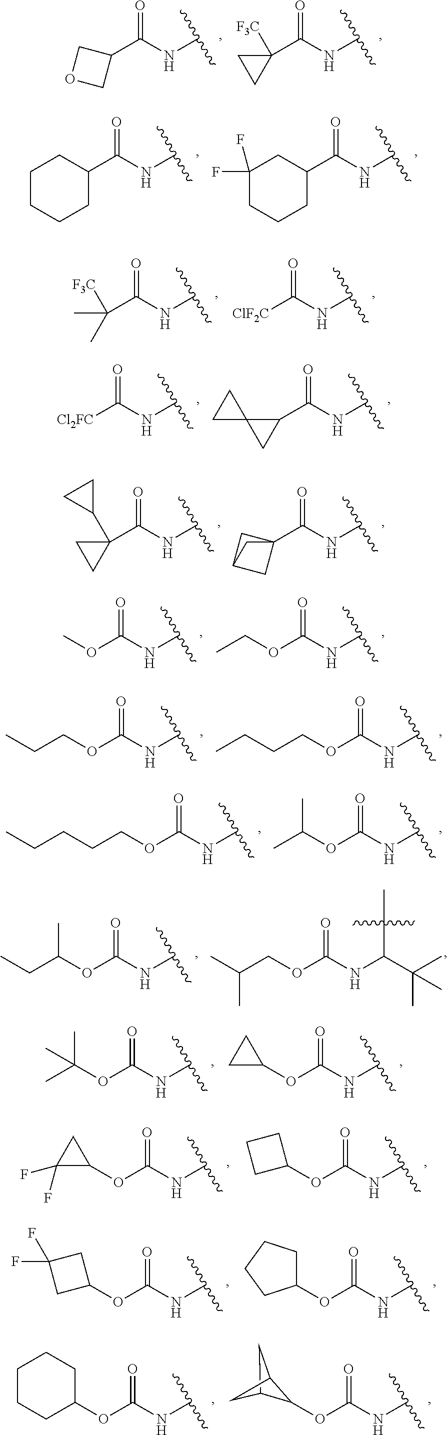 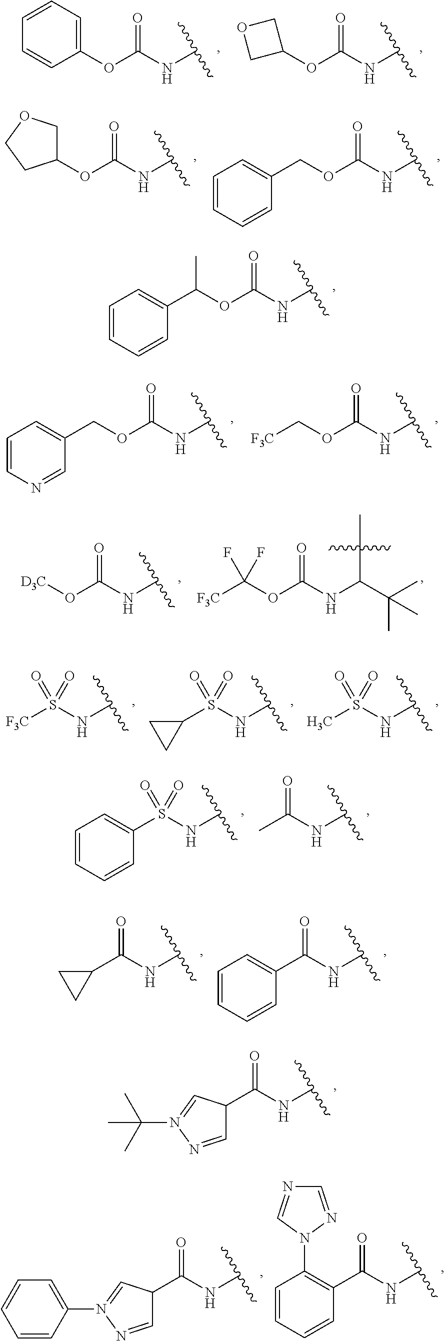 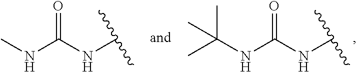
wherein each phenyl and can be substituted or unsubstituted.

81. The compound of claim 1, wherein R³ is selected from the group consisting of:

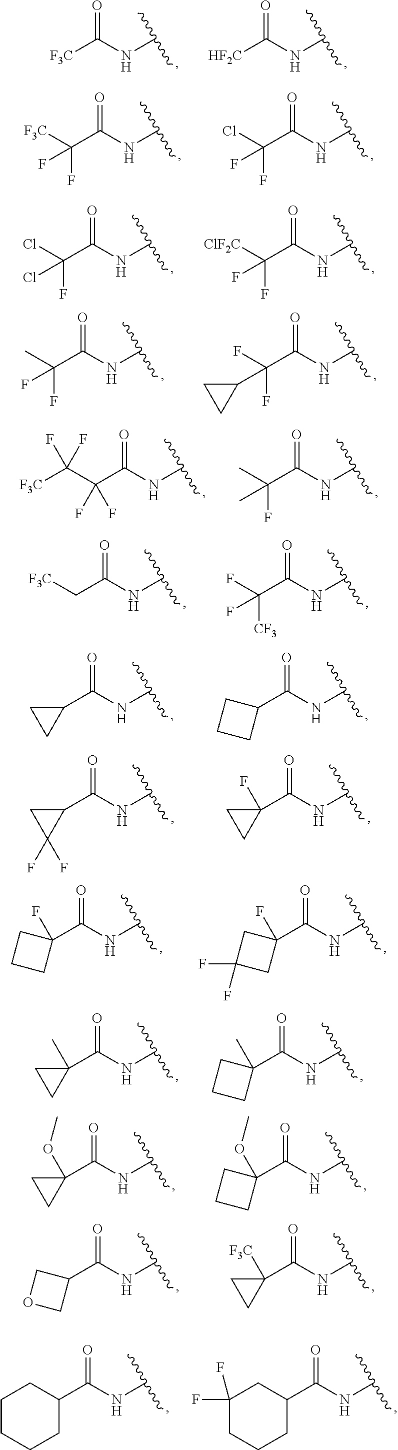

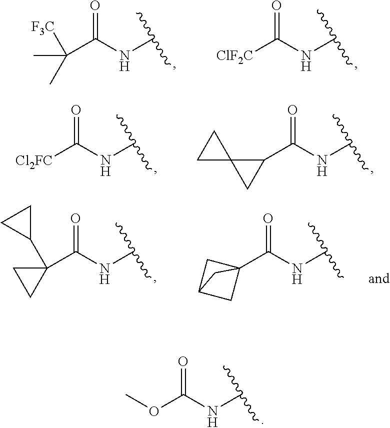

82. The compound of claim 1, wherein R³ is selected from the group consisting of:

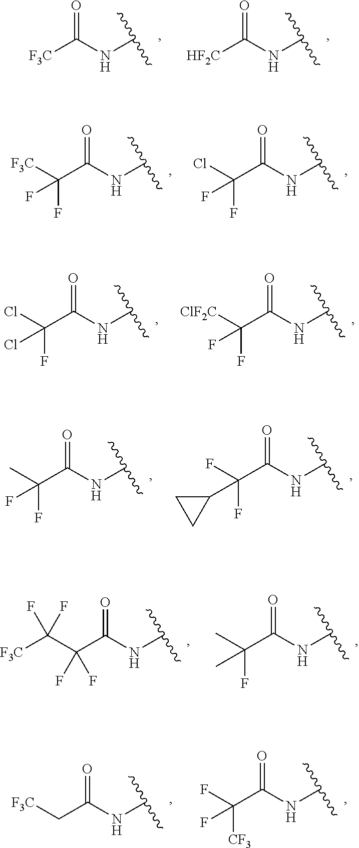

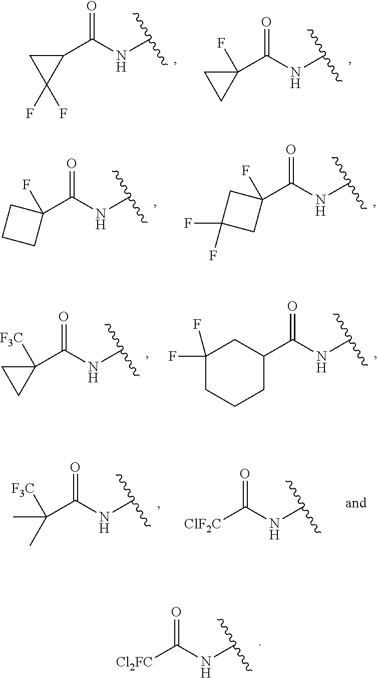

83. The compound of claim 1, wherein R³ is selected from the group consisting of: 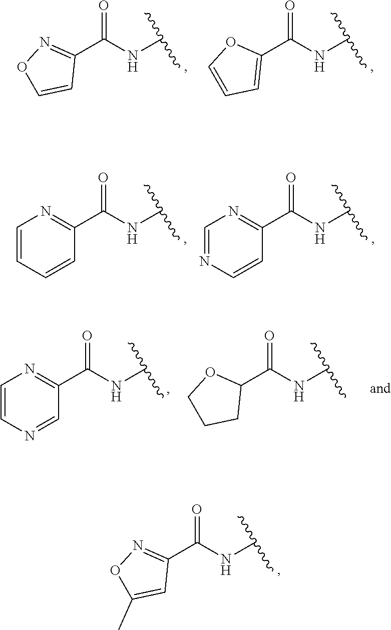
wherein each moiety is unsubstituted or substituted.

84. The compound of claim 1, wherein the compound of Formula (I), or a pharmaceutically acceptable salt thereof, is selected from the group consisting of: 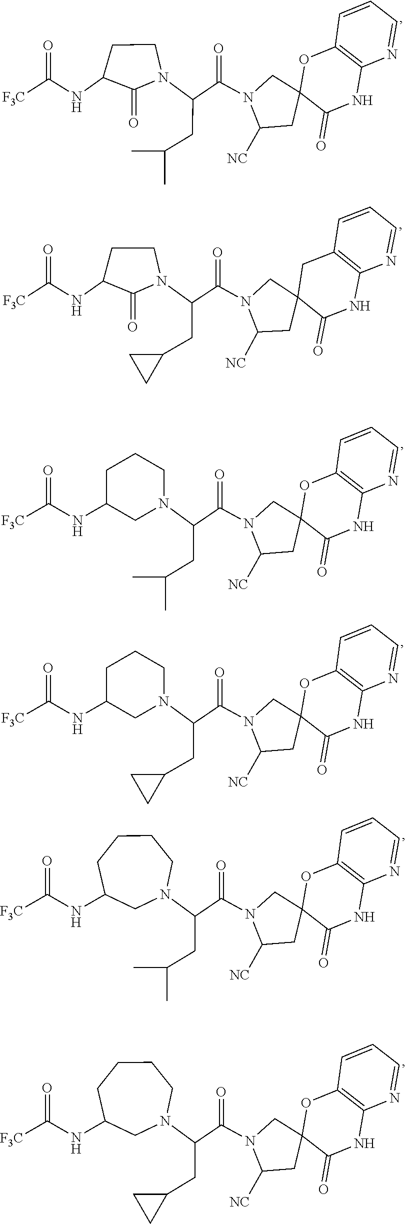 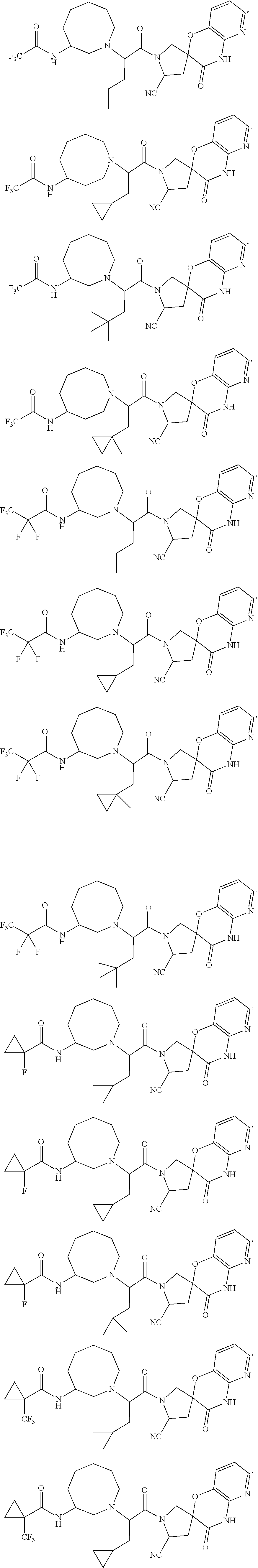 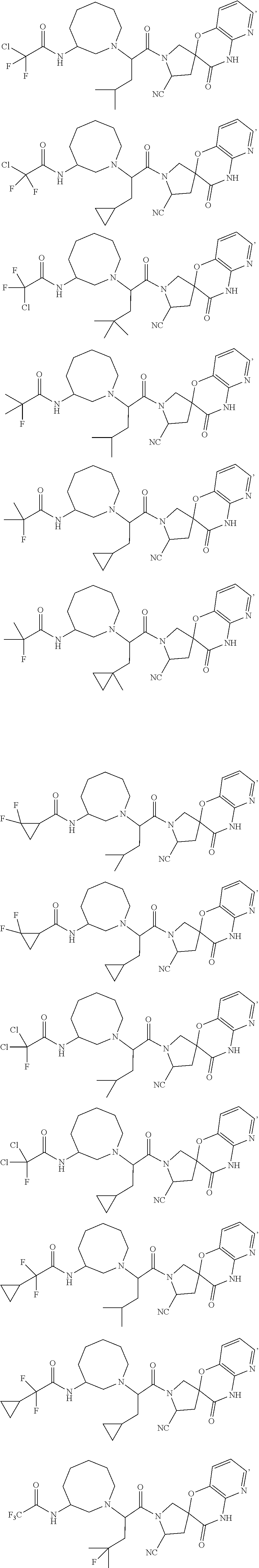 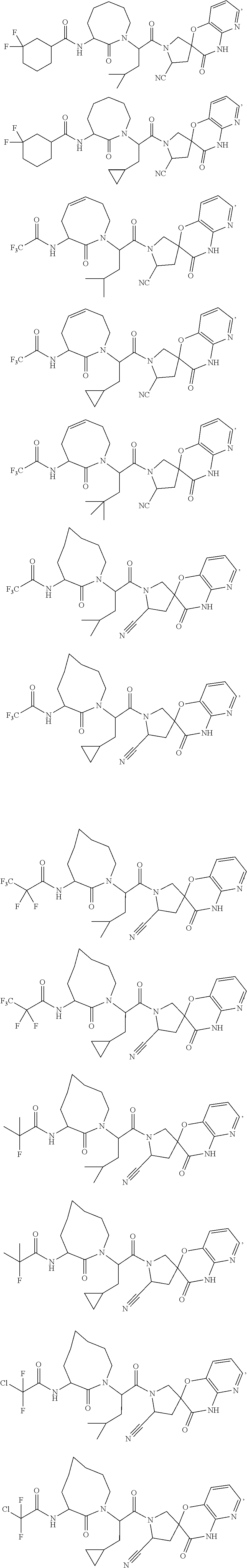 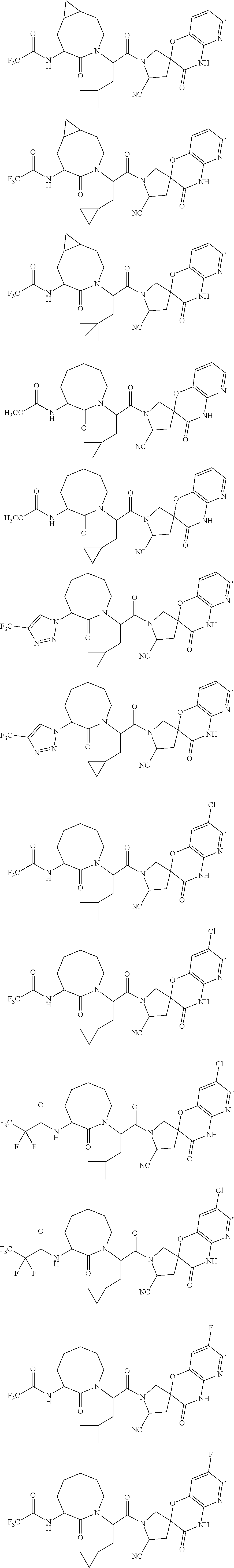 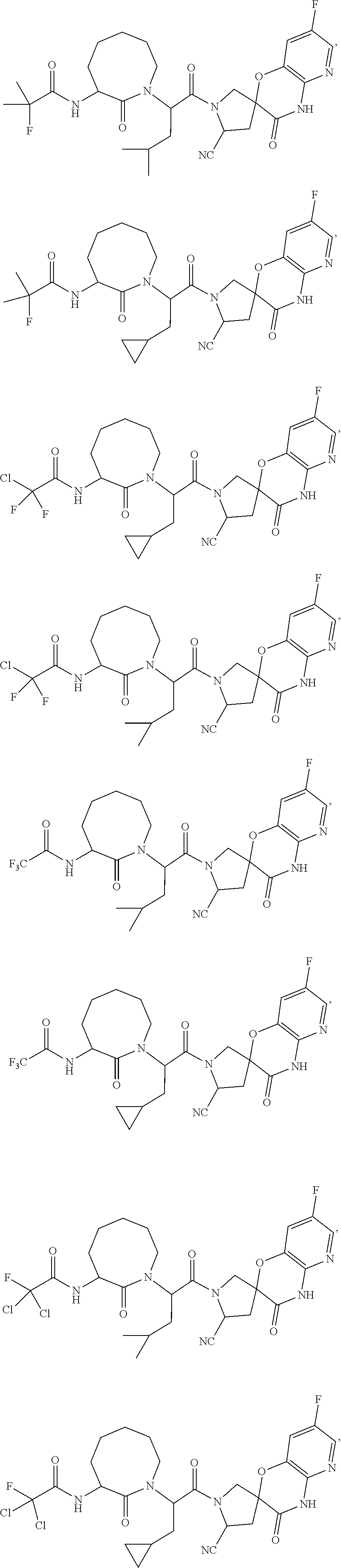 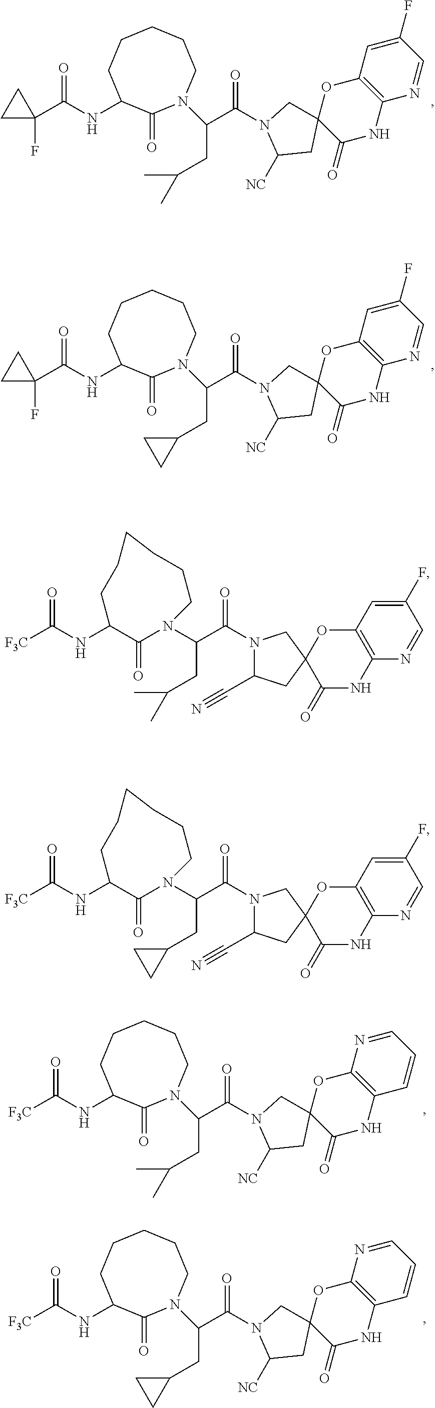 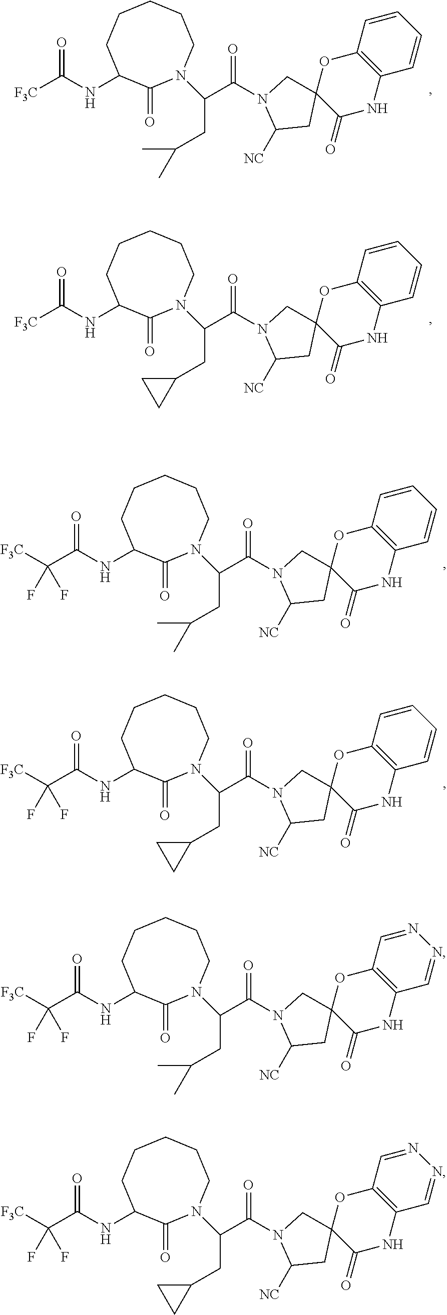 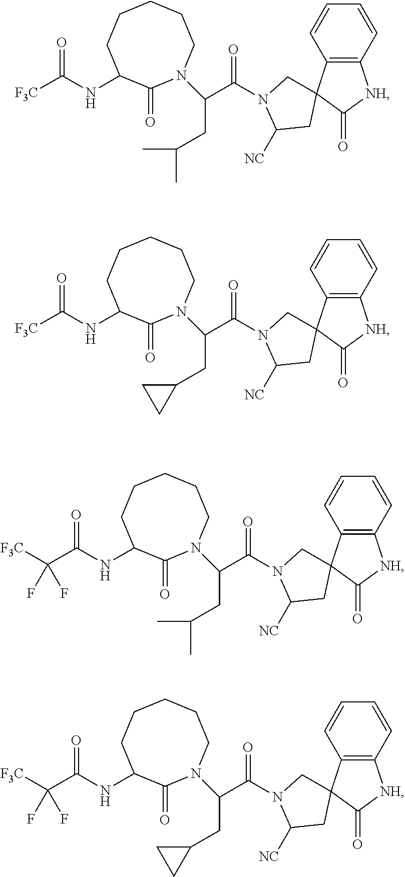 [SMILES: CC(C)CC(C(=O)N1CC2(CC1C#N)C(=O)Nc1ccccc12)N1CCCCCC(NC(=O)C(F)(F)F)C1=O ; N#CC1CC2(CN1C(=O)C(CC1CC1)N1CCCCCC(NC(=O)C(F)(F)F)C1=O)C(=O)Nc1ccccc12 ; CC(C)CC(C(=O)N1CC2(CC1C#N)C(=O)Nc1ccccc12)N1CCCCCC(NC(=O)C(F)(F)C(F)(F)F)C1=O ; N#CC1CC2(CN1C(=O)C(CC1CC1)N1CCCCCC(NC(=O)C(F)(F)C(F)(F)F)C1=O)C(=O)Nc1ccccc12] 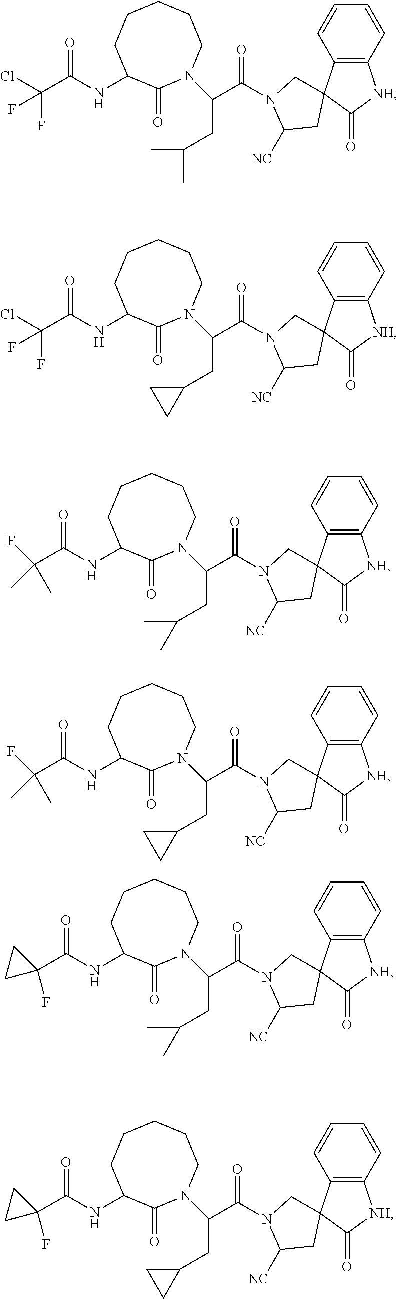 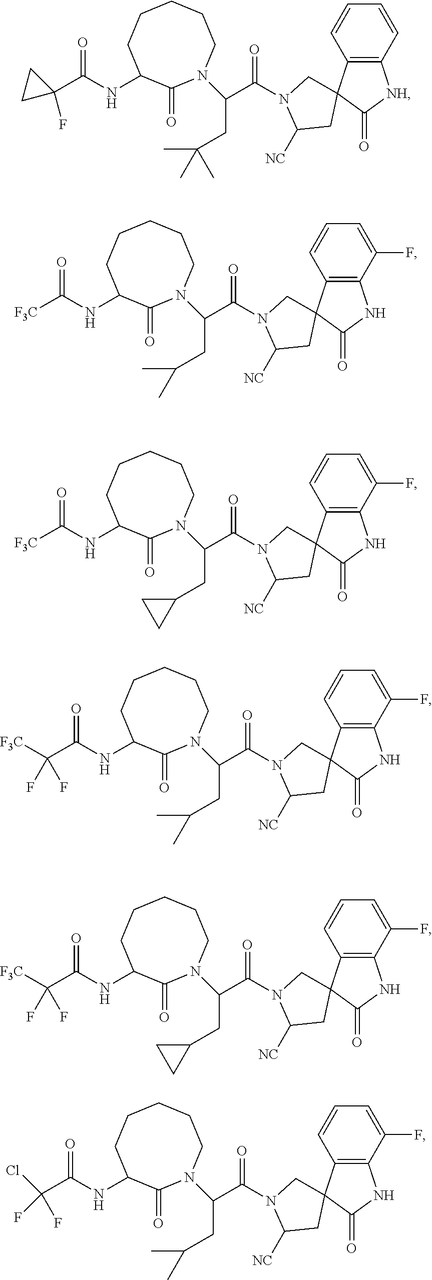 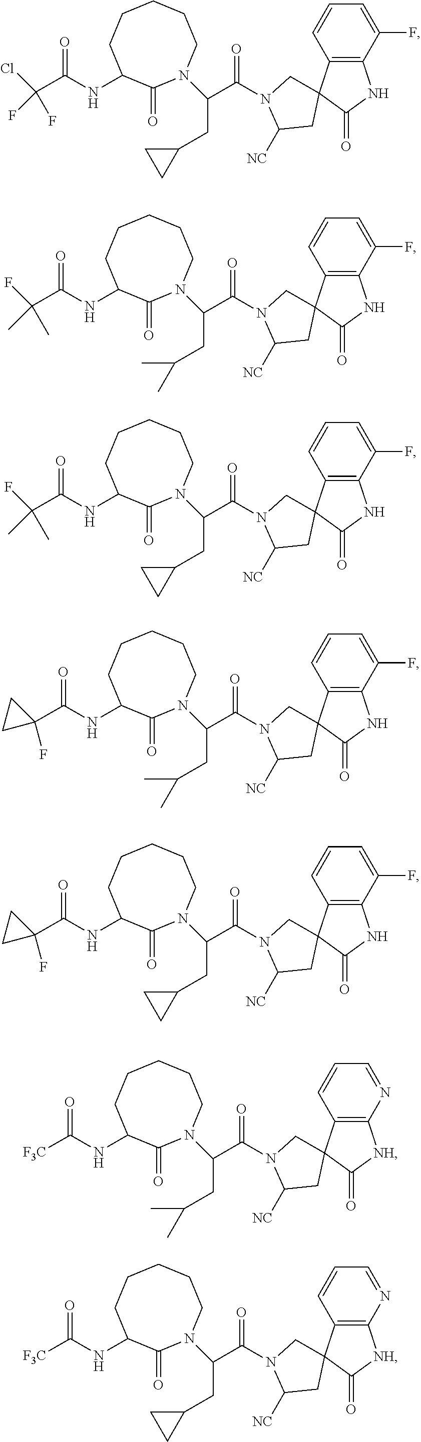 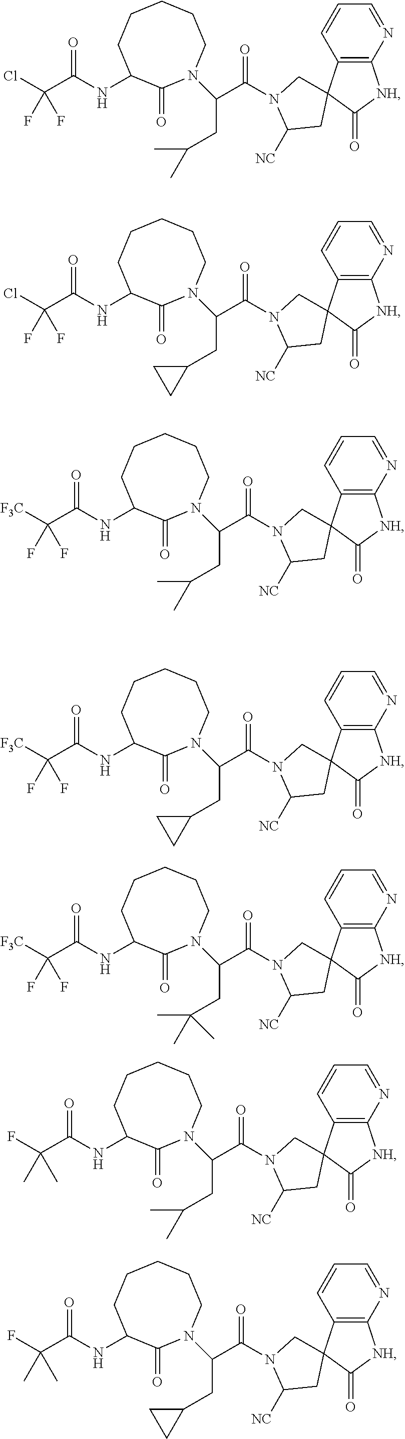 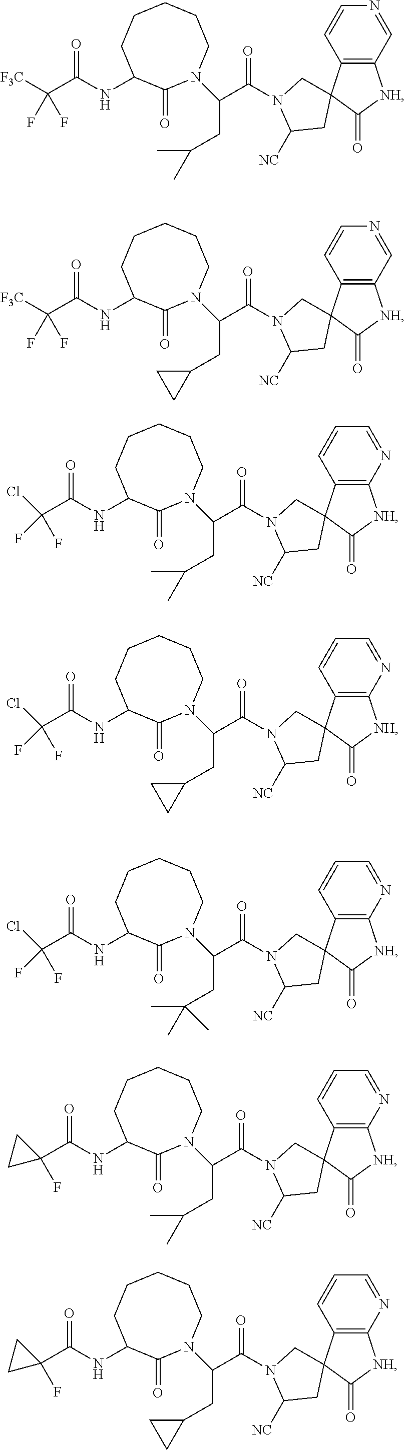 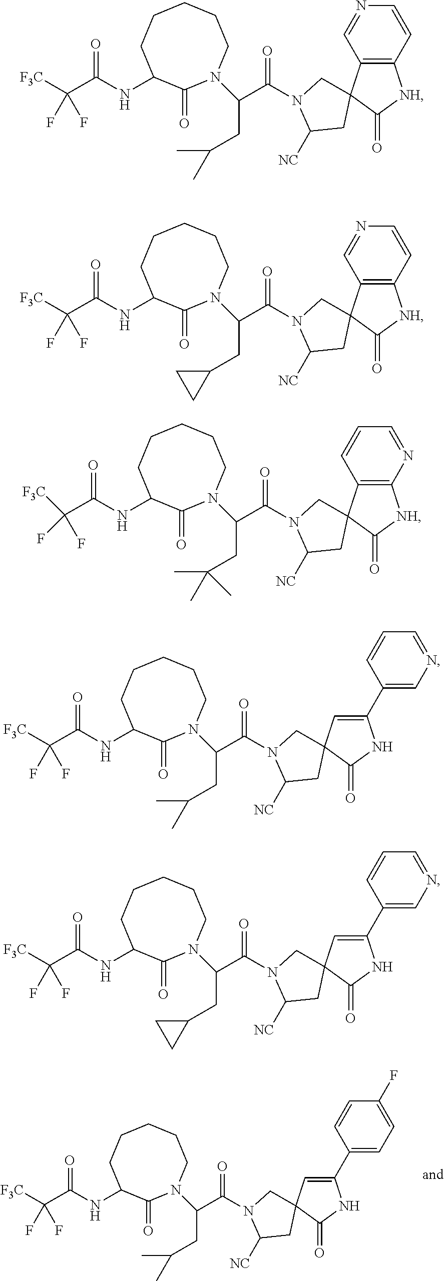 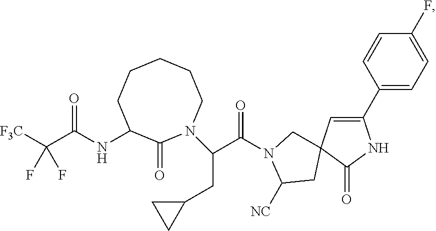 [SMILES: N#CC1CC2(C=C(c3ccc(F)cc3)NC2=O)CN1C(=O)C(CC1CC1)N1CCCCCC(NC(=O)C(F)(F)C(F)(F)F)C1=O]
or a pharmaceutically acceptable salt of any of the foregoing.

85. The compound of claim 1, wherein the compound is selected from the group consisting of: 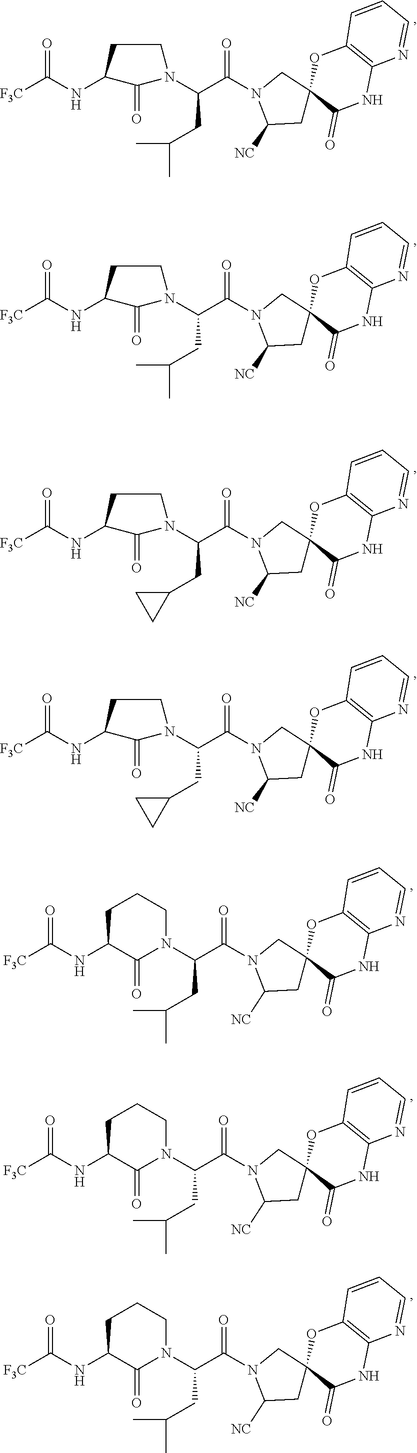 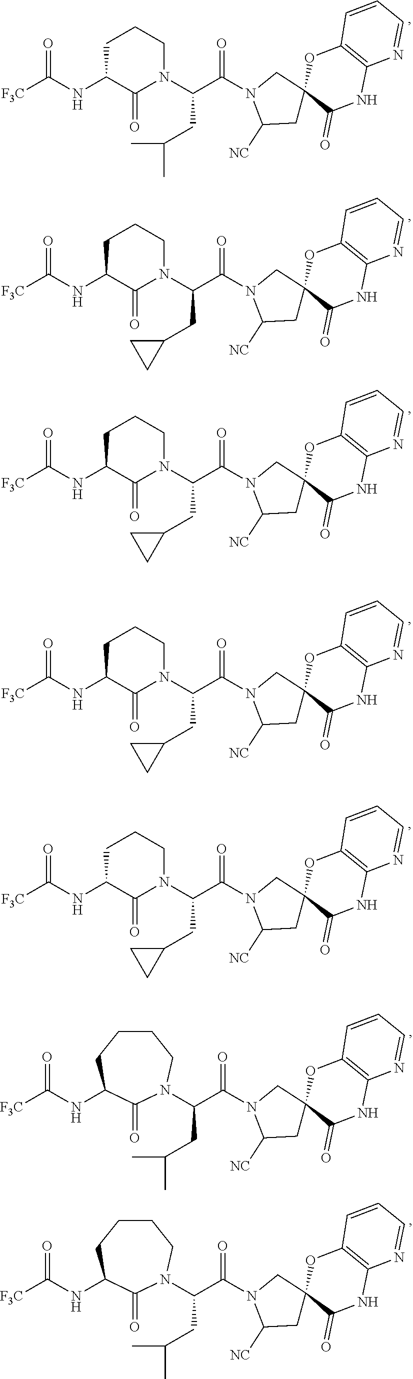 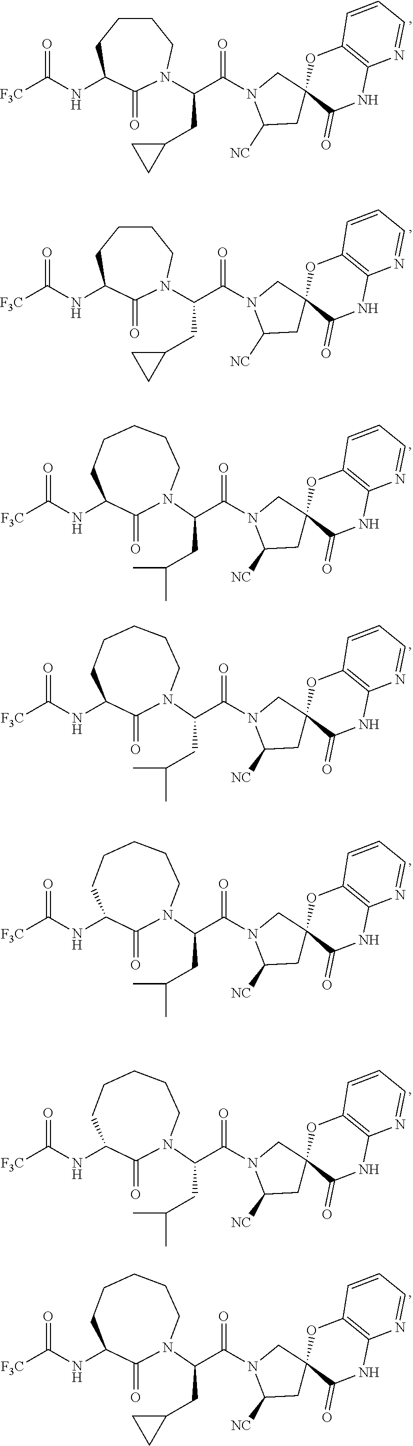 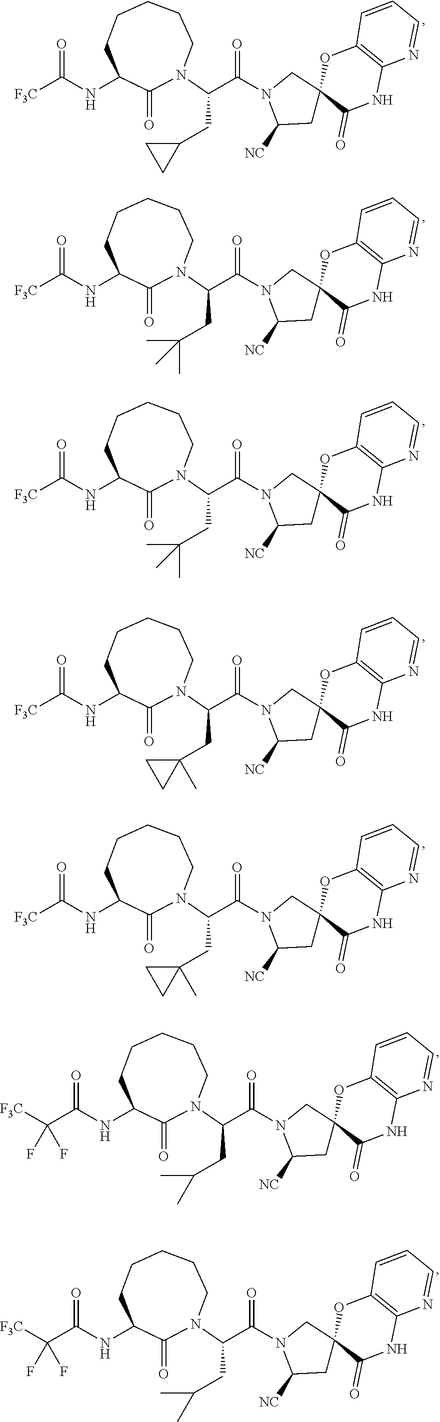 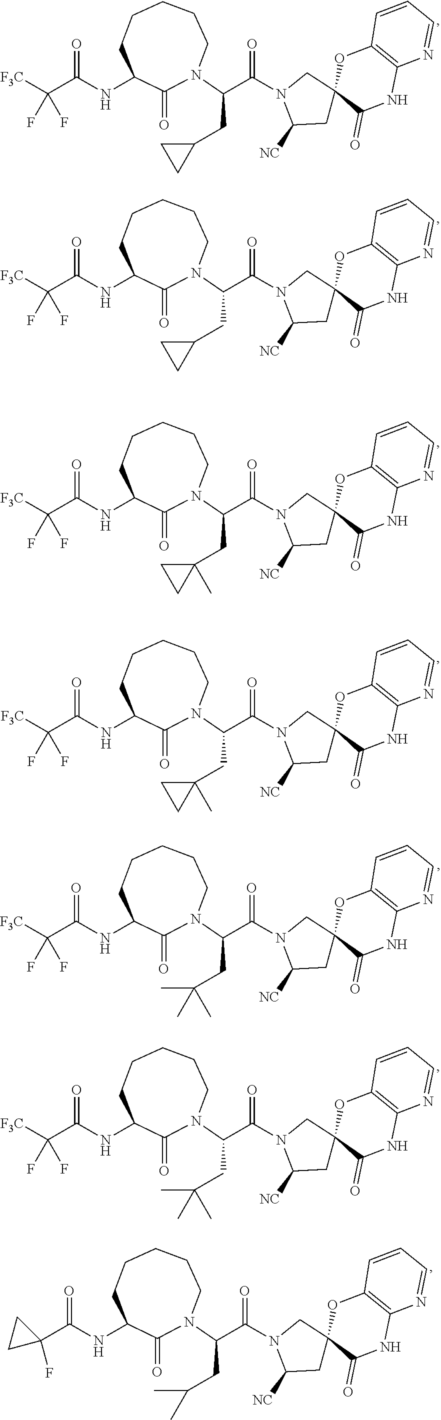 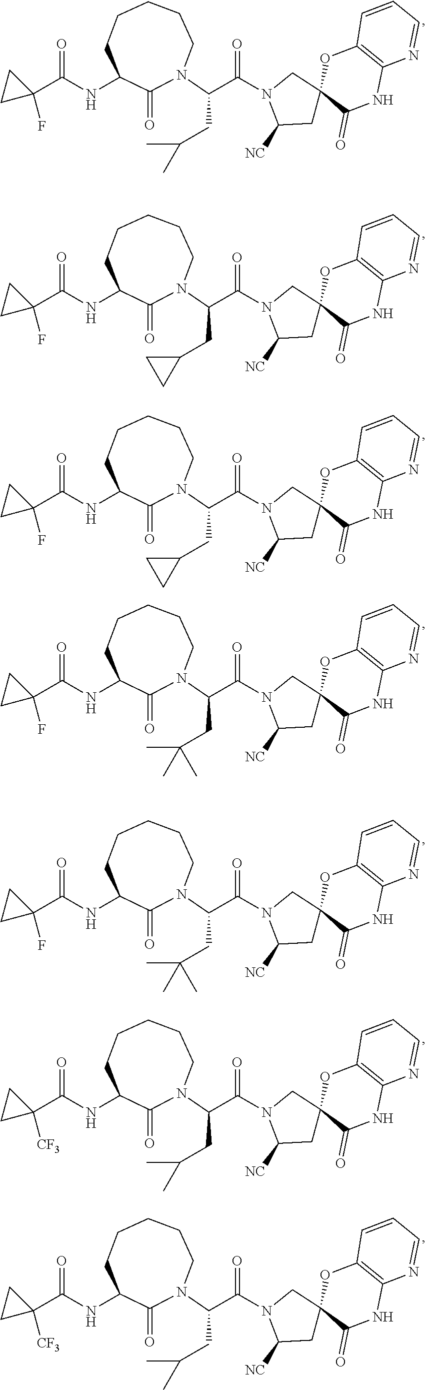 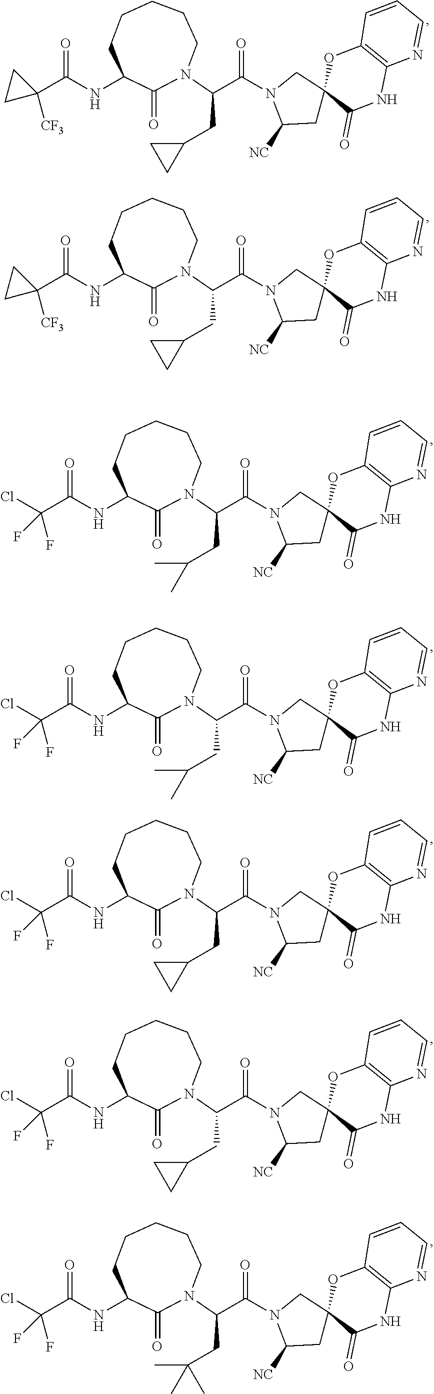 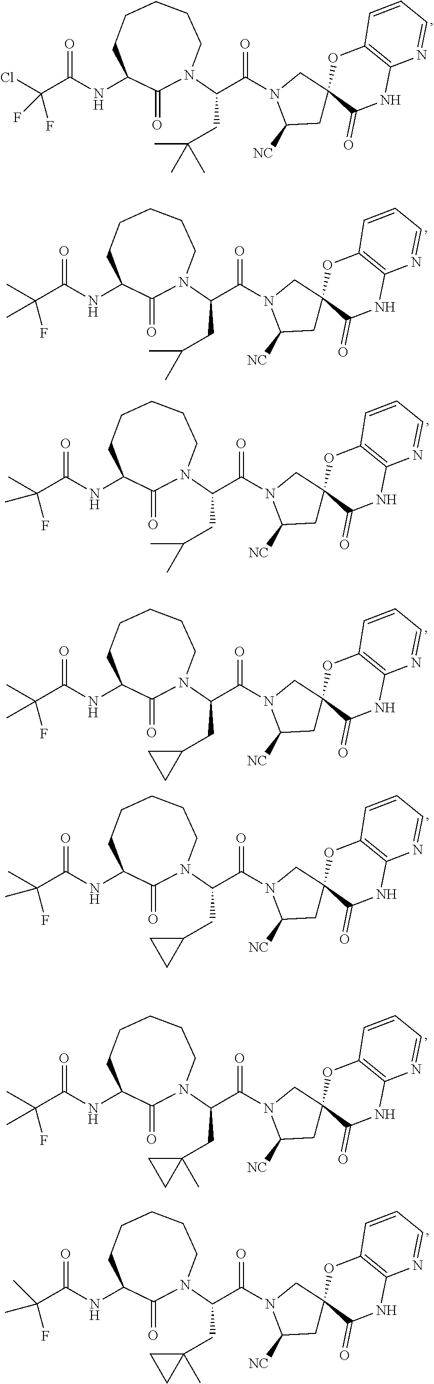 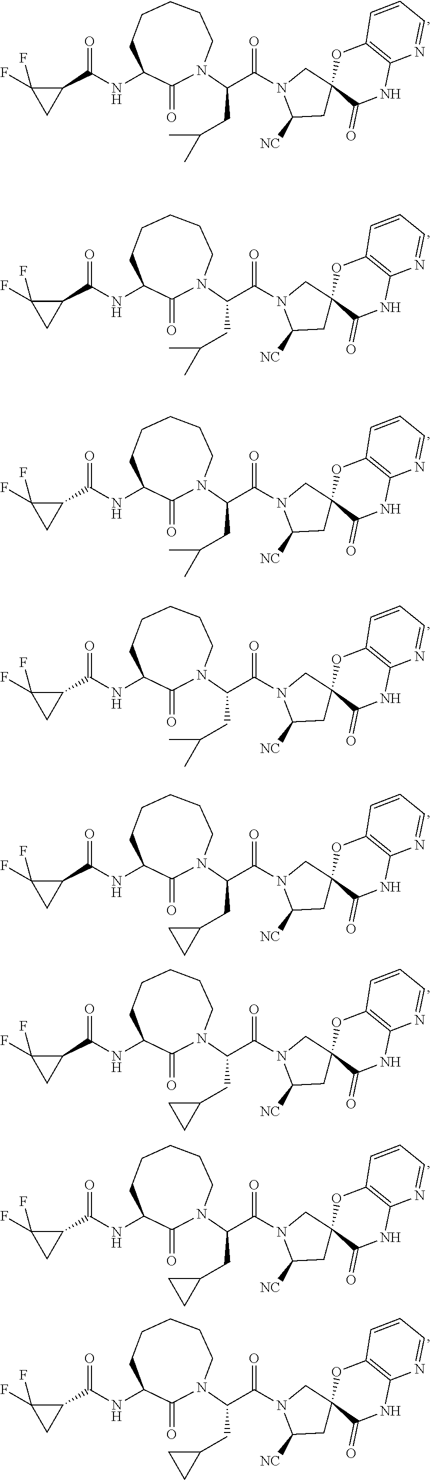 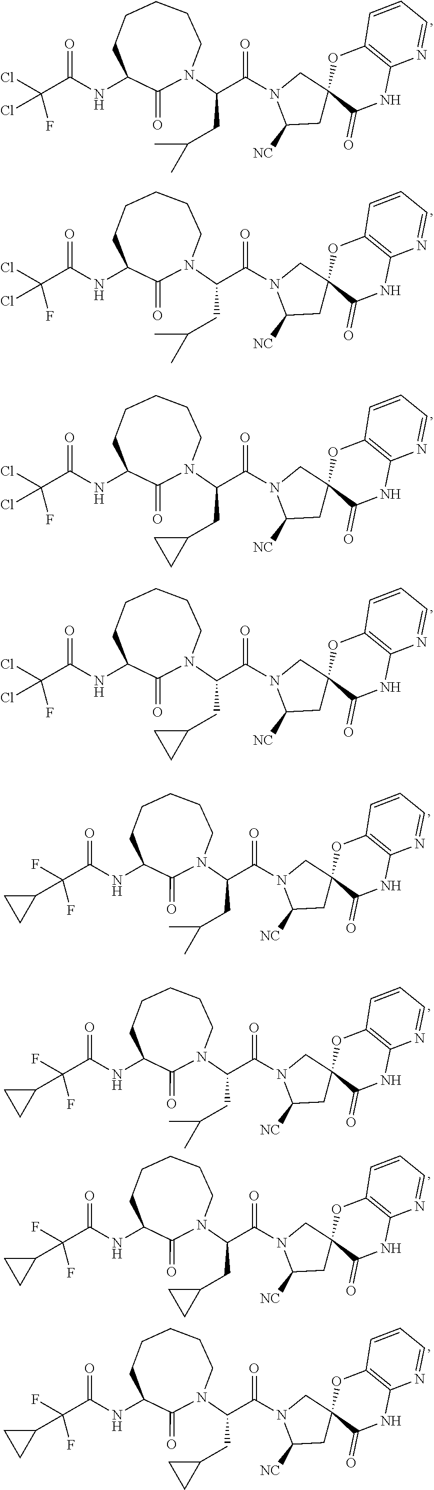 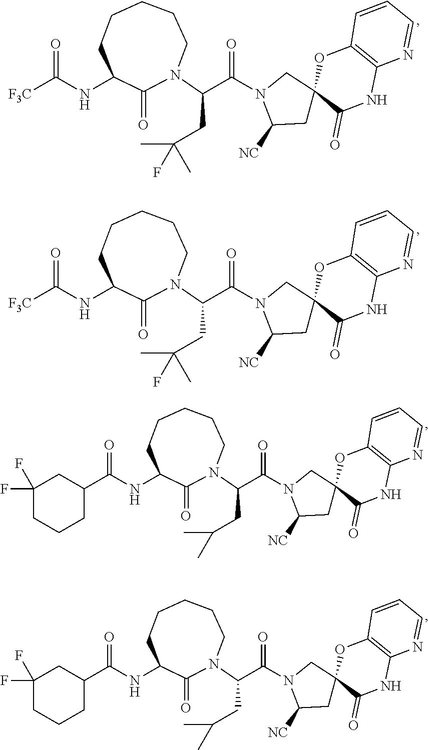 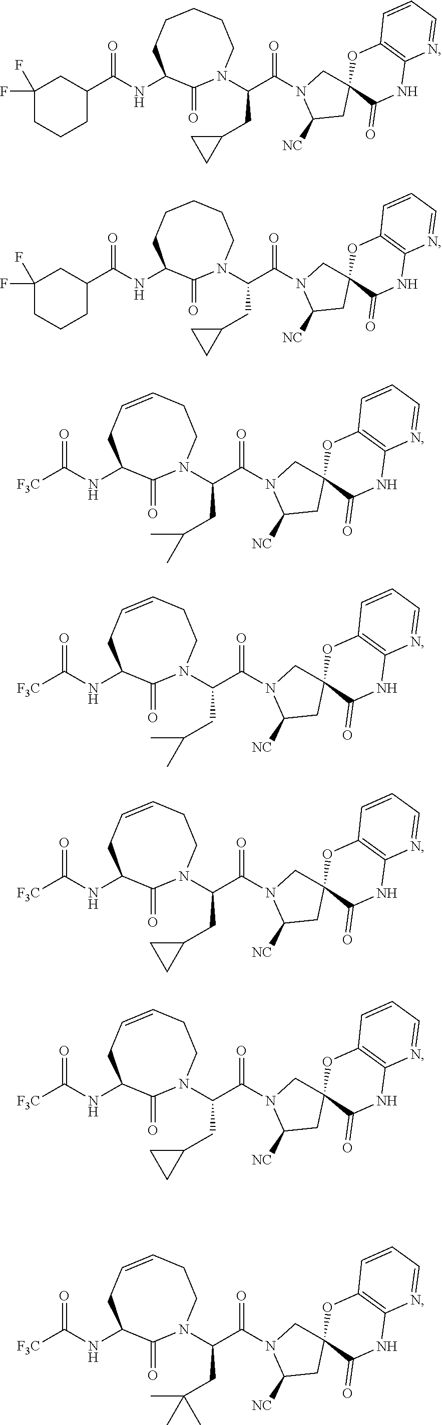 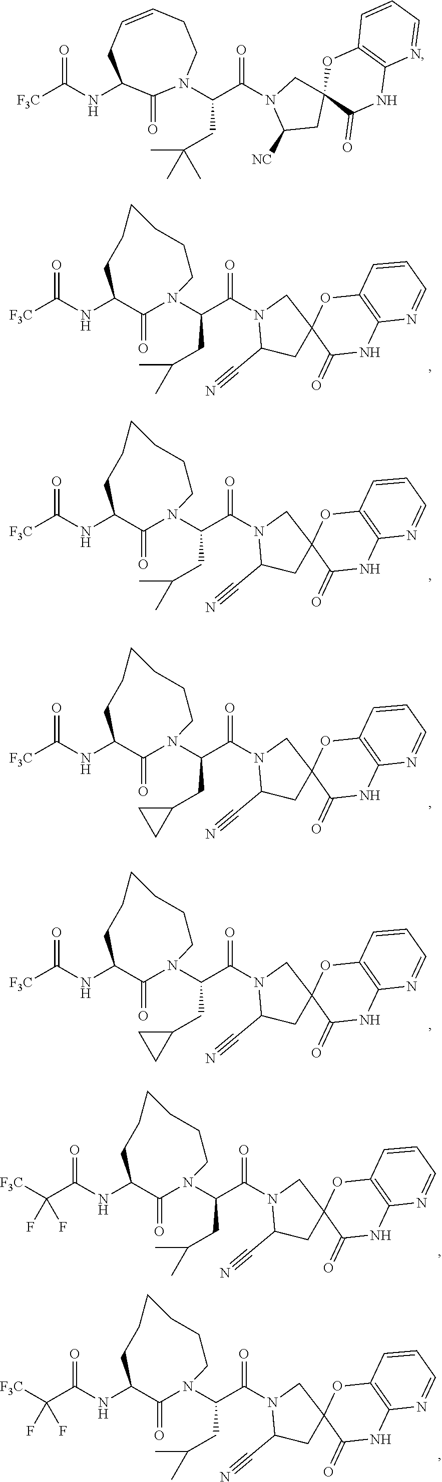 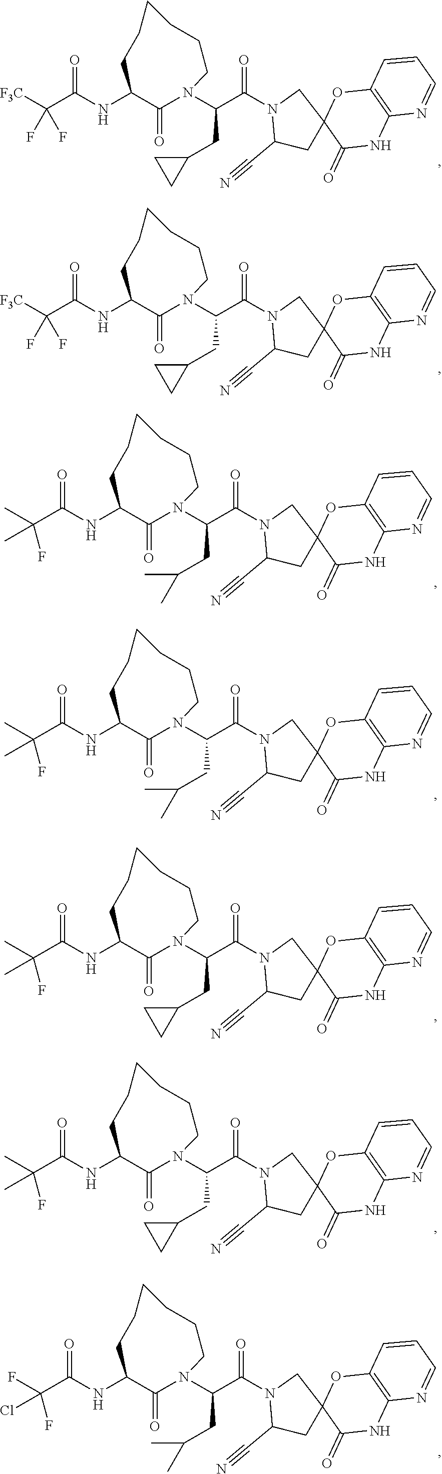 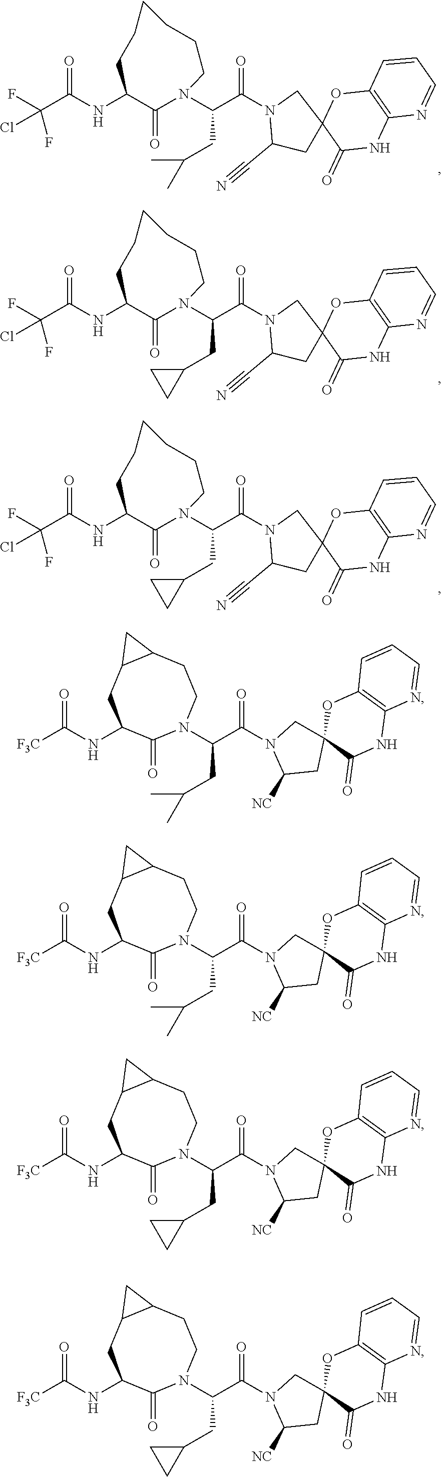 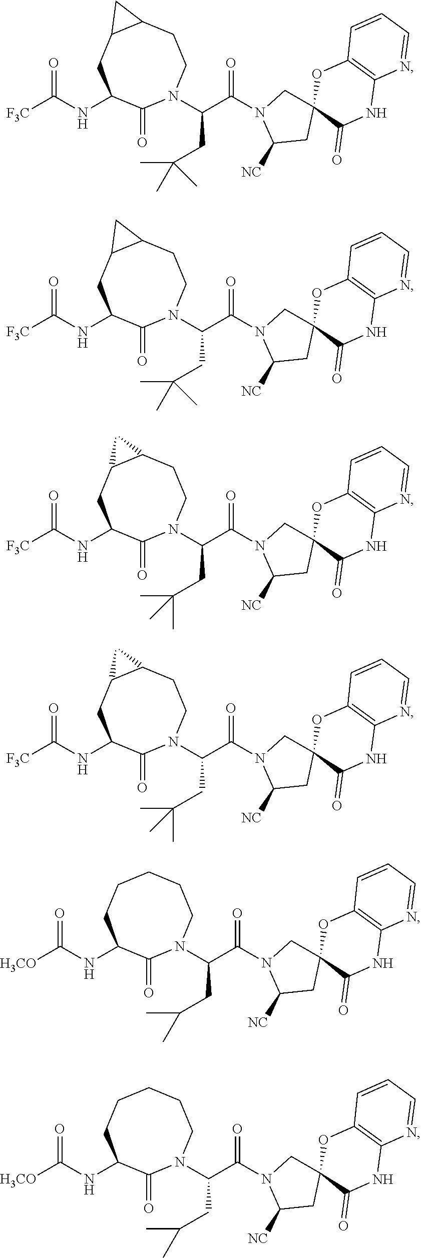 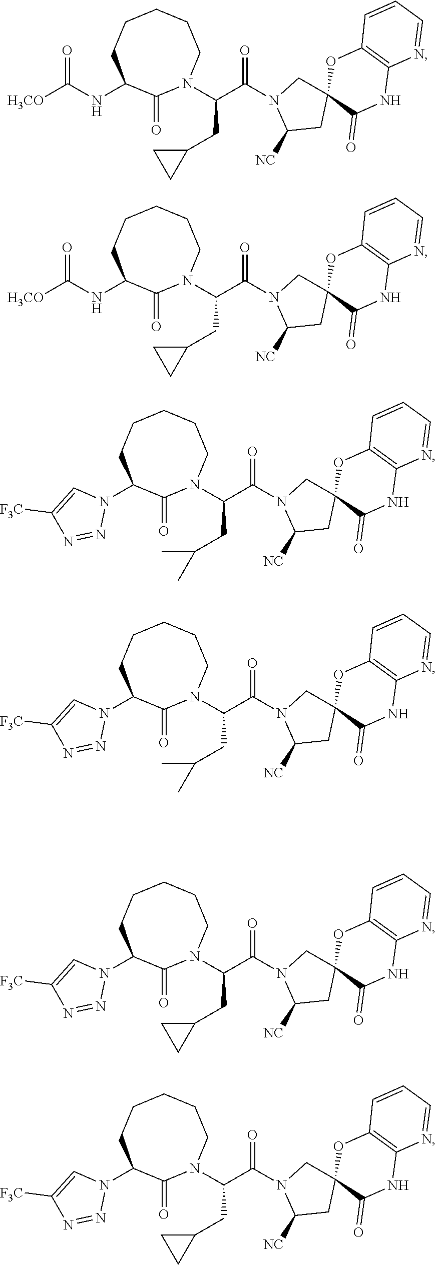 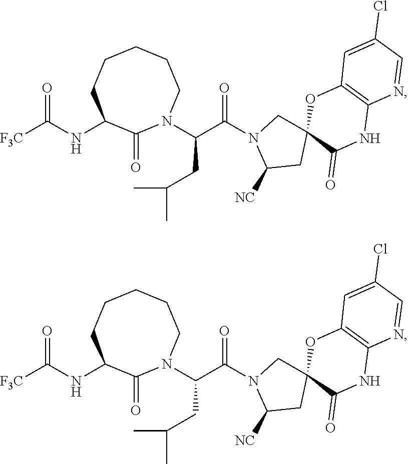 [SMILES: CC(C)C[C@H](C(=O)N1C[C@@]2(C[C@H]1C#N)Oc1cc(Cl)cnc1NC2=O)N1CCCCC[C@H](NC(=O)C(F)(F)F)C1=O ; CC(C)C[C@@H](C(=O)N1C[C@@]2(C[C@H]1C#N)Oc1cc(Cl)cnc1NC2=O)N1CCCCC[C@H](NC(=O)C(F)(F)F)C1=O] 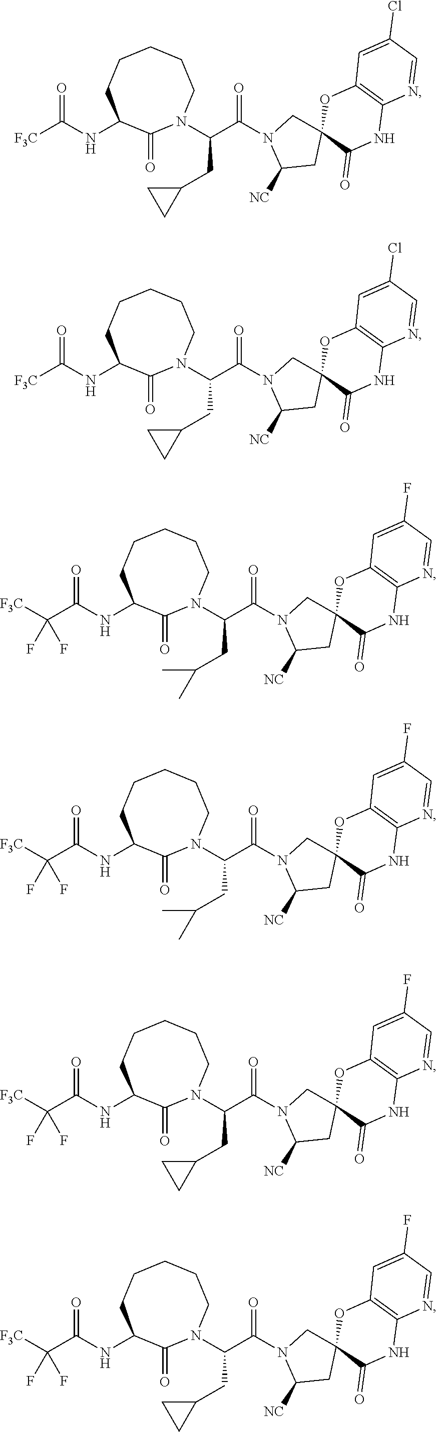 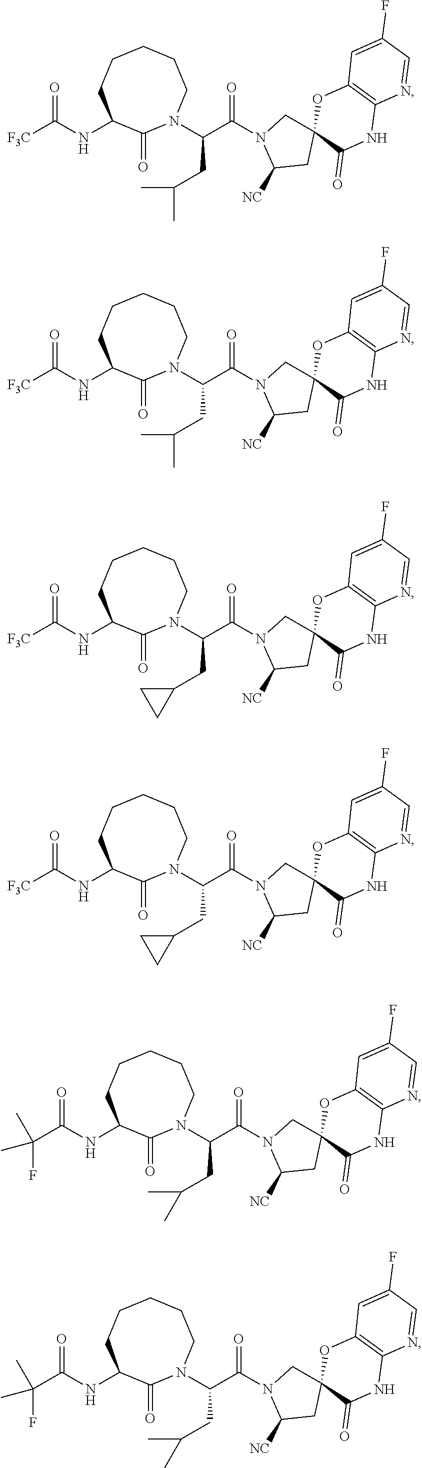 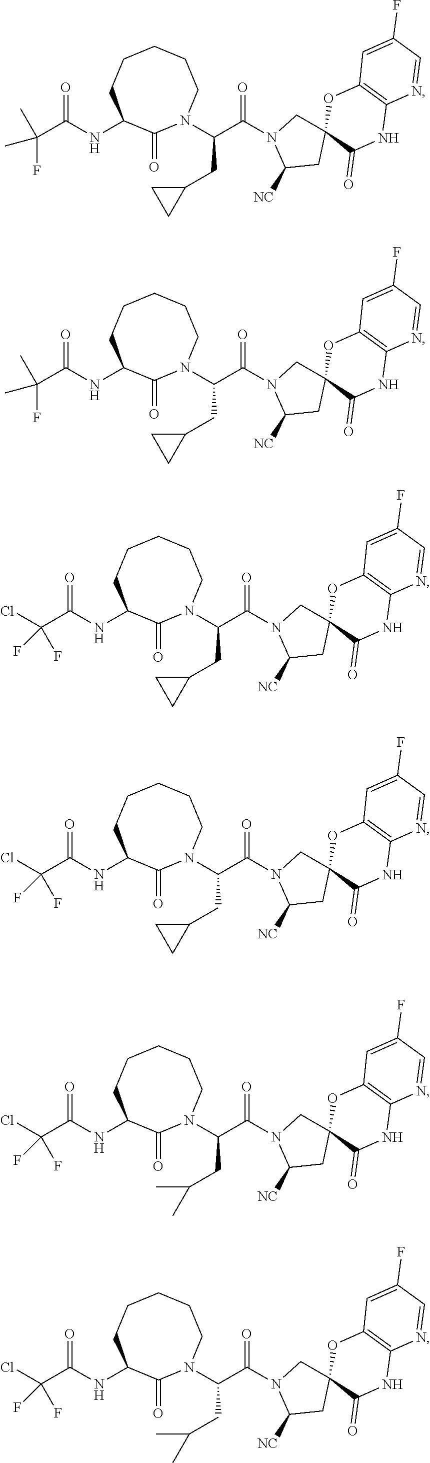 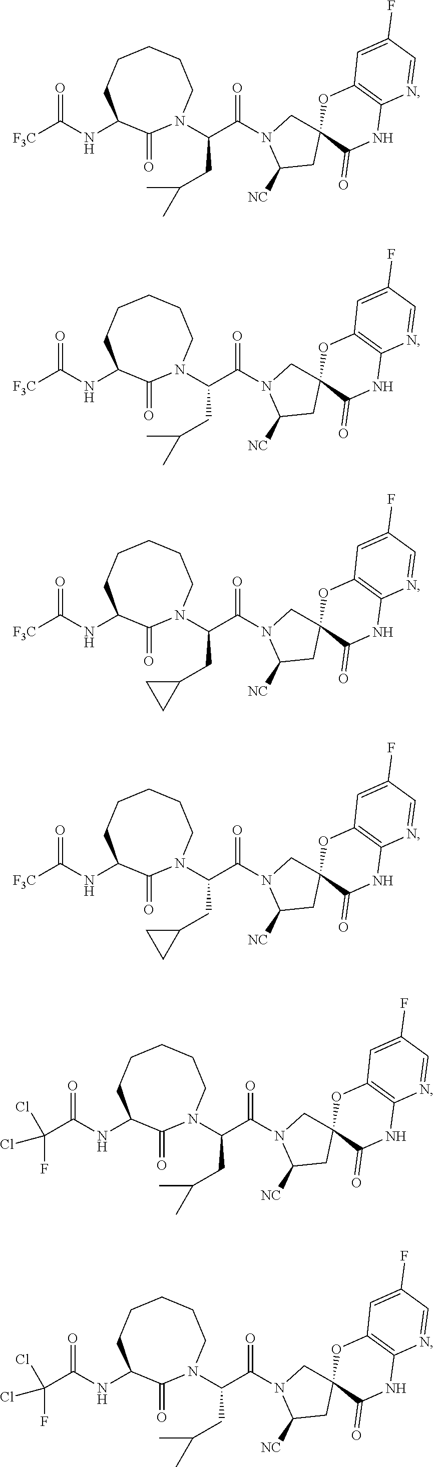 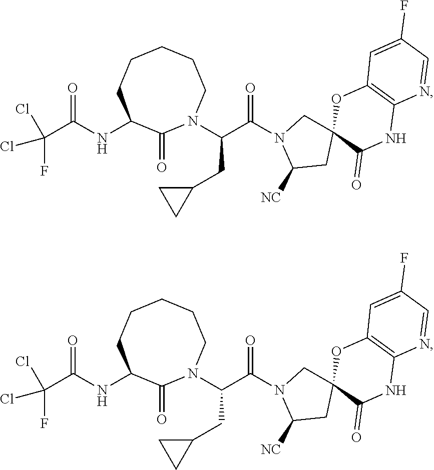 [SMILES: N#C[C@@H]1C[C@]2(CN1C(=O)[C@@H](CC1CC1)N1CCCCC[C@H](NC(=O)C(F)(Cl)Cl)C1=O)Oc1cc(F)cnc1NC2=O ; N#C[C@@H]1C[C@]2(CN1C(=O)[C@H](CC1CC1)N1CCCCC[C@H](NC(=O)C(F)(Cl)Cl)C1=O)Oc1cc(F)cnc1NC2=O] 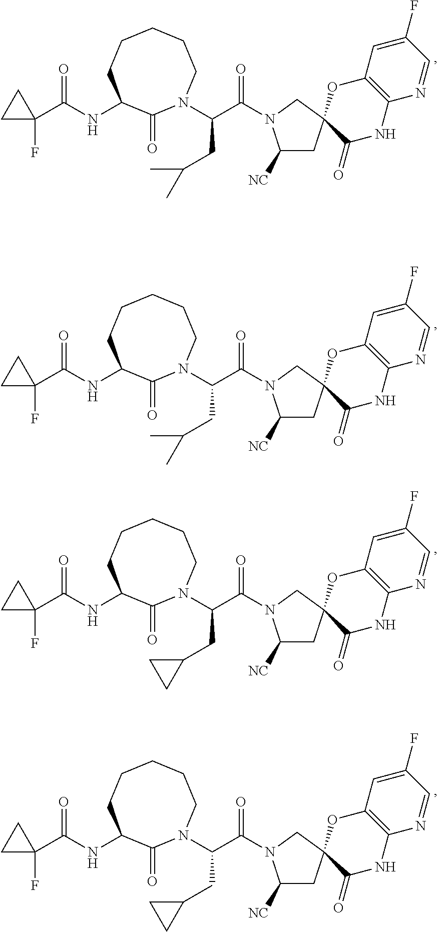 [SMILES: CC(C)C[C@H](C(=O)N1C[C@@]2(C[C@H]1C#N)Oc1cc(F)cnc1NC2=O)N1CCCCC[C@H](NC(=O)C2(F)CC2)C1=O ; CC(C)C[C@@H](C(=O)N1C[C@@]2(C[C@H]1C#N)Oc1cc(F)cnc1NC2=O)N1CCCCC[C@H](NC(=O)C2(F)CC2)C1=O ; N#C[C@@H]1C[C@]2(CN1C(=O)[C@@H](CC1CC1)N1CCCCC[C@H](NC(=O)C3(F)CC3)C1=O)Oc1cc(F)cnc1NC2=O ; N#C[C@@H]1C[C@]2(CN1C(=O)[C@H](CC1CC1)N1CCCCC[C@H](NC(=O)C3(F)CC3)C1=O)Oc1cc(F)cnc1NC2=O] 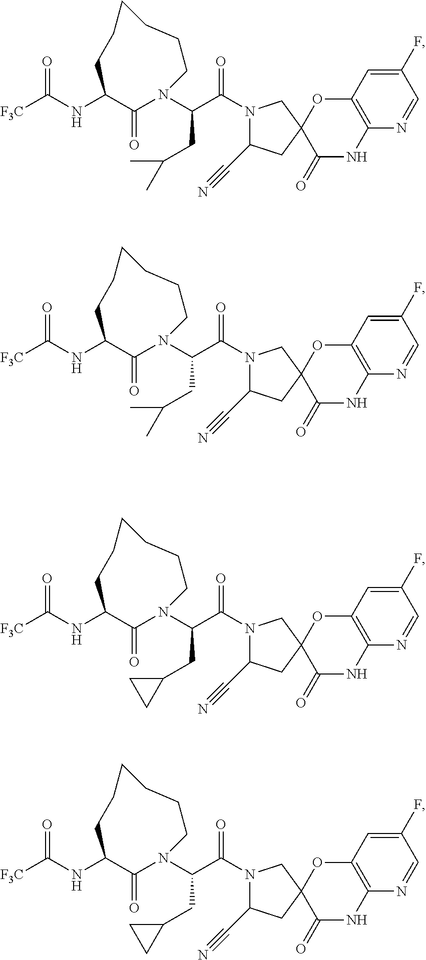 [SMILES: CC(C)C[C@H](C(=O)N1CC2(CC1C#N)Oc1cc(F)cnc1NC2=O)N1CCCCCC[C@H](NC(=O)C(F)(F)F)C1=O ; CC(C)C[C@@H](C(=O)N1CC2(CC1C#N)Oc1cc(F)cnc1NC2=O)N1CCCCCC[C@H](NC(=O)C(F)(F)F)C1=O ; N#CC1CC2(CN1C(=O)[C@@H](CC1CC1)N1CCCCCC[C@H](NC(=O)C(F)(F)F)C1=O)Oc1cc(F)cnc1NC2=O ; N#CC1CC2(CN1C(=O)[C@H](CC1CC1)N1CCCCCC[C@H](NC(=O)C(F)(F)F)C1=O)Oc1cc(F)cnc1NC2=O] 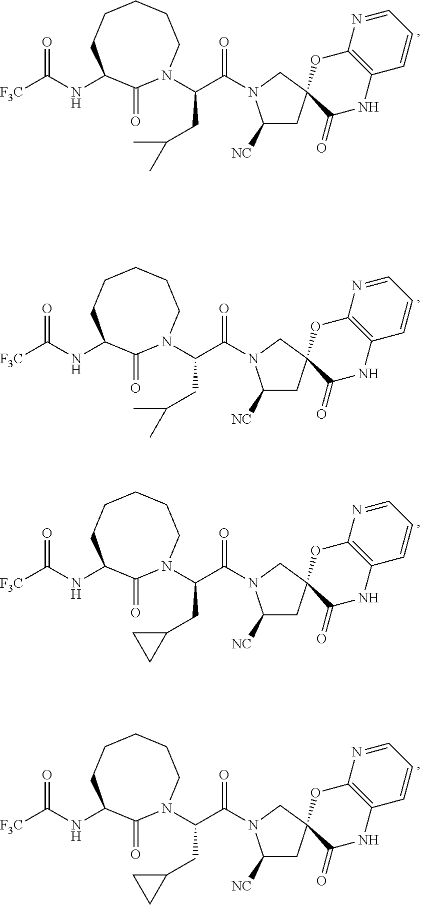 [SMILES: CC(C)C[C@H](C(=O)N1C[C@@]2(C[C@H]1C#N)Oc1ncccc1NC2=O)N1CCCCC[C@H](NC(=O)C(F)(F)F)C1=O ; CC(C)C[C@@H](C(=O)N1C[C@@]2(C[C@H]1C#N)Oc1ncccc1NC2=O)N1CCCCC[C@H](NC(=O)C(F)(F)F)C1=O ; N#C[C@@H]1C[C@]2(CN1C(=O)[C@@H](CC1CC1)N1CCCCC[C@H](NC(=O)C(F)(F)F)C1=O)Oc1ncccc1NC2=O ; N#C[C@@H]1C[C@]2(CN1C(=O)[C@H](CC1CC1)N1CCCCC[C@H](NC(=O)C(F)(F)F)C1=O)Oc1ncccc1NC2=O] 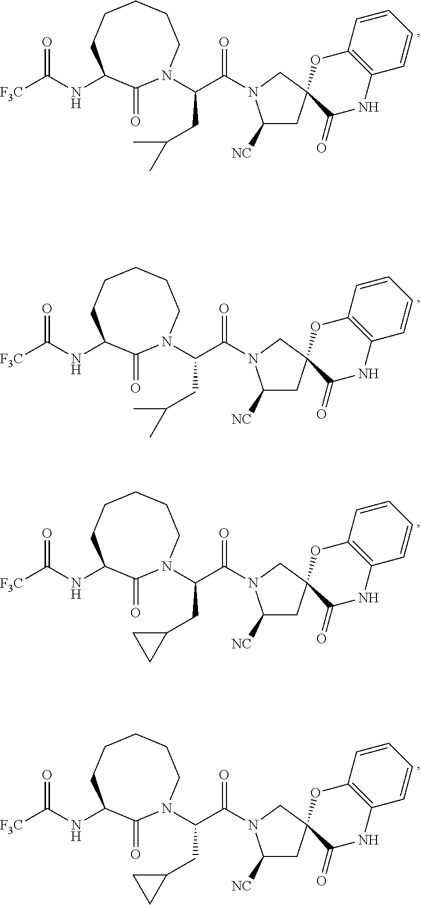 [SMILES: CC(C)C[C@H](C(=O)N1C[C@@]2(C[C@H]1C#N)Oc1ccccc1NC2=O)N1CCCCC[C@H](NC(=O)C(F)(F)F)C1=O ; CC(C)C[C@@H](C(=O)N1C[C@@]2(C[C@H]1C#N)Oc1ccccc1NC2=O)N1CCCCC[C@H](NC(=O)C(F)(F)F)C1=O ; N#C[C@@H]1C[C@]2(CN1C(=O)[C@@H](CC1CC1)N1CCCCC[C@H](NC(=O)C(F)(F)F)C1=O)Oc1ccccc1NC2=O ; N#C[C@@H]1C[C@]2(CN1C(=O)[C@H](CC1CC1)N1CCCCC[C@H](NC(=O)C(F)(F)F)C1=O)Oc1ccccc1NC2=O] 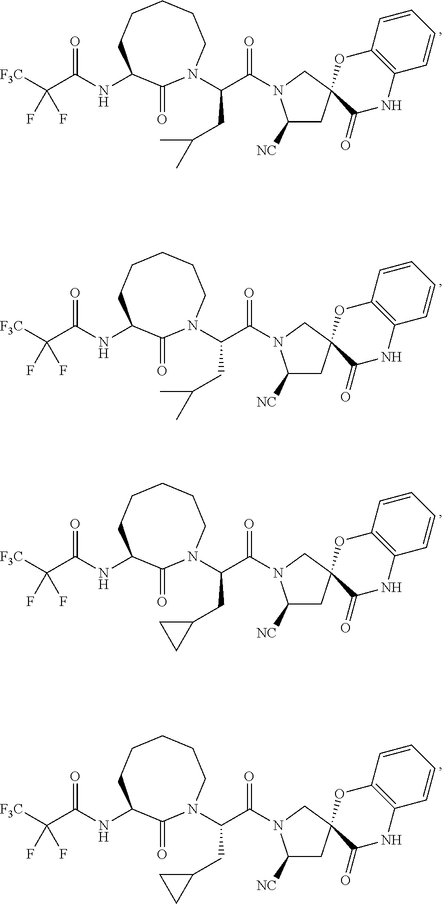 [SMILES: CC(C)C[C@H](C(=O)N1C[C@@]2(C[C@H]1C#N)Oc1ccccc1NC2=O)N1CCCCC[C@H](NC(=O)C(F)(F)C(F)(F)F)C1=O ; CC(C)C[C@@H](C(=O)N1C[C@@]2(C[C@H]1C#N)Oc1ccccc1NC2=O)N1CCCCC[C@H](NC(=O)C(F)(F)C(F)(F)F)C1=O ; N#C[C@@H]1C[C@]2(CN1C(=O)[C@@H](CC1CC1)N1CCCCC[C@H](NC(=O)C(F)(F)C(F)(F)F)C1=O)Oc1ccccc1NC2=O ; N#C[C@@H]1C[C@]2(CN1C(=O)[C@H](CC1CC1)N1CCCCC[C@H](NC(=O)C(F)(F)C(F)(F)F)C1=O)Oc1ccccc1NC2=O] 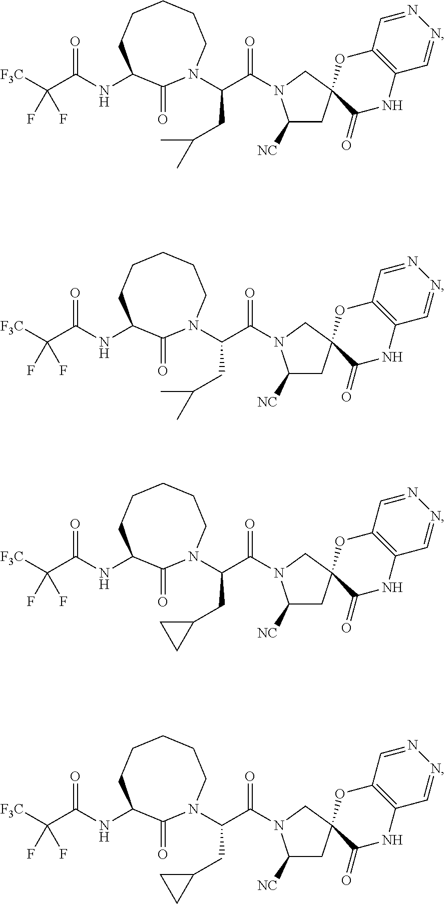 [SMILES: CC(C)C[C@H](C(=O)N1C[C@@]2(C[C@H]1C#N)Oc1cnncc1NC2=O)N1CCCCC[C@H](NC(=O)C(F)(F)C(F)(F)F)C1=O ; CC(C)C[C@@H](C(=O)N1C[C@@]2(C[C@H]1C#N)Oc1cnncc1NC2=O)N1CCCCC[C@H](NC(=O)C(F)(F)C(F)(F)F)C1=O ; N#C[C@@H]1C[C@]2(CN1C(=O)[C@@H](CC1CC1)N1CCCCC[C@H](NC(=O)C(F)(F)C(F)(F)F)C1=O)Oc1cnncc1NC2=O ; N#C[C@@H]1C[C@]2(CN1C(=O)[C@H](CC1CC1)N1CCCCC[C@H](NC(=O)C(F)(F)C(F)(F)F)C1=O)Oc1cnncc1NC2=O] 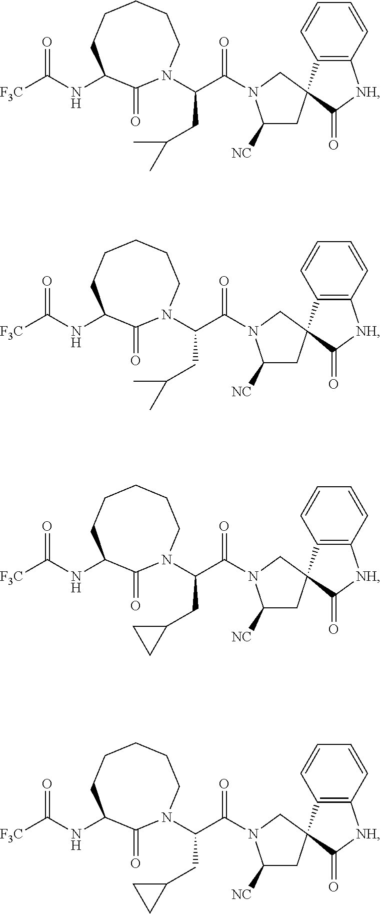 [SMILES: CC(C)C[C@H](C(=O)N1C[C@]2(C[C@H]1C#N)C(=O)Nc1ccccc12)N1CCCCC[C@H](NC(=O)C(F)(F)F)C1=O ; CC(C)C[C@@H](C(=O)N1C[C@]2(C[C@H]1C#N)C(=O)Nc1ccccc12)N1CCCCC[C@H](NC(=O)C(F)(F)F)C1=O ; N#C[C@@H]1C[C@@]2(CN1C(=O)[C@@H](CC1CC1)N1CCCCC[C@H](NC(=O)C(F)(F)F)C1=O)C(=O)Nc1ccccc12 ; N#C[C@@H]1C[C@@]2(CN1C(=O)[C@H](CC1CC1)N1CCCCC[C@H](NC(=O)C(F)(F)F)C1=O)C(=O)Nc1ccccc12] 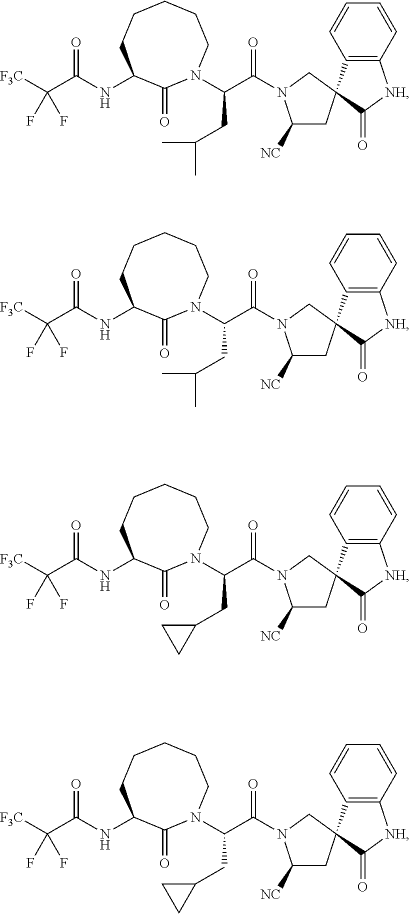 [SMILES: CC(C)C[C@H](C(=O)N1C[C@]2(C[C@H]1C#N)C(=O)Nc1ccccc12)N1CCCCC[C@H](NC(=O)C(F)(F)C(F)(F)F)C1=O ; CC(C)C[C@@H](C(=O)N1C[C@]2(C[C@H]1C#N)C(=O)Nc1ccccc12)N1CCCCC[C@H](NC(=O)C(F)(F)C(F)(F)F)C1=O ; N#C[C@@H]1C[C@@]2(CN1C(=O)[C@@H](CC1CC1)N1CCCCC[C@H](NC(=O)C(F)(F)C(F)(F)F)C1=O)C(=O)Nc1ccccc12 ; N#C[C@@H]1C[C@@]2(CN1C(=O)[C@H](CC1CC1)N1CCCCC[C@H](NC(=O)C(F)(F)C(F)(F)F)C1=O)C(=O)Nc1ccccc12] 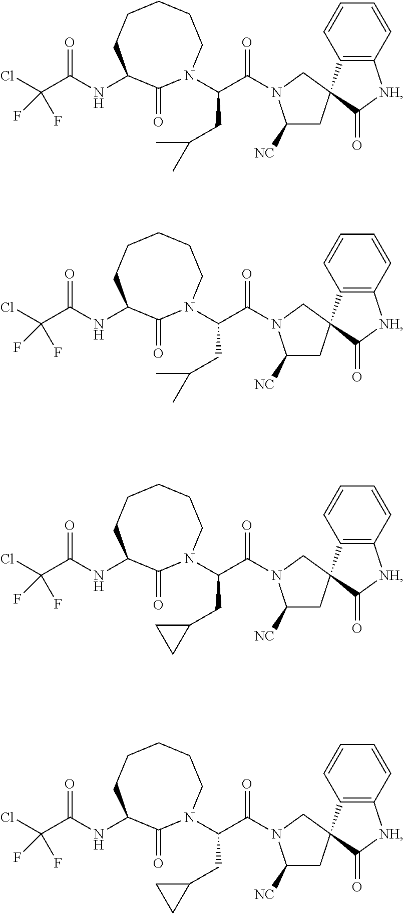 [SMILES: CC(C)C[C@H](C(=O)N1C[C@]2(C[C@H]1C#N)C(=O)Nc1ccccc12)N1CCCCC[C@H](NC(=O)C(F)(F)Cl)C1=O ; CC(C)C[C@@H](C(=O)N1C[C@]2(C[C@H]1C#N)C(=O)Nc1ccccc12)N1CCCCC[C@H](NC(=O)C(F)(F)Cl)C1=O ; N#C[C@@H]1C[C@@]2(CN1C(=O)[C@@H](CC1CC1)N1CCCCC[C@H](NC(=O)C(F)(F)Cl)C1=O)C(=O)Nc1ccccc12 ; N#C[C@@H]1C[C@@]2(CN1C(=O)[C@H](CC1CC1)N1CCCCC[C@H](NC(=O)C(F)(F)Cl)C1=O)C(=O)Nc1ccccc12] 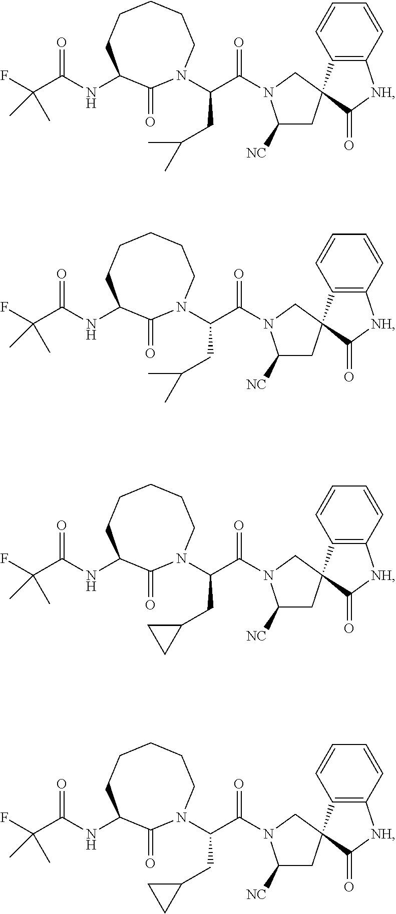 [SMILES: CC(C)C[C@H](C(=O)N1C[C@]2(C[C@H]1C#N)C(=O)Nc1ccccc12)N1CCCCC[C@H](NC(=O)C(C)(C)F)C1=O ; CC(C)C[C@@H](C(=O)N1C[C@]2(C[C@H]1C#N)C(=O)Nc1ccccc12)N1CCCCC[C@H](NC(=O)C(C)(C)F)C1=O ; CC(C)(F)C(=O)N[C@H]1CCCCCN([C@H](CC2CC2)C(=O)N2C[C@]3(C[C@H]2C#N)C(=O)Nc2ccccc23)C1=O ; CC(C)(F)C(=O)N[C@H]1CCCCCN([C@@H](CC2CC2)C(=O)N2C[C@]3(C[C@H]2C#N)C(=O)Nc2ccccc23)C1=O] 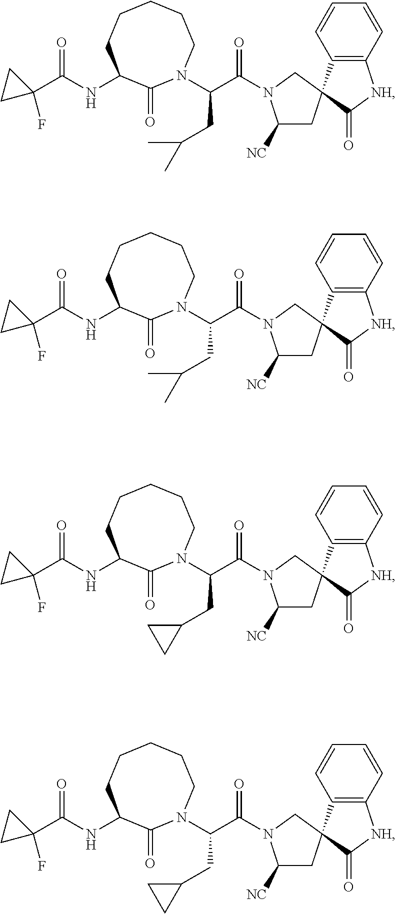 [SMILES: CC(C)C[C@H](C(=O)N1C[C@]2(C[C@H]1C#N)C(=O)Nc1ccccc12)N1CCCCC[C@H](NC(=O)C2(F)CC2)C1=O ; CC(C)C[C@@H](C(=O)N1C[C@]2(C[C@H]1C#N)C(=O)Nc1ccccc12)N1CCCCC[C@H](NC(=O)C2(F)CC2)C1=O ; N#C[C@@H]1C[C@@]2(CN1C(=O)[C@@H](CC1CC1)N1CCCCC[C@H](NC(=O)C3(F)CC3)C1=O)C(=O)Nc1ccccc12 ; N#C[C@@H]1C[C@@]2(CN1C(=O)[C@H](CC1CC1)N1CCCCC[C@H](NC(=O)C3(F)CC3)C1=O)C(=O)Nc1ccccc12] 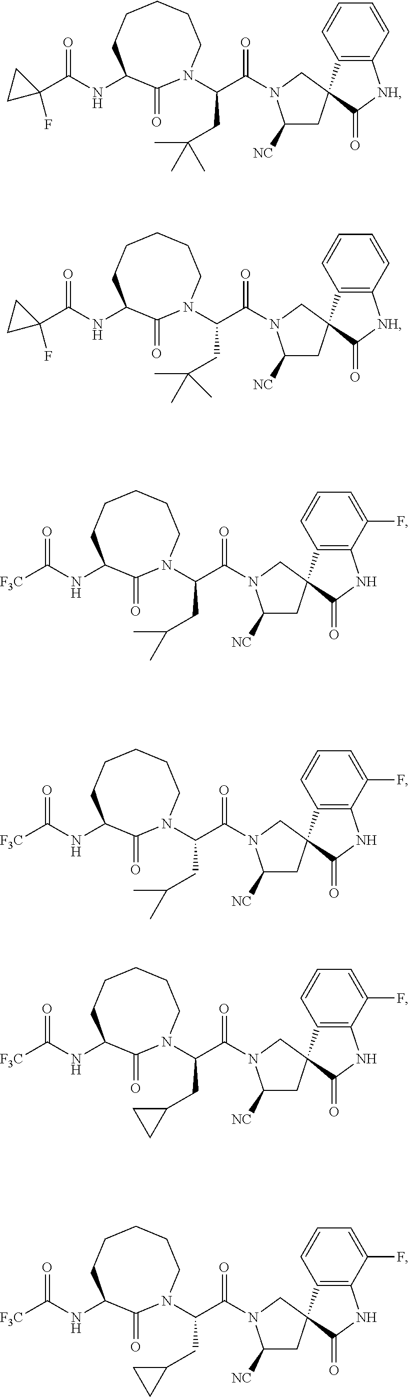 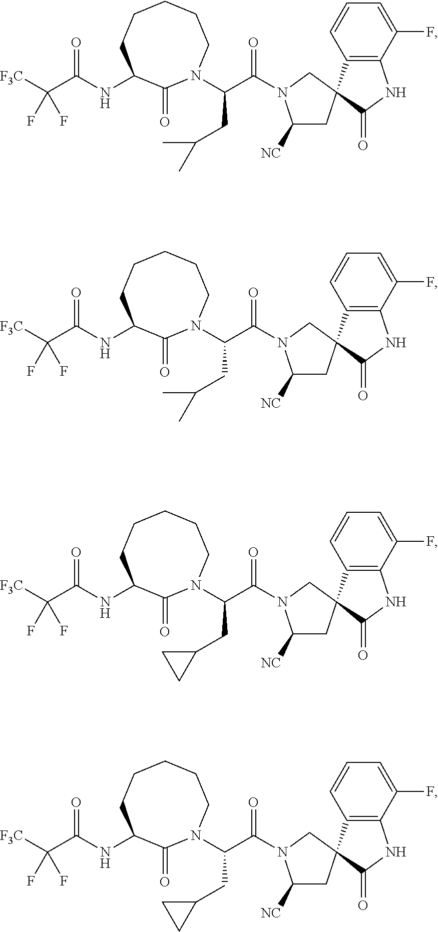 [SMILES: CC(C)C[C@H](C(=O)N1C[C@]2(C[C@H]1C#N)C(=O)Nc1c(F)cccc12)N1CCCCC[C@H](NC(=O)C(F)(F)C(F)(F)F)C1=O ; CC(C)C[C@@H](C(=O)N1C[C@]2(C[C@H]1C#N)C(=O)Nc1c(F)cccc12)N1CCCCC[C@H](NC(=O)C(F)(F)C(F)(F)F)C1=O ; N#C[C@@H]1C[C@@]2(CN1C(=O)[C@@H](CC1CC1)N1CCCCC[C@H](NC(=O)C(F)(F)C(F)(F)F)C1=O)C(=O)Nc1c(F)cccc12 ; N#C[C@@H]1C[C@@]2(CN1C(=O)[C@H](CC1CC1)N1CCCCC[C@H](NC(=O)C(F)(F)C(F)(F)F)C1=O)C(=O)Nc1c(F)cccc12] 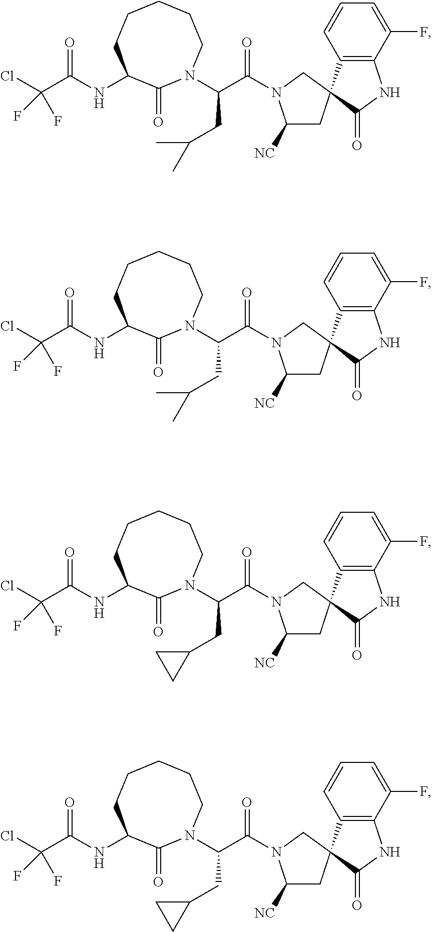 [SMILES: CC(C)C[C@H](C(=O)N1C[C@]2(C[C@H]1C#N)C(=O)Nc1c(F)cccc12)N1CCCCC[C@H](NC(=O)C(F)(F)Cl)C1=O ; CC(C)C[C@@H](C(=O)N1C[C@]2(C[C@H]1C#N)C(=O)Nc1c(F)cccc12)N1CCCCC[C@H](NC(=O)C(F)(F)Cl)C1=O ; N#C[C@@H]1C[C@@]2(CN1C(=O)[C@@H](CC1CC1)N1CCCCC[C@H](NC(=O)C(F)(F)Cl)C1=O)C(=O)Nc1c(F)cccc12 ; N#C[C@@H]1C[C@@]2(CN1C(=O)[C@H](CC1CC1)N1CCCCC[C@H](NC(=O)C(F)(F)Cl)C1=O)C(=O)Nc1c(F)cccc12] 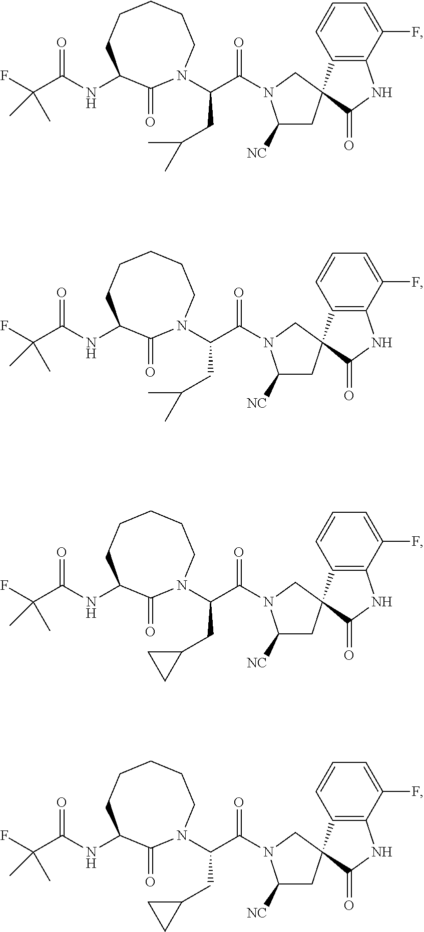 [SMILES: CC(C)C[C@H](C(=O)N1C[C@]2(C[C@H]1C#N)C(=O)Nc1c(F)cccc12)N1CCCCC[C@H](NC(=O)C(C)(C)F)C1=O ; CC(C)C[C@@H](C(=O)N1C[C@]2(C[C@H]1C#N)C(=O)Nc1c(F)cccc12)N1CCCCC[C@H](NC(=O)C(C)(C)F)C1=O ; CC(C)(F)C(=O)N[C@H]1CCCCCN([C@H](CC2CC2)C(=O)N2C[C@]3(C[C@H]2C#N)C(=O)Nc2c(F)cccc23)C1=O ; CC(C)(F)C(=O)N[C@H]1CCCCCN([C@@H](CC2CC2)C(=O)N2C[C@]3(C[C@H]2C#N)C(=O)Nc2c(F)cccc23)C1=O] 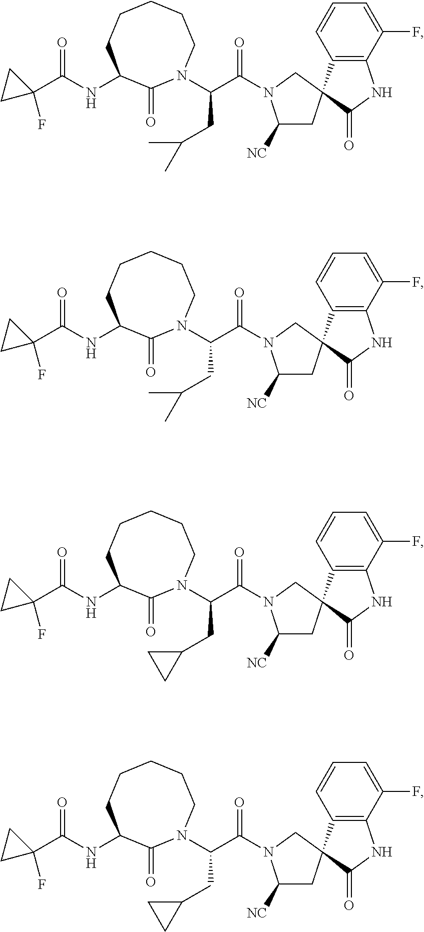 [SMILES: CC(C)C[C@H](C(=O)N1C[C@]2(C[C@H]1C#N)C(=O)Nc1c(F)cccc12)N1CCCCC[C@H](NC(=O)C2(F)CC2)C1=O ; CC(C)C[C@@H](C(=O)N1C[C@]2(C[C@H]1C#N)C(=O)Nc1c(F)cccc12)N1CCCCC[C@H](NC(=O)C2(F)CC2)C1=O ; N#C[C@@H]1C[C@@]2(CN1C(=O)[C@@H](CC1CC1)N1CCCCC[C@H](NC(=O)C3(F)CC3)C1=O)C(=O)Nc1c(F)cccc12 ; N#C[C@@H]1C[C@@]2(CN1C(=O)[C@H](CC1CC1)N1CCCCC[C@H](NC(=O)C3(F)CC3)C1=O)C(=O)Nc1c(F)cccc12] 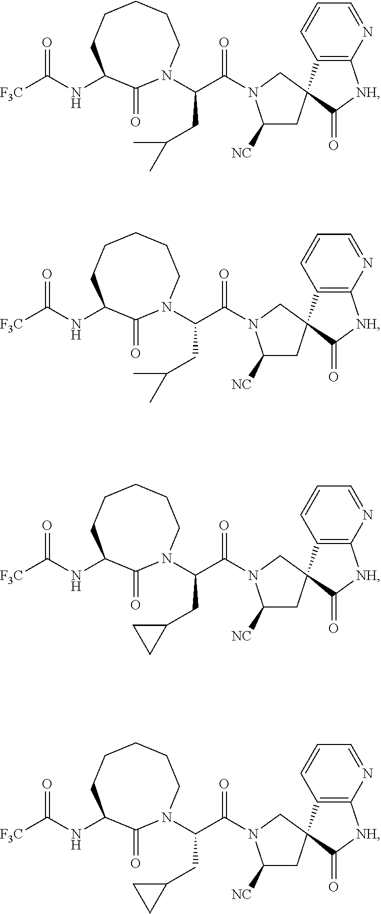 [SMILES: CC(C)C[C@H](C(=O)N1C[C@]2(C[C@H]1C#N)C(=O)Nc1ncccc12)N1CCCCC[C@H](NC(=O)C(F)(F)F)C1=O ; CC(C)C[C@@H](C(=O)N1C[C@]2(C[C@H]1C#N)C(=O)Nc1ncccc12)N1CCCCC[C@H](NC(=O)C(F)(F)F)C1=O ; N#C[C@@H]1C[C@@]2(CN1C(=O)[C@@H](CC1CC1)N1CCCCC[C@H](NC(=O)C(F)(F)F)C1=O)C(=O)Nc1ncccc12 ; N#C[C@@H]1C[C@@]2(CN1C(=O)[C@H](CC1CC1)N1CCCCC[C@H](NC(=O)C(F)(F)F)C1=O)C(=O)Nc1ncccc12] 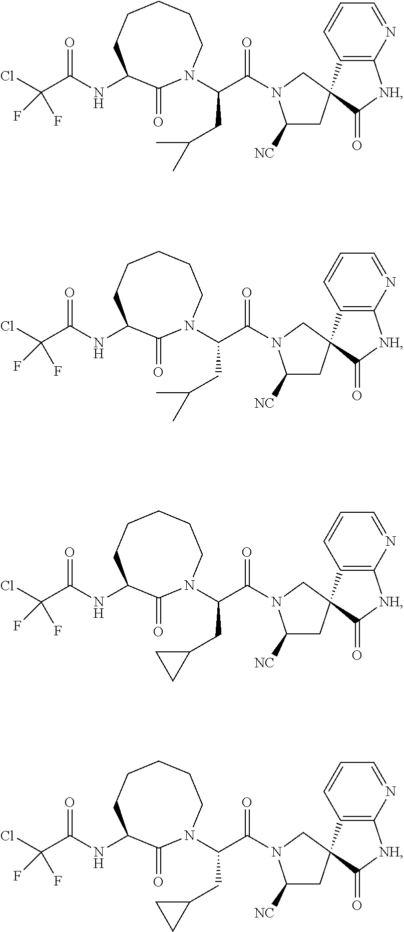 [SMILES: CC(C)C[C@H](C(=O)N1C[C@]2(C[C@H]1C#N)C(=O)Nc1ncccc12)N1CCCCC[C@H](NC(=O)C(F)(F)Cl)C1=O ; CC(C)C[C@@H](C(=O)N1C[C@]2(C[C@H]1C#N)C(=O)Nc1ncccc12)N1CCCCC[C@H](NC(=O)C(F)(F)Cl)C1=O ; N#C[C@@H]1C[C@@]2(CN1C(=O)[C@@H](CC1CC1)N1CCCCC[C@H](NC(=O)C(F)(F)Cl)C1=O)C(=O)Nc1ncccc12 ; N#C[C@@H]1C[C@@]2(CN1C(=O)[C@H](CC1CC1)N1CCCCC[C@H](NC(=O)C(F)(F)Cl)C1=O)C(=O)Nc1ncccc12] 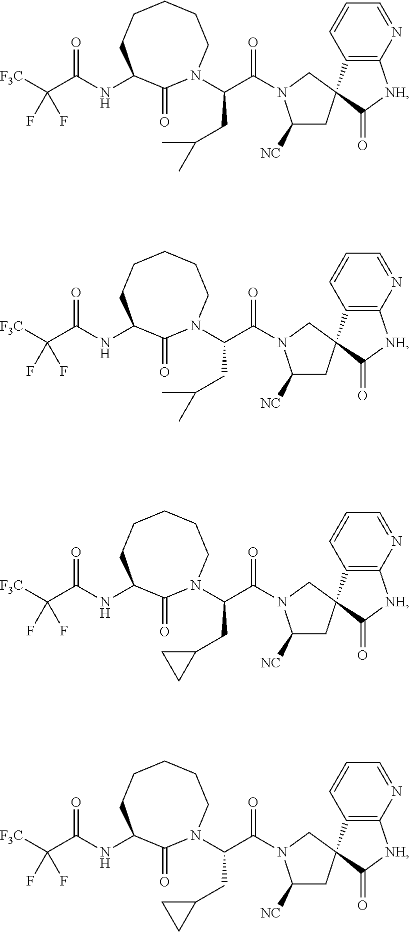 [SMILES: CC(C)C[C@H](C(=O)N1C[C@]2(C[C@H]1C#N)C(=O)Nc1ncccc12)N1CCCCC[C@H](NC(=O)C(F)(F)C(F)(F)F)C1=O ; CC(C)C[C@@H](C(=O)N1C[C@]2(C[C@H]1C#N)C(=O)Nc1ncccc12)N1CCCCC[C@H](NC(=O)C(F)(F)C(F)(F)F)C1=O ; N#C[C@@H]1C[C@@]2(CN1C(=O)[C@@H](CC1CC1)N1CCCCC[C@H](NC(=O)C(F)(F)C(F)(F)F)C1=O)C(=O)Nc1ncccc12 ; N#C[C@@H]1C[C@@]2(CN1C(=O)[C@H](CC1CC1)N1CCCCC[C@H](NC(=O)C(F)(F)C(F)(F)F)C1=O)C(=O)Nc1ncccc12] 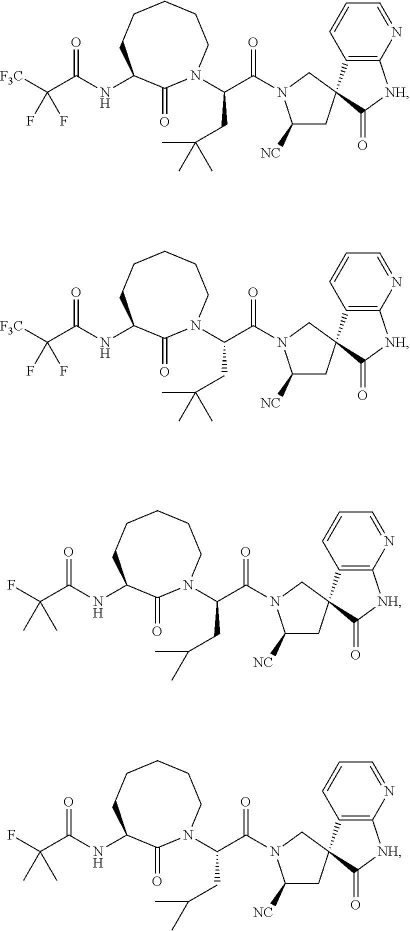 [SMILES: CC(C)(C)C[C@H](C(=O)N1C[C@]2(C[C@H]1C#N)C(=O)Nc1ncccc12)N1CCCCC[C@H](NC(=O)C(F)(F)C(F)(F)F)C1=O ; CC(C)(C)C[C@@H](C(=O)N1C[C@]2(C[C@H]1C#N)C(=O)Nc1ncccc12)N1CCCCC[C@H](NC(=O)C(F)(F)C(F)(F)F)C1=O ; CC(C)C[C@H](C(=O)N1C[C@]2(C[C@H]1C#N)C(=O)Nc1ncccc12)N1CCCCC[C@H](NC(=O)C(C)(C)F)C1=O ; CC(C)C[C@@H](C(=O)N1C[C@]2(C[C@H]1C#N)C(=O)Nc1ncccc12)N1CCCCC[C@H](NC(=O)C(C)(C)F)C1=O] 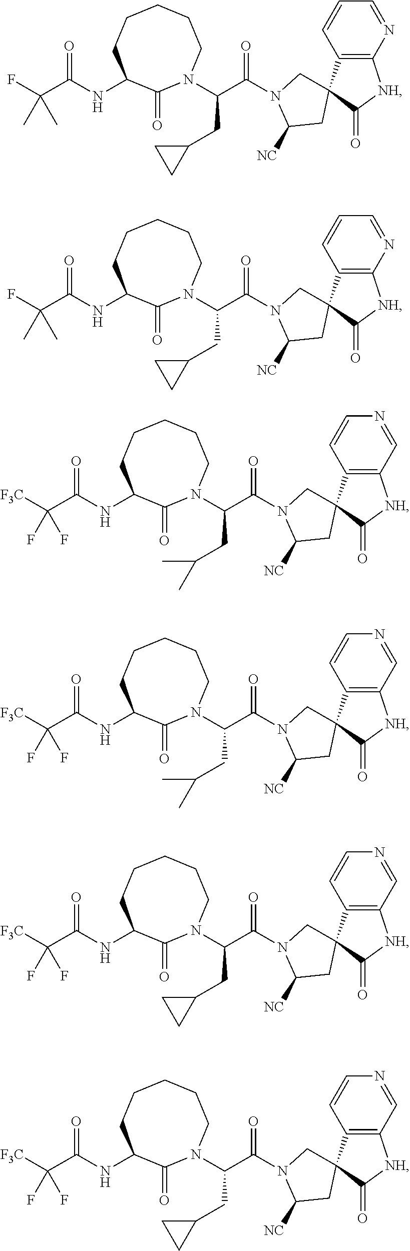 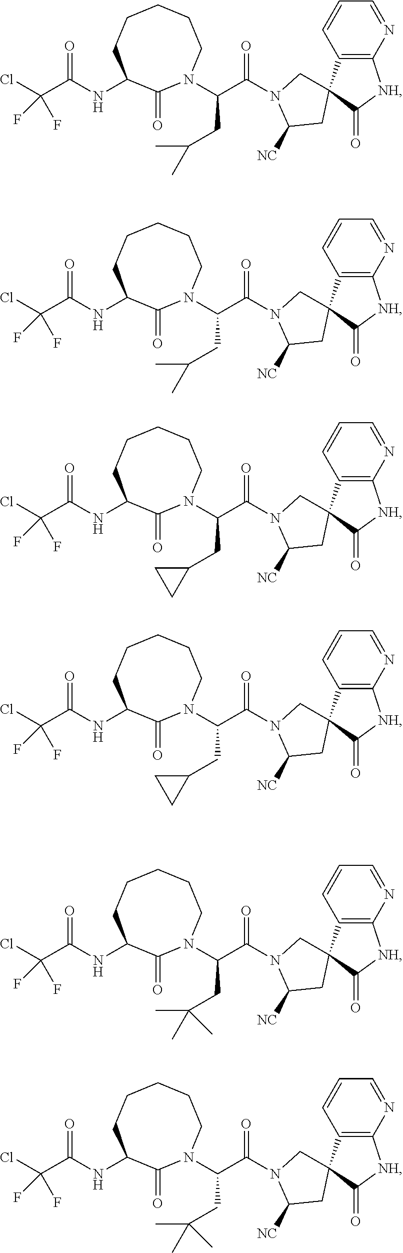 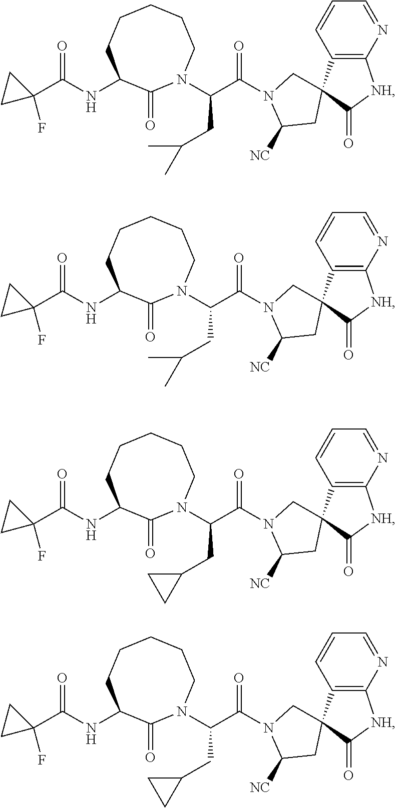 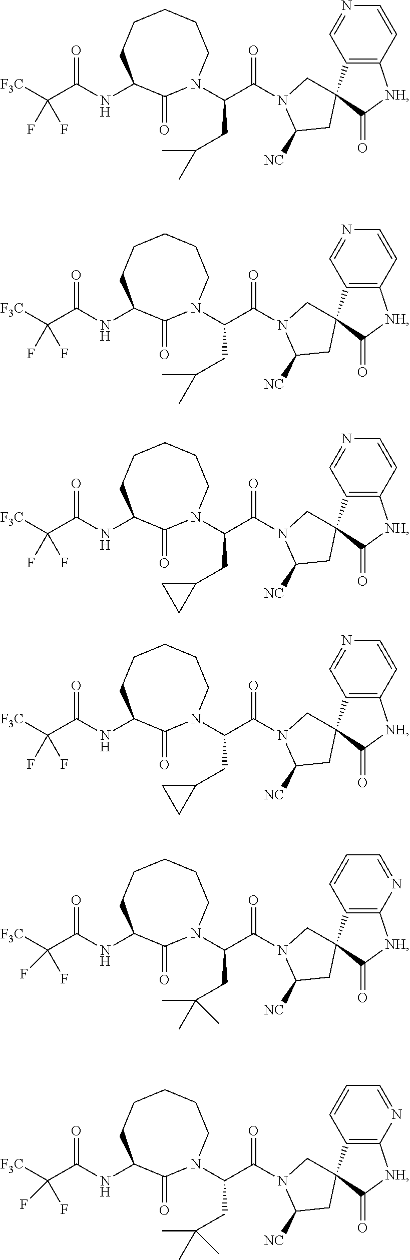 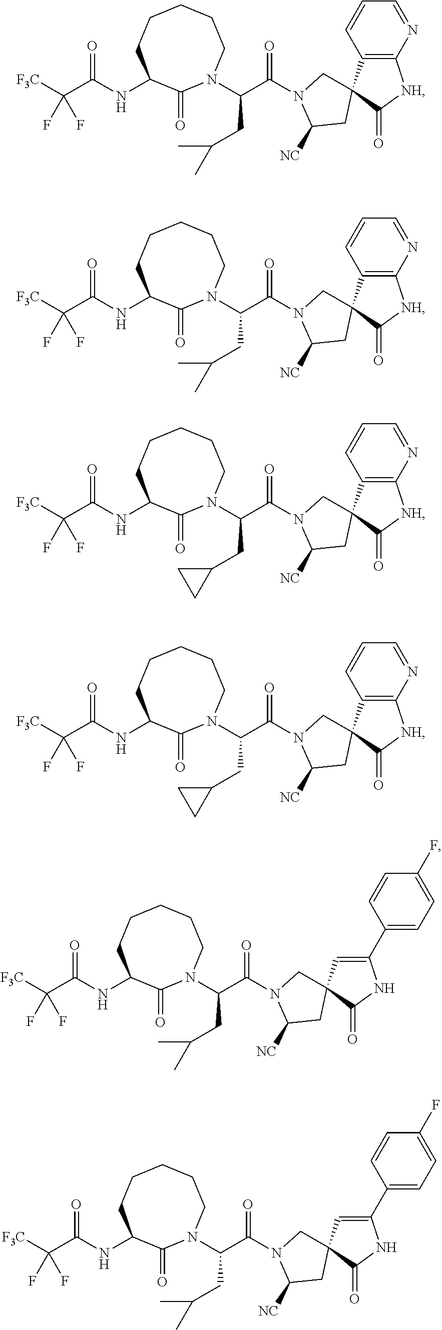 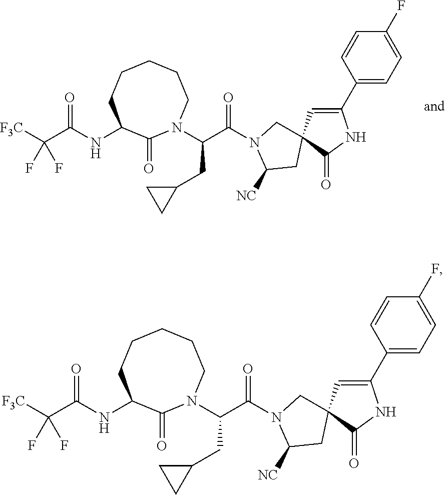 [SMILES: N#C[C@@H]1C[C@@]2(C=C(c3ccc(F)cc3)NC2=O)CN1C(=O)[C@@H](CC1CC1)N1CCCCC[C@H](NC(=O)C(F)(F)C(F)(F)F)C1=O ; N#C[C@@H]1C[C@@]2(C=C(c3ccc(F)cc3)NC2=O)CN1C(=O)[C@H](CC1CC1)N1CCCCC[C@H](NC(=O)C(F)(F)C(F)(F)F)C1=O]
or a pharmaceutically acceptable salt of any of the foregoing.

86. A pharmaceutical composition comprising an effective amount of a compound of claim 1, or a pharmaceutically acceptable salt thereof, and an excipient.

87.-102. (canceled)

103. A method for treating a coronavirus infection in a subject comprising administering to the subject in need thereof an effective amount of a compound of claim 1, or a pharmaceutically acceptable salt thereof.

104. The method of claim 103, further comprising administering an additional agent selected from the group consisting of an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine.

105.-109. (canceled)

110. A method for treating a norovirus or picornavirus infection in a subject comprising administering to the subject in need thereof an effective amount of a compound of claim 1, or a pharmaceutically acceptable salt thereof.

111.-119. (canceled)

120. A method for inhibiting a coronavirus protease comprising contacting a cell infected with a coronavirus with an effective amount of a compound of claim 1, or a pharmaceutically acceptable salt thereof, wherein the compound claim 1, or a pharmaceutically acceptable salt thereof, selectively inhibits the coronavirus protease compared to a host protease.

121. (canceled)

122. (canceled)

123. (canceled)
